(19) United States
(12) Patent Application Publication
(10) Pub. No.: US 2012/0164695 A1
(43) Pub. Date: Jun. 28, 2012

(54) COMBINATORIAL VARIANTS OF GLUCOAMYLASE WITH IMPROVED SPECIFIC ACTIVITY AND/OR THERMOSTABILITY

(75) Inventors: Wolfgang Aehle, Zwingenberg (DE); Richard R. Bott, City Burlingame, CA (US); Martijn Silvan Scheffers, City Leiden (NL); Casper Vroeman, Oegstgeest (NL)

(73) Assignee: DANISCO US INC., Palo Alto, CA (US)

(21) Appl. No.: 13/390,381

(22) PCT Filed: Aug. 18, 2010
(86) PCT No.: PCT/US10/45865

§ 371 Date: Mar. 15, 2012

Related U.S. Application Data
(60) Provisional application No. 61/235,140, filed on Aug. 19, 2009.

(51) Int. Cl. C12N 9/34; C12N 15/63; C12P 19/14; C12N 5/10; C12N 1/15; C12N 1/21; C12N 1/19; C12N 15/56; C12P 7/02

(52) U.S. Cl. 435/99; 435/205; 536/23.2; 435/320.1; 435/155; 435/254.11; 435/254.6; 435/254.5; 435/254.3; 435/254.7; 435/254.4; 435/254.8; 435/254.9; 435/252.35; 435/252.31; 435/252.33; 435/252.34; 435/254.21; 435/254.2; 435/254.23; 435/254.22; 435/418; 435/412; 435/415

(57) ABSTRACT

Presently provided are variant glucoamylases displaying altered properties, such as improved thermostability and/or specific activity. Also disclosed are DNA sequences coding for the variants, vectors and host cells incorporating the DNA sequence, enzyme compositions, and methods of using the variants in various applications.

### PRIORITY

The present application claim priority to U.S. Provisional Application Ser. No. 61/235,140 filed on Aug. 19, 2009, which is hereby incorporated by reference in its entirety.

### SEQUENCE LISTING

Also attached is a sequence listing comprising SEQ ID NOs: 1-1097, which are herein incorporated by reference in their entirety.

### FIELD OF THE INVENTION

Disclosed are combinatorial variants of a parent glucoamylase that have altered properties and are suitable for starch hydrolyzing compositions and cleaning compositions. Also disclosed are DNA constructs encoding the variants and methods of producing the glucoamylase variants in host cells.

### BACKGROUND

Glucoamylase enzymes (glucan 1,4-α-glucohydrolases, EC 3.2.1.3) are starch hydrolyzing exo-acting carbohydrases, which catalyze the removal of successive glucose units from the non-reducing ends of starch or related oligo and polysaccharide molecules. Glucoamylases can hydrolyze both the linear and branched glucosidic linkages of starch (e.g., amylose and amylopectin).

Glucoamylases are produced by numerous strains of bacteria, fungi, yeast and plants. Particularly interesting, and commercially important, glucoamylases are fungal enzymes that are extracellularly produced, for example from strains of Aspergillus (Svensson et al., Carlsberg Res. Commun. 48: 529-544 (1983); Boel et al., EMBO J. 3: 1097-1102 (1984); Hayashida et al., Agric. Biol. Chem. 53: 923-929 (1989); U.S. Pat. No. 5,024,941; U.S. Pat. No. 4,794,175 and WO 88/09795); Talaromyces (U.S. Pat. No. 4,247,637; U.S. Pat. No. 6,255,084; and U.S. Pat. No. 6,620,924); Rhizopus (Ashikari et al., Agric. Biol. Chem. 50: 957-964 (1986); Ashikari et al., App. Microbio. Biotech. 32: 129-133 (1989) and U.S. Pat. No. 4,863,864); Humicola (WO 05/052148 and U.S. Pat. No. 4,618,579); and Mucor (Houghton-Larsen et al., Appl. Microbiol. Biotechnol. 62: 210-217 (2003)). Many of the genes that code for these enzymes have been cloned and expressed in yeast, fungal and/or bacterial cells.

Commercially, glucoamylases are very important enzymes and have been used in a wide variety of applications that require the hydrolysis of starch (e.g., for producing glucose and other monosaccharides from starch). Glucoamylases are used to produce high fructose corn sweeteners, which comprise over 50% of the sweetener market in the United States. In general, glucoamylases may be, and commonly are, used with alpha-amylases in starch hydrolyzing processes to hydrolyze starch to dextrins and then glucose. The glucose may then be converted to fructose by other enzymes (e.g., glucose isomerases); crystallized; or used in fermentations to produce numerous end products (e.g., ethanol, citric acid, lactic acid, succinate, ascorbic acid intermediates, glutamic acid, glycerol and 1,3-propanediol). Ethanol produced by using glucoamylases in the fermentation of starch and/or cellulose containing material may be used as a source of fuel or for alcoholic consumption.

Although glucoamylases have been used successfully in commercial applications for many years, a need still exists for new glucoamylases with altered properties, such as improved specific activity and increased thermostability.

### SUMMARY

The glucoamylase variants as contemplated herein contain amino acid substitutions within the catalytic domains and/or the starch binding domain. The variants display altered properties, such as improved thermostability and/or increased specific activity.

In one aspect, the glucoamylase variant comprises one or more substitutions in the following positions in the amino acid sequence set forth in SEQ ID NO: 2: 10, 14, 15, 23, 42, 43, 44, 59, 60, 61, 65, 67, 68, 72, 73, 97, 98, 99, 102, 110, 113, 114, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 294, 300, 301, 303, 311, 338, 342, 344, 346, 349, 359, 361, 364, 375, 379, 382, 390, 391, 393, 394, 410, 417, 418, 430, 431, 433, 436, 442, 444, 448, 451, 493, 494, 495, 502, 503, 508, 511, 518, 519, 520, 527, 531, 535, 536, 537, 539, 563, or 577, or in an equivalent position in a parent glucoamylase. The one or more amino acid substitutions can be: T10, L14, N15, P23, T42, I43, D44, P45, D46, F59, K60, N61, T67, E68, A72, G73, S97, L98, A99, S102, K108, E110, L113, K114, R122, Q124, R125, I133, K140, N144, N145, Y147, S152, N153, N164, F175, N182, A204, T205, S214, V216, Q219, W228, V229, S230, S231, D236, I239, N240, T241, N242, G244, N263, L264, G265, A268, G269, D276, V284, S291, G294, P300, A301, A303, Y310, A311, D313, Y316, V338, T342, S344, T346, A349, V359, G361, A364, T375, N379, S382, S390, E391, A393, K394, R408, S410, S415, L417, H418, T430, A431, R433, 1436, A442, N443, S444, T448, S451, T493, P494, T495, H502, E503, Q508, Q511, N518, A519, A520, T527, V531, A535, V536, N537, A539, N563, and N577. In a further aspect, the one or more amino acid substitutions can be: T10S, T42V, I43Q/R, D44R/C, N61I, T67M, E68C/M, A72Y, G73F/W, S97N, S102A/M/R, K114M/Q, I133T/V, N145I, N153A/D/E/M/S/V, T205Q, Q219S, W228A/F/H/M/V, V229I/L, S230C/F/G/L/N/Q/R, S231L/V, D236R, I239V/Y, N263P, L264D/K, A268C/D/G/K, S291A/F/H/M/T, G294C, A301P/R, V338I/N/Q, T342V, S344M/P/Q/R/V, G361D/E/F/I/L/M/P/S/W/Y, A364D/E/F/G/K/L/M/R/S/T/V/W, T375N, K394S, L417K/R/V, T430A/K, A431I/L/Q, R433c/E/G/L/N/S/V/Y, I436H, T451K, T495K/M/S, E503A/C/V, Q508R, Q511H, A519I/K/R/Y, A520C/L/P, V531L, A535K/N/P/R, V536M, A539E/R/S, N563C/E/I/K/K/Q/T/V, or N577K/P/R.

In another aspect, the glucoamylase variant comprises two or more amino acid substitutions corresponding to position: 43, 44, 61, 73, 294, 417, 430, 431, 503, 511, 535, 539, or 563 of SEQ ID NO: 2, or equivalent positions in a parent glucoamylase. The two or more amino acid substitutions can be: I43Q/R, D44R/C, N61I, G73F, G294C, L417R/V, T430A/M, A431L/Q, E503A/V, Q511H, A535R, A539R, and/or N563I/K. In yet another aspect, the glucoamylase variant may further comprises one or more amino acid substitutions corresponding to position: 10, 14, 15, 23, 42, 59, 60, 65, 67, 68, 72, 97, 98, 99, 102, 110, 113, 114, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 418, 433, 436, 442, 444, 448, 451, 493, 494, 495, 502, 508, 518, 519, 520, 527, 531, 536, 537, or 577 of SEQ ID NO: 2, or equivalent position in a parent glucoamylase. The one or more additional amino acid substitutions can be: T10S, T42V, T67M, E68C/M, A72Y, S97N, S102A/M/R, K114M/Q, I133T/V, N145I, N153A/D/E/M/S/V, T205Q, Q219S, W228A/F/H/M/V, V229I/L, S230C/F/G/L/N/Q/R, S231L/V, D236R, I239V/Y, N263P, L264D/K, A268C/D/G/K, S291A/F/H/M/T, A301P/R, V338I/N/Q, T342V, S344M/P/Q/R/V, G361D/E/F/I/L/M/P/S/W/Y, A364D/E/F/G/K/L/M/R/S/T/V/W, T375N, K394S, R433c/E/G/L/N/S/V/Y, I436H, T451K, T495K/M/S, Q508R, A519I/K/R/Y, A520C/L/P, V531L, V536M, or N577K/P/R.

In further aspects, the glucoamylase variant comprises amino acids substitutions corresponding to positions: (a) 61, 417, 431, and 539, (b) 43, 417, 431, 535, and 539; or (c) 73, 503, and 563 of SEQ ID NO: 2 or equivalent positions in a parent glucoamylase. The amino acids substitutions can be: (a) N61I, L417G/R/V, A431L/Q, and A539R; (2) I43Q/R, L417G/R/V, A431L/Q, A535R, and A539R; or (3) G73F, E503V, and N563K.

The glucoamylase variant may comprise one of the following sets of substitutions, at the relevant positions of SEQ ID NO: 2, or at equivalent positions in a parent glucoamylase:

### N61I/L417V/A431L/A539R;

### I43Q/N61I/L417V/A431L/A539R;

### N61I/L417V/A431L/A535R/A539R

### I43Q/L417V/A431L/A535R/A539R;

### I43Q/N61I/L417V/A431L/A535R/A539R;

### I43Q/N61I/L417V/T430A/A431L/A535R/A539R;

### I43Q/L417V/T430A/A431L/Q511H/A535R/A539R/N5631;

### N61I/L417V/T430A/A431L/Q511H/A535R/A539R/N5631;

### I43Q/N61I/L417V/T430A/A431L/Q511H/A535R/A539R/N563I;

### I43R/N61I/L417V/A431L/A539R;

### I43R/N61I/L417V/T430A/A431L/A535R/A539R;

### G73F/L417R/E503V/A539R/N563K;

### I43R/G73F/L417R/E503V/A539R/N563K; and

### I43R/G73F/E503V/Q511H/N563K.

The glucoamylase variant may comprise one or more additional amino acid substitutions corresponding to positions: 43, 44, 61, 73, 294, 430, 503, 511, 535, or 563 of SEQ ID NO: 2, or an equivalent position in the parent glucoamylase. The one or more additional amino acid substitutions can be: I43Q/R, D44C/R, N61I, G73F, G294C, T430A/M, E503A/V, Q511H, A535R, or N563I/K. The glucoamylase variant may comprise one or more additional amino acid substitutions corresponding to positions: 10, 14, 15, 23, 42, 59, 60, 65, 67, 68, 72, 97, 98, 99, 102, 110, 113, 114, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 418, 433, 436, 442, 444, 448, 451, 493, 494, 495, 502, 508, 518, 519, 520, 527, 531, 536, 537, or 577 of SEQ ID NO: 2, or an equivalent position in the parent glucoamylase. The one or more additional amino acid substitutions can be: T105, T42V, T67M, E68C/M, A72Y, S97N, 5102A/M/R, K114M/Q, I133T/V, N145I, N153A/D/E/M/S/V, T205Q, Q2195, W228A/F/H/M/V, V229I/L, S230C/F/G/L/N/Q/R, S231L/V, D236R, I239V/Y, N263P, L264D/K, A268C/D/G/K, S291A/F/H/M/T, A301P/R, V338I/N/Q, T342V, S344M/P/Q/R/V, G361D/E/F/I/L/M/P/S/W/Y, A364D/E/F/G/K/L/M/R/S/T/V/W, T375N, K394S, R433c/E/G/L/N/S/V/Y, I436H, T451K, T495K/M/S, Q508R, A519I/K/R/Y, A520C/L/P, V531L, V536M, or N577K/P/R.

The glucoamylase variant has at last 80%, 85%, 90%, 95%, 99.5% sequence identity with SEQ ID NO: 1 or 2, or the parent glucoamylase. The parent glucoamylase or the glucoamylase variant may comprise a catalytic domain that has at least 80%, 85%, 90%, 95%, or 99.5% sequence identity with SEQ ID NO: 1, 2, 3, 5, 6, 7, 8, or 9. The parent glucoamylase or the glucoamylase variant may comprise a starch binding domain that has at least 95%, 96%, 97%, 98%, 99%, or 99.5% sequence identity with SEQ ID NO: 1, 2, 11, 385, 386, 387, 388, 389, or 390. The parent glucoamylase may comprise SEQ ID: 1 or 2. Optionally, the parent glucoamylase may consist of SEQ ID NO: 1 or 2. The parent glucoamylase can be the enzyme obtained from any of: a Trichoderma spp., an Aspergillus spp., a Humicola spp., a Penicillium spp., a Talaromycese spp., or a Schizosaccharmyces spp. In some aspects, the parent glucoamylase can be from a Trichoderma spp. or an Aspergillus spp.

The present invention further provides glucoamylase variant comprising one of the following sets of substitutions, at positions of SEQ ID NO: 2 or equivalent positions in a parent glucoamylase:

L417V/A431L/A539R;

I43Q/L417V/A431L/A539R;

L417V/A431L/A535R/A539R

I43R/L417V/A431L/A539R;

L417R/A431L/A539R; or

L417G/A431L/A539R;

wherein the glucoamylase variant does not have any further substitutions relative to the parent glucoamylase, and wherein the parent glucoamylase has a catalytic domain that has at least 80% sequence identity with SEQ ID NO: 1, 2, 3, 5, 6, 7, 8, or 9.

The parent glucoamylase may comprise a starch binding domain that has at least 95% sequence identity with SEQ ID NO: 1, 2, 11, 385, 386, 387, 388, 389, or 390. The parent glucoamylase may have at least 80% sequence identity with SEQ ID NO: 1 or 2; for example it may comprise SEQ ID NO: 1 or 2. Optionally the parent glucoamylase may consist of SEQ ID NO: 1 or 2.

In one aspect, the variant glucoamylase exhibits altered thermostability as compared to the parent glucoamylase. The altered thermostability can be increased thermostability. Alternatively, or in addition, the variant exhibits altered specific activity compared to the parent glucoamylase. The altered specific activity can be increased specific activity.

The present disclosure further relates to a polynucleotide encoding the variant described. One aspect is a vector comprising the polynucleotide. Another aspect is a host cell containing the vector. A further aspect is a method of producing a variant glucoamylase by culturing the host cell containing the polynucleotide under conditions suitable for the expression and production of the glucoamylase variant and producing the variant. The method may also include the step of recovering the glucoamylase variant from the culture.

A further aspect of the disclosure is an enzyme composition including the glucoamylase variant. In one aspect, the enzyme composition is used in a starch conversion process, such as an alcohol fermentation process or a high glucose syrup production process.

### BRIEF DESCRIPTION OF THE DRAWINGS

The accompanying drawings are incorporated in and constitute a part of this specification, illustrate embodiments. In the drawings:

FIG. 1A depicts a Trichoderma reesei glucoamylase (TrGA) having 632 amino acids (SEQ ID NO: 1). The signal peptide is underlined, the catalytic region (SEQ ID NO: 3) starting with amino acid residues SVDDFI (SEQ ID NO: 12) and having 453 amino acid residues is in bold; the linker region is in italics and the starch binding domain (SBD) is both italics and underlined. The mature protein of TrGA (SEQ ID NO: 2) includes the catalytic domain (SEQ ID NO: 3), linker region (SEQ ID NO: 10), and starch binding domain (SEQ ID NO: 11). With respect to the SBD numbering of the TrGA glucoamylase molecule, reference is made in the present disclosure to either a) positions 491 to 599 in SEQ ID NO:2 of the mature TrGA, and/or b) positions 1 to 109 in SEQ ID NO:11, which represents the isolated SBD sequence of the mature TrGA. With respect to the catalytic domain numbering of the TrGA molecule, reference is made to SEQ ID NO: 2 and/or SEQ ID NO: 3. FIG. 1B depicts the cDNA (SEQ ID NO:4) that codes for the TrGA. FIG. 1C depicts the precursor and mature protein TrGA domains.

FIG. 2 depicts the destination plasmid pDONR-TrGA which includes the cDNA (SEQ ID NO: 4) of the TrGA.

FIG. 3 depicts the plasmid pTTT-Dest.

FIG. 4 depicts the final expression vector pTTT-TrGA.

FIGS. 5A and 5B depict an alignment comparison of the catalytic domains of parent glucoamylases from Aspergillus awamori (AaGA) (SEQ ID NO: 5); Aspergillus niger (AnGA) (SEQ ID NO: 6); Aspergillus oryzae (AoGA) (SEQ ID NO: 7); Trichoderma reesei (TrGA) (SEQ ID NO: 3); Humicola grisea (HgGA) (SEQ ID NO: 8); and Hypocrea vinosa (HvGA) (SEQ ID NO: 9). Identical amino acids are indicated by an asterisk (*). FIG. 5C depicts a Talaromyces glucoamylase (TeGA) mature protein sequence (SEQ ID NO: 384). FIGS. 5D and 5E depict an alignment comparing the Starch Binding Domain (SBD) of parent glucoamylases from Trichoderma reesei (SEQ ID NO: 11); Humicola grisea (HgGA) (SEQ ID NO: 385); Thermomyces lanuginosus (ThGA) (SEQ ID NO: 386); Talaromyces emersonii (TeGA) (SEQ ID NO: 387); Aspergillus niger (AnGA) (SEQ ID NO: 388); Aspergillus awamori (AaGA) (SEQ ID NO: 389); and Thielavia terrestris (TtGA) (SEQ ID NO: 390).

FIG. 6 depicts a comparison of the three dimensional structure of Trichoderma reesei glucoamylase (black) (SEQ ID NO: 2) and Aspergillus awamori glucoamylase (grey) (SEQ ID NO: 5) viewed from the side. The side is measured in reference to the active site and the active site entrance is at the “top” of the molecule.

FIG. 7 depicts a comparison of the three dimensional structures of Trichoderma reesei glucoamylase (black) (SEQ ID NO: 2) and Aspergillus awamori glucoamylase (grey) (SEQ ID NO: 5) viewed from the top.

FIG. 8 depicts an alignment of the three dimensional structures of TrGA (SEQ ID NO: 2) and AnGA (SEQ ID NO: 6) viewed from the side showing binding sites 1 and 2.

FIG. 9 depicts a model of the binding of acarbose to the TrGA crystal structure.

### DETAILED DESCRIPTION

Glucoamylases are commercially important enzymes in a wide variety of applications that require the hydrolysis of starch. Glucoamylase variants described herein contain amino acid substitutions within either the catalytic domain or the starch binding domain. The variants may display altered properties such as improved thermostability and/or specific activity. The variants with improved thermostability and/or specific activity may significantly improve the efficiency of glucose and fuel ethanol production from corn starch, for example.

1. Definitions and Abbreviations

1.1. Definitions

Unless defined otherwise, all technical and scientific terms used herein have the same meaning as commonly understood by one of ordinary skill in the art to which this disclosure belongs. Singleton et al., DICTIONARY OF MICROBIOLOGY AND MOLECULAR BIOLOGY, 2nd ed., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Certain terms are defined below for the sake of clarity and ease of reference.

As used herein, the term “glucoamylase (EC 3.2.1.3)” refers to an enzyme that catalyzes the release of D-glucose from the non-reducing ends of starch and related oligo- and polysaccharides.

The term “parent” or “parent sequence” refers to a sequence that is native or naturally occurring in a host cell. Parent glucoamylases include, but are not limited to, the glucoamylase sequences set forth in SEQ ID NOs: 1, 2, 3, 5, 6, 7, 8, and 9, and glucoamylases with 80% amino acid sequence identity to SEQ ID NO: 2.

As used herein, an “equivalent position” means a position that is common to two parent sequences that is based on an alignment of the amino acid sequence of the parent glucoamylase in question as well as alignment of the three-dimensional structure of the parent glucoamylase in question with the TrGA reference glucoamylase amino acid sequence (SEQ ID NO: 2) and three-dimensional structure. Thus either sequence alignment or structural alignment may be used to determine equivalence.

The term “TrGA” refers to a parent Trichoderma reesei glucoamylase sequence having the mature protein sequence illustrated in SEQ ID NO: 2 that includes the catalytic domain having the sequence illustrated in SEQ ID NO: 3. The isolation, cloning and expression of the TrGA are described in WO 2006/060062 and U.S. Pat. No. 7,413,887, both of which are incorporated herein by reference. In some embodiments, the parent sequence refers to a glucoamylase sequence that is the starting point for protein engineering. The numbering of the glucoamylase amino acids herein is based on the sequence alignment of a glucoamylase with TrGA (SEQ ID NO: 2 and/or 3).

The phrase “mature form of a protein or polypeptide” refers to the final functional form of the protein or polypeptide. A mature form of a glucoamylase may lack a signal peptide, for example. To exemplify, a mature form of the TrGA includes the catalytic domain, linker region and starch binding domain having the amino acid sequence of SEQ ID NO: 2.

As used herein, the terms “glucoamylase variant” and “variant” are used in reference to glucoamylases that have some degree of amino acid sequence identity to a parent glucoamylase sequence. A variant is similar to a parent sequence, but has at least one substitution, deletion or insertion in their amino acid sequence that makes them different in sequence from a parent glucoamylase. In some cases, variants have been manipulated and/or engineered to include at least one substitution, deletion, or insertion in their amino acid sequence that makes them different in sequence from a parent. Additionally, a glucoamylase variant may retain the functional characteristics of the parent glucoamylase, e.g., maintaining a glucoamylase activity that is at least about 50%, about 60%, about 70%, about 80%, or about 90% of that of the parent glucoamylase.

“Variants” may have at least about 45%, about 50%, about 55%, about 60%, about 65%, about 70%, about 75%, about 80%, about 85%, about 88%, about 90%, about 91%, about 92%, about 93%, about 94%, about 95%, about 96%, about 97%, about 98%, about 99%, or about 99.5% sequence identity to a parent polypeptide sequence when optimally aligned for comparison. In some embodiments, the glucoamylase variant may have at least about 45%, about 50%, about 55%, about 60%, about 65%, about 70%, about 75%, about 80%, about 85%, about 88%, about 90%, about 91%, about 92%, about 93%, about 94%, about 95%, about 96%, about 97%, about 98%, about 99%, or about 99.5% sequence identity to the catalytic domain of a parent glucoamylase. In some embodiments, the glucoamylase variant may have at least at least about 45%, about 50%, about 55%, about 60%, about 65%, about 70%, about 75%, about 80%, about 85%, about 88%, about 90%, about 91%, about 92%, about 93%, about 94%, about 95%, about 96%, about 97%, about 98%, about 99%, or about 99.5% sequence identity to the starch binding domain of a parent glucoamylase. The sequence identity can be measured over the entire length of the parent or the variant sequence.

Sequence identity is determined using standard techniques known in the art (see e.g., Smith and Waterman, Adv. Appl. Math. 2: 482 (1981); Needleman and Wunsch, J. Mol. Biol. 48: 443 (1970); Pearson and Lipman, Proc. Natl. Acad. Sci. USA 85: 2444 (1988); programs such as GAP, BESTHT, FASTA, and TFASTA in the Wisconsin Genetics Software Package (Genetics Computer Group, Madison, Wis.); and Devereux et al., Nucleic Acid Res., 12: 387-395 (1984)).

The “percent (%) nucleic acid sequence identity” or “percent (%) amino acid sequence identity” is defined as the percentage of nucleotide residues or amino acid residues in a candidate sequence that are identical with the nucleotide residues or amino acid residues of the starting sequence (e.g., PS4). The sequence identity can be measured over the entire length of the starting sequence.

“Sequence identity” is determined herein by the method of sequence alignment. For the purpose of the present disclosure, the alignment method is BLAST described by Altschul et al., (Altschul et al., J. Mol. Biol. 215: 403-410 (1990); and Karlin et al, Proc. Natl. Acad. Sci. USA 90: 5873-5787 (1993)). A particularly useful BLAST program is the WU-BLAST-2 program (see Altschul et al, Meth. Enzymol. 266: 460-480 (1996)). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The term “optimal alignment” refers to the alignment giving the highest percent identity score.

As used herein the term “catalytic domain” refers to a structural region of a polypeptide, which contains the active site for substrate hydrolysis.

The term “linker” refers to a short amino acid sequence generally having between 3 and 40 amino acids residues that covalently bind an amino acid sequence comprising a starch binding domain with an amino acid sequence comprising a catalytic domain.

The term “starch binding domain” refers to an amino acid sequence that binds preferentially to a starch substrate.

As used herein, the terms “mutant sequence” and “mutant gene” are used interchangeably and refer to a polynucleotide sequence that has an alteration in at least one codon occurring in a host cell's parent sequence. The expression product of the mutant sequence is a variant protein with an altered amino acid sequence relative to the parent. The expression product may have an altered functional capacity (e.g., enhanced enzymatic activity).

The term “property” or grammatical equivalents thereof in the context of a polypeptide, as used herein, refers to any characteristic or attribute of a polypeptide that can be selected or detected. These properties include, but are not limited to oxidative stability, substrate specificity, catalytic activity, thermal stability, pH activity profile, resistance to proteolytic degradation, K_{M}, K_{CAT}, K_{CAT}/K_{M} ratio, protein folding, ability to bind a substrate and ability to be secreted.

The term “property” of grammatical equivalent thereof in the context of a nucleic acid, as used herein, refers to any characteristic or attribute of a nucleic acid that can be selected or detected. These properties include, but are not limited to, a property affecting gene transcription (e.g., promoter strength or promoter recognition), a property affecting RNA processing (e.g., RNA splicing and RNA stability), a property affecting translation (e.g., regulation, binding of mRNA to ribosomal proteins).

The terms “thermally stable” and “thermostable” refer to glucoamylase variants of the present disclosure that retain a specified amount of enzymatic activity after exposure to a temperature over a given period of time under conditions prevailing during the hydrolysis of starch substrates, for example, while exposed to altered temperatures.

The term “enhanced stability” in the context of a property such as thermostability refers to a higher retained starch hydrolytic activity over time as compared to another reference (i.e., parent) glucoamylase.

The term “diminished stability” in the context of a property such as thermostability refers to a lower retained starch hydrolytic activity over time as compared to another reference glucoamylase.

The term “specific activity” is defined as the activity per mg of glucoamylase protein. In some embodiments, the activity for glucoamylase is determined by the ethanol assay described herein and expressed as the amount of glucose that is produced from the starch substrate. In some embodiments, the protein concentration can be determined using the Caliper assay described herein.

The terms “active” and “biologically active” refer to a biological activity associated with a particular protein. It follows that the biological activity of a given protein refers to any biological activity typically attributed to that protein by those skilled in the art. For example, an enzymatic activity associated with a glucoamylase is hydrolytic and, thus an active glucoamylase has hydrolytic activity.

The terms “polynucleotide” and “nucleic acid”, used interchangeably herein, refer to a polymeric form of nucleotides of any length, either ribonucleotides or deoxyribonucleotides. These terms include, but are not limited to, a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases.

As used herein, the terms “DNA construct,” “transforming DNA” and “expression vector” are used interchangeably to refer to DNA used to introduce sequences into a host cell or organism. The DNA may be generated in vitro by PCR or any other suitable technique(s) known to those in the art. The DNA construct, transforming DNA or recombinant expression cassette can be incorporated into a plasmid, chromosome, mitochondrial DNA, plastid DNA, virus, or nucleic acid fragment. Typically, the recombinant expression cassette portion of an expression vector, DNA construct or transforming DNA includes, among other sequences, a nucleic acid sequence to be transcribed and a promoter. In some embodiments, expression vectors have the ability to incorporate and express heterologous DNA fragments in a host cell.

As used herein, the term “vector” refers to a polynucleotide construct designed to introduce nucleic acids into one or more cell types. Vectors include cloning vectors, expression vectors, shuttle vectors, plasmids, cassettes, and the like.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction.

As used herein, the terms “transformed” and “stably transformed” refers to a cell that has a non-native (heterologous) polynucleotide sequence integrated into its genome or as an episomal plasmid that is maintained for at least two generations.

As used herein, the terms “selectable marker” and “selective marker” refer to a nucleic acid (e.g., a gene) capable of expression in host cells that allows for ease of selection of those hosts containing the vector. Typically, selectable markers are genes that confer antimicrobial resistance or a metabolic advantage on the host cell to allow cells containing the exogenous DNA to be distinguished from cells that have not received any exogenous sequence during the transformation.

As used herein, the term “promoter” refers to a nucleic acid sequence that functions to direct transcription of a downstream gene. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a preprotein that participates in the secretion of the polypeptide. Generally, “operably linked” means that the DNA sequences being linked are contiguous, and, in the case of a secretory leader, contiguous and in reading phase.

As used herein the term “gene” refers to a polynucleotide (e.g., a DNA segment), that encodes a polypeptide and includes regions preceding and following the coding regions, as well as intervening sequences (introns) between individual coding segments (exons).

As used herein, “ortholog” and “orthologous genes” refer to genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, the term “hybridization” refers to the process by which a strand of nucleic acid joins with a complementary strand through base pairing, as known in the art.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tₘ) of the nucleic acid binding complex or probe. For example, “maximum stringency” typically occurs at about Tₘ −5° C. (5° C. below the Tₘ of the probe); “high stringency” at about 5-10° C. below the Tₘ; “intermediate stringency” at about 10-20° C. below the Tₘ of the probe; and “low stringency” at about 20-25° C. below the Tₘ. Functionally, maximum stringency conditions may be used to identify sequences having strict identity or near-strict identity with the hybridization probe; while an intermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denaturated sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous or homologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

In an embodiment of the disclosure, mutated DNA sequences are generated with site saturation mutagenesis in at least one codon. In another embodiment, site saturation mutagenesis is performed for two or more codons. In a further embodiment, mutant DNA sequences have more than about 50%, about 55%, about 60%, about 65%, about 70%, about 75%, about 80%, about 85%, about 90%, about 95%, or about 98% identity with the parent sequence. In alternative embodiments, mutant DNA is generated in vivo using any known mutagenic procedure such as, for example, radiation, nitrosoguanidine, and the like. The desired DNA sequence is then isolated and used in the methods provided herein.

As used herein, “heterologous protein” refers to a protein or polypeptide that does not naturally occur in the host cell.

An enzyme is “over-expressed” in a host cell if the enzyme is expressed in the cell at a higher level than the level at which it is expressed in a corresponding wild-type cell.

The terms “protein” and “polypeptide” are used interchangeability herein. In the present disclosure and claims, the conventional one-letter and three-letter codes for amino acid residues are used. The 3-letter code for amino acids as defined in conformity with the IUPAC-IUB Joint Commission on Biochemical Nomenclature (JCBN). It is also understood that a polypeptide may be coded for by more than one nucleotide sequence due to the degeneracy of the genetic code.

Variants of the disclosure are described by the following nomenclature: [original amino acid residue/position/substituted amino acid residue]. For example, the substitution of leucine for arginine at position 76 is represented as R76L. When more than one amino acid is substituted at a given position, the substitution is represented as 1) Q172C, Q172D or Q172R; 2) Q172C, D, or R, or 3) Q172C/D/R. When a position suitable for substitution is identified herein without a specific amino acid suggested, it is to be understood that any amino acid residue may be substituted for the amino acid residue present in the position. Where a variant glucoamylase contains a deletion in comparison with other glucoamylases the deletion is indicated with “*”. For example, a deletion at position R76 is represented as R76*. A deletion of two or more consecutive amino acids is indicated for example as (76-78)*.

A “prosequence” is an amino acid sequence between the signal sequence and mature protein that is necessary for the secretion of the protein. Cleavage of the pro sequence will result in a mature active protein.

The term “signal sequence” or “signal peptide” refers to any sequence of nucleotides and/or amino acids that may participate in the secretion of the mature or precursor forms of the protein. This definition of signal sequence is a functional one, meant to include all those amino acid sequences encoded by the N-terminal portion of the protein gene, which participate in the effectuation of the secretion of protein. They are often, but not universally, bound to the N-terminal portion of a protein or to the N-terminal portion of a precursor protein. The signal sequence may be endogenous or exogenous. The signal sequence may be that normally associated with the protein (e.g., glucoamylase), or may be from a gene encoding another secreted protein.

The term “precursor” form of a protein or peptide refers to a mature form of the protein having a prosequence operably linked to the amino or carbonyl terminus of the protein. The precursor may also have a “signal” sequence operably linked, to the amino terminus of the prosequence. The precursor may also have additional polynucleotides that are involved in post-translational activity (e.g., polynucleotides cleaved therefrom to leave the mature form of a protein or peptide).

“Host strain” or “host cell” refers to a suitable host for an expression vector comprising DNA according to the present disclosure.

The terms “derived from” and “obtained from” refer to not only a glucoamylase produced or producible by a strain of the organism in question, but also a glucoamylase encoded by a DNA sequence isolated from such strain and produced in a host organism containing such DNA sequence. Additionally, the term refers to a glucoamylase that is encoded by a DNA sequence of synthetic and/or cDNA origin and that has the identifying characteristics of the glucoamylase in question.

A “derivative” within the scope of this definition generally retains the characteristic hydrolyzing activity observed in the wild-type, native or parent form to the extent that the derivative is useful for similar purposes as the wild-type, native or parent form. Functional derivatives of glucoamylases encompass naturally occurring, synthetically or recombinantly produced peptides or peptide fragments that have the general characteristics of the glucoamylases of the present disclosure.

The term “isolated” refers to a material that is removed from the natural environment if it is naturally occurring. A “purified” protein refers to a protein that is at least partially purified to homogeneity. In some embodiments, a purified protein is more than about 10% pure, about 20% pure, or about 30% pure, as determined by SDS-PAGE. Further aspects of the disclosure encompass the protein in a highly purified form (i.e., more than about 40% pure, about 60% pure, about 80% pure, about 90% pure, about 95% pure, about 97% pure, or about 99% pure), as determined by SDS-PAGE.

As used herein, the term, “combinatorial mutagenesis” refers to methods in which libraries of variants of a starting sequence are generated. In these libraries, the variants contain one or several mutations chosen from a predefined set of mutations. In addition, the methods provide means to introduce random mutations that were not members of the predefined set of mutations. In some embodiments, the methods include those set forth in U.S. Pat. No. 6,582,914, hereby incorporated by reference. In alternative embodiments, combinatorial mutagenesis methods encompass commercially available kits (e.g., QuikChange® Multisite, Stratagene, San Diego, Calif.).

As used herein, the term “library of mutants” refers to a population of cells that are identical in most of their genome but include different homologues of one or more genes. Such libraries can be used, for example, to identify genes or operons with improved traits.

As used herein the term “dry solids content (DS or ds)” refers to the total solids of a slurry in % on a dry weight basis.

As used herein, the term “initial hit” refers to a variant that was identified by screening a combinatorial consensus mutagenesis library. In some embodiments, initial hits have improved performance characteristics, as compared to the starting gene.

As used herein, the term “improved hit” refers to a variant that was identified by screening an enhanced combinatorial consensus mutagenesis library.

As used herein, the term “target property” refers to the property of the starting gene that is to be altered. It is not intended that the present disclosure be limited to any particular target property. However, in some embodiments, the target property is the stability of a gene product (e.g., resistance to denaturation, proteolysis or other degradative factors), while in other embodiments, the level of production in a production host is altered. Indeed, it is contemplated that any property of a starting gene will find use in the present disclosure. Other definitions of terms may appear throughout the specification.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Before the exemplary embodiments are described in more detail, it is to be understood that this disclosure is not limited to particular embodiments described, as such may, of course, vary. Although any methods and materials similar or equivalent to those described herein can be used in the practice or testing of the present disclosure, exemplary methods and materials are now described.

As used herein and in the appended claims, the singular forms “a”, “an”, and “the” include plural referents unless the context clearly dictates otherwise. Thus, for example, reference to “a gene” includes a plurality of such candidate agents and reference to “the cell” includes reference to one or more cells and equivalents thereof known to those skilled in the art, and so forth.

The publications discussed herein are provided solely for their disclosure prior to the filing date of the present application. Nothing herein is to be construed as an admission that the present disclosure is not entitled to antedate such publication by virtue of prior invention.

1.2. Abbreviations

GA glucoamylase

GAU glucoamylase unit

wt % weight percent

° C. degrees Centigrade

rpm revolutions per minute

H₂O water

dH₂O deionized water

dIH₂O deionized water, Milli-Q filtration

aa or AA amino acid

bp base pair

kb kilobase pair

kD kilodaltons

g or gm grams

μS micrograms

mg milligrams

μl and μL microliters

ml and mL milliliters

mm millimeters

μm micrometer

M molar

mM millimolar

micromolar

U units

V volts

MW molecular weight

MWCO molecular weight cutoff

sec(s) or s(s) second/seconds

min(s) or m(s) minute/minutes

hr(s) or h(s) hour/hours

DO dissolved oxygen

ABS Absorbance

EtOH ethanol

PSS physiological salt solution

m/v mass/volume

MTP microtiter plate

N Normal

DP1 monosaccharides

DP2 disaccharides

DP>3 oligosaccharides, sugars having a degree of polymerization greater than 3

ppm parts per million

SBD starch binding domain

CD catalytic domain

PCR polymerase chain reaction

WT wild-type

2. Parent Glucoamylases

In some embodiments, the present disclosure provides a glucoamylase variant. The glucoamylase variant is a variant of a parent glucoamylase, which may comprise both a catalytic domain and a starch binding domain. In some embodiments, the parent glucoamylase comprises a catalytic domain having an amino acid sequence as illustrated in SEQ ID NO: 1, 2, 3, 5, 6, 7, 8 or 9 or having an amino acid sequence displaying at least about 80%, about 85%, about 90%, about 95%, about 97%, about 99%, or about 99.5% sequence identity with one or more of the amino acid sequences illustrated in SEQ ID NO: 1, 2, 3, 5, 6, 7, 8, or 9. In yet other embodiments, the parent glucoamylase comprises a catalytic domain encoded by a DNA sequence that hybridizes under medium, high, or stringent conditions with a DNA encoding the catalytic domain of a glucoamylase having one of the amino acid sequences of SEQ ID NO: 1, 2 or 3.

In some embodiments, the parent glucoamylase comprises a starch binding domain having an amino acid sequence as illustrated in SEQ ID NO 1, 2, 11, 385, 386, 387, 388, 389, or 390, or having an amino acid sequence displaying at least about 80%, about 85%, about 90%, about 95%, about 97%, about 99%, or about 99.5% sequence identity with one or more of the amino acid sequence illustrated in SEQ ID NO 1, 2, 11, 385, 386, 387, 388, 389, or 390. In yet other embodiments, the parent glucoamylase comprises a starch binding domain encoded by a DNA sequence that hybridizes under medium, high, or stringent conditions with a DNA encoding the starch binding domain of a glucoamylase having one of the amino acid sequences of SEQ ID NO: 1, 2, or 11.

Predicted structure and known sequences of glucoamylases are conserved among fungal species (Coutinho et al., 1994, Protein Eng., 7:393-400 and Coutinho et al., 1994, Protein Eng., 7: 749-760). In some embodiments, the parent glucoamylase is a filamentous fungal glucoamylase. In some embodiments, the parent glucoamylase is obtained from a Trichoderma strain (e.g., T. reesei, T. longibrachiatum, T. strictipilis, T. asperellum, T. konilangbra and T. hazianum), an Aspergillus strain (e.g. A. niger, A. nidulans, A. kawachi, A. awamori and A. orzyae), a Talaromyces strain (e.g. T. emersonii, T. thermophilus, and T. duponti), a Hypocrea strain (e.g. H. gelatinosa, H. orientalis, H. vinosa, and H. citrina), a Fusarium strain (e.g., F. oxysporum, F. roseum, and F. venenatum), a Neurospora strain (e.g., N. crassa) and a Humicola strain (e.g., H. grisea, H. insolens and H. lanuginose), a Penicillium strain (e.g., P. notatum or P. chrysogenum), or a Saccharomycopsis strain (e.g., S. fibuligera).

In some embodiments, the parent glucoamylase may be a bacterial glucoamylase. For example, the polypeptide may be obtained from a gram-positive bacterial strain such as Bacillus (e.g., B. alkalophilus, B. amyloliquefaciens, B. lentus, B. lichenifonnis, B. stearothermophilus, B. subtilis and B. thuringiensis) or a Streptomyces strain (e.g., S. lividans).

In some embodiments, the parent glucoamylase will comprise a catalytic domain having at least about 80%, about 85%, about 90%, about 93%, about 95%, about 97%, about 98%, or about 99% sequence identity with the catalytic domain of the TrGA amino acid sequence of SEQ ID NO: 3.

In other embodiments, the parent glucoamylase will comprise a catalytic domain having at least about 90%, about 93%, about 95%, about 96%, about 97%, about 98%, or about 99% sequence identity with the catalytic domain of the Aspergillus parent glucoamylase of SEQ ID NO: 5 or SEQ ID NO: 6.

In yet other embodiments, the parent glucoamylase will comprise a catalytic domain having at least about 90%, about 95%, about 97%, or about 99% sequence identity with the catalytic domain of the Humicola grisea (HgGA) parent glucoamylase of SEQ ID NO: 8.

In some embodiments, the parent glucoamylase will comprise a starch binding domain having at least about 80%, about 85%, about 90%, about 95%, about 97%, or about 98% sequence identity with the starch binding domain of the TrGA amino acid sequence of SEQ ID NO: 1, 2, or 11.

In other embodiments, the parent glucoamylase will comprise a starch binding domain having at least about 90%, about 95%, about 97%, or about 99% sequence identity with the catalytic domain of the Humicola grisea (HgGA) glucoamylase of SEQ ID NO: 385.

In other embodiments, the parent glucoamylase will comprise a starch binding domain having at least about 90%, about 95%, about 97%, or about 99% sequence identity with the catalytic domain of the Thielavia terrestris (TtGA) glucoamylase of SEQ ID NO: 390.

In other embodiments, the parent glucoamylase will comprise a starch binding domain having at least about 90%, about 95%, about 97%, or about 99% sequence identity with the catalytic domain of the Thermomyces lanuginosus (ThGA) glucoamylase of SEQ ID NO: 386.

In other embodiments, the parent glucoamylase will comprise a starch binding domain having at least about 90%, about 95%, about 97%, or about 99% sequence identity with the catalytic domain of the Talaromyces emersoniit (TeGA) glucoamylase of SEQ ID NO: 387.

In yet other embodiments, the parent glucoamylase will comprise a starch binding domain having at least about 90%, about 93%, about 95%, about 96%, about 97%, about 98%, or about 99% sequence identity with the starch binding domain of the Aspergillus parent glucoamylase of SEQ ID NO: 388 or 389.

In some embodiments, the parent glucoamylase will have at least about 80%, about 85%, about 88%, about 90%, about 93%, about 95%, about 96%, about 97%, about 98%, or about 99% sequence identity with the TrGA amino acid sequence of SEQ ID NO: 1 or 2.

In further embodiments, a Trichoderma glucoamylase homologue will be obtained from a Trichoderma or Hypocrea strain. Some typical Trichoderma glucoamylase homologues are described in U.S. Pat. No. 7,413,887 and reference is made specifically to amino acid sequences set forth in SEQ ID NOs: 17-22 and 43-47 of the reference.

In some embodiments, the parent glucoamylase is TrGA comprising the amino acid sequence of SEQ ID NO: 2, or a Trichoderma glucoamylase homologue having at least about 80%, about 85%, about 88%, about 90%, about 93%, about 95%, about 96%, about 97%, about 98%, or about 99% sequence identity to the TrGA sequence (SEQ ID NO: 2).

A parent glucoamylase can be isolated and/or identified using standard recombinant DNA techniques. Any standard techniques can be used that are known to the skilled artisan. For example, probes and/or primers specific for conserved regions of the glucoamylase can be used to identify homologs in bacterial or fungal cells (the catalytic domain, the active site, etc.). Alternatively, degenerate PCR can be used to identify homologues in bacterial or fungal cells. In some cases, known sequences, such as in a database, can be analyzed for sequence and/or structural identity to one of the known glucoamylases, including SEQ ID NO: 2, or a known starch binding domains, including SEQ ID NO: 11. Functional assays can also be used to identify glucoamylase activity in a bacterial or fungal cell. Proteins having glucoamylase activity can be isolated and reverse sequenced to isolate the corresponding DNA sequence. Such methods are known to the skilled artisan.

3. Glucoamylase Structural Homology

The central dogma of molecular biology is that the sequence of DNA encoding a gene for a particular enzyme, determines the amino acid sequence of the protein, this sequence in turn determines the three-dimensional folding of the enzyme. This folding brings together disparate residues that create a catalytic center and substrate binding surface and this results in the high specificity and activity of the enzymes in question.

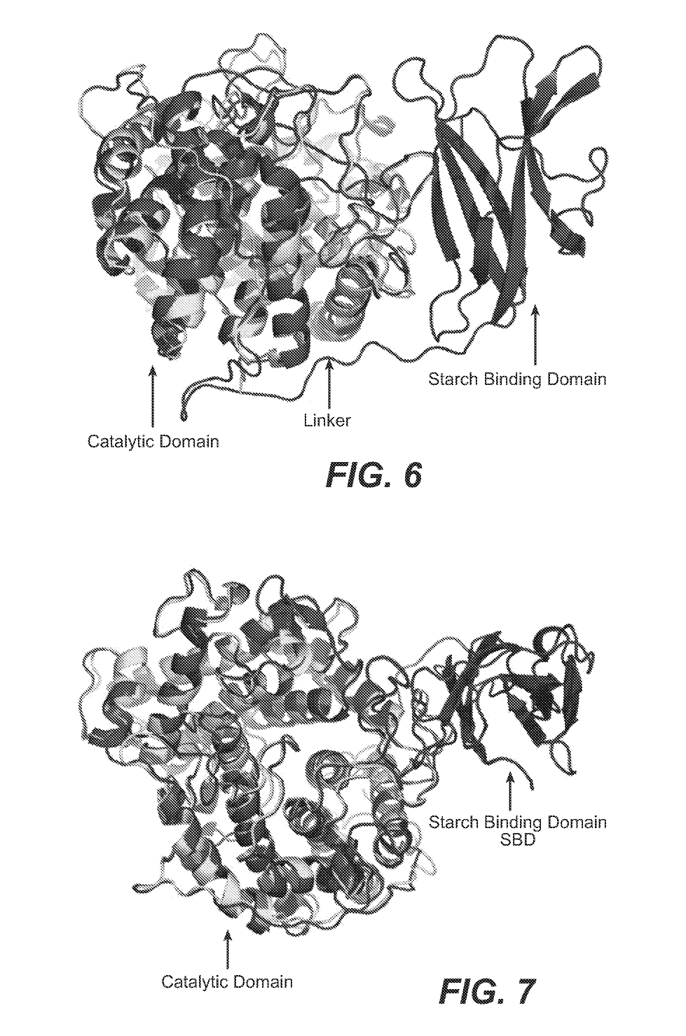

Glucoamylases consist of as many as three distinct structural domains, a catalytic domain of approximately 450 residues that is structurally conserved in all glucoamylases, generally followed by a linker region consisting of between 30 and 80 residues that are connected to a starch binding domain of approximately 100 residues. The structure of the Trichoderma reesei glucoamylase with all three regions intact was determined to 1.8 Angstrom resolution herein (see Table 9 and Example 9). Using the coordinates (see Table 9), the structure was aligned with the coordinates of the catalytic domain of the glucoamylase from Aspergillus awamori strain X100 that was determined previously (Aleshin, A. E., Hoffman, C., Firsov, L. M., and Honzatko, R. B. Refined crystal structures of glucoamylase from Aspergillus awamori var. X100. J. Mol. Biol. 238: 575-591 (1994)). The Aspergillus awamori crystal structure only included the catalytic domain. As seen in FIGS. 6-7, the structure of the catalytic domains overlap very closely, and it is possible to identify equivalent residues based on this structural superposition. It is believed that all glucoamylases share the basic structure depicted in FIGS. 6-7.

FIG. 6 is a comparison of the three dimensional structures of the Trichoderma reesei glucoamylase (black) of SEQ ID NO: 2 and of Aspergillus awamorii glucoamylase (grey) viewed from the side. In this view, the relationship between the catalytic domain and the linker region and the starch binding domain can be seen.

FIG. 7 is a comparison of the three dimensional structures of the Trichoderma reesei glucoamylase (black) of SEQ ID NO: 2 and of Aspergillus awamorii glucoamylase (grey) viewed from the top. The glucoamylases shown here and indeed all known glucoamylases to date share this structural homology. The conservation of structure correlates with the conservation of activity and a conserved mechanism of action for all glucoamylases. Given this high homology, changes resulting from site specific variants of the Trichoderma glucoamylase resulting in altered functions would also have similar structural and therefore functional consequences in other glucoamylases. Therefore, the teachings of which variants result in desirable benefits can be applied to other glucoamylases.

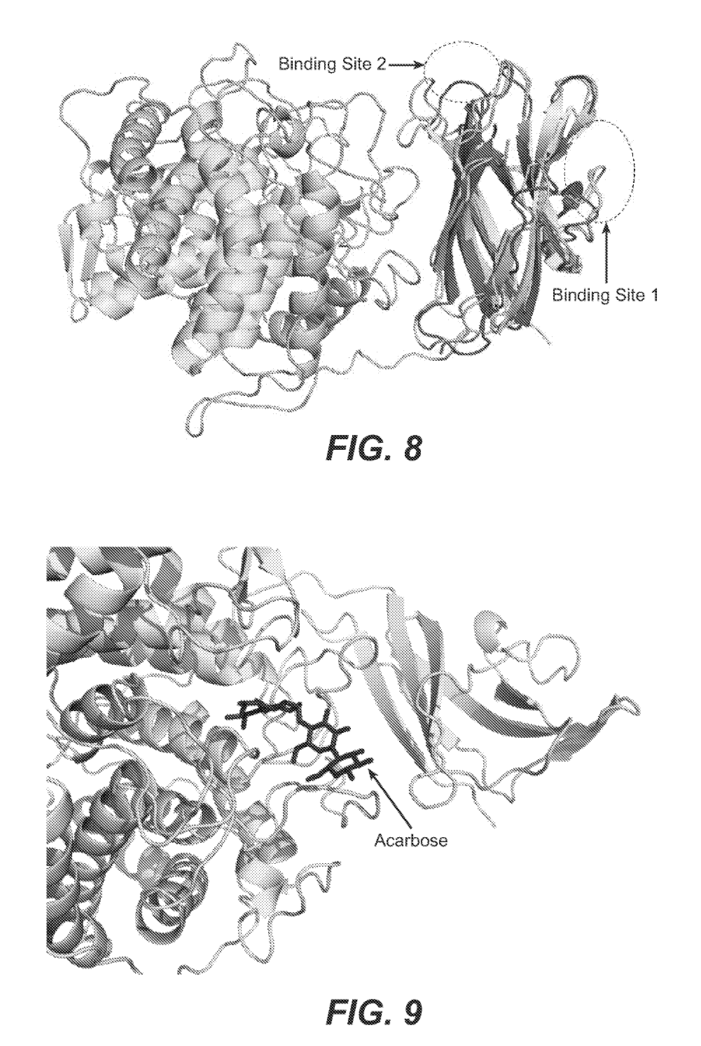

A further crystal structure was produced using the coordinates in Table 9 for the Starch Binding Domain (SBD). The SBD for TrGA was aligned with the SBD for A. niger. As shown in FIG. 8, the structure of the A. niger and TrGA SBDs overlaps very closely. It is believed that while all starch binding domains share at least some of the basic structure depicted in FIG. 8, some SBDs are more structurally similar than others. For example, the TrGA SBD can be classified as within the carbohydrate binding module 20 family within the CAZY database (cazy.org). The CAZY database describes the families of structurally-related catalytic and carbohydrate-binding modules (or functional domains) of enzymes that degrade, modify, or create glycosidic bonds. Given a high structural homology, site specific variants of the TrGA SBD resulting in altered function would also have similar structural and therefore functional consequences in other glucoamylases having SBDs with similar structure to that of the TrGA SBD, particularly those classified within the carbohydrate binding module 20 family. Thus, the teachings of which variants result in desirable benefits can be applied to other SBDs having structural similarity.

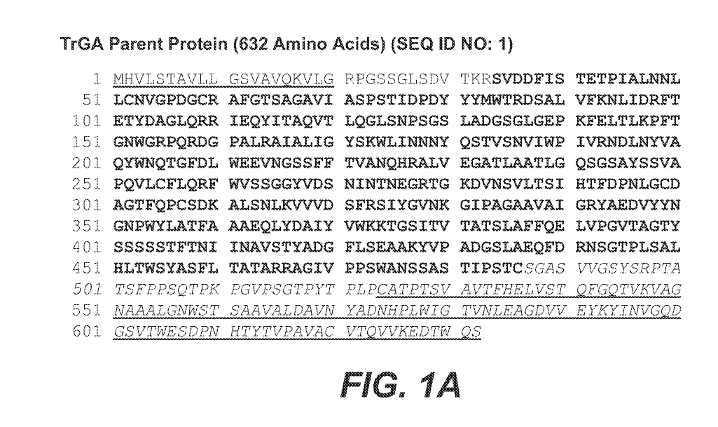

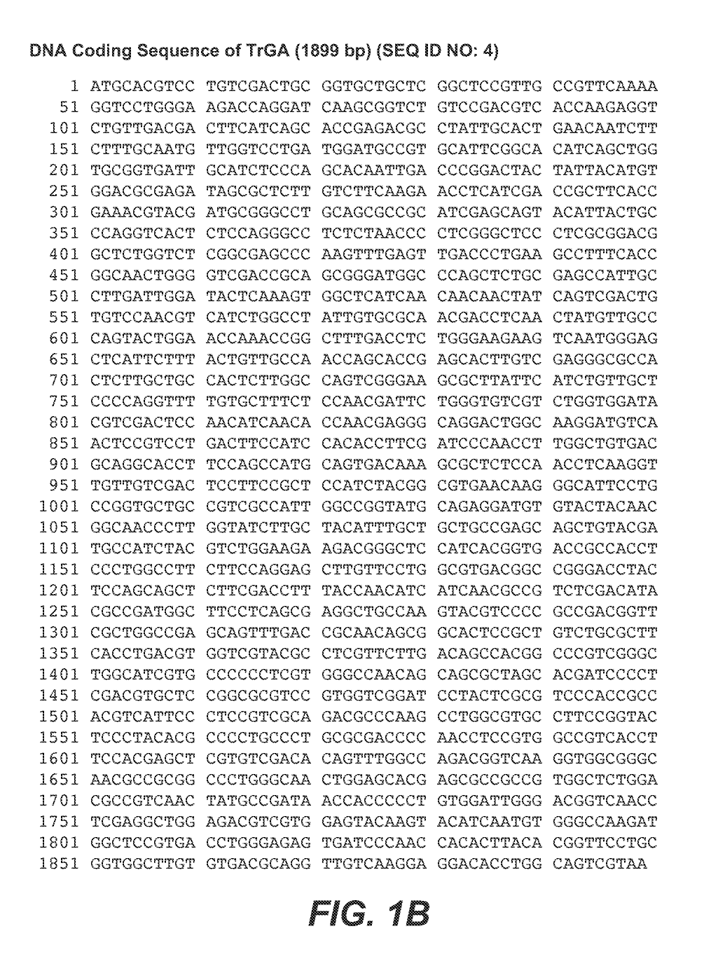

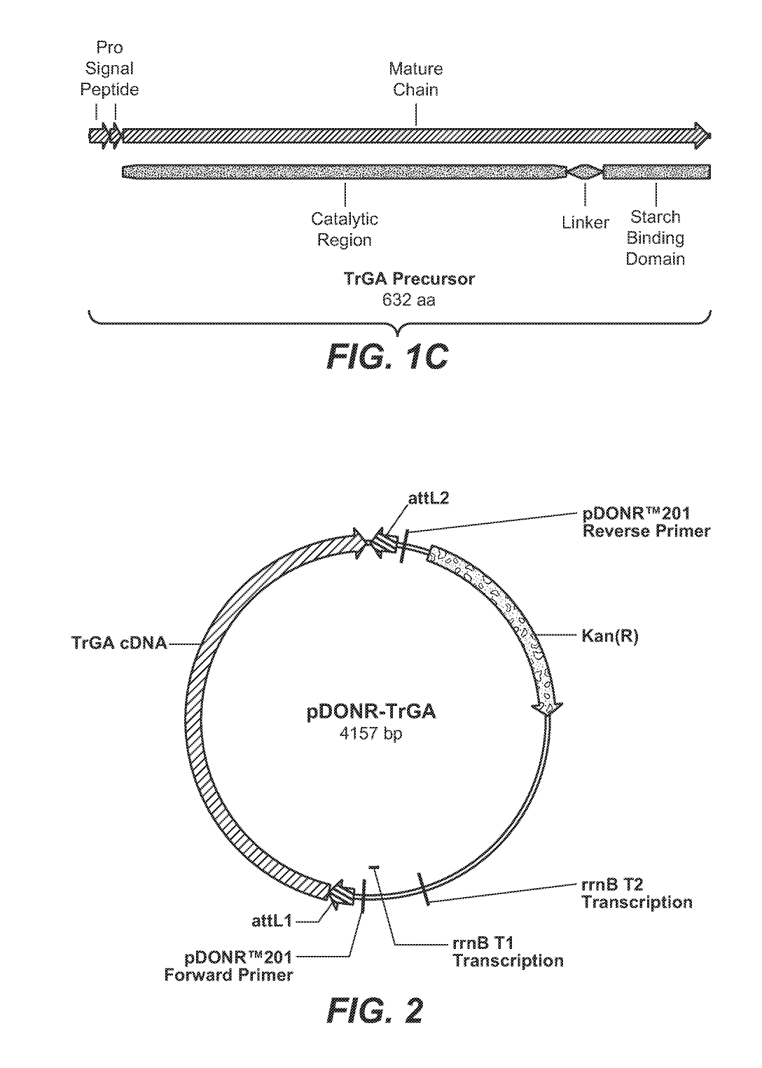

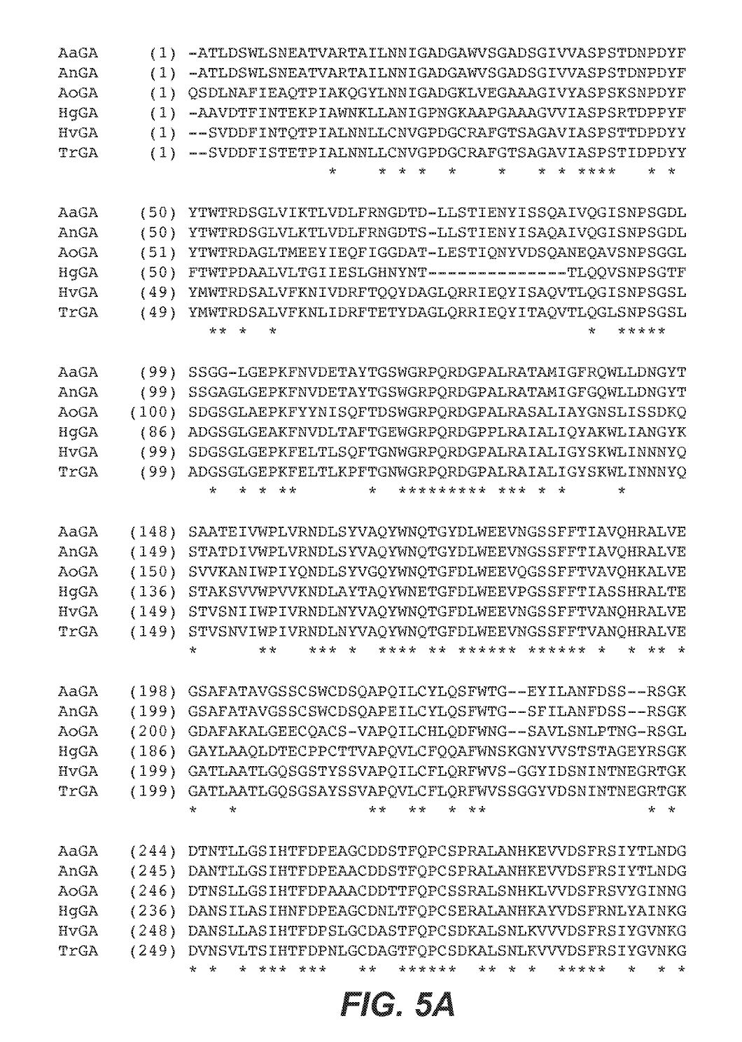

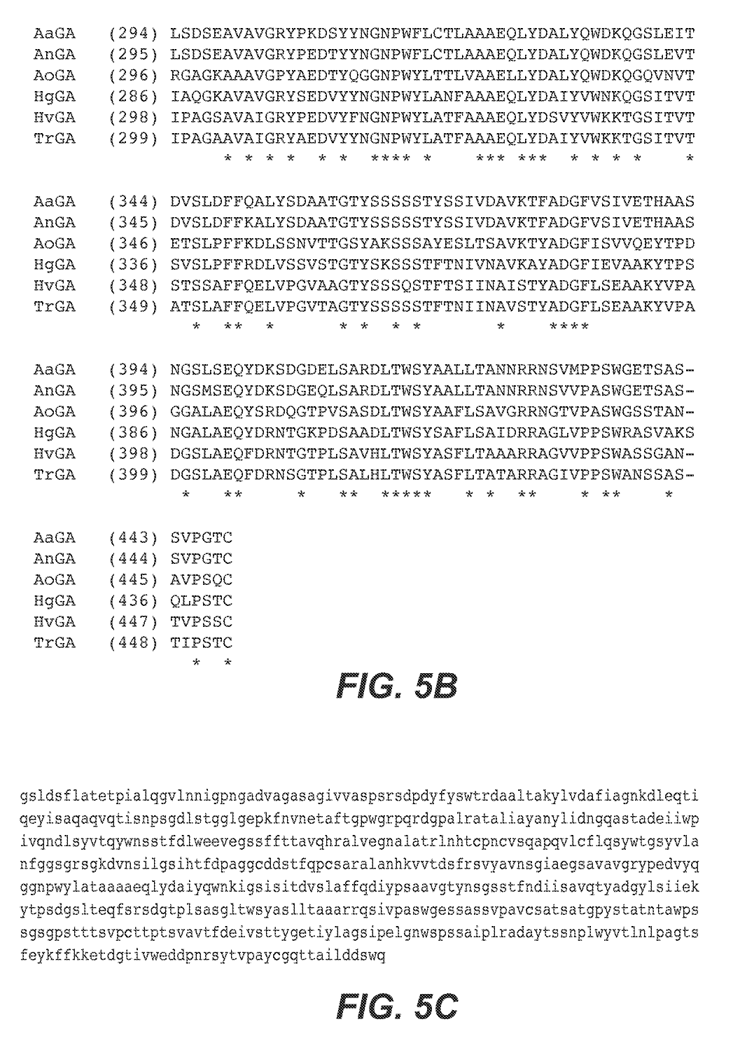

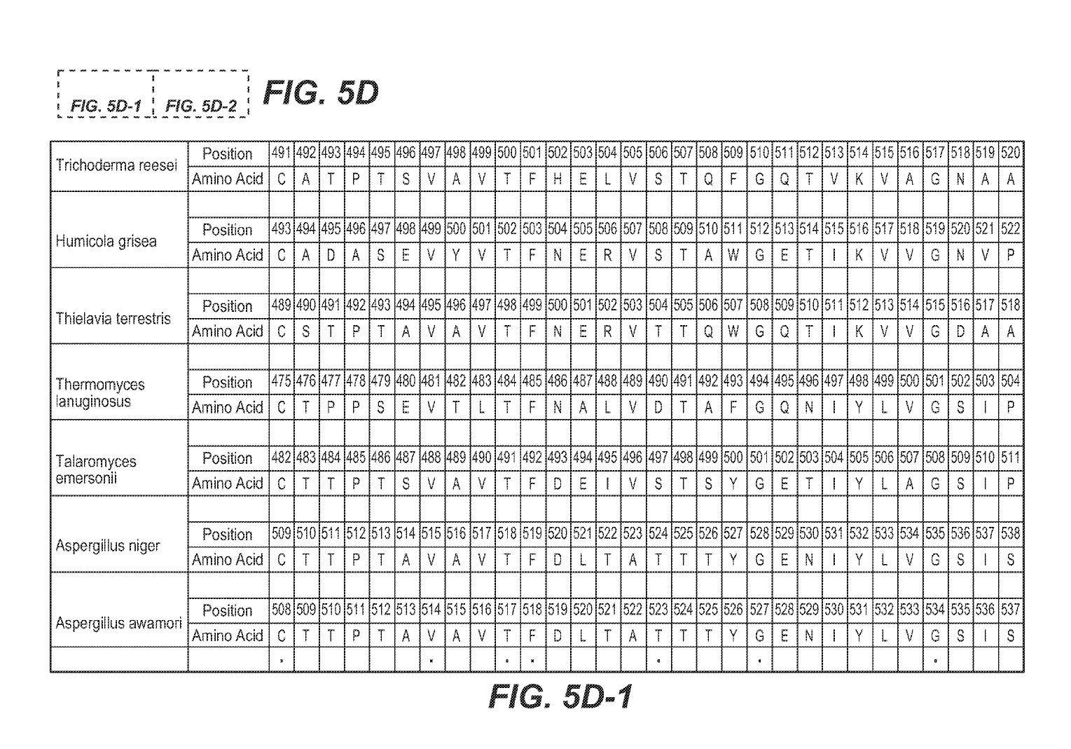

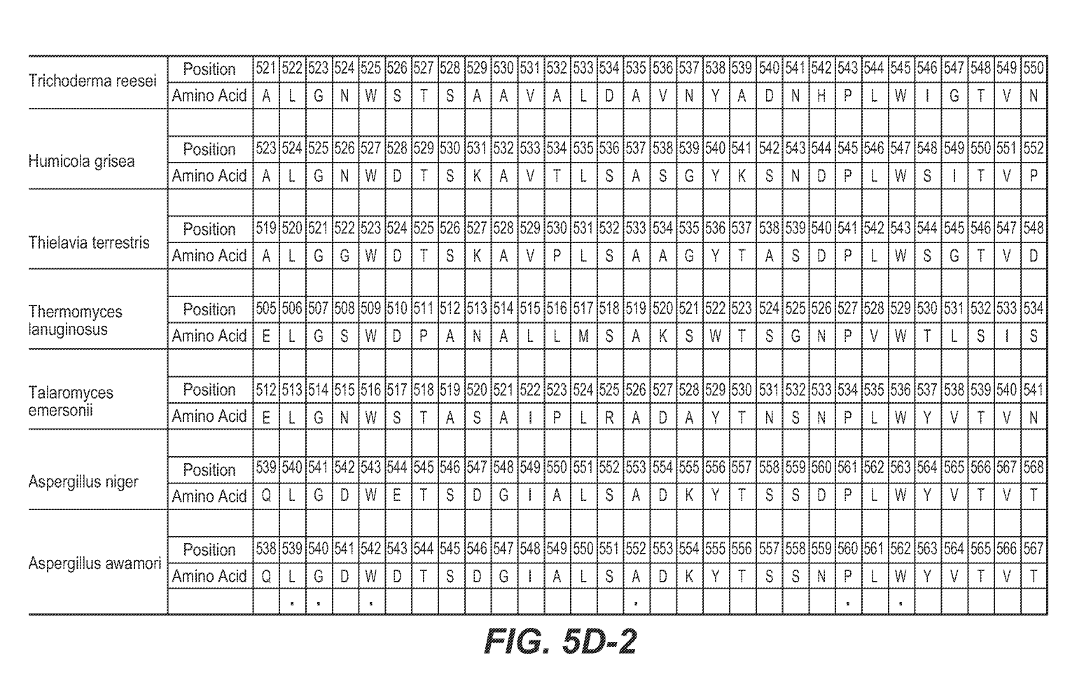

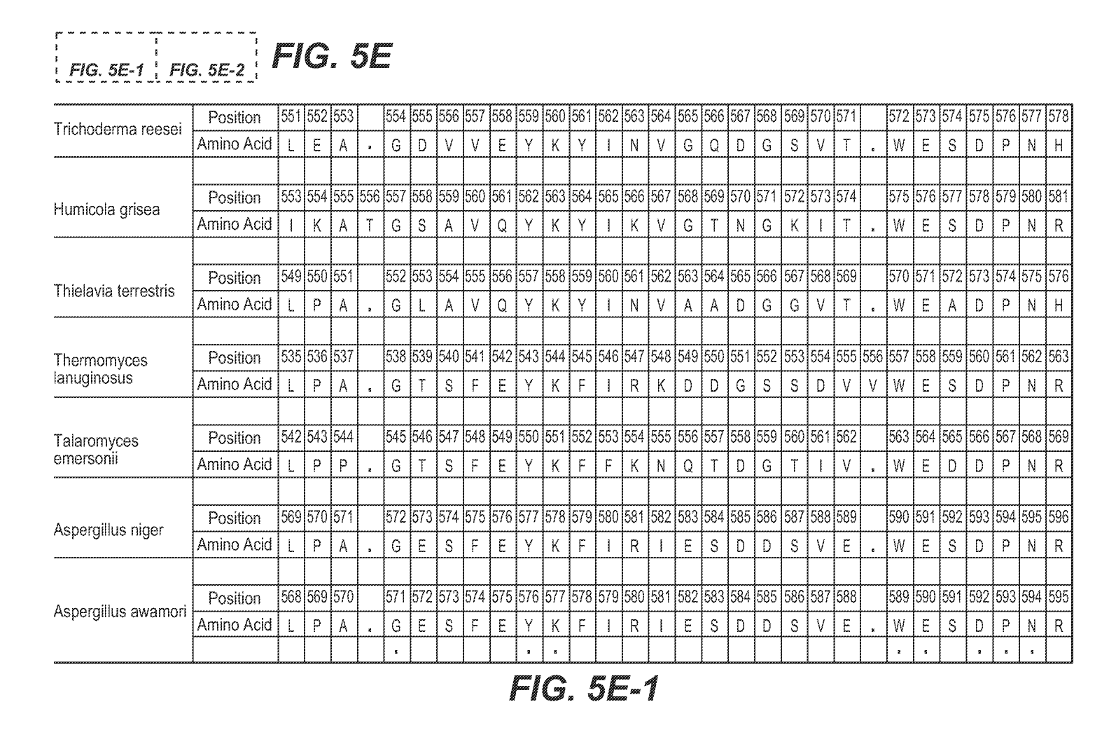

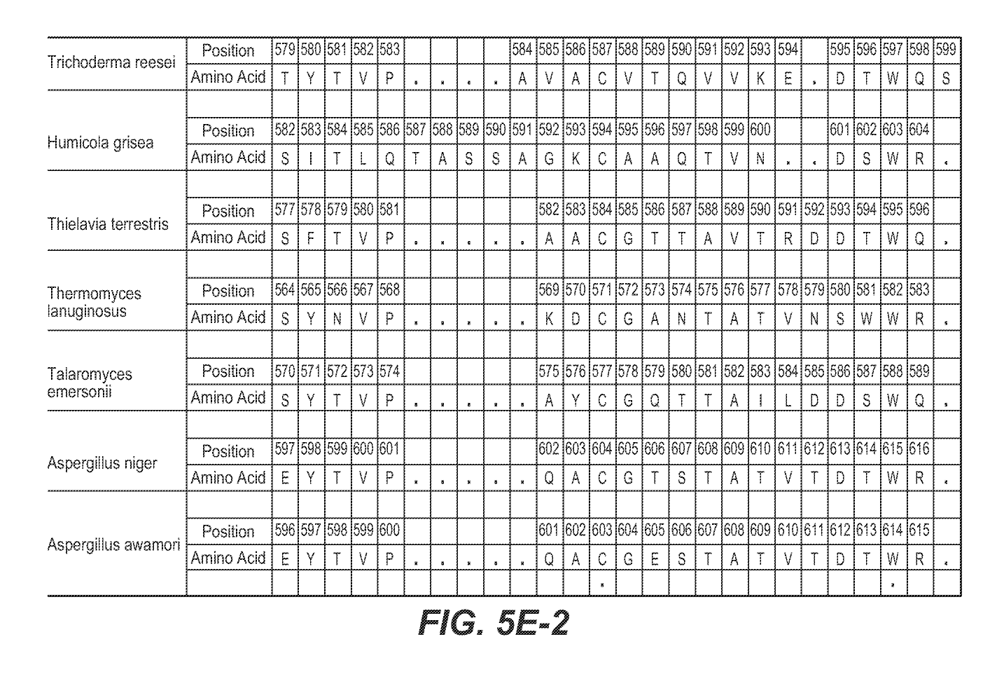

Thus, the amino acid position numbers discussed herein refer to those assigned to the mature Trichoderma reesei glucoamylase sequence presented in FIG. 1 (SEQ ID NO: 2). The present disclosure, however, is not limited to the variants of Trichoderma glucoamylase, but extends to glucoamylases containing amino acid residues at positions that are “equivalent” to the particular identified residues in Trichoderma reesei glucoamylase (SEQ ID NO: 2). In some embodiments of the present disclosure, the parent glucoamylase is a Talaromyces GA and the substitutions are made at the equivalent amino acid residue positions in Talaromyces glucoamylase (see e.g., SEQ ID NO: 12) as those described herein. In other embodiments, the parent glucoamylase comprises SEQ ID NOs: 5-9 (see FIGS. 5A and 5B). In further embodiments, the parent glucoamylase is a Penicillium glucoamylase, such as Penicillium chrysogenum (see e.g., SEQ ID NO: 13).

“Structural identity” determines whether the amino acid residues are equivalent. Structural identity is a one-to-one topological equivalent when the two structures (three dimensional and amino acid structures) are aligned. A residue (amino acid) position of a glucoamylase is “equivalent” to a residue of T. reesei glucoamylase if it is either homologous (i.e., corresponding in position in either primary or tertiary structure) or analogous to a specific residue or portion of that residue in T. reesei glucoamylase (having the same or similar functional capacity to combine, react, or interact chemically).

In order to establish identity to the primary structure, the amino acid sequence of a glucoamylase can be directly compared to Trichoderma reesei glucoamylase primary sequence and particularly to a set of residues known to be invariant in glucoamylases for which sequence is known. For example, FIGS. 5A and 5B herein show the conserved residues between glucoamylases. FIGS. 5D and 5E show an alignment of starch binding domains from various glucoamylases. After aligning the conserved residues, allowing for necessary insertions and deletions in order to maintain alignment (i.e. avoiding the elimination of conserved residues through arbitrary deletion and insertion), the residues equivalent to particular amino acids in the primary sequence of Trichoderma reesei glucoamylase are defined. Alignment of conserved residues typically should conserve 100% of such residues. However, alignment of greater than about 75% or as little as about 50% of conserved residues is also adequate to define equivalent residues. Further, the structural identity can be used in combination with the sequence identity to identify equivalent residues.

For example, in FIGS. 5A and 5B, the catalytic domains of glucoamylases from six organisms are aligned to provide the maximum amount of homology between amino acid sequences. A comparison of these sequences shows that there are a number of conserved residues contained in each sequence as designated by an asterisk. These conserved residues, thus, may be used to define the corresponding equivalent amino acid residues of Trichoderma reesei glucoamylase in other glucoamylases such as glucoamylase from Aspergillus niger. Similarly, FIGS. 5D and 5E show the starch binding domains of glucoamylases from seven organisms aligned to identify equivalent residues.

Structural identity involves the identification of equivalent residues between the two structures. “Equivalent residues” can be defined by determining homology at the level of tertiary structure (structural identity) for an enzyme whose tertiary structure has been determined by X-ray crystallography. Equivalent residues are defined as those for which the atomic coordinates of two or more of the main chain atoms of a particular amino acid residue of the Trichoderma reesei glucoamylase (N on N, CA on CA, C on C and O on O) are within 0.13 nm and optionally 0.1 nm after alignment. Alignment is achieved after the best model has been oriented and positioned to give the maximum overlap of atomic coordinates of non-hydrogen protein atoms of the glucoamylase in question to the Trichoderma reesei glucoamylase. The best model is the crystallographic model giving the lowest R factor for experimental diffraction data at the highest resolution available.

R factor

Equivalent residues that are functionally analogous to a specific residue of Trichoderma reesei glucoamylase are defined as those amino acids of the enzyme that may adopt a conformation such that they either alter, modify or contribute to protein structure, substrate binding or catalysis in a manner defined and attributed to a specific residue of the Trichoderma reesei glucoamylase. Further, they are those residues of the enzyme (for which a tertiary structure has been obtained by X-ray crystallography) that occupy an analogous position to the extent that, although the main chain atoms of the given residue may not satisfy the criteria of equivalence on the basis of occupying a homologous position, the atomic coordinates of at least two of the side chain atoms of the residue lie with 0.13 nm of the corresponding side chain atoms of Trichoderma reesei glucoamylase. The coordinates of the three dimensional structure of Trichoderma reesei glucoamylase are set forth in Table 9 and can be used as outlined above to determine equivalent residues on the level of tertiary structure.

Some of the residues identified for substitution are conserved residues whereas others are not. In the case of residues that are not conserved, the substitution of one or more amino acids is limited to substitutions that produce a variant that has an amino acid sequence that does not correspond to one found in nature. In the case of conserved residues, such substitutions should not result in a naturally-occurring sequence.

4. Glucoamylase Variants

The variants according to the disclosure include at least one substitution, deletion or insertion in the amino acid sequence of a parent glucoamylase that makes the variant different in sequence from a parent glucoamylase. In some embodiments, the variants of the disclosure will have at least about 20%, about 40%, about 50%, about 60%, about 70%, about 80%, about 85%, about 90%, about 95%, about 97%, or about 100% of the glucoamylase activity as that of the TrGA (SEQ ID NO: 2), a parent glucoamylase that has at least 80% sequence identity to TrGA (SEQ ID NO: 2). In some embodiments, the variants according to the disclosure will comprise a substitution, deletion or insertion in at least one amino acid position of the parent TrGA (SEQ ID NO: 2), or in an equivalent position in the sequence of another parent glucoamylase having at least about 80%, about 85%, about 90%, about 95%, about 97%, about 98%, or about 99% sequence identity to the TrGA sequence (SEQ ID NO: 2).

In other embodiments, the variant according to the disclosure will comprise a substitution, deletion or insertion in at least one amino acid position of a fragment of the parent TrGA, wherein the fragment comprises the catalytic domain of the TrGA sequence (SEQ ID NO: 3) or in an equivalent position in a fragment comprising the catalytic domain of a parent glucoamylase having at least about 80%, about 85%, about 90%, about 95%, about 97%, about 98%, or about 99% sequence identity to the catalytic-domain-containing fragment of the SEQ ID NO: 3, 5, 6, 7, 8, or 9. In some embodiments, the fragment will comprise at least about 400, about 425, about 450, or about 500 amino acid residues of TrGA catalytic domain (SEQ ID NO: 3).

In other embodiments, the variant according to the disclosure will comprise a substitution, deletion or insertion in at least one amino acid position of a fragment of the parent TrGA, wherein the fragment comprises the starch binding domain of the TrGA sequence (SEQ ID NO: 11) or in an equivalent position in a fragment comprising the starch binding domain of a parent glucoamylase having at least about 80%, about 85%, about 90%, about 95%, about 97%, about 98%, or about 99% sequence identity to the starch-binding-domain-containing fragment of SEQ ID NO: 11, 385, 386, 387, 388, 389, and 390. In some embodiments, the fragment will comprise at least about 40, about 50, about 60, about 70, about 80, about 90, about 100, or about 109 amino acid residues of TrGA starch binding domain (SEQ ID NO: 11).

In some embodiments, when the parent glucoamylase includes a catalytic domain, a linker region, and a starch binding domain, the variant will comprise a substitution, deletion or insertion in at least one amino acid position of a fragment comprising part of the linker region. In some embodiments, the variant will comprise a substitution deletion, or insertion in the amino acid sequence of a fragment of the TrGA sequence (SEQ ID NO: 2).

Structural identity with reference to an amino acid substitution means that the substitution occurs at the equivalent amino acid position in the homologous glucoamylase or parent glucoamylase. The term equivalent position means a position that is common to two parent sequences that is based on an alignment of the amino acid sequence of the parent glucoamylase in question as well as alignment of the three-dimensional structure of the parent glucoamylase in question with the TrGA reference glucoamylase amino acid sequence and three-dimensional sequence. For example, with reference to FIG. 5A, position 24 in TrGA (SEQ ID NO: 2 or 3) is D24 and the equivalent position for Aspergillus niger (SEQ ID NO: 6) is position D25, and the equivalent position for Aspergillus oryzea (SEQ ID NO: 7) is position D26. See FIGS. 6 and 7 for an exemplary alignment of the three-dimensional sequence.

In some embodiments, the glucoamylase variant will include at least one substitution in the amino acid sequence of a parent. In further embodiments, the variant may have more than one substitution. For example, the variant may have 1, 2, 3, 4, 5, 6, 7, 8, 9, 10, 15, 20, or 25 amino acid substitutions, deletions, or insertions as compared to a corresponding parent glucoamylase.

In some embodiments, a glucoamylase variant comprises a substitution, deletion or insertion, and typically a substitution in at least one amino acid position in a position corresponding to the regions of non-conserved amino acids as illustrated in FIGS. 5A, 5B, 5D, and 5E (e.g., amino acid positions corresponding to those positions that are not designated by “*” in FIGS. 5A, 5B, 5D, and 5E).

While the variants may have substitutions in any position of the mature protein sequence (SEQ ID NO: 2), in some embodiments, a glucoamylase variant comprises one or more substitutions in the following positions in the amino acid sequence set forth in SEQ ID NO: 2: 10, 14, 15, 23, 42, 43, 44, 59, 60, 61, 65, 67, 68, 72, 73, 97, 98, 99, 102, 110, 113, 114, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 294, 300, 301, 303, 311, 338, 342, 344, 346, 349, 359, 361, 364, 375, 379, 382, 390, 391, 393, 394, 410, 417, 418, 430, 431, 433, 436, 442, 444, 448, 451, 493, 494, 495, 502, 503, 508, 511, 518, 519, 520, 527, 531, 535, 536, 537, 539, 563, or 577, or in an equivalent position in a parent glucoamylase. In some embodiments, the parent glucoamylase will have at least about 50%, about 60%, about 70%, about 80%, about 90%, about 95%, about 96%, about 97%, about 98%, or about 99% sequence identity with SEQ ID NO: 2. In other embodiments, the parent glucoamylase will be a Trichoderma glucoamylase homologue. In some embodiments, the variant will have altered properties. In some embodiments, the parent glucoamylase will have structural identity with the glucoamylase of SEQ ID NO: 2.

In some embodiments, the glucoamylase variant comprises one or more substitutions in the following positions in the amino acid sequence set forth in SEQ ID NO: 2: T10, L14, N15, P23, T42, I43, D44, P45, D46, F59, K60, N61, T67, E68, A72, G73, S97, L98, A99, S102, K108, E110, L113, K114, R122, Q124, R125, I133, K140, N144, N145, Y147, S152, N153, N164, F175, N182, A204, T205, S214, V216, Q219, W228, V229, S230, S231, D236, I239, N240, T241, N242, G244, N263, L264, G265, A268, G269, D276, V284, S291, G294, P300, A301, A303, Y310, A311, D313, Y316, V338, T342, S344, T346, A349, V359, G361, A364, T375, N379, S382, S390, E391, A393, K394, R408, S410, S415, L417, H418, T430, A431, R433, 1436, A442, N443, S444, T448, S451, T493, P494, T495, H₅₀₂, E503, Q508, Q511, N518, A519, A520, T527, V531, A535, V536, N537, A539, N563, and N577 or an equivalent position in parent glucoamylase (e.g., a Trichoderma glucoamylase homologue). In some embodiments, the variant will have altered properties as compared to the parent glucoamylase. In further embodiments, the variant of a glucoamylase parent comprises at least one of the following substitutions in the following positions in an amino acid sequence set forth in SEQ ID NO: 2: T10S, T42V, I43Q/R, D44R/C, N61I, T67M, E68C/M, A72Y, G73F/W, S97N, S102A/M/R, K114M/Q, I133T/V, N145I, N153A/D/E/M/S/V, T205Q, Q219S, W228A/F/H/M/V, V229I/L, S230C/F/G/L/N/Q/R, S231L/V, D236R, I239V/Y, N263P, L264D/K, A268C/D/G/K, S291A/F/H/M/T, G294C, A301P/R, V338I/N/Q, T342V, S344M/P/Q/R/V, G361D/E/F/I/L/M/P/S/W/Y, A364D/E/F/G/K/L/M/R/S/T/V/W, T375N, K394S, L417K/R/V, T430A/K, A431I/L/Q, R433c/E/G/L/N/S/V/Y, I436H, T451K, T495K/M/S, E503A/C/V, Q508R, Q511H, A519I/K/R/Y, A520C/L/P, V531L, A535K/N/P/R, V536M, A539E/R/S, N563C/E/I/K/K/Q/T/V, or N577K/P/R, or a substitution in an equivalent position in a parent glucoamylase.

Glucoamylase variants of the disclosure may also include chimeric or hybrid glucoamylases with, for example a starch binding domain (SBD) from one glucoamylase and a catalytic domain and linker from another. For example, a hybrid glucoamylase can be made by swapping the SBD from AnGA (SEQ ID NO: 6) with the SBD from TrGA (SEQ ID NO: 2), making a hybrid with the AnGA SBD and the TrGA catalytic domain and linker. Alternatively, the SBD and linker from AnGA can be swapped for the SBD and linker of TrGA.

In some aspects, the variant glucoamylase exhibits altered thermostability as compared to the parent glucoamylase. In some aspects, the altered thermostability may be increased thermostability as compared to the parent glucoamylase. In some embodiments, the altered property is altered specific activity compared to the parent glucoamylase. In some embodiments, the altered specific activity may be increased specific activity compared to the parent glucoamylase. In some embodiments, the altered property is increased thermostability at lower temperatures as compared to the parent glucoamylase. In some embodiments, the altered property is both increased specific activity and increased thermostability as compared to the parent glucoamylase.

Some variants with multiple substitutions, i.e., combinatorial variants, may include the substitutions at positions:

I43Q/D44C/L417V/E503A/Q511H/A539R;

I43Q/L417V/E503A/Q511H/A539R;

I43Q/D44C/N61I/L417V/E503A/Q511H/A539R;

I43Q/N61I/L417V/E503A/Q511H/A539R;

I43R/L417V/E503A/Q511H/A539R;

I43R/N61I/L417V/E503A/Q511H/A539R;

I43R/L417R/E503A/A539R;

I43R/N61I/L417R/E503A/Q511H/A539R;

G73F/T430A/Q511H;

I43R/G73F/T430A;

G73F/T430A/E503V/Q511H;

D44C/G73F/N563K;

D44C/G73F/E503V/Q511H;

D44C/G73F/N563K;

D44C/G73F/L417R/N563K;

D44C/G73F/N563K;

I43R/T430A;

I43Q/T430A; I43Q/T430A/Q511H;

D44C/L417R/N563K;

L417V/T430A/A431L/Q511H/A535R/A539R/N5631;

L417V/T430A/A431Q/Q511H/A535R/A539R/N5631;

L417V/T430A/Q511H/A535R/N5631;

L417V/T430A/Q511H/A539R/N5631;

G294C/L417R/A431L;

G294C/L417V/A431Q;

G294C/L417V/A431L/Q511H;

G294C/L417R/A431Q/Q511H;

L417R/A431L/Q511H;

L417V/A431Q/Q511H;

I43Q/T430A/Q511H/N61I;

I43Q/T430A/Q511H/L417V;

I43Q/T430A/Q511H/A431L;

I43Q/T430A/Q511H/E503A;

I43Q/T430A/Q511H/A539R;

I43Q/T430A/Q511H/N61I/A539R;

I43Q/T430A/Q511H/L417V/A539R;

I43Q/T430A/Q511H/A431L/A539R;

I43Q/T430A/Q511H/A431L/E503A;

I43Q/T430A/Q511H/N61I/A539R/A431L;

I43Q/T430A/Q511H/L417V/A539R/A431L;

I43Q/Q511H/N61I;

I43Q/Q511H/L417V;

I43Q/Q511H/A431L;

I43Q/Q511H/A539R;

I43Q/Q511H/A539R/N61I;

I43Q/Q511H/A539R/E503A;

I43Q/Q511H/A539R/T430M;

I43Q/Q511H/A539R/T430M/N61I;

I43Q/Q511H/A539R/T430M/N61I/L417V;

I43R/T430A/E503V/A535R/N563K;

D44R/E503A/Q511H/N5631;

E503A/N563I;

I43R/T430A/E503A/Q511H/N563K;

D44R/T430A/Q511H/A535R;

L417V/A431L/A539R;

L417V/A431L/A539R/I43Q;

L417V/A431L/A539R/N61I;

L417V/A431L/A539R/A535R;

L417V/A431L/A539R/I43Q/N61I;

L417V/A431L/A539R/N61I/A535R;

L417V/A431L/A539R/A535R/I43Q;

L417V/A431L/A539R/I43Q/N61I/A535R;

L417V/A431L/A539R/I43Q/N61I/A535R/T430A;

L417V/T430A/A431L/Q511H/A535R/A539R/N5631/I43Q;

L417V/T430A/A431L/Q511H/A535R/A539R/N5631/N61I;

L417V/T430A/A431L/Q511H/A535R/A539R/N5631/I43Q/N61I;

L417V/A431L/A539R/I43R;

L417V/A431L/A539R/I43R/N61I;

L417V/A431L/A539R/I43R/N61I/A535R/T430A;

L417R/A431L/A539R;

L417G/A431L/A539R;

G73F/E503V/N563K/L417R/A539R;

G73F/E503V/N563K/I43R/L417R/A539R; and

G73F/E503V/N563K/I43R/Q511H

of SEQ ID NO: 2, or equivalent positions in parent glucoamylases and particularly Trichoderma glucoamylase homologues.

A number of parent glucoamylases have been aligned with the amino acid sequence of TrGA. FIG. 5 includes the catalytic domain of the following parent glucoamylases Aspergillus awamori (AaGA) (SEQ ID NO: 5); Aspergillus niger (AnGA) (SEQ ID NO: 6); Aspergillus orzyae (AoGA) (SEQ ID NO: 7); Humicola grisea (HgGA) (SEQ ID NO: 8); and Hypocrea vinosa (HvGA) (SEQ ID NO: 9). The % identity of the catalytic domains is represented in Table 1 below.

TABLE 1
Sequence homology between various fungal glucoamylases

| | AaGA | AnGA | AoGA | HgGA | HvGA | TrGA |
|---|---|---|---|---|---|---|
| AaGA | 100 | 95 | 58 | 53 | 57 | 56 |
| AnGA | | 100 | 59 | 53 | 57 | 56 |
| AoGA | | | 100 | 55 | 56 | 56 |
| HgGA | | | | 100 | 61 | 63 |
| HvGA | | | | | 100 | 91 |
| TrGA | | | | | | 100 |

In some embodiments, for example, the variant glucoamylase will be derived from a parent glucoamylase that is an Aspergillus glucoamylase, a Humicola glucoamylase, or a Hypocrea glucoamylase, and the variant will include at least one substitution in a position equivalent to a position set forth in SEQ ID NO: 2, and particularly in a position corresponding to: T10, L14, N15, P23, T42, I43, D44, P45, D46, F59, K60, N61, T67, E68, A72, G73, S97, L98, A99, S102, K108, E110, L113, K114, R122, Q124, R125, I133, K140, N144, N145, Y147, S152, N153, N164, F175, N182, A204, T205, S214, V216, Q219, W228, V229, S230, S231, D236, I239, N240, T241, N242, G244, N263, L264, G265, A268, G269, D276, V284, S291, G294, P300, A301, A303, Y310, A311, D313, Y316, V338, T342, S344, T346, A349, V359, G361, A364, T375, N379, S382, S390, E391, A393, K394, R408, S410, S415, L417, H418, T430, A431, R433, 1436, A442, N443, S444, T448, S451, T493, P494, T495, H₅₀₂, E503, Q508, Q511, N518, A519, A520, T527, V531, A535, V536, N537, A539, N563, or N577.

In some embodiments, the glucoamylase variant may differ from the parent glucoamylase only at the specified positions.

For example, the present invention provides glucoamylase variant comprising one of the following sets of substitutions, at positions of SEQ ID NO: 2 or equivalent positions in a parent glucoamylase:

L417V/A431L/A539R;

I43Q/L417V/A431L/A539R;

L417V/A431L/A535R/A539R

I43R/L417V/A431L/A539R;

L417R/A431L/A539R; or

L417G/A431L/A539R;

wherein the glucoamylase variant does not have any further substitutions relative to the parent glucoamylase, and wherein the parent glucoamylase has a catalytic domain that has at least 80% sequence identity with SEQ ID NO: 1, 2, 3, 5, 6, 7, 8, or 9. Thus the parent glucoamylase may be any of those described above.

The parent glucoamylase may comprise a starch binding domain that has at least 95% sequence identity with SEQ ID NO: 1, 2, 11, 385, 386, 387, 388, 389, or 390. The parent glucoamylase may have at least 80% sequence identity with SEQ ID NO: 1 or 2; for example it may comprise SEQ ID NO: 1 or 2. Optionally the parent glucoamylase may consist of SEQ ID NO: 1 or 2.

It will be apparent that the invention further extends to a method of preparing a glucoamylase variant as described herein, the method comprising providing a parent glucoamylase as described, and modifying said parent glucoamylase in order to provide said glucoamylase variant. The method may comprise the steps of providing a parent polynucleotide encoding said parent glucoamylase and modifying said parent polynucleotide to provide a variant polynucleotide encoding said glucoamylase variant. Such polynucleotides are described in more detail below. The methods of the invention may, for example, be used to generate a DNA construct or vector comprising a polynucleotide encoding a glucoamylase variant, also as described in more detail below.

5. Characterization of Variant Glucoamylases

The present disclosure also provides glucoamylase variants having at least one altered property (e.g., improved property) as compared to a parent glucoamylase and particularly to the TrGA. In some embodiments, at least one altered property (e.g., improved property) is selected from the group consisting of acid stability, thermal stability and specific activity. Typically, the altered property is increased acid stability, increased thermal stability and/or increased specific activity. The increased thermal stability typically is at higher temperatures. In one embodiment, the increased pH stability is at high pH. In a further embodiment, the increased pH stability is at low pH.

The glucoamylase variants of the disclosure may also provide higher rates of starch hydrolysis at low substrate concentrations as compared to the parent glucoamylase. The variant may have a higher Vₘₐₓ or lower Kₘ than a parent glucoamylase when tested under the same conditions. For example the variant glucoamylase may have a higher Vₘₐₓ at a temperature range of about 25° C. to about 70° C. (e.g., about 25° C. to about 35° C.; about 30° C. to about 35° C.; about 40° C. to about 50° C.; at about 50° C. to about 55° C., or about 55° C. to about 62° C.). The Michaelis-Menten constant, Kₘ and Vₘₐₓ values can be easily determined using standard known procedures.

5.1. Variant Glucoamylases with Altered Thermostability

In some aspects, the disclosure relates to a variant glucoamylase having altered thermal stability as compared to a parent (wild-type). Altered thermostability can be at increased temperatures or at decreased temperatures. Thermostability is measured as the % residual activity after incubation for 1 hour at 64° C. in NaAc buffer pH 4.5. Under these conditions, TrGA has a residual activity of between about 15% and 44% due to day-to-day variation as compared to the initial activity before incubation. Thus, in some embodiments, variants with increased thermostability have a residual activity that is between at least about 1% and at least about 50% more than that of the parent (after incubation for 1 hour at 64° C. in NaAc buffer pH 4.5), including about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, and about 50% as compared to the initial activity before incubation. For example, when the parent residual activity is 15%, a variant with increased thermal stability may have a residual activity of between about 16% and about 75%. In some embodiments, the glucoamylase variant will have improved thermostability such as retaining at least about 50%, about 60%, about 70%, about 75%, about 80%, about 85%, about 90%, about 92%, about 95%, about 96%, about 97%, about 98%, or about 99% enzymatic activity after exposure to altered temperatures over a given time period, for example, at least about 60 minutes, about 120 minutes, about 180 minutes, about 240 minutes, or about 300 minutes. In some embodiments, the variant has increased thermal stability compared to the parent glucoamylase at selected temperatures in the range of about 40° C. to about 80° C., also in the range of about 50° C. to about 75° C., and in the range of about 60° C. to about 70° C., and at a pH range of about 4.0 to about 6.0. In some embodiments, the thermostability is determined as described in the Assays and Methods. That method may be adapted as appropriate to measure thermostability at other temperatures. Alternatively the thermostability may be determined at 64° C. as described there. In some embodiments, the variant has increased thermal stability at lower temperature compared to the parent glucoamylase at selected temperature in the range of about 20° C. to about 50° C., including about 35° C. to about 45° C. and about 30° C. to about 40° C.

In some embodiments, variants having an improvement in thermostability include one or more deletions, substitutions or insertions and particularly substitutions in the following positions in the amino acid sequence set forth in SEQ ID NO: 2: 10, 42, 43, 44, 59, 61, 68, 72, 73, 97, 98, 99, 102, 114, 133, 140, 144, 152, 153, 182, 204, 205, 214, 216, 228, 229, 230, 231, 236, 241, 242, 263, 264, 265, 268, 269, 276, 284, 291, 294 300, 301, 303, 311, 338, 342, 344, 346, 349, 359, 361, 364, 375, 379, 382, 390, 391, 393, 394, 410, 417, 430, 431, 433, 436, 442, 444, 448, 451, 493, 495, 503, 508, 511, 518, 519, 520, 527, 531, 535, 536, 537, 539, 563, or 577, or an equivalent position in a parent glucoamylase. In some embodiments, the parent glucoamylase will be a Trichoderma glucoamylase homologue and in further embodiments, the parent glucoamylase will have at least about 50%, about 60%, about 70%, about 80%, about 90%, about 95%, or about 98% sequence identity to SEQ ID NO: 2. In some embodiments, the parent glucoamylase will also have structural identity to SEQ ID NO: 2. In some embodiments, the variant having increased thermostability has a substitution in at least one of the positions: T105, T42V, I43Q, I43R, D44C, D44R, E68C, E68M, G73F, G73W, K114M, K114Q, I133V, N153A, N153E, N153M, N153S, N153V, W228V, V229I, V229L, S230Q, S231V, D236R, L264D, L264K, A268D, S291A, S291F, S291H, S291M, S291T, G294C, A301P, A301R, V338I, V338N, V338Q, S344M, S344P, S344Q, S344R, S344V, G361D, G361E, G361F, G361I, G361L, G361M, G361P, G361S, G361W, G361Y, A364D, A364E, A364F, A364G, A364K, A364L, A364M, A364R, A364S, A364T, A364V, A364W, T375N, L417K, L417R, R433c, R433E, R433G, R433L, R433N, R433S, R433V, I436H, T495K, T495S, E503A, E503C, E503V, Q508R, Q511H, A519K, A519R, A519Y, V531L, A535K, A535N, A535P, A535R, A539E, A539R, A5395, N563C, N563E, N563I, N563K, N563L, N563Q, N563T, N563V, N577K, N577P, or N577R of SEQ ID NO: 2.

5.2. Variant Glucoamylases with Altered Specific Activity

As used herein, specific activity is the activity of the glucoamylase per mg of protein. Activity was determined using the ethanol assay. The screening identified variants having a Performance Index (PI)>1.0 compared to the parent TrGA PI. The PI is calculated from the specific activities (activity/mg enzyme) of the wild-type (WT) and the variant enzymes. It is the quotient “Variant-specific activity/WT-specific activity” and can be a measure of the increase in specific activity of the variant. A PI of about 2 should be about 2 fold better than WT. In some aspects, the disclosure relates to a variant glucoamylase having altered specific activity as compared to a parent or wild-type glucoamylase. In some embodiments, the altered specific activity is increased specific activity. Increased specific activity can be defined as an increased performance index of greater than or equal to about 1, including greater than or equal to about 1.1, about 1.2, about 1.3, about 1.4, about 1.5, about 1.6, about 1.7, about 1.8, about 1.9, and about 2. In some embodiments, the increased specific activity is from about 1.0 to about 5.0, including about 1.1, about 1.2, about 1.3, about 1.4, about 1.5, about 1.6, about 1.7, about 1.8, about 1.9, about 2.0, about 2.1, about 2.2., about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, and about 4.9. In some embodiments, the variant has an at least about 1.0 fold higher specific activity than the parent glucoamylase, including at least about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.2 fold, about 2.5 fold, about 2.7 fold, about 2.9 fold, about 3.0 fold, about 4.0 fold, and about 5.0 fold.

In some embodiments, variants having an improvement in specific activity include one or more deletions, substitutions or insertions in the following positions in the amino acid sequence set forth in SEQ ID NO: 2: 10, 14, 15, 23, 59, 60, 61, 65, 67, 68, 72, 73, 97, 98, 99, 102, 110, 113, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 417, 418, 430, 431, 433, 442, 444, 448, 451, 493, 494, 495, 502, 503, 508, 511, 518, 519, 520, 531, 535, 536, 539, or 563, or an equivalent position in a parent glucoamylase. In some embodiments, the parent glucoamylase will comprise a sequence having at least about 50%, about 60%, about 70%, about 80%, about 90%, or about 95% sequence identity to the sequence of SEQ ID NO: 2. In some embodiments, the parent glucoamylase will also have structural identity to SEQ ID NO: 2. In some embodiments, variants of the disclosure having improved specific activity include a substitution in the following positions in the amino acid sequence set forth in SEQ ID NO: 2: I43Q, I43R, D44C, D44R, N061I, T067M, A072Y, S097N, S102A, S102M, S102R, I133T, N145I, N153D, T205Q, Q219S, W228A, W228F, W228H, W228M, S230C, S230F, S230G, S230L, S230N, S230Q, S230R, S231L, I239V, I239Y, N263P, A268C, A268G, A268K, S291A, G294C, T342V, K394S, L417R, L417V, T430K, A431I, A431L, A431Q, R433Y, T451K, T495M, A519I, A520C, A520L, A520P, A535R, V536M, A539R, N563K, or N563I, or an equivalent position in a parent glucoamylase. In some embodiments, the specific activity of the parent as compared to the variant is determined as described in the Assays and Methods.

5.3. Variant Glucoamylases with Both Altered Thermostability and Altered Specific Activity

In some aspects, the disclosure relates to a variant glucoamylase having both altered thermostability and altered specific activity as compared to a parent (e.g., wild-type). In some embodiments, the altered specific activity is an increased specific activity. In some embodiments, the altered thermostability is an increased thermostability at high temperatures (e.g., at temperatures above 80° C.) as compared to the parent glucoamylase.

In some embodiments, variants with an increased thermostability and increased specific activity include one or more deletions, substitutions or insertions and substitutions in the following positions in the amino acid sequence set forth in SEQ ID NO: 2: 10, 15, 43, 44, 59, 61, 68, 72, 73, 97, 99, 102, 140, 153, 182, 204, 205, 214, 228, 229, 230, 231, 236, 241, 242, 264, 265, 268, 276, 284, 291, 294, 300, 301, 303, 311, 338, 344, 346, 349, 359, 361, 364, 375, 379, 382, 391, 393, 394, 410, 430, 433, 444, 448, 451, 495, 503, 511, 520, 531, 535, 536, 539, or 563, or an equivalent position in a parent glucoamylase. In some embodiments, the parent glucoamylase will be a Trichoderma glucoamylase homologue and in further embodiments, the parent glucoamylase will have at least about 50%, about 60%, about 70%, about 80%, about 90%, about 95%, or about 98% sequence identity to SEQ ID NO:2. In some embodiments, the parent glucoamylase will also have structural identity to SEQ ID NO: 2. In some embodiments, the variant having increased thermostability and specific activity has a substitution in at least one of the positions: I43Q/R, D44C/R, W228F/H/M, S230C/F/G/N/Q/R, S231L, A268C/D/G/K, S291A, G294C, R433Y, S451K, E503C, Q511H, A520C/L/P, or A535N/P/R of SEQ ID NO: 2.

6. Polynucleotides Encoding Glucoamylases

The present disclosure also relates to isolated polynucleotides encoding the variant glucoamylase. The polynucleotides may be prepared by established techniques known in the art. The polynucleotides may be prepared synthetically, such as by an automatic DNA synthesizer. The DNA sequence may be of mixed genomic (or cDNA) and synthetic origin prepared by ligating fragments together. The polynucleotides may also be prepared by polymerase chain reaction (PCR) using specific primers. In general, reference is made to Minshull J. et al., Methods 32(4):416-427 (2004). DNA may also be synthesized by a number of commercial companies such as Geneart AG, Regensburg, Germany.

The present disclosure also provides isolated polynucleotides comprising a nucleotide sequence (i) having at least about 50% identity to SEQ ID NO: 4, including at least about 60%, about 70%, about 80%, about 90%, about 95%, and about 99%, or (ii) being capable of hybridizing to a probe derived from the nucleotide sequence set forth in SEQ ID NO: 4, under conditions of intermediate to high stringency, or (iii) being complementary to a nucleotide sequence having at least 90% sequence identity to the sequence set forth in SEQ ID NO: 4. Probes useful according to the disclosure may include at least about 50, about 100, about 150, about 200, about 250, about 300 or more contiguous nucleotides of SEQ ID NO: 4. In some embodiments, the encoded polypeptide also has structural identity to SEQ ID NO: 2.

The present disclosure further provides isolated polynucleotides that encode variant glucoamylases that comprise an amino acid sequence comprising at least about 50%, about 60%, about 70%, about 80%, about 90%, about 93%, about 95%, about 97%, about 98%, or about 99% amino acid sequence identity to SEQ ID NO: 2. Additionally, the present disclosure provides expression vectors comprising any of the polynucleotides provided above. The present disclosure also provides fragments (i.e., portions) of the DNA encoding the variant glucoamylases provided herein. These fragments find use in obtaining partial length DNA fragments capable of being used to isolate or identify polynucleotides encoding mature glucoamylase enzymes described herein from filamentous fungal cells (e.g., Trichoderma, Aspergillus, Fusarium, Penicillium, and Humicola), or a segment thereof having glucoamylase activity. In some embodiments, fragments of the DNA may comprise at least about 50, about 100, about 150, about 200, about 250, about 300 or more contiguous nucleotides. In some embodiments, portions of the DNA provided in SEQ ID NO: 4 may be used to obtain parent glucoamylases and particularly Trichoderma glucoamylase homologues from other species, such as filamentous fungi that encode a glucoamylase.

7. Production of Glucoamylases

7.1. DNA Constructs and Vectors

According to one embodiment of the disclosure, a DNA construct comprising a polynucleotide as described above encoding a variant glucoamylase encompassed by the disclosure and operably linked to a promoter sequence is assembled to transfer into a host cell.

The DNA construct may be introduced into a host cell using a vector. The vector may be any vector that when introduced into a host cell is stably introduced. In some embodiments, the vector is integrated into the host cell genome and is replicated. Vectors include cloning vectors, expression vectors, shuttle vectors, plasmids, phage particles, cassettes and the like. In some embodiments, the vector is an expression vector that comprises regulatory sequences operably linked to the glucoamylase coding sequence.

Examples of suitable expression and/or integration vectors are provided in Sambrook et al. (1989) supra, and Ausubel (1987) supra, and van den Hondel et al. (1991) in Bennett and Lasure (Eds.) MORE GENE MANIPULATIONS IN FUNGI, Academic Press pp. 396-428 and U.S. Pat. No. 5,874,276. Reference is also made to the Fungal Genetics Stock Center Catalogue of Strains (FGSC, http://www.fgsc.net) for a list of vectors. Particularly useful vectors include vectors obtained from for example Invitrogen and Promega.

Suitable plasmids for use in bacterial cells include pBR322 and pUC19 permitting replication in E. coli and pE194 for example permitting replication in Bacillus. Other specific vectors suitable for use in E. coli host cells include vectors such as pFB6, pBR322, pUC18, pUC100, pDONR™201, 10 pDONR™221, pENTR™, pGEM®3Z and pGEM®4Z.

Specific vectors suitable for use in fungal cells include pRAX, a general purpose expression vector useful in Aspergillus, pRAX with a glaA promoter, and in Hypocrea/Trichoderma includes pTrex3g with a cbh1 promoter.

In some embodiments, the promoter that shows transcriptional activity in a bacterial or a fungal host cell may be derived from genes encoding proteins either homologous or heterologous to the host cell. The promoter may be a mutant, a truncated and/or a hybrid promoter. The above-mentioned promoters are known in the art. Examples of suitable promoters useful in fungal cells and particularly filamentous fungal cells such as Trichoderma or Aspergillus cells include such exemplary promoters as the T. reesei promoters cbh1, cbh2, egl1, egl2, eg5, xln1 and xln2. Other examples of useful promoters include promoters from A. awamori and A. niger glucoamylase genes (glaA) (see Nunberg et al., Mol. Cell. Biol. 4: 2306-2315 (1984) and Boel et al., EMBO J. 3:1581-1585 (1984)), A. oryzae TAKA amylase promoter, the TPI (triose phosphate isomerase) promoter from S. cerevisiae, the promoter from Aspergillus nidulans acetamidase genes and Rhizomucor miehei lipase genes. Examples of suitable promoters useful in bacterial cells include those obtained from the E. coli lac operon; Bacillus lichenifonnis alpha-amylase gene (amyL), B. stearothermophilus amylase gene (amyS); Bacillus subtilis xylA and xylB genes, the beta-lactamase gene, and the tac promoter. In some embodiments, the promoter is one that is native to the host cell. For example, when T. reesei is the host, the promoter is a native T. reesei promoter. In other embodiments, the promoter is one that is heterologous to the fungal host cell. In some embodiments, the promoter will be the promoter of a parent glucoamylase (e.g., the TrGA promoter).

In some embodiments, the DNA construct includes nucleic acids coding for a signal sequence, that is, an amino acid sequence linked to the amino terminus of the polypeptide that directs the encoded polypeptide into the cell's secretory pathway. The 5′ end of the coding sequence of the nucleic acid sequence may naturally include a signal peptide coding region that is naturally linked in translation reading frame with the segment of the glucoamylase coding sequence that encodes the secreted glucoamylase or the 5′ end of the coding sequence of the nucleic acid sequence may include a signal peptide that is foreign to the coding sequence. In some embodiments, the DNA construct includes a signal sequence that is naturally associated with a parent glucoamylase gene from which a variant glucoamylase has been obtained. In some embodiments, the signal sequence will be the sequence depicted in SEQ ID NO: 1 or a sequence having at least about 90%, about 94, or about 98% sequence identity thereto. Effective signal sequences may include the signal sequences obtained from other filamentous fungal enzymes, such as from Trichoderma (T. reesei glucoamylase, cellobiohydrolase I, cellobiohydrolase II, endoglucanase I, endoglucanase II, endoglucanase II, or a secreted proteinase, such as an aspartic proteinase), Humicola (H. insolens cellobiohydrolase or endoglucanase, or H. grisea glucoamylase), or Aspergillus (A. niger glucoamylase and A. oryzae TAKA amylase).

In additional embodiments, a DNA construct or vector comprising a signal sequence and a promoter sequence to be introduced into a host cell are derived from the same source. In some embodiments, the native glucoamylase signal sequence of a Trichoderma glucoamylase homologue, such as a signal sequence from a Hypocrea strain may be used.

In some embodiments, the expression vector also includes a termination sequence. Any termination sequence functional in the host cell may be used in the present disclosure. In some embodiments, the termination sequence and the promoter sequence are derived from the same source. In another embodiment, the termination sequence is homologous to the host cell. Useful termination sequences include termination sequences obtained from the genes of Trichoderma reesei cbl1; A. niger or A. awamori glucoamylase (Nunberg et al. (1984) supra, and Boel et al., (1984) supra), Aspergillus nidulans anthranilate synthase, Aspergillus oryzae TAKA amylase, or A. nidulans trpC (Punt et al., Gene 56:117-124 (1987)).

In some embodiments, an expression vector includes a selectable marker. Examples of selectable markers include ones that confer antimicrobial resistance (e.g., hygromycin and phleomycin). Nutritional selective markers also find use in the present disclosure including those markers known in the art as amdS (acetamidase), argB (ornithine carbamoyltransferase) and pyrG (orotidine-5′ phosphate decarboxylase). Markers useful in vector systems for transformation of Trichoderma are known in the art (see, e.g., Finkelstein, Chapter 6 in BIOTECHNOLOGY OF FILAMENTOUS FUNGI, Finkelstein et al. (1992) Eds. Butterworth-Heinemann, Boston, Mass.; Kinghorn et al. (1992) APPLIED MOLECULAR GENETICS OF FILAMENTOUS FUNGI, Blackie Academic and Professional, Chapman and Hall, London; Berges and Barreau, Curr. Genet. 19:359-365 (1991); and van Hartingsveldt et al., Mol. Gen. Genet. 206:71-75 (1987)). In some embodiments, the selective marker is the amdS gene, which encodes the enzyme acetamidase, allowing transformed cells to grow on acetamide as a nitrogen source. The use of A. nidulans amdS gene as a selective marker is described in Kelley et al., EMBO J. 4:475-479 (1985) and Penttila et al., Gene 61:155-164 (1987).

Methods used to ligate the DNA construct comprising a nucleic acid sequence encoding a variant glucoamylase, a promoter, a termination and other sequences and to insert them into a suitable vector are well known in the art. Linking is generally accomplished by ligation at convenient restriction sites. If such sites do not exist, synthetic oligonucleotide linkers are used in accordance with conventional practice (see Sambrook et al. (1989) supra, and Bennett and Lasure, MORE GENE MANIPULATIONS IN FUNGI, Academic Press, San Diego (1991) pp 70-76.). Additionally, vectors can be constructed using known recombination techniques (e.g., Invitrogen Life Technologies, Gateway Technology).

7.2. Host Cells and Transformation of Host Cells

The present disclosure also relates to host cells comprising a polynucleotide encoding a variant glucoamylase of the disclosure. In some embodiments, the host cells are chosen from bacterial, fungal, plant and yeast cells. The term host cell includes both the cells, progeny of the cells and protoplasts created from the cells that are used to produce a variant glucoamylase according to the disclosure.

In some embodiments, the host cells are fungal cells and optionally filamentous fungal host cells. The term “filamentous fungi” refers to all filamentous forms of the subdivision Eumycotina (see, Alexopoulos, C. J. (1962), INTRODUCTORY MYCOLOGY, Wiley, New York). These fungi are characterized by a vegetative mycelium with a cell wall composed of chitin, cellulose, and other complex polysaccharides. The filamentous fungi of the present disclosure are morphologically, physiologically, and genetically distinct from yeasts. Vegetative growth by filamentous fungi is by hyphal elongation and carbon catabolism is obligatory aerobic. In the present disclosure, the filamentous fungal parent cell may be a cell of a species of, but not limited to, Trichoderma (e.g., Trichoderma reesei, the asexual morph of Hypocrea jecorina, previously classified as T. longibrachiatum, Trichoderma viride, Trichoderma koningii, Trichoderma harzianum) (Sheir-Neirs et al., Appl. Microbiol. Biotechnol. 20:46-53 (1984); ATCC No. 56765 and ATCC No. 26921), Penicilliurn sp., Humicola sp. (e.g., H. insolens, H. lanuginosa and H. grisea), Chrysosporium sp. (e.g., C. lucknowense), Gliocladium sp., Aspergillus sp. (e.g., A. oryzae, A. niger, A sojae, A. japonicus, A. nidulans, and A. awamori) (Ward et al., Appl. Microbiol. Biotechnol. 39:738-743 (1993) and Goedegebuur et al., Curr. Genet. 41:89-98 (2002)), Fusarium sp., (e.g., F. roseum, F. graminum, F. cerealis, F. oxysporum, and F. venenatum), Neurospora sp., (N. crassa), Hypocrea sp., Mucor sp. (M. miehei), Rhizopus sp., and Emericella sp. (see also, Innis et al., Science 228:21-26 (1985)). The term “Trichoderma” or “Trichoderma sp.” or “Trichoderma spp.” refer to any fungal genus previously or currently classified as Trichoderma.

In some embodiments, the host cells will be gram-positive bacterial cells. Non-limiting examples include strains of Streptomyces (e.g., S. lividans, S. coelicolor, and S. griseus) and Bacillus. As used herein, “the genus Bacillus” includes all species within the genus “Bacillus,” as known to those of skill in the art, including but not limited to B. subtilis, B. lichenifonnis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus tearothermophilus.”

In some embodiments, the host cell is a gram-negative bacterial strain, such as E. coli or Pseudomonas sp. In other embodiments, the host cells may be yeast cells such as Saccharomyces sp., Schizosaccharomyces sp., Pichia sp., or Candida sp. In other embodiments, the host cell will be a genetically engineered host cell wherein native genes have been inactivated, for example by deletion in bacterial or fungal cells. Where it is desired to obtain a fungal host cell having one or more inactivated genes known methods may be used (e.g., methods disclosed in U.S. Pat. No. 5,246,853, U.S. Pat. No. 5,475,101, and WO 92/06209). Gene inactivation may be accomplished by complete or partial deletion, by insertional inactivation or by any other means that renders a gene nonfunctional for its intended purpose (such that the gene is prevented from expression of a functional protein). In some embodiments, when the host cell is a Trichoderma cell and particularly a T. reesei host cell, the cbh1, cbh2, egl1 and egl2 genes will be inactivated and/or deleted. Exemplary Trichoderma reesei host cells having quad-deleted proteins are set forth and described in U.S. Pat. No. 5,847,276 and WO 05/001036. In other embodiments, the host cell is a protease deficient or protease minus strain.

Introduction of a DNA construct or vector into a host cell includes techniques such as transformation; electroporation; nuclear microinjection; transduction; transfection, (e.g., lipofection-mediated and DEAE-Dextrin mediated transfection); incubation with calcium phosphate DNA precipitate; high velocity bombardment with DNA-coated microprojectiles; and protoplast fusion. General transformation techniques are known in the art (see, e.g., Ausubel et al. (1987) supra, chapter 9; and Sambrook et al. (1989) supra, and Campbell et al., Curr. Genet. 16:53-56 (1989)).

Transformation methods for Bacillus are disclosed in numerous references including Anagnostopoulos C. and J. Spizizen, J. Bacteriol. 81:741-746 (1961) and WO 02/14490.

Transformation methods for Aspergillus are described in Yelton et al., Proc. Natl. Acad. Sci. USA 81:1470-1474 (1984); Berka et al., (1991) in APPLICATIONS OF ENZYME BIOTECHNOLOGY, Eds. Kelly and Baldwin, Plenum Press (NY); Cao et al., Protein Sci. 9:991-1001 (2000); Campbell et al., Curr. Genet. 16:53-56 (1989), and EP 238 023. The expression of heterologous protein in Trichoderma is described in U.S. Pat. No. 6,022,725; U.S. Pat. No. 6,268,328; Harkki et al. Enzyme Microb. Technol. 13:227-233 (1991); Harkki et al., BioTechnol. 7:596-603 (1989); EP 244,234; EP 215,594; and Nevalainen et al., “The Molecular Biology of Trichoderma and its Application to the Expression of Both Homologous and Heterologous Genes”, in MOLECULAR INDUSTRIAL MYCOLOGY, Eds. Leong and Berka, Marcel Dekker Inc., NY (1992) pp. 129-148). Reference is also made to WO96/00787 and Bajar et al., Proc. Natl. Acad. Sci. USA 88:8202-8212 (1991) for transformation of Fusarium strains.

In one specific embodiment, the preparation of Trichoderma sp. for transformation involves the preparation of protoplasts from fungal mycelia (see, Campbell et al., Curr. Genet. 16:53-56 (1989); Pentilla et al., Gene 61:155-164 (1987)). Agrobacterium tumefaciens-mediated transformation of filamentous fungi is known (see de Groot et al., Nat. Biotechnol. 16:839-842 (1998)). Reference is also made to U.S. Pat. No. 6,022,725 and U.S. Pat. No. 6,268,328 for transformation procedures used with filamentous fungal hosts.

In some embodiments, genetically stable transformants are constructed with vector systems whereby the nucleic acid encoding the variant glucoamylase is stably integrated into a host strain chromosome. Transformants are then purified by known techniques.

In some further embodiments, the host cells are plant cells, such as cells from a monocot plant (e.g., corn, wheat, and sorghum) or cells from a dicot plant (e.g., soybean). Methods for making DNA constructs useful in transformation of plants and methods for plant transformation are known. Some of these methods include Agrobacterium tumefaciens mediated gene transfer; microprojectile bombardment, PEG mediated transformation of protoplasts, electroporation and the like. Reference is made to U.S. Pat. No. 6,803,499, U.S. Pat. No. 6,777,589; Fromm et al., BioTechnol. 8:833-839 (1990); Potrykus et al., Mol. Gen. Genet. 199:169-177 (1985).

7.3. Production of Glucoamylases

The present disclosure further relates to methods of producing the variant glucoamylases, which comprises transforming a host cell with an expression vector comprising a polynucleotide encoding a variant glucoamylase according to the disclosure, culturing the host cell under conditions suitable for expression and production of the variant glucoamylase and optionally recovering the variant glucoamylase.

In the expression and production methods of the present disclosure the host cells are cultured under suitable conditions in shake flask cultivation, small scale or large scale fermentations (including continuous, batch and fed batch fermentations) in laboratory or industrial fermentors, with suitable medium containing physiological salts and nutrients (see, e.g., Pourquie, J. et al., BIOCHEMISTRY AND GENETICS OF CELLULOSE DEGRADATION, eds. Aubert, J. P. et al., Academic Press, pp. 71-86, 1988 and Ilmen, M. et al., Appl. Environ. Microbiol. 63:1298-1306 (1997)). Common commercially prepared media (e.g., Yeast Malt Extract (YM) broth, Luria Bertani (LB) broth and Sabouraud Dextrose (SD) broth) find use in the present disclosure. Culture conditions for bacterial and filamentous fungal cells are known in the art and may be found in the scientific literature and/or from the source of the fungi such as the American Type Culture Collection and Fungal Genetics Stock Center. In cases where a glucoamylase coding sequence is under the control of an inducible promoter, the inducing agent (e.g., a sugar, metal salt or antimicrobial), is added to the medium at a concentration effective to induce glucoamylase expression.

In some embodiments, the present disclosure relates to methods of producing the variant glucoamylase in a plant host comprising transforming a plant cell with a vector comprising a polynucleotide encoding a glucoamylase variant according to the disclosure and growing the plant cell under conditions suitable for the expression and production of the variant.

In some embodiments, assays are carried out to evaluate the expression of a variant glucoamylase by a cell line that has been transformed with a polynucleotide encoding a variant glucoamylase encompassed by the disclosure. The assays can be carried out at the protein level, the RNA level and/or by use of functional bioassays particular to glucoamylase activity and/or production. Some of these assays include Northern blotting, dot blotting (DNA or RNA analysis), RT-PCR (reverse transcriptase polymerase chain reaction), in situ hybridization using an appropriately labeled probe (based on the nucleic acid coding sequence) and conventional Southern blotting and autoradiography.

In addition, the production and/or expression of a variant glucoamylase may be measured in a sample directly, for example, by assays directly measuring reducing sugars such as glucose in the culture medium and by assays for measuring glucoamylase activity, expression and/or production. In particular, glucoamylase activity may be assayed by the 3,5-dinitrosalicylic acid (DNS) method (see Goto et al., Biosci. Biotechnol. Biochem. 58:49-54 (1994)). In additional embodiments, protein expression, is evaluated by immunological methods, such as immunohistochemical staining of cells, tissue sections or immunoassay of tissue culture medium, (e.g., by Western blot or ELISA). Such immunoassays can be used to qualitatively and quantitatively evaluate expression of a glucoamylase. The details of such methods are known to those of skill in the art and many reagents for practicing such methods are commercially available.

The glucoamylases of the present disclosure may be recovered or purified from culture media by a variety of procedures known in the art including centrifugation, filtration, extraction, precipitation and the like.

8. Compositions and Uses

The variant glucoamylases of the disclosure may be used in enzyme compositions including but not limited to starch hydrolyzing and saccharifying compositions, cleaning and detergent compositions (e.g., laundry detergents, dish washing detergents, and hard surface cleaning compositions), alcohol fermentation compositions, and in animal feed compositions. Further, the variant glucoamylases may be used in, for example, brewing, healthcare, textile, environmental waste conversion processes, biopulp processing, and biomass conversion applications.

In some embodiments, an enzyme composition comprising a variant glucoamylase encompassed by the disclosure will be optionally used in combination with any one or combination of the following enzymes-alpha-amylases, proteases, pullulanases, isoamylases, cellulases, hemicellulases, xylanases, cyclodextrin glycotransferases, lipases, phytases, laccases, oxidases, esterases, cutinases, xylanases, granular starch hydrolyzing enzymes and other glucoamylases.

In some embodiments, the enzyme composition will include an alpha-amylase such as fungal alpha-amylases (e.g., Aspergillus sp.) or bacterial alpha-amylases (e.g., Bacillus sp. such as B. stearothermophilus, B. amyloliquefaciens and B. lichenifonnis) and variants and hybrids thereof. In some embodiments, the alpha-amylase is an acid stable alpha-amylase. In some embodiments, the alpha-amylase is Aspergillus kawachi alpha-amylase (AkAA), see U.S. Pat. No. 7,037,704. Commercially available alpha-amylases contemplated for use in the compositions of the disclosure are known and include GZYME G997, SPEZYME® FRED, SPEZYME® XTRA (Danisco US, Inc, Genencor Division), TERMAMYL® 120-L and SUPRA® (Novozymes, A/S).

In some embodiments, the enzyme composition will include an acid fungal protease. In a further embodiment, the acid fungal protease is derived from a Trichoderma sp and may be any one of the proteases disclosed in U.S. Pat. No. 7,563,607 (published as US 2006/0154353 Jul. 13, 2006), incorporated herein by reference. In a further embodiment, the enzyme composition will include a phytase from Buttiauxiella spp. (e.g., BP-17, see also variants disclosed in PCT patent publication WO 2006/043178).

In other embodiments, the variant glucoamylases of the disclosure may be combined with other glucoamylases. In some embodiments, the glucoamylases of the disclosure will be combined with one or more glucoamylases derived from strains of Aspergillus or variants thereof, such as A. oryzae, A. niger, A. kawachi, and A. awamori; glucoamylases derived from strains of Humicola or variants thereof, particularly H. grisea, such as the glucoamylase having at least about 90%, about 93%, about 95%, about 96%, about 97%, about 98%, or about 99% sequence identity to SEQ ID NO: 3 disclosed in WO 05/052148; glucoamylases derived from strains of Talaromyces or variants thereof, particularly T. emersonii; glucoamylases derived from strains of Athelia and particularly A. rolfsii; glucoamylases derived from strains of Penicillium, particularly P. chrysogenum.

In particular, the variant glucoamylases may be used for starch conversion processes, and particularly in the production of dextrose for fructose syrups, specialty sugars and in alcohol and other end-product (e.g., organic acid, ascorbic acid, and amino acids) production from fermentation of starch containing substrates (G.M.A. van Beynum et al., Eds. (1985) STARCH CONVERSION TECHNOLOGY, Marcel Dekker Inc. NY). Dextrins produced using variant glucoamylase compositions of the disclosure may result in glucose yields of at least 80%, at least 85%, at least 90% and at least 95%. Production of alcohol from the fermentation of starch substrates using glucoamylases encompassed by the disclosure may include the production of fuel alcohol or potable alcohol. In some embodiments, the production of alcohol will be greater when the variant glucoamylase is used under the same conditions as the parent glucoamylase. In some embodiments, the production of alcohol will be between about 0.5% and 2.5% better, including but not limited to about 0.6%, about 0.7%, about 0.8%, about 0.9%, about 1.0%, about 1.1%, about 1.2%, about 1.3%, about 1.4%, about 1.5%, about 1.6%. about 1.7%, about 1.8%, about 1.9%, about 2.0%, about 2.1%, about 2.2%, about 2.3%, and about 2.4% more alcohol than the parent glucoamylase.

In some embodiments, the variant glucoamylases of the disclosure will find use in the hydrolysis of starch from various plant-based substrates, which are used for alcohol production. In some embodiments, the plant-based substrates will include corn, wheat, barley, rye, milo, rice, sugar cane, potatoes and combinations thereof. In some embodiments, the plant-based substrate will be fractionated plant material, for example a cereal grain such as corn, which is fractionated into components such as fiber, germ, protein and starch (endosperm) (U.S. Pat. No. 6,254,914 and U.S. Pat. No. 6,899,910). Methods of alcohol fermentations are described in THE ALCOHOL TEXTBOOK, K. A. Jacques et al., Eds. 2003, Nottingham University Press, UK.

In certain embodiments, the alcohol will be ethanol. In particular, alcohol fermentation production processes are characterized as wet milling or dry milling processes. In some embodiments, the variant glucoamylase will be used in a wet milling fermentation process and in other embodiments the variant glucoamylase will find use in a dry milling process.

Dry grain milling involves a number of basic steps, which generally include: grinding, cooking, liquefaction, saccharification, fermentation and separation of liquid and solids to produce alcohol and other co-products. Plant material and particularly whole cereal grains, such as corn, wheat or rye are ground. In some cases, the grain may be first fractionated into component parts. The ground plant material may be milled to obtain a coarse or fine particle. The ground plant material is mixed with liquid (e.g., water and/or thin stillage) in a slurry tank. The slurry is subjected to high temperatures (e.g., about 90° C. to about 105° C. or higher) in a jet cooker along with liquefying enzymes (e.g., alpha-amylases) to solublize and hydrolyze the starch in the grain to dextrins. The mixture is cooled down and further treated with saccharifying enzymes, such as glucoamylases encompassed by the instant disclosure, to produce glucose. The mash containing glucose may then be fermented for approximately 24 to 120 hours in the presence of fermentation microorganisms, such as ethanol producing microorganism and particularly yeast (Saccharomyces spp). The solids in the mash are separated from the liquid phase and alcohol such as ethanol and useful co-products such as distillers' grains are obtained.

In some embodiments, the saccharification step and fermentation step are combined and the process is referred to as simultaneous saccharification and fermentation or simultaneous saccharification, yeast propagation and fermentation.

In other embodiments, the variant glucoamylase is used in a process for starch hydrolysis wherein the temperature of the process is between about 30° C. and about 75° C., in some embodiments, between about 40° C. and about 65° C. In some embodiments, the variant glucoamylase is used in a process for starch hydrolysis at a pH between about 3.0 and about 6.5. The fermentation processes in some embodiments include milling of a cereal grain or fractionated grain and combining the ground cereal grain with liquid to form a slurry that is then mixed in a single vessel with a variant glucoamylase according to the disclosure and optionally other enzymes such as, but not limited to, alpha-amylases, other glucoamylases, phytases, proteases, pullulanases, isoamylases or other enzymes having granular starch hydrolyzing activity and yeast to produce ethanol and other co-products (see e.g., U.S. Pat. No. 4,514,496, WO 04/081193, and WO 04/080923).

In some embodiments, the disclosure pertains to a method of saccharifying a liquid starch solution, which comprises an enzymatic saccharification step using a variant glucoamylase of the disclosure.

In other embodiments, the variant glucoamylase is used in a process for beer brewing. Brewing processes are well-known in the art, and generally involve the steps of malting, mashing, and fermentation. Mashing is the process of converting starch from the milled barley malt and solid adjuncts into fermentable and un-fermentable sugars to produce wort. Traditional mashing involves mixing milled barley malt and adjuncts with water at a set temperature and volume to continue the biochemical changes initiated during the malting process. The mashing process is conducted over a period of time at various temperatures in order to activate the endogenous enzymes responsible for the degradation of proteins and carbohydrates. After mashing, the wort is separated from the solids (spent grains). Following wort separation, the wort may be fermented with brewers yeast to produce a beer. The short-branched glucose oligomers formed during mashing may be further hydrolyzed by addition of exogenous enzymes like glucoamylases and/or alpha-amylases, beta-amylases and pullulanase, among others. The wort may be used as it is or it may be concentrated and/or dried. The concentrated and/or dried wort may be used as brewing extract, as malt extract flavoring, for non-alcoholic malt beverages, malt vinegar, breakfast cereals, for confectionary etc. The wort is fermented to produce an alcoholic beverage, typically a beer, e.g., ale, strong ale, bitter, stout, porter, lager, export beer, malt liquor, barley wine, happoushu, high-alcohol beer, low-alcohol beer, low-calorie beer, or light beer. In another typical embodiment, the wort is fermented to produce potable ethanol.

The present disclosure also provides an animal feed composition or formulation comprising at least one variant glucoamylase encompassed by the disclosure. Methods of using a glucoamylase enzyme in the production of feeds comprising starch are provided in WO 03/049550 (herein incorporated by reference in its entirety). Briefly, the glucoamylase variant is admixed with a feed comprising starch. The glucoamylase is capable of degrading resistant starch for use by the animal. Other objects and advantages of the present disclosure are apparent from the present specification.

### EXAMPLES

### Assays and Methods

The following assays and methods are used in the examples provided below. The methods used to provide variants are described below. However, it should be noted that different methods may be used to provide variants of a parent enzyme and the invention is not limited to the methods used in the examples. It is intended that any suitable means for making variants and selection of variants may be used.

pNPG Glucoamylase Activity Assay for 96-Well Microtiter Plates

The reagent solutions were: NaAc buffer: 200 mM sodium acetate buffer pH 4.5;

Substrate: 50 mM p-nitrophenyl-α-D-glucopyranoside (Sigma N-1377) in NaAc buffer (0.3 g/20 ml) and stop solution: 800 mM glycine-NaOH buffer pH 10. 30 μl filtered supernatant was placed in a fresh 96-well flat bottom MTP. To each well 50 μl NaAc buffer and 120 μl substrate was added and incubated for 30 minutes at 50° C. (Thermolab systems iEMS Incubator/shaker HT). The reaction was terminated by adding 100 μl stop solution. The absorbance was measured at 405 nm in a MTP-reader (Molecular Devices Spectramax 384 plus) and the activity was calculated using a molar extinction coefficient of 0.011 μM/cm.

Thermal Stability Assay

With a stock dilution of 150 ppm of purified enzyme (in 50 mM NaAc pH 4.0), a 3 ppm dilution was made by adding 6 μl to 294 μl 50 mM NaAc buffer pH 4.5. The diluted sample was equally divided over 2 MTPs. One MTP (initial plate) was incubated for 1 hr at 4° C. and the other MTP (residual plate) was incubated at 64° C. (Thermolab systems iEMS Incubator/Shaker HT) for 1 hr. The residual plate was chilled for 10 min on ice. 60 μl of both the initial plate and the residual plate was added to 120 μl 4% soluble corn starch pH 3.7 and incubated for 2 hrs at 32° C., 900 rpm in 2 separate MTPs (Thermolabsystems iEMS Incubator/Shaker HT). Activity of both plates was measured in the Hexokinase activity assay, using the ethanol application assay described below.

Thermal stability was calculated as % residual activity as follows:

[symbol]

×100%

Hexokinase Activity Assay

Hexokinase cocktail: 10-15 minutes prior to use, 90 ml water was added to a BoatIL container glucose HK R1 (IL test glucose (HK) kit, Instrument Laboratory #182507-40) and gently mixed. 100 μl of Hexokinase cocktail was added to 85 μl of dH₂O. 15 μl of sample was added to the mixtures and incubated for 10 minutes in the dark at room temperature. Absorbance was read at 340 nm in a MTP—reader after 10 minutes. Glucose concentrations were calculated according to a glucose (0-1.6 mg/ml) standard curve.

Ethanol Application—Glucose Release from Corn Starch

8% stock solution: 8 g of soluble corn starch (Sigma #S4180) was suspended in 40 ml dH₂O at room temperature. The slurry was added in portions to 50 ml of boiling dH₂O in a 250 ml flask and cooked for 5 minutes. The starch solution was cooled to 25° C. while stirring and the volume adjusted with remain 10 ml of dH₂O.

Stop solution: 800 mM Glycine-NaOH buffer, pH 10.

4% (m/v) soluble starch working solution: stock solution was diluted (1:1) with 100 mM sodium acetate buffer pH 4.0.

6 μl of 150 ppm purified enzyme was diluted with 294 μl 50 mM NaAc buffer pH 4.0 in a flat bottom 96-well MTP. 60 μl of this dilution was added to 120 μl 4% soluble corn starch pH 4.0 and incubated for 2 hrs at 32° C. 900 rpm (Thermolabsystems iEMS Incubator/Shaker HT). The reaction was stopped by adding 90 μl 4° C.-cold Stop Solution. The sample was placed on ice for 20 minutes. Starch was spun down at 1118×g at 10° C. for 5 minutes (SIGMA 6K15) and 15 μl supernatant was used in the Hexokinase activity assay described above to determine the glucose content.

Data Analysis and Calculation of Performance Index of Ethanol Screening Assay

Protein levels were measured using a microfluidic electrophoresis instrument (Caliper Life Sciences, Hopkinton, Mass., USA). The microfluidic chip and protein samples were prepared according to the manufacturer's instructions (LabChip® HT Protein Express, P/N 760301). Culture supernatants were prepared and stored in 96-well microtiter plates at −20° C. until use, when they were thawed by warming in a 37° C. incubator for 30 minutes. After shaking briefly, 2 μl of each culture sample was transferred to a 96-well PCR plate (Bio-Rad, Hercules, Calif., USA) containing 7 μl samples buffer (Caliper) followed by heating the plate to 90° C. for 5 minutes on a thermostatically controlled plate heater. The plate was allowed to cool before adding 40 μl water to each sample. The plate was then placed in the instrument along with a protein standard supplied and calibrated by the manufacturer. As the proteins move past a focal point in the chip, the fluorescence signal is recorded and the protein concentration is determined by quantitating the signal relative to the signal generated by the calibrated set of protein standards.

After the Caliper protein determination the data is processed in the following way.

The calibration ladders are checked for correctness of the peak pattern. If the calibration ladder that is associated with the run does not suffice, it is replaced by a calibration ladder of an adjacent run. For peak detection, the default settings of the global peak find option of the caliper software are used. The peak of interest is selected at 75 kDA+/−10%. The result is exported to a spreadsheet program and the peak area is related to the corresponding activity (ABS340-blank measurement) in the ethanol screening assay.

With the area and activity numbers of 12 Wild Type samples, a calibration line is made using the “Enzyme Kinetics” equation of the program Grafit Version 5 (Erithacus Software, Horley, UK) in combination with a non-linear fit function. The default settings are used to calculate the Km and Vmax parameters. Based on these two parameters, a Michaelis-Menten reference line is made and the specific activity of each variant is calculated.

Based on the specific activity the performance index (PI) is calculated. The PI of a variant is the quotient “Variant-specific activity/WT-specific activity.” The PI of WT is 1.0 and a variant with a PI>1.0 has a specific activity that is greater than WT.

Purification of TrGA Variants

Concentrated shake flask culture supernatants of expressed TrGA variants were purified in one step by affinity chromatography using an AKTA explorer 100 FPLC system (Amersham Biosciences, Piscataway, N.J.). β-cyclodextrin (Sigma-Aldrich, Zwijndrecht, The Netherlands; 85.608-8) was coupled to epoxy activated Sepharose beads (GE Healthcare, Diegem, Belgium; 17-0480-01) and employed for purification. The column was equilibrated with 25 mM sodium acetate buffer pH 4.3 followed by application of concentrated enzyme sample. Bound variants were eluted from the column with 25 mM sodium acetate buffer pH 4.3 containing 10 mM α-cyclodextrin (Sigma, 28705). Purified samples were analyzed using sulfate-polyacrylamide gel electrophoresis (SDS-PAGE).

Protein Quantification of Purified TrGA Variants

The protein concentration of purified TrGA variants was determined by anion exchange chromatography using an AKTA explorer 100 FPLC system. Purified sample was injected onto a ResourceQ_{—}1 ml column (GE Healthcare) and a linear gradient of 0 to 500 mM NaCl in 25 mM sodium acetate buffer pH 4.3 was applied to elute bound protein. The peak area was determined and the protein concentration was calculated relative to a TrGA standard with know concentration.

Liquefact Assay

Glucose release of the variants was determined on corn mash liquefact from a local ethanol producer in a 6-well plate. Each well of the plate was filled with 6 g of 26% DS liquefact pH 4.3. Subsequently, 300 ppm enzyme and 400 ppm urea was added and 250 μl sample was collected after 2, 4 and 6 hr incubation at 32° C. The sample was centrifuged for 5 minutes at 14.000×g and 50 μl of the supernatant was transferred to an eppendorf tube containing 50 μl of kill solution (1.1 N sulfuric acid) and allowed to stand for 5 minutes. 250 μl of water was added to the tube and then filtered with a 0.22 μm filter plate and injected onto an HPX-87H column as described below.

Evaluation of Performance of TrGA Variant in Ethanol Fermentations

A sample of corn mash liquefact from a local ethanol producer was obtained and diluted in some cases to 26% DS using thin stillage. The pH of the slurry was adjusted to pH 4.3 using 4 N sulfuric acid. A 100 g or 50 g aliquot of mash was put into a 125 ml shake flask and placed into a 32° C. incubator and allowed to equilibrate. After addition of 100 μl 400 ppm urea, 1 ml purified variant at intended concentration or purified TrGA at 2 different concentrations was added to the shake flasks. Finally, 333 μl of a solution of Red Star Red yeast (15 g hydrated for 30 minutes in 45 ml DI water; Lesaffre yeast Corp. Milwaukee, Wis.) was added to each sample. Samples were collected at 5, 21, 28, 48 and 52 hours and analyzed by HPLC (Agilent 1200 series) using an Aminex HPX-87H column (Bio-Rad).

Ethanol and Carbohydrate Determinations

A 2 ml eppendorf centrifuge tube was filled with fermentor beer and cooled on ice for 10 minutes. The sample was centrifuged for 3 minutes at 14.000×g and 500 μl of the supernatant was transferred to a test tube containing 50 μl of kill solution (1.1 N sulfuric acid) and allowed to stand for 5 minutes. 5.0 ml of water was added to the test tube and then filtered into a 0.22 μm filter plate (multiscreen, Millipore, Amsterdam, the Netherlands) and run on HPLC. Column Temperature: 60° C.; mobile phase: 0.01 N sulfuric acid; flow rate 0.6 ml/min; detector: R1; injection volume: 20 μl. The column separates molecules based on charge and molecular weight; DP1 (monosaccharides); DP2 (disaccharides); DP3 (trisaccharides); DP>3 (oligosaccharides sugars having a degree of polymerization greater than 3); succinic acid; lactic acid; glycerol; methanol; ethanol.

### Example 1

### Construction of TrGA Site Evaluation Libraries (SELs) in the pTTT Vector for Expression in Trichoderma reesei

A Trichoderma reesei cDNA sequence (SEQ ID NO: 4) was cloned into pDONR™201 via the Gateway® BP recombination reaction (Invitrogen, Carlsbad, Calif., USA) resulting in the entry vector pDONR-TrGA (FIG. 2). The cDNA sequence (SEQ ID NO: 4) encodes the TrGA signal peptide, the pro-sequence, and the mature protein, including the catalytic domain, linker region and starch binding domain (SEQ ID NO: 1). SEQ ID NO: 4 and SEQ ID NO: 1 are shown in FIGS. 1B and 1B. FIG. 1C illustrates the precursor and mature protein TrGA domains.

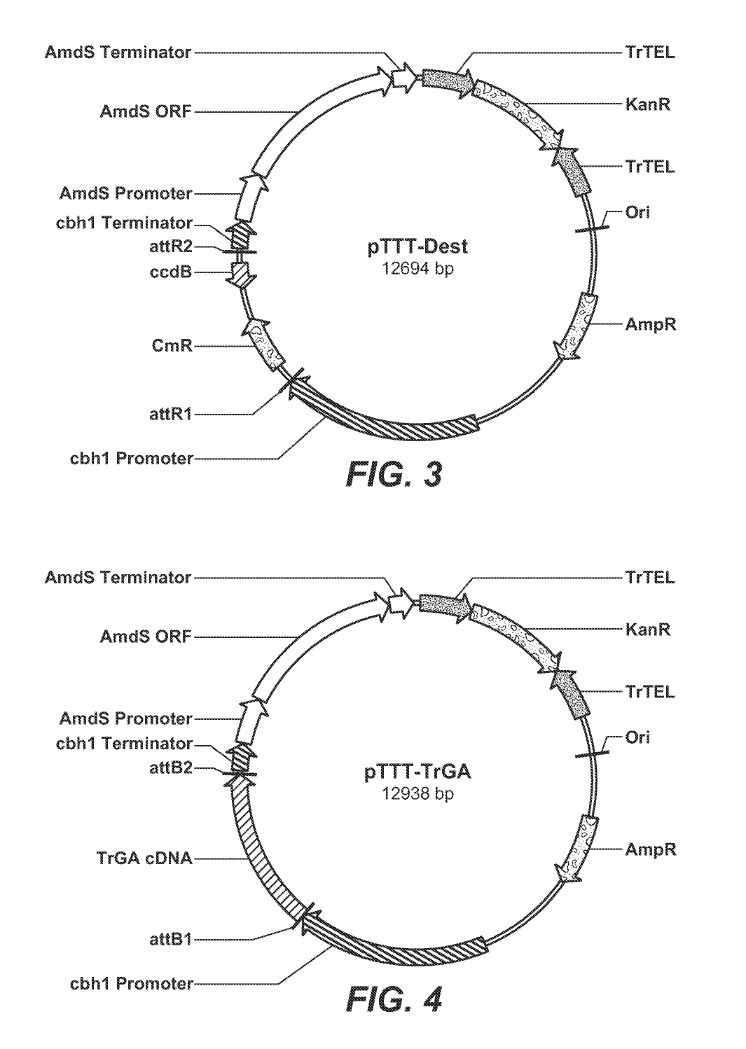

To express the TrGA protein in Trichoderma reesei, the TrGA coding sequence (SEQ ID NO: 4) was cloned into the Gateway compatible destination vector pTTT-Dest (FIG. 3) via the GATEWAY® LR recombination reaction. The expression vector contained the T. reesei cbhI-derived promoter and terminator regions that allowed for strong inducible expression of a gene of interest. The vector also contained the Aspergillus nidulans amdS selective marker that allowed for growth of the transformants on acetamide as a sole nitrogen source. The expression vector also contained T. reesei telomere regions that allowed for non-chromosomal plasmid maintenance in a fungal cell. On the destination pTTT-Dest plasmid, the cbhI promoter and terminator regions were separated by the chloramphenicol resistance gene, Cm^{R}, and the lethal E. coli gene, ccdB, flanked by the bacteriophage lambda-based specific recombination sites attR1, attR2. This configuration allowed for direct selection of recombinants containing the TrGA gene under control of the cbhI regulatory elements in the right orientation via the GATEWAY® LR recombination reaction. The final expression vector pTTT-TrGA is shown in FIG. 4.

SELs were constructed using the pDONR-TrGA entry vector (FIG. 2) as a template and the primers listed in Table 2. All primers used in the mutagenesis experiments contained the triplet NNS(N=A,C,T,G, and S═C or G) at the position that aligns with the codon of the TrGA sequence designed to be mutated (SEQ ID NO: 1), allowing for a random incorporation of nucleotides at the preselected position. Construction of each SEL library started with two independent PCR amplifications on the pDONR-TrGA entry vector: one using the Gateway F (pDONR201-FW) and a specific mutagenesis primer R (Table 2), and the other—the Gateway primer R (pDONR201—RV) and a specific mutagenesis primer F (Table 2). High fidelity PHUSION DNA polymerase (Finnzymes OY, Espoo, Finland) was used in a PCR amplification reaction including 0.2 μM primers. The reactions were carried out for 25 cycles according to the protocol provided by Finnzymes. 1 μl aliquots of the PCR fragments obtained were used as templates for a subsequent fusion PCR reaction together with the Gateway FW and Gateway RV primers (Invitrogen). This PCR amplification, after 22 cycles, produced a population of the full-length linear TrGA DNA fragments randomly mutated at the specific codon position. The fragments were flanked by the Gateway-specific attll, attL2 recombination sites on both ends. The DNA fragments were purified with a CHARGES WITCH® PCR clean-up kit (Invitrogen, Carlsbad USA) and then recombined with 100 ng of the pTTT—destination vector (FIG. 3) using the LR CLONASE™ II enzyme mix according to the protocol supplied by Invitrogen. The recombination products that were generated were transformed into E. coli Max Efficiency DH5a, as described by the supplier (Invitrogen). The final expression constructs pTTT-TrGA with mutations at the desired position were selected by plating bacteria on 2×YT agar plates (16 g/L Bacto Tryptone (Difco), 10 g/L Bacto Yeast Extract (Difco), 5 g/L NaCl, 16 g/L Bacto Agar (Difco)) with 100 vg/ml ampicillin.

96 single colonies from each library were grown for 24 hrs at 37° C. in MTP containing 200 μL 2×YT medium with 100 μg/ml ampicillin. Cultures were used directly to amplify PCR fragments encompassing the region where a specific mutation was introduced. The specific PCR products obtained were sequenced using an ABI3100 sequence analyzer (Applied Biosystems). Each library contained from 15 to 19 different TrGA variants in the final expression vector. These variants were individually transformed into T. reesei, as described below. Libraries are numbered from 1 to 182 referencing the specific amino acid residue in the TrGA sequence that was randomly mutated.

TABLE 2
Primers used to generate TrGA SELs

| AA- | | |
|---|---|---|
| position | F/R | DNA Sequence 5′ to 3′ |
| pDONR201- | F | TCGCGTTAACGCTAGCATGGATCTC |
| | | (SEQ ID NO: 13) |
| pDONR201- | R | TCGCGTTAACGCTAGCATGGATCTC |
| | | (SEQ ID NO: 14) |
| 4 | F | CGTCACCAAGAGGTCTGTTGACNNSTTCATCAGCAC |
| | | CGAGACGCC |
| | | (SEQ ID NO: 15) |
| 4 | R | GTCAACAGACCTCTTGGTGACGTCG |
| | | (SEQ ID NO: 16) |
| 5 | F | CACCAAGAGGTCTGTTGACGACNNSATCAGCACCG |
| | | AGACGCCTATTGC |
| | | (SEQ ID NO: 17) |
| 5 | R | GTCGTCAACAGACCTCTTGGTGAC |
| | | (SEQ ID NO: 18) |
| 10 | F | TGACGACTTCATCAGCACCGAGNNSCCTATTGCA |
| | | CTG |
| | | (SEQ ID NO: 19) |
| 10 | R | CTCGGTGCTGATGAAGTCGTC |
| | | (SEQ ID NO: 20) |
| 12 | F | TCATCAGCACCGAGACGCCTNNSGCACTGAACAATC |
| | | TTCTTTGCA |
| | | (SEQ ID NO: 21) |
| 12 | R | AGGCGTCTCGGTGCTGATGAAGTCG |
| | | (SEQ ID NO: 22) |
| 14 | F | CAGCACCGAGACGCCTATTGCANNSAACAATCTT |
| | | CTT |
| | | (SEQ ID NO: 23) |
| 14 | R | TGCAATAGGCGTCTCGGTGCT |
| | | (SEQ ID NO: 24) |
| 15 | F | CACCGAGACGCCTATTGCACTGNNSAATCTTCTT |
| | | TGC |
| | | (SEQ ID NO: 25) |
| 15 | R | CAGTGCAATAGGCGTCTCGGT |
| | | (SEQ ID NO: 26) |
| 23 | F | CAATCTTCTTTGCAATGTTGGTNNSGATGGATGC |
| | | CGT |
| | | (SEQ ID NO: 27) |
| 23 | R | ACCAACATTGCAAAGAAGATTG |
| | | (SEQ ID NO: 28) |
| 24 | F | TTCTTTGCAATGTTGGTCCTNNSGGATGCCGTGCATT |
| | | CGGCACAT |
| | | (SEQ ID NO: 29) |
| 24 | R | AGGACCAACATTGCAAAGAAGATTG |
| | | (SEQ ID NO: 30) |
| 29 | F | GTCCTGATGGATGCCGTGCANNSGGCACATCAGCTGG |
| | | TGCGGTGA |
| | | (SEQ ID NO: 31) |
| 29 | R | TGCACGGCATCCATCAGGACCAACA |
| | | (SEQ ID NO: 32) |
| 42 | F | TGCGGTGATTGCATCTCCCAGCNNSATTGACCC |
| | | GGAC |
| | | (SEQ ID NO: 33) |
| 42 | R | GCTGGGAGATGCAATCACCGCA |
| | | (SEQ ID NO: 34) |
| 43 | F | TGATTGCATCTCCCAGCACANNSGACCCGGACTACTA |
| | | TTACATGT |
| | | (SEQ ID NO: 35) |
| 43 | R | TGTGCTGGGAGATGCAATCACCGCA |
| | | (SEQ ID NO: 36) |
| 44 | F | TTGCATCTCCCAGCACAATTNNSCCGGACTACTATTA |
| | | CATGTGGA |
| | | (SEQ ID NO: 37) |
| 44 | R | AATTGTGCTGGGAGATGCAATCACC |
| | | (SEQ ID NO: 38) |
| 45 | F | CATCTCCCAGCACAATTGACNNSGACTACTATTACA |
| | | TGTGGACGC |
| | | (SEQ ID NO: 39) |
| 45 | R | GTCAATTGTGCTGGGAGATGCAATC |
| | | (SEQ ID NO: 40) |
| 46 | F | CTCCCAGCACAATTGACCCGNNSTACTATTACATGT |
| | | GGACGCGAGA |
| | | (SEQ ID NO: 41) |
| 46 | R | CGGGTCAATTGTGCTGGGAGATGCA |
| | | (SEQ ID NO: 42) |
| 47 | F | CCAGCACAATTGACCCGGACNNSTATTACATGTGGA |
| | | CGCGAGATA |
| | | (SEQ ID NO: 43) |
| 47 | R | GTCCGGGTCAATTGTGCTGGGAGAT |
| | | (SEQ ID NO: 44) |
| 49 | F | CAATTGACCCGGACTACTATNNSATGTGGACGCGAG |
| | | ATAGCGCTC |
| | | (SEQ ID NO: 45) |
| 49 | R | ATAGTAGTCCGGGTCAATTGTGCTG |
| | | (SEQ ID NO: 46) |
| 51 | F | ACCCGGACTACTATTACATGNNSACGCGAGATAGC |
| | | GCTCTTGTCT |
| | | (SEQ ID NO: 47) |
| 51 | R | CATGTAATAGTAGTCCGGGTCAATT |
| | | (SEQ ID NO: 48) |
| 59 | F | GACGCGAGATAGCGCTCTTGTCNNSAAGAACCTC |
| | | ATC |
| | | (SEQ ID NO: 49) |
| 59 | R | GACAAGAGCGCTATCTCGCGT |
| | | (SEQ ID NO: 50) |
| 60 | F | GCGAGATAGCGCTCTTGTCTTCNNSAACCTCATC |
| | | GAC |
| | | (SEQ ID NO: 51) |
| 60 | R | GAAGACAAGAGCGCTATCTCG |
| | | (SEQ ID NO: 52) |
| 61 | F | AGATAGCGCTCTTGTCTTCAAGNNSCTCATCGAC |
| | | CGC |
| | | (SEQ ID NO: 53) |
| 61 | R | CTTGAAGACAAGAGCGCTATC |
| | | (SEQ ID NO: 54) |
| 65 | F | TGTCTTCAAGAACCTCATCGACNNSTTCACCGAA |
| | | ACG |
| | | (SEQ ID NO: 55) |
| 65 | R | GTCGATGAGGTTCTTGAAGAC |
| | | (SEQ ID NO: 56) |
| 67 | F | CAAGAACCTCATCGACCGCTTCNNSGAAACGTA |
| | | CGAT |
| | | (SEQ ID NO: 57) |
| 67 | R | GAAGCGGTCGATGAGGTTCTT |
| | | (SEQ ID NO: 58) |
| 68 | F | GAACCTCATCGACCGCTTCACCNNSACGTACGA |
| | | TGCG |
| | | (SEQ ID NO: 59) |
| 68 | R | GGTGAAGCGGTCGATGAGGTT |
| | | (SEQ ID NO: 60) |
| 70 | F | TCGACCGCTTCACCGAAACGNNSGATGCGGGCCT |
| | | GCAGCGCCGCA |
| | | (SEQ ID NO: 61) |
| 70 | R | CGTTTCGGTGAAGCGGTCGATGAGG |
| | | (SEQ ID NO: 62) |
| 72 | F | CCGCTTCACCGAAACGTACGATNNSGGCCTGCA |
| | | GCGC |
| | | (SEQ ID NO: 63) |
| 72 | R | ATCGTACGTTTCGGTGAAGCGG |
| | | (SEQ ID NO: 64) |
| 73 | F | CTTCACCGAAACGTACGATGCGNNSCTGCAGCG |
| | | CCGC |
| | | (SEQ ID NO: 65) |
| 73 | R | CGCATCGTACGTTTCGGTGAA |
| | | (SEQ ID NO: 66) |
| 75 | F | AAACGTACGATGCGGGCCTGNNSCGCCGCATCGAG |
| | | CAGTACATTA |
| | | (SEQ ID NO: 67) |
| 75 | R | CAGGCCCGCATCGTACGTTTCGGTG |
| | | (SEQ ID NO: 68) |
| 76 | F | CGTACGATGCGGGCCTGCAGNNSCGCATCGAGCAGT |
| | | ACATTACTG |
| | | (SEQ ID NO: 69) |
| 76 | R | CTGCAGGCCCGCATCGTACGTTTCG |
| | | (SEQ ID NO: 70) |
| 94 | F | CTCTCCAGGGCCTCTCTAACNNSTCGGGCTCCCTCGC |
| | | GGACGGCT |
| | | (SEQ ID NO: 71) |
| 94 | R | GTTAGAGAGGCCCTGGAGAGTGACC |
| | | (SEQ ID NO: 72) |
| 97 | F | GGGCCTCTCTAACCCCTCGGGCNNSCTCGCGGAC |
| | | GGC |
| | | (SEQ ID NO: 73) |
| 97 | R | GCCCGAGGGGTTAGAGAGGCC |
| | | (SEQ ID NO: 74) |
| 98 | F | CCTCTCTAACCCCTCGGGCTCCNNSGCGGACGG |
| | | CTCT |
| | | (SEQ ID NO: 75) |
| 98 | R | GGAGCCCGAGGGGTTAGAGAG |
| | | (SEQ ID NO: 76) |
| 99 | F | CTCTAACCCCTCGGGCTCCCTCNNSGACGGCTCT |
| | | GGT |
| | | (SEQ ID NO: 77) |
| 99 | R | GAGGGAGCCCGAGGGGTTAGA |
| | | (SEQ ID NO: 78) |
| 100 | F | ACCCCTCGGGCTCCCTCGCGNNSGGCTCTGGTCTC |
| | | GGCGAGCCCA |
| | | (SEQ ID NO: 79) |
| 100 | R | CGCGAGGGAGCCCGAGGGGTTAGAG |
| | | (SEQ ID NO: 80) |
| 102 | F | CTCGGGCTCCCTCGCGGACGGCNNSGGTCTCGGC |
| | | GAG |
| | | (SEQ ID NO: 81) |
| 102 | R | GCCGTCCGCGAGGGAGCCCGA |
| | | (SEQ ID NO: 82) |
| 110 | F | TGGTCTCGGCGAGCCCAAGTTTNNSTTGACCCTG |
| | | AAG |
| | | (SEQ ID NO: 83) |
| 110 | R | AAACTTGGGCTCGCCGAGACCA |
| | | (SEQ ID NO: 84) |
| 111 | F | TCTCGGCGAGCCCAAGTTTGAGNNSACCCTGAA |
| | | GCCT |
| | | (SEQ ID NO: 85) |
| 111 | R | CTCAAACTTGGGCTCGCCGAG |
| | | (SEQ ID NO: 86) |
| 113 | F | CGAGCCCAAGTTTGAGTTGACCNNSAAGCCTTTC |
| | | ACC |
| | | (SEQ ID NO: 87) |
| 113 | R | GGTCAACTCAAACTTGGGCTC |
| | | (SEQ ID NO: 88) |
| 114 | F | CCAAGTTTGAGTTGACCCTGNNSCCTTTCACCGGC |
| | | AACTGGGGTC |
| | | (SEQ ID NO: 89) |
| 114 | R | CAGGGTCAACTCAAACTTGGGCTCG |
| | | (SEQ ID NO: 90) |
| 116 | F | TTGAGTTGACCCTGAAGCCTNNSACCGGCAACTGG |
| | | GGTCGACCGCA |
| | | (SEQ ID NO: 91) |
| 116 | R | AGGCTTCAGGGTCAACTCAAACTTG |
| | | (SEQ ID NO: 92) |
| 119 | F | CCCTGAAGCCTTTCACCGGCNNSTGGGGTCGACCG |
| | | CAGCGGGATG |
| | | (SEQ ID NO: 93) |
| 119 | R | GCCGGTGAAAGGCTTCAGGGTCAAC |
| | | (SEQ ID NO: 94) |
| 122 | F | CTTTCACCGGCAACTGGGGTNNSCCGCAGCGGGAT |
| | | GGCCCAGCTC |
| | | (SEQ ID NO: 95) |
| 122 | R | ACCCCAGTTGCCGGTGAAAGGCTTC |
| | | (SEQ ID NO: 96) |
| 125 | F | GCAACTGGGGTCGACCGCAGNNSGATGGCCCAGCT |
| | | CTGCGAGCCA |
| | | (SEQ ID NO: 97) |
| 125 | R | CTGCGGTCGACCCCAGTTGCCGGTG |
| | | (SEQ ID NO: 98) |
| 133 | F | GGATGGCCCAGCTCTGCGAGCCNNSGCCTTGATT |
| | | GGA |
| | | (SEQ ID NO: 99) |
| 133 | R | GGCTCGCAGAGCTGGGCCATCC |
| | | (SEQ ID NO: 100) |
| 137 | F | TGCGAGCCATTGCCTTGATTNNSTACTCAAAGTGGC |
| | | TCATCAACA |
| | | (SEQ ID NO: 101) |
| 137 | R | AATCAAGGCAATGGCTCGCAGAGCT |
| | | (SEQ ID NO: 102) |
| 140 | F | CATTGCCTTGATTGGATACTCANNSTGGCTCAT |
| | | CAAC |
| | | (SEQ ID NO: 103) |
| 140 | R | TGAGTATCCAATCAAGGCAATG |
| | | (SEQ ID NO: 104) |
| 144 | F | TGGATACTCAAAGTGGCTCATCNNSAACAACTAT |
| | | CAG |
| | | (SEQ ID NO: 105) |
| 144 | R | GATGAGCCACTTTGAGTATCC |
| | | (SEQ ID NO: 106) |
| 145 | F | ATACTCAAAGTGGCTCATCAACNNSAACTATCAG |
| | | TCG |
| | | (SEQ ID NO: 107) |
| 145 | R | GTTGATGAGCCACTTTGAGTA |
| | | (SEQ ID NO: 108) |
| 146 | F | CAAAGTGGCTCATCAACAACNNSTATCAGTCGACT |
| | | GTGTCCAACG |
| | | (SEQ ID NO: 109) |
| 146 | R | GTTGTTGATGAGCCACTTTGAGTAT |
| | | (SEQ ID NO: 110) |
| 147 | F | AAAGTGGCTCATCAACAACAACNNSCAGTCGACT |
| | | GTG |
| | | (SEQ ID NO: 111) |
| 147 | R | GTTGTTGTTGATGAGCCACTT |
| | | (SEQ ID NO: 112) |
| 148 | F | GGCTCATCAACAACAACTATNNSTCGACTGTGTCCA |
| | | ACGTCATCT |
| | | (SEQ ID NO: 113) |
| 148 | R | ATAGTTGTTGTTGATGAGCCACTTT |
| | | (SEQ ID NO: 114) |
| 152 | F | CAACAACTATCAGTCGACTGTGNNSAACGTCATC |
| | | TGG |
| | | (SEQ ID NO: 115) |
| 152 | R | CACAGTCGACTGATAGTTGTT |
| | | (SEQ ID NO: 116) |
| 153 | F | CAACTATCAGTCGACTGTGTCCNNSGTCATCTGG |
| | | CCT |
| | | (SEQ ID NO: 117) |
| 153 | R | GGACACAGTCGACTGATAGTT |
| | | (SEQ ID NO: 118) |
| 164 | F | GCCTATTGTGCGCAACGACCTCNNSTATGTTGCC |
| | | CAGT |
| | | (SEQ ID NO: 119) |
| 164 | R | GAGGTCGTTGCGCACAATAGG |
| | | (SEQ ID NO: 120) |
| 169 | F | ACCTCAACTATGTTGCCCAGNNSTGGAACCAAACC |
| | | GGCTTTGACC |
| | | (SEQ ID NO: 121) |
| 169 | R | CTGGGCAACATAGTTGAGGTCGTTG |
| | | (SEQ ID NO: 122) |
| 172 | F | ATGTTGCCCAGTACTGGAACNNSACCGGCTTTGACC |
| | | TCTGGGAAG |
| | | (SEQ ID NO: 123) |
| 172 | R | GTTCCAGTACTGGGCAACATAGTTG |
| | | (SEQ ID NO: 124) |
| 175 | F | AGTACTGGAACCAAACCGGCNNSGACCTCTGGGAA |
| | | GAAGTCAATG |
| | | (SEQ ID NO: 125) |
| 175 | R | GCCGGTTTGGTTCCAGTACTGGGCA |
| | | (SEQ ID NO: 126) |
| 178 | F | ACCAAACCGGCTTTGACCTCNNSGAAGAAGTCAATG |
| | | GGAGCTCAT |
| | | (SEQ ID NO: 127) |
| 178 | R | GAGGTCAAAGCCGGTTTGGTTCCAG |
| | | (SEQ ID NO: 128) |
| 180 | F | CCGGCTTTGACCTCTGGGAANNSGTCAATGGGAGCT |
| | | CATTCTTTA |
| | | (SEQ ID NO: 129) |
| 180 | R | TTCCCAGAGGTCAAAGCCGGTTTGG |
| | | (SEQ ID NO: 130) |
| 181 | F | GCTTTGACCTCTGGGAAGAANNSAATGGGAGCTCAT |
| | | TCTTTACTG |
| | | (SEQ ID NO: 131) |
| 181 | R | TTCTTCCCAGAGGTCAAAGCCGGTT |
| | | (SEQ ID NO: 132) |
| 182 | F | CTTTGACCTCTGGGAAGAAGTCNNSGGGAGCTCATT |
| | | C |
| | | (SEQ ID NO: 133) |
| 182 | R | GACTTCTTCCCAGAGGTCAAAG |
| | | (SEQ ID NO: 134) |
| 204 | F | TGTCGAGGGCGCCACTCTTGCTNNSACTCTTGGCCA |
| | | G |
| | | (SEQ ID NO: 135) |
| 204 | R | AGCAAGAGTGGCGCCCTCGAC |
| | | (SEQ ID NO: 136) |
| 205 | F | CGAGGGCGCCACTCTTGCTGCCNNSCTTGGCCAGTC |
| | | G |
| | | (SEQ ID NO: 137) |
| 205 | R | GGCAGCAAGAGTGGCGCCCTC |
| | | (SEQ ID NO: 138) |
| 208 | F | CTCTTGCTGCCACTCTTGGCNNSTCGGGAAGCGCTTA |
| | | TTCATCTG |
| | | (SEQ ID NO: 139) |
| 208 | R | GCCAAGAGTGGCAGCAAGAGTGGCG |
| | | (SEQ ID NO: 140) |
| 211 | F | CCACTCTTGGCCAGTCGGGANNSGCTTATTCATCTGT |
| | | TGCTCCCC |
| | | (SEQ ID NO: 141) |
| 211 | R | TCCCGACTGGCCAAGAGTGGCAGCA |
| | | (SEQ ID NO: 142) |
| 214 | F | TGGCCAGTCGGGAAGCGCTTATNNSTCTGTTGCTCC |
| | | C |
| | | (SEQ ID NO: 143) |
| 214 | R | ATAAGCGCTTCCCGACTGGCC |
| | | (SEQ ID NO: 144) |
| 216 | F | GTCGGGAAGCGCTTATTCATCTNNSGCTCCCCAGGT |
| | | T |
| | | (SEQ ID NO: 145) |
| 216 | R | AGATGAATAAGCGCTTCCCGA |
| | | (SEQ ID NO: 146) |
| 219 | F | CGCTTATTCATCTGTTGCTCCCNNSGTTTTGTGCTT |
| | | T |
| | | (SEQ ID NO: 147) |
| 219 | R | GGGAGCAACAGATGAATAAGC |
| | | (SEQ ID NO: 148) |
| 228 | F | TGTGCTTTCTCCAACGATTCNNSGTGTCGTCTGGTG |
| | | GATACGTCG |
| | | (SEQ ID NO: 149) |
| 228 | R | GAATCGTTGGAGAAAGCACAAAAC |
| | | CT |
| | | (SEQ ID NO: 150) |
| 229 | F | GTGCTTTCTCCAACGATTCTGGNNSTCGTCTGGTG |
| | | GA |
| | | (SEQ ID NO: 151) |
| 229 | R | CCAGAATCGTTGGAGAAAGCA |
| | | (SEQ ID NO: 152) |
| 230 | F | CTTTCTCCAACGATTCTGGGTGNNSTCTGGTGGAT |
| | | ACG |
| | | (SEQ ID NO: 153) |
| 230 | R | CACCCAGAATCGTTGGAGAAA |
| | | (SEQ ID NO: 154) |
| 231 | F | TCTCCAACGATTCTGGGTGTCGNNSGGTGGATAC |
| | | GTC |
| | | (SEQ ID NO: 155) |
| 231 | R | CGACACCCAGAATCGTTGGAGA |
| | | (SEQ ID NO: 156) |
| 236 | F | GGTGTCGTCTGGTGGATACGTCNNSTCCAACATC |
| | | AACAC |
| | | (SEQ ID NO: 157) |
| 236 | R | GACGTATCCACCAGACGACAC |
| | | (SEQ ID NO: 158) |
| 239 | F | TGGTGGATACGTCGACTCCAACNNSAACACCAAC |
| | | GAG |
| | | (SEQ ID NO: 159) |
| 239 | R | GTTGGAGTCGACGTATCCACC |
| | | (SEQ ID NO: 160) |
| 240 | F | TGGATACGTCGACTCCAACATCNNSACCAACGAG |
| | | GGCA |
| | | (SEQ ID NO: 161) |
| 240 | R | GATGTTGGAGTCGACGTATCCA |
| | | (SEQ ID NO: 162) |
| 241 | F | ATACGTCGACTCCAACATCAACNNSAACGAGGGCAG |
| | | GAC |
| | | (SEQ ID NO: 163) |
| 241 | R | GTTGATGTTGGAGTCGACGTA |
| | | (SEQ ID NO: 164) |
| 242 | F | TCGACTCCAACATCAACACCNNSGAGGGCAGGACTG |
| | | GCAAGGATG |
| | | (SEQ ID NO: 165) |
| 242 | R | GGTGTTGATGTTGGAGTCGACGTAT |
| | | (SEQ ID NO: 166) |
| 243 | F | ACTCCAACATCAACACCAACNNSGGCAGGACTGGCA |
| | | AGGATGTCA |
| | | (SEQ ID NO: 167) |
| 243 | R | GTTGGTGTTGATGTTGGAGTCGACG |
| | | (SEQ ID NO: 168) |
| 244 | F | CTCCAACATCAACACCAACGAGNNSAGGACTGGCA |
| | | AG |
| | | (SEQ ID NO: 169) |
| 244 | R | CTCGTTGGTGTTGATGTTGGAGT |
| | | (SEQ ID NO: 170) |
| 245 | F | ACATCAACACCAACGAGGGCNNSACTGGCAAGGAT |
| | | GTCAACTCCG |
| | | (SEQ ID NO: 171) |
| 245 | R | GCCCTCGTTGGTGTTGATGTTGGAGT |
| | | (SEQ ID NO: 172) |
| 263 | F | TTCCATCCACACCTTCGATCCCNNSCTTGGCTGTG |
| | | AC |
| | | (SEQ ID NO: 173) |
| 263 | R | GGGATCGAAGGTGTGGATGGA |
| | | (SEQ ID NO: 174) |
| 264 | F | CATCCACACCTTCGATCCCAACNNSGGCTGTGACG |
| | | CA |
| | | (SEQ ID NO: 175) |
| 264 | R | GTTGGGATCGAAGGTGTGGAT |
| | | (SEQ ID NO: 176) |
| 265 | F | CCACACCTTCGATCCCAACCTTNNSTGTGACGCAG |
| | | GC |
| | | (SEQ ID NO: 177) |
| 265 | R | AAGGTTGGGATCGAAGGTGTG |
| | | (SEQ ID NO: 178) |
| 268 | F | CGATCCCAACCTTGGCTGTGACNNSGGCACCTTCC |
| | | AGC |
| | | (SEQ ID NO: 179) |
| 268 | R | GTCACAGCCAAGGTTGGGATC |
| | | (SEQ ID NO: 180) |
| 269 | F | TCCCAACCTTGGCTGTGACGCANNSACCTTCCAG |
| | | CCA |
| | | (SEQ ID NO: 181) |
| 269 | R | TGCGTCACAGCCAAGGTTGGG |
| | | (SEQ ID NO: 182) |
| 276 | F | AGGCACCTTCCAGCCATGCAGTNNSAAAGCGCTC |
| | | TCC |
| | | (SEQ ID NO: 183) |
| 276 | R | ACTGCATGGCTGGAAGGTGCC |
| | | (SEQ ID NO: 184) |
| 284 | F | CAAAGCGCTCTCCAACCTCAAGNNSGTTGTCGAC |
| | | TCCT |
| | | (SEQ ID NO: 185) |
| 284 | R | CTTGAGGTTGGAGAGCGCTTT |
| | | (SEQ ID NO: 186) |
| 291 | F | GGTTGTTGTCGACTCCTTCCGCNNSATCTACGGC |
| | | GTG |
| | | (SEQ ID NO: 187) |
| 291 | R | GCGGAAGGAGTCGACAACAAC |
| | | (SEQ ID NO: 188) |
| 292 | F | TTGTCGACTCCTTCCGCTCCNNSTACGGCGTGAAC |
| | | AAGGGCATTC |
| | | (SEQ ID NO: 189) |
| 292 | R | GGAGCGGAAGGAGTCGACAACAACC |
| | | (SEQ ID NO: 190) |
| 294 | F | ACTCCTTCCGCTCCATCTACNNSGTGAACAAGGGCA |
| | | TTCCTGCCG |
| | | (SEQ ID NO: 191) |
| 294 | R | GTAGATGGAGCGGAAGGAGTCGACA |
| | | (SEQ ID NO: 192) |
| 297 | F | GCTCCATCTACGGCGTGAACNNSGGCATTCCTGCCGG |
| | | TGCTGCCG |
| | | (SEQ ID NO: 193) |
| 297 | R | GTTCACGCCGTAGATGGAGCGGAAG |
| | | (SEQ ID NO: 194) |
| 300 | F | CTACGGCGTGAACAAGGGCATTNNSGCCGGTGCTGC |
| | | CG |
| | | (SEQ ID NO: 195) |
| 300 | R | AATGCCCTTGTTCACGCCGTA |
| | | (SEQ ID NO: 196) |
| 301 | F | CGGCGTGAACAAGGGCATTCCTNNSGGTGCTGCCG |
| | | TC |
| | | (SEQ ID NO: 197) |
| 301 | R | AGGAATGCCCTTGTTCACGCC |
| | | (SEQ ID NO: 198) |
| 303 | F | GAACAAGGGCATTCCTGCCGGTNNSGCCGTCGCCA |
| | | TT |
| | | (SEQ ID NO: 199) |
| 303 | R | ACCGGCAGGAATGCCCTTGTT |
| | | (SEQ ID NO: 200) |
| 309 | F | GTGCTGCCGTCGCCATTGGCNNSTATGCAGAGGAT |
| | | GTGTACTACA |
| | | (SEQ ID NO: 201) |
| 309 | R | GCCAATGGCGACGGCAGCACCGGCA |
| | | (SEQ ID NO: 202) |
| 310 | F | CTGCCGTCGCCATTGGCCGGNNSGCAGAGGATGTGT |
| | | ACTACAACG |
| | | (SEQ ID NO: 203) |
| 310 | R | CCGGCCAATGGCGACGGCAGCACCG |
| | | (SEQ ID NO: 204) |
| 311 | F | TGCCGTCGCCATTGGCCGGTATNNSGAGGATGTG |
| | | TAC |
| | | (SEQ ID NO: 205) |
| 311 | R | ATACCGGCCAATGGCGACGGC |
| | | (SEQ ID NO: 206) |
| 313 | F | CCATTGGCCGGTATGCAGAGNNSGTGTACTACAA |
| | | CGGCAACCCTT |
| | | (SEQ ID NO: 207) |
| 313 | F | CCATTGGCCGGTATGCAGAGNNSGTGTACTACAAC |
| | | GGCAACCCTT |
| | | (SEQ ID NO: 208) |
| 313 | R | CTCTGCATACCGGCCAATGGCGACG |
| | | (SEQ ID NO: 209) |
| 313 | R | CTCTGCATACCGGCCAATGGCGACG |
| | | (SEQ ID NO: 210) |
| 314 | F | TTGGCCGGTATGCAGAGGATNNSTACTACAACGGCAA |
| | | CCCTTGGT |
| | | (SEQ ID NO: 211) |
| 314 | R | ATCCTCTGCATACCGGCCAATGGCG |
| | | (SEQ ID NO: 212) |
| 315 | F | GCCGGTATGCAGAGGATGTGNNSTACAACGGCAACC |
| | | CTTGGTATC |
| | | (SEQ ID NO: 213) |
| 315 | R | CACATCCTCTGCATACCGGCCAATG |
| | | (SEQ ID NO: 214) |
| 316 | F | GGTATGCAGAGGATGTGTACNNSAACGGCAACCCTT |
| | | GGTATCTTG |
| | | (SEQ ID NO: 215) |
| 316 | R | GTACACATCCTCTGCATACCGGCCAAT |
| | | (SEQ ID NO: 216) |
| 317 | F | ATGCAGAGGATGTGTACTACNNSGGCAACCCTTGGTA |
| | | TCTTGCTA |
| | | (SEQ ID NO: 217) |
| 317 | F | ATGCAGAGGATGTGTACTACNNSGGCAACCCTTGGTA |
| | | TCTTGCTA |
| | | (SEQ ID NO: 218) |
| 317 | R | GTAGTACACATCCTCTGCATACCGGC |
| | | (SEQ ID NO: 219) |
| 317 | R | GTAGTACACATCCTCTGCATACCGGC |
| | | (SEQ ID NO: 220) |
| 321 | F | TGTACTACAACGGCAACCCTNNSTATCTTGCTACATT |
| | | TGCTGCTG |
| | | (SEQ ID NO: 221) |
| 321 | F | TGTACTACAACGGCAACCCTNNSTATCTTGCTACATT |
| | | TGCTGCTG |
| | | (SEQ ID NO: 222) |
| 321 | R | AGGGTTGCCGTTGTAGTACACATCC |
| | | (SEQ ID NO: 223) |
| 321 | R | AGGGTTGCCGTTGTAGTACACATCC |
| | | (SEQ ID NO: 224) |
| 338 | F | GCAGCTGTACGATGCCATCTACNNSTGGAAGAAGA |
| | | CG |
| | | (SEQ ID NO: 225) |
| 338 | R | GTAGATGGCATCGTACAGCTG |
| | | (SEQ ID NO: 226) |
| 340 | F | ACGATGCCATCTACGTCTGGNNSAAGACGGGCTCC |
| | | ATCACGGTGA |
| | | (SEQ ID NO: 227) |
| 340 | R | CCAGACGTAGATGGCATCGTACAGC |
| | | (SEQ ID NO: 228) |
| 341 | F | ATGCCATCTACGTCTGGAAGNNSACGGGCTCCATCA |
| | | CGGTGACCG |
| | | (SEQ ID NO: 229) |
| 341 | R | CTTCCAGACGTAGATGGCATCGTAC |
| | | AGC |
| | | (SEQ ID NO: 230) |
| 342 | F | ATGCCATCTACGTCTGGAAGAAGNNSGGCTCCA |
| | | TCACG |
| | | (SEQ ID NO: 231) |
| 342 | R | CTTCTTCCAGACGTAGATGGC |
| | | (SEQ ID NO: 232) |
| 344 | F | CTACGTCTGGAAGAAGACGGGCNNSATCACGG |
| | | TGACC |
| | | (SEQ ID NO: 233) |
| 344 | R | GCCCGTCTTCTTCCAGACGTAG |
| | | (SEQ ID NO: 234) |
| 346 | F | CTGGAAGAAGACGGGCTCCATCNNSGTGACCGCC |
| | | ACCTC |
| | | (SEQ ID NO: 235) |
| 346 | R | GATGGAGCCCGTCTTCTTCCA |
| | | (SEQ ID NO: 236) |
| 349 | F | GACGGGCTCCATCACGGTGACCNNSACCTCCCTG |
| | | GCC |
| | | (SEQ ID NO: 237) |
| 349 | R | GGTCACCGTGATGGAGCCCGT |
| | | (SEQ ID NO: 238) |
| 350 | F | GCTCCATCACGGTGACCGCCNNSTCCCTGGCCTTC |
| | | TTCCAGGAGC |
| | | (SEQ ID NO: 239) |
| 350 | R | GGCGGTCACCGTGATGGAGCCCGTC |
| | | (SEQ ID NO: 240) |
| 356 | F | CCACCTCCCTGGCCTTCTTCNNSGAGCTTGTTCCTGG |
| | | CGTGACGG |
| | | (SEQ ID NO: 241) |
| 356 | R | GAAGAAGGCCAGGGAGGTGGCGGTC |
| | | (SEQ ID NO: 242) |
| 359 | F | CCTGGCCTTCTTCCAGGAGCTTNNSCCTGGCGTGA |
| | | CG |
| | | (SEQ ID NO: 243) |
| 359 | R | AAGCTCCTGGAAGAAGGCCAG |
| | | (SEQ ID NO: 244) |
| 361 | F | CTTCTTCCAGGAGCTTGTTCCTNNSGTGACGGCC |
| | | GGG |
| | | (SEQ ID NO: 245) |
| 361 | R | AGGAACAAGCTCCTGGAAGAA |
| | | (SEQ ID NO: 246) |
| 363 | F | AGGAGCTTGTTCCTGGCGTGNNSGCCGGGACCTA |
| | | CTCCAGCAGCT |
| | | (SEQ ID NO: 247) |
| 363 | R | CACGCCAGGAACAAGCTCCTGGAAG |
| | | (SEQ ID NO: 248) |
| 364 | F | GGAGCTTGTTCCTGGCGTGACGNNSGGGACCTACT |
| | | CC |
| | | (SEQ ID NO: 249) |
| 364 | R | CGTCACGCCAGGAACAAGCTC |
| | | (SEQ ID NO: 250) |
| 368 | F | GCGTGACGGCCGGGACCTACNNSAGCAGCTCTTCG |
| | | ACCTTTACCA |
| | | (SEQ ID NO: 251) |
| 368 | R | GTAGGTCCCGGCCGTCACGCCAGGA |
| | | (SEQ ID NO: 252) |
| 369 | F | TGACGGCCGGGACCTACTCCNNSAGCTCTTCGACC |
| | | TTTACCAACA |
| | | (SEQ ID NO: 253) |
| 369 | R | GGAGTAGGTCCCGGCCGTCACGCCA |
| | | (SEQ ID NO: 254) |
| 375 | F | CTCCAGCAGCTCTTCGACCTTTNNSAACATCATC |
| | | AACG |
| | | (SEQ ID NO: 255) |
| 375 | R | AAAGGTCGAAGAGCTGCTGGA |
| | | (SEQ ID NO: 256) |
| 376 | F | GCAGCTCTTCGACCTTTACCNNSATCATCAACGCC |
| | | GTCTCGACAT |
| | | (SEQ ID NO: 257) |
| 376 | R | GGTAAAGGTCGAAGAGCTGCTGGAG |
| | | (SEQ ID NO: 258) |
| 379 | F | TTCGACCTTTACCAACATCATCNNSGCCGTCTCG |
| | | ACA |
| | | (SEQ ID NO: 259) |
| 379 | R | GATGATGTTGGTAAAGGTCGA |
| | | (SEQ ID NO: 260) |
| 382 | F | TACCAACATCATCAACGCCGTCNNSACATACGC |
| | | CGAT |
| | | (SEQ ID NO: 261) |
| 382 | R | GACGGCGTTGATGATGTTGGT |
| | | (SEQ ID NO: 262) |
| 390 | F | GACATACGCCGATGGCTTCCTCNNSGAGGCTGCC |
| | | AAG |
| | | (SEQ ID NO: 263) |
| 390 | R | GAGGAAGCCATCGGCGTATGT |
| | | (SEQ ID NO: 264) |
| 391 | F | ATACGCCGATGGCTTCCTCAGCNNSGCTGCCAAG |
| | | TAC |
| | | (SEQ ID NO: 265) |
| 391 | R | GCTGAGGAAGCCATCGGCGTA |
| | | (SEQ ID NO: 266) |
| 393 | F | CGATGGCTTCCTCAGCGAGGCTNNSAAGTACGTC |
| | | CCC |
| | | (SEQ ID NO: 267) |
| 393 | R | AGCCTCGCTGAGGAAGCCATC |
| | | (SEQ ID NO: 268) |
| 394 | F | TGGCTTCCTCAGCGAGGCTGCCNNSTACGTCCCC |
| | | GCC |
| | | (SEQ ID NO: 269) |
| 394 | R | GGCAGCCTCGCTGAGGAAGCC |
| | | (SEQ ID NO: 270) |
| 395 | F | TCCTCAGCGAGGCTGCCAAGNNSGTCCCCGCCGACG |
| | | GTTCGCTGG |
| | | (SEQ ID NO: 271) |
| 395 | R | CTTGGCAGCCTCGCTGAGGAAGCCA |
| | | (SEQ ID NO: 272) |
| 398 | F | AGGCTGCCAAGTACGTCCCCNNSGACGGTTCGCTGGC |
| | | CGAGCAGTT |
| | | (SEQ ID NO: 273) |
| 398 | R | GGGGACGTACTTGGCAGCCTCGCTG |
| | | (SEQ ID NO: 274) |
| 401 | F | AGTACGTCCCCGCCGACGGTNNSCTGGCCGAGCAG |
| | | TTTGACCGCA |
| | | (SEQ ID NO: 275) |
| 401 | R | ACCGTCGGCGGGGACGTACTTGGCAG |
| | | (SEQ ID NO: 276) |
| 408 | F | CGCTGGCCGAGCAGTTTGACNNSAACAGCGGCACTCC |
| | | GCTGTCTG |
| | | (SEQ ID NO: 277) |
| 408 | R | GTCAAACTGCTCGGCCAGCGAACCG |
| | | (SEQ ID NO: 278) |
| 409 | F | TGGCCGAGCAGTTTGACCGCNNSAGCGGCACTCCGCT |
| | | GTCTGCGC |
| | | (SEQ ID NO: 279) |
| 409 | R | GCGGTCAAACTGCTCGGCCAGCGAA |
| | | (SEQ ID NO: 280) |
| 410 | F | GGCCGAGCAGTTTGACCGCAACNNSGGCACTCCG |
| | | CTG |
| | | (SEQ ID NO: 281) |
| 410 | R | GTTGCGGTCAAACTGCTCGGC |
| | | (SEQ ID NO: 282) |
| 412 | F | AGTTTGACCGCAACAGCGGCNNSCCGCTGTCTGCGC |
| | | TTCACCTGA |
| | | (SEQ ID NO: 283) |
| 412 | R | GCCGCTGTTGCGGTCAAACTGCTCG |
| | | (SEQ ID NO: 284) |
| 415 | F | GCAACAGCGGCACTCCGCTGNNSGCGCTTCACCTGA |
| | | CGTGGTCGT |
| | | (SEQ ID NO: 285) |
| 415 | R | CAGCGGAGTGCCGCTGTTGCGGTCA |
| | | (SEQ ID NO: 286) |
| 417 | F | CAGCGGCACTCCGCTGTCTGCGNNSCACCTGACGT |
| | | GGT |
| | | (SEQ ID NO: 287) |
| 417 | R | CGCAGACAGCGGAGTGCCGCT |
| | | (SEQ ID NO: 288) |
| 418 | F | GCACTCCGCTGTCTGCGCTTNNSCTGACGTGGTCGT |
| | | ACGCCTCGT |
| | | (SEQ ID NO: 289) |
| 418 | R | AAGCGCAGACAGCGGAGTGCCGCTG |
| | | (SEQ ID NO: 290) |
| 421 | F | TGTCTGCGCTTCACCTGACGNNSTCGTACGCCTCGTT |
| | | CTTGACAG |
| | | (SEQ ID NO: 291) |
| 421 | R | CGTCAGGTGAAGCGCAGACAGCGGA |
| | | (SEQ ID NO: 292) |
| 430 | F | GTACGCCTCGTTCTTGACAGCCNNSGCCCGTCGG |
| | | GCT |
| | | (SEQ ID NO: 293) |
| 430 | R | GGCTGTCAAGAACGAGGCGTA |
| | | (SEQ ID NO: 294) |
| 431 | F | CGCCTCGTTCTTGACAGCCACGNNSCGTCGGGC |
| | | TGGC |
| | | (SEQ ID NO: 295) |
| 431 | R | CGTGGCTGTCAAGAACGAGGC |
| | | (SEQ ID NO: 296) |
| 433 | F | TCTTGACAGCCACGGCCCGTNNSGCTGGCATCGTG |
| | | CCCCCCTCGT |
| | | (SEQ ID NO: 297) |
| 433 | R | ACGGGCCGTGGCTGTCAAGAACGAG |
| | | (SEQ ID NO: 298) |
| 436 | F | CCACGGCCCGTCGGGCTGGCNNSGTGCCCCCCTCGT |
| | | GGGCCAACA |
| | | (SEQ ID NO: 299) |
| 436 | R | GCCAGCCCGACGGGCCGTGGCTGTC |
| | | (SEQ ID NO: 300) |
| 442 | F | TGGCATCGTGCCCCCCTCGTGGNNSAACAGCAG |
| | | CGCT |
| | | (SEQ ID NO: 301) |
| 442 | R | CCACGAGGGGGGCACGATGCC |
| | | (SEQ ID NO: 302) |
| 443 | F | CATCGTGCCCCCCTCGTGGGCCNNSAGCAGCGCT |
| | | AGC |
| | | (SEQ ID NO: 303) |
| 443 | R | GGCCCACGAGGGGGGCACGAT |
| | | (SEQ ID NO: 304) |
| 444 | F | CGTGCCCCCCTCGTGGGCCAACNNSAGCGCTAGC |
| | | ACG |
| | | (SEQ ID NO: 305) |
| 444 | R | GTTGGCCCACGAGGGGGGCAC |
| | | (SEQ ID NO: 306) |
| 448 | F | GTGGGCCAACAGCAGCGCTAGCNNSATCCCCT |
| | | CGACG |
| | | (SEQ ID NO: 307) |
| 451 | F | GCAGCGCTAGCACGATCCCCNNSACGTGCTC |
| | | CGGCGCGTCCGTGG |
| | | (SEQ ID NO: 308) |
| 451 | R | GGGGATCGTGCTAGCGCTGCTGTTG |
| | | (SEQ ID NO: 309) |
| 493 | F | CTACACGCCCCTGCCCTGCGCGNNSCCAACCTCC |
| | | GTG |
| | | (SEQ ID NO: 310) |
| 493 | R | CGCGCAGGGCAGGGGCGTGTA |
| | | (SEQ ID NO: 311) |
| 494 | F | CACGCCCCTGCCCTGCGCGACCNNSACCTCCGTG |
| | | GCC |
| | | (SEQ ID NO: 312) |
| 494 | R | GGTCGCGCAGGGCAGGGGCGT |
| | | (SEQ ID NO: 313) |
| 495 | F | GCCCCTGCCCTGCGCGACCCCANNSTCCGTGGCC |
| | | GTC |
| | | (SEQ ID NO: 314) |
| 495 | R | TGGGGTCGCGCAGGGCAGGGG |
| | | (SEQ ID NO: 315) |
| 501 | F | CCCAACCTCCGTGGCCGTCACCNNSCACGAGCTC |
| | | GTGT |
| | | (SEQ ID NO: 316) |
| 501 | R | GGTGACGGCCACGGAGGTTGG |
| | | (SEQ ID NO: 317) |
| 502 | F | AACCTCCGTGGCCGTCACCTTCNNSGAGCTCGTG |
| | | TCG |
| | | (SEQ ID NO: 318) |
| 502 | R | GAAGGTGACGGCCACGGAGGT |
| | | (SEQ ID NO: 319) |
| 503 | F | CTCCGTGGCCGTCACCTTCCACNNSCTCGTGTCGA |
| | | GACA |
| | | (SEQ ID NO: 320) |
| 503 | R | GTGGAAGGTGACGGCCACGGA |
| | | (SEQ ID NO: 321) |
| 508 | F | CTTCCACGAGCTCGTGTCGACANNSTTTGGCCAG |
| | | ACG |
| | | (SEQ ID NO: 322) |
| 508 | R | TGTCGACACGAGCTCGTGGAA |
| | | (SEQ ID NO: 323) |
| 511 | F | GCTCGTGTCGACACAGTTTGGCNNSACGGTCAAGGTG |
| | | (SEQ ID NO: 324) |
| 511 | R | GCCAAACTGTGTCGACACGAG |
| | | (SEQ ID NO: 325) |
| 514 | F | CACAGTTTGGCCAGACGGTCNNSGTGGCGGGCAACG |
| | | CCGCGGCCC |
| | | (SEQ ID NO: 326) |
| 514 | R | GACCGTCTGGCCAAACTGTGTCGAC |
| | | (SEQ ID NO: 327) |
| 517 | F | TGGCCAGACGGTCAAGGTGGCGNNSAACGCCGCGGC |
| | | CCTGGG |
| | | (SEQ ID NO: 328) |
| 517 | R | CGCCACCTTGACCGTCTGGCCAAACTG |
| | | (SEQ ID NO: 329) |
| 518 | F | CCAGACGGTCAAGGTGGCGGGCNNSGCCGCGGCCCT |
| | | GGGCAACT |
| | | (SEQ ID NO: 330) |
| 518 | R | GCCCGCCACCTTGACCGTCTGGCCAAA |
| | | (SEQ ID NO: 331) |
| 519 | F | GACGGTCAAGGTGGCGGGCAACNNSGCGGCCCTGGG |
| | | CAACT |
| | | (SEQ ID NO: 332) |
| 519 | R | GTTGCCCGCCACCTTGACCGTCTGGCC |
| | | (SEQ ID NO: 333) |
| 520 | F | GGTCAAGGTGGCGGGCAACGCCNNSGCCCTGGGCAA |
| | | CTGGA |
| | | (SEQ ID NO: 334) |
| 520 | R | GGCGTTGCCCGCCACCTTGACCGTCTG |
| | | (SEQ ID NO: 335) |
| 525 | F | CAACGCCGCGGCCCTGGGCAACNNSAGCACGAGCGCC |
| | | GCCG |
| | | (SEQ ID NO: 336) |
| 525 | R | GTTGCCCAGGGCCGCGGCGTTGCCCGC |
| | | (SEQ ID NO: 337) |
| 527 | F | CGCGGCCCTGGGCAACTGGAGCNNSAGCGCCGCCGT |
| | | GGCTC |
| | | (SEQ ID NO: 338) |
| 527 | R | GCTCCAGTTGCCCAGGGCCGCGGCGTT |
| | | (SEQ ID NO: 339) |
| 531 | F | CAACTGGAGCACGAGCGCCGCCNNSGCTCTGGACGC |
| | | CGTCA |
| | | (SEQ ID NO: 340) |
| 531 | R | GGCGGCGCTCGTGCTCCAGTTGCCCAG |
| | | (SEQ ID NO: 341) |
| 533 | F | GAGCACGAGCGCCGCCGTGGCTNNSGACGCCGTCAAC |
| | | TATGC |
| | | (SEQ ID NO: 342) |
| 533 | R | AGCCACGGCGGCGCTCGTGCTCCAGTT |
| | | (SEQ ID NO: 343) |
| 535 | F | GAGCGCCGCCGTGGCTCTGGACNNSGTCAACTATGCC |
| | | GATA |
| | | (SEQ ID NO: 344) |
| 535 | R | GTCCAGAGCCACGGCGGCGCTCGTGCT |
| | | (SEQ ID NO: 345) |
| 536 | F | CGCCGCCGTGGCTCTGGACGCCNNSAACTATGCCGAT |
| | | AACC |
| | | (SEQ ID NO: 346) |
| 536 | R | GGCGTCCAGAGCCACGGCGGCGCTCGT |
| | | (SEQ ID NO: 347) |
| 537 | F | CGCCGTGGCTCTGGACGCCGTCNNSTATGCCGAT |
| | | AAC |
| | | (SEQ ID NO: 348) |
| 537 | F | CGCCGTGGCTCTGGACGCCGTCNNSTATGCCGATAAC |
| | | CACCCC |
| | | (SEQ ID NO: 349) |
| 537 | R | GACGGCGTCCAGAGCCACGGCGGCGCT |
| | | (SEQ ID NO: 350) |
| 537 | R | GACGGCGTCCAGAGCCACGGCGGCGCT |
| | | (SEQ ID NO: 351) |
| 538 | F | CGTGGCTCTGGACGCCGTCAACNNSGCCGATAACCACC |
| | | CCC |
| | | (SEQ ID NO: 352) |
| 538 | R | GTTGACGGCGTCCAGAGCCACGGCGGCG |
| | | (SEQ ID NO: 353) |
| 539 | F | GGCTCTGGACGCCGTCAACTATNNSGATAACCACCCC |
| | | CTGT |
| | | (SEQ ID NO: 354) |
| 539 | R | ATAGTTGACGGCGTCCAGAGCCACGGC |
| | | (SEQ ID NO: 355) |
| 540 | F | TCTGGACGCCGTCAACTATGCCNNSAACCACCCCCTG |
| | | TGGATT |
| | | (SEQ ID NO: 356) |
| 540 | R | GGCATAGTTGACGGCGTCCAGAGCCAC |
| | | (SEQ ID NO: 357) |
| 541 | F | GGACGCCGTCAACTATGCCGATNNSCACCCCCTGTGGA |
| | | TTGGG |
| | | (SEQ ID NO: 358) |
| 541 | R | ATCGGCATAGTTGACGGCGTCCAGAGC |
| | | (SEQ ID NO: 359) |
| 545 | F | CTATGCCGATAACCACCCCCTGNNSATTGGGACGGTC |
| | | AACCTC |
| | | (SEQ ID NO: 360) |
| 545 | R | CAGGGGGTGGTTATCGGCATAGTTGAC |
| | | (SEQ ID NO: 361) |
| 546 | F | TGCCGATAACCACCCCCTGTGGNNSGGGACGGTCAA |
| | | CCTCGAG |
| | | (SEQ ID NO: 362) |
| 546 | R | CCACAGGGGGTGGTTATCGGCATAGTT |
| | | (SEQ ID NO: 363) |
| 547 | F | CGATAACCACCCCCTGTGGATTNNSACGGTCAACCTC |
| | | GAGGCT |
| | | (SEQ ID NO: 364) |
| 547 | R | AATCCACAGGGGGTGGTTATCGGCATA |
| | | (SEQ ID NO: 365) |
| 549 | F | CCACCCCCTGTGGATTGGGACGNNSAACCTCGAGGC |
| | | TGGAGAC |
| | | (SEQ ID NO: 366) |
| 549 | R | CGTCCCAATCCACAGGGGGTGGTTATC |
| | | (SEQ ID NO: 367) |
| 551 | F | CCTGTGGATTGGGACGGTCAACNNSGAGGCTGGAGA |
| | | CGTCGTG |
| | | (SEQ ID NO: 368) |
| 551 | R | GTTGACCGTCCCAATCCACAGGGGGTG |
| | | (SEQ ID NO: 369) |
| 561 | F | TGGAGACGTCGTGGAGTACAAGNNSATCAATGTGGG |
| | | CCAAGAT |
| | | (SEQ ID NO: 370) |
| 561 | R | CTTGTACTCCACGACGTCTCCAGCCTC |
| | | (SEQ ID NO: 371) |
| 563 | F | CGTCGTGGAGTACAAGTACATCNNSGTGGGCCAAG |
| | | ATGGCTCC |
| | | (SEQ ID NO: 372) |
| 563 | R | GATGTACTTGTACTCCACGACGTCTCC |
| | | (SEQ ID NO: 373) |
| 567 | F | CAAGTACATCAATGTGGGCCAANNSGGCTCCGTGAC |
| | | CTGGGAG |
| | | (SEQ ID NO: 374) |
| 567 | R | TTGGCCCACATTGATGTACTTGTACTC |
| | | (SEQ ID NO: 375) |
| 569 | F | CATCAATGTGGGCCAAGATGGCNNSGTGACCTGGGA |
| | | GAGTGAT |
| | | (SEQ ID NO: 376) |
| 569 | R | GCCATCTTGGCCCACATTGATGTACTTG |
| | | (SEQ ID NO: 377) |
| 577 | F | CGTGACCTGGGAGAGTGATCCCNNSCACACTTACAC |
| | | GGTTCCT |
| | | (SEQ ID NO: 378) |
| 577 | R | GGGATCACTCTCCCAGGTCACGGAGCC |
| | | (SEQ ID NO: 379) |
| 579 | F | CTGGGAGAGTGATCCCAACCACNNSTACACGGTTCC |
| | | TGCGGTG |
| | | (SEQ ID NO: 380) |
| 579 | R | GTGGTTGGGATCACTCTCCCAGGTCAC |
| | | (SEQ ID NO: 381) |
| 583 | F | TCCCAACCACACTTACACGGTTNNSGCGGTGGCTTG |
| | | TGTGACG |
| | | (SEQ ID NO: 382) |
| 583 | R | AACCGTGTAAGTGTGGTTGGGATCACT |
| | | (SEQ ID NO: 383) |

### Example 2

### Transformation of TrGA SELs into Trichoderma reesei

The SELs were transformed into T. reesei using the PEG protoplast method. The E. coli clones of the SELs confirmed by sequence analysis were grown overnight at 37° C. in deep well microtiter plates (Greiner Art. No. 780271) containing 1200 μl of 2×YT medium with ampicillin (100 μg/ml) and kanamycin (50 μg/ml). Plasmid DNAs were isolated from the cultures using CHEMAGIC® Plasmid Mini Kit (Chemagen—Biopolymer Technologie AG, Baesweiler, Germany) and were transformed individually into a T. reesei host strain derived from RL-P37 bearing four gene deletions (Δcbh1, Δcbh2, Δegl1, Δegl2, i.e., “quad-deleted;” see U.S. Pat. No. 5,847,276, WO 92/06184, and WO 05/001036) using the PEG-Protoplast method (Penttilä et al. (1987) Gene 61:155-164) with the following modifications.

For protoplast preparation, spores were grown for 16-24 hours at 24° C. in Trichoderma Minimal Medium (MM) (20 g/L glucose, 15 g/L KH₂PO₄, pH 4.5, 5 g/L (NH₄)₂SO₄, 0.6 g/L MgSO₄.7H₂O, 0.6 g/L CaCl₂.2H₂O, 1 ml of 1000×T. reesei Trace elements solution {5 g/L FeSO₄.7H₂O, 1.4 g/L ZnSO₄.7H₂O, 1.6 g/L MnSO₄.H₂O, 3.7 g/L CoCl₂.6H₂O}) with shaking at 150 rpm. Germinating spores were harvested by centrifugation and treated with 15 mg/ml of β-D-glucanase-G (Interspex—Art. No. 0439-1) solution to lyse the fungal cell walls. Further preparation of protoplasts was performed by a standard method, as described by Penttila et al. (1987 supra).

The transformation method was scaled down 10 fold. In general, transformation mixtures containing up to 600 ng of DNA and 1-5×10⁵ protoplasts in a total volume of 25 μl were treated with 200 ml of 25% PEG solution, diluted with 2 volumes of 1.2 M sorbitol solution, mixed with 3% selective top agarose MM with acetamide (the same Minimal Medium as mentioned above but (NH₄)₂SO₄ was substituted with 20 mM acetamide) and poured onto 2% selective agarose with acetamide either in 24 well microtiter plates or in a 20×20 cm Q-tray divided in 48 wells. The plates were incubated at 28° C. for 5 to 8 days. Spores from the total population of transformants regenerated on each individual well were harvested from the plates using a solution of 0.85% NaCl, 0.015% Tween 80. Spore suspensions were used to inoculate fermentations in 96 wells MTPs. In the case of 24 well MTPs, an additional plating step on a fresh 24 well MTP with selective acetamide MM was introduced in order to enrich the spore numbers.

### Example 3

### Fermentation of T. reesei Transformants Expressing TrGA Variants in a MTP Format

The tranformants were fermented and the supernatants containing the expressed variant TrGA proteins were tested for various properties. In brief, 96-well filter plates (Corning Art. No. 3505) containing in each well 200 μl of LD-GSM medium (5.0 g/L (NH₄)₂SO₄, 33 g/L1,4-piperazinebis(propanesulfonic acid), pH 5.5, 9.0 g/L Casamino acids, 1.0 g/L KH₂PO₄, 1.0 g/L CaCl₂.2H₂O, 1.0 g/L MgSO₄.7H₂O, 2.5 ml/L of 1000×T. reesei trace elements, 20 g/L Glucose, 10 g/L Sophorose) were inoculated in quadruplicate with spore suspensions of T. reesei transformants expressing TrGA variants (more than 10⁴ spores per well). The plates were incubated at 28° C. with 230 rpm shaking and 80% humidity for 6 days. Culture supernatants were harvested by vacuum filtration. The supernatants were used in different assays for screening of variants with improved properties.

### Example 4

### Preparation of the Whole Broth Samples from GA-Producing Transformants

TrGA producing transformants were initially pre-grown in 250 ml shake flasks containing 30 ml of ProFlo medium. Proflo medium contained: 30 g/L α-lactose, 6.5 g/L (NH₄)₂SO₄, 2 g/L KH₂PO₄, 0.3 g/L MgSO₄.7H₂O, 0.2 g/L CaCl₂.2H₂O, 1 ml/L 1000×trace element salt solution as mentioned above, 2 ml/L 10% Tween 80, 22.5 g/L ProFlo cottonseed flour (Traders protein, Memphis, Tenn.), 0.72 g/L CaCO₃. After two days of growth at 28° C. and 140 rpm, 10% of the Proflo culture was transferred into a 250 ml shake flask containing 30 ml of Lactose Defined Medium. The composition of the Lactose Defined Medium was as follows: 5 g/L (NH₄)₂SO₄, 33 g/L 1,4-Piperazinebis (propanesulfonic acid) buffer, pH 5.5, 9 g/L casamino acids, 4.5 g/L KH₂PO₄, 1.0 g/L MgSO₄.7H₂O, 5 ml/L Mazu DF60-P antifoam (Mazur Chemicals, IL), 1 ml/L of 1000×trace element solution. 40 ml/L of 40% (w/v) lactose solution was added to the medium after sterilization. Shake flasks with the Lactose Defined Medium were incubated at 28° C., 140 rpm for 4-5 days.

Mycelium was removed from the culture samples by centrifugation and the supernatant was analyzed for total protein content (BCA Protein Assay Kit, Pierce Cat. No. 23225) and GA activity, as described above in the Assays and Methods section.

The protein profile of the whole broth samples was determined by SDS-PAGE electrophoresis. Samples of the culture supernatant were mixed with an equal volume of 2× sample loading buffer with reducing agent and separated on NUPAGE® Novex 10% Bis-Tris Gel with MES SDS Running Buffer (Invitrogen, Carlsbad, Calif., USA). Polypeptide bands were visualized in the SDS gel with SIMPLYBLUE SafeStain (Invitrogen, Carlsbad, Calif., USA).

### Example 5

### Variants with Improved Thermal Stability

The parent TrGA molecule had a residual activity between 15 and 44% (day-to-day variation) under the conditions described. The performance index was calculated based on the WT TrGA thermostability of the same batch. The performance indices are the quotients PI=(Variant residual activity)/(WT TrGA residual activity). Using this quotient, a performance index >1 indicates an improved stability. Variants that had a thermal stability performance index of more than 1.0 are shown in the following Table 3.

TABLE 3
Thermal stability screening

| | PI |
|---|---|
| | Thermal |
| Variant | Stability |
| T010F | 1.11 |
| T010G | 1.13 |
| T010M | 1.12 |
| T010Q | 1.06 |
| T010R | 1.06 |
| T010S | 1.24 |
| T042V | 1.31 |
| F059A | 1.03 |
| F059G | 1.07 |
| F059L | 1.06 |
| F059M | 1.12 |
| F059Q | 1.1 |
| F059V | 1.03 |
| N061V | 1.06 |
| E068C | 1.23 |
| E068F | 1.14 |
| E068G | 1.15 |
| E068I | 1.19 |
| E068K | 1.01 |
| E068M | 1.29 |
| E068N | 1.18 |
| E068Q | 1.15 |
| E068W | 1.09 |
| A072E | 1.07 |
| A072Q | 1.02 |
| G073F | 1.44 |
| G073M | 1.01 |
| G073N | 1.1 |
| G073W | 1.36 |
| S097E | 1.08 |
| S097G | 1.03 |
| S097T | 1.17 |
| S097V | 1.03 |
| L098C | 1.07 |
| A099C | 1.07 |
| A099F | 1.02 |
| A099I | 1.14 |
| A099L | 1.09 |
| A099Q | 1.07 |
| A099R | 1.02 |
| A099S | 1.11 |
| A099T | 1.02 |
| S102A | 1.02 |
| K114C | 1.19 |
| K114D | 1.17 |
| K114E | 1.16 |
| K114L | 1.1 |
| K114M | 1.21 |
| K114Q | 1.25 |
| I133V | 1.21 |
| K140D | 1.09 |
| K140Q | 1.06 |
| K140S | 1.05 |
| K140W | 1.04 |
| N144A | 1.11 |
| N144F | 1.06 |
| S152C | 1.04 |
| S152G | 1.09 |
| S152I | 1.09 |
| S152N | 1.12 |
| N153A | 1.28 |
| N153D | 1.06 |
| N153E | 1.29 |
| N153F | 1.16 |
| N153H | 1.01 |
| N153L | 1.06 |
| N153M | 1.27 |
| N153S | 1.31 |
| N153V | 1.34 |
| N153W | 1.19 |
| N182R | 1.02 |
| A204D | 1.02 |
| A204M | 1.08 |
| T205C | 1.02 |
| T205D | 1.06 |
| T205N | 1.09 |
| T205P | 1.17 |
| T205S | 1.04 |
| T205V | 1.06 |
| T205Y | 1.07 |
| S214C | 1.02 |
| S214E | 1.03 |
| S214N | 1.07 |
| S214P | 1.04 |
| S214Q | 1.13 |
| S214T | 1.06 |
| S214V | 1.13 |
| S214W | 1.03 |
| S214Y | 1.04 |
| V216I | 1.13 |
| W228A | 1.01 |
| W228F | 1.12 |
| W228G | 1.06 |
| W228H | 1.05 |
| W228I | 1.06 |
| W228L | 1.14 |
| W228M | 1.04 |
| W228Q | 1.15 |
| W228S | 1.15 |
| W228T | 1.06 |
| W228V | 1.21 |
| W228Y | 1.1 |
| V229A | 1.16 |
| V229D | 1.18 |
| V229E | 1.16 |
| V229F | 1.17 |
| V229G | 1.18 |
| V229H | 1.13 |
| V229I | 1.21 |
| V229L | 1.2 |
| V229M | 1.11 |
| V229N | 1.07 |
| V229P | 1.13 |
| V229Q | 1.1 |
| V229R | 1.07 |
| V229S | 1.12 |
| V229T | 1.11 |
| V229W | 1.07 |
| V229Y | 1.08 |
| S230C | 1.09 |
| S230D | 1.08 |
| S230E | 1.11 |
| S230F | 1.08 |
| S230G | 1.09 |
| S230H | 1.03 |
| S230K | 1.02 |
| S230L | 1.02 |
| S230M | 1.08 |
| S230N | 1.16 |
| S230P | 1.12 |
| S230Q | 1.2 |
| S230R | 1.11 |
| S230T | 1.07 |
| S230V | 1.11 |
| S230Y | 1.05 |
| S231A | 1.06 |
| S231C | 1.07 |
| S231D | 1.18 |
| S231E | 1.1 |
| S231F | 1.14 |
| S231K | 1.09 |
| S231L | 1.15 |
| S231M | 1.09 |
| S231N | 1.13 |
| S231Q | 1.18 |
| S231R | 1.13 |
| S231T | 1.16 |
| S231V | 1.21 |
| S231W | 1.06 |
| S231Y | 1.1 |
| D236A | 1.1 |
| D236C | 1.16 |
| D236E | 1.06 |
| D236F | 1.11 |
| D236G | 1.07 |
| D236H | 1.16 |
| D236I | 1.14 |
| D236K | 1.13 |
| D236L | 1.08 |
| D236M | 1.15 |
| D236N | 1.15 |
| D236P | 1.06 |
| D236R | 1.29 |
| D236T | 1.16 |
| D236V | 1.18 |
| D236Y | 1.07 |
| T241V | 1.05 |
| N242F | 1.03 |
| N242H | 1.01 |
| N263A | 1.18 |
| N263C | 1.14 |
| N263L | 1.02 |
| N263M | 1.1 |
| N263R | 1.04 |
| N263T | 1.07 |
| L264A | 1.03 |
| L264D | 1.2 |
| L264I | 1.14 |
| L264K | 1.24 |
| L264M | 1.15 |
| L264P | 1.02 |
| L264R | 1.02 |
| L264V | 1.04 |
| L264Y | 1.15 |
| G265D | 1.08 |
| G265E | 1.03 |
| G265F | 1.08 |
| G265H | 1.09 |
| G265I | 1.09 |
| G265K | 1.05 |
| G265L | 1.07 |
| G265N | 1.15 |
| G265P | 1.07 |
| G265Q | 1.15 |
| G265R | 1.12 |
| G265S | 1.13 |
| G265T | 1.17 |
| G265V | 1.15 |
| G265W | 1.11 |
| G265Y | 1.1 |
| A268C | 1.16 |
| A268D | 1.2 |
| A268E | 1.1 |
| A268F | 1.07 |
| A268G | 1.11 |
| A268I | 1.12 |
| A268K | 1.11 |
| A268L | 1.11 |
| A268M | 1.18 |
| A268N | 1.1 |
| A268P | 1.13 |
| A268R | 1.15 |
| A268S | 1.09 |
| A268T | 1.14 |
| A268W | 1.05 |
| G269K | 1.01 |
| G269N | 1.08 |
| G269P | 1.08 |
| G269Q | 1.02 |
| G269R | 1.06 |
| D276E | 1.03 |
| D276Q | 1.03 |
| D276S | 1.02 |
| D276V | 1.17 |
| V284C | 1.09 |
| V284E | 1.14 |
| V284G | 1.13 |
| V284I | 1.1 |
| V284Q | 1.08 |
| V284R | 1.09 |
| V284S | 1.06 |
| V284T | 1.08 |
| V284W | 1.08 |
| V284Y | 1.12 |
| S291A | 1.21 |
| S291D | 1.02 |
| S291E | 1.02 |
| S291F | 1.24 |
| S291G | 1.05 |
| S291H | 1.25 |
| S291I | 1.1 |
| S291K | 1.13 |
| S291M | 1.21 |
| S291N | 1.08 |
| S291P | 1.11 |
| S291Q | 1.08 |
| S291R | 1.07 |
| S291T | 1.21 |
| S291V | 1.06 |
| S291W | 1.15 |
| P300A | 1.1 |
| P300N | 1.06 |
| P300Q | 1.05 |
| P300R | 1.02 |
| P300T | 1.07 |
| P300V | 1.02 |
| P300W | 1.11 |
| P300Y | 1.04 |
| A301A | 1.13 |
| A301K | 1.11 |
| A301L | 1.07 |
| A301P | 1.22 |
| A301R | 1.21 |
| A301S | 1.12 |
| A301T | 1.14 |
| A301V | 1.07 |
| A301W | 1.14 |
| A301Y | 1.14 |
| A303E | 1.02 |
| A303I | 1.02 |
| A303L | 1.03 |
| A303Q | 1.01 |
| A311C | 1.05 |
| A311E | 1.06 |
| A311G | 1.11 |
| A311H | 1.09 |
| A311I | 1.04 |
| A311K | 1.13 |
| A311L | 1.07 |
| A311R | 1.07 |
| A311S | 1.01 |
| A311Y | 1.02 |
| V338H | 1.12 |
| V338I | 1.21 |
| V338L | 1.17 |
| V338M | 1.13 |
| V338N | 1.23 |
| V338P | 1.18 |
| V338Q | 1.2 |
| V338S | 1.19 |
| V338Y | 1.1 |
| T342C | 1.06 |
| T342I | 1.04 |
| T342L | 1.17 |
| T342P | 1.08 |
| S344A | 1.05 |
| S344C | 1.16 |
| S344D | 1.02 |
| S344F | 1.1 |
| S344K | 1.12 |
| S344M | 1.26 |
| S344N | 1.16 |
| S344P | 1.2 |
| S344Q | 1.22 |
| S344R | 1.22 |
| S344T | 1.19 |
| S344V | 1.2 |
| S344W | 1.03 |
| T346D | 1.03 |
| T346L | 1.02 |
| T346M | 1.07 |
| T346N | 1.15 |
| T346P | 1.13 |
| T346Q | 1.12 |
| T346S | 1.13 |
| T346V | 1.09 |
| T346W | 1.06 |
| T346Y | 1.07 |
| A349D | 1.15 |
| A349E | 1.13 |
| A349F | 1.09 |
| A349G | 1.12 |
| A349H | 1.11 |
| A349K | 1.14 |
| A349L | 1.16 |
| A349M | 1.07 |
| A349N | 1.09 |
| A349P | 1.03 |
| A349Q | 1.09 |
| A349R | 1.04 |
| A349T | 1.09 |
| A349V | 1.08 |
| A349W | 1.04 |
| A349Y | 1.04 |
| V359Q | 1.02 |
| V359R | 1.13 |
| V359Y | 1.01 |
| G361A | 1.13 |
| G361C | 1.16 |
| G361D | 1.35 |
| G361E | 1.24 |
| G361F | 1.2 |
| G361H | 1.11 |
| G361I | 1.2 |
| G361K | 1.16 |
| G361L | 1.22 |
| G361M | 1.28 |
| G361P | 1.27 |
| G361R | 1.19 |
| G361S | 1.22 |
| G361T | 1.16 |
| G361V | 1.15 |
| G361W | 1.22 |
| G361Y | 1.26 |
| A364C | 1.18 |
| A364D | 1.25 |
| A364E | 1.27 |
| A364F | 1.33 |
| A364G | 1.25 |
| A364K | 1.27 |
| A364L | 1.31 |
| A364M | 1.21 |
| A364Q | 1.19 |
| A364R | 1.28 |
| A364S | 1.23 |
| A364T | 1.23 |
| A364V | 1.23 |
| A364W | 1.23 |
| T375A | 1.17 |
| T375E | 1.03 |
| T375F | 1.06 |
| T375G | 1.05 |
| T375H | 1.13 |
| T375K | 1.1 |
| T375L | 1.03 |
| T375M | 1.17 |
| T375N | 1.2 |
| T375P | 1.15 |
| T375V | 1.16 |
| T375W | 1.11 |
| T375Y | 1.17 |
| N379A | 1.11 |
| N379C | 1.03 |
| N379D | 1.06 |
| N379F | 1.07 |
| N379G | 1.09 |
| N379H | 1.07 |
| N379I | 1.04 |
| N379K | 1.04 |
| N379L | 1.04 |
| N379M | 1.08 |
| N379P | 1.17 |
| N379Q | 1.11 |
| N379R | 1.07 |
| N379T | 1.09 |
| N379V | 1.13 |
| N379W | 1.09 |
| N379Y | 1.06 |
| S382A | 1.06 |
| S382D | 1.04 |
| S382E | 1.05 |
| S382G | 1.06 |
| S382I | 1.07 |
| S382K | 1.03 |
| S382N | 1.07 |
| S382P | 1.1 |
| S382R | 1.05 |
| S382T | 1.02 |
| S382Y | 1.04 |
| S390M | 1.06 |
| S390Q | 1.02 |
| S390R | 1.02 |
| E391L | 1.04 |
| E391R | 1.02 |
| E391S | 1.02 |
| E391W | 1.04 |
| E391Y | 1.02 |
| A393D | 1.11 |
| A393E | 1.03 |
| A393F | 1.09 |
| A393G | 1.12 |
| A393H | 1.05 |
| A393I | 1.06 |
| A393K | 1.05 |
| A393L | 1.17 |
| A393M | 1.07 |
| A393N | 1.11 |
| A393Q | 1.02 |
| A393R | 1.03 |
| A393S | 1.13 |
| A393T | 1.08 |
| A393V | 1.09 |
| A393W | 1.1 |
| A393Y | 1.12 |
| K394A | 1.08 |
| K394C | 1.06 |
| K394E | 1.07 |
| K394F | 1.09 |
| K394G | 1.05 |
| K394H | 1.08 |
| K394L | 1.08 |
| K394M | 1.06 |
| K394Q | 1.07 |
| K394R | 1.06 |
| K394T | 1.03 |
| K394V | 1.02 |
| S410E | 1.05 |
| S410H | 1.11 |
| S410I | 1.06 |
| S410K | 1.11 |
| S410L | 1.05 |
| S410M | 1.02 |
| S410N | 1.1 |
| S410Q | 1.15 |
| S410R | 1.18 |
| S410T | 1.04 |
| S410V | 1.13 |
| L417I | 1.04 |
| L417K | 1.2 |
| L417M | 1.05 |
| L417Q | 1.04 |
| L417R | 1.2 |
| L417V | 1.07 |
| L417Y | 1.01 |
| T430A | 1.05 |
| T430E | 1.02 |
| T430F | 1.06 |
| T430H | 1.1 |
| T430I | 1.04 |
| T430K | 1.08 |
| T430M | 1.17 |
| T430N | 1.13 |
| T430Q | 1.05 |
| T430R | 1.13 |
| T430S | 1.17 |
| T430V | 1.05 |
| A431I | 1.03 |
| A431N | 1.03 |
| A431P | 1.08 |
| A431R | 1.08 |
| A431V | 1.03 |
| R433A | 1.13 |
| R433C | 1.24 |
| R433E | 1.22 |
| R433F | 1.17 |
| R433G | 1.23 |
| R433K | 1.12 |
| R433L | 1.23 |
| R433M | 1.1 |
| R433N | 1.28 |
| R433S | 1.23 |
| R433V | 1.28 |
| R433W | 1.16 |
| R433Y | 1.18 |
| I436E | 1.07 |
| I436F | 1.02 |
| I436G | 1.09 |
| I436H | 1.2 |
| I436K | 1.14 |
| I436P | 1.15 |
| I436R | 1.16 |
| I436S | 1.17 |
| I436T | 1.18 |
| I436V | 1.12 |
| I436Y | 1.05 |
| A442N | 1.06 |
| A442R | 1.04 |
| A442T | 1.09 |
| S444E | 1.04 |
| S444K | 1.07 |
| S444M | 1.05 |
| S444Q | 1.04 |
| T448A | 1.02 |
| T448E | 1.09 |
| T448F | 1.12 |
| T448I | 1.12 |
| T448L | 1.09 |
| T448M | 1.11 |
| T448Q | 1.17 |
| T448R | 1.09 |
| T448S | 1.07 |
| T448V | 1.13 |
| T448W | 1.01 |
| T448Y | 1.08 |
| S451E | 1.04 |
| S451H | 1.18 |
| S451K | 1.08 |
| S451L | 1.01 |
| S451Q | 1.06 |
| S451T | 1.02 |
| T493I | 1.15 |
| T495K | 1.2 |
| T495R | 1.1 |
| T495S | 1.23 |
| E503A | 1.43 |
| E503C | 1.39 |
| E503S | 1.02 |
| E503T | 1.04 |
| E503V | 1.68 |
| Q508H | 1.19 |
| Q508R | 1.29 |
| Q508S | 1.13 |
| Q511A | 1.11 |
| Q511D | 1.12 |
| Q511H | 1.33 |
| Q511N | 1.14 |
| Q511S | 1.15 |
| N518S | 1.17 |
| A519E | 1.11 |
| A519K | 1.33 |
| A519R | 1.32 |
| A519T | 1.06 |
| A519V | 1.07 |
| A519Y | 1.29 |
| A520C | 1.1 |
| A520L | 1.06 |
| A520P | 1.06 |
| T527A | 1.08 |
| T527V | 1.12 |
| V531L | 1.21 |
| A535D | 1.12 |
| A535K | 1.24 |
| A535N | 1.37 |
| A535P | 1.59 |
| A535R | 1.26 |
| V536I | 1.02 |
| V536R | 1.1 |
| N537W | 1.16 |
| A539E | 1.32 |
| A539H | 1.17 |
| A539M | 1.05 |
| A539R | 1.36 |
| A539S | 1.3 |
| N563A | 1.14 |
| N563C | 1.49 |
| N563E | 1.44 |
| N563I | 1.65 |
| N563K | 1.77 |
| N563L | 1.6 |
| N563Q | 1.29 |
| N563T | 1.31 |
| N563V | 1.53 |
| N577A | 1.19 |
| N577K | 1.23 |
| N577P | 1.41 |
| N577R | 1.31 |
| N577V | 1.12 |

Table 3 includes those variants that, when tested, showed an increased performance index over the parent glucoamylase. These included the following sites: 10, 42, 59, 61, 68, 72, 73, 97, 98, 99, 102, 114, 133, 140, 144, 152, 153, 182, 204, 205, 214, 216, 228, 229, 230, 231, 236, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 417, 430, 431, 433, 436, 442, 444, 448, 451, 493, 495, 503, 508, 511, 518, 519, 520, 527, 531, 535, 536, 537, 539, 563, and 577. The sites with substitutions showing the highest increase (PI≧1.20) included: T10S, T42V, E68C, E68M, G73F, G73W, K114M, K114Q, I133V, N153A, N153E, N153M, N153S, N153V, W228V, V229I, V229L, S230Q, S231V, D236R, L264D, L264K, A268D, S291A, S291F, S291H, S291M, S291T, A301P, A301R, V338I, V338N, V338Q, S344M, S344P, S344Q, S344R, S344V, G361D, G361E, G361F, G361I, G361L, G361M, G361P, G361S, G361W, G361Y, A364D, A364E, A364F, A364G, A364K, A364L, A364M, A364R, A364S, A364T, A364V, A364W, T375N, L417K, L417R, R433c, R433E, R433G, R433L, R433N, R433S, R433V, I436H, T495K, T495S, E503A, E503C, E503V, Q508R, Q511H, A519K, A519R, A519Y, V531L, A535K, A535N, A535P, A535R, A539E, A539R, A539S, N563C, N563E, N563I, N563K, N563L, N563Q, N563T, N563V, N577K, N577P, and N577R.

### Example 6

### Variants with Improved Specific Activity (SA) in an Ethanol Screening Assay

Variants were tested in an ethanol screening assay using the assays described above. Table 4 shows the results of the screening assay for variants with a Performance Index (PI)>1.0 compared to the parent TrGA PI. The PI is calculated from the specific activities (activity/mg enzyme) of the WT and the variant enzymes. It is the quotient “Variant-specific activity/WT-specific activity.” The PI of the specific activity for the wild type TrGA was 1.0 and a variant with a PI>1.0 had a specific activity that was greater than the parent TrGA. The specific activity was the activity measured by the ethanol screening assay divided by the results obtained in the Caliper assay described above.

TABLE 4
Ethanol screening

| | PI |
|---|---|
| | Specific |
| Variant | Activity |
| T010D | 1.09 |
| T010F | 1.06 |
| T010G | 1.12 |
| T010K | 1.05 |
| T010L | 1.07 |
| T010M | 1.05 |
| T010P | 1.05 |
| T010R | 1.08 |
| T010S | 1.09 |
| L014E | 1.03 |
| L014H | 1.05 |
| N015D | 1.02 |
| P023A | 1.16 |
| P023F | 1.13 |
| P023N | 1.05 |
| F059A | 1.17 |
| F059F | 1.05 |
| F059G | 1.05 |
| K060F | 1.06 |
| K060H | 1.03 |
| N061D | 1.05 |
| N061I | 1.21 |
| N061L | 1.18 |
| N061Q | 1.08 |
| N061V | 1.11 |
| N061W | 1.02 |
| R065A | 1.17 |
| R065C | 1.08 |
| R065G | 1.08 |
| R065I | 1.11 |
| R065K | 1.09 |
| R065M | 1.07 |
| R065S | 1.12 |
| R065V | 1.14 |
| R065Y | 1.01 |
| T067C | 1.14 |
| T067I | 1.13 |
| T067K | 1.05 |
| T067M | 1.22 |
| E068I | 1.06 |
| E068M | 1.1 |
| E068W | 1.03 |
| A072E | 1.11 |
| A072G | 1.02 |
| A072L | 1.03 |
| A072M | 1.11 |
| A072Q | 1.1 |
| A072R | 1.1 |
| A072W | 1.06 |
| A072Y | 1.3 |
| G073C | 1.02 |
| G073L | 1.07 |
| G073W | 1.03 |
| S097F | 1.11 |
| S097M | 1.11 |
| S097N | 1.23 |
| S097P | 1.18 |
| S097R | 1.07 |
| S097V | 1.12 |
| S097W | 1.09 |
| S097Y | 1.18 |
| L098H | 1.04 |
| L098M | 1.09 |
| A099C | 1.07 |
| A099L | 1.01 |
| A099M | 1.03 |
| A099N | 1.11 |
| A099P | 1.08 |
| S102A | 1.2 |
| S102C | 1.04 |
| S102I | 1.04 |
| S102L | 1.05 |
| S102M | 1.25 |
| S102N | 1.19 |
| S102R | 1.21 |
| S102V | 1.07 |
| S102W | 1.06 |
| S102Y | 1.1 |
| E110Q | 1.02 |
| E110S | 1.07 |
| E110W | 1.11 |
| L113E | 1.15 |
| L113N | 1.08 |
| I133K | 1.04 |
| I133R | 1.16 |
| I133S | 1.08 |
| I133T | 1.29 |
| K140A | 1.04 |
| K140E | 1.04 |
| K140F | 1.03 |
| K140H | 1.14 |
| K140L | 1.1 |
| K140M | 1.11 |
| K140N | 1.15 |
| K140Q | 1.08 |
| K140R | 1.12 |
| K140S | 1.13 |
| K140V | 1.15 |
| K140W | 1.07 |
| K140Y | 1.06 |
| N144C | 1.05 |
| N144D | 1.15 |
| N144E | 1.16 |
| N144I | 1.13 |
| N144K | 1.05 |
| N145A | 1.07 |
| N145C | 1.09 |
| N145E | 1.03 |
| N145I | 1.2 |
| N145K | 1.05 |
| N145L | 1.03 |
| N145M | 1.07 |
| N145Q | 1.14 |
| N145R | 1.11 |
| N145V | 1.12 |
| N145W | 1.14 |
| N145Y | 1.05 |
| Y147A | 1.02 |
| Y147M | 1.02 |
| Y147R | 1.12 |
| S152H | 1.08 |
| S152M | 1.1 |
| N153C | 1.09 |
| N153D | 1.2 |
| N153K | 1.13 |
| N153L | 1.12 |
| N153W | 1.07 |
| N153Y | 1.13 |
| N164A | 1.02 |
| N164G | 1.03 |
| N182C | 1.12 |
| N182E | 1.13 |
| N182K | 1.07 |
| N182P | 1.01 |
| N182R | 1.03 |
| A204C | 1.04 |
| A204D | 1.09 |
| A204G | 1.02 |
| A204I | 1.06 |
| A204M | 1.09 |
| A204Q | 1.09 |
| A204T | 1.05 |
| T205A | 1.03 |
| T205D | 1.03 |
| T205H | 1.03 |
| T205I | 1.05 |
| T205K | 1.09 |
| T205M | 1.05 |
| T205N | 1.09 |
| T205P | 1.17 |
| T205Q | 1.25 |
| T205S | 1.1 |
| T205V | 1.06 |
| T205W | 1.05 |
| T205Y | 1.18 |
| S214P | 1.08 |
| S214T | 1.07 |
| V216C | 1.08 |
| V216G | 1.05 |
| V216H | 1.03 |
| V216K | 1.02 |
| V216N | 1.13 |
| V216Y | 1.09 |
| Q219D | 1.05 |
| Q219G | 1.06 |
| Q219H | 1.03 |
| Q219N | 1.08 |
| Q219P | 1.16 |
| Q219S | 1.29 |
| W228A | 1.2 |
| W228F | 1.22 |
| W228G | 1.17 |
| W228H | 1.33 |
| W228I | 1.18 |
| W228L | 1.12 |
| W228M | 1.35 |
| W228T | 1.19 |
| V229E | 1.01 |
| V229I | 1.02 |
| V229M | 1.03 |
| V229N | 1.01 |
| V229Q | 1.02 |
| S230C | 1.23 |
| S230D | 1.13 |
| S230E | 1.1 |
| S230F | 1.63 |
| S230G | 1.77 |
| S230H | 1.05 |
| S230I | 1.18 |
| S230K | 1.04 |
| S230L | 1.2 |
| S230N | 1.23 |
| S230P | 1.13 |
| S230Q | 1.2 |
| S230R | 1.84 |
| S230T | 1.11 |
| S230V | 1.12 |
| S230Y | 1.08 |
| S231C | 1.13 |
| S231D | 1.08 |
| S231F | 1.17 |
| S231L | 1.29 |
| S231M | 1.08 |
| S231N | 1.04 |
| S231Q | 1.05 |
| S231R | 1.02 |
| S231V | 1.07 |
| S231Y | 1.07 |
| D236F | 1.14 |
| D236G | 1.05 |
| D236L | 1.11 |
| D236M | 1.07 |
| D236N | 1.03 |
| D236P | 1.06 |
| D236S | 1.03 |
| D236T | 1.14 |
| D236V | 1.04 |
| I239M | 1.04 |
| I239Q | 1.08 |
| I239S | 1.11 |
| I239V | 1.52 |
| I239W | 1.02 |
| I239Y | 1.25 |
| T241C | 1.07 |
| T241E | 1.03 |
| T241H | 1.1 |
| T241L | 1.04 |
| T241M | 1.05 |
| T241P | 1.02 |
| T241S | 1.08 |
| T241V | 1.05 |
| N242C | 1.08 |
| N242F | 1.06 |
| N242M | 1.04 |
| N242T | 1.08 |
| N242V | 1.03 |
| N242W | 1.05 |
| N263H | 1.05 |
| N263K | 1.02 |
| N263P | 1.4 |
| L264A | 1.04 |
| L264C | 1.08 |
| L264E | 1.16 |
| L264F | 1.03 |
| L264S | 1.05 |
| G265E | 1.1 |
| G265H | 1.12 |
| G265I | 1.06 |
| G265K | 1.03 |
| G265R | 1.06 |
| G265T | 1.1 |
| A268C | 1.5 |
| A268D | 1.14 |
| A268E | 1.18 |
| A268F | 1.15 |
| A268G | 1.35 |
| A268I | 1.15 |
| A268K | 1.23 |
| A268L | 1.06 |
| A268P | 1.08 |
| A268R | 1.14 |
| A268T | 1.18 |
| A268W | 1.05 |
| G269E | 1.01 |
| D276S | 1.01 |
| V284R | 1.06 |
| V284T | 1.05 |
| V284Y | 1.07 |
| S291A | 1.26 |
| S291E | 1.09 |
| S291F | 1.13 |
| S291H | 1.13 |
| S291K | 1.07 |
| S291N | 1.04 |
| S291P | 1.12 |
| S291W | 1.04 |
| P300K | 1.1 |
| P300P | 1.12 |
| P300R | 1.11 |
| A301A | 1.01 |
| A301E | 1.09 |
| A301K | 1.09 |
| A301L | 1.05 |
| A301P | 1.02 |
| A301S | 1.03 |
| A301W | 1.04 |
| A303C | 1.06 |
| A303D | 1.04 |
| A303F | 1.09 |
| A303H | 1.05 |
| A303I | 1.09 |
| A303K | 1.02 |
| A303L | 1.05 |
| A303N | 1.04 |
| A303R | 1.1 |
| A303T | 1.11 |
| A303V | 1.04 |
| A303W | 1.15 |
| A303Y | 1.07 |
| A311N | 1.04 |
| A311P | 1.09 |
| A311Q | 1.19 |
| A311S | 1.01 |
| A311Y | 1.06 |
| V338P | 1.04 |
| V338Q | 1.12 |
| V338S | 1.14 |
| V338Y | 1.05 |
| T342N | 1.06 |
| T342V | 1.23 |
| S344A | 1.05 |
| S344T | 1.01 |
| T346G | 1.14 |
| T346H | 1.07 |
| T346M | 1.06 |
| T346N | 1.09 |
| T346P | 1.07 |
| T346Q | 1.04 |
| T346Y | 1.06 |
| A349L | 1.02 |
| V359I | 1.14 |
| V359K | 1.08 |
| V359M | 1.09 |
| V359N | 1.02 |
| V359Q | 1.14 |
| V359R | 1.15 |
| V359W | 1.04 |
| G361H | 1.11 |
| G361L | 1.04 |
| G361R | 1.04 |
| A364M | 1.05 |
| A364W | 1.07 |
| T375C | 1.12 |
| T375D | 1.01 |
| T375E | 1.02 |
| T375H | 1.05 |
| T375V | 1.04 |
| T375W | 1.02 |
| T375Y | 1.05 |
| N379A | 1.05 |
| N379C | 1.1 |
| N379D | 1.05 |
| N379G | 1.07 |
| N379I | 1.01 |
| N379M | 1.04 |
| N379P | 1.06 |
| N379S | 1.01 |
| S382A | 1.02 |
| S382N | 1.02 |
| S382P | 1.1 |
| S382S | 1.09 |
| S382V | 1.1 |
| S382W | 1.1 |
| S390A | 1.05 |
| S390Y | 1.03 |
| E391A | 1.17 |
| E391E | 1.1 |
| E391I | 1.13 |
| E391K | 1.18 |
| E391L | 1.18 |
| E391M | 1.05 |
| E391Q | 1.04 |
| E391R | 1.08 |
| E391V | 1.05 |
| E391W | 1.12 |
| E391Y | 1.08 |
| A393E | 1.05 |
| A393G | 1.14 |
| A393H | 1.1 |
| A393I | 1.07 |
| A393K | 1.09 |
| A393L | 1.12 |
| A393M | 1.07 |
| A393N | 1.18 |
| A393Q | 1.02 |
| A393R | 1.09 |
| A393S | 1.1 |
| A393T | 1.13 |
| A393V | 1.16 |
| A393W | 1.04 |
| A393Y | 1.03 |
| K394A | 1.11 |
| K394H | 1.11 |
| K394K | 1.08 |
| K394L | 1.01 |
| K394M | 1.14 |
| K394Q | 1.09 |
| K394R | 1.19 |
| K394S | 1.22 |
| K394T | 1.08 |
| K394V | 1.05 |
| K394W | 1.13 |
| S410E | 1.01 |
| S410H | 1.06 |
| S410N | 1.04 |
| L417A | 1.12 |
| L417D | 1.19 |
| L417E | 1.1 |
| L417F | 1.08 |
| L417G | 1.19 |
| L417I | 1.1 |
| L417K | 1.02 |
| L417Q | 1.04 |
| L417R | 1.3 |
| L417S | 1.05 |
| L417T | 1.1 |
| L417V | 1.21 |
| L417W | 1.05 |
| L417Y | 1.1 |
| H418E | 1.01 |
| H418M | 1.12 |
| T430A | 1.19 |
| T430E | 1.15 |
| T430F | 1.09 |
| T430G | 1.16 |
| T430H | 1.15 |
| T430I | 1.06 |
| T430K | 1.24 |
| T430M | 1.16 |
| T430N | 1.07 |
| T430Q | 1.15 |
| T430R | 1.04 |
| T430V | 1.09 |
| A431C | 1.04 |
| A431E | 1.08 |
| A431H | 1.11 |
| A431I | 1.2 |
| A431L | 1.21 |
| A431M | 1.12 |
| A431Q | 1.22 |
| A431R | 1.11 |
| A431S | 1.09 |
| A431W | 1.04 |
| A431Y | 1.13 |
| R433A | 1.09 |
| R433M | 1.17 |
| R433W | 1.06 |
| R433Y | 1.22 |
| A442A | 1.13 |
| S444K | 1.03 |
| S444M | 1.13 |
| S444N | 1.04 |
| S444P | 1.08 |
| S444Q | 1.08 |
| S444R | 1.03 |
| S444T | 1.15 |
| S444V | 1.14 |
| S444W | 1.16 |
| T448F | 1.02 |
| T448G | 1.08 |
| T448I | 1.1 |
| T448P | 1.08 |
| T448Q | 1.04 |
| T448V | 1.04 |
| T451K | 1.29 |
| T493C | 1.05 |
| T493M | 1.19 |
| T493N | 1.07 |
| T493Q | 1.03 |
| T493Y | 1.07 |
| P494H | 1.03 |
| P494I | 1.12 |
| P494M | 1.12 |
| P494N | 1.16 |
| P494Q | 1.02 |
| P494W | 1.07 |
| T495M | 1.49 |
| T495P | 1.04 |
| T495R | 1.15 |
| H502A | 1.16 |
| H502M | 1.13 |
| H502S | 1.15 |
| H502V | 1.1 |
| E503C | 1.05 |
| E503D | 1.06 |
| E503H | 1.01 |
| E503S | 1.1 |
| E503W | 1.04 |
| Q508N | 1.11 |
| Q508P | 1.07 |
| Q508Y | 1.09 |
| Q511C | 1.07 |
| Q511G | 1.06 |
| Q511H | 1.05 |
| Q511I | 1.1 |
| Q511K | 1.09 |
| Q511T | 1.04 |
| Q511V | 1.04 |
| N518P | 1.13 |
| N518T | 1.02 |
| A519I | 1.21 |
| A520C | 1.38 |
| A520E | 1.16 |
| A520L | 1.46 |
| A520P | 1.5 |
| A520Q | 1.05 |
| A520R | 1.06 |
| A520W | 1.07 |
| V531A | 1.02 |
| V531L | 1.04 |
| V531N | 1.19 |
| V531R | 1.06 |
| V531S | 1.08 |
| V531T | 1.19 |
| A535E | 1.19 |
| A535F | 1.06 |
| A535G | 1.02 |
| A535K | 1.07 |
| A535L | 1.02 |
| A535N | 1.04 |
| A535P | 1.14 |
| A535R | 1.22 |
| A535S | 1.06 |
| A535T | 1.04 |
| A535V | 1.04 |
| A535W | 1.09 |
| A535Y | 1.13 |
| V536C | 1.09 |
| V536E | 1.09 |
| V536I | 1.04 |
| V536L | 1.07 |
| V536M | 1.2 |
| V536Q | 1.02 |
| V536S | 1.05 |
| A539E | 1.08 |
| A539M | 1.03 |
| A539S | 1.02 |
| A539W | 1.06 |
| A539R | 1.22 |

Table 4 provides the variants having a performance index of at least 1.0. These included

the following sites: 10, 14, 15, 23, 59, 60, 61, 65, 67, 68, 72, 73, 97, 98, 99, 102, 110, 113, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 417, 418, 430, 431, 433, 442, 444, 448, 451, 493, 494, 495, 502, 503, 508, 511, 518, 519, 520, 531, 535, 536, and 539. The sites showing the highest specific activity (PI 1.20) included: N061I, T067M, A072Y, S097N, S102A, S102M, S102R, I133T, N145I, N153D, T205Q, Q219S, W228A, W228F, W228H, W228M, S230C, S230F, S230G, S230L, S230N, S230Q, S230R, S231L, I239V, I239Y, N263P, A268C, A268G, A268K, S291A, T342V, K394S, L417R, L417V, T430K, A431I, A431L, A431Q, R433Y, T451K, T495M, A519I, A520C, A520L, A520P, A535R, V536M, and A539R.

### Example 7

### Combined Specific Activity and Thermostability Variants

The variants in Examples 6-7 were analyzed fro bombined increased specific activity and increased thermal stability. Table 5 shows the variants with a PI>1.0 compared to the parent TrGA PI for both properties. These included the following sites: 10, 15, 59, 61, 68, 72, 73, 97, 99, 102, 140, 153, 182, 204, 205, 214, 228, 229, 230, 231, 236, 241, 242, 264, 265, 268, 276, 284, 291, 300, 301, 303, 311, 338, 344, 346, 349, 359, 361, 364, 375, 379, 382, 391, 393, 394, 410, 417, 430, 431, 433, 444, 448, 451, 495, 503, 511, 520, 531, 535, 536, and 539

TABLE 5
Combined variants

| | PI of | PI of |
|---|---|---|
| | Specific | Thermal |
| Variant | Activity | Stability |
| T010F | 1.06 | 1.11 |
| T010G | 1.12 | 1.13 |
| T010M | 1.05 | 1.12 |
| T010R | 1.08 | 1.06 |
| T010S | 1.09 | 1.24 |
| N015N | 1.06 | 1.06 |
| F059A | 1.17 | 1.03 |
| F059G | 1.05 | 1.07 |
| N061V | 1.11 | 1.06 |
| E068I | 1.06 | 1.19 |
| E068M | 1.10 | 1.29 |
| E068W | 1.03 | 1.09 |
| A072E | 1.11 | 1.07 |
| A072Q | 1.10 | 1.02 |
| G073W | 1.03 | 1.36 |
| S097V | 1.12 | 1.03 |
| A099C | 1.07 | 1.07 |
| A099L | 1.01 | 1.09 |
| S102A | 1.20 | 1.02 |
| K140Q | 1.08 | 1.06 |
| K140S | 1.13 | 1.05 |
| K140W | 1.07 | 1.04 |
| N153D | 1.20 | 1.06 |
| N153L | 1.12 | 1.06 |
| N153W | 1.07 | 1.19 |
| N182R | 1.03 | 1.02 |
| A204D | 1.09 | 1.02 |
| A204M | 1.09 | 1.08 |
| T205D | 1.03 | 1.06 |
| T205N | 1.09 | 1.09 |
| T205P | 1.17 | 1.17 |
| T205S | 1.10 | 1.04 |
| T205V | 1.06 | 1.06 |
| T205Y | 1.18 | 1.07 |
| S214P | 1.08 | 1.04 |
| S214T | 1.07 | 1.06 |
| W228A | 1.20 | 1.01 |
| W228F | 1.22 | 1.12 |
| W228G | 1.17 | 1.06 |
| W228H | 1.33 | 1.05 |
| W228I | 1.18 | 1.06 |
| W228L | 1.12 | 1.14 |
| W228M | 1.35 | 1.04 |
| W228T | 1.19 | 1.06 |
| V229E | 1.01 | 1.16 |
| V229I | 1.02 | 1.21 |
| V229M | 1.03 | 1.11 |
| V229N | 1.01 | 1.07 |
| V229Q | 1.02 | 1.10 |
| S230C | 1.23 | 1.09 |
| S230D | 1.13 | 1.08 |
| S230E | 1.10 | 1.11 |
| S230F | 1.63 | 1.08 |
| S230G | 1.77 | 1.09 |
| S230H | 1.05 | 1.03 |
| S230K | 1.04 | 1.02 |
| S230L | 1.20 | 1.02 |
| S230N | 1.23 | 1.16 |
| S230P | 1.13 | 1.12 |
| S230Q | 1.20 | 1.20 |
| S230R | 1.84 | 1.11 |
| S230T | 1.11 | 1.07 |
| S230V | 1.12 | 1.11 |
| S230Y | 1.08 | 1.05 |
| S231C | 1.13 | 1.07 |
| S231D | 1.08 | 1.18 |
| S231F | 1.17 | 1.14 |
| S231L | 1.29 | 1.15 |
| S231M | 1.08 | 1.09 |
| S231N | 1.04 | 1.13 |
| S231Q | 1.05 | 1.18 |
| S231R | 1.02 | 1.13 |
| S231V | 1.07 | 1.21 |
| S231Y | 1.07 | 1.10 |
| D236F | 1.14 | 1.11 |
| D236G | 1.05 | 1.07 |
| D236L | 1.11 | 1.08 |
| D236M | 1.07 | 1.15 |
| D236N | 1.03 | 1.15 |
| D236P | 1.06 | 1.06 |
| D236T | 1.14 | 1.16 |
| D236V | 1.04 | 1.18 |
| T241V | 1.05 | 1.05 |
| N242F | 1.06 | 1.03 |
| L264A | 1.04 | 1.03 |
| G265E | 1.10 | 1.03 |
| G265H | 1.12 | 1.09 |
| G265I | 1.06 | 1.09 |
| G265K | 1.03 | 1.05 |
| G265R | 1.06 | 1.12 |
| G265T | 1.10 | 1.17 |
| A268C | 1.50 | 1.16 |
| A268D | 1.14 | 1.20 |
| A268E | 1.18 | 1.10 |
| A268F | 1.15 | 1.07 |
| A268G | 1.35 | 1.11 |
| A268I | 1.15 | 1.12 |
| A268K | 1.23 | 1.11 |
| A268L | 1.06 | 1.11 |
| A268P | 1.08 | 1.13 |
| A268R | 1.14 | 1.15 |
| A268T | 1.18 | 1.14 |
| A268W | 1.05 | 1.05 |
| D276S | 1.01 | 1.02 |
| V284R | 1.06 | 1.09 |
| V284T | 1.05 | 1.08 |
| V284Y | 1.07 | 1.12 |
| S291A | 1.26 | 1.21 |
| S291E | 1.09 | 1.02 |
| S291F | 1.13 | 1.24 |
| S291H | 1.13 | 1.25 |
| S291K | 1.07 | 1.13 |
| S291N | 1.04 | 1.08 |
| S291P | 1.12 | 1.11 |
| S291W | 1.04 | 1.15 |
| P300R | 1.11 | 1.02 |
| A301K | 1.09 | 1.11 |
| A301L | 1.05 | 1.07 |
| A301P | 1.02 | 1.22 |
| A301S | 1.03 | 1.12 |
| A301W | 1.04 | 1.14 |
| A303I | 1.09 | 1.02 |
| A303L | 1.05 | 1.03 |
| A311S | 1.01 | 1.01 |
| A311Y | 1.06 | 1.02 |
| V338P | 1.04 | 1.18 |
| V338Q | 1.12 | 1.20 |
| V338S | 1.14 | 1.19 |
| V338Y | 1.05 | 1.10 |
| S344A | 1.05 | 1.05 |
| S344T | 1.01 | 1.19 |
| T346M | 1.06 | 1.07 |
| T346N | 1.09 | 1.15 |
| T346P | 1.07 | 1.13 |
| T346Q | 1.04 | 1.12 |
| T346Y | 1.06 | 1.07 |
| A349L | 1.02 | 1.16 |
| V359Q | 1.14 | 1.02 |
| V359R | 1.15 | 1.13 |
| G361H | 1.11 | 1.11 |
| G361L | 1.04 | 1.22 |
| G361R | 1.04 | 1.19 |
| A364M | 1.05 | 1.21 |
| A364W | 1.07 | 1.23 |
| T375E | 1.02 | 1.03 |
| T375H | 1.05 | 1.13 |
| T375V | 1.04 | 1.16 |
| T375W | 1.02 | 1.11 |
| T375Y | 1.05 | 1.17 |
| N379A | 1.05 | 1.11 |
| N379C | 1.10 | 1.03 |
| N379D | 1.05 | 1.06 |
| N379G | 1.07 | 1.09 |
| N379I | 1.01 | 1.04 |
| N379M | 1.04 | 1.08 |
| N379P | 1.06 | 1.17 |
| S382A | 1.02 | 1.06 |
| S382N | 1.02 | 1.07 |
| S382P | 1.10 | 1.10 |
| E391L | 1.18 | 1.04 |
| E391R | 1.08 | 1.02 |
| E391W | 1.12 | 1.04 |
| E391Y | 1.08 | 1.02 |
| A393E | 1.05 | 1.03 |
| A393G | 1.14 | 1.12 |
| A393H | 1.10 | 1.05 |
| A393I | 1.07 | 1.06 |
| A393K | 1.09 | 1.05 |
| A393L | 1.12 | 1.17 |
| A393M | 1.07 | 1.07 |
| A393N | 1.18 | 1.11 |
| A393Q | 1.02 | 1.02 |
| A393R | 1.09 | 1.03 |
| A393S | 1.10 | 1.13 |
| A393T | 1.13 | 1.08 |
| A393V | 1.16 | 1.09 |
| A393W | 1.04 | 1.10 |
| A393Y | 1.03 | 1.12 |
| K394A | 1.11 | 1.08 |
| K394H | 1.11 | 1.08 |
| K394L | 1.01 | 1.08 |
| K394M | 1.14 | 1.06 |
| K394Q | 1.09 | 1.07 |
| K394R | 1.19 | 1.06 |
| K394T | 1.08 | 1.03 |
| K394V | 1.05 | 1.02 |
| S410E | 1.01 | 1.05 |
| S410H | 1.06 | 1.11 |
| S410N | 1.04 | 1.10 |
| L417I | 1.10 | 1.04 |
| L417K | 1.02 | 1.20 |
| L417Q | 1.04 | 1.04 |
| L417R | 1.30 | 1.20 |
| L417V | 1.21 | 1.07 |
| L417Y | 1.10 | 1.01 |
| T430A | 1.19 | 1.05 |
| T430E | 1.15 | 1.02 |
| T430F | 1.09 | 1.06 |
| T430H | 1.15 | 1.10 |
| T430I | 1.06 | 1.04 |
| T430K | 1.24 | 1.08 |
| T430M | 1.16 | 1.17 |
| T430N | 1.07 | 1.13 |
| T430Q | 1.15 | 1.05 |
| T430R | 1.04 | 1.13 |
| T430V | 1.09 | 1.05 |
| A431I | 1.20 | 1.03 |
| A431R | 1.11 | 1.08 |
| R433A | 1.09 | 1.13 |
| R433M | 1.17 | 1.10 |
| R433W | 1.06 | 1.16 |
| R433Y | 1.22 | 1.18 |
| S444K | 1.03 | 1.07 |
| S444M | 1.13 | 1.05 |
| S444Q | 1.08 | 1.04 |
| T448F | 1.02 | 1.12 |
| T448I | 1.10 | 1.12 |
| T448Q | 1.04 | 1.17 |
| T448V | 1.04 | 1.13 |
| S451K | 1.29 | 1.08 |
| T495R | 1.15 | 1.10 |
| E503C | 1.05 | 1.39 |
| E503S | 1.10 | 1.02 |
| Q511H | 1.05 | 1.33 |
| A520C | 1.38 | 1.10 |
| A520L | 1.46 | 1.06 |
| A520P | 1.50 | 1.06 |
| V531L | 1.04 | 1.21 |
| A535K | 1.07 | 1.24 |
| A535N | 1.04 | 1.37 |
| A535P | 1.14 | 1.59 |
| A535R | 1.22 | 1.26 |
| V536I | 1.04 | 1.02 |
| A539E | 1.08 | 1.32 |
| A539M | 1.03 | 1.05 |
| A539R | 1.22 | 1.36 |
| A539S | 1.02 | 1.30 |

### Example 8

### Construction and characterization of combinatorial variants

Based on the data shown in Examples 5-7, a selected set of variants with single substitutions were further characterized. These variants have single substitution at positions: 43, 44, 61, 73, 294, 417, 430, 431, 503, 511, 535, 539, and 563. Among these sites, 43, 44, and 294 were identified in a previous screening experiment in Schizosaccharomyces pombe. See WO 08/045,489, which is incorporated herein by reference. Variants were purified from large-scale fermentation, and PIs of thermal stability and specific activities were determined. Specifically, specific activities were determined using various substrates, including DP7, cornstarch, and liquefact. The results are shown in Table 6.

TABLE 6
PIs of a selected set of single site variants, each of
which is obtained from a 500 ml fermentation.

| | P.I. DP7- | P.I. CornStarch- | P.I. Thermal | P.I. Liquefact- |
|---|---|---|---|---|
| Variants | FPLC | FPLC | Stability | FPLC |
| N61I | 1.16 | 1.35 | 1.00 | 1.66 |
| A431L | 1.15 | 1.38 | 1.18 | 1.51 |
| L417V | 1.18 | 1.32 | 1.02 | 1.40 |
| A431Q | 1.06 | 1.20 | 0.92 | 1.24 |
| G294C | 1.01 | 0.84 | 0.94 | 1.23 |
| N563K | 1.07 | 1.12 | 1.97 | 1.15 |
| Q511H | 1.05 | 1.09 | 1.52 | 1.13 |
| T430M | 1.05 | 1.15 | 0.89 | 1.09 |
| E503A | 1.08 | 1.16 | 1.40 | 1.09 |
| I43Q | 1.11 | 1.24 | 0.94 | 1.08 |
| A539R | 1.15 | 1.37 | 1.43 | 1.08 |
| I43R | 1.03 | 1.07 | 1.41 | 1.07 |
| L417R | 1.23 | 1.27 | 1.51 | 1.04 |
| T430A | 1.13 | 1.35 | 1.23 | 1.04 |
| G73F | 1.06 | 1.06 | 1.45 | 1.03 |
| D44R | 0.97 | 1.06 | 1.46 | 0.98 |
| N563I | 1.09 | 1.22 | 2.06 | 0.92 |
| D44C | 0.80 | 0.82 | 0.96 | 0.91 |
| E503V | 1.17 | 1.07 | 1.66 | 0.88 |
| A535R | 1.09 | 1.44 | 1.47 | 0.85 |

Additionally, combinatorial variants were constructed using the PCR method with substitutions among: 43, 44, 61, 73, 294, 417, 430, 431, 503, 511, 535, 539, and 563. Briefly, the combinatorial variants were constructed by using plasmid pDONR-TrGA (FIG. 2) as the backbone. The methodology to construct combinatorial variants is based on the Gateway technology (Invitrogen, Carlsbad, Calif.). The primers used to create the combinatorial variants are shown in Tables 2 and 7. The following synthetic construct approach was chosen for the construction of all combinatorial variants.

CTCTCT [XbaI site] [MF] GAGAGGGG [attB1] [GAP combinatorial variant] [attB2 sites] CCCCAGAG [MR] [HindIII] AGAGAG
This construct was treated with restriction enzymes Xba-I and HindIII. The digested fragments were ligated into Xba-1/HindIII treated pBC (a pUC19 derived vector). The ligation mixture was transformed to E. coli DH10B (Invitrogen, Carlsbad, Calif.) and plated onto selective agar supplemented with 100 vg/ml ampicillin. The plates were incubated for 16 h at 37° C. Colonies from the selective plates were isolated and inoculated into selective liquid medium. After 16 h incubation at 37° C. and 250 rpm the plasmids were isolated using a standard plasmid isolation kit and combined with pDONR 2.21 (Invitrogen, Carlsbad, Calif.) to create a Gateway entry vector with the specific GAP combinatorial variants. The reaction mixture was transformed into E. coli Max efficiency DH5a (Invitrogen, Carlsbad, Calif.) and plated on selective agar (2×TY supplemented with 50 μg kanamycin/ml). After overnight incubation at 37° C., single colonies were picked for sequence analysis (BaseClear B.V., Leiden, Netherlands). The combinatorial variants were subcloned in pTrexTrTel and expressed in a T. reesei host strain as described in WO 06/060062.

TABLE 7
Primers used to construct combinatorial variants

| | | SEQ ID |
|---|---|---|
| Primer | DNA sequence | NO: |
| Var1 Basic-1 | GAGAGAGTGCGGGCCTCTTCGCTATTTCTAGA | 391 |
| Var1 Basic-2 | CAAAATAAAATCATTATTTGTCTAGAAATAGCGAAGAGGC | 392 |
| Var1 Basic-3 | CAAATAATGATTTTATTTTGACTGATAGTGACCTGTTCGT | 393 |
| Var1 Basic-4 | TTGCTCATCAATGTGTTGCAACGAACAGGTCACTATCAGT | 394 |
| Var1 Basic-5 | TGCAACACATTGATGAGCAATGCTTTTTTATAATGCCAAC | 395 |
| Var1 Basic-6 | AGCCTGCTTTTTTGTACAAAGTTGGCATTATAAAAAAGCA | 396 |
| Var1 Basic-7 | TTTGTACAAAAAAGCAGGCTATGCACGTCCTGTCGACTGC | 397 |
| Var1 Basic-8 | CAACGGAGCCGAGCAGCACCGCAGTCGACAGGACGTGCAT | 398 |
| Var1 Basic-9 | GGTGCTGCTCGGCTCCGTTGCCGTTCAAAAGGTCCTGGGA | 399 |
| Var1 Basic-10 | AGACCGCTTGATCCTGGTCTTCCCAGGACCTTTTGAACGG | 400 |
| Var1 Basic-11 | AGACCAGGATCAAGCGGTCTGTCCGACGTCACCAAGAGGT | 401 |
| Var1 Basic-12 | GCTGATGAAGTCGTCAACAGACCTCTTGGTGACGTCGGAC | 402 |
| Var1 Basic-13 | CTGTTGACGACTTCATCAGCACCGAGACGCCTATTGCACT | 403 |
| Var1 Basic-14 | CATTGCAAAGAAGATTGTTCAGTGCAATAGGCGTCTCGGT | 404 |
| Var1 Basic-15 | GAACAATCTTCTTTGCAATGTTGGTCCTGATGGATGCCGT | 405 |
| Var1 Basic-16 | CCAGCTGATGTGCCGAATGCACGGCATCCATCAGGACCAA | 406 |
| Var1 Basic-17 | GCATTCGGCACATCAGCTGGTGCGGTGATTGCATCTCCCA | 407 |
| Var1 Basic-18 | GTAGTCCGGGTCAATTGTGCTGGGAGATGCAATCACCGCA | 408 |
| Var1 Basic-19 | GCACAATTGACCCGGACTACTATTACATGTGGACGCGAGA | 409 |
| Var1 Basic-20 | TCTTGAAGACAAGAGCGCTATCTCGCGTCCACATGTAATA | 410 |
| Var1 Basic-21 | TAGCGCTCTTGTCTTCAAGAACCTCATCGACCGCTTCACC | 411 |
| Var1 Basic-22 | AGGCCCGCATCGTACGTTTCGGTGAAGCGGTCGATGAGGT | 412 |
| Var1 Basic-23 | GAAACGTACGATGCGGGCCTGCAGCGCCGCATCGAGCAGT | 413 |
| Var1 Basic-24 | AGTGACCTGGGCAGTAATGTACTGCTCGATGCGGCGCTGC | 414 |
| Var1 Basic-25 | ACATTACTGCCCAGGTCACTCTCCAGGGCCTCTCTAACCC | 415 |
| Var1 Basic-26 | CGTCCGCGAGGGAGCCCGAGGGGTTAGAGAGGCCCTGGAG | 416 |
| Var1 Basic-27 | CTCGGGCTCCCTCGCGGACGGCTCTGGTCTCGGCGAGCCC | 417 |
| Var1 Basic-28 | TTCAGGGTCAACTCAAACTTGGGCTCGCCGAGACCAGAGC | 418 |
| Var1 Basic-29 | AAGTTTGAGTTGACCCTGAAGCCTTTCACCGGCAACTGGG | 419 |
| Var1 Basic-30 | GCCATCCCGCTGCGGTCGACCCCAGTTGCCGGTGAAAGGC | 420 |
| Var1 Basic-31 | GTCGACCGCAGCGGGATGGCCCAGCTCTGCGAGCCATTGC | 421 |
| Var1 Basic-32 | ACTTTGAGTATCCAATCAAGGCAATGGCTCGCAGAGCTGG | 422 |
| Var1 Basic-33 | CTTGATTGGATACTCAAAGTGGCTCATCAACAACAACTAT | 423 |
| Var1 Basic-34 | ACGTTGGACACAGTCGACTGATAGTTGTTGTTGATGAGCC | 424 |
| Var1 Basic-35 | CAGTCGACTGTGTCCAACGTCATCTGGCCTATTGTGCGCA | 425 |
| Var1 Basic-36 | GGCAACATAGTTGAGGTCGTTGCGCACAATAGGCCAGATG | 426 |
| Var1 Basic-37 | ACGACCTCAACTATGTTGCCCAGTACTGGAACCAAACCGG | 427 |
| Var1 Basic-38 | CTTCTTCCCAGAGGTCAAAGCCGGTTTGGTTCCAGTACTG | 428 |
| Var1 Basic-39 | CTTTGACCTCTGGGAAGAAGTCAATGGGAGCTCATTCTTT | 429 |
| Var1 Basic-40 | CGGTGCTGGTTGGCAACAGTAAAGAATGAGCTCCCATTGA | 430 |
| Var1 Basic-41 | ACTGTTGCCAACCAGCACCGAGCACTTGTCGAGGGCGCCA | 431 |
| Var1 Basic-42 | GCCAAGAGTGGCAGCAAGAGTGGCGCCCTCGACAAGTGCT | 432 |
| Var1 Basic-43 | CTCTTGCTGCCACTCTTGGCCAGTCGGGAAGCGCTTATTC | 433 |
| Var1 Basic-44 | AAACCTGGGGAGCAACAGATGAATAAGCGCTTCCCGACTG | 434 |
| Var1 Basic-45 | ATCTGTTGCTCCCCAGGTTTTGTGCTTTCTCCAACGATTC | 435 |
| Var1 Basic-46 | TATCCACCAGACGACACCCAGAATCGTTGGAGAAAGCACA | 436 |
| Var1 Basic-47 | TGGGTGTCGTCTGGTGGATACGTCGACTCCAACATCAACA | 437 |
| Var1 Basic-48 | GCCAGTCCTGCCCTCGTTGGTGTTGATGTTGGAGTCGACG | 438 |
| Var1 Basic-49 | CCAACGAGGGCAGGACTGGCAAGGATGTCAACTCCGTCCT | 439 |
| Var1 Basic-50 | CGAAGGTGTGGATGGAAGTCAGGACGGAGTTGACATCCTT | 440 |
| Var1 Basic-51 | GACTTCCATCCACACCTTCGATCCCAACCTTGGCTGTGAC | 441 |
| Var1 Basic-52 | CATGGCTGGAAGGTGCCTGCGTCACAGCCAAGGTTGGGAT | 442 |
| Var1 Basic-53 | GCAGGCACCTTCCAGCCATGCAGTGACAAAGCGCTCTCCA | 443 |
| Var1 Basic-54 | GTCGACAACAACCTTGAGGTTGGAGAGCGCTTTGTCACTG | 444 |
| Var1 Basic-55 | ACCTCAAGGTTGTTGTCGACTCCTTCCGCTCCATCTACGG | 445 |
| Var1 Basic-56 | CAGGAATGCCCTTGTTCACGCCGTAGATGGAGCGGAAGGA | 446 |
| Var1 Basic-57 | CGTGAACAAGGGCATTCCTGCCGGTGCTGCCGTCGCCATT | 447 |
| Var1 Basic-58 | ACATCCTCTGCATACCGGCCAATGGCGACGGCAGCACCGG | 448 |
| Var1 Basic-59 | GGCCGGTATGCAGAGGATGTGTACTACAACGGCAACCCTT | 449 |
| Var1 Basic-60 | AGCAAATGTAGCAAGATACCAAGGGTTGCCGTTGTAGTAC | 450 |
| Var1 Basic-61 | GGTATCTTGCTACATTTGCTGCTGCCGAGCAGCTGTACGA | 451 |
| Var1 Basic-62 | TCTTCCAGACGTAGATGGCATCGTACAGCTGCTCGGCAGC | 452 |
| Var1 Basic-63 | TGCCATCTACGTCTGGAAGAAGACGGGCTCCATCACGGTG | 453 |
| Var1 Basic-64 | AAGGCCAGGGAGGTGGCGGTCACCGTGATGGAGCCCGTCT | 454 |
| Var1 Basic-65 | ACCGCCACCTCCCTGGCCTTCTTCCAGGAGCTTGTTCCTG | 455 |
| Var1 Basic-66 | GTAGGTCCCGGCCGTCACGCCAGGAACAAGCTCCTGGAAG | 456 |
| Var1 Basic-67 | GCGTGACGGCCGGGACCTACTCCAGCAGCTCTTCGACCTT | 457 |
| Var1 Basic-68 | CGGCGTTGATGATGTTGGTAAAGGTCGAAGAGCTGCTGGA | 458 |
| Var1 Basic-69 | TACCAACATCATCAACGCCGTCTCGACATACGCCGATGGC | 459 |
| Var1 Basic-70 | TTGGCAGCCTCGCTGAGGAAGCCATCGGCGTATGTCGAGA | 460 |
| Var1 Basic-71 | TTCCTCAGCGAGGCTGCCAAGTACGTCCCCGCCGACGGTT | 461 |
| Var1 Basic-72 | GTCAAACTGCTCGGCCAGCGAACCGTCGGCGGGGACGTAC | 462 |
| Var1 Basic-73 | CGCTGGCCGAGCAGTTTGACCGCAACAGCGGCACTCCGCT | 463 |
| Var1 Basic-74 | ACGTCAGGTGAACCGCAGACAGCGGAGTGCCGCTGTTGCG | 464 |
| Var1 Basic-75 | GTCTGCGGTTCACCTGACGTGGTCGTACGCCTCGTTCTTG | 465 |
| Var1 Basic-76 | GCCCGACGAAGCGTGGCTGTCAAGAACGAGGCGTACGACC | 466 |
| Var1 Basic-77 | ACAGCCACGCTTCGTCGGGCTGGCATCGTGCCCCCCTCGT | 467 |
| Var1 Basic-78 | GCTAGCGCTGCTGTTGGCCCACGAGGGGGGCACGATGCCA | 468 |
| Var1 Basic-79 | GGGCCAACAGCAGCGCTAGCACGATCCCCTCGACGTGCTC | 469 |
| Var1 Basic-80 | ATCCGACCACGGACGCGCCGGAGCACGTCGAGGGGATCGT | 470 |
| Var1 Basic-81 | CGGCGCGTCCGTGGTCGGATCCTACTCGCGTCCCACCGCC | 471 |
| Var1 Basic-82 | TGCGACGGAGGGAATGACGTGGCGGTGGGACGCGAGTAGG | 472 |
| Var1 Basic-83 | ACGTCATTCCCTCCGTCGCAGACGCCCAAGCCTGGCGTGC | 473 |
| Var1 Basic-84 | CGTGTAGGGAGTACCGGAAGGCACGCCAGGCTTGGGCGTC | 474 |
| Var1 Basic-85 | CTTCCGGTACTCCCTACACGCCCCTGCCCTGCGCGACCCC | 475 |
| Var1 Basic-86 | AGGTGACGGCCACGGAGGTTGGGGTCGCGCAGGGCAGGGG | 476 |
| Var1 Basic-87 | AACCTCCGTGGCCGTCACCTTCCACGAGCTCGTGTCGACA | 477 |
| Var1 Basic-88 | TTGACCGTCTGGCCAAACTGTGTCGACACGAGCTCGTGGA | 478 |
| Var1 Basic-89 | CAGTTTGGCCAGACGGTCAAGGTGGCGGGCAACGCCGCGG | 479 |
| Var1 Basic-90 | CGTGCTCCAGTTGCCCAGGGCCGCGGCGTTGCCCGCCACC | 480 |
| Var1 Basic-91 | CCCTGGGCAACTGGAGCACGAGCGCCGCCGTGGCTCTGGA | 481 |
| Var1 Basic-92 | TATCACGATAGTTGACGGCGTCCAGAGCCACGGCGGCGCT | 482 |
| Var1 Basic-93 | CGCCGTCAACTATCGTGATAACCACCCCCTGTGGATTGGG | 483 |
| Var1 Basic-94 | CCAGCCTCGAGGTTGACCGTCCCAATCCACAGGGGGTGGT | 484 |
| Var1 Basic-95 | ACGGTCAACCTCGAGGCTGGAGACGTCGTGGAGTACAAGT | 485 |
| Var1 Basic-96 | ATCTTGGCCCACATTGATGTACTTGTACTCCACGACGTCT | 486 |
| Var1 Basic-97 | ACATCAATGTGGGCCAAGATGGCTCCGTGACCTGGGAGAG | 487 |
| Var1 Basic-98 | TGTAAGTGTGGTTGGGATCACTCTCCCAGGTCACGGAGCC | 488 |
| Var1 Basic-99 | TGATCCCAACCACACTTACACGGTTCCTGCGGTGGCTTGT | 489 |
| Var1 Basic-100 | TCCTTGACAACCTGCGTCACACAAGCCACCGCAGGAACCG | 490 |
| Var1 Basic-101 | GTGACGCAGGTTGTCAAGGAGGACACCTGGCAGTCGTAAA | 491 |
| Var1 Basic-102 | CTTTGTACAAGAAAGCTGGGTTTACGACTGCCAGGTGTCC | 492 |
| Var1 Basic-103 | CCCAGCTTTCTTGTACAAAGTTGGCATTATAAGAAAGCAT | 493 |
| Var1 Basic-104 | TTGCAACAAATTGATAAGCAATGCTTTCTTATAATGCCAA | 494 |
| Var1 Basic-105 | TGCTTATCAATTTGTTGCAACGAACAGGTCACTATCAGTC | 495 |
| Var1 Basic-106 | TCAAATAATGATTTTATTTTGACTGATAGTGACCTGTTCG | 496 |
| Var1 Basic-107 | AAAATAAAATCATTATTTGAAGCTTAAGCCTGGGGTGCCT | 497 |
| Var1 Basic-108 | AGAGAGTCATTAGGCACCCCAGGCTTAAGCT | 498 |
| Var2 -18 | GTAGTCCGGGTCTTGTGTGCTGGGAGATGCAATCACCGCA | 499 |
| Var2 -19 | GCACACAAGACCCGGACTACTATTACATGTGGACGCGAGA | 500 |
| Var3 -21 | TAGCGCTCTTGTCTTCAAGATTCTCATCGACCGCTTCACC | 501 |
| Var3 -22 | AGGCCCGCATCGTACGTTTCGGTGAAGCGGTCGATGAGAA | 502 |
| Var4 -92 | TATCACGATAGTTGACACGGTCCAGAGCCACGGCGGCGCT | 503 |
| Var4 -93 | CCGTGTCAACTATCGTGATAACCACCCCCTGTGGATTGGG | 504 |
| Var5 -18 | GTAGTCCGGGTCTTGTGTGCTGGGAGATGCAATCACCGCA | 505 |
| Var5 -19 | GCACACAAGACCCGGACTACTATTACATGTGGACGCGAGA | 506 |
| Var5 -21 | TAGCGCTCTTGTCTTCAAGATTCTCATCGACCGCTTCACC | 507 |
| Var5 -22 | AGGCCCGCATCGTACGTTTCGGTGAAGCGGTCGATGAGAA | 508 |
| Var6 -21 | TAGCGCTCTTGTCTTCAAGATTCTCATCGACCGCTTCACC | 509 |
| Var6 -22 | AGGCCCGCATCGTACGTTTCGGTGAAGCGGTCGATGAGAA | 510 |
| Var6 -92 | TATCACGATAGTTGACACGGTCCAGAGCCACGGCGGCGCT | 511 |
| Var6 -93 | CCGTGTCAACTATCGTGATAACCACCCCCTGTGGATTGGG | 512 |
| Var7 -92 | TATCACGATAGTTGACACGGTCCAGAGCCACGGCGGCGCT | 513 |
| Var7 -93 | CCGTGTCAACTATCGTGATAACCACCCCCTGTGGATTGGG | 514 |
| Var7 -18 | GTAGTCCGGGTCTTGTGTGCTGGGAGATGCAATCACCGCA | 515 |
| Var7 -19 | GCACACAAGACCCGGACTACTATTACATGTGGACGCGAGA | 516 |
| Var8 -18 | GTAGTCCGGGTCTTGTGTGCTGGGAGATGCAATCACCGCA | 517 |
| Var8 -19 | GCACACAAGACCCGGACTACTATTACATGTGGACGCGAGA | 518 |
| Var8 -21 | TAGCGCTCTTGTCTTCAAGATTCTCATCGACCGCTTCACC | 519 |
| Var8 -22 | AGGCCCGCATCGTACGTTTCGGTGAAGCGGTCGATGAGAA | 520 |
| Var8 -92 | TATCACGATAGTTGACACGGTCCAGAGCCACGGCGGCGCT | 521 |
| Var8 -93 | CCGTGTCAACTATCGTGATAACCACCCCCTGTGGATTGGG | 522 |
| Var9 -18 | GTAGTCCGGGTCTTGTGTGCTGGGAGATGCAATCACCGCA | 523 |
| Var9 -19 | GCACACAAGACCCGGACTACTATTACATGTGGACGCGAGA | 524 |
| Var9 -21 | TAGCGCTCTTGTCTTCAAGATTCTCATCGACCGCTTCACC | 525 |
| Var9 -22 | AGGCCCGCATCGTACGTTTCGGTGAAGCGGTCGATGAGAA | 526 |
| Var9 -92 | TATCACGATAGTTGACACGGTCCAGAGCCACGGCGGCGCT | 527 |
| Var9 -93 | CCGTGTCAACTATCGTGATAACCACCCCCTGTGGATTGGG | 528 |
| Var9 -76 | GCCCGACGAAGAGCGGCTGTCAAGAACGAGGCGTACGACC | 529 |
| Var9 -77 | ACAGCCGCTCTTCGTCGGGCTGGCATCGTGCCCCCCTCGT | 530 |
| Var10 -76 | GCCCGACGAAGAGCGGCTGTCAAGAACGAGGCGTACGACC | 531 |
| Var10 -77 | ACAGCCGCTCTTCGTCGGGCTGGCATCGTGCCCCCCTCGT | 532 |
| Var10 -88 | TTGACCGTATGGCCAAACTGTGTCGACACGAGCTCGTGGA | 533 |
| Var10 -89 | CAGTTTGGCCATACGGTCAAGGTGGCGGGCAACGCCGCGG | 534 |
| Var10 -92 | TATCACGATAGTTGACACGGTCCAGAGCCACGGCGGCGCT | 535 |
| Var10 -93 | CCGTGTCAACTATCGTGATAACCACCCCCTGTGGATTGGG | 536 |
| Var10 -96 | ATCTTGGCCCACAATGATGTACTTGTACTCCACGACGTCT | 537 |
| Var10 -97 | ACATCATTGTGGGCCAAGATGGCTCCGTGACCTGGGAGAG | 538 |
| Var10 -18 | GTAGTCCGGGTCTTGTGTGCTGGGAGATGCAATCACCGCA | 539 |
| Var10 -19 | GCACACAAGACCCGGACTACTATTACATGTGGACGCGAGA | 540 |
| Var11 -76 | GCCCGACGAAGAGCGGCTGTCAAGAACGAGGCGTACGACC | 541 |
| Var11 -77 | ACAGCCGCTCTTCGTCGGGCTGGCATCGTGCCCCCCTCGT | 542 |
| Var11 -88 | TTGACCGTATGGCCAAACTGTGTCGACACGAGCTCGTGGA | 543 |
| Var11 -89 | CAGTTTGGCCATACGGTCAAGGTGGCGGGCAACGCCGCGG | 544 |
| Var11 -92 | TATCACGATAGTTGACACGGTCCAGAGCCACGGCGGCGCT | 545 |
| Var11 -93 | CCGTGTCAACTATCGTGATAACCACCCCCTGTGGATTGGG | 546 |
| Var11 -96 | ATCTTGGCCCACAATGATGTACTTGTACTCCACGACGTCT | 547 |
| Var11 -97 | ACATCATTGTGGGCCAAGATGGCTCCGTGACCTGGGAGAG | 548 |
| Var11 -21 | TAGCGCTCTTGTCTTCAAGATTCTCATCGACCGCTTCACC | 549 |
| Var11 -22 | AGGCCCGCATCGTACGTTTCGGTGAAGCGGTCGATGAGAA | 550 |
| Var12 -76 | GCCCGACGAAGAGCGGCTGTCAAGAACGAGGCGTACGACC | 551 |
| Var12 -77 | ACAGCCGCTCTTCGTCGGGCTGGCATCGTGCCCCCCTCGT | 552 |
| Var12 -88 | TTGACCGTATGGCCAAACTGTGTCGACACGAGCTCGTGGA | 553 |
| Var12 -89 | CAGTTTGGCCATACGGTCAAGGTGGCGGGCAACGCCGCGG | 554 |
| Var12 -92 | TATCACGATAGTTGACACGGTCCAGAGCCACGGCGGCGCT | 555 |
| Var12 -93 | CCGTGTCAACTATCGTGATAACCACCCCCTGTGGATTGGG | 556 |
| Var12 -96 | ATCTTGGCCCACAATGATGTACTTGTACTCCACGACGTCT | 557 |
| Var12 -97 | ACATCATTGTGGGCCAAGATGGCTCCGTGACCTGGGAGAG | 558 |
| Var12 -18 | GTAGTCCGGGTCTTGTGTGCTGGGAGATGCAATCACCGCA | 559 |
| Var12 -19 | GCACACAAGACCCGGACTACTATTACATGTGGACGCGAGA | 560 |
| Var12 -21 | TAGCGCTCTTGTCTTCAAGATTCTCATCGACCGCTTCACC | 561 |
| Var12 -22 | AGGCCCGCATCGTACGTTTCGGTGAAGCGGTCGATGAGAA | 562 |
| Var13 -18 | GTAGTCCGGGTCACGTGTGCTGGGAGATGCAATCACCGCA | 563 |
| Var13 -19 | GCACACGTGACCCGGACTACTATTACATGTGGACGCGAGA | 564 |
| Var14 -18 | GTAGTCCGGGTCACGTGTGCTGGGAGATGCAATCACCGCA | 565 |
| Var14 -19 | GCACACGTGACCCGGACTACTATTACATGTGGACGCGAGA | 566 |
| Var14 -21 | TAGCGCTCTTGTCTTCAAGATTCTCATCGACCGCTTCACC | 567 |
| Var14 -22 | AGGCCCGCATCGTACGTTTCGGTGAAGCGGTCGATGAGAA | 568 |
| Var15 -18 | GTAGTCCGGGTCACGTGTGCTGGGAGATGCAATCACCGCA | 569 |
| Var15 -19 | GCACACGTGACCCGGACTACTATTACATGTGGACGCGAGA | 570 |
| Var15 -21 | TAGCGCTCTTGTCTTCAAGATTCTCATCGACCGCTTCACC | 571 |
| Var15 -22 | AGGCCCGCATCGTACGTTTCGGTGAAGCGGTCGATGAGAA | 572 |
| Var15 -92 | TATCACGATAGTTGACACGGTCCAGAGCCACGGCGGCGCT | 573 |
| Var15 -93 | CCGTGTCAACTATCGTGATAACCACCCCCTGTGGATTGGG | 574 |
| Var15 -76 | GCCCGACGAAGAGCGGCTGTCAAGAACGAGGCGTACGACC | 575 |
| Var15 -77 | ACAGCCGCTCTTCGTCGGGCTGGCATCGTGCCCCCCTCGT | 576 |
| Var16 -74 | ACGTCAGGTGACGCGCAGACAGCGGAGTGCCGCTGTTGCG | 577 |
| Var16 -75 | GTCTGCGCGTCACCTGACGTGGTCGTACGCCTCGTTCTTG | 578 |
| Var17 -74 | ACGTCAGGTGACCCGCAGACAGCGGAGTGCCGCTGTTGCG | 579 |
| Var17 -75 | GTCTGCGGGTCACCTGACGTGGTCGTACGCCTCGTTCTTG | 580 |
| Var18 -22 | AGAAACGCATCGTACGTTTCGGTGAAGCGGTCGATGAGGT | 581 |
| Var18 -23 | GAAACGTACGATGCGTTTCTGCAGCGCCGCATCGAGCAGT | 582 |
| Var18 -74 | ACGTCAGGTGACGCGCAGACAGCGGAGTGCCGCTGTTGCG | 583 |
| Var18 -75 | GTCTGCGCGTCACCTGACGTGGTCGTACGCCTCGTTCTTG | 584 |
| Var18 -76 | GCCCGACGGGCCGTGGCTGTCAAGAACGAGGCGTACGACC | 585 |
| Var18 -77 | ACAGCCACGGCCCGTCGGGCTGGCATCGTGCCCCCCTCGT | 586 |
| Var18 -87 | AACCTCCGTGGCCGTCACCTTCCACGTTCTCGTGTCGACA | 587 |
| Var18 -88 | TTGACCGTCTGGCCAAACTGTGTCGACACGAGAACGTGGA | 588 |
| Var18 -96 | ATCTTGGCCCACTTTGATGTACTTGTACTCCACGACGTCT | 589 |
| Var18 -97 | ACATCAAAGTGGGCCAAGATGGCTCCGTGACCTGGGAGAG | 590 |
| Var19 -18 | GTAGTCCGGGTCACGTGTGCTGGGAGATGCAATCACCGCA | 591 |
| Var19 -19 | GCACACGTGACCCGGACTACTATTACATGTGGACGCGAGA | 592 |
| Var19 -22 | AGAAACGCATCGTACGTTTCGGTGAAGCGGTCGATGAGGT | 593 |
| Var19 -23 | GAAACGTACGATGCGTTTCTGCAGCGCCGCATCGAGCAGT | 594 |
| Var19 -74 | ACGTCAGGTGACGCGCAGACAGCGGAGTGCCGCTGTTGCG | 595 |
| Var19 -75 | GTCTGCGCGTCACCTGACGTGGTCGTACGCCTCGTTCTTG | 596 |
| Var19 -76 | GCCCGACGGGCCGTGGCTGTCAAGAACGAGGCGTACGACC | 597 |
| Var19 -77 | ACAGCCACGGCCCGTCGGGCTGGCATCGTGCCCCCCTCGT | 598 |
| Var19 -87 | AACCTCCGTGGCCGTCACCTTCCACGTTCTCGTGTCGACA | 599 |
| Var19 -88 | TTGACCGTCTGGCCAAACTGTGTCGACACGAGAACGTGGA | 600 |
| Var19 -96 | ATCTTGGCCCACTTTGATGTACTTGTACTCCACGACGTCT | 601 |
| Var19 -97 | ACATCAAAGTGGGCCAAGATGGCTCCGTGACCTGGGAGAG | 602 |
| Var20 -18 | GTAGTCCGGGTCACGTGTGCTGGGAGATGCAATCACCGCA | 603 |
| Var20 -19 | GCACACGTGACCCGGACTACTATTACATGTGGACGCGAGA | 604 |
| Var20 -22 | AGAAACGCATCGTACGTTTCGGTGAAGCGGTCGATGAGGT | 605 |
| Var20 -23 | GAAACGTACGATGCGTTTCTGCAGCGCCGCATCGAGCAGT | 606 |
| Var20 -74 | ACGTCAGGTGAAGCGCAGACAGCGGAGTGCCGCTGTTGCG | 607 |
| Var20 -75 | GTCTGCGCTTCACCTGACGTGGTCGTACGCCTCGTTCTTG | 608 |
| Var20 -76 | GCCCGACGGGCCGTGGCTGTCAAGAACGAGGCGTACGACC | 609 |
| Var20 -77 | ACAGCCACGGCCCGTCGGGCTGGCATCGTGCCCCCCTCGT | 610 |
| Var20 -87 | AACCTCCGTGGCCGTCACCTTCCACGTTCTCGTGTCGACA | 611 |
| Var20 -88 | TTGACCGTATGGCCAAACTGTGTCGACACGAGAACGTGGA | 612 |
| Var20 -89 | CAGTTTGGCCATACGGTCAAGGTGGCGGGCAACGCCGCGG | 613 |
| Var20 -93 | TATCGGCATAGTTGACGGCGTCCAGAGCCACGGCGGCGCT | 614 |
| Var20 -94 | CGCCGTCAACTATGCCGATAACCACCCCCTGTGGATTGGG | 615 |
| Var20 -96 | ATCTTGGCCCACTTTGATGTACTTGTACTCCACGACGTCT | 616 |
| Var20 -97 | ACATCAAAGTGGGCCAAGATGGCTCCGTGACCTGGGAGAG | 617 |
| GAV Basic-1 | ACAAGTTTGTACAAAAAAGCAGGCT | 618 |
| GAV Basic-2 | GCAGTCGACAGGACGTGCATAGCCTGCTTTTTTGTACAAA | 619 |
| GAV Basic-3 | ATGCACGTCCTGTCGACTGCGGTGCTGCTCGGCTCCGTTG | 620 |
| GAV Basic-4 | TCCCAGGACCTTTTGAACGGCAACGGAGCCGAGCAGCACC | 621 |
| GAV Basic-5 | CCGTTCAAAAGGTCCTGGGAAGACCAGGATCAAGCGGTCT | 622 |
| GAV Basic-6 | ACCTCTTGGTGACGTCGGACAGACCGCTTGATCCTGGTCT | 623 |
| GAV Basic-7 | GTCCGACGTCACCAAGAGGTCTGTTGACGACTTCATCAGC | 624 |
| GAV Basic-8 | AGTGCAATAGGCGTCTCGGTGCTGATGAAGTCGTCAACAG | 625 |
| GAV Basic-9 | ACCGAGACGCCTATTGCACTGAACAATCTTCTTTGCAATG | 626 |
| GAV Basic-10 | ACGGCATCCATCAGGACCAACATTGCAAAGAAGATTGTTC | 627 |
| GAV Basic-11 | TTGGTCCTGATGGATGCCGTGCATTCGGCACATCAGCTGG | 628 |
| GAV Basic-12 | TGGGAGATGCAATCACCGCACCAGCTGATGTGCCGAATGC | 629 |
| GAV Basic-13 | TGCGGTGATTGCATCTCCCAGCACAATTGACCCGGACTAC | 630 |
| GAV Basic-14 | TCTCGCGTCCACATGTAATAGTAGTCCGGGTCAATTGTGC | 631 |
| GAV Basic-15 | TATTACATGTGGACGCGAGATAGCGCTCTTGTCTTCAAGA | 632 |
| GAV Basic-16 | GGTGAAGCGGTCGATGAGGTTCTTGAAGACAAGAGCGCTA | 633 |
| GAV Basic-17 | ACCTCATCGACCGCTTCACCGAAACGTACGATGCGGGCCT | 634 |
| GAV Basic-18 | ACTGCTCGATGCGGCGCTGCAGGCCCGCATCGTACGTTTC | 635 |
| GAV Basic-19 | GCAGCGCCGCATCGAGCAGTACATTACTGCCCAGGTCACT | 636 |
| GAV Basic-20 | GGGTTAGAGAGGCCCTGGAGAGTGACCTGGGCAGTAATGT | 637 |
| GAV Basic-21 | CTCCAGGGCCTCTCTAACCCCTCGGGCTCCCTCGCGGACG | 638 |
| GAV Basic-22 | GGGCTCGCCGAGACCAGAGCCGTCCGCGAGGGAGCCCGAG | 639 |
| GAV Basic-23 | GCTCTGGTCTCGGCGAGCCCAAGTTTGAGTTGACCCTGAA | 640 |
| GAV Basic-24 | CCCAGTTGCCGGTGAAAGGCTTCAGGGTCAACTCAAACTT | 641 |
| GAV Basic-25 | GCCTTTCACCGGCAACTGGGGTCGACCGCAGCGGGATGGC | 642 |
| GAV Basic-26 | GCAATGGCTCGCAGAGCTGGGCCATCCCGCTGCGGTCGAC | 643 |
| GAV Basic-27 | CCAGCTCTGCGAGCCATTGCCTTGATTGGATACTCAAAGT | 644 |
| GAV Basic-28 | ATAGTTGTTGTTGATGAGCCACTTTGAGTATCCAATCAAG | 645 |
| GAV Basic-29 | GGCTCATCAACAACAACTATCAGTCGACTGTGTCCAACGT | 646 |
| GAV Basic-30 | TGCGCACAATAGGCCAGATGACGTTGGACACAGTCGACTG | 647 |
| GAV Basic-31 | CATCTGGCCTATTGTGCGCAACGACCTCAACTATGTTGCC | 648 |
| GAV Basic-32 | CCGGTTTGGTTCCAGTACTGGGCAACATAGTTGAGGTCGT | 649 |
| GAV Basic-33 | CAGTACTGGAACCAAACCGGCTTTGACCTCTGGGAAGAAG | 650 |
| GAV Basic-34 | AAAGAATGAGCTCCCATTGACTTCTTCCCAGAGGTCAAAG | 651 |
| GAV Basic-35 | TCAATGGGAGCTCATTCTTTACTGTTGCCAACCAGCACCG | 652 |
| GAV Basic-36 | TGGCGCCCTCGACAAGTGCTCGGTGCTGGTTGGCAACAGT | 653 |
| GAV Basic-37 | AGCACTTGTCGAGGGCGCCACTCTTGCTGCCACTCTTGGC | 654 |
| GAV Basic-38 | GAATAAGCGCTTCCCGACTGGCCAAGAGTGGCAGCAAGAG | 655 |
| GAV Basic-39 | CAGTCGGGAAGCGCTTATTCATCTGTTGCTCCCCAGGTTT | 656 |
| GAV Basic-40 | GAATCGTTGGAGAAAGCACAAAACCTGGGGAGCAACAGAT | 657 |
| GAV Basic-41 | TGTGCTTTCTCCAACGATTCTGGGTGTCGTCTGGTGGATA | 658 |
| GAV Basic-42 | TGTTGATGTTGGAGTCGACGTATCCACCAGACGACACCCA | 659 |
| GAV Basic-43 | CGTCGACTCCAACATCAACACCAACGAGGGCAGGACTGGC | 660 |
| GAV Basic-44 | AGGACGGAGTTGACATCCTTGCCAGTCCTGCCCTCGTTGG | 661 |
| GAV Basic-45 | AAGGATGTCAACTCCGTCCTGACTTCCATCCACACCTTCG | 662 |
| GAV Basic-46 | GTCACAGCCAAGGTTGGGATCGAAGGTGTGGATGGAAGTC | 663 |
| GAV Basic-47 | ATCCCAACCTTGGCTGTGACGCAGGCACCTTCCAGCCATG | 664 |
| GAV Basic-48 | TGGAGAGCGCTTTGTCACTGCATGGCTGGAAGGTGCCTGC | 665 |
| GAV Basic-49 | CAGTGACAAAGCGCTCTCCAACCTCAAGGTTGTTGTCGAC | 666 |
| GAV Basic-50 | CCGTAGATGGAGCGGAAGGAGTCGACAACAACCTTGAGGT | 667 |
| GAV Basic-51 | TCCTTCCGCTCCATCTACGGCGTGAACAAGGGCATTCCTG | 668 |
| GAV Basic-52 | AATGGCGACGGCAGCACCGGCAGGAATGCCCTTGTTCACG | 669 |
| GAV Basic-53 | CCGGTGCTGCCGTCGCCATTGGCCGGTATGCAGAGGATGT | 670 |
| GAV Basic-54 | AAGGGTTGCCGTTGTAGTACACATCCTCTGCATACCGGCC | 671 |
| GAV Basic-55 | GTACTACAACGGCAACCCTTGGTATCTTGCTACATTTGCT | 672 |
| GAV Basic-56 | TCGTACAGCTGCTCGGCAGCAGCAAATGTAGCAAGATACC | 673 |
| GAV Basic-57 | GCTGCCGAGCAGCTGTACGATGCCATCTACGTCTGGAAGA | 674 |
| GAV Basic-58 | CACCGTGATGGAGCCCGTCTTCTTCCAGACGTAGATGGCA | 675 |
| GAV Basic-59 | AGACGGGCTCCATCACGGTGACCGCCACCTCCCTGGCCTT | 676 |
| GAV Basic-60 | CAGGAACAAGCTCCTGGAAGAAGGCCAGGGAGGTGGCGGT | 677 |
| GAV Basic-61 | CTTCCAGGAGCTTGTTCCTGGCGTGACGGCCGGGACCTAC | 678 |
| GAV Basic-62 | AAGGTCGAAGAGCTGCTGGAGTAGGTCCCGGCCGTCACGC | 679 |
| GAV Basic-63 | TCCAGCAGCTCTTCGACCTTTACCAACATCATCAACGCCG | 680 |
| GAV Basic-64 | GCCATCGGCGTATGTCGAGACGGCGTTGATGATGTTGGTA | 681 |
| GAV Basic-65 | TCTCGACATACGCCGATGGCTTCCTCAGCGAGGCTGCCAA | 682 |
| GAV Basic-66 | AACCGTCGGCGGGGACGTACTTGGCAGCCTCGCTGAGGAA | 683 |
| GAV Basic-67 | GTACGTCCCCGCCGACGGTTCGCTGGCCGAGCAGTTTGAC | 684 |
| GAV Basic-68 | AGCGGAGTGCCGCTGTTGCGGTCAAACTGCTCGGCCAGCG | 685 |
| GAV Basic-69 | CGCAACAGCGGCACTCCGCTGTCTGCGCTTCACCTGACGT | 686 |
| GAV Basic-70 | CAAGAACGAGGCGTACGACCACGTCAGGTGAAGCGCAGAC | 687 |
| GAV Basic-71 | GGTCGTACGCCTCGTTCTTGACAGCCACGGCCCGTCGGGC | 688 |
| GAV Basic-72 | ACGAGGGGGGCACGATGCCAGCCCGACGGGCCGTGGCTGT | 689 |
| GAV Basic-73 | TGGCATCGTGCCCCCCTCGTGGGCCAACAGCAGCGCTAGC | 690 |
| GAV Basic-74 | GAGCACGTCGAGGGGATCGTGCTAGCGCTGCTGTTGGCCC | 691 |
| GAV Basic-75 | ACGATCCCCTCGACGTGCTCCGGCGCGTCCGTGGTCGGAT | 692 |
| GAV Basic-76 | GGCGGTGGGACGCGAGTAGGATCCGACCACGGACGCGCCG | 693 |
| GAV Basic-77 | CCTACTCGCGTCCCACCGCCACGTCATTCCCTCCGTCGCA | 694 |
| GAV Basic-78 | GCACGCCAGGCTTGGGCGTCTGCGACGGAGGGAATGACGT | 695 |
| GAV Basic-79 | GACGCCCAAGCCTGGCGTGCCTTCCGGTACTCCCTACACG | 696 |
| GAV Basic-80 | GGGGTCGCGCAGGGCAGGGGCGTGTAGGGAGTACCGGAAG | 697 |
| GAV Basic-81 | CCCCTGCCCTGCGCGACCCCAACCTCCGTGGCCGTCACCT | 698 |
| GAV Basic-82 | TGTCGACACGAGCTCGTGGAAGGTGACGGCCACGGAGGTT | 699 |
| GAV Basic-83 | TCCACGAGCTCGTGTCGACACAGTTTGGCCAGACGGTCAA | 700 |
| GAV Basic-84 | CCGCGGCGTTGCCCGCCACCTTGACCGTCTGGCCAAACTG | 701 |
| GAV Basic-85 | GGTGGCGGGCAACGCCGCGGCCCTGGGCAACTGGAGCACG | 702 |
| GAV Basic-86 | TCCAGAGCCACGGCGGCGCTCGTGCTCCAGTTGCCCAGGG | 703 |
| GAV Basic-87 | AGCGCCGCCGTGGCTCTGGACGCCGTCAACTATGCCGATA | 704 |
| GAV Basic-88 | CCCAATCCACAGGGGGTGGTTATCGGCATAGTTGACGGCG | 705 |
| GAV Basic-89 | ACCACCCCCTGTGGATTGGGACGGTCAACCTCGAGGCTGG | 706 |
| GAV Basic-90 | ACTTGTACTCCACGACGTCTCCAGCCTCGAGGTTGACCGT | 707 |
| GAV Basic-91 | AGACGTCGTGGAGTACAAGTACATCAATGTGGGCCAAGAT | 708 |
| GAV Basic-92 | CTCTCCCAGGTCACGGAGCCATCTTGGCCCACATTGATGT | 709 |
| GAV Basic-93 | GGCTCCGTGACCTGGGAGAGTGATCCCAACCACACTTACA | 710 |
| GAV Basic-94 | ACAAGCCACCGCAGGAACCGTGTAAGTGTGGTTGGGATCA | 711 |
| GAV Basic-95 | CGGTTCCTGCGGTGGCTTGTGTGACGCAGGTTGTCAAGGA | 712 |
| GAV Basic-96 | TTTACGACTGCCAGGTGTCCTCCTTGACAACCTGCGTCAC | 713 |
| GAV Basic-97 | GGACACCTGGCAGTCGTAAACCCAGCTTTCTTGTACAAAG | 714 |
| GAV Basic-98 | ACCACTTTGTACAAGAAAGCTGGG | 715 |
| A111-13 | TGCGGTGATTGCATCTCCCAGCACACTTTGCCCGGACTAC | 716 |
| A111-14 | TCTCGCGTCCACATGTAATAGTAGTCCGGGCAAAGTGTGC | 717 |
| A111-69 | CGCAACAGCGGCACTCCGCTGTCTGCGGTTCACCTGACGT | 718 |
| A111-70 | CAAGAACGAGGCGTACGACCACGTCAGGTGAACCGCAGAC | 719 |
| A111-82 | TGTCGACACGAGCGCGTGGAAGGTGACGGCCACGGAGGTT | 720 |
| A111-83 | TCCACGCGCTCGTGTCGACACAGTTTGGCCACACGGTCAA | 721 |
| A111-84 | CCGCGGCGTTGCCCGCCACCTTGACCGTGTGGCCAAACTG | 722 |
| A111-87 | AGCGCCGCCGTGGCTCTGGACGCCGTCAACTATAGAGATA | 723 |
| A111-88 | CCCAATCCACAGGGGGTGGTTATCTCTATAGTTGACGGCG | 724 |
| A112-13 | TGCGGTGATTGCATCTCCCAGCACACTTGACCCGGACTAC | 725 |
| A112-14 | TCTCGCGTCCACATGTAATAGTAGTCCGGGTCAAGTGTGC | 726 |
| A112-69 | CGCAACAGCGGCACTCCGCTGTCTGCGGTTCACCTGACGT | 727 |
| A112-70 | CAAGAACGAGGCGTACGACCACGTCAGGTGAACCGCAGAC | 728 |
| A112-82 | TGTCGACACGAGCGCGTGGAAGGTGACGGCCACGGAGGTT | 729 |
| A112-83 | TCCACGCGCTCGTGTCGACACAGTTTGGCCACACGGTCAA | 730 |
| A112-84 | CCGCGGCGTTGCCCGCCACCTTGACCGTGTGGCCAAACTG | 731 |
| A112-87 | AGCGCCGCCGTGGCTCTGGACGCCGTCAACTATAGAGATA | 732 |
| A112-88 | CCCAATCCACAGGGGGTGGTTATCTCTATAGTTGACGGCG | 733 |
| A113-13 | TGCGGTGATTGCATCTCCCAGCACACTTTGCCCGGACTAC | 734 |
| A113-14 | TCTCGCGTCCACATGTAATAGTAGTCCGGGCAAAGTGTGC | 735 |
| A113-16 | GGTGAAGCGGTCGATGAGGATCTTGAAGACAAGAGCGCTA | 736 |
| A113-17 | TCCTCATCGACCGCTTCACCGAAACGTACGATGCGGGCCT | 737 |
| A113-69 | CGCAACAGCGGCACTCCGCTGTCTGCGGTTCACCTGACGT | 738 |
| A113-70 | CAAGAACGAGGCGTACGACCACGTCAGGTGAACCGCAGAC | 739 |
| A113-82 | TGTCGACACGAGCGCGTGGAAGGTGACGGCCACGGAGGTT | 740 |
| A113-83 | TCCACGCGCTCGTGTCGACACAGTTTGGCCACACGGTCAA | 741 |
| A113-84 | CCGCGGCGTTGCCCGCCACCTTGACCGTGTGGCCAAACTG | 742 |
| A113-87 | AGCGCCGCCGTGGCTCTGGACGCCGTCAACTATAGAGATA | 743 |
| A113-88 | CCCAATCCACAGGGGGTGGTTATCTCTATAGTTGACGGCG | 744 |
| A114-13 | TGCGGTGATTGCATCTCCCAGCACACTTGACCCGGACTAC | 745 |
| A114-14 | TCTCGCGTCCACATGTAATAGTAGTCCGGGTCAAGTGTGC | 746 |
| A114-16 | GGTGAAGCGGTCGATGAGGATCTTGAAGACAAGAGCGCTA | 747 |
| A114-17 | TCCTCATCGACCGCTTCACCGAAACGTACGATGCGGGCCT | 748 |
| A114-69 | CGCAACAGCGGCACTCCGCTGTCTGCGGTTCACCTGACGT | 749 |
| A114-70 | CAAGAACGAGGCGTACGACCACGTCAGGTGAACCGCAGAC | 750 |
| A114-82 | TGTCGACACGAGCGCGTGGAAGGTGACGGCCACGGAGGTT | 751 |
| A114-83 | TCCACGCGCTCGTGTCGACACAGTTTGGCCACACGGTCAA | 752 |
| A114-84 | CCGCGGCGTTGCCCGCCACCTTGACCGTGTGGCCAAACTG | 753 |
| A114-87 | AGCGCCGCCGTGGCTCTGGACGCCGTCAACTATAGAGATA | 754 |
| A114-88 | CCCAATCCACAGGGGGTGGTTATCTCTATAGTTGACGGCG | 755 |
| A115-13 | TGCGGTGATTGCATCTCCCAGCACAAGAGACCCGGACTAC | 756 |
| A115-14 | TCTCGCGTCCACATGTAATAGTAGTCCGGGTCTCTTGTGC | 757 |
| A115-69 | CGCAACAGCGGCACTCCGCTGTCTGCGGTTCACCTGACGT | 758 |
| A115-70 | CAAGAACGAGGCGTACGACCACGTCAGGTGAACCGCAGAC | 759 |
| A115-82 | TGTCGACACGAGCGCGTGGAAGGTGACGGCCACGGAGGTT | 760 |
| A115-83 | TCCACGCGCTCGTGTCGACACAGTTTGGCCACACGGTCAA | 761 |
| A115-84 | CCGCGGCGTTGCCCGCCACCTTGACCGTGTGGCCAAACTG | 762 |
| A115-87 | AGCGCCGCCGTGGCTCTGGACGCCGTCAACTATAGAGATA | 763 |
| A115-88 | CCCAATCCACAGGGGGTGGTTATCTCTATAGTTGACGGCG | 764 |
| A116-13 | TGCGGTGATTGCATCTCCCAGCACAAGAGACCCGGACTAC | 765 |
| A116-14 | TCTCGCGTCCACATGTAATAGTAGTCCGGGTCTCTTGTGC | 766 |
| A116-16 | GGTGAAGCGGTCGATGAGGATCTTGAAGACAAGAGCGCTA | 767 |
| A116-17 | TCCTCATCGACCGCTTCACCGAAACGTACGATGCGGGCCT | 768 |
| A116-69 | CGCAACAGCGGCACTCCGCTGTCTGCGGTTCACCTGACGT | 769 |
| A116-70 | CAAGAACGAGGCGTACGACCACGTCAGGTGAACCGCAGAC | 770 |
| A116-82 | TGTCGACACGAGCGCGTGGAAGGTGACGGCCACGGAGGTT | 771 |
| A116-83 | TCCACGCGCTCGTGTCGACACAGTTTGGCCACACGGTCAA | 772 |
| A116-84 | CCGCGGCGTTGCCCGCCACCTTGACCGTGTGGCCAAACTG | 773 |
| A116-87 | AGCGCCGCCGTGGCTCTGGACGCCGTCAACTATAGAGATA | 774 |
| A116-88 | CCCAATCCACAGGGGGTGGTTATCTCTATAGTTGACGGCG | 775 |
| A117-13 | TGCGGTGATTGCATCTCCCAGCACAAGAGACCCGGACTAC | 776 |
| A117-14 | TCTCGCGTCCACATGTAATAGTAGTCCGGGTCTCTTGTGC | 777 |
| A117-69 | CGCAACAGCGGCACTCCGCTGTCTGCGAGACACCTGACGT | 778 |
| A117-70 | CAAGAACGAGGCGTACGACCACGTCAGGTGTCTCGCAGAC | 779 |
| A117-82 | TGTCGACACGAGCGCGTGGAAGGTGACGGCCACGGAGGTT | 780 |
| A117-83 | TCCACGCGCTCGTGTCGACACAGTTTGGCCACACGGTCAA | 781 |
| A117-84 | CCGCGGCGTTGCCCGCCACCTTGACCGTGTGGCCAAACTG | 782 |
| A117-87 | AGCGCCGCCGTGGCTCTGGACGCCGTCAACTATAGAGATA | 783 |
| A117-88 | CCCAATCCACAGGGGGTGGTTATCTCTATAGTTGACGGCG | 784 |
| A118-13 | TGCGGTGATTGCATCTCCCAGCACAAGAGACCCGGACTAC | 785 |
| A118-14 | TCTCGCGTCCACATGTAATAGTAGTCCGGGTCTCTTGTGC | 786 |
| A118-16 | GGTGAAGCGGTCGATGAGGATCTTGAAGACAAGAGCGCTA | 787 |
| A118-17 | TCCTCATCGACCGCTTCACCGAAACGTACGATGCGGGCCT | 788 |
| A118-69 | CGCAACAGCGGCACTCCGCTGTCTGCGAGACACCTGACGT | 789 |
| A118-70 | CAAGAACGAGGCGTACGACCACGTCAGGTGTCTCGCAGAC | 790 |
| A118-82 | TGTCGACACGAGCGCGTGGAAGGTGACGGCCACGGAGGTT | 791 |
| A118-83 | TCCACGCGCTCGTGTCGACACAGTTTGGCCACACGGTCAA | 792 |
| A118-84 | CCGCGGCGTTGCCCGCCACCTTGACCGTGTGGCCAAACTG | 793 |
| A118-87 | AGCGCCGCCGTGGCTCTGGACGCCGTCAACTATAGAGATA | 794 |
| A118-88 | CCCAATCCACAGGGGGTGGTTATCTCTATAGTTGACGGCG | 795 |
| CS1-69 | CGCAACAGCGGCACTCCGCTGTCTGCGGTTCACCTGACGT | 796 |
| CS1-70 | CAAGAACGAGGCGTACGACCACGTCAGGTGAACCGCAGAC | 797 |
| CS1-71 | GGTCGTACGCCTCGTTCTTGACAGCCGCGCTCCGTCGGGC | 798 |
| CS1-72 | ACGAGGGGGGCACGATGCCAGCCCGACGGAGCGCGGCTGT | 799 |
| CS1-83 | TCCACGAGCTCGTGTCGACACAGTTTGGCCACACGGTCAA | 800 |
| CS1-84 | CCGCGGCGTTGCCCGCCACCTTGACCGTGTGGCCAAACTG | 801 |
| CS1-87 | AGCGCCGCCGTGGCTCTGGACCGCGTCAACTATCGCGATA | 802 |
| CS1-88 | CCCAATCCACAGGGGGTGGTTATCGCGATAGTTGACGCGG | 803 |
| CS1-91 | AGACGTCGTGGAGTACAAGTACATCATTGTGGGCCAAGAT | 804 |
| CS1-92 | CTCTCCCAGGTCACGGAGCCATCTTGGCCCACAATGATGT | 805 |
| CS2-69 | CGCAACAGCGGCACTCCGCTGTCTGCGGTTCACCTGACGT | 806 |
| CS2-70 | CAAGAACGAGGCGTACGACCACGTCAGGTGAACCGCAGAC | 807 |
| CS2-71 | GGTCGTACGCCTCGTTCTTGACAGCCGCGCAACGTCGGGC | 808 |
| CS2-72 | ACGAGGGGGGCACGATGCCAGCCCGACGTTGCGCGGCTGT | 809 |
| CS2-83 | TCCACGAGCTCGTGTCGACACAGTTTGGCCACACGGTCAA | 810 |
| CS2-84 | CCGCGGCGTTGCCCGCCACCTTGACCGTGTGGCCAAACTG | 811 |
| CS2-87 | AGCGCCGCCGTGGCTCTGGACCGCGTCAACTATCGCGATA | 812 |
| CS2-88 | CCCAATCCACAGGGGGTGGTTATCGCGATAGTTGACGCGG | 813 |
| CS2-91 | AGACGTCGTGGAGTACAAGTACATCATTGTGGGCCAAGAT | 814 |
| CS2-92 | CTCTCCCAGGTCACGGAGCCATCTTGGCCCACAATGATGT | 815 |
| CS3-69 | CGCAACAGCGGCACTCCGCTGTCTGCGGTTCACCTGACGT | 816 |
| CS3-70 | CAAGAACGAGGCGTACGACCACGTCAGGTGAACCGCAGAC | 817 |
| CS3-71 | GGTCGTACGCCTCGTTCTTGACAGCCGCGGCCCGTCGGGC | 818 |
| CS3-72 | ACGAGGGGGGCACGATGCCAGCCCGACGGGCCGCGGCTGT | 819 |
| CS3-83 | TCCACGAGCTCGTGTCGACACAGTTTGGCCACACGGTCAA | 820 |
| CS3-84 | CCGCGGCGTTGCCCGCCACCTTGACCGTGTGGCCAAACTG | 821 |
| CS3-87 | AGCGCCGCCGTGGCTCTGGACCGCGTCAACTATGCCGATA | 822 |
| CS3-88 | CCCAATCCACAGGGGGTGGTTATCGGCATAGTTGACGCGG | 823 |
| CS3-91 | AGACGTCGTGGAGTACAAGTACATCATTGTGGGCCAAGAT | 824 |
| CS3-92 | CTCTCCCAGGTCACGGAGCCATCTTGGCCCACAATGATGT | 825 |
| CS4-69 | CGCAACAGCGGCACTCCGCTGTCTGCGGTTCACCTGACGT | 826 |
| CS4-70 | CAAGAACGAGGCGTACGACCACGTCAGGTGAACCGCAGAC | 827 |
| CS4-71 | GGTCGTACGCCTCGTTCTTGACAGCCGCGGCCCGTCGGGC | 828 |
| CS4-72 | ACGAGGGGGGCACGATGCCAGCCCGACGGGCCGCGGCTGT | 829 |
| CS4-83 | TCCACGAGCTCGTGTCGACACAGTTTGGCCACACGGTCAA | 830 |
| CS4-84 | CCGCGGCGTTGCCCGCCACCTTGACCGTGTGGCCAAACTG | 831 |
| CS4-87 | AGCGCCGCCGTGGCTCTGGACGCCGTCAACTATCGCGATA | 832 |
| CS4-88 | CCCAATCCACAGGGGGTGGTTATCGCGATAGTTGACGGCG | 833 |
| CS4-91 | AGACGTCGTGGAGTACAAGTACATCATTGTGGGCCAAGAT | 834 |
| CS4-92 | CTCTCCCAGGTCACGGAGCCATCTTGGCCCACAATGATGT | 835 |
| LQ1-50 | CAGTAGATGGAGCGGAAGGAGTCGACAACAACCTTGAGGT | 836 |
| LQ1-51 | TCCTTCCGCTCCATCTACTGCGTGAACAAGGGCATTCCTG | 837 |
| LQ1-69 | CGCAACAGCGGCACTCCGCTGTCTGCGAGACACCTGACGT | 838 |
| LQ1-70 | CAAGAACGAGGCGTACGACCACGTCAGGTGTCTCGCAGAC | 839 |
| LQ1-71 | GGTCGTACGCCTCGTTCTTGACAGCCACGCTCCGTCGGGC | 840 |
| LQ1-72 | ACGAGGGGGGCACGATGCCAGCCCGACGGAGCGTGGCTGT | 841 |
| LQ2-50 | CAGTAGATGGAGCGGAAGGAGTCGACAACAACCTTGAGGT | 842 |
| LQ2-51 | TCCTTCCGCTCCATCTACTGCGTGAACAAGGGCATTCCTG | 843 |
| LQ2-69 | CGCAACAGCGGCACTCCGCTGTCTGCGGTACACCTGACGT | 844 |
| LQ2-70 | CAAGAACGAGGCGTACGACCACGTCAGGTGTACCGCAGAC | 845 |
| LQ2-71 | GGTCGTACGCCTCGTTCTTGACAGCCACGCAGCGTCGGGC | 846 |
| LQ2-72 | ACGAGGGGGGCACGATGCCAGCCCGACGCTGCGTGGCTGT | 847 |
| LQ3-50 | CAGTAGATGGAGCGGAAGGAGTCGACAACAACCTTGAGGT | 848 |
| LQ3-51 | TCCTTCCGCTCCATCTACTGCGTGAACAAGGGCATTCCTG | 849 |
| LQ3-69 | CGCAACAGCGGCACTCCGCTGTCTGCGGTACACCTGACGT | 850 |
| LQ3-70 | CAAGAACGAGGCGTACGACCACGTCAGGTGTACCGCAGAC | 851 |
| LQ3-71 | GGTCGTACGCCTCGTTCTTGACAGCCACGTTACGTCGGGC | 852 |
| LQ3-72 | ACGAGGGGGGCACGATGCCAGCCCGACGTAACGTGGCTGT | 853 |
| LQ3-83 | TCCACGAGCTCGTGTCGACACAGTTTGGCCACACGGTCAA | 854 |
| LQ3-84 | CCGCGGCGTTGCCCGCCACCTTGACCGTGTGGCCAAACTG | 855 |
| LQ4-50 | CAGTAGATGGAGCGGAAGGAGTCGACAACAACCTTGAGGT | 856 |
| LQ4-51 | TCCTTCCGCTCCATCTACTGCGTGAACAAGGGCATTCCTG | 857 |
| LQ4-69 | CGCAACAGCGGCACTCCGCTGTCTGCGAGACACCTGACGT | 858 |
| LQ4-70 | CAAGAACGAGGCGTACGACCACGTCAGGTGTCTCGCAGAC | 859 |
| LQ4-71 | GGTCGTACGCCTCGTTCTTGACAGCCACGCAGCGTCGGGC | 860 |
| LQ4-72 | ACGAGGGGGGCACGATGCCAGCCCGACGCTGCGTGGCTGT | 861 |
| LQ4-83 | TCCACGAGCTCGTGTCGACACAGTTTGGCCACACGGTCAA | 862 |
| LQ4-84 | CCGCGGCGTTGCCCGCCACCTTGACCGTGTGGCCAAACTG | 863 |
| LQ5-69 | CGCAACAGCGGCACTCCGCTGTCTGCGCGTCACCTGACGT | 864 |
| LQ5-70 | CAAGAACGAGGCGTACGACCACGTCAGGTGACGCGCAGAC | 865 |
| LQ5-71 | GGTCGTACGCCTCGTTCTTGACAGCCACGCTCCGTCGGGC | 866 |
| LQ5-72 | ACGAGGGGGGCACGATGCCAGCCCGACGGAGCGTGGCTGT | 867 |
| LQ5-83 | TCCACGAGCTCGTGTCGACACAGTTTGGCCACACGGTCAA | 868 |
| LQ5-84 | CCGCGGCGTTGCCCGCCACCTTGACCGTGTGGCCAAACTG | 869 |
| LQ6-69 | CGCAACAGCGGCACTCCGCTGTCTGCGGTTCACCTGACGT | 870 |
| LQ6-70 | CAAGAACGAGGCGTACGACCACGTCAGGTGAACCGCAGAC | 871 |
| LQ6-71 | GGTCGTACGCCTCGTTCTTGACAGCCACGCTTCGTCGGGC | 872 |
| LQ6-72 | ACGAGGGGGGCACGATGCCAGCCCGACGAAGCGTGGCTGT | 873 |
| LQ6-83 | TCCACGAGCTCGTGTCGACACAGTTTGGCCATACGGTCAA | 874 |
| LQ6-84 | CCGCGGCGTTGCCCGCCACCTTGACCGTATGGCCAAACTG | 875 |
| TS1-13 | TGCGGTGATTGCATCTCCCAGCACAAGAGACCCGGACTAC | 876 |
| TS1-14 | TCTCGCGTCCACATGTAATAGTAGTCCGGGTCTCTTGTGC | 877 |
| TS1-71 | GGTCGTACGCCTCGTTCTTGACAGCCGCAGCCCGTCGGGC | 878 |
| TS1-72 | ACGAGGGGGGCACGATGCCAGCCCGACGGGCTGCGGCTGT | 879 |
| TS1-82 | TGTCGACACGAGCACGTGGAAGGTGACGGCCACGGAGGTT | 880 |
| TS1-83 | TCCACGTGCTCGTGTCGACACAGTTTGGCCAGACGGTCAA | 881 |
| TS1-87 | AGCGCCGCCGTGGCTCTGGACCGCGTCAACTATGCCGATA | 882 |
| TS1-88 | CCCAATCCACAGGGGGTGGTTATCGGCATAGTTGACGCGG | 883 |
| TS1-91 | AGACGTCGTGGAGTACAAGTACATCAAAGTGGGCCAAGAT | 884 |
| TS1-92 | CTCTCCCAGGTCACGGAGCCATCTTGGCCCACTTTGATGT | 885 |
| TS2-13 | TGCGGTGATTGCATCTCCCAGCACAATTAGACCGGACTAC | 886 |
| TS2-14 | TCTCGCGTCCACATGTAATAGTAGTCCGGTCTAATTGTGC | 887 |
| TS2-82 | TGTCGACACGAGCGCGTGGAAGGTGACGGCCACGGAGGTT | 888 |
| TS2-83 | TCCACGCGCTCGTGTCGACACAGTTTGGCCACACGGTCAA | 889 |
| TS2-84 | CCGCGGCGTTGCCCGCCACCTTGACCGTGTGGCCAAACTG | 890 |
| TS2-91 | AGACGTCGTGGAGTACAAGTACATCATTGTGGGCCAAGAT | 891 |
| TS2-92 | CTCTCCCAGGTCACGGAGCCATCTTGGCCCACAATGATGT | 892 |
| TS3-82 | TGTCGACACGAGCGCGTGGAAGGTGACGGCCACGGAGGTT | 893 |
| TS3-83 | TCCACGCGCTCGTGTCGACACAGTTTGGCCAGACGGTCAA | 894 |
| TS3-91 | AGACGTCGTGGAGTACAAGTACATCATTGTGGGCCAAGAT | 895 |
| TS3-92 | CTCTCCCAGGTCACGGAGCCATCTTGGCCCACAATGATGT | 896 |
| TS4-13 | TGCGGTGATTGCATCTCCCAGCACAAGAGACCCGGACTAC | 897 |
| TS4-14 | TCTCGCGTCCACATGTAATAGTAGTCCGGGTCTCTTGTGC | 898 |
| TS4-71 | GGTCGTACGCCTCGTTCTTGACAGCCGCGGCCCGTCGGGC | 899 |
| TS4-72 | ACGAGGGGGGCACGATGCCAGCCCGACGGGCCGCGGCTGT | 900 |
| TS4-82 | TGTCGACACGAGCGCGTGGAAGGTGACGGCCACGGAGGTT | 901 |
| TS4-83 | TCCACGCGCTCGTGTCGACACAGTTTGGCCACACGGTCAA | 902 |
| TS4-84 | CCGCGGCGTTGCCCGCCACCTTGACCGTGTGGCCAAACTG | 903 |
| TS4-91 | AGACGTCGTGGAGTACAAGTACATCATTGTGGGCCAAGAT | 904 |
| TS4-92 | CTCTCCCAGGTCACGGAGCCATCTTGGCCCACAATGATGT | 905 |
| TS5-13 | TGCGGTGATTGCATCTCCCAGCACAATTCGCCCGGACTAC | 906 |
| TS5-14 | TCTCGCGTCCACATGTAATAGTAGTCCGGGCGAATTGTGC | 907 |
| TS5-71 | GGTCGTACGCCTCGTTCTTGACAGCCGCGGCCCGTCGGGC | 908 |
| TS5-72 | ACGAGGGGGGCACGATGCCAGCCCGACGGGCCGCGGCTGT | 909 |
| TS5-83 | TCCACGAGCTCGTGTCGACACAGTTTGGCCACACGGTCAA | 910 |
| TS5-84 | CCGCGGCGTTGCCCGCCACCTTGACCGTGTGGCCAAACTG | 911 |
| TS5-87 | AGCGCCGCCGTGGCTCTGGACGCGGTCAACTATGCCGATA | 912 |
| TS5-88 | CCCAATCCACAGGGGGTGGTTATCGGCATAGTTGACCGCG | 913 |
| GAV st-1 | GAGAGGGGACAAGTTTGTACAAAAAAGCAGGCT | 914 |
| GAV st-2 | GCAGTCGACAGGACGTGCATAGCCTGCTTTTTTGTACAAA | 915 |
| GAV st-3 | ATGCACGTCCTGTCGACTGCGGTGCTGCTCGGCTCCGTTG | 916 |
| GAV st-4 | TCCCAGGACCTTTTGAACGGCAACGGAGCCGAGCAGCACC | 917 |
| GAV st-5 | CCGTTCAAAAGGTCCTGGGAAGACCAGGATCAAGCGGTCT | 918 |
| GAV st-6 | ACCTCTTGGTGACGTCGGACAGACCGCTTGATCCTGGTCT | 919 |
| GAV st-7 | GTCCGACGTCACCAAGAGGTCTGTTGACGACTTCATCAGC | 920 |
| GAV st-8 | AGTGCAATAGGCGTCTCGGTGCTGATGAAGTCGTCAACAG | 921 |
| GAV st-9 | ACCGAGACGCCTATTGCACTGAACAATCTTCTTTGCAATG | 922 |
| GAV st-10 | ACGGCATCCATCAGGACCAACATTGCAAAGAAGATTGTTC | 923 |
| GAV st-11 | TTGGTCCTGATGGATGCCGTGCATTCGGCACATCAGCTGG | 924 |
| GAV st-12 | TGGGAGATGCAATCACCGCACCAGCTGATGTGCCGAATGC | 925 |
| GAV st-13 | TGCGGTGATTGCATCTCCCAGCACACAAGACCCGGACTAC | 926 |
| GAV st-14 | TCTCGCGTCCACATGTAATAGTAGTCCGGGTCTTGTGTGC | 927 |
| GAV st-15 | TATTACATGTGGACGCGAGATAGCGCTCTTGTCTTCAAGA | 928 |
| GAV st-16 | GGTGAAGCGGTCGATGAGGTTCTTGAAGACAAGAGCGCTA | 929 |
| GAV st-17 | ACCTCATCGACCGCTTCACCGAAACGTACGATGCGGGCCT | 930 |
| GAV st-18 | ACTGCTCGATGCGGCGCTGCAGGCCCGCATCGTACGTTTC | 931 |
| GAV st-19 | GCAGCGCCGCATCGAGCAGTACATTACTGCCCAGGTCACT | 932 |
| GAV st-20 | GGGTTAGAGAGGCCCTGGAGAGTGACCTGGGCAGTAATGT | 933 |
| GAV st-21 | CTCCAGGGCCTCTCTAACCCCTCGGGCTCCCTCGCGGACG | 934 |
| GAV st-22 | GGGCTCGCCGAGACCAGAGCCGTCCGCGAGGGAGCCCGAG | 935 |
| GAV st-23 | GCTCTGGTCTCGGCGAGCCCAAGTTTGAGTTGACCCTGAA | 936 |
| GAV st-24 | CCCAGTTGCCGGTGAAAGGCTTCAGGGTCAACTCAAACTT | 937 |
| GAV st-25 | GCCTTTCACCGGCAACTGGGGTCGACCGCAGCGGGATGGC | 938 |
| GAV st-26 | GCAATGGCTCGCAGAGCTGGGCCATCCCGCTGCGGTCGAC | 939 |
| GAV st-27 | CCAGCTCTGCGAGCCATTGCCTTGATTGGATACTCAAAGT | 940 |
| GAV st-28 | ATAGTTGTTGTTGATGAGCCACTTTGAGTATCCAATCAAG | 941 |
| GAV st-29 | GGCTCATCAACAACAACTATCAGTCGACTGTGTCCAACGT | 942 |
| GAV st-30 | TGCGCACAATAGGCCAGATGACGTTGGACACAGTCGACTG | 943 |
| GAV st-31 | CATCTGGCCTATTGTGCGCAACGACCTCAACTATGTTGCC | 944 |
| GAV st-32 | CCGGTTTGGTTCCAGTACTGGGCAACATAGTTGAGGTCGT | 945 |
| GAV st-33 | CAGTACTGGAACCAAACCGGCTTTGACCTCTGGGAAGAAG | 946 |
| GAV st-34 | AAAGAATGAGCTCCCATTGACTTCTTCCCAGAGGTCAAAG | 947 |
| GAV st-35 | TCAATGGGAGCTCATTCTTTACTGTTGCCAACCAGCACCG | 948 |
| GAV st-36 | TGGCGCCCTCGACAAGTGCTCGGTGCTGGTTGGCAACAGT | 949 |
| GAV st-37 | AGCACTTGTCGAGGGCGCCACTCTTGCTGCCACTCTTGGC | 950 |
| GAV st-38 | GAATAAGCGCTTCCCGACTGGCCAAGAGTGGCAGCAAGAG | 951 |
| GAV st-39 | CAGTCGGGAAGCGCTTATTCATCTGTTGCTCCCCAGGTTT | 952 |
| GAV st-40 | GAATCGTTGGAGAAAGCACAAAACCTGGGGAGCAACAGAT | 953 |
| GAV st-41 | TGTGCTTTCTCCAACGATTCTGGGTGTCGTCTGGTGGATA | 954 |
| GAV st-42 | TGTTGATGTTGGAGTCGACGTATCCACCAGACGACACCCA | 955 |
| GAV st-43 | CGTCGACTCCAACATCAACACCAACGAGGGCAGGACTGGC | 956 |
| GAV st-44 | AGGACGGAGTTGACATCCTTGCCAGTCCTGCCCTCGTTGG | 957 |
| GAV st-45 | AAGGATGTCAACTCCGTCCTGACTTCCATCCACACCTTCG | 958 |
| GAV st-46 | GTCACAGCCAAGGTTGGGATCGAAGGTGTGGATGGAAGTC | 959 |
| GAV st-47 | ATCCCAACCTTGGCTGTGACGCAGGCACCTTCCAGCCATG | 960 |
| GAV st-48 | TGGAGAGCGCTTTGTCACTGCATGGCTGGAAGGTGCCTGC | 961 |
| GAV st-49 | CAGTGACAAAGCGCTCTCCAACCTCAAGGTTGTTGTCGAC | 962 |
| GAV st-50 | CCGTAGATGGAGCGGAAGGAGTCGACAACAACCTTGAGGT | 963 |
| GAV st-51 | TCCTTCCGCTCCATCTACGGCGTGAACAAGGGCATTCCTG | 964 |
| GAV st-52 | AATGGCGACGGCAGCACCGGCAGGAATGCCCTTGTTCACG | 965 |
| GAV st-53 | CCGGTGCTGCCGTCGCCATTGGCCGGTATGCAGAGGATGT | 966 |
| GAV st-54 | AAGGGTTGCCGTTGTAGTACACATCCTCTGCATACCGGCC | 967 |
| GAV st-55 | GTACTACAACGGCAACCCTTGGTATCTTGCTACATTTGCT | 968 |
| GAV st-56 | TCGTACAGCTGCTCGGCAGCAGCAAATGTAGCAAGATACC | 969 |
| GAV st-57 | GCTGCCGAGCAGCTGTACGATGCCATCTACGTCTGGAAGA | 970 |
| GAV st-58 | CACCGTGATGGAGCCCGTCTTCTTCCAGACGTAGATGGCA | 971 |
| GAV st-59 | AGACGGGCTCCATCACGGTGACCGCCACCTCCCTGGCCTT | 972 |
| GAV st-60 | CAGGAACAAGCTCCTGGAAGAAGGCCAGGGAGGTGGCGGT | 973 |
| GAV st-61 | CTTCCAGGAGCTTGTTCCTGGCGTGACGGCCGGGACCTAC | 974 |
| GAV st-62 | AAGGTCGAAGAGCTGCTGGAGTAGGTCCCGGCCGTCACGC | 975 |
| GAV st-63 | TCCAGCAGCTCTTCGACCTTTACCAACATCATCAACGCCG | 976 |
| GAV st-64 | GCCATCGGCGTATGTCGAGACGGCGTTGATGATGTTGGTA | 977 |
| GAV st-65 | TCTCGACATACGCCGATGGCTTCCTCAGCGAGGCTGCCAA | 978 |
| GAV st-66 | AACCGTCGGCGGGGACGTACTTGGCAGCCTCGCTGAGGAA | 979 |
| GAV st-67 | GTACGTCCCCGCCGACGGTTCGCTGGCCGAGCAGTTTGAC | 980 |
| GAV st-68 | AGCGGAGTGCCGCTGTTGCGGTCAAACTGCTCGGCCAGCG | 981 |
| GAV st-69 | CGCAACAGCGGCACTCCGCTGTCTGCGCTTCACCTGACGT | 982 |
| GAV st-70 | CAAGAACGAGGCGTACGACCACGTCAGGTGAAGCGCAGAC | 983 |
| GAV st-71 | GGTCGTACGCCTCGTTCTTGACAGCCACGGCCCGTCGGGC | 984 |
| GAV st-72 | ACGAGGGGGGCACGATGCCAGCCCGACGGGCCGTGGCTGT | 985 |
| GAV st-73 | TGGCATCGTGCCCCCCTCGTGGGCCAACAGCAGCGCTAGC | 986 |
| GAV st-74 | GAGCACGTCGAGGGGATCGTGCTAGCGCTGCTGTTGGCCC | 987 |
| GAV st-75 | ACGATCCCCTCGACGTGCTCCGGCGCGTCCGTGGTCGGAT | 988 |
| GAV st-76 | GGCGGTGGGACGCGAGTAGGATCCGACCACGGACGCGCCG | 989 |
| GAV st-77 | CCTACTCGCGTCCCACCGCCACGTCATTCCCTCCGTCGCA | 990 |
| GAV st-78 | GCACGCCAGGCTTGGGCGTCTGCGACGGAGGGAATGACGT | 991 |
| GAV st-79 | GACGCCCAAGCCTGGCGTGCCTTCCGGTACTCCCTACACG | 992 |
| GAV st-80 | GGGGTCGCGCAGGGCAGGGGCGTGTAGGGAGTACCGGAAG | 993 |
| GAV st-81 | CCCCTGCCCTGCGCGACCCCAACCTCCGTGGCCGTCACCT | 994 |
| GAV st-82 | TGTCGACACGAGCTCGTGGAAGGTGACGGCCACGGAGGTT | 995 |
| GAV st-83 | TCCACGAGCTCGTGTCGACACAGTTTGGCCACACGGTCAA | 996 |
| GAV st-84 | CCGCGGCGTTGCCCGCCACCTTGACCGTGTGGCCAAACTG | 997 |
| GAV st-85 | GGTGGCGGGCAACGCCGCGGCCCTGGGCAACTGGAGCACG | 998 |
| GAV st-86 | TCCAGAGCCACGGCGGCGCTCGTGCTCCAGTTGCCCAGGG | 999 |
| GAV st-87 | AGCGCCGCCGTGGCTCTGGACGCCGTCAACTATGCCGATA | 1000 |
| GAV st-88 | CCCAATCCACAGGGGGTGGTTATCGGCATAGTTGACGGCG | 1001 |
| GAV st-89 | ACCACCCCCTGTGGATTGGGACGGTCAACCTCGAGGCTGG | 1002 |
| GAV st-90 | ACTTGTACTCCACGACGTCTCCAGCCTCGAGGTTGACCGT | 1003 |
| GAV st-91 | AGACGTCGTGGAGTACAAGTACATCAATGTGGGCCAAGAT | 1004 |
| GAV st-92 | CTCTCCCAGGTCACGGAGCCATCTTGGCCCACATTGATGT | 1005 |
| GAV st-93 | GGCTCCGTGACCTGGGAGAGTGATCCCAACCACACTTACA | 1006 |
| GAV st-94 | ACAAGCCACCGCAGGAACCGTGTAAGTGTGGTTGGGATCA | 1007 |
| GAV st-95 | CGGTTCCTGCGGTGGCTTGTGTGACGCAGGTTGTCAAGGA | 1008 |
| GAV st-96 | TTTACGACTGCCAGGTGTCCTCCTTGACAACCTGCGTCAC | 1009 |
| GAV st-97 | GGACACCTGGCAGTCGTAAACCCAGCTTTCTTGTACAAAG | 1010 |
| GAV st-98 | CTCTGGGGACCACTTTGTACAAGAAAGCTGGG | 1011 |
| RB1-16 | GGTGAAGCGGTCGATGAGGATCTTGAAGACAAGAGCGCTA | 1012 |
| RB1-17 | TCCTCATCGACCGCTTCACCGAAACGTACGATGCGGGCCT | 1013 |
| RB1-71 | GGTCGTACGCCTCGTTCTTGACAGCCGCGGCCCGTCGGGC | 1014 |
| RB1-72 | ACGAGGGGGGCACGATGCCAGCCCGACGGGCCGCGGCTGT | 1015 |
| RB2-69 | CGCAACAGCGGCACTCCGCTGTCTGCGGTTCACCTGACGT | 1016 |
| RB2-70 | CAAGAACGAGGCGTACGACCACGTCAGGTGAACCGCAGAC | 1017 |
| RB2-71 | GGTCGTACGCCTCGTTCTTGACAGCCGCGGCCCGTCGGGC | 1018 |
| RB2-72 | ACGAGGGGGGCACGATGCCAGCCCGACGGGCCGCGGCTGT | 1019 |
| RB3-71 | GGTCGTACGCCTCGTTCTTGACAGCCGCGCTCCGTCGGGC | 1020 |
| RB3-72 | ACGAGGGGGGCACGATGCCAGCCCGACGGAGCGCGGCTGT | 1021 |
| RB1-71 | GGTCGTACGCCTCGTTCTTGACAGCCGCGGCCCGTCGGGC | 1022 |
| RB1-72 | ACGAGGGGGGCACGATGCCAGCCCGACGGGCCGCGGCTGT | 1023 |
| RB4-82 | TGTCGACACGAGCGCGTGGAAGGTGACGGCCACGGAGGTT | 1024 |
| RB4-83 | TCCACGCGCTCGTGTCGACACAGTTTGGCCACACGGTCAA | 1025 |
| RB4-71 | GGTCGTACGCCTCGTTCTTGACAGCCGCGGCCCGTCGGGC | 1026 |
| RB4-72 | ACGAGGGGGGCACGATGCCAGCCCGACGGGCCGCGGCTGT | 1027 |
| RB5-71 | GGTCGTACGCCTCGTTCTTGACAGCCGCGGCCCGTCGGGC | 1028 |
| RB5-72 | ACGAGGGGGGCACGATGCCAGCCCGACGGGCCGCGGCTGT | 1029 |
| RB5-87 | AGCGCCGCCGTGGCTCTGGACGCCGTCAACTATCGCGATA | 1030 |
| RB5-88 | CCCAATCCACAGGGGGTGGTTATCGCGATAGTTGACGGCG | 1031 |
| RB6-16 | GGTGAAGCGGTCGATGAGGATCTTGAAGACAAGAGCGCTA | 1032 |
| RB6-17 | TCCTCATCGACCGCTTCACCGAAACGTACGATGCGGGCCT | 1033 |
| RB6-71 | GGTCGTACGCCTCGTTCTTGACAGCCGCGGCCCGTCGGGC | 1034 |
| RB6-72 | ACGAGGGGGGCACGATGCCAGCCCGACGGGCCGCGGCTGT | 1035 |
| RB6-87 | AGCGCCGCCGTGGCTCTGGACGCCGTCAACTATCGCGATA | 1036 |
| RB6-88 | CCCAATCCACAGGGGGTGGTTATCGCGATAGTTGACGGCG | 1037 |
| RB7-69 | CGCAACAGCGGCACTCCGCTGTCTGCGGTTCACCTGACGT | 1038 |
| RB7-70 | CAAGAACGAGGCGTACGACCACGTCAGGTGAACCGCAGAC | 1039 |
| RB7-71 | GGTCGTACGCCTCGTTCTTGACAGCCGCGGCCCGTCGGGC | 1040 |
| RB7-72 | ACGAGGGGGGCACGATGCCAGCCCGACGGGCCGCGGCTGT | 1041 |
| RB7-87 | AGCGCCGCCGTGGCTCTGGACGCCGTCAACTATCGCGATA | 1042 |
| RB7-88 | CCCAATCCACAGGGGGTGGTTATCGCGATAGTTGACGGCG | 1043 |
| RB8-71 | GGTCGTACGCCTCGTTCTTGACAGCCGCGCTCCGTCGGGC | 1044 |
| RB8-72 | ACGAGGGGGGCACGATGCCAGCCCGACGGAGCGCGGCTGT | 1045 |
| RB8-87 | AGCGCCGCCGTGGCTCTGGACGCCGTCAACTATCGCGATA | 1046 |
| RB8-88 | CCCAATCCACAGGGGGTGGTTATCGCGATAGTTGACGGCG | 1047 |
| RB9-71 | GGTCGTACGCCTCGTTCTTGACAGCCGCGCTCCGTCGGGC | 1048 |
| RB9-72 | ACGAGGGGGGCACGATGCCAGCCCGACGGAGCGCGGCTGT | 1049 |
| RB9-82 | TGTCGACACGAGCGCGTGGAAGGTGACGGCCACGGAGGTT | 1050 |
| RB9-83 | TCCACGCGCTCGTGTCGACACAGTTTGGCCACACGGTCAA | 1051 |
| RB10-16 | GGTGAAGCGGTCGATGAGGATCTTGAAGACAAGAGCGCTA | 1052 |
| RB10-17 | TCCTCATCGACCGCTTCACCGAAACGTACGATGCGGGCCT | 1053 |
| RB10-71 | GGTCGTACGCCTCGTTCTTGACAGCCGCGCTCCGTCGGGC | 1054 |
| RB10-72 | ACGAGGGGGGCACGATGCCAGCCCGACGGAGCGCGGCTGT | 1055 |
| RB10-87 | AGCGCCGCCGTGGCTCTGGACGCCGTCAACTATCGCGATA | 1056 |
| RB10-88 | CCCAATCCACAGGGGGTGGTTATCGCGATAGTTGACGGCG | 1057 |
| RB11-69 | CGCAACAGCGGCACTCCGCTGTCTGCGGTTCACCTGACGT | 1058 |
| RB11-70 | CAAGAACGAGGCGTACGACCACGTCAGGTGAACCGCAGAC | 1059 |
| RB11-71 | GGTCGTACGCCTCGTTCTTGACAGCCGCGCTCCGTCGGGC | 1060 |
| RB11-72 | ACGAGGGGGGCACGATGCCAGCCCGACGGAGCGCGGCTGT | 1061 |
| RB11-87 | AGCGCCGCCGTGGCTCTGGACGCCGTCAACTATCGCGATA | 1062 |
| RB11-88 | CCCAATCCACAGGGGGTGGTTATCGCGATAGTTGACGGCG | 1063 |
| RB12-16 | GGTGAAGCGGTCGATGAGGATCTTGAAGACAAGAGCGCTA | 1064 |
| RB12-17 | TCCTCATCGACCGCTTCACCGAAACGTACGATGCGGGCCT | 1065 |
| RB13-69 | CGCAACAGCGGCACTCCGCTGTCTGCGGTTCACCTGACGT | 1066 |
| RB13-70 | CAAGAACGAGGCGTACGACCACGTCAGGTGAACCGCAGAC | 1067 |
| RB14-71 | GGTCGTACGCCTCGTTCTTGACAGCCACGCTCCGTCGGGC | 1068 |
| RB14-72 | ACGAGGGGGGCACGATGCCAGCCCGACGGAGCGTGGCTGT | 1069 |
| RB15-87 | AGCGCCGCCGTGGCTCTGGACGCCGTCAACTATCGCGATA | 1070 |
| RB15-88 | CCCAATCCACAGGGGGTGGTTATCGCGATAGTTGACGGCG | 1071 |
| RB16-16 | GGTGAAGCGGTCGATGAGGATCTTGAAGACAAGAGCGCTA | 1072 |
| RB16-17 | TCCTCATCGACCGCTTCACCGAAACGTACGATGCGGGCCT | 1073 |
| RB16-87 | AGCGCCGCCGTGGCTCTGGACGCCGTCAACTATCGCGATA | 1074 |
| RB16-88 | CCCAATCCACAGGGGGTGGTTATCGCGATAGTTGACGGCG | 1075 |
| RB17-82 | TGTCGACACGAGCGCGTGGAAGGTGACGGCCACGGAGGTT | 1076 |
| RB17-83 | TCCACGCGCTCGTGTCGACACAGTTTGGCCACACGGTCAA | 1077 |
| RB17-87 | AGCGCCGCCGTGGCTCTGGACGCCGTCAACTATCGCGATA | 1078 |
| RB17-88 | CCCAATCCACAGGGGGTGGTTATCGCGATAGTTGACGGCG | 1079 |
| RB18-71 | GGTCGTACGCCTCGTTCTTGACAGCCATGGCCCGTCGGGC | 1080 |
| RB18-72 | ACGAGGGGGGCACGATGCCAGCCCGACGGGCCATGGCTGT | 1081 |
| RB18-87 | AGCGCCGCCGTGGCTCTGGACGCCGTCAACTATCGCGATA | 1082 |
| RB18-88 | CCCAATCCACAGGGGGTGGTTATCGCGATAGTTGACGGCG | 1083 |
| RB19-16 | GGTGAAGCGGTCGATGAGGATCTTGAAGACAAGAGCGCTA | 1084 |
| RB19-17 | TCCTCATCGACCGCTTCACCGAAACGTACGATGCGGGCCT | 1085 |
| RB19-71 | GGTCGTACGCCTCGTTCTTGACAGCCATGGCCCGTCGGGC | 1086 |
| RB19-72 | ACGAGGGGGGCACGATGCCAGCCCGACGGGCCATGGCTGT | 1087 |
| RB19-87 | AGCGCCGCCGTGGCTCTGGACGCCGTCAACTATCGCGATA | 1088 |
| RB19-88 | CCCAATCCACAGGGGGTGGTTATCGCGATAGTTGACGGCG | 1089 |
| RB20-16 | GGTGAAGCGGTCGATGAGGATCTTGAAGACAAGAGCGCTA | 1090 |
| RB20-17 | TCCTCATCGACCGCTTCACCGAAACGTACGATGCGGGCCT | 1091 |
| RB20-69 | CGCAACAGCGGCACTCCGCTGTCTGCGGTTCACCTGACGT | 1092 |
| RB20-70 | CAAGAACGAGGCGTACGACCACGTCAGGTGAACCGCAGAC | 1093 |
| RB20-71 | GGTCGTACGCCTCGTTCTTGACAGCCATGGCCCGTCGGGC | 1094 |
| RB20-72 | ACGAGGGGGGCACGATGCCAGCCCGACGGGCCATGGCTGT | 1095 |
| RB20-87 | AGCGCCGCCGTGGCTCTGGACGCCGTCAACTATCGCGATA | 1096 |
| RB20-88 | CCCAATCCACAGGGGGTGGTTATCGCGATAGTTGACGGCG | 1097 |

Variants were purified from large-scale fermentation, i.e., 100 ml or 500 ml fermentation, and PIs of thermal stability (Ts) and specific activities were determined. Specifically, specific activities were determined using different substrates, including DP2, DP3, DP4, DP5, DP6, DP7, cornstarch (CS), and liquefact (Liq). PIs are presented in Table 8. “N/D” in Table 8 stands for “not done.”

TABLE 8
PIs of representative combinatorial variants

| Variant | mutations | DP2 | DP3 | DP4 | DP5 | DP6 | DP7 | CS | Ts | Li [symbol] |
|---|---|---|---|---|---|---|---|---|---|---|
| ALL1 | I43Q/D44C/L417V/E503A/Q511H/A539R | 0.89 | 1.22 | 1.08 | 1.10 | 1.17 | 1.02 | 1.27 | 1.95 | 1. [symbol] |
| ALL2 | I43Q/L417V/E503A/Q511H/A539R | 1.13 | 1.50 | 1.24 | 1.19 | 1.32 | 1.24 | 1.52 | 1.84 | 1. [symbol] |
| ALL3 | I43Q/D44C/N61I/L417V/E503A/Q511H/A539R | 0.69 | 1.09 | 0.91 | 0.89 | 0.96 | 0.99 | 1.30 | 1.43 | 1. [symbol] |
| ALL4 | I43Q/N61I/L417V/E503A/Q511H/A539R | 0.89 | 1.40 | 1.15 | 1.15 | 1.25 | 1.12 | 1.52 | 1.79 | 1. [symbol] |
| ALL5 | I43R/L417V/E503A/Q511H/A539R | 1.10 | 1.50 | N/D | N/D | N/D | 1.04 | 1.17 | 2.37 | 1. [symbol] |
| ALL6 | I43R/N61I/L417V/E503A/Q511H/A539R | 0.93 | 1.52 | N/D | N/D | N/D | 1.37 | 1.53 | 1.84 | 2. [symbol] |
| ALL7 | I43R/L417R/E503A/A539R | 1.19 | 1.38 | 1.29 | 1.23 | 1.33 | 1.20 | 1.54 | 1.67 | 1. [symbol] |
| ALL8 | I43R/N61I/L417R/E503A/Q511H/A539R | 1.07 | 1.56 | N/D | N/D | N/D | 1.08 | 1.14 | 1.84 | 2. [symbol] |
| C1 | G73F/T430A/Q511H | 0.82 | 0.80 | 0.95 | 0.95 | 1.06 | 0.94 | 1.07 | 1.84 | 0. [symbol] |
| C2 | I43R/G73F/T430A | 0.96 | 0.97 | 1.14 | 1.17 | 1.28 | 1.07 | 1.25 | 1.91 | 1. [symbol] |
| C4 | G73F/T430A/E503V/Q511H | 0.97 | 0.80 | 1.10 | 1.08 | 1.17 | 1.04 | 1.06 | 2.57 | 1. [symbol] |
| C5 | D44C/G73F/N563K | 0.77 | 0.77 | 0.86 | 0.87 | 0.95 | 0.77 | 0.71 | 2.16 | 0. [symbol] |
| C7 | D44C/G73F/E503V/Q511H | 0.74 | 0.71 | 0.82 | 0.84 | 0.93 | 0.78 | 0.79 | 2.10 | 0. [symbol] |
| C8 | D44C/G73F/N563K | 0.78 | 0.76 | 0.81 | 0.91 | 0.94 | 0.79 | 1.05 | 2.28 | N [symbol] |
| C9 | D44C/G73F/L417R/N563K | 0.82 | 0.86 | 0.96 | 1.07 | 1.08 | 0.96 | 1.00 | 2.57 | 1. [symbol] |
| C11 | D44C/G73F/N563K | 0.74 | 0.68 | 0.81 | 0.92 | 0.90 | 0.76 | 1.04 | 2.42 | N [symbol] |
| C12 | I43R/T430A | 1.02 | 1.06 | 1.21 | 1.21 | 1.32 | 1.03 | 1.35 | 1.60 | 1. [symbol] |
| C13 | I43Q/T430A | 0.96 | 0.94 | 1.13 | 1.14 | 1.24 | 1.04 | 1.29 | 1.10 | 1. [symbol] |
| C14 | I43Q/T430A/Q511H | 1.08 | 1.11 | 1.13 | 1.14 | 1.23 | 1.13 | 1.34 | 1.33 | 1. [symbol] |
| C15 | D44C/L417R/N563K | 0.84 | 0.88 | 0.95 | 1.07 | 1.08 | 0.85 | 1.09 | 2.30 | 0. [symbol] |
| CS1 | L417V/T430A/A431L/Q511H/A535R/A539R/N563I | 0.78 | 1.19 | 1.35 | 1.29 | 1.42 | 1.28 | 1.44 | 1.56 | 2. [symbol] |
| CS2 | L417V/T430A/A431Q/Q511H/A535R/A539R/N563I | 0.96 | 1.39 | 1.46 | 1.42 | 1.52 | 1.60 | 1.77 | 1.56 | 2. [symbol] |
| CS3 | L417V/T430A/Q511H/A535R/N563I | 1.00 | 1.17 | N/D | N/D | N/D | 0.98 | 1.40 | 1.69 | 1. [symbol] |
| CS4 | L417V/T430A/Q511H/A539R/N563I | 1.11 | 1.36 | N/D | N/D | N/D | 1.21 | 1.53 | 1.95 | 1. [symbol] |
| LQ1 | G294C/L417R/A431L | 1.10 | 1.47 | 1.18 | 1.18 | 1.35 | 1.10 | 0.98 | 1.04 | 1. [symbol] |
| LQ2 | G294C/L417V/A431Q | 1.15 | 1.56 | 1.32 | 1.30 | 1.46 | 1.22 | 1.08 | 1.02 | 2. [symbol] |
| LQ3 | G294C/L417V/A431L/Q511H | 0.99 | 1.41 | N/D | N/D | N/D | N/D | 1.21 | 1.29 | 2. [symbol] |
| LQ4 | G294C/L417R/A431Q/Q511H | 1.20 | 1.49 | 1.11 | 1.13 | 1.27 | 1.08 | 1.19 | 1.43 | 1. [symbol] |
| LQ5 | L417R/A431L/Q511H | N/D | N/D | N/D | N/D | N/D | 1.02 | 1.21 | 1.56 | 1. [symbol] |
| LQ6 | L417V/A431Q/Q511H | 0.94 | 1.30 | 1.20 | 1.18 | 1.28 | 1.25 | 1.33 | 1.35 | 2. [symbol] |
| RB1 | I43Q/T430A/Q511H/N61I | 0.90 | 1.13 | 1.00 | 1.03 | 1.13 | 1.08 | 1.15 | 1.27 | 1. [symbol] |
| RB2 | I43Q/T430A/Q511H/L417V | 1.12 | 1.34 | N/D | N/D | N/D | 1.09 | 1.38 | 1.42 | 1. [symbol] |
| RB3 | I43Q/T430A/Q511H/A431L | 0.87 | 1.05 | 1.03 | 1.05 | 1.15 | 1.10 | 1.25 | 1.29 | 1. [symbol] |
| RB4 | I43Q/T430A/Q511H/E503A | 0.99 | 1.00 | 1.05 | 1.04 | 1.13 | 1.09 | 1.29 | 1.47 | 1. [symbol] |
| RB5 | I43Q/T430A/Q511H/A539R | 0.98 | 1.22 | 1.06 | 1.02 | 1.11 | 1.10 | 1.45 | 1.47 | 1. [symbol] |
| RB6 | I43Q/T430A/Q511H/N61I/A539R | 0.75 | 1.09 | 1.09 | 1.05 | 1.14 | 1.23 | N/D | 0.94 | N [symbol] |
| RB7 | I43Q/T430A/Q511H/L417V/A539R | 1.20 | 1.62 | N/D | N/D | N/D | 1.29 | 1.44 | 1.85 | 2. [symbol] |
| RB8 | I43Q/T430A/Q511H/A431L/A539R | 0.90 | 1.38 | N/D | N/D | N/D | 1.25 | 1.38 | 1.59 | 2. [symbol] |
| RB9 | I43Q/T430A/Q511H/A431L/E503A | 0.79 | 0.82 | 0.95 | 0.92 | 1.02 | 1.03 | 1.39 | 1.69 | 1. [symbol] |
| RB10 | I43Q/T430A/Q511H/N61I/A539R/A431L | 0.68 | 1.27 | N/D | N/D | N/D | 0.87 | 1.47 | 1.29 | 1. [symbol] |
| RB11 | I43Q/T430A/Q511H/L417V/A539R/A431L | 0.89 | 1.50 | N/D | N/D | N/D | 1.05 | 1.36 | 1.58 | 2. [symbol] |
| RB12 | I43Q/Q511H/N61I | 0.75 | 0.80 | 0.91 | 0.91 | 1.00 | 0.82 | 1.25 | 1.37 | 1. [symbol] |
| RB13 | I43Q/Q511H/L417V | 0.97 | 0.94 | 0.96 | 0.93 | 1.05 | 0.94 | 1.38 | 1.50 | 1. [symbol] |
| RB14 | I43Q/Q511H/A431L | 0.73 | 0.75 | 0.89 | 0.88 | 0.94 | 0.89 | 1.31 | 1.37 | 1. [symbol] |
| RB15 | I43Q/Q511H/A539R | 0.90 | 0.88 | 1.03 | 0.99 | 1.07 | 1.05 | 1.42 | 1.52 | 1. [symbol] |
| RB16 | I43Q/Q511H/A539R/N61I | 0.78 | 0.96 | 1.22 | 1.18 | 1.26 | 0.94 | 1.43 | 1.60 | 1. [symbol] |
| RB17 | I43Q/Q511H/A539R/E503A | 1.02 | 1.15 | 1.16 | 1.12 | 1.21 | 1.20 | 1.38 | 1.68 | 1. [symbol] |
| RB18 | I43Q/Q511H/A539R/T430M | 0.81 | 0.84 | 1.03 | 0.99 | 1.08 | 0.54 | 1.44 | 1.64 | 1. [symbol] |
| RB19 | I43Q/Q511H/A539R/T430M/N61I | 0.77 | 1.21 | N/D | N/D | N/D | 1.07 | 1.50 | 1.47 | 1. [symbol] |
| RB20 | I43Q/Q511H/A539R/T430M/N61I/L417V | 0.73 | 1.35 | N/D | N/D | N/D | 0.96 | 1.55 | 1.79 | 1. [symbol] |
| TS1 | I43R/T430A/E503V/A535R/N563K | 1.09 | 0.95 | 1.13 | 1.09 | 1.17 | 1.19 | N/D | 2.13 | N [symbol] |
| TS2 | D44R/E503A/Q511H/N563I | 0.80 | 0.70 | 0.88 | 0.89 | 0.96 | 0.85 | 1.14 | 1.53 | 1. [symbol] |
| TS3 | E503A/N563I | 0.93 | 0.73 | 0.91 | 0.88 | 0.96 | 0.92 | 1.17 | 1.39 | 1. [symbol] |
| TS4 | I43R/T430A/E503A/Q511H/N563K | 0.92 | 0.77 | 0.95 | 0.89 | 1.02 | 1.06 | 1.33 | 1.66 | 1. [symbol] |
| TS5 | D44R/T430A/Q511H/A535R | 0.74 | 0.68 | 0.83 | 0.83 | 0.91 | 0.91 | 0.95 | 1.59 | 1. [symbol] |
| Var1 | L417V/A431L/A539R | 0.69 | 1.06 | N/D | 1.12 | N/D | 1.16 | 1.26 | 1.18 | 1. [symbol] |
| Var2 | L417V/A431L/A539R/I43Q | 0.69 | 1.13 | N/D | 1.15 | N/D | 1.18 | 1.40 | 1.12 | 1. [symbol] |
| Var3 | L417V/A431L/A539R/N61I | 0.43 | 0.91 | N/D | 0.99 | N/D | 1.06 | 1.29 | 1.14 | 1. [symbol] |
| Var4 | L417V/A431L/A539R/A535R | 0.64 | 0.97 | N/D | 1.03 | N/D | 1.07 | 1.22 | 1.49 | 1. [symbol] |
| Var5 | L417V/A431L/A539R/I43Q/N61I | 0.38 | 0.86 | N/D | 0.94 | N/D | 1.04 | 1.25 | 1.07 | 1. [symbol] |
| Var6 | L417V/A431L/A539R/N61I/A535R | 0.32 | 0.72 | N/D | 0.82 | N/D | 0.90 | 1.16 | 1.86 | 1. [symbol] |
| Var7 | L417V/A431L/A539R/A535R/I43Q | 0.65 | 0.99 | N/D | 1.03 | N/D | 1.10 | 1.22 | 1.29 | 1. [symbol] |
| Var8 | L417V/A431L/A539R/I43Q/N61I/A535R | 0.32 | 0.73 | N/D | 0.80 | N/D | 0.94 | 1.11 | 1.08 | 1. [symbol] |
| Var9 | L417V/A431L/A539R/I43Q/N61I/A535R/T430A | 0.25 | 0.70 | N/D | 0.71 | N/D | 0.84 | 0.99 | 1.13 | 1. [symbol] |
| Var10 | L417V/T430A/A431L/Q511H/A535R/A539R/N563I/I43Q | 0.53 | 0.98 | N/D | 0.97 | N/D | 1.06 | 1.08 | 1.86 | 1. [symbol] |
| Var11 | L417V/T430A/A431L/Q511H/A535R/A539R/N563I/N61I | 0.38 | 0.80 | N/D | 0.86 | N/D | 0.95 | 1.16 | 1.51 | 1. [symbol] |
| Var12 | L417V/T430A/A431L/Q511H/A535R/A539R/N563I/I43Q/ | 0.31 | 0.78 | N/D | 0.84 | N/D | 0.94 | 1.17 | 1.81 | 1. [symbol] |
| | N61I | | | | | | | | | |
| Var13 | L417V/A431L/A539R/I43R | 0.67 | 1.08 | N/D | 1.12 | N/D | 1.18 | 1.32 | 1.83 | 1. [symbol] |
| Var14 | L417V/A431L/A539R/I43R/N61I | 0.43 | 0.99 | N/D | 1.01 | N/D | 1.09 | 1.24 | 1.70 | 1. [symbol] |
| Var15 | L417V/A431L/A539R/I43R/N61I/A535R/T430A | 0.39 | 0.94 | N/D | 0.96 | N/D | 1.09 | 1.34 | 1.96 | 1. [symbol] |
| Var16 | L417R/A431L/A539R | 0.70 | 1.01 | N/D | 1.03 | N/D | 1.11 | 1.23 | 1.33 | 1. [symbol] |
| Var17 | L417G/A431L/A539R | 0.70 | 1.15 | N/D | 1.14 | N/D | 1.19 | 1.20 | 1.23 | 1. [symbol] |
| Var18 | G73F/E503V/N563K/L417R/A539R | 0.93 | 1.14 | N/D | 1.12 | N/D | 1.11 | 1.08 | 2.71 | 0. [symbol] |
| Var19 | G73F/E503V/N563K/I43R/L417R/A539R | 1.24 | 1.47 | N/D | 1.43 | N/D | 1.33 | 1.38 | 2.58 | 1. [symbol] |
| Var20 | G73F/E503V/N563K/I43R/Q511H | 0.94 | 0.95 | N/D | 0.95 | N/D | 0.97 | 1.02 | 2.47 | 0. [symbol] |

[symbol]

indicates data missing or illegible when filed

### Example 9

### Crystal Structure of TrGA

The complete three dimensional structure of Trichoderma reesei (Hypocrea jecorina) glucoamylase (TrGA) was determined at 1.9 Å resolution. Table 9 shows the coordinates for the Trichoderma glucoamylase crystal structure. TrGA was crystallized in an intact form containing 599 residues and all post-translational modifications that would normally occur in the natural host. The crystal structure was produced and analyzed as follows:

For protein expression and purification, the gene encoding H. jecorina GA was cloned and expressed according to the protocols described in the U.S. Pat. No. 7,413,887.

The TrGA protein material used for all crystallization experiments was initially purified in one step by anion exchange chromatography as follows: concentrated culture supernatants of expressed TrGA, consisting of 180 mg/ml total protein, were prepared by diluting sample 1:10 in a 25 mM Tris-HCl, pH 8.0 buffer. A HiPrep 16/10 Q Sepharose FF column (GE Helthcare) was employed for the anion exchange purification. The HiPrep column was equilibrated with 4 column volumes (CV) starting buffer (25 mM Tris-HCl, pH 8.0) followed by application of 10 ml of the diluted protein sample. An 8 CV linear gradient of 0 to 140 mM NaCl in the running buffer (25 mM Tris-HCl, pH 8.0) was applied to elute bound protein. Bound TrGA eluted from the HiPrep Q sepharose column at a salt concentration of approximately 80 mM NaCl. Fractions containing pure TrGA protein were pooled and concentrated to 50 mg/ml using a 25 ml Vivaspin centrifugal concentration tube (Viva Science) with a molecular weight cutoff (MWCO) of 10 kD. Purified and concentrated TrGA material was buffer exchanged using a DG-10 desalting column (Bio-Rad) equilibrated with 50 mM sodium acetate buffer, pH 4.3. Protein concentrations were determined by measuring the absorbance at 280 nm. The initially purified and concentrated TrGA protein stock was thereafter stored at −20° C.

Two additional purification steps, on additional anion exchange purification, and a size exclusion purification, were introduced to enhance the crystability of the TrGA protein material. These two additional purification steps were performed as follows. In the first anion exchange purification step a 10 ml MonoQ column (GE Helthcare) was employed. A sample of 1 ml of the initially purified and frozen TrGA material (50 mg protein) was thawed and the buffer was changed to 20 mM Tris-HCl, pH 8.0, by repeated dilution of the sample to 6 ml in the new buffer, followed by a concentration of the sample again to 0.5 ml using a 6 ml 5 kD MWCO concentration tube. The TrGA sample was diluted after the last concentration step in distilled water until a conductivity of the protein sample was reached that corresponded to the conductivity of the starting buffer of the anion purification, i.e. 25 mM Tris-HCl, pH 8.0. The MonoQ column was first equilibrated with 4 column volumes (CV) starting buffer, followed by application of the diluted protein sample to the column. Bound protein was eluted from the MonoQ column by two different gradients. In the first a 4 CV linear pH gradient was applied where the pH of the starting buffer was decreased from 8.0 to 6.0. In the second gradient an 8 CV long salt gradient was applied in which the salt concentration was increased from 0 to 350 mM NaCl in the running buffer (25 mM Tris-HCl, pH 6.0). Bound TrGA was found to elute from the column during the second salt gradient at an approximate NaCl concentration of 150 mM. Fractions containing TrGA were pooled and concentrated to 2 ml using a 6 ml 5 kD MWCO Vivaspin concentration tube. The concentrated TrGA sample was thereafter applied to a Superdex 200 16/60 size exclusion column (GE Helthcare) equilibrated with 4 CV of 20 mM Tris-Cl, pH 8.0, and 50 mM NaCl, which also was used as running buffer. Fractions from the main elution peak after the size exclusion purification were pooled and concentrated to an approximate protein concentration of 7.5 mg/ml using a 6 ml 5 kD MWCO Vivaspin concentration tube.

For protein crystallization, the protein sample that was used to find the initial TrGA crystallization conditions was a sample of the TrGA material that was purified once by anion exchange purification and thereafter stored at −20° C. The TrGA protein sample was thawed and diluted with 50 mM sodium acetate buffer, pH 4.3, to approximately 12 mg/ml, prior to the initial crystallization experiments. The orthorhombic X-ray dataset, was used to solve the TrGA structure by molecular replacement (MR), and the high-resolution orthorhombic dataset, used for the final orthorhombic space group TrGA structure model. The orthorhombic TrGA crystals were found to grow in solution consisting of 25% PEG 3350, 0.20M ammonium acetate, 0.10M Bis-Tris pH 5.5 (reservoir solution), using the vapor-diffusion method with hanging drops (McPherson 1982), at 20° C. Crystallization drops were prepared by mixing equal amounts of protein solution (12 mg/ml) and reservoir solution to a final volume of 10 μl. The TrGA crystals were found to belong to the orthorhombic space group P212121 with approximate cell dimensions: a=52.2 Å, b=99.2 Å, c=121.2 Å, and have a calculated Vₘ of 2.3 (Matthews, B. W. (1968) J. Mol. Biol. 33: 491-497) with one molecule in the asymmetric unit.

For X-ray data collection, the two orthorhombic TrGA datasets were collected from single crystals mounted in sealed capillary tubes, at room temperature. The initial lo-resolution orthorhombic TrGA X-ray dataset, used to solve the structure by molecular replacement methods (MR), was collected on a home X-ray source, an MSC/Rigaku (Molecular Structures Corp., The Woodlands, Tex.) Raxis IV++ image plate detector with focusing mirrors using Cu Kα radiation from a Rigaku RU200 rotating anode generator. This dataset was processed, scaled, and averaged using the d*trek software provided by MSC/Rigaku. The C centered monoclinic dataset was collected from a single frozen TrGA crystal at 100K, equilibrated in a cryo-protective agent comprised of 25% PEG 3350, 15% Glycerol 50 mM CaCl₂ and 0.1 M Bis-Tris pH 5.5 as cryoprotectant, mounted in rayon-fiber loops, and plunge frozen in liquid nitrogen prior to transportation to the synchrotron. The high-resolution orthorhombic (1.9 Å) data set and the C centric monoclinic dataset (1.8 Å) were both collected at a synchrotron source, beam line 911:5 at MAX LAB in Lund, Sweden. Both datasets that were collected at a synchrotron source were processed with MOSFLM, and scaled with program SCALA included in the CCP4 program package (Collaborative Computational Project Number 4 1994). All subsequent data processing was performed using the CCP4 program package (Collaborative Computational Project Number 4 1994), unless otherwise stated. A set of 5% of the reflections from each data set was set aside and used for monitoring the R-free (Brunger, A (1992) Nature, 355:472-475).

The TrGA structure was initially solved by MR with the automatic replacement program MOLREP (Collaborative Computational Project Number 4 1994), included in the CCP4 program package, using the initial lo-resolution orthorhombic dataset, and using the coordinates of Aspergillus. awamori GA (AaGA) variant X100 (pdb entry 1GLM (Aleshin et al. (1994) J. Mol. Boil. 238: 575-591) as search model. The A. awamori GA search model was edited to remove all glycosylation moieties attached to the protein molecule as N- and O-glycosylations, and all solvent molecules before carrying out the MR experiments. All reflections between 36.8 and 2.8 Å resolution, from the initial lo resolution TrGA dataset, was used for the MR solution. The MR program found a single rotation function solution, with a maxima of 11.1σ above background, the next highest maxima was 3.8a above the background. The translation function solution gave an R-factor of 48.7% and had a contrast factor of 17.4. The MR solution was refined for 10 cycles of restrained least squares refinement using the program Refmac 5.0 (Murshudov et al (1997) Acta Crystallogr. D53: 240-255). This lowered the crystallographic R-factor to 31.1% while the R-free value dropped from 42.2% to 41.1%.

The refined MR solution model was used to calculate an initial density map from the lo-resolution orthorhombic TrGA dataset. Electron density for a disulfide bridge between residues 19 and 26 of TrGA, a disulfide bridge not present in the A. awamori variant X100 structure model, could readily be identified in this electron density map. This was taken as an indication that the electron density map was of sufficient quality to be used to build a structure model of TrGA from its amino acid sequence. The initial TrGA structure model, based on the lo-resolution dataset, was refined with alternating cycles of model building using Coot (Emsley and Cowtan, (2004) Acta Crystallogr. D boil Crystallogr. 60: 2126-2132), and maximum likelihood refinement using Refmac 5.0.

The resolution of the initial TrGA structure model was extended to the resolution of the high-resolution orthorhombic dataset (1.9 Å) by refining the initial TrGA structure model against the high-resolution dataset for 10 cycles of restrained refinement using the program Refmac 5.0. Most water molecules in the structure models were located automatically by using the water picking protocols in the refinement programs, and then manually selected or discarded by inspection by eye. All structural comparisons were made with either Coot (Emsley and Cowtan (2004) supra) or O (Jones et al. (1991) Acta Crystallogr. A47: 110-119), and figures were prepared with PyMOL (Delano W. L. (2002) The PyMOL Molecular Graphics System. Palo Alto, Calif., USA; Delano Scientific).

From these results, it can be seen that the TrGA catalytic core segment followed the same (α/α)₆-barrel topology described by Aleshin et al. 1992 for the AaGA, consisting of a double barrel of alpha helices with the C-terminal of the outer helix leading into the N-terminus of an inner helix. It was possible to identify key differences in the electron density such as the disulfide bridge between residues 19 and 26 and an insertion (residues 257-260) relative to AaGA. The segment comprising 80-100 also underwent extensive model rebuilding. One major glycosylation site was identified at Asn 171, which had up to four glycoside moieties attached. A similar glycosylation site was identified in AaGA. Additionally, the catalytic core containing three cis-peptides between residues 22-23, 44-45 and 122-123 were conserved between TrGA and AaGA. Overall, there was an rms variation of 0.535 Å between 409 out of 453 Cα atoms when comparing the coordinates of the catalytic cores of TrGA and AaGA.

TABLE 9
X-ray coordinates of TrGA

| | | | | | | |
|---|---|---|---|---|---|---|
| CRYST1 | 52.185 | 99.232 | 121.240 | 90.00 | 90.00 | 90.00 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.019163 | −0.000001 | −0.000001 | 0.00000 |
| SCALE2 | 0.000000 | 0.010077 | 0.000000 | 0.00000 |
| SCALE3 | 0.000000 | 0.000000 | 0.008248 | 0.00000 |

| | | | | | | | | | | |
|---|---|---|---|---|---|---|---|---|---|---|
| ATOM | 1 | N | SER | A | 1 | −30.485 | 30.567 | −21.185 | 1.00 | 37.11 |
| ATOM | 2 | CA | SER | A | 1 | −30.568 | 29.350 | −20.326 | 1.00 | 37.00 |
| ATOM | 3 | CB | SER | A | 1 | −31.953 | 28.707 | −20.424 | 1.00 | 37.27 |
| ATOM | 4 | OG | SER | A | 1 | −32.137 | 28.089 | −21.695 | 1.00 | 40.11 |
| ATOM | 5 | C | SER | A | 1 | −29.519 | 28.345 | −20.772 | 1.00 | 35.91 |
| ATOM | 6 | O | SER | A | 1 | −29.043 | 28.415 | −21.911 | 1.00 | 35.46 |
| ATOM | 7 | N | VAL | A | 2 | −29.170 | 27.425 | −19.867 | 1.00 | 34.51 |
| ATOM | 8 | CA | VAL | A | 2 | −28.302 | 26.293 | −20.179 | 1.00 | 33.56 |
| ATOM | 9 | CB | VAL | A | 2 | −28.142 | 25.339 | −18.955 | 1.00 | 33.84 |
| ATOM | 10 | CG1 | VAL | A | 2 | −27.349 | 24.103 | −19.316 | 1.00 | 34.20 |
| ATOM | 11 | CG2 | VAL | A | 2 | −27.468 | 26.057 | −17.827 | 1.00 | 34.79 |
| ATOM | 12 | C | VAL | A | 2 | −28.846 | 25.506 | −21.363 | 1.00 | 32.48 |
| ATOM | 13 | O | VAL | A | 2 | −28.086 | 25.109 | −22.245 | 1.00 | 31.10 |
| ATOM | 14 | N | ASP | A | 3 | −30.160 | 25.286 | −21.381 | 1.00 | 31.43 |
| ATOM | 15 | CA | ASP | A | 3 | −30.791 | 24.530 | −22.457 | 1.00 | 31.38 |
| ATOM | 16 | CB | ASP | A | 3 | −32.283 | 24.323 | −22.190 | 1.00 | 32.17 |
| ATOM | 17 | CG | ASP | A | 3 | −32.522 | 23.492 | −20.943 | 1.00 | 35.28 |
| ATOM | 18 | OD1 | ASP | A | 3 | −32.413 | 22.251 | −21.028 | 1.00 | 36.80 |
| ATOM | 19 | OD2 | ASP | A | 3 | −32.786 | 24.092 | −19.870 | 1.00 | 40.63 |
| ATOM | 20 | C | ASP | A | 3 | −30.556 | 25.153 | −23.818 | 1.00 | 30.59 |
| ATOM | 21 | O | ASP | A | 3 | −30.282 | 24.446 | −24.778 | 1.00 | 30.19 |
| ATOM | 22 | N | ASP | A | 4 | −30.644 | 26.477 | −23.875 | 1.00 | 29.89 |
| ATOM | 23 | CA | ASP | A | 4 | −30.369 | 27.244 | −25.083 | 1.00 | 29.99 |
| ATOM | 24 | CB | ASP | A | 4 | −30.601 | 28.731 | −24.822 | 1.00 | 31.12 |
| ATOM | 25 | CG | ASP | A | 4 | −32.088 | 29.121 | −24.785 | 1.00 | 34.16 |
| ATOM | 26 | OD1 | ASP | A | 4 | −32.991 | 28.260 | −24.925 | 1.00 | 36.06 |
| ATOM | 27 | OD2 | ASP | A | 4 | −32.340 | 30.332 | −24.608 | 1.00 | 39.96 |
| ATOM | 28 | C | ASP | A | 4 | −28.925 | 27.049 | −25.579 | 1.00 | 28.65 |
| ATOM | 29 | O | ASP | A | 4 | −28.697 | 26.881 | −26.770 | 1.00 | 28.51 |
| ATOM | 30 | N | PHE | A | 5 | −27.961 | 27.096 | −24.660 | 1.00 | 26.74 |
| ATOM | 31 | CA | PHE | A | 5 | −26.553 | 26.860 | −24.994 | 1.00 | 25.21 |
| ATOM | 32 | CB | PHE | A | 5 | −25.666 | 27.110 | −23.764 | 1.00 | 25.59 |
| ATOM | 33 | CG | PHE | A | 5 | −24.244 | 26.646 | −23.931 | 1.00 | 26.03 |
| ATOM | 34 | CD1 | PHE | A | 5 | −23.395 | 27.259 | −24.854 | 1.00 | 27.29 |
| ATOM | 35 | CE1 | PHE | A | 5 | −22.063 | 26.823 | −25.009 | 1.00 | 27.33 |
| ATOM | 36 | CZ | PHE | A | 5 | −21.593 | 25.783 | −24.228 | 1.00 | 26.77 |
| ATOM | 37 | CE2 | PHE | A | 5 | −22.425 | 25.181 | −23.286 | 1.00 | 28.42 |
| ATOM | 38 | CD2 | PHE | A | 5 | −23.749 | 25.617 | −23.144 | 1.00 | 28.42 |
| ATOM | 39 | C | PHE | A | 5 | −26.352 | 25.438 | −25.539 | 1.00 | 24.23 |
| ATOM | 40 | O | PHE | A | 5 | −25.659 | 25.244 | −26.544 | 1.00 | 23.56 |
| ATOM | 41 | N | ILE | A | 6 | −26.974 | 24.458 | −24.892 | 1.00 | 22.71 |
| ATOM | 42 | CA | ILE | A | 6 | −26.835 | 23.065 | −25.312 | 1.00 | 22.36 |
| ATOM | 43 | CB | ILE | A | 6 | −27.491 | 22.106 | −24.299 | 1.00 | 21.86 |
| ATOM | 44 | CG1 | ILE | A | 6 | −26.744 | 22.181 | −22.956 | 1.00 | 22.27 |
| ATOM | 45 | CD1 | ILE | A | 6 | −27.384 | 21.347 | −21.834 | 1.00 | 22.36 |
| ATOM | 46 | CG2 | ILE | A | 6 | −27.571 | 20.669 | −24.848 | 1.00 | 21.69 |
| ATOM | 47 | C | ILE | A | 6 | −27.388 | 22.855 | −26.723 | 1.00 | 22.84 |
| ATOM | 48 | O | ILE | A | 6 | −26.753 | 22.216 | −27.573 | 1.00 | 21.76 |
| ATOM | 49 | N | SER | A | 7 | −28.556 | 23.420 | −26.996 | 1.00 | 23.10 |
| ATOM | 50 | CA | SER | A | 7 | −29.146 | 23.175 | −28.309 | 1.00 | 23.90 |
| ATOM | 51 | CB | SER | A | 7 | −30.627 | 23.570 | −28.320 | 1.00 | 25.04 |
| ATOM | 52 | OG | SER | A | 7 | −30.717 | 24.982 | −28.282 | 1.00 | 30.08 |
| ATOM | 53 | C | SER | A | 7 | −28.340 | 23.874 | −29.422 | 1.00 | 22.78 |
| ATOM | 54 | O | SER | A | 7 | −28.186 | 23.337 | −30.508 | 1.00 | 22.94 |
| ATOM | 55 | N | THR | A | 8 | −27.800 | 25.053 | −29.140 | 1.00 | 22.50 |
| ATOM | 56 | CA | THR | A | 8 | −26.984 | 25.780 | −30.115 | 1.00 | 23.05 |
| ATOM | 57 | CB | THR | A | 8 | −26.834 | 27.247 | −29.698 | 1.00 | 23.65 |
| ATOM | 58 | OG1 | THR | A | 8 | −28.138 | 27.839 | −29.700 | 1.00 | 27.60 |
| ATOM | 59 | CG2 | THR | A | 8 | −25.939 | 28.018 | −30.660 | 1.00 | 26.76 |
| ATOM | 60 | C | THR | A | 8 | −25.601 | 25.159 | −30.307 | 1.00 | 21.46 |
| ATOM | 61 | O | THR | A | 8 | −25.109 | 25.051 | −31.437 | 1.00 | 21.38 |
| ATOM | 62 | N | GLU | A | 9 | −24.978 | 24.768 | −29.200 | 1.00 | 19.11 |
| ATOM | 63 | CA | GLU | A | 9 | −23.596 | 24.269 | −29.243 | 1.00 | 18.01 |
| ATOM | 64 | CB | GLU | A | 9 | −22.959 | 24.334 | −27.847 | 1.00 | 17.76 |
| ATOM | 65 | CG | GLU | A | 9 | −21.449 | 23.945 | −27.794 | 1.00 | 17.71 |
| ATOM | 66 | CD | GLU | A | 9 | −20.536 | 24.892 | −28.609 | 1.00 | 20.86 |
| ATOM | 67 | OE1 | GLU | A | 9 | −20.949 | 26.010 | −28.971 | 1.00 | 19.89 |
| ATOM | 68 | OE2 | GLU | A | 9 | −19.389 | 24.500 | −28.909 | 1.00 | 19.22 |
| ATOM | 69 | C | GLU | A | 9 | −23.462 | 22.846 | −29.784 | 1.00 | 17.77 |
| ATOM | 70 | O | GLU | A | 9 | −22.423 | 22.505 | −30.368 | 1.00 | 18.05 |
| ATOM | 71 | N | THR | A | 10 | −24.485 | 22.020 | −29.593 | 1.00 | 15.87 |
| ATOM | 72 | CA | THR | A | 10 | −24.404 | 20.609 | −29.958 | 1.00 | 17.31 |
| ATOM | 73 | CB | THR | A | 10 | −25.677 | 19.823 | −29.525 | 1.00 | 17.46 |
| ATOM | 74 | OG1 | THR | A | 10 | −25.768 | 19.860 | −28.090 | 1.00 | 17.46 |
| ATOM | 75 | CG2 | THR | A | 10 | −25.616 | 18.374 | −30.037 | 1.00 | 18.42 |
| ATOM | 76 | C | THR | A | 10 | −24.026 | 20.346 | −31.430 | 1.00 | 17.40 |
| ATOM | 77 | O | THR | A | 10 | −23.073 | 19.615 | −31.682 | 1.00 | 17.22 |
| ATOM | 78 | N | PRO | A | 11 | −24.764 | 20.934 | −32.412 | 1.00 | 18.30 |
| ATOM | 79 | CA | PRO | A | 11 | −24.346 | 20.649 | −33.798 | 1.00 | 18.11 |
| ATOM | 80 | CB | PRO | A | 11 | −25.440 | 21.317 | −34.662 | 1.00 | 18.57 |
| ATOM | 81 | CG | PRO | A | 11 | −26.094 | 22.310 | −33.771 | 1.00 | 19.16 |
| ATOM | 82 | CD | PRO | A | 11 | −25.975 | 21.779 | −32.361 | 1.00 | 18.54 |
| ATOM | 83 | C | PRO | A | 11 | −22.963 | 21.231 | −34.142 | 1.00 | 17.81 |
| ATOM | 84 | O | PRO | A | 11 | −22.241 | 20.655 | −34.964 | 1.00 | 17.74 |
| ATOM | 85 | N | ILE | A | 12 | −22.601 | 22.353 | −33.520 | 1.00 | 16.85 |
| ATOM | 86 | CA | ILE | A | 12 | −21.279 | 22.936 | −33.731 | 1.00 | 16.66 |
| ATOM | 87 | CB | ILE | A | 12 | −21.161 | 24.319 | −33.112 | 1.00 | 17.25 |
| ATOM | 88 | CG1 | ILE | A | 12 | −22.194 | 25.267 | −33.751 | 1.00 | 19.25 |
| ATOM | 89 | CD1 | ILE | A | 12 | −22.289 | 26.635 | −33.101 | 1.00 | 21.45 |
| ATOM | 90 | CG2 | ILE | A | 12 | −19.714 | 24.855 | −33.270 | 1.00 | 18.75 |
| ATOM | 91 | C | ILE | A | 12 | −20.170 | 22.023 | −33.178 | 1.00 | 16.30 |
| ATOM | 92 | O | ILE | A | 12 | −19.155 | 21.798 | −33.848 | 1.00 | 14.64 |
| ATOM | 93 | N | ALA | A | 13 | −20.360 | 21.527 | −31.951 | 1.00 | 15.28 |
| ATOM | 94 | CA | ALA | A | 13 | −19.375 | 20.627 | −31.304 | 1.00 | 15.19 |
| ATOM | 95 | CB | ALA | A | 13 | −19.788 | 20.332 | −29.883 | 1.00 | 15.31 |
| ATOM | 96 | C | ALA | A | 13 | −19.204 | 19.326 | −32.092 | 1.00 | 15.37 |
| ATOM | 97 | O | ALA | A | 13 | −18.083 | 18.834 | −32.297 | 1.00 | 13.56 |
| ATOM | 98 | N | LEU | A | 14 | −20.320 | 18.743 | −32.531 | 1.00 | 15.13 |
| ATOM | 99 | CA | LEU | A | 14 | −20.225 | 17.503 | −33.285 | 1.00 | 16.06 |
| ATOM | 100 | CB | LEU | A | 14 | −21.630 | 16.921 | −33.510 | 1.00 | 17.33 |
| ATOM | 101 | CG | LEU | A | 14 | −21.689 | 15.563 | −34.212 | 1.00 | 20.02 |
| ATOM | 102 | CD1 | LEU | A | 14 | −20.946 | 14.460 | −33.471 | 1.00 | 23.09 |
| ATOM | 103 | CD2 | LEU | A | 14 | −23.150 | 15.225 | −34.390 | 1.00 | 21.86 |
| ATOM | 104 | C | LEU | A | 14 | −19.506 | 17.749 | −34.623 | 1.00 | 15.61 |
| ATOM | 105 | O | LEU | A | 14 | −18.651 | 16.947 | −35.039 | 1.00 | 14.82 |
| ATOM | 106 | N | ASN | A | 15 | −19.853 | 18.852 | −35.285 | 1.00 | 15.30 |
| ATOM | 107 | CA | ASN | A | 15 | −19.236 | 19.228 | −36.567 | 1.00 | 16.34 |
| ATOM | 108 | CB | ASN | A | 15 | −19.848 | 20.545 | −37.073 | 1.00 | 16.07 |
| ATOM | 109 | CG | ASN | A | 15 | −19.232 | 21.010 | −38.388 | 1.00 | 18.31 |
| ATOM | 110 | OD1 | ASN | A | 15 | −19.565 | 20.487 | −39.431 | 1.00 | 17.60 |
| ATOM | 111 | ND2 | ASN | A | 15 | −18.312 | 21.987 | −38.325 | 1.00 | 21.40 |
| ATOM | 112 | C | ASN | A | 15 | −17.736 | 19.450 | −36.405 | 1.00 | 15.35 |
| ATOM | 113 | O | ASN | A | 15 | −16.926 | 18.954 | −37.198 | 1.00 | 15.29 |
| ATOM | 114 | N | ASN | A | 16 | −17.385 | 20.180 | −35.355 | 1.00 | 14.82 |
| ATOM | 115 | CA | ASN | A | 16 | −15.992 | 20.555 | −35.144 | 1.00 | 15.27 |
| ATOM | 116 | CB | ASN | A | 16 | −15.872 | 21.693 | −34.148 | 1.00 | 15.41 |
| ATOM | 117 | CG | ASN | A | 16 | −16.276 | 23.023 | −34.737 | 1.00 | 16.53 |
| ATOM | 118 | OD1 | ASN | A | 16 | −16.517 | 23.136 | −35.954 | 1.00 | 18.08 |
| ATOM | 119 | ND2 | ASN | A | 16 | −16.326 | 24.050 | −33.896 | 1.00 | 16.35 |
| ATOM | 120 | C | ASN | A | 16 | −15.159 | 19.362 | −34.723 | 1.00 | 14.91 |
| ATOM | 121 | O | ASN | A | 16 | −13.975 | 19.261 | −35.099 | 1.00 | 15.34 |
| ATOM | 122 | N | LEU | A | 17 | −15.771 | 18.460 | −33.956 | 1.00 | 14.29 |
| ATOM | 123 | CA | LEU | A | 17 | −15.114 | 17.191 | −33.610 | 1.00 | 13.90 |
| ATOM | 124 | CB | LEU | A | 17 | −16.003 | 16.346 | −32.672 | 1.00 | 13.94 |
| ATOM | 125 | CG | LEU | A | 17 | −15.351 | 15.065 | −32.133 | 1.00 | 16.81 |
| ATOM | 126 | CD1 | LEU | A | 17 | −15.933 | 14.708 | −30.779 | 1.00 | 20.06 |
| ATOM | 127 | CD2 | LEU | A | 17 | −15.484 | 13.880 | −33.097 | 1.00 | 19.31 |
| ATOM | 128 | C | LEU | A | 17 | −14.763 | 16.409 | −34.880 | 1.00 | 14.06 |
| ATOM | 129 | O | LEU | A | 17 | −13.613 | 15.957 | −35.073 | 1.00 | 12.69 |
| ATOM | 130 | N | LEU | A | 18 | −15.774 | 16.215 | −35.730 | 1.00 | 13.19 |
| ATOM | 131 | CA | LEU | A | 18 | −15.589 | 15.441 | −36.957 | 1.00 | 14.25 |
| ATOM | 132 | CB | LEU | A | 18 | −16.952 | 15.027 | −37.545 | 1.00 | 13.56 |
| ATOM | 133 | CG | LEU | A | 18 | −17.717 | 14.013 | −36.684 | 1.00 | 16.49 |
| ATOM | 134 | CD1 | LEU | A | 18 | −19.171 | 13.874 | −37.165 | 1.00 | 16.33 |
| ATOM | 135 | CD2 | LEU | A | 18 | −17.020 | 12.647 | −36.655 | 1.00 | 18.51 |
| ATOM | 136 | C | LEU | A | 18 | −14.703 | 16.132 | −38.007 | 1.00 | 13.49 |
| ATOM | 137 | O | LEU | A | 18 | −14.083 | 15.435 | −38.820 | 1.00 | 14.69 |
| ATOM | 138 | N | CYS | A | 19 | −14.613 | 17.462 | −37.964 | 1.00 | 13.01 |
| ATOM | 139 | CA | CYS | A | 19 | −13.629 | 18.223 | −38.760 | 1.00 | 13.22 |
| ATOM | 140 | CB | CYS | A | 19 | −13.796 | 19.738 | −38.556 | 1.00 | 14.20 |
| ATOM | 141 | SG | CYS | A | 19 | −15.125 | 20.407 | −39.642 | 1.00 | 16.22 |
| ATOM | 142 | C | CYS | A | 19 | −12.182 | 17.808 | −38.450 | 1.00 | 13.86 |
| ATOM | 143 | O | CYS | A | 19 | −11.278 | 18.043 | −39.259 | 1.00 | 13.42 |
| ATOM | 144 | N | ASN | A | 20 | −11.968 | 17.219 | −37.272 | 1.00 | 13.21 |
| ATOM | 145 | CA | ASN | A | 20 | −10.594 | 16.850 | −36.830 | 1.00 | 13.62 |
| ATOM | 146 | CB | ASN | A | 20 | −10.394 | 17.184 | −35.324 | 1.00 | 13.52 |
| ATOM | 147 | CG | ASN | A | 20 | −10.242 | 18.687 | −35.055 | 1.00 | 16.17 |
| ATOM | 148 | OD1 | ASN | A | 20 | −10.035 | 19.119 | −33.897 | 1.00 | 17.34 |
| ATOM | 149 | ND2 | ASN | A | 20 | −10.343 | 19.486 | −36.090 | 1.00 | 11.87 |
| ATOM | 150 | C | ASN | A | 20 | −10.262 | 15.381 | −37.116 | 1.00 | 13.99 |
| ATOM | 151 | O | ASN | A | 20 | −9.238 | 14.857 | −36.646 | 1.00 | 14.28 |
| ATOM | 152 | N | VAL | A | 21 | −11.123 | 14.705 | −37.875 | 1.00 | 13.41 |
| ATOM | 153 | CA | VAL | A | 21 | −10.923 | 13.287 | −38.167 | 1.00 | 14.20 |
| ATOM | 154 | CB | VAL | A | 21 | −12.177 | 12.448 | −37.827 | 1.00 | 14.30 |
| ATOM | 155 | CG1 | VAL | A | 21 | −11.953 | 10.971 | −38.189 | 1.00 | 15.30 |
| ATOM | 156 | CG2 | VAL | A | 21 | −12.517 | 12.553 | −36.312 | 1.00 | 14.17 |
| ATOM | 157 | C | VAL | A | 21 | −10.551 | 13.136 | −39.644 | 1.00 | 14.35 |
| ATOM | 158 | O | VAL | A | 21 | −11.255 | 13.642 | −40.491 | 1.00 | 15.68 |
| ATOM | 159 | N | GLY | A | 22 | −9.461 | 12.449 | −39.953 | 1.00 | 15.67 |
| ATOM | 160 | CA | GLY | A | 22 | −9.061 | 12.300 | −41.377 | 1.00 | 15.70 |
| ATOM | 161 | C | GLY | A | 22 | −9.843 | 11.182 | −42.060 | 1.00 | 17.34 |
| ATOM | 162 | O | GLY | A | 22 | −10.453 | 10.358 | −41.397 | 1.00 | 17.15 |
| ATOM | 163 | N | PRO | A | 23 | −9.806 | 11.117 | −43.404 | 1.00 | 18.42 |
| ATOM | 164 | CA | PRO | A | 23 | −9.009 | 11.946 | −44.278 | 1.00 | 18.20 |
| ATOM | 165 | CB | PRO | A | 23 | −8.716 | 10.990 | −45.446 | 1.00 | 18.64 |
| ATOM | 166 | CG | PRO | A | 23 | −9.983 | 10.171 | −45.560 | 1.00 | 18.81 |
| ATOM | 167 | CD | PRO | A | 23 | −10.568 | 10.092 | −44.153 | 1.00 | 18.59 |
| ATOM | 168 | C | PRO | A | 23 | −9.761 | 13.182 | −44.753 | 1.00 | 19.05 |
| ATOM | 169 | O | PRO | A | 23 | −9.183 | 14.055 | −45.426 | 1.00 | 19.36 |
| ATOM | 170 | N | ASP | A | 24 | −11.034 | 13.288 | −44.385 | 1.00 | 18.76 |
| ATOM | 171 | CA | ASP | A | 24 | −11.878 | 14.305 | −44.996 | 1.00 | 19.39 |
| ATOM | 172 | CB | ASP | A | 24 | −13.015 | 13.636 | −45.781 | 1.00 | 20.87 |
| ATOM | 173 | CG | ASP | A | 24 | −13.920 | 12.784 | −44.913 | 1.00 | 24.34 |
| ATOM | 174 | OD1 | ASP | A | 24 | −13.502 | 12.291 | −43.839 | 1.00 | 27.70 |
| ATOM | 175 | OD2 | ASP | A | 24 | −15.079 | 12.580 | −45.330 | 1.00 | 28.83 |
| ATOM | 176 | C | ASP | A | 24 | −12.452 | 15.372 | −44.061 | 1.00 | 18.08 |
| ATOM | 177 | O | ASP | A | 24 | −13.208 | 16.245 | −44.509 | 1.00 | 17.78 |
| ATOM | 178 | N | GLY | A | 25 | −12.100 | 15.331 | −42.775 | 1.00 | 16.65 |
| ATOM | 179 | CA | GLY | A | 25 | −12.634 | 16.343 | −41.852 | 1.00 | 16.02 |
| ATOM | 180 | C | GLY | A | 25 | −12.152 | 17.718 | −42.292 | 1.00 | 15.70 |
| ATOM | 181 | O | GLY | A | 25 | −11.033 | 17.849 | −42.811 | 1.00 | 16.22 |
| ATOM | 182 | N | CYS | A | 26 | −12.979 | 18.752 | −42.086 | 1.00 | 15.10 |
| ATOM | 183 | CA | CYS | A | 26 | −12.698 | 20.078 | −42.637 | 1.00 | 15.46 |
| ATOM | 184 | CB | CYS | A | 26 | −13.899 | 21.037 | −42.475 | 1.00 | 15.47 |
| ATOM | 185 | SG | CYS | A | 26 | −14.147 | 21.739 | −40.786 | 1.00 | 16.91 |
| ATOM | 186 | C | CYS | A | 26 | −11.407 | 20.731 | −42.116 | 1.00 | 15.65 |
| ATOM | 187 | O | CYS | A | 26 | −10.896 | 21.643 | −42.763 | 1.00 | 15.80 |
| ATOM | 188 | N | ARG | A | 27 | −10.879 | 20.259 | −40.973 | 1.00 | 15.02 |
| ATOM | 189 | CA | ARG | A | 27 | −9.615 | 20.808 | −40.443 | 1.00 | 14.56 |
| ATOM | 190 | CB | ARG | A | 27 | −9.819 | 21.480 | −39.066 | 1.00 | 15.00 |
| ATOM | 191 | CG | ARG | A | 27 | −10.695 | 22.728 | −39.164 | 1.00 | 15.15 |
| ATOM | 192 | CD | ARG | A | 27 | −10.826 | 23.551 | −37.888 | 1.00 | 14.30 |
| ATOM | 193 | NE | ARG | A | 27 | −11.874 | 24.566 | −38.080 | 1.00 | 15.05 |
| ATOM | 194 | CZ | ARG | A | 27 | −13.160 | 24.420 | −37.761 | 1.00 | 17.96 |
| ATOM | 195 | NH1 | ARG | A | 27 | −13.623 | 23.293 | −37.211 | 1.00 | 17.37 |
| ATOM | 196 | NH2 | ARG | A | 27 | −14.009 | 25.415 | −38.025 | 1.00 | 19.55 |
| ATOM | 197 | C | ARG | A | 27 | −8.489 | 19.776 | −40.394 | 1.00 | 15.49 |
| ATOM | 198 | O | ARG | A | 27 | −7.389 | 20.079 | −39.888 | 1.00 | 15.40 |
| ATOM | 199 | N | ALA | A | 28 | −8.768 | 18.577 | −40.910 | 1.00 | 15.17 |
| ATOM | 200 | CA | ALA | A | 28 | −7.805 | 17.484 | −40.988 | 1.00 | 16.06 |
| ATOM | 201 | CB | ALA | A | 28 | −8.163 | 16.374 | −39.975 | 1.00 | 15.52 |
| ATOM | 202 | C | ALA | A | 28 | −7.744 | 16.913 | −42.394 | 1.00 | 16.96 |
| ATOM | 203 | O | ALA | A | 28 | −7.453 | 15.730 | −42.581 | 1.00 | 17.60 |
| ATOM | 204 | N | PHE | A | 29 | −8.028 | 17.756 | −43.380 | 1.00 | 17.45 |
| ATOM | 205 | CA | PHE | A | 29 | −8.188 | 17.272 | −44.744 | 1.00 | 18.68 |
| ATOM | 206 | CB | PHE | A | 29 | −8.728 | 18.376 | −45.636 | 1.00 | 19.45 |
| ATOM | 207 | CG | PHE | A | 29 | −9.299 | 17.864 | −46.919 | 1.00 | 20.86 |
| ATOM | 208 | CD1 | PHE | A | 29 | −8.515 | 17.827 | −48.071 | 1.00 | 23.76 |
| ATOM | 209 | CE1 | PHE | A | 29 | −9.042 | 17.343 | −49.267 | 1.00 | 25.46 |
| ATOM | 210 | CZ | PHE | A | 29 | −10.357 | 16.889 | −49.318 | 1.00 | 22.85 |
| ATOM | 211 | CE2 | PHE | A | 29 | −11.151 | 16.924 | −48.180 | 1.00 | 24.78 |
| ATOM | 212 | CD2 | PHE | A | 29 | −10.611 | 17.408 | −46.973 | 1.00 | 22.71 |
| ATOM | 213 | C | PHE | A | 29 | −6.853 | 16.783 | −45.296 | 1.00 | 19.10 |
| ATOM | 214 | O | PHE | A | 29 | −5.862 | 17.501 | −45.224 | 1.00 | 19.40 |
| ATOM | 215 | N | GLY | A | 30 | −6.830 | 15.558 | −45.816 | 1.00 | 18.73 |
| ATOM | 216 | CA | GLY | A | 30 | −5.603 | 15.008 | −46.398 | 1.00 | 19.00 |
| ATOM | 217 | C | GLY | A | 30 | −4.717 | 14.307 | −45.399 | 1.00 | 19.69 |
| ATOM | 218 | O | GLY | A | 30 | −3.657 | 13.809 | −45.768 | 1.00 | 19.61 |
| ATOM | 219 | N | THR | A | 31 | −5.133 | 14.255 | −44.123 | 1.00 | 18.86 |
| ATOM | 220 | CA | THR | A | 31 | −4.450 | 13.384 | −43.165 | 1.00 | 19.14 |
| ATOM | 221 | CB | THR | A | 31 | −4.846 | 13.689 | −41.689 | 1.00 | 18.79 |
| ATOM | 222 | OG1 | THR | A | 31 | −6.265 | 13.579 | −41.559 | 1.00 | 18.61 |
| ATOM | 223 | CG2 | THR | A | 31 | −4.410 | 15.106 | −41.262 | 1.00 | 16.47 |
| ATOM | 224 | C | THR | A | 31 | −4.812 | 11.925 | −43.498 | 1.00 | 19.11 |
| ATOM | 225 | O | THR | A | 31 | −5.713 | 11.661 | −44.313 | 1.00 | 19.69 |
| ATOM | 226 | N | SER | A | 32 | −4.107 | 10.982 | −42.881 | 1.00 | 19.74 |
| ATOM | 227 | CA | SER | A | 32 | −4.367 | 9.554 | −43.094 | 1.00 | 20.00 |
| ATOM | 228 | CB | SER | A | 32 | −3.411 | 8.722 | −42.248 | 1.00 | 20.73 |
| ATOM | 229 | OG | SER | A | 32 | −2.064 | 8.973 | −42.612 | 1.00 | 21.56 |
| ATOM | 230 | C | SER | A | 32 | −5.806 | 9.217 | −42.704 | 1.00 | 20.57 |
| ATOM | 231 | O | SER | A | 32 | −6.334 | 9.778 | −41.732 | 1.00 | 20.70 |
| ATOM | 232 | N | ALA | A | 33 | −6.443 | 8.319 | −43.452 | 1.00 | 19.94 |
| ATOM | 233 | CA | ALA | A | 33 | −7.768 | 7.823 | −43.068 | 1.00 | 19.61 |
| ATOM | 234 | CB | ALA | A | 33 | −8.232 | 6.729 | −44.035 | 1.00 | 19.31 |
| ATOM | 235 | C | ALA | A | 33 | −7.764 | 7.285 | −41.637 | 1.00 | 19.10 |
| ATOM | 236 | O | ALA | A | 33 | −6.906 | 6.482 | −41.264 | 1.00 | 19.49 |
| ATOM | 237 | N | GLY | A | 34 | −8.742 | 7.719 | −40.856 | 1.00 | 17.74 |
| ATOM | 238 | CA | GLY | A | 34 | −8.878 | 7.282 | −39.473 | 1.00 | 18.31 |
| ATOM | 239 | C | GLY | A | 34 | −7.988 | 8.020 | −38.473 | 1.00 | 18.48 |
| ATOM | 240 | O | GLY | A | 34 | −8.050 | 7.739 | −37.271 | 1.00 | 19.07 |
| ATOM | 241 | N | ALA | A | 35 | −7.173 | 8.959 | −38.937 | 1.00 | 17.05 |
| ATOM | 242 | CA | ALA | A | 35 | −6.329 | 9.723 | −38.000 | 1.00 | 17.17 |
| ATOM | 243 | CB | ALA | A | 35 | −5.167 | 10.376 | −38.730 | 1.00 | 17.10 |
| ATOM | 244 | C | ALA | A | 35 | −7.173 | 10.784 | −37.271 | 1.00 | 16.55 |
| ATOM | 245 | O | ALA | A | 35 | −8.174 | 11.271 | −37.808 | 1.00 | 17.35 |
| ATOM | 246 | N | VAL | A | 36 | −6.793 | 11.130 | −36.051 | 1.00 | 15.39 |
| ATOM | 247 | CA | VAL | A | 36 | −7.490 | 12.198 | −35.328 | 1.00 | 14.41 |
| ATOM | 248 | CB | VAL | A | 36 | −8.142 | 11.687 | −34.031 | 1.00 | 15.02 |
| ATOM | 249 | CG1 | VAL | A | 36 | −8.903 | 12.828 | −33.349 | 1.00 | 16.72 |
| ATOM | 250 | CG2 | VAL | A | 36 | −9.081 | 10.520 | −34.336 | 1.00 | 16.45 |
| ATOM | 251 | C | VAL | A | 36 | −6.407 | 13.201 | −34.944 | 1.00 | 14.36 |
| ATOM | 252 | O | VAL | A | 36 | −5.421 | 12.816 | −34.311 | 1.00 | 14.44 |
| ATOM | 253 | N | ILE | A | 37 | −6.566 | 14.454 | −35.331 | 1.00 | 13.68 |
| ATOM | 254 | CA | ILE | A | 37 | −5.614 | 15.470 | −34.893 | 1.00 | 13.67 |
| ATOM | 255 | CB | ILE | A | 37 | −5.528 | 16.687 | −35.849 | 1.00 | 13.66 |
| ATOM | 256 | CG1 | ILE | A | 37 | −6.847 | 17.486 | −35.901 | 1.00 | 14.31 |
| ATOM | 257 | CD1 | ILE | A | 37 | −6.773 | 18.712 | −36.864 | 1.00 | 14.21 |
| ATOM | 258 | CG2 | ILE | A | 37 | −5.158 | 16.214 | −37.260 | 1.00 | 14.62 |
| ATOM | 259 | C | ILE | A | 37 | −6.041 | 15.908 | −33.505 | 1.00 | 13.27 |
| ATOM | 260 | O | ILE | A | 37 | −7.235 | 16.011 | −33.224 | 1.00 | 12.99 |
| ATOM | 261 | N | ALA | A | 38 | −5.081 | 16.159 | −32.630 | 1.00 | 13.03 |
| ATOM | 262 | CA | ALA | A | 38 | −5.445 | 16.697 | −31.333 | 1.00 | 12.81 |
| ATOM | 263 | CB | ALA | A | 38 | −4.235 | 16.680 | −30.377 | 1.00 | 12.73 |
| ATOM | 264 | C | ALA | A | 38 | −6.046 | 18.122 | −31.497 | 1.00 | 12.45 |
| ATOM | 265 | O | ALA | A | 38 | −6.939 | 18.503 | −30.775 | 1.00 | 12.23 |
| ATOM | 266 | N | SER | A | 39 | −5.555 | 18.870 | −32.482 | 1.00 | 12.90 |
| ATOM | 267 | CA | SER | A | 39 | −5.973 | 20.246 | −32.769 | 1.00 | 12.85 |
| ATOM | 268 | CB | SER | A | 39 | −5.512 | 21.211 | −31.657 | 1.00 | 12.63 |
| ATOM | 269 | OG | SER | A | 39 | −5.312 | 22.563 | −32.108 | 1.00 | 12.57 |
| ATOM | 270 | C | SER | A | 39 | −5.281 | 20.593 | −34.090 | 1.00 | 13.33 |
| ATOM | 271 | O | SER | A | 39 | −4.215 | 20.043 | −34.376 | 1.00 | 13.48 |
| ATOM | 272 | N | PRO | A | 40 | −5.880 | 21.500 | −34.885 | 1.00 | 13.12 |
| ATOM | 273 | CA | PRO | A | 40 | −5.248 | 21.999 | −36.108 | 1.00 | 13.76 |
| ATOM | 274 | CB | PRO | A | 40 | −6.407 | 22.689 | −36.860 | 1.00 | 14.41 |
| ATOM | 275 | CG | PRO | A | 40 | −7.386 | 23.045 | −35.797 | 1.00 | 14.32 |
| ATOM | 276 | CD | PRO | A | 40 | −7.223 | 22.081 | −34.665 | 1.00 | 13.18 |
| ATOM | 277 | C | PRO | A | 40 | −4.126 | 23.010 | −35.860 | 1.00 | 14.27 |
| ATOM | 278 | O | PRO | A | 40 | −3.474 | 23.420 | −36.824 | 1.00 | 14.43 |
| ATOM | 279 | N | SER | A | 41 | −3.864 | 23.381 | −34.599 | 1.00 | 13.42 |
| ATOM | 280 | CA | SER | A | 41 | −2.799 | 24.336 | −34.318 | 1.00 | 14.56 |
| ATOM | 281 | CB | SER | A | 41 | −2.788 | 24.817 | −32.840 | 1.00 | 14.40 |
| ATOM | 282 | OG | SER | A | 41 | −3.962 | 25.574 | −32.534 | 1.00 | 16.91 |
| ATOM | 283 | C | SER | A | 41 | −1.446 | 23.713 | −34.676 | 1.00 | 14.51 |
| ATOM | 284 | O | SER | A | 41 | −1.123 | 22.626 | −34.218 | 1.00 | 13.96 |
| ATOM | 285 | N | THR | A | 42 | −0.650 | 24.433 | −35.470 | 1.00 | 15.63 |
| ATOM | 286 | CA | THR | A | 42 | 0.652 | 23.924 | −35.919 | 1.00 | 16.17 |
| ATOM | 287 | CB | THR | A | 42 | 0.750 | 23.997 | −37.458 | 1.00 | 17.02 |
| ATOM | 288 | OG1 | THR | A | 42 | 0.267 | 25.283 | −37.890 | 1.00 | 17.03 |
| ATOM | 289 | CG2 | THR | A | 42 | −0.110 | 22.906 | −38.078 | 1.00 | 16.03 |
| ATOM | 290 | C | THR | A | 42 | 1.814 | 24.732 | −35.322 | 1.00 | 17.44 |
| ATOM | 291 | O | THR | A | 42 | 2.967 | 24.297 | −35.382 | 1.00 | 17.10 |
| ATOM | 292 | N | ILE | A | 43 | 1.509 | 25.913 | −34.787 | 1.00 | 18.37 |
| ATOM | 293 | CA | ILE | A | 43 | 2.510 | 26.786 | −34.171 | 1.00 | 20.62 |
| ATOM | 294 | CB | ILE | A | 43 | 2.923 | 27.952 | −35.114 | 1.00 | 20.73 |
| ATOM | 295 | CG1 | ILE | A | 43 | 3.550 | 27.428 | −36.411 | 1.00 | 21.88 |
| ATOM | 296 | CD1 | ILE | A | 43 | 3.788 | 28.507 | −37.508 | 1.00 | 22.99 |
| ATOM | 297 | CG2 | ILE | A | 43 | 3.895 | 28.910 | −34.409 | 1.00 | 21.41 |
| ATOM | 298 | C | ILE | A | 43 | 1.908 | 27.395 | −32.935 | 1.00 | 21.00 |
| ATOM | 299 | O | ILE | A | 43 | 0.796 | 27.921 | −32.995 | 1.00 | 21.76 |
| ATOM | 300 | N | ASP | A | 44 | 2.683 | 27.470 | −31.874 | 1.00 | 21.61 |
| ATOM | 301 | CA | ASP | A | 44 | 2.237 | 27.975 | −30.572 | 1.00 | 23.04 |
| ATOM | 302 | CB | ASP | A | 44 | 2.408 | 29.506 | −30.492 | 1.00 | 24.75 |
| ATOM | 303 | CG | ASP | A | 44 | 2.170 | 30.064 | −29.098 | 1.00 | 31.28 |
| ATOM | 304 | OD1 | ASP | A | 44 | 2.362 | 29.340 | −28.094 | 1.00 | 37.92 |
| ATOM | 305 | OD2 | ASP | A | 44 | 1.766 | 31.260 | −28.997 | 1.00 | 40.00 |
| ATOM | 306 | C | ASP | A | 44 | 0.782 | 27.608 | −30.196 | 1.00 | 21.65 |
| ATOM | 307 | O | ASP | A | 44 | −0.046 | 28.502 | −29.981 | 1.00 | 22.69 |
| ATOM | 308 | N | PRO | A | 45 | 0.441 | 26.449 | −29.805 | 1.00 | 19.86 |
| ATOM | 309 | CA | PRO | A | 45 | 1.356 | 25.320 | −29.775 | 1.00 | 18.66 |
| ATOM | 310 | CB | PRO | A | 45 | 0.883 | 24.549 | −28.549 | 1.00 | 18.40 |
| ATOM | 311 | CG | PRO | A | 45 | −0.653 | 24.763 | −28.586 | 1.00 | 18.13 |
| ATOM | 312 | CD | PRO | A | 45 | −0.899 | 26.066 | −29.318 | 1.00 | 20.04 |
| ATOM | 313 | C | PRO | A | 45 | 1.253 | 24.454 | −31.026 | 1.00 | 17.74 |
| ATOM | 314 | O | PRO | A | 45 | 0.368 | 24.652 | −31.858 | 1.00 | 17.36 |
| ATOM | 315 | N | ASP | A | 46 | 2.178 | 23.512 | −31.160 | 1.00 | 15.95 |
| ATOM | 316 | CA | ASP | A | 46 | 2.124 | 22.573 | −32.275 | 1.00 | 14.75 |
| ATOM | 317 | CB | ASP | A | 46 | 3.551 | 22.255 | −32.738 | 1.00 | 14.59 |
| ATOM | 318 | CG | ASP | A | 46 | 3.601 | 21.161 | −33.818 | 1.00 | 16.17 |
| ATOM | 319 | OD1 | ASP | A | 46 | 2.543 | 20.787 | −34.389 | 1.00 | 15.61 |
| ATOM | 320 | OD2 | ASP | A | 46 | 4.712 | 20.641 | −34.054 | 1.00 | 20.18 |
| ATOM | 321 | C | ASP | A | 46 | 1.436 | 21.303 | −31.748 | 1.00 | 13.83 |
| ATOM | 322 | O | ASP | A | 46 | 2.081 | 20.489 | −31.089 | 1.00 | 13.59 |
| ATOM | 323 | N | TYR | A | 47 | 0.126 | 21.165 | −31.992 | 1.00 | 11.86 |
| ATOM | 324 | CA | TYR | A | 47 | −0.621 | 19.975 | −31.580 | 1.00 | 12.11 |
| ATOM | 325 | CB | TYR | A | 47 | −1.895 | 20.387 | −30.854 | 1.00 | 11.69 |
| ATOM | 326 | CG | TYR | A | 47 | −1.773 | 20.722 | −29.377 | 1.00 | 12.59 |
| ATOM | 327 | CD1 | TYR | A | 47 | −0.589 | 21.236 | −28.827 | 1.00 | 13.54 |
| ATOM | 328 | CE1 | TYR | A | 47 | −0.524 | 21.586 | −27.462 | 1.00 | 12.81 |
| ATOM | 329 | CZ | TYR | A | 47 | −1.652 | 21.407 | −26.673 | 1.00 | 13.40 |
| ATOM | 330 | OH | TYR | A | 47 | −1.620 | 21.755 | −25.354 | 1.00 | 13.08 |
| ATOM | 331 | CE2 | TYR | A | 47 | −2.825 | 20.887 | −27.208 | 1.00 | 12.02 |
| ATOM | 332 | CD2 | TYR | A | 47 | −2.876 | 20.540 | −28.532 | 1.00 | 12.76 |
| ATOM | 333 | C | TYR | A | 47 | −0.994 | 19.090 | −32.772 | 1.00 | 11.46 |
| ATOM | 334 | O | TYR | A | 47 | −1.885 | 18.239 | −32.692 | 1.00 | 11.66 |
| ATOM | 335 | N | TYR | A | 48 | −0.316 | 19.301 | −33.893 | 1.00 | 12.26 |
| ATOM | 336 | CA | TYR | A | 48 | −0.697 | 18.639 | −35.132 | 1.00 | 12.80 |
| ATOM | 337 | CB | TYR | A | 48 | −0.323 | 19.509 | −36.348 | 1.00 | 12.75 |
| ATOM | 338 | CG | TYR | A | 48 | −1.134 | 19.146 | −37.569 | 1.00 | 13.21 |
| ATOM | 339 | CD1 | TYR | A | 48 | −2.492 | 19.482 | −37.652 | 1.00 | 14.46 |
| ATOM | 340 | CE1 | TYR | A | 48 | −3.254 | 19.124 | −38.767 | 1.00 | 15.86 |
| ATOM | 341 | CZ | TYR | A | 48 | −2.643 | 18.453 | −39.823 | 1.00 | 14.62 |
| ATOM | 342 | OH | TYR | A | 48 | −3.390 | 18.106 | −40.936 | 1.00 | 16.20 |
| ATOM | 343 | CE2 | TYR | A | 48 | −1.295 | 18.086 | −39.756 | 1.00 | 15.86 |
| ATOM | 344 | CD2 | TYR | A | 48 | −0.543 | 18.456 | −38.638 | 1.00 | 13.44 |
| ATOM | 345 | C | TYR | A | 48 | −0.072 | 17.245 | −35.187 | 1.00 | 13.47 |
| ATOM | 346 | O | TYR | A | 48 | 0.877 | 16.986 | −35.940 | 1.00 | 13.95 |
| ATOM | 347 | N | TYR | A | 49 | −0.592 | 16.360 | −34.338 | 1.00 | 13.13 |
| ATOM | 348 | CA | TYR | A | 49 | −0.131 | 14.987 | −34.171 | 1.00 | 13.51 |
| ATOM | 349 | CB | TYR | A | 49 | 0.887 | 14.842 | −33.009 | 1.00 | 13.11 |
| ATOM | 350 | CG | TYR | A | 49 | 2.133 | 15.662 | −33.216 | 1.00 | 13.90 |
| ATOM | 351 | CD1 | TYR | A | 49 | 3.193 | 15.174 | −33.996 | 1.00 | 13.54 |
| ATOM | 352 | CE1 | TYR | A | 49 | 4.354 | 15.964 | −34.216 | 1.00 | 13.41 |
| ATOM | 353 | CZ | TYR | A | 49 | 4.419 | 17.225 | −33.665 | 1.00 | 14.69 |
| ATOM | 354 | OH | TYR | A | 49 | 5.511 | 18.016 | −33.883 | 1.00 | 17.21 |
| ATOM | 355 | CE2 | TYR | A | 49 | 3.365 | 17.737 | −32.906 | 1.00 | 13.49 |
| ATOM | 356 | CD2 | TYR | A | 49 | 2.227 | 16.952 | −32.698 | 1.00 | 13.78 |
| ATOM | 357 | C | TYR | A | 49 | −1.349 | 14.181 | −33.783 | 1.00 | 13.93 |
| ATOM | 358 | O | TYR | A | 49 | −2.390 | 14.759 | −33.406 | 1.00 | 13.00 |
| ATOM | 359 | N | MET | A | 50 | −1.203 | 12.857 | −33.839 | 1.00 | 13.66 |
| ATOM | 360 | CA | MET | A | 50 | −2.241 | 11.940 | −33.365 | 1.00 | 14.56 |
| ATOM | 361 | CB | MET | A | 50 | −2.447 | 10.822 | −34.381 | 1.00 | 15.21 |
| ATOM | 362 | CG | MET | A | 50 | −3.532 | 9.811 | −33.947 | 1.00 | 15.64 |
| ATOM | 363 | SD | MET | A | 50 | −3.996 | 8.804 | −35.361 | 1.00 | 19.52 |
| ATOM | 364 | CE | MET | A | 50 | −5.204 | 7.742 | −34.566 | 1.00 | 17.12 |
| ATOM | 365 | C | MET | A | 50 | −1.797 | 11.323 | −32.060 | 1.00 | 14.38 |
| ATOM | 366 | O | MET | A | 50 | −0.806 | 10.583 | −32.024 | 1.00 | 13.80 |
| ATOM | 367 | N | TRP | A | 51 | −2.528 | 11.608 | −30.984 | 1.00 | 13.47 |
| ATOM | 368 | CA | TRP | A | 51 | −2.265 | 10.965 | −29.720 | 1.00 | 13.13 |
| ATOM | 369 | CB | TRP | A | 51 | −2.598 | 11.930 | −28.585 | 1.00 | 12.85 |
| ATOM | 370 | CG | TRP | A | 51 | −1.478 | 12.809 | −28.116 | 1.00 | 13.64 |
| ATOM | 371 | CD1 | TRP | A | 51 | −0.671 | 12.604 | −27.011 | 1.00 | 13.49 |
| ATOM | 372 | NE1 | TRP | A | 51 | 0.211 | 13.657 | −26.864 | 1.00 | 12.36 |
| ATOM | 373 | CE2 | TRP | A | 51 | −0.023 | 14.573 | −27.858 | 1.00 | 11.83 |
| ATOM | 374 | CD2 | TRP | A | 51 | −1.076 | 14.065 | −28.674 | 1.00 | 13.12 |
| ATOM | 375 | CE3 | TRP | A | 51 | −1.506 | 14.825 | −29.772 | 1.00 | 11.07 |
| ATOM | 376 | CZ3 | TRP | A | 51 | −0.859 | 16.061 | −30.035 | 1.00 | 12.87 |
| ATOM | 377 | CH2 | TRP | A | 51 | 0.193 | 16.522 | −29.218 | 1.00 | 13.24 |
| ATOM | 378 | CZ2 | TRP | A | 51 | 0.618 | 15.806 | −28.127 | 1.00 | 12.61 |
| ATOM | 379 | C | TRP | A | 51 | −3.136 | 9.732 | −29.575 | 1.00 | 13.35 |
| ATOM | 380 | O | TRP | A | 51 | −4.322 | 9.769 | −29.907 | 1.00 | 12.89 |
| ATOM | 381 | N | THR | A | 52 | −2.576 | 8.652 | −29.024 | 1.00 | 13.20 |
| ATOM | 382 | CA | THR | A | 52 | −3.386 | 7.462 | −28.753 | 1.00 | 13.02 |
| ATOM | 383 | CB | THR | A | 52 | −2.520 | 6.300 | −28.235 | 1.00 | 13.66 |
| ATOM | 384 | OG1 | THR | A | 52 | −1.553 | 5.999 | −29.246 | 1.00 | 15.07 |
| ATOM | 385 | CG2 | THR | A | 52 | −3.341 | 5.026 | −27.952 | 1.00 | 12.21 |
| ATOM | 386 | C | THR | A | 52 | −4.533 | 7.807 | −27.800 | 1.00 | 12.48 |
| ATOM | 387 | O | THR | A | 52 | −5.670 | 7.402 | −28.034 | 1.00 | 12.78 |
| ATOM | 388 | N | ARG | A | 53 | −4.224 | 8.556 | −26.747 | 1.00 | 12.03 |
| ATOM | 389 | CA | ARG | A | 53 | −5.238 | 8.868 | −25.737 | 1.00 | 11.89 |
| ATOM | 390 | CB | ARG | A | 53 | −4.607 | 9.570 | −24.545 | 1.00 | 11.46 |
| ATOM | 391 | CG | ARG | A | 53 | −5.611 | 10.330 | −23.618 | 1.00 | 13.19 |
| ATOM | 392 | CD | ARG | A | 53 | −4.896 | 10.881 | −22.375 | 1.00 | 11.14 |
| ATOM | 393 | NE | ARG | A | 53 | −3.793 | 11.694 | −22.819 | 1.00 | 12.52 |
| ATOM | 394 | CZ | ARG | A | 53 | −2.509 | 11.330 | −22.769 | 1.00 | 13.67 |
| ATOM | 395 | NH1 | ARG | A | 53 | −2.148 | 10.182 | −22.180 | 1.00 | 13.97 |
| ATOM | 396 | NH2 | ARG | A | 53 | −1.590 | 12.151 | −23.270 | 1.00 | 13.05 |
| ATOM | 397 | C | ARG | A | 53 | −6.395 | 9.709 | −26.319 | 1.00 | 12.45 |
| ATOM | 398 | O | ARG | A | 53 | −7.558 | 9.289 | −26.244 | 1.00 | 11.74 |
| ATOM | 399 | N | ASP | A | 54 | −6.090 | 10.885 | −26.874 | 1.00 | 11.73 |
| ATOM | 400 | CA | ASP | A | 54 | −7.169 | 11.747 | −27.385 | 1.00 | 11.90 |
| ATOM | 401 | CB | ASP | A | 54 | −6.638 | 13.018 | −28.053 | 1.00 | 12.25 |
| ATOM | 402 | CG | ASP | A | 54 | −5.794 | 13.879 | −27.120 | 1.00 | 13.97 |
| ATOM | 403 | OD1 | ASP | A | 54 | −4.983 | 13.332 | −26.354 | 1.00 | 13.57 |
| ATOM | 404 | OD2 | ASP | A | 54 | −5.910 | 15.110 | −27.215 | 1.00 | 13.88 |
| ATOM | 405 | C | ASP | A | 54 | −8.002 | 11.005 | −28.420 | 1.00 | 12.00 |
| ATOM | 406 | O | ASP | A | 54 | −9.236 | 11.138 | −28.454 | 1.00 | 10.97 |
| ATOM | 407 | N | SER | A | 55 | −7.334 | 10.250 | −29.297 | 1.00 | 11.19 |
| ATOM | 408 | CA | SER | A | 55 | −8.034 | 9.544 | −30.388 | 1.00 | 12.36 |
| ATOM | 409 | CB | SER | A | 55 | −7.017 | 8.901 | −31.340 | 1.00 | 13.05 |
| ATOM | 410 | OG | SER | A | 55 | −6.171 | 9.930 | −31.882 | 1.00 | 14.23 |
| ATOM | 411 | C | SER | A | 55 | −8.996 | 8.489 | −29.838 | 1.00 | 12.57 |
| ATOM | 412 | O | SER | A | 55 | −10.130 | 8.348 | −30.327 | 1.00 | 12.76 |
| ATOM | 413 | N | ALA | A | 56 | −8.556 | 7.764 | −28.819 | 1.00 | 12.60 |
| ATOM | 414 | CA | ALA | A | 56 | −9.373 | 6.718 | −28.218 | 1.00 | 13.25 |
| ATOM | 415 | CB | ALA | A | 56 | −8.517 | 5.830 | −27.329 | 1.00 | 12.73 |
| ATOM | 416 | C | ALA | A | 56 | −10.551 | 7.301 | −27.415 | 1.00 | 13.85 |
| ATOM | 417 | O | ALA | A | 56 | −11.640 | 6.723 | −27.409 | 1.00 | 14.51 |
| ATOM | 418 | N | LEU | A | 57 | −10.328 | 8.420 | −26.723 | 1.00 | 14.23 |
| ATOM | 419 | CA | LEU | A | 57 | −11.417 | 9.059 | −25.954 | 1.00 | 13.95 |
| ATOM | 420 | CB | LEU | A | 57 | −10.891 | 10.186 | −25.060 | 1.00 | 14.45 |
| ATOM | 421 | CG | LEU | A | 57 | −10.088 | 9.751 | −23.834 | 1.00 | 14.89 |
| ATOM | 422 | CD1 | LEU | A | 57 | −9.507 | 11.013 | −23.161 | 1.00 | 16.19 |
| ATOM | 423 | CD2 | LEU | A | 57 | −10.919 | 8.911 | −22.867 | 1.00 | 16.02 |
| ATOM | 424 | C | LEU | A | 57 | −12.483 | 9.609 | −26.886 | 1.00 | 13.92 |
| ATOM | 425 | O | LEU | A | 57 | −13.685 | 9.488 | −26.627 | 1.00 | 13.70 |
| ATOM | 426 | N | VAL | A | 58 | −12.027 | 10.199 | −27.975 | 1.00 | 13.15 |
| ATOM | 427 | CA | VAL | A | 58 | −12.920 | 10.751 | −28.989 | 1.00 | 15.20 |
| ATOM | 428 | CB | VAL | A | 58 | −12.133 | 11.605 | −30.031 | 1.00 | 14.52 |
| ATOM | 429 | CG1 | VAL | A | 58 | −12.970 | 11.861 | −31.302 | 1.00 | 17.86 |
| ATOM | 430 | CG2 | VAL | A | 58 | −11.704 | 12.954 | −29.393 | 1.00 | 15.79 |
| ATOM | 431 | C | VAL | A | 58 | −13.704 | 9.624 | −29.655 | 1.00 | 15.20 |
| ATOM | 432 | O | VAL | A | 58 | −14.930 | 9.718 | −29.784 | 1.00 | 15.30 |
| ATOM | 433 | N | PHE | A | 59 | −13.026 | 8.553 | −30.058 | 1.00 | 15.02 |
| ATOM | 434 | CA | PHE | A | 59 | −13.766 | 7.477 | −30.713 | 1.00 | 15.23 |
| ATOM | 435 | CB | PHE | A | 59 | −12.882 | 6.601 | −31.582 | 1.00 | 15.78 |
| ATOM | 436 | CG | PHE | A | 59 | −12.859 | 7.058 | −33.003 | 1.00 | 15.10 |
| ATOM | 437 | CD1 | PHE | A | 59 | −11.872 | 7.927 | −33.444 | 1.00 | 16.45 |
| ATOM | 438 | CE1 | PHE | A | 59 | −11.861 | 8.401 | −34.768 | 1.00 | 19.37 |
| ATOM | 439 | CZ | PHE | A | 59 | −12.876 | 8.026 | −35.644 | 1.00 | 16.44 |
| ATOM | 440 | CE2 | PHE | A | 59 | −13.901 | 7.165 | −35.186 | 1.00 | 16.90 |
| ATOM | 441 | CD2 | PHE | A | 59 | −13.895 | 6.709 | −33.882 | 1.00 | 16.17 |
| ATOM | 442 | C | PHE | A | 59 | −14.674 | 6.681 | −29.785 | 1.00 | 15.69 |
| ATOM | 443 | O | PHE | A | 59 | −15.699 | 6.175 | −30.220 | 1.00 | 15.46 |
| ATOM | 444 | N | LYS | A | 60 | −14.321 | 6.586 | −28.510 | 1.00 | 15.65 |
| ATOM | 445 | CA | LYS | A | 60 | −15.257 | 5.994 | −27.552 | 1.00 | 16.61 |
| ATOM | 446 | CB | LYS | A | 60 | −14.661 | 5.954 | −26.144 | 1.00 | 16.44 |
| ATOM | 447 | CG | LYS | A | 60 | −15.626 | 5.363 | −25.059 | 1.00 | 17.65 |
| ATOM | 448 | CD | LYS | A | 60 | −16.174 | 3.992 | −25.433 | 1.00 | 18.35 |
| ATOM | 449 | CE | LYS | A | 60 | −16.738 | 3.234 | −24.199 | 1.00 | 19.79 |
| ATOM | 450 | NZ | LYS | A | 60 | −17.819 | 3.976 | −23.512 | 1.00 | 18.40 |
| ATOM | 451 | C | LYS | A | 60 | −16.577 | 6.779 | −27.579 | 1.00 | 16.72 |
| ATOM | 452 | O | LYS | A | 60 | −17.663 | 6.182 | −27.681 | 1.00 | 17.08 |
| ATOM | 453 | N | ASN | A | 61 | −16.487 | 8.101 | −27.508 | 1.00 | 16.77 |
| ATOM | 454 | CA | ASN | A | 61 | −17.680 | 8.948 | −27.628 | 1.00 | 18.06 |
| ATOM | 455 | CB | ASN | A | 61 | −17.278 | 10.424 | −27.573 | 1.00 | 19.41 |
| ATOM | 456 | CG | ASN | A | 61 | −18.465 | 11.375 | −27.643 | 1.00 | 22.93 |
| ATOM | 457 | OD1 | ASN | A | 61 | −19.585 | 11.057 | −27.231 | 1.00 | 30.05 |
| ATOM | 458 | ND2 | ASN | A | 61 | −18.206 | 12.568 | −28.130 | 1.00 | 29.54 |
| ATOM | 459 | C | ASN | A | 61 | −18.480 | 8.659 | −28.907 | 1.00 | 17.28 |
| ATOM | 460 | O | ASN | A | 61 | −19.697 | 8.475 | −28.852 | 1.00 | 18.11 |
| ATOM | 461 | N | LEU | A | 62 | −17.799 | 8.647 | −30.056 | 1.00 | 16.54 |
| ATOM | 462 | CA | LEU | A | 62 | −18.460 | 8.379 | −31.334 | 1.00 | 16.19 |
| ATOM | 463 | CB | LEU | A | 62 | −17.479 | 8.572 | −32.499 | 1.00 | 16.85 |
| ATOM | 464 | CG | LEU | A | 62 | −17.047 | 10.027 | −32.697 | 1.00 | 18.47 |
| ATOM | 465 | CD1 | LEU | A | 62 | −16.118 | 10.153 | −33.916 | 1.00 | 20.38 |
| ATOM | 466 | CD2 | LEU | A | 62 | −18.263 | 10.925 | −32.837 | 1.00 | 19.93 |
| ATOM | 467 | C | LEU | A | 62 | −19.089 | 6.991 | −31.371 | 1.00 | 16.01 |
| ATOM | 468 | O | LEU | A | 62 | −20.225 | 6.833 | −31.842 | 1.00 | 15.98 |
| ATOM | 469 | N | ILE | A | 63 | −18.387 | 5.998 | −30.831 | 1.00 | 15.67 |
| ATOM | 470 | CA | ILE | A | 63 | −18.910 | 4.628 | −30.810 | 1.00 | 15.86 |
| ATOM | 471 | CB | ILE | A | 63 | −17.803 | 3.610 | −30.372 | 1.00 | 15.88 |
| ATOM | 472 | CG1 | ILE | A | 63 | −16.756 | 3.466 | −31.478 | 1.00 | 14.98 |
| ATOM | 473 | CD1 | ILE | A | 63 | −15.375 | 2.976 | −30.966 | 1.00 | 15.62 |
| ATOM | 474 | CG2 | ILE | A | 63 | −18.398 | 2.251 | −30.016 | 1.00 | 15.96 |
| ATOM | 475 | C | ILE | A | 63 | −20.156 | 4.538 | −29.920 | 1.00 | 16.39 |
| ATOM | 476 | O | ILE | A | 63 | −21.137 | 3.854 | −30.272 | 1.00 | 16.90 |
| ATOM | 477 | N | ASP | A | 64 | −20.129 | 5.242 | −28.796 | 1.00 | 16.51 |
| ATOM | 478 | CA | ASP | A | 64 | −21.299 | 5.324 | −27.922 | 1.00 | 17.76 |
| ATOM | 479 | CB | ASP | A | 64 | −20.953 | 6.022 | −26.594 | 1.00 | 17.81 |
| ATOM | 480 | CG | ASP | A | 64 | −20.089 | 5.164 | −25.682 | 1.00 | 17.84 |
| ATOM | 481 | OD1 | ASP | A | 64 | −19.883 | 3.944 | −25.963 | 1.00 | 18.57 |
| ATOM | 482 | OD2 | ASP | A | 64 | −19.595 | 5.699 | −24.659 | 1.00 | 20.95 |
| ATOM | 483 | C | ASP | A | 64 | −22.492 | 5.982 | −28.617 | 1.00 | 18.64 |
| ATOM | 484 | O | ASP | A | 64 | −23.617 | 5.493 | −28.507 | 1.00 | 20.85 |
| ATOM | 485 | N | ARG | A | 65 | −22.262 | 7.070 | −29.348 | 1.00 | 19.57 |
| ATOM | 486 | CA | ARG | A | 65 | −23.341 | 7.750 | −30.097 | 1.00 | 20.59 |
| ATOM | 487 | CB | ARG | A | 65 | −22.823 | 9.046 | −30.733 | 1.00 | 20.62 |
| ATOM | 488 | CG | ARG | A | 65 | −22.465 | 10.083 | −29.693 | 1.00 | 25.00 |
| ATOM | 489 | CD | ARG | A | 65 | −22.010 | 11.385 | −30.324 | 1.00 | 28.44 |
| ATOM | 490 | NE | ARG | A | 65 | −23.106 | 12.071 | −30.990 | 1.00 | 31.14 |
| ATOM | 491 | CZ | ARG | A | 65 | −23.968 | 12.878 | −30.373 | 1.00 | 32.75 |
| ATOM | 492 | NH1 | ARG | A | 65 | −23.873 | 13.095 | −29.060 | 1.00 | 32.10 |
| ATOM | 493 | NH2 | ARG | A | 65 | −24.928 | 13.459 | −31.080 | 1.00 | 32.31 |
| ATOM | 494 | C | ARG | A | 65 | −23.907 | 6.841 | −31.184 | 1.00 | 20.83 |
| ATOM | 495 | O | ARG | A | 65 | −25.129 | 6.711 | −31.357 | 1.00 | 20.48 |
| ATOM | 496 | N | PHE | A | 66 | −22.998 | 6.213 | −31.910 | 1.00 | 20.52 |
| ATOM | 497 | CA | PHE | A | 66 | −23.340 | 5.271 | −32.966 | 1.00 | 21.42 |
| ATOM | 498 | CB | PHE | A | 66 | −22.046 | 4.778 | −33.604 | 1.00 | 21.97 |
| ATOM | 499 | CG | PHE | A | 66 | −22.224 | 3.603 | −34.520 | 1.00 | 21.97 |
| ATOM | 500 | CD1 | PHE | A | 66 | −22.601 | 3.791 | −35.844 | 1.00 | 23.23 |
| ATOM | 501 | CE1 | PHE | A | 66 | −22.768 | 2.690 | −36.699 | 1.00 | 22.87 |
| ATOM | 502 | CZ | PHE | A | 66 | −22.552 | 1.409 | −36.221 | 1.00 | 22.60 |
| ATOM | 503 | CE2 | PHE | A | 66 | −22.165 | 1.216 | −34.895 | 1.00 | 23.74 |
| ATOM | 504 | CD2 | PHE | A | 66 | −22.006 | 2.309 | −34.054 | 1.00 | 23.07 |
| ATOM | 505 | C | PHE | A | 66 | −24.152 | 4.084 | −32.415 | 1.00 | 21.98 |
| ATOM | 506 | O | PHE | A | 66 | −25.040 | 3.551 | −33.099 | 1.00 | 21.80 |
| ATOM | 507 | N | THR | A | 67 | −23.831 | 3.654 | −31.195 | 1.00 | 22.48 |
| ATOM | 508 | CA | THR | A | 67 | −24.546 | 2.537 | −30.576 | 1.00 | 23.79 |
| ATOM | 509 | CB | THR | A | 67 | −23.809 | 1.999 | −29.333 | 1.00 | 23.68 |
| ATOM | 510 | OG1 | THR | A | 67 | −22.551 | 1.439 | −29.745 | 1.00 | 23.93 |
| ATOM | 511 | CG2 | THR | A | 67 | −24.613 | 0.881 | −28.653 | 1.00 | 23.90 |
| ATOM | 512 | C | THR | A | 67 | −25.992 | 2.925 | −30.258 | 1.00 | 24.77 |
| ATOM | 513 | O | THR | A | 67 | −26.893 | 2.090 | −30.349 | 1.00 | 25.31 |
| ATOM | 514 | N | GLU | A | 68 | −26.207 | 4.189 | −29.916 | 1.00 | 25.62 |
| ATOM | 515 | CA | GLU | A | 68 | −27.540 | 4.688 | −29.616 | 1.00 | 27.41 |
| ATOM | 516 | CB | GLU | A | 68 | −27.466 | 6.038 | −28.894 | 1.00 | 28.13 |
| ATOM | 517 | CG | GLU | A | 68 | −26.997 | 5.951 | −27.446 | 1.00 | 32.86 |
| ATOM | 518 | CD | GLU | A | 68 | −28.095 | 5.487 | −26.468 | 1.00 | 38.33 |
| ATOM | 519 | OE1 | GLU | A | 68 | −29.241 | 5.982 | −26.542 | 1.00 | 40.42 |
| ATOM | 520 | OE2 | GLU | A | 68 | −27.799 | 4.633 | −25.607 | 1.00 | 42.48 |
| ATOM | 521 | C | GLU | A | 68 | −28.418 | 4.784 | −30.873 | 1.00 | 27.86 |
| ATOM | 522 | O | GLU | A | 68 | −29.602 | 4.429 | −30.845 | 1.00 | 28.00 |
| ATOM | 523 | N | THR | A | 69 | −27.833 | 5.260 | −31.968 | 1.00 | 27.32 |
| ATOM | 524 | CA | THR | A | 69 | −28.540 | 5.373 | −33.241 | 1.00 | 27.32 |
| ATOM | 525 | CB | THR | A | 69 | −29.113 | 6.792 | −33.451 | 1.00 | 27.49 |
| ATOM | 526 | OG1 | THR | A | 69 | −29.922 | 7.153 | −32.334 | 1.00 | 30.86 |
| ATOM | 527 | CG2 | THR | A | 69 | −29.945 | 6.843 | −34.719 | 1.00 | 29.06 |
| ATOM | 528 | C | THR | A | 69 | −27.563 | 5.133 | −34.359 | 1.00 | 26.07 |
| ATOM | 529 | O | THR | A | 69 | −26.619 | 5.905 | −34.523 | 1.00 | 25.25 |
| ATOM | 530 | N | TYR | A | 70 | −27.790 | 4.064 | −35.123 | 1.00 | 25.65 |
| ATOM | 531 | CA | TYR | A | 70 | −26.948 | 3.738 | −36.267 | 1.00 | 25.36 |
| ATOM | 532 | CB | TYR | A | 70 | −27.480 | 2.504 | −37.013 | 1.00 | 25.26 |
| ATOM | 533 | CG | TYR | A | 70 | −26.638 | 2.104 | −38.217 | 1.00 | 25.62 |
| ATOM | 534 | CD1 | TYR | A | 70 | −26.949 | 2.567 | −39.498 | 1.00 | 25.62 |
| ATOM | 535 | CE1 | TYR | A | 70 | −26.190 | 2.201 | −40.611 | 1.00 | 26.48 |
| ATOM | 536 | CZ | TYR | A | 70 | −25.099 | 1.360 | −40.437 | 1.00 | 25.36 |
| ATOM | 537 | OH | TYR | A | 70 | −24.354 | 0.995 | −41.520 | 1.00 | 24.80 |
| ATOM | 538 | CE2 | TYR | A | 70 | −24.770 | 0.883 | −39.175 | 1.00 | 25.59 |
| ATOM | 539 | CD2 | TYR | A | 70 | −25.538 | 1.259 | −38.071 | 1.00 | 25.36 |
| ATOM | 540 | C | TYR | A | 70 | −26.816 | 4.909 | −37.230 | 1.00 | 25.38 |
| ATOM | 541 | O | TYR | A | 70 | −27.802 | 5.573 | −37.583 | 1.00 | 24.87 |
| ATOM | 542 | N | ASP | A | 71 | −25.575 | 5.127 | −37.666 | 1.00 | 25.16 |
| ATOM | 543 | CA | ASP | A | 71 | −25.188 | 6.210 | −38.550 | 1.00 | 25.40 |
| ATOM | 544 | CB | ASP | A | 71 | −24.668 | 7.404 | −37.724 | 1.00 | 25.60 |
| ATOM | 545 | CG | ASP | A | 71 | −24.361 | 8.642 | −38.573 | 1.00 | 26.17 |
| ATOM | 546 | OD1 | ASP | A | 71 | −23.801 | 8.526 | −39.681 | 1.00 | 25.82 |
| ATOM | 547 | OD2 | ASP | A | 71 | −24.675 | 9.755 | −38.108 | 1.00 | 27.85 |
| ATOM | 548 | C | ASP | A | 71 | −24.061 | 5.619 | −39.386 | 1.00 | 25.64 |
| ATOM | 549 | O | ASP | A | 71 | −22.956 | 5.377 | −38.875 | 1.00 | 24.82 |
| ATOM | 550 | N | ALA | A | 72 | −24.347 | 5.379 | −40.665 | 1.00 | 24.95 |
| ATOM | 551 | CA | ALA | A | 72 | −23.380 | 4.764 | −41.586 | 1.00 | 24.24 |
| ATOM | 552 | CB | ALA | A | 72 | −24.047 | 4.434 | −42.921 | 1.00 | 24.35 |
| ATOM | 553 | C | ALA | A | 72 | −22.152 | 5.657 | −41.812 | 1.00 | 24.04 |
| ATOM | 554 | O | ALA | A | 72 | −21.054 | 5.159 | −42.086 | 1.00 | 23.21 |
| ATOM | 555 | N | GLY | A | 73 | −22.356 | 6.970 | −41.695 | 1.00 | 23.63 |
| ATOM | 556 | CA | GLY | A | 73 | −21.265 | 7.938 | −41.761 | 1.00 | 24.20 |
| ATOM | 557 | C | GLY | A | 73 | −20.285 | 7.809 | −40.596 | 1.00 | 23.85 |
| ATOM | 558 | O | GLY | A | 73 | −19.067 | 7.927 | −40.806 | 1.00 | 24.81 |
| ATOM | 559 | N | LEU | A | 74 | −20.798 | 7.588 | −39.376 | 1.00 | 22.78 |
| ATOM | 560 | CA | LEU | A | 74 | −19.927 | 7.347 | −38.232 | 1.00 | 22.21 |
| ATOM | 561 | CB | LEU | A | 74 | −20.662 | 7.449 | −36.879 | 1.00 | 22.58 |
| ATOM | 562 | CG | LEU | A | 74 | −21.132 | 8.846 | −36.434 | 1.00 | 24.06 |
| ATOM | 563 | CD1 | LEU | A | 74 | −21.732 | 8.793 | −35.019 | 1.00 | 22.31 |
| ATOM | 564 | CD2 | LEU | A | 74 | −20.002 | 9.869 | −36.503 | 1.00 | 26.04 |
| ATOM | 565 | C | LEU | A | 74 | −19.256 | 5.999 | −38.370 | 1.00 | 21.94 |
| ATOM | 566 | O | LEU | A | 74 | −18.060 | 5.872 | −38.098 | 1.00 | 20.62 |
| ATOM | 567 | N | GLN | A | 75 | −20.019 | 4.988 | −38.814 | 1.00 | 21.47 |
| ATOM | 568 | CA | GLN | A | 75 | −19.451 | 3.654 | −38.989 | 1.00 | 21.07 |
| ATOM | 569 | CB | GLN | A | 75 | −20.469 | 2.709 | −39.619 | 1.00 | 21.44 |
| ATOM | 570 | CG | GLN | A | 75 | −20.002 | 1.280 | −39.594 | 1.00 | 23.11 |
| ATOM | 571 | CD | GLN | A | 75 | −21.101 | 0.312 | −39.945 | 1.00 | 24.74 |
| ATOM | 572 | OE1 | GLN | A | 75 | −21.273 | −0.719 | −39.290 | 1.00 | 26.57 |
| ATOM | 573 | NE2 | GLN | A | 75 | −21.872 | 0.654 | −40.950 | 1.00 | 23.71 |
| ATOM | 574 | C | GLN | A | 75 | −18.219 | 3.704 | −39.889 | 1.00 | 21.05 |
| ATOM | 575 | O | GLN | A | 75 | −17.229 | 3.046 | −39.611 | 1.00 | 21.10 |
| ATOM | 576 | N | ARG | A | 76 | −18.294 | 4.466 | −40.975 | 1.00 | 20.73 |
| ATOM | 577 | CA | ARG | A | 76 | −17.184 | 4.551 | −41.910 | 1.00 | 21.56 |
| ATOM | 578 | CB | ARG | A | 76 | −17.544 | 5.460 | −43.101 | 1.00 | 22.06 |
| ATOM | 579 | CG | ARG | A | 76 | −16.452 | 5.627 | −44.168 | 1.00 | 24.00 |
| ATOM | 580 | CD | ARG | A | 76 | −15.586 | 6.895 | −43.934 | 1.00 | 27.60 |
| ATOM | 581 | NE | ARG | A | 76 | −16.275 | 8.150 | −44.280 | 1.00 | 30.93 |
| ATOM | 582 | CZ | ARG | A | 76 | −15.778 | 9.378 | −44.082 | 1.00 | 32.20 |
| ATOM | 583 | NH1 | ARG | A | 76 | −14.572 | 9.556 | −43.535 | 1.00 | 30.72 |
| ATOM | 584 | NH2 | ARG | A | 76 | −16.491 | 10.443 | −44.437 | 1.00 | 32.29 |
| ATOM | 585 | C | ARG | A | 76 | −15.942 | 5.063 | −41.162 | 1.00 | 20.85 |
| ATOM | 586 | O | ARG | A | 76 | −14.858 | 4.514 | −41.296 | 1.00 | 20.93 |
| ATOM | 587 | N | ARG | A | 77 | −16.116 | 6.119 | −40.378 | 1.00 | 20.17 |
| ATOM | 588 | CA | ARG | A | 77 | −14.990 | 6.723 | −39.631 | 1.00 | 19.61 |
| ATOM | 589 | CB | ARG | A | 77 | −15.419 | 8.058 | −39.009 | 1.00 | 19.11 |
| ATOM | 590 | CG | ARG | A | 77 | −15.719 | 9.106 | −40.075 | 1.00 | 20.18 |
| ATOM | 591 | CD | ARG | A | 77 | −16.379 | 10.299 | −39.459 | 1.00 | 22.42 |
| ATOM | 592 | NE | ARG | A | 77 | −16.489 | 11.411 | −40.396 | 1.00 | 24.06 |
| ATOM | 593 | CZ | ARG | A | 77 | −17.501 | 11.592 | −41.243 | 1.00 | 27.81 |
| ATOM | 594 | NH1 | ARG | A | 77 | −18.508 | 10.714 | −41.303 | 1.00 | 28.01 |
| ATOM | 595 | NH2 | ARG | A | 77 | −17.509 | 12.658 | −42.033 | 1.00 | 27.19 |
| ATOM | 596 | C | ARG | A | 77 | −14.425 | 5.789 | −38.570 | 1.00 | 18.77 |
| ATOM | 597 | O | ARG | A | 77 | −13.197 | 5.685 | −38.411 | 1.00 | 18.64 |
| ATOM | 598 | N | ILE | A | 78 | −15.320 | 5.117 | −37.852 | 1.00 | 17.79 |
| ATOM | 599 | CA | ILE | A | 78 | −14.931 | 4.125 | −36.857 | 1.00 | 18.13 |
| ATOM | 600 | CB | ILE | A | 78 | −16.165 | 3.514 | −36.151 | 1.00 | 17.88 |
| ATOM | 601 | CG1 | ILE | A | 78 | −16.862 | 4.564 | −35.282 | 1.00 | 18.78 |
| ATOM | 602 | CD1 | ILE | A | 78 | −18.274 | 4.154 | −34.879 | 1.00 | 19.16 |
| ATOM | 603 | CG2 | ILE | A | 78 | −15.772 | 2.279 | −35.308 | 1.00 | 18.15 |
| ATOM | 604 | C | ILE | A | 78 | −14.105 | 3.012 | −37.491 | 1.00 | 18.09 |
| ATOM | 605 | O | ILE | A | 78 | −13.088 | 2.612 | −36.949 | 1.00 | 17.42 |
| ATOM | 606 | N | GLU | A | 79 | −14.565 | 2.495 | −38.631 | 1.00 | 18.82 |
| ATOM | 607 | CA | GLU | A | 79 | −13.826 | 1.446 | −39.341 | 1.00 | 20.22 |
| ATOM | 608 | CB | GLU | A | 79 | −14.587 | 1.017 | −40.609 | 1.00 | 20.36 |
| ATOM | 609 | CG | GLU | A | 79 | −15.811 | 0.136 | −40.312 | 1.00 | 22.29 |
| ATOM | 610 | CD | GLU | A | 79 | −16.633 | −0.206 | −41.565 | 1.00 | 22.92 |
| ATOM | 611 | OE1 | GLU | A | 79 | −16.345 | 0.328 | −42.670 | 1.00 | 26.99 |
| ATOM | 612 | OE2 | GLU | A | 79 | −17.579 | −1.012 | −41.425 | 1.00 | 25.69 |
| ATOM | 613 | C | GLU | A | 79 | −12.418 | 1.918 | −39.704 | 1.00 | 18.80 |
| ATOM | 614 | O | GLU | A | 79 | −11.450 | 1.191 | −39.507 | 1.00 | 19.44 |
| ATOM | 615 | N | GLN | A | 80 | −12.301 | 3.147 | −40.211 | 1.00 | 18.84 |
| ATOM | 616 | CA | GLN | A | 80 | −10.998 | 3.661 | −40.636 | 1.00 | 17.90 |
| ATOM | 617 | CB | GLN | A | 80 | −11.149 | 4.921 | −41.482 | 1.00 | 18.92 |
| ATOM | 618 | CG | GLN | A | 80 | −11.794 | 4.593 | −42.844 | 1.00 | 21.99 |
| ATOM | 619 | CD | GLN | A | 80 | −12.040 | 5.799 | −43.707 | 1.00 | 27.48 |
| ATOM | 620 | OE1 | GLN | A | 80 | −12.265 | 6.898 | −43.223 | 1.00 | 30.45 |
| ATOM | 621 | NE2 | GLN | A | 80 | −12.037 | 5.586 | −45.013 | 1.00 | 32.63 |
| ATOM | 622 | C | GLN | A | 80 | −10.059 | 3.892 | −39.456 | 1.00 | 17.64 |
| ATOM | 623 | O | GLN | A | 80 | −8.862 | 3.612 | −39.535 | 1.00 | 17.21 |
| ATOM | 624 | N | TYR | A | 81 | −10.607 | 4.408 | −38.365 | 1.00 | 17.29 |
| ATOM | 625 | CA | TYR | A | 81 | −9.839 | 4.552 | −37.122 | 1.00 | 17.64 |
| ATOM | 626 | CB | TYR | A | 81 | −10.750 | 5.139 | −36.023 | 1.00 | 17.24 |
| ATOM | 627 | CG | TYR | A | 81 | −10.188 | 4.973 | −34.621 | 1.00 | 17.79 |
| ATOM | 628 | CD1 | TYR | A | 81 | −9.085 | 5.728 | −34.184 | 1.00 | 16.36 |
| ATOM | 629 | CE1 | TYR | A | 81 | −8.561 | 5.568 | −32.882 | 1.00 | 17.45 |
| ATOM | 630 | CZ | TYR | A | 81 | −9.146 | 4.646 | −32.009 | 1.00 | 16.35 |
| ATOM | 631 | OH | TYR | A | 81 | −8.654 | 4.457 | −30.724 | 1.00 | 17.06 |
| ATOM | 632 | CE2 | TYR | A | 81 | −10.238 | 3.890 | −32.423 | 1.00 | 17.51 |
| ATOM | 633 | CD2 | TYR | A | 81 | −10.754 | 4.055 | −33.729 | 1.00 | 17.00 |
| ATOM | 634 | C | TYR | A | 81 | −9.271 | 3.197 | −36.686 | 1.00 | 18.04 |
| ATOM | 635 | O | TYR | A | 81 | −8.098 | 3.083 | −36.321 | 1.00 | 17.85 |
| ATOM | 636 | N | ILE | A | 82 | −10.096 | 2.159 | −36.746 | 1.00 | 17.99 |
| ATOM | 637 | CA | ILE | A | 82 | −9.661 | 0.839 | −36.295 | 1.00 | 19.35 |
| ATOM | 638 | CB | ILE | A | 82 | −10.844 | −0.166 | −36.187 | 1.00 | 18.93 |
| ATOM | 639 | CG1 | ILE | A | 82 | −11.753 | 0.233 | −35.017 | 1.00 | 19.40 |
| ATOM | 640 | CD1 | ILE | A | 82 | −13.093 | −0.565 | −34.896 | 1.00 | 20.46 |
| ATOM | 641 | CG2 | ILE | A | 82 | −10.301 | −1.587 | −35.984 | 1.00 | 20.61 |
| ATOM | 642 | C | ILE | A | 82 | −8.547 | 0.292 | −37.194 | 1.00 | 19.92 |
| ATOM | 643 | O | ILE | A | 82 | −7.543 | −0.239 | −36.708 | 1.00 | 20.26 |
| ATOM | 644 | N | THR | A | 83 | −8.713 | 0.432 | −38.503 | 1.00 | 20.05 |
| ATOM | 645 | CA | THR | A | 83 | −7.709 | −0.100 | −39.406 | 1.00 | 21.11 |
| ATOM | 646 | CB | THR | A | 83 | −8.241 | −0.297 | −40.845 | 1.00 | 21.63 |
| ATOM | 647 | OG1 | THR | A | 83 | −8.830 | 0.902 | −41.306 | 1.00 | 25.88 |
| ATOM | 648 | CG2 | THR | A | 83 | −9.330 | −1.347 | −40.851 | 1.00 | 21.56 |
| ATOM | 649 | C | THR | A | 83 | −6.410 | 0.690 | −39.337 | 1.00 | 20.59 |
| ATOM | 650 | O | THR | A | 83 | −5.338 | 0.105 | −39.511 | 1.00 | 20.72 |
| ATOM | 651 | N | ALA | A | 84 | −6.494 | 1.997 | −39.050 | 1.00 | 19.51 |
| ATOM | 652 | CA | ALA | A | 84 | −5.292 | 2.809 | −38.777 | 1.00 | 19.37 |
| ATOM | 653 | CB | ALA | A | 84 | −5.652 | 4.290 | −38.507 | 1.00 | 19.42 |
| ATOM | 654 | C | ALA | A | 84 | −4.436 | 2.231 | −37.643 | 1.00 | 19.32 |
| ATOM | 655 | O | ALA | A | 84 | −3.208 | 2.370 | −37.649 | 1.00 | 19.47 |
| ATOM | 656 | N | GLN | A | 85 | −5.063 | 1.535 | −36.695 | 1.00 | 19.34 |
| ATOM | 657 | CA | GLN | A | 85 | −4.325 | 0.998 | −35.544 | 1.00 | 18.78 |
| ATOM | 658 | CB | GLN | A | 85 | −5.266 | 0.609 | −34.396 | 1.00 | 19.29 |
| ATOM | 659 | CG | GLN | A | 85 | −6.260 | 1.735 | −34.007 | 1.00 | 17.98 |
| ATOM | 660 | CD | GLN | A | 85 | −5.593 | 3.098 | −33.830 | 1.00 | 17.20 |
| ATOM | 661 | OE1 | GLN | A | 85 | −6.021 | 4.095 | −34.418 | 1.00 | 20.82 |
| ATOM | 662 | NE2 | GLN | A | 85 | −4.540 | 3.143 | −33.034 | 1.00 | 13.47 |
| ATOM | 663 | C | GLN | A | 85 | −3.447 | −0.178 | −35.932 | 1.00 | 19.08 |
| ATOM | 664 | O | GLN | A | 85 | −2.478 | −0.473 | −35.251 | 1.00 | 17.94 |
| ATOM | 665 | N | VAL | A | 86 | −3.808 | −0.838 | −37.032 | 1.00 | 19.32 |
| ATOM | 666 | CA | VAL | A | 86 | −2.999 | −1.928 | −37.588 | 1.00 | 20.79 |
| ATOM | 667 | CB | VAL | A | 86 | −3.670 | −2.581 | −38.823 | 1.00 | 21.18 |
| ATOM | 668 | CG1 | VAL | A | 86 | −2.712 | −3.615 | −39.454 | 1.00 | 22.66 |
| ATOM | 669 | CG2 | VAL | A | 86 | −4.980 | −3.250 | −38.400 | 1.00 | 21.47 |
| ATOM | 670 | C | VAL | A | 86 | −1.617 | −1.381 | −37.940 | 1.00 | 20.63 |
| ATOM | 671 | O | VAL | A | 86 | −0.602 | −1.930 | −37.520 | 1.00 | 21.41 |
| ATOM | 672 | N | THR | A | 87 | −1.604 | −0.251 | −38.641 | 1.00 | 20.67 |
| ATOM | 673 | CA | THR | A | 87 | −0.361 | 0.419 | −39.015 | 1.00 | 21.10 |
| ATOM | 674 | CB | THR | A | 87 | −0.659 | 1.583 | −39.986 | 1.00 | 21.59 |
| ATOM | 675 | OG1 | THR | A | 87 | −1.176 | 1.033 | −41.202 | 1.00 | 23.63 |
| ATOM | 676 | CG2 | THR | A | 87 | 0.585 | 2.370 | −40.305 | 1.00 | 22.02 |
| ATOM | 677 | C | THR | A | 87 | 0.412 | 0.881 | −37.795 | 1.00 | 20.28 |
| ATOM | 678 | O | THR | A | 87 | 1.620 | 0.641 | −37.679 | 1.00 | 20.22 |
| ATOM | 679 | N | LEU | A | 88 | −0.280 | 1.543 | −36.874 | 1.00 | 19.11 |
| ATOM | 680 | CA | LEU | A | 88 | 0.367 | 2.097 | −35.697 | 1.00 | 18.71 |
| ATOM | 681 | CB | LEU | A | 88 | −0.585 | 3.015 | −34.903 | 1.00 | 18.08 |
| ATOM | 682 | CG | LEU | A | 88 | −1.016 | 4.294 | −35.596 | 1.00 | 18.50 |
| ATOM | 683 | CD1 | LEU | A | 88 | −2.038 | 5.058 | −34.706 | 1.00 | 19.12 |
| ATOM | 684 | CD2 | LEU | A | 88 | 0.206 | 5.193 | −35.937 | 1.00 | 19.81 |
| ATOM | 685 | C | LEU | A | 88 | 0.976 | 1.057 | −34.780 | 1.00 | 18.33 |
| ATOM | 686 | O | LEU | A | 88 | 2.101 | 1.244 | −34.336 | 1.00 | 18.76 |
| ATOM | 687 | N | GLN | A | 89 | 0.255 | −0.029 | −34.492 | 1.00 | 18.75 |
| ATOM | 688 | CA | GLN | A | 89 | 0.809 | −1.085 | −33.623 | 1.00 | 19.67 |
| ATOM | 689 | CB | GLN | A | 89 | −0.199 | −2.201 | −33.373 | 1.00 | 19.69 |
| ATOM | 690 | CG | GLN | A | 89 | −1.397 | −1.775 | −32.564 | 1.00 | 19.25 |
| ATOM | 691 | CD | GLN | A | 89 | −2.140 | −2.951 | −32.004 | 1.00 | 20.83 |
| ATOM | 692 | OE1 | GLN | A | 89 | −2.121 | −4.037 | −32.580 | 1.00 | 19.26 |
| ATOM | 693 | NE2 | GLN | A | 89 | −2.802 | −2.751 | −30.861 | 1.00 | 19.61 |
| ATOM | 694 | C | GLN | A | 89 | 2.097 | −1.683 | −34.203 | 1.00 | 20.68 |
| ATOM | 695 | O | GLN | A | 89 | 3.013 | −2.026 | −33.461 | 1.00 | 21.21 |
| ATOM | 696 | N | GLY | A | 90 | 2.164 | −1.778 | −35.524 | 1.00 | 21.75 |
| ATOM | 697 | CA | GLY | A | 90 | 3.330 | −2.374 | −36.173 | 1.00 | 23.35 |
| ATOM | 698 | C | GLY | A | 90 | 4.604 | −1.552 | −36.096 | 1.00 | 24.79 |
| ATOM | 699 | O | GLY | A | 90 | 5.699 | −2.104 | −36.299 | 1.00 | 25.52 |
| ATOM | 700 | N | ASN | A | 91 | 4.477 | −0.252 | −35.796 | 1.00 | 25.17 |
| ATOM | 701 | CA | ASN | A | 91 | 5.596 | 0.714 | −35.870 | 1.00 | 26.14 |
| ATOM | 702 | CB | ASN | A | 91 | 5.108 | 2.161 | −35.653 | 1.00 | 26.83 |
| ATOM | 703 | CG | ASN | A | 91 | 4.615 | 2.849 | −36.919 | 1.00 | 29.67 |
| ATOM | 704 | OD1 | ASN | A | 91 | 4.869 | 2.414 | −38.051 | 1.00 | 34.53 |
| ATOM | 705 | ND2 | ASN | A | 91 | 3.927 | 3.981 | −36.724 | 1.00 | 32.76 |
| ATOM | 706 | C | ASN | A | 91 | 6.656 | 0.489 | −34.820 | 1.00 | 25.67 |
| ATOM | 707 | O | ASN | A | 91 | 6.346 | 0.385 | −33.644 | 1.00 | 25.70 |
| ATOM | 708 | N | SER | A | 92 | 7.918 | 0.472 | −35.227 | 1.00 | 25.32 |
| ATOM | 709 | CA | SER | A | 92 | 8.990 | 0.668 | −34.257 | 1.00 | 25.27 |
| ATOM | 710 | CB | SER | A | 92 | 10.314 | 0.107 | −34.775 | 1.00 | 26.03 |
| ATOM | 711 | OG | SER | A | 92 | 10.212 | −1.305 | −34.803 | 1.00 | 30.67 |
| ATOM | 712 | C | SER | A | 92 | 9.103 | 2.171 | −34.003 | 1.00 | 23.80 |
| ATOM | 713 | O | SER | A | 92 | 9.055 | 2.979 | −34.942 | 1.00 | 24.56 |
| ATOM | 714 | N | ASN | A | 93 | 9.246 | 2.544 | −32.743 | 1.00 | 22.30 |
| ATOM | 715 | CA | ASN | A | 93 | 9.236 | 3.953 | −32.383 | 1.00 | 21.23 |
| ATOM | 716 | CB | ASN | A | 93 | 7.769 | 4.423 | −32.201 | 1.00 | 21.41 |
| ATOM | 717 | CG | ASN | A | 93 | 7.075 | 3.704 | −31.051 | 1.00 | 19.23 |
| ATOM | 718 | OD1 | ASN | A | 93 | 7.564 | 3.736 | −29.927 | 1.00 | 17.81 |
| ATOM | 719 | ND2 | ASN | A | 93 | 5.974 | 3.024 | −31.335 | 1.00 | 19.43 |
| ATOM | 720 | C | ASN | A | 93 | 10.103 | 4.149 | −31.150 | 1.00 | 20.39 |
| ATOM | 721 | O | ASN | A | 93 | 10.625 | 3.154 | −30.607 | 1.00 | 19.46 |
| ATOM | 722 | N | PRO | A | 94 | 10.337 | 5.410 | −30.732 | 1.00 | 19.95 |
| ATOM | 723 | CA | PRO | A | 94 | 11.228 | 5.604 | −29.574 | 1.00 | 19.89 |
| ATOM | 724 | CB | PRO | A | 94 | 11.235 | 7.131 | −29.385 | 1.00 | 19.51 |
| ATOM | 725 | CG | PRO | A | 94 | 10.988 | 7.653 | −30.753 | 1.00 | 19.96 |
| ATOM | 726 | CD | PRO | A | 94 | 9.952 | 6.717 | −31.325 | 1.00 | 19.99 |
| ATOM | 727 | C | PRO | A | 94 | 10.870 | 4.898 | −28.263 | 1.00 | 20.64 |
| ATOM | 728 | O | PRO | A | 94 | 11.756 | 4.727 | −27.430 | 1.00 | 20.54 |
| ATOM | 729 | N | SER | A | 95 | 9.610 | 4.485 | −28.073 | 1.00 | 20.36 |
| ATOM | 730 | CA | SER | A | 95 | 9.264 | 3.674 | −26.902 | 1.00 | 21.00 |
| ATOM | 731 | CB | SER | A | 95 | 7.770 | 3.736 | −26.587 | 1.00 | 20.05 |
| ATOM | 732 | OG | SER | A | 95 | 7.413 | 5.036 | −26.147 | 1.00 | 19.97 |
| ATOM | 733 | C | SER | A | 95 | 9.679 | 2.204 | −27.066 | 1.00 | 21.73 |
| ATOM | 734 | O | SER | A | 95 | 9.809 | 1.499 | −26.072 | 1.00 | 22.10 |
| ATOM | 735 | N | GLY | A | 96 | 9.853 | 1.748 | −28.306 | 1.00 | 21.90 |
| ATOM | 736 | CA | GLY | A | 96 | 10.229 | 0.350 | −28.562 | 1.00 | 23.56 |
| ATOM | 737 | C | GLY | A | 96 | 9.506 | −0.196 | −29.774 | 1.00 | 24.14 |
| ATOM | 738 | O | GLY | A | 96 | 9.121 | 0.557 | −30.664 | 1.00 | 24.24 |
| ATOM | 739 | N | SER | A | 97 | 9.315 | −1.510 | −29.828 | 1.00 | 25.24 |
| ATOM | 740 | CA | SER | A | 97 | 8.703 | −2.116 | −31.000 | 1.00 | 25.77 |
| ATOM | 741 | CB | SER | A | 97 | 9.751 | −2.874 | −31.834 | 1.00 | 27.15 |
| ATOM | 742 | OG | SER | A | 97 | 10.120 | −4.086 | −31.189 | 1.00 | 30.57 |
| ATOM | 743 | C | SER | A | 97 | 7.590 | −3.042 | −30.571 | 1.00 | 25.27 |
| ATOM | 744 | O | SER | A | 97 | 7.346 | −3.199 | −29.376 | 1.00 | 24.85 |
| ATOM | 745 | N | LEU | A | 98 | 6.930 | −3.655 | −31.543 | 1.00 | 24.82 |
| ATOM | 746 | CA | LEU | A | 98 | 5.826 | −4.559 | −31.252 | 1.00 | 25.88 |
| ATOM | 747 | CB | LEU | A | 98 | 4.982 | −4.813 | −32.504 | 1.00 | 25.31 |
| ATOM | 748 | CG | LEU | A | 98 | 3.714 | −5.673 | −32.420 | 1.00 | 25.89 |
| ATOM | 749 | CD1 | LEU | A | 98 | 2.745 | −5.169 | −31.337 | 1.00 | 25.58 |
| ATOM | 750 | CD2 | LEU | A | 98 | 3.006 | −5.724 | −33.778 | 1.00 | 26.27 |
| ATOM | 751 | C | LEU | A | 98 | 6.310 | −5.866 | −30.604 | 1.00 | 26.75 |
| ATOM | 752 | O | LEU | A | 98 | 5.607 | −6.438 | −29.762 | 1.00 | 27.41 |
| ATOM | 753 | N | ALA | A | 99 | 7.528 | −6.290 | −30.950 | 1.00 | 27.23 |
| ATOM | 754 | CA | ALA | A | 99 | 8.074 | −7.590 | −30.533 | 1.00 | 27.89 |
| ATOM | 755 | CB | ALA | A | 99 | 9.566 | −7.700 | −30.935 | 1.00 | 27.68 |
| ATOM | 756 | C | ALA | A | 99 | 7.893 | −7.911 | −29.053 | 1.00 | 27.86 |
| ATOM | 757 | O | ALA | A | 99 | 7.450 | −9.007 | −28.711 | 1.00 | 28.77 |
| ATOM | 758 | N | ASP | A | 100 | 8.241 | −6.966 | −28.181 | 1.00 | 27.75 |
| ATOM | 759 | CA | ASP | A | 100 | 8.030 | −7.137 | −26.741 | 1.00 | 27.20 |
| ATOM | 760 | CB | ASP | A | 100 | 9.328 | −6.937 | −25.966 | 1.00 | 27.10 |
| ATOM | 761 | CG | ASP | A | 100 | 9.845 | −5.525 | −26.038 | 1.00 | 30.19 |
| ATOM | 762 | OD1 | ASP | A | 100 | 10.891 | −5.281 | −25.419 | 1.00 | 32.28 |
| ATOM | 763 | OD2 | ASP | A | 100 | 9.225 | −4.654 | −26.694 | 1.00 | 30.36 |
| ATOM | 764 | C | ASP | A | 100 | 6.905 | −6.256 | −26.173 | 1.00 | 25.74 |
| ATOM | 765 | O | ASP | A | 100 | 6.761 | −6.108 | −24.956 | 1.00 | 26.33 |
| ATOM | 766 | N | GLY | A | 101 | 6.118 | −5.683 | −27.075 | 1.00 | 24.93 |
| ATOM | 767 | CA | GLY | A | 101 | 4.982 | −4.853 | −26.707 | 1.00 | 23.22 |
| ATOM | 768 | C | GLY | A | 101 | 5.326 | −3.418 | −26.342 | 1.00 | 22.68 |
| ATOM | 769 | O | GLY | A | 101 | 4.419 | −2.580 | −26.287 | 1.00 | 21.48 |
| ATOM | 770 | N | SER | A | 102 | 6.609 | −3.117 | −26.126 | 1.00 | 21.53 |
| ATOM | 771 | CA | SER | A | 102 | 6.996 | −1.815 | −25.563 | 1.00 | 21.49 |
| ATOM | 772 | CB | SER | A | 102 | 8.483 | −1.739 | −25.199 | 1.00 | 22.07 |
| ATOM | 773 | OG | SER | A | 102 | 9.283 | −1.958 | −26.345 | 1.00 | 21.77 |
| ATOM | 774 | C | SER | A | 102 | 6.604 | −0.643 | −26.449 | 1.00 | 20.66 |
| ATOM | 775 | O | SER | A | 102 | 6.279 | 0.403 | −25.925 | 1.00 | 20.67 |
| ATOM | 776 | N | GLY | A | 103 | 6.636 | −0.819 | −27.771 | 1.00 | 19.97 |
| ATOM | 777 | CA | GLY | A | 103 | 6.257 | 0.255 | −28.707 | 1.00 | 19.46 |
| ATOM | 778 | C | GLY | A | 103 | 4.824 | 0.777 | −28.539 | 1.00 | 18.94 |
| ATOM | 779 | O | GLY | A | 103 | 4.525 | 1.903 | −28.945 | 1.00 | 18.13 |
| ATOM | 780 | N | LEU | A | 104 | 3.939 | −0.043 | −27.956 | 1.00 | 18.13 |
| ATOM | 781 | CA | LEU | A | 104 | 2.517 | 0.326 | −27.818 | 1.00 | 16.92 |
| ATOM | 782 | CB | LEU | A | 104 | 1.672 | −0.924 | −27.447 | 1.00 | 17.28 |
| ATOM | 783 | CG | LEU | A | 104 | 1.715 | −2.104 | −28.430 | 1.00 | 16.76 |
| ATOM | 784 | CD1 | LEU | A | 104 | 1.072 | −3.356 | −27.836 | 1.00 | 21.62 |
| ATOM | 785 | CD2 | LEU | A | 104 | 1.069 | −1.751 | −29.761 | 1.00 | 19.06 |
| ATOM | 786 | C | LEU | A | 104 | 2.283 | 1.464 | −26.798 | 1.00 | 16.57 |
| ATOM | 787 | O | LEU | A | 104 | 1.202 | 2.092 | −26.807 | 1.00 | 16.79 |
| ATOM | 788 | N | GLY | A | 105 | 3.279 | 1.713 | −25.936 | 1.00 | 14.86 |
| ATOM | 789 | CA | GLY | A | 105 | 3.255 | 2.802 | −24.938 | 1.00 | 15.94 |
| ATOM | 790 | C | GLY | A | 105 | 3.558 | 4.199 | −25.482 | 1.00 | 15.05 |
| ATOM | 791 | O | GLY | A | 105 | 3.481 | 5.179 | −24.755 | 1.00 | 15.93 |
| ATOM | 792 | N | GLU | A | 106 | 3.869 | 4.292 | −26.780 | 1.00 | 14.63 |
| ATOM | 793 | CA | GLU | A | 106 | 4.236 | 5.548 | −27.416 | 1.00 | 14.49 |
| ATOM | 794 | CB | GLU | A | 106 | 4.728 | 5.250 | −28.847 | 1.00 | 13.83 |
| ATOM | 795 | CG | GLU | A | 106 | 5.215 | 6.470 | −29.678 | 1.00 | 16.14 |
| ATOM | 796 | CD | GLU | A | 106 | 6.479 | 7.155 | −29.139 | 1.00 | 18.44 |
| ATOM | 797 | OE1 | GLU | A | 106 | 6.978 | 6.817 | −28.044 | 1.00 | 21.44 |
| ATOM | 798 | OE2 | GLU | A | 106 | 6.972 | 8.083 | −29.817 | 1.00 | 21.04 |
| ATOM | 799 | C | GLU | A | 106 | 3.012 | 6.484 | −27.413 | 1.00 | 14.32 |
| ATOM | 800 | O | GLU | A | 106 | 1.928 | 6.074 | −27.828 | 1.00 | 15.51 |
| ATOM | 801 | N | PRO | A | 107 | 3.164 | 7.706 | −26.890 | 1.00 | 15.21 |
| ATOM | 802 | CA | PRO | A | 107 | 2.025 | 8.645 | −26.772 | 1.00 | 14.90 |
| ATOM | 803 | CB | PRO | A | 107 | 2.598 | 9.809 | −25.951 | 1.00 | 15.27 |
| ATOM | 804 | CG | PRO | A | 107 | 3.833 | 9.290 | −25.310 | 1.00 | 17.25 |
| ATOM | 805 | CD | PRO | A | 107 | 4.385 | 8.238 | −26.243 | 1.00 | 14.35 |
| ATOM | 806 | C | PRO | A | 107 | 1.468 | 9.219 | −28.066 | 1.00 | 14.48 |
| ATOM | 807 | O | PRO | A | 107 | 0.263 | 9.371 | −28.172 | 1.00 | 13.94 |
| ATOM | 808 | N | LYS | A | 108 | 2.320 | 9.567 | −29.027 | 1.00 | 14.24 |
| ATOM | 809 | CA | LYS | A | 108 | 1.837 | 10.309 | −30.204 | 1.00 | 14.51 |
| ATOM | 810 | CB | LYS | A | 108 | 1.853 | 11.828 | −29.960 | 1.00 | 14.26 |
| ATOM | 811 | CG | LYS | A | 108 | 3.225 | 12.524 | −30.029 | 1.00 | 13.88 |
| ATOM | 812 | CD | LYS | A | 108 | 3.102 | 14.033 | −29.775 | 1.00 | 15.15 |
| ATOM | 813 | CE | LYS | A | 108 | 4.299 | 14.820 | −30.283 | 1.00 | 17.08 |
| ATOM | 814 | NZ | LYS | A | 108 | 4.341 | 16.279 | −29.836 | 1.00 | 15.57 |
| ATOM | 815 | C | LYS | A | 108 | 2.608 | 9.953 | −31.454 | 1.00 | 14.69 |
| ATOM | 816 | O | LYS | A | 108 | 3.734 | 9.442 | −31.377 | 1.00 | 14.67 |
| ATOM | 817 | N | PHE | A | 109 | 1.976 | 10.203 | −32.594 | 1.00 | 14.54 |
| ATOM | 818 | CA | PHE | A | 109 | 2.530 | 9.860 | −33.902 | 1.00 | 15.20 |
| ATOM | 819 | CB | PHE | A | 109 | 1.839 | 8.594 | −34.451 | 1.00 | 15.67 |
| ATOM | 820 | CG | PHE | A | 109 | 1.973 | 7.407 | −33.553 | 1.00 | 16.81 |
| ATOM | 821 | CD1 | PHE | A | 109 | 1.081 | 7.219 | −32.490 | 1.00 | 17.93 |
| ATOM | 822 | CE1 | PHE | A | 109 | 1.205 | 6.117 | −31.649 | 1.00 | 21.93 |
| ATOM | 823 | CZ | PHE | A | 109 | 2.241 | 5.210 | −31.834 | 1.00 | 19.06 |
| ATOM | 824 | CE2 | PHE | A | 109 | 3.141 | 5.384 | −32.883 | 1.00 | 20.27 |
| ATOM | 825 | CD2 | PHE | A | 109 | 3.003 | 6.492 | −33.737 | 1.00 | 19.44 |
| ATOM | 826 | C | PHE | A | 109 | 2.301 | 10.992 | −34.881 | 1.00 | 15.19 |
| ATOM | 827 | O | PHE | A | 109 | 1.450 | 11.861 | −34.655 | 1.00 | 14.41 |
| ATOM | 828 | N | GLU | A | 110 | 3.039 | 10.971 | −35.993 | 1.00 | 15.14 |
| ATOM | 829 | CA | GLU | A | 110 | 2.756 | 11.905 | −37.077 | 1.00 | 15.24 |
| ATOM | 830 | CB | GLU | A | 110 | 3.905 | 11.933 | −38.103 | 1.00 | 15.00 |
| ATOM | 831 | CG | GLU | A | 110 | 5.302 | 12.204 | −37.493 | 1.00 | 16.29 |
| ATOM | 832 | CD | GLU | A | 110 | 5.554 | 13.673 | −37.174 | 1.00 | 17.52 |
| ATOM | 833 | OE1 | GLU | A | 110 | 4.708 | 14.544 | −37.504 | 1.00 | 17.65 |
| ATOM | 834 | OE2 | GLU | A | 110 | 6.619 | 13.963 | −36.587 | 1.00 | 18.74 |
| ATOM | 835 | C | GLU | A | 110 | 1.462 | 11.476 | −37.762 | 1.00 | 15.32 |
| ATOM | 836 | O | GLU | A | 110 | 1.093 | 10.278 | −37.753 | 1.00 | 15.26 |
| ATOM | 837 | N | LEU | A | 111 | 0.776 | 12.445 | −38.360 | 1.00 | 15.59 |
| ATOM | 838 | CA | LEU | A | 111 | −0.522 | 12.186 | −39.009 | 1.00 | 16.33 |
| ATOM | 839 | CB | LEU | A | 111 | −1.265 | 13.510 | −39.163 | 1.00 | 16.57 |
| ATOM | 840 | CG | LEU | A | 111 | −1.770 | 13.908 | −37.756 | 1.00 | 18.11 |
| ATOM | 841 | CD1 | LEU | A | 111 | −1.819 | 15.405 | −37.574 | 1.00 | 21.43 |
| ATOM | 842 | CD2 | LEU | A | 111 | −3.151 | 13.204 | −37.506 | 1.00 | 18.74 |
| ATOM | 843 | C | LEU | A | 111 | −0.409 | 11.436 | −40.350 | 1.00 | 17.37 |
| ATOM | 844 | O | LEU | A | 111 | −1.426 | 11.023 | −40.944 | 1.00 | 17.23 |
| ATOM | 845 | N | THR | A | 112 | 0.833 | 11.258 | −40.815 | 1.00 | 17.42 |
| ATOM | 846 | CA | THR | A | 112 | 1.144 | 10.301 | −41.887 | 1.00 | 17.92 |
| ATOM | 847 | CB | THR | A | 112 | 2.512 | 10.619 | −42.499 | 1.00 | 17.98 |
| ATOM | 848 | OG1 | THR | A | 112 | 3.476 | 10.702 | −41.445 | 1.00 | 18.32 |
| ATOM | 849 | CG2 | THR | A | 112 | 2.486 | 11.945 | −43.228 | 1.00 | 19.47 |
| ATOM | 850 | C | THR | A | 112 | 1.215 | 8.846 | −41.356 | 1.00 | 18.90 |
| ATOM | 851 | O | THR | A | 112 | 1.535 | 7.917 | −42.117 | 1.00 | 17.68 |
| ATOM | 852 | N | LEU | A | 113 | 0.944 | 8.664 | −40.055 | 1.00 | 18.48 |
| ATOM | 853 | CA | LEU | A | 113 | 1.041 | 7.379 | −39.348 | 1.00 | 19.78 |
| ATOM | 854 | CB | LEU | A | 113 | 0.061 | 6.319 | −39.904 | 1.00 | 19.45 |
| ATOM | 855 | CG | LEU | A | 113 | −1.411 | 6.699 | −40.074 | 1.00 | 21.71 |
| ATOM | 856 | CD1 | LEU | A | 113 | −2.194 | 5.470 | −40.477 | 1.00 | 23.46 |
| ATOM | 857 | CD2 | LEU | A | 113 | −2.005 | 7.323 | −38.800 | 1.00 | 21.59 |
| ATOM | 858 | C | LEU | A | 113 | 2.481 | 6.866 | −39.338 | 1.00 | 20.43 |
| ATOM | 859 | O | LEU | A | 113 | 2.737 | 5.704 | −39.653 | 1.00 | 20.96 |
| ATOM | 860 | N | LYS | A | 114 | 3.406 | 7.769 | −39.024 | 1.00 | 20.07 |
| ATOM | 861 | CA | LYS | A | 114 | 4.826 | 7.460 | −38.863 | 1.00 | 20.25 |
| ATOM | 862 | CB | LYS | A | 114 | 5.662 | 8.209 | −39.899 | 1.00 | 20.95 |
| ATOM | 863 | CG | LYS | A | 114 | 5.432 | 7.725 | −41.314 | 1.00 | 25.23 |
| ATOM | 864 | CD | LYS | A | 114 | 6.636 | 8.059 | −42.184 | 1.00 | 33.76 |
| ATOM | 865 | CE | LYS | A | 114 | 6.551 | 7.360 | −43.537 | 1.00 | 38.31 |
| ATOM | 866 | NZ | LYS | A | 114 | 5.285 | 7.711 | −44.251 | 1.00 | 41.29 |
| ATOM | 867 | C | LYS | A | 114 | 5.252 | 7.874 | −37.471 | 1.00 | 19.64 |
| ATOM | 868 | O | LYS | A | 114 | 4.576 | 8.708 | −36.845 | 1.00 | 19.39 |
| ATOM | 869 | N | PRO | A | 115 | 6.376 | 7.318 | −36.973 | 1.00 | 19.06 |
| ATOM | 870 | CA | PRO | A | 115 | 6.750 | 7.626 | −35.601 | 1.00 | 18.72 |
| ATOM | 871 | CB | PRO | A | 115 | 7.963 | 6.712 | −35.326 | 1.00 | 19.82 |
| ATOM | 872 | CG | PRO | A | 115 | 8.101 | 5.814 | −36.500 | 1.00 | 20.93 |
| ATOM | 873 | CD | PRO | A | 115 | 7.339 | 6.412 | −37.639 | 1.00 | 19.08 |
| ATOM | 874 | C | PRO | A | 115 | 7.156 | 9.093 | −35.434 | 1.00 | 18.70 |
| ATOM | 875 | O | PRO | A | 115 | 7.694 | 9.724 | −36.375 | 1.00 | 17.25 |
| ATOM | 876 | N | PHE | A | 116 | 6.844 | 9.628 | −34.256 | 1.00 | 18.55 |
| ATOM | 877 | CA | PHE | A | 116 | 7.342 | 10.918 | −33.805 | 1.00 | 18.27 |
| ATOM | 878 | CB | PHE | A | 116 | 6.359 | 11.566 | −32.809 | 1.00 | 18.41 |
| ATOM | 879 | CG | PHE | A | 116 | 6.908 | 12.804 | −32.151 | 1.00 | 17.33 |
| ATOM | 880 | CD1 | PHE | A | 116 | 6.942 | 14.014 | −32.847 | 1.00 | 16.98 |
| ATOM | 881 | CE1 | PHE | A | 116 | 7.457 | 15.173 | −32.254 | 1.00 | 16.22 |
| ATOM | 882 | CZ | PHE | A | 116 | 7.950 | 15.138 | −30.935 | 1.00 | 15.88 |
| ATOM | 883 | CE2 | PHE | A | 116 | 7.902 | 13.917 | −30.216 | 1.00 | 16.74 |
| ATOM | 884 | CD2 | PHE | A | 116 | 7.380 | 12.767 | −30.825 | 1.00 | 16.05 |
| ATOM | 885 | C | PHE | A | 116 | 8.701 | 10.695 | −33.141 | 1.00 | 19.14 |
| ATOM | 886 | O | PHE | A | 116 | 8.808 | 9.987 | −32.134 | 1.00 | 19.66 |
| ATOM | 887 | N | THR | A | 117 | 9.746 | 11.299 | −33.713 | 1.00 | 19.53 |
| ATOM | 888 | CA | THR | A | 117 | 11.116 | 11.020 | −33.315 | 1.00 | 20.15 |
| ATOM | 889 | CB | THR | A | 117 | 12.042 | 10.999 | −34.567 | 1.00 | 20.83 |
| ATOM | 890 | OG1 | THR | A | 117 | 11.988 | 12.277 | −35.222 | 1.00 | 22.59 |
| ATOM | 891 | CG2 | THR | A | 117 | 11.576 | 9.895 | −35.557 | 1.00 | 21.40 |
| ATOM | 892 | C | THR | A | 117 | 11.685 | 11.969 | −32.263 | 1.00 | 20.34 |
| ATOM | 893 | O | THR | A | 117 | 12.813 | 11.768 | −31.774 | 1.00 | 21.13 |
| ATOM | 894 | N | GLY | A | 118 | 10.943 | 13.017 | −31.914 | 1.00 | 19.25 |
| ATOM | 895 | CA | GLY | A | 118 | 11.451 | 14.018 | −30.974 | 1.00 | 19.41 |
| ATOM | 896 | C | GLY | A | 118 | 11.431 | 13.498 | −29.541 | 1.00 | 19.75 |
| ATOM | 897 | O | GLY | A | 118 | 10.913 | 12.397 | −29.281 | 1.00 | 19.99 |
| ATOM | 898 | N | ASN | A | 119 | 11.998 | 14.279 | −28.622 | 1.00 | 19.56 |
| ATOM | 899 | CA | ASN | A | 119 | 11.958 | 13.954 | −27.198 | 1.00 | 20.41 |
| ATOM | 900 | CB | ASN | A | 119 | 12.961 | 14.801 | −26.419 | 1.00 | 21.50 |
| ATOM | 901 | CG | ASN | A | 119 | 14.377 | 14.612 | −26.930 | 1.00 | 25.14 |
| ATOM | 902 | OD1 | ASN | A | 119 | 14.779 | 13.500 | −27.294 | 1.00 | 30.77 |
| ATOM | 903 | ND2 | ASN | A | 119 | 15.131 | 15.693 | −26.987 | 1.00 | 31.23 |
| ATOM | 904 | C | ASN | A | 119 | 10.550 | 14.194 | −26.696 | 1.00 | 20.06 |
| ATOM | 905 | O | ASN | A | 119 | 9.881 | 15.089 | −27.167 | 1.00 | 19.00 |
| ATOM | 906 | N | TRP | A | 120 | 10.084 | 13.348 | −25.787 | 1.00 | 19.74 |
| ATOM | 907 | CA | TRP | A | 120 | 8.707 | 13.466 | −25.316 | 1.00 | 19.03 |
| ATOM | 908 | CB | TRP | A | 120 | 7.717 | 12.917 | −26.359 | 1.00 | 18.71 |
| ATOM | 909 | CG | TRP | A | 120 | 6.351 | 13.522 | −26.162 | 1.00 | 19.76 |
| ATOM | 910 | CD1 | TRP | A | 120 | 5.239 | 12.901 | −25.673 | 1.00 | 19.81 |
| ATOM | 911 | NE1 | TRP | A | 120 | 4.186 | 13.799 | −25.593 | 1.00 | 19.49 |
| ATOM | 912 | CE2 | TRP | A | 120 | 4.612 | 15.021 | −26.042 | 1.00 | 19.44 |
| ATOM | 913 | CD2 | TRP | A | 120 | 5.975 | 14.886 | −26.410 | 1.00 | 19.35 |
| ATOM | 914 | CE3 | TRP | A | 120 | 6.657 | 16.014 | −26.895 | 1.00 | 18.94 |
| ATOM | 915 | CZ3 | TRP | A | 120 | 5.959 | 17.220 | −27.010 | 1.00 | 20.22 |
| ATOM | 916 | CH2 | TRP | A | 120 | 4.602 | 17.315 | −26.628 | 1.00 | 20.15 |
| ATOM | 917 | CZ2 | TRP | A | 120 | 3.918 | 16.233 | −26.160 | 1.00 | 18.83 |
| ATOM | 918 | C | TRP | A | 120 | 8.602 | 12.685 | −24.001 | 1.00 | 18.80 |
| ATOM | 919 | O | TRP | A | 120 | 9.454 | 11.833 | −23.722 | 1.00 | 18.83 |
| ATOM | 920 | N | GLY | A | 121 | 7.593 | 12.990 | −23.189 | 1.00 | 17.91 |
| ATOM | 921 | CA | GLY | A | 121 | 7.314 | 12.189 | −21.988 | 1.00 | 17.64 |
| ATOM | 922 | C | GLY | A | 121 | 6.721 | 10.834 | −22.362 | 1.00 | 18.93 |
| ATOM | 923 | O | GLY | A | 121 | 5.499 | 10.704 | −22.487 | 1.00 | 18.99 |
| ATOM | 924 | N | ARG | A | 122 | 7.589 | 9.828 | −22.536 | 1.00 | 17.95 |
| ATOM | 925 | CA | ARG | A | 122 | 7.195 | 8.483 | −22.958 | 1.00 | 17.86 |
| ATOM | 926 | CB | ARG | A | 122 | 7.686 | 8.193 | −24.394 | 1.00 | 17.37 |
| ATOM | 927 | CG | ARG | A | 122 | 9.181 | 8.529 | −24.626 | 1.00 | 19.53 |
| ATOM | 928 | CD | ARG | A | 122 | 9.689 | 7.987 | −25.969 | 1.00 | 17.88 |
| ATOM | 929 | NE | ARG | A | 122 | 9.012 | 8.549 | −27.159 | 1.00 | 18.08 |
| ATOM | 930 | CZ | ARG | A | 122 | 9.425 | 9.645 | −27.807 | 1.00 | 18.06 |
| ATOM | 931 | NH1 | ARG | A | 122 | 10.477 | 10.326 | −27.366 | 1.00 | 16.88 |
| ATOM | 932 | NH2 | ARG | A | 122 | 8.784 | 10.074 | −28.892 | 1.00 | 17.35 |
| ATOM | 933 | C | ARG | A | 122 | 7.799 | 7.450 | −21.976 | 1.00 | 17.48 |
| ATOM | 934 | O | ARG | A | 122 | 8.848 | 7.697 | −21.396 | 1.00 | 17.69 |
| ATOM | 935 | N | PRO | A | 123 | 7.142 | 6.298 | −21.781 | 1.00 | 17.14 |
| ATOM | 936 | CA | PRO | A | 123 | 5.886 | 5.916 | −22.382 | 1.00 | 16.01 |
| ATOM | 937 | CB | PRO | A | 123 | 5.908 | 4.385 | −22.266 | 1.00 | 16.51 |
| ATOM | 938 | CG | PRO | A | 123 | 6.585 | 4.144 | −20.969 | 1.00 | 16.30 |
| ATOM | 939 | CD | PRO | A | 123 | 7.658 | 5.238 | −20.873 | 1.00 | 16.95 |
| ATOM | 940 | C | PRO | A | 123 | 4.716 | 6.494 | −21.581 | 1.00 | 15.29 |
| ATOM | 941 | O | PRO | A | 123 | 4.926 | 7.057 | −20.521 | 1.00 | 15.07 |
| ATOM | 942 | N | GLN | A | 124 | 3.504 | 6.362 | −22.120 | 1.00 | 14.60 |
| ATOM | 943 | CA | GLN | A | 124 | 2.289 | 6.675 | −21.386 | 1.00 | 14.40 |
| ATOM | 944 | CB | GLN | A | 124 | 1.602 | 7.889 | −22.001 | 1.00 | 14.71 |
| ATOM | 945 | CG | GLN | A | 124 | 2.442 | 9.186 | −21.711 | 1.00 | 11.93 |
| ATOM | 946 | CD | GLN | A | 124 | 1.993 | 10.407 | −22.472 | 1.00 | 15.67 |
| ATOM | 947 | OE1 | GLN | A | 124 | 2.807 | 11.310 | −22.758 | 1.00 | 15.75 |
| ATOM | 948 | NE2 | GLN | A | 124 | 0.718 | 10.450 | −22.822 | 1.00 | 9.85 |
| ATOM | 949 | C | GLN | A | 124 | 1.441 | 5.421 | −21.511 | 1.00 | 14.28 |
| ATOM | 950 | O | GLN | A | 124 | 0.988 | 5.095 | −22.604 | 1.00 | 14.63 |
| ATOM | 951 | N | ARG | A | 125 | 1.241 | 4.731 | −20.390 | 1.00 | 13.50 |
| ATOM | 952 | CA | ARG | A | 125 | 0.700 | 3.382 | −20.398 | 1.00 | 13.96 |
| ATOM | 953 | CB | ARG | A | 125 | 1.331 | 2.567 | −19.256 | 1.00 | 14.28 |
| ATOM | 954 | CG | ARG | A | 125 | 2.864 | 2.703 | −19.249 | 1.00 | 15.16 |
| ATOM | 955 | CD | ARG | A | 125 | 3.503 | 1.577 | −18.439 | 1.00 | 18.07 |
| ATOM | 956 | NE | ARG | A | 125 | 4.924 | 1.827 | −18.132 | 1.00 | 17.46 |
| ATOM | 957 | CZ | ARG | A | 125 | 5.944 | 1.334 | −18.833 | 1.00 | 19.43 |
| ATOM | 958 | NH1 | ARG | A | 125 | 5.728 | 0.601 | −19.925 | 1.00 | 20.02 |
| ATOM | 959 | NH2 | ARG | A | 125 | 7.197 | 1.596 | −18.453 | 1.00 | 18.94 |
| ATOM | 960 | C | ARG | A | 125 | −0.829 | 3.335 | −20.359 | 1.00 | 14.48 |
| ATOM | 961 | O | ARG | A | 125 | −1.424 | 2.262 | −20.343 | 1.00 | 14.40 |
| ATOM | 962 | N | ASP | A | 126 | −1.462 | 4.509 | −20.374 | 1.00 | 14.03 |
| ATOM | 963 | CA | ASP | A | 126 | −2.919 | 4.568 | −20.542 | 1.00 | 13.39 |
| ATOM | 964 | CB | ASP | A | 126 | −3.488 | 5.922 | −20.067 | 1.00 | 13.17 |
| ATOM | 965 | CG | ASP | A | 126 | −2.926 | 7.092 | −20.845 | 1.00 | 14.17 |
| ATOM | 966 | OD1 | ASP | A | 126 | −1.713 | 7.108 | −21.143 | 1.00 | 12.29 |
| ATOM | 967 | OD2 | ASP | A | 126 | −3.705 | 8.003 | −21.187 | 1.00 | 16.69 |
| ATOM | 968 | C | ASP | A | 126 | −3.350 | 4.306 | −21.974 | 1.00 | 13.60 |
| ATOM | 969 | O | ASP | A | 126 | −4.452 | 3.806 | −22.189 | 1.00 | 13.56 |
| ATOM | 970 | N | GLY | A | 127 | −2.491 | 4.634 | −22.948 | 1.00 | 12.78 |
| ATOM | 971 | CA | GLY | A | 127 | −2.886 | 4.558 | −24.358 | 1.00 | 13.20 |
| ATOM | 972 | C | GLY | A | 127 | −3.473 | 3.213 | −24.794 | 1.00 | 12.77 |
| ATOM | 973 | O | GLY | A | 127 | −4.579 | 3.150 | −25.327 | 1.00 | 12.56 |
| ATOM | 974 | N | PRO | A | 128 | −2.720 | 2.120 | −24.613 | 1.00 | 13.94 |
| ATOM | 975 | CA | PRO | A | 128 | −3.262 | 0.810 | −24.978 | 1.00 | 12.97 |
| ATOM | 976 | CB | PRO | A | 128 | −2.135 | −0.162 | −24.552 | 1.00 | 14.15 |
| ATOM | 977 | CG | PRO | A | 128 | −0.907 | 0.656 | −24.721 | 1.00 | 14.39 |
| ATOM | 978 | CD | PRO | A | 128 | −1.323 | 2.010 | −24.150 | 1.00 | 13.67 |
| ATOM | 979 | C | PRO | A | 128 | −4.571 | 0.455 | −24.255 | 1.00 | 13.30 |
| ATOM | 980 | O | PRO | A | 128 | −5.433 | −0.161 | −24.869 | 1.00 | 13.72 |
| ATOM | 981 | N | ALA | A | 129 | −4.718 | 0.852 | −22.985 | 1.00 | 12.95 |
| ATOM | 982 | CA | ALA | A | 129 | −5.963 | 0.611 | −22.258 | 1.00 | 13.49 |
| ATOM | 983 | CB | ALA | A | 129 | −5.806 | 1.016 | −20.808 | 1.00 | 13.00 |
| ATOM | 984 | C | ALA | A | 129 | −7.162 | 1.329 | −22.923 | 1.00 | 13.43 |
| ATOM | 985 | O | ALA | A | 129 | −8.217 | 0.721 | −23.159 | 1.00 | 13.26 |
| ATOM | 986 | N | LEU | A | 130 | −6.998 | 2.619 | −23.221 | 1.00 | 12.51 |
| ATOM | 987 | CA | LEU | A | 130 | −8.068 | 3.409 | −23.813 | 1.00 | 12.69 |
| ATOM | 988 | CB | LEU | A | 130 | −7.678 | 4.903 | −23.806 | 1.00 | 12.56 |
| ATOM | 989 | CG | LEU | A | 130 | −7.458 | 5.555 | −22.426 | 1.00 | 14.76 |
| ATOM | 990 | CD1 | LEU | A | 130 | −6.959 | 6.991 | −22.643 | 1.00 | 15.23 |
| ATOM | 991 | CD2 | LEU | A | 130 | −8.776 | 5.544 | −21.651 | 1.00 | 15.30 |
| ATOM | 992 | C | LEU | A | 130 | −8.410 | 2.934 | −25.228 | 1.00 | 12.78 |
| ATOM | 993 | O | LEU | A | 130 | −9.571 | 2.863 | −25.607 | 1.00 | 12.83 |
| ATOM | 994 | N | ARG | A | 131 | −7.386 | 2.601 | −26.015 | 1.00 | 13.70 |
| ATOM | 995 | CA | ARG | A | 131 | −7.630 | 2.070 | −27.351 | 1.00 | 14.52 |
| ATOM | 996 | CB | ARG | A | 131 | −6.316 | 1.925 | −28.135 | 1.00 | 14.04 |
| ATOM | 997 | CG | ARG | A | 131 | −6.550 | 1.438 | −29.566 | 1.00 | 15.60 |
| ATOM | 998 | CD | ARG | A | 131 | −5.278 | 1.522 | −30.428 | 1.00 | 15.59 |
| ATOM | 999 | NE | ARG | A | 131 | −4.118 | 0.915 | −29.779 | 1.00 | 16.77 |
| ATOM | 1000 | CZ | ARG | A | 131 | −2.860 | 1.205 | −30.098 | 1.00 | 16.26 |
| ATOM | 1001 | NH1 | ARG | A | 131 | −2.610 | 2.104 | −31.054 | 1.00 | 16.37 |
| ATOM | 1002 | NH2 | ARG | A | 131 | −1.856 | 0.618 | −29.448 | 1.00 | 15.96 |
| ATOM | 1003 | C | ARG | A | 131 | −8.408 | 0.729 | −27.283 | 1.00 | 14.44 |
| ATOM | 1004 | O | ARG | A | 131 | −9.350 | 0.533 | −28.050 | 1.00 | 15.71 |
| ATOM | 1005 | N | ALA | A | 132 | −8.025 | −0.164 | −26.364 | 1.00 | 14.50 |
| ATOM | 1006 | CA | ALA | A | 132 | −8.738 | −1.456 | −26.195 | 1.00 | 15.06 |
| ATOM | 1007 | CB | ALA | A | 132 | −8.069 | −2.348 | −25.112 | 1.00 | 15.02 |
| ATOM | 1008 | C | ALA | A | 132 | −10.194 | −1.197 | −25.846 | 1.00 | 15.43 |
| ATOM | 1009 | O | ALA | A | 132 | −11.101 | −1.808 | −26.416 | 1.00 | 15.57 |
| ATOM | 1010 | N | ILE | A | 133 | −10.418 | −0.270 | −24.915 | 1.00 | 15.70 |
| ATOM | 1011 | CA | ILE | A | 133 | −11.777 | 0.049 | −24.491 | 1.00 | 14.44 |
| ATOM | 1012 | CB | ILE | A | 133 | −11.775 | 1.056 | −23.335 | 1.00 | 14.07 |
| ATOM | 1013 | CG1 | ILE | A | 133 | −11.268 | 0.387 | −22.046 | 1.00 | 15.40 |
| ATOM | 1014 | CD1 | ILE | A | 133 | −10.751 | 1.388 | −21.017 | 1.00 | 16.66 |
| ATOM | 1015 | CG2 | ILE | A | 133 | −13.176 | 1.702 | −23.101 | 1.00 | 13.98 |
| ATOM | 1016 | C | ILE | A | 133 | −12.633 | 0.517 | −25.679 | 1.00 | 14.36 |
| ATOM | 1017 | O | ILE | A | 133 | −13.781 | 0.102 | −25.807 | 1.00 | 14.69 |
| ATOM | 1018 | N | ALA | A | 134 | −12.079 | 1.362 | −26.545 | 1.00 | 13.69 |
| ATOM | 1019 | CA | ALA | A | 134 | −12.819 | 1.832 | −27.720 | 1.00 | 13.81 |
| ATOM | 1020 | CB | ALA | A | 134 | −12.019 | 2.949 | −28.452 | 1.00 | 13.99 |
| ATOM | 1021 | C | ALA | A | 134 | −13.140 | 0.662 | −28.657 | 1.00 | 14.62 |
| ATOM | 1022 | O | ALA | A | 134 | −14.279 | 0.473 | −29.087 | 1.00 | 14.90 |
| ATOM | 1023 | N | LEU | A | 135 | −12.133 | −0.155 | −28.947 | 1.00 | 14.66 |
| ATOM | 1024 | CA | LEU | A | 135 | −12.328 | −1.251 | −29.901 | 1.00 | 15.75 |
| ATOM | 1025 | CB | LEU | A | 135 | −10.984 | −1.875 | −30.311 | 1.00 | 15.75 |
| ATOM | 1026 | CG | LEU | A | 135 | −10.348 | −1.231 | −31.557 | 1.00 | 16.40 |
| ATOM | 1027 | CD1 | LEU | A | 135 | −10.257 | 0.308 | −31.471 | 1.00 | 18.56 |
| ATOM | 1028 | CD2 | LEU | A | 135 | −8.980 | −1.852 | −31.804 | 1.00 | 17.85 |
| ATOM | 1029 | C | LEU | A | 135 | −13.277 | −2.306 | −29.340 | 1.00 | 15.13 |
| ATOM | 1030 | O | LEU | A | 135 | −14.079 | −2.845 | −30.087 | 1.00 | 15.84 |
| ATOM | 1031 | N | ILE | A | 136 | −13.192 | −2.573 | −28.039 | 1.00 | 15.22 |
| ATOM | 1032 | CA | ILE | A | 136 | −14.153 | −3.473 | −27.377 | 1.00 | 15.71 |
| ATOM | 1033 | CB | ILE | A | 136 | −13.734 | −3.829 | −25.918 | 1.00 | 15.92 |
| ATOM | 1034 | CG1 | ILE | A | 136 | −12.408 | −4.598 | −25.904 | 1.00 | 15.39 |
| ATOM | 1035 | CD1 | ILE | A | 136 | −11.742 | −4.679 | −24.497 | 1.00 | 15.63 |
| ATOM | 1036 | CG2 | ILE | A | 136 | −14.842 | −4.611 | −25.204 | 1.00 | 16.20 |
| ATOM | 1037 | C | ILE | A | 136 | −15.565 | −2.902 | −27.457 | 1.00 | 17.10 |
| ATOM | 1038 | O | ILE | A | 136 | −16.531 | −3.631 | −27.728 | 1.00 | 17.48 |
| ATOM | 1039 | N | GLY | A | 137 | −15.685 | −1.581 | −27.297 | 1.00 | 16.46 |
| ATOM | 1040 | CA | GLY | A | 137 | −16.979 | −0.902 | −27.484 | 1.00 | 16.59 |
| ATOM | 1041 | C | GLY | A | 137 | −17.600 | −1.206 | −28.833 | 1.00 | 17.13 |
| ATOM | 1042 | O | GLY | A | 137 | −18.778 | −1.605 | −28.920 | 1.00 | 16.92 |
| ATOM | 1043 | N | TYR | A | 138 | −16.817 | −1.056 | −29.898 | 1.00 | 16.61 |
| ATOM | 1044 | CA | TYR | A | 138 | −17.353 | −1.349 | −31.224 | 1.00 | 17.93 |
| ATOM | 1045 | CB | TYR | A | 138 | −16.446 | −0.838 | −32.341 | 1.00 | 17.46 |
| ATOM | 1046 | CG | TYR | A | 138 | −17.112 | −0.897 | −33.693 | 1.00 | 18.28 |
| ATOM | 1047 | CD1 | TYR | A | 138 | −18.350 | −0.268 | −33.914 | 1.00 | 18.85 |
| ATOM | 1048 | CE1 | TYR | A | 138 | −18.966 | −0.314 | −35.153 | 1.00 | 21.29 |
| ATOM | 1049 | CZ | TYR | A | 138 | −18.358 | −1.001 | −36.207 | 1.00 | 20.99 |
| ATOM | 1050 | OH | TYR | A | 138 | −18.994 | −1.055 | −37.433 | 1.00 | 20.06 |
| ATOM | 1051 | CE2 | TYR | A | 138 | −17.133 | −1.636 | −36.026 | 1.00 | 19.88 |
| ATOM | 1052 | CD2 | TYR | A | 138 | −16.512 | −1.583 | −34.766 | 1.00 | 18.25 |
| ATOM | 1053 | C | TYR | A | 138 | −17.643 | −2.844 | −31.406 | 1.00 | 18.67 |
| ATOM | 1054 | O | TYR | A | 138 | −18.654 | −3.207 | −32.037 | 1.00 | 20.06 |
| ATOM | 1055 | N | SER | A | 139 | −16.766 | −3.686 | −30.864 | 1.00 | 19.48 |
| ATOM | 1056 | CA | SER | A | 139 | −16.942 | −5.146 | −30.900 | 1.00 | 21.02 |
| ATOM | 1057 | CB | SER | A | 139 | −15.808 | −5.829 | −30.129 | 1.00 | 21.09 |
| ATOM | 1058 | OG | SER | A | 139 | −14.581 | −5.598 | −30.789 | 1.00 | 21.49 |
| ATOM | 1059 | C | SER | A | 139 | −18.298 | −5.557 | −30.325 | 1.00 | 22.24 |
| ATOM | 1060 | O | SER | A | 139 | −19.002 | −6.392 | −30.907 | 1.00 | 23.64 |
| ATOM | 1061 | N | LYS | A | 140 | −18.669 | −4.976 | −29.188 | 1.00 | 22.77 |
| ATOM | 1062 | CA | LYS | A | 140 | −19.987 | −5.225 | −28.595 | 1.00 | 24.29 |
| ATOM | 1063 | CB | LYS | A | 140 | −20.218 | −4.343 | −27.370 | 1.00 | 24.29 |
| ATOM | 1064 | CG | LYS | A | 140 | −19.384 | −4.695 | −26.170 | 1.00 | 26.62 |
| ATOM | 1065 | CD | LYS | A | 140 | −19.696 | −3.693 | −25.060 | 1.00 | 28.24 |
| ATOM | 1066 | CE | LYS | A | 140 | −18.589 | −3.635 | −24.056 | 1.00 | 28.19 |
| ATOM | 1067 | NZ | LYS | A | 140 | −18.940 | −2.725 | −22.954 | 1.00 | 26.78 |
| ATOM | 1068 | C | LYS | A | 140 | −21.126 | −5.001 | −29.584 | 1.00 | 24.17 |
| ATOM | 1069 | O | LYS | A | 140 | −22.053 | −5.823 | −29.670 | 1.00 | 24.94 |
| ATOM | 1070 | N | TRP | A | 141 | −21.062 | −3.898 | −30.321 | 1.00 | 23.60 |
| ATOM | 1071 | CA | TRP | A | 141 | −22.054 | −3.613 | −31.338 | 1.00 | 24.29 |
| ATOM | 1072 | CB | TRP | A | 141 | −21.847 | −2.226 | −31.953 | 1.00 | 24.36 |
| ATOM | 1073 | CG | TRP | A | 141 | −22.973 | −1.833 | −32.874 | 1.00 | 24.25 |
| ATOM | 1074 | CD1 | TRP | A | 141 | −24.113 | −1.170 | −32.531 | 1.00 | 25.06 |
| ATOM | 1075 | NE1 | TRP | A | 141 | −24.921 | −1.016 | −33.638 | 1.00 | 25.22 |
| ATOM | 1076 | CE2 | TRP | A | 141 | −24.302 | −1.575 | −34.722 | 1.00 | 24.07 |
| ATOM | 1077 | CD2 | TRP | A | 141 | −23.078 | −2.115 | −34.276 | 1.00 | 24.80 |
| ATOM | 1078 | CE3 | TRP | A | 141 | −22.248 | −2.766 | −35.203 | 1.00 | 25.40 |
| ATOM | 1079 | CZ3 | TRP | A | 141 | −22.669 | −2.858 | −36.532 | 1.00 | 25.72 |
| ATOM | 1080 | CH2 | TRP | A | 141 | −23.891 | −2.304 | −36.940 | 1.00 | 24.95 |
| ATOM | 1081 | CZ2 | TRP | A | 141 | −24.721 | −1.666 | −36.051 | 1.00 | 25.14 |
| ATOM | 1082 | C | TRP | A | 141 | −22.078 | −4.666 | −32.448 | 1.00 | 24.47 |
| ATOM | 1083 | O | TRP | A | 141 | −23.155 | −5.152 | −32.831 | 1.00 | 24.52 |
| ATOM | 1084 | N | LEU | A | 142 | −20.904 | −4.991 | −32.985 | 1.00 | 24.39 |
| ATOM | 1085 | CA | LEU | A | 142 | −20.806 | −6.024 | −34.010 | 1.00 | 25.05 |
| ATOM | 1086 | CB | LEU | A | 142 | −19.361 | −6.199 | −34.473 | 1.00 | 24.56 |
| ATOM | 1087 | CG | LEU | A | 142 | −18.754 | −5.023 | −35.252 | 1.00 | 24.44 |
| ATOM | 1088 | CD1 | LEU | A | 142 | −17.274 | −5.304 | −35.441 | 1.00 | 23.72 |
| ATOM | 1089 | CD2 | LEU | A | 142 | −19.424 | −4.793 | −36.624 | 1.00 | 25.76 |
| ATOM | 1090 | C | LEU | A | 142 | −21.406 | −7.364 | −33.556 | 1.00 | 25.97 |
| ATOM | 1091 | O | LEU | A | 142 | −22.195 | −7.966 | −34.283 | 1.00 | 26.49 |
| ATOM | 1092 | N | ILE | A | 143 | −21.045 | −7.814 | −32.359 | 1.00 | 27.04 |
| ATOM | 1093 | CA | ILE | A | 143 | −21.596 | −9.040 | −31.792 | 1.00 | 28.46 |
| ATOM | 1094 | CB | ILE | A | 143 | −20.959 | −9.362 | −30.425 | 1.00 | 28.30 |
| ATOM | 1095 | CG1 | ILE | A | 143 | −19.474 | −9.722 | −30.609 | 1.00 | 27.97 |
| ATOM | 1096 | CD1 | ILE | A | 143 | −18.707 | −9.814 | −29.301 | 1.00 | 29.85 |
| ATOM | 1097 | CG2 | ILE | A | 143 | −21.720 | −10.494 | −29.717 | 1.00 | 29.57 |
| ATOM | 1098 | C | ILE | A | 143 | −23.124 | −8.992 | −31.682 | 1.00 | 29.52 |
| ATOM | 1099 | O | ILE | A | 143 | −23.813 | −9.928 | −32.118 | 1.00 | 30.10 |
| ATOM | 1100 | N | ASN | A | 144 | −23.655 | −7.916 | −31.111 | 1.00 | 30.37 |
| ATOM | 1101 | CA | ASN | A | 144 | −25.109 | −7.768 | −30.988 | 1.00 | 32.18 |
| ATOM | 1102 | CB | ASN | A | 144 | −25.479 | −6.522 | −30.186 | 1.00 | 32.73 |
| ATOM | 1103 | CG | ASN | A | 144 | −26.960 | −6.489 | −29.792 | 1.00 | 36.88 |
| ATOM | 1104 | OD1 | ASN | A | 144 | −27.444 | −7.350 | −29.041 | 1.00 | 42.25 |
| ATOM | 1105 | ND2 | ASN | A | 144 | −27.685 | −5.488 | −30.291 | 1.00 | 40.10 |
| ATOM | 1106 | C | ASN | A | 144 | −25.820 | −7.760 | −32.341 | 1.00 | 32.17 |
| ATOM | 1107 | O | ASN | A | 144 | −27.012 | −8.029 | −32.411 | 1.00 | 32.73 |
| ATOM | 1108 | N | ASN | A | 145 | −25.094 | −7.460 | −33.411 | 1.00 | 32.19 |
| ATOM | 1109 | CA | ASN | A | 145 | −25.705 | −7.403 | −34.726 | 1.00 | 32.78 |
| ATOM | 1110 | CB | ASN | A | 145 | −25.526 | −6.014 | −35.331 | 1.00 | 33.16 |
| ATOM | 1111 | CG | ASN | A | 145 | −26.397 | −4.986 | −34.639 | 1.00 | 34.32 |
| ATOM | 1112 | OD1 | ASN | A | 145 | −27.576 | −4.841 | −34.969 | 1.00 | 37.42 |
| ATOM | 1113 | ND2 | ASN | A | 145 | −25.834 | −4.289 | −33.647 | 1.00 | 34.31 |
| ATOM | 1114 | C | ASN | A | 145 | −25.285 | −8.533 | −35.671 | 1.00 | 33.13 |
| ATOM | 1115 | O | ASN | A | 145 | −25.412 | −8.415 | −36.902 | 1.00 | 33.03 |
| ATOM | 1116 | N | ASN | A | 146 | −24.789 | −9.618 | −35.065 | 1.00 | 33.43 |
| ATOM | 1117 | CA | ASN | A | 146 | −24.475 | −10.885 | −35.736 | 1.00 | 34.14 |
| ATOM | 1118 | CB | ASN | A | 146 | −25.710 | −11.459 | −36.461 | 1.00 | 34.85 |
| ATOM | 1119 | CG | ASN | A | 146 | −26.994 | −11.280 | −35.657 | 1.00 | 37.41 |
| ATOM | 1120 | OD1 | ASN | A | 146 | −27.033 | −11.543 | −34.450 | 1.00 | 41.43 |
| ATOM | 1121 | ND2 | ASN | A | 146 | −28.047 | −10.814 | −36.321 | 1.00 | 41.54 |
| ATOM | 1122 | C | ASN | A | 146 | −23.266 | −10.795 | −36.652 | 1.00 | 33.92 |
| ATOM | 1123 | O | ASN | A | 146 | −23.216 | −11.419 | −37.724 | 1.00 | 33.76 |
| ATOM | 1124 | N | TYR | A | 147 | −22.280 | −10.013 | −36.221 | 1.00 | 32.92 |
| ATOM | 1125 | CA | TYR | A | 147 | −21.049 | −9.870 | −36.974 | 1.00 | 32.90 |
| ATOM | 1126 | CB | TYR | A | 147 | −20.859 | −8.423 | −37.451 | 1.00 | 33.31 |
| ATOM | 1127 | CG | TYR | A | 147 | −21.966 | −7.893 | −38.339 | 1.00 | 33.29 |
| ATOM | 1128 | CD1 | TYR | A | 147 | −22.168 | −8.410 | −39.621 | 1.00 | 34.11 |
| ATOM | 1129 | CE1 | TYR | A | 147 | −23.177 | −7.924 | −40.438 | 1.00 | 34.19 |
| ATOM | 1130 | CZ | TYR | A | 147 | −23.986 | −6.888 | −39.983 | 1.00 | 34.10 |
| ATOM | 1131 | OH | TYR | A | 147 | −24.987 | −6.399 | −40.794 | 1.00 | 35.08 |
| ATOM | 1132 | CE2 | TYR | A | 147 | −23.798 | −6.345 | −38.722 | 1.00 | 32.48 |
| ATOM | 1133 | CD2 | TYR | A | 147 | −22.792 | −6.852 | −37.906 | 1.00 | 32.03 |
| ATOM | 1134 | C | TYR | A | 147 | −19.857 | −10.297 | −36.138 | 1.00 | 32.74 |
| ATOM | 1135 | O | TYR | A | 147 | −18.795 | −9.710 | −36.242 | 1.00 | 32.03 |
| ATOM | 1136 | N | GLN | A | 148 | −20.037 | −11.325 | −35.312 | 1.00 | 33.44 |
| ATOM | 1137 | CA | GLN | A | 148 | −18.977 | −11.807 | −34.427 | 1.00 | 34.39 |
| ATOM | 1138 | CB | GLN | A | 148 | −19.483 | −12.971 | −33.573 | 1.00 | 34.76 |
| ATOM | 1139 | CG | GLN | A | 148 | −18.523 | −13.445 | −32.481 | 1.00 | 35.56 |
| ATOM | 1140 | CD | GLN | A | 148 | −19.216 | −14.273 | −31.401 | 1.00 | 36.59 |
| ATOM | 1141 | OE1 | GLN | A | 148 | −20.296 | −13.913 | −30.916 | 1.00 | 41.20 |
| ATOM | 1142 | NE2 | GLN | A | 148 | −18.589 | −15.380 | −31.008 | 1.00 | 38.65 |
| ATOM | 1143 | C | GLN | A | 148 | −17.690 | −12.196 | −35.176 | 1.00 | 34.46 |
| ATOM | 1144 | O | GLN | A | 148 | −16.582 | −12.002 | −34.654 | 1.00 | 34.20 |
| ATOM | 1145 | N | PHE | A | 149 | −17.841 | −12.735 | −36.391 | 1.00 | 34.19 |
| ATOM | 1146 | CA | PHE | A | 149 | −16.696 | −13.131 | −37.217 | 1.00 | 34.10 |
| ATOM | 1147 | CB | PHE | A | 149 | −17.140 | −13.804 | −38.534 | 1.00 | 35.60 |
| ATOM | 1148 | CG | PHE | A | 149 | −18.346 | −13.168 | −39.193 | 1.00 | 38.70 |
| ATOM | 1149 | CD1 | PHE | A | 149 | −19.388 | −13.976 | −39.676 | 1.00 | 42.72 |
| ATOM | 1150 | CE1 | PHE | A | 149 | −20.518 | −13.417 | −40.295 | 1.00 | 43.69 |
| ATOM | 1151 | CZ | PHE | A | 149 | −20.615 | −12.019 | −40.437 | 1.00 | 43.12 |
| ATOM | 1152 | CE2 | PHE | A | 149 | −19.567 | −11.188 | −39.953 | 1.00 | 43.20 |
| ATOM | 1153 | CD2 | PHE | A | 149 | −18.451 | −11.772 | −39.341 | 1.00 | 42.33 |
| ATOM | 1154 | C | PHE | A | 149 | −15.746 | −11.960 | −37.517 | 1.00 | 32.49 |
| ATOM | 1155 | O | PHE | A | 149 | −14.528 | −12.132 | −37.548 | 1.00 | 32.23 |
| ATOM | 1156 | N | THR | A | 150 | −16.327 | −10.789 | −37.751 | 1.00 | 30.95 |
| ATOM | 1157 | CA | THR | A | 150 | −15.570 | −9.568 | −38.040 | 1.00 | 29.68 |
| ATOM | 1158 | CB | THR | A | 150 | −16.512 | −8.424 | −38.445 | 1.00 | 29.97 |
| ATOM | 1159 | OG1 | THR | A | 150 | −17.162 | −8.768 | −39.673 | 1.00 | 30.58 |
| ATOM | 1160 | CG2 | THR | A | 150 | −15.758 | −7.096 | −38.637 | 1.00 | 29.18 |
| ATOM | 1161 | C | THR | A | 150 | −14.727 | −9.203 | −36.822 | 1.00 | 28.54 |
| ATOM | 1162 | O | THR | A | 150 | −13.566 | −8.827 | −36.965 | 1.00 | 28.38 |
| ATOM | 1163 | N | VAL | A | 151 | −15.310 | −9.354 | −35.636 | 1.00 | 27.23 |
| ATOM | 1164 | CA | VAL | A | 151 | −14.597 | −9.146 | −34.374 | 1.00 | 26.78 |
| ATOM | 1165 | CB | VAL | A | 151 | −15.529 | −9.352 | −33.148 | 1.00 | 26.25 |
| ATOM | 1166 | CG1 | VAL | A | 151 | −14.752 | −9.213 | −31.832 | 1.00 | 26.50 |
| ATOM | 1167 | CG2 | VAL | A | 151 | −16.690 | −8.361 | −33.178 | 1.00 | 24.41 |
| ATOM | 1168 | C | VAL | A | 151 | −13.384 | −10.080 | −34.305 | 1.00 | 27.61 |
| ATOM | 1169 | O | VAL | A | 151 | −12.246 | −9.638 | −34.106 | 1.00 | 26.67 |
| ATOM | 1170 | N | SER | A | 152 | −13.625 | −11.375 | −34.505 | 1.00 | 28.53 |
| ATOM | 1171 | CA | SER | A | 152 | −12.551 | −12.369 | −34.470 | 1.00 | 30.01 |
| ATOM | 1172 | CB | SER | A | 152 | −13.102 | −13.759 | −34.773 | 1.00 | 30.18 |
| ATOM | 1173 | OG | SER | A | 152 | −13.612 | −14.300 | −33.586 | 1.00 | 32.28 |
| ATOM | 1174 | C | SER | A | 152 | −11.419 | −12.091 | −35.430 | 1.00 | 30.23 |
| ATOM | 1175 | O | SER | A | 152 | −10.250 | −12.250 | −35.090 | 1.00 | 30.95 |
| ATOM | 1176 | N | ASN | A | 153 | −11.762 | −11.705 | −36.641 | 1.00 | 31.46 |
| ATOM | 1177 | CA | ASN | A | 153 | −10.753 | −11.624 | −37.674 | 1.00 | 32.41 |
| ATOM | 1178 | CB | ASN | A | 153 | −11.333 | −12.118 | −38.997 | 1.00 | 33.62 |
| ATOM | 1179 | CG | ASN | A | 153 | −11.791 | −13.584 | −38.902 | 1.00 | 36.13 |
| ATOM | 1180 | OD1 | ASN | A | 153 | −12.931 | −13.918 | −39.231 | 1.00 | 40.99 |
| ATOM | 1181 | ND2 | ASN | A | 153 | −10.917 | −14.444 | −38.383 | 1.00 | 37.61 |
| ATOM | 1182 | C | ASN | A | 153 | −10.060 | −10.272 | −37.787 | 1.00 | 32.10 |
| ATOM | 1183 | O | ASN | A | 153 | −8.850 | −10.213 | −38.020 | 1.00 | 32.79 |
| ATOM | 1184 | N | VAL | A | 154 | −10.810 | −9.193 | −37.577 | 1.00 | 30.55 |
| ATOM | 1185 | CA | VAL | A | 154 | −10.251 | −7.854 | −37.750 | 1.00 | 28.93 |
| ATOM | 1186 | CB | VAL | A | 154 | −11.217 | −6.925 | −38.537 | 1.00 | 29.03 |
| ATOM | 1187 | CG1 | VAL | A | 154 | −10.565 | −5.577 | −38.827 | 1.00 | 29.23 |
| ATOM | 1188 | CG2 | VAL | A | 154 | −11.654 | −7.585 | −39.860 | 1.00 | 29.87 |
| ATOM | 1189 | C | VAL | A | 154 | −9.824 | −7.211 | −36.414 | 1.00 | 27.28 |
| ATOM | 1190 | O | VAL | A | 154 | −8.722 | −6.678 | −36.306 | 1.00 | 26.96 |
| ATOM | 1191 | N | ILE | A | 155 | −10.685 | −7.288 | −35.403 | 1.00 | 25.13 |
| ATOM | 1192 | CA | ILE | A | 155 | −10.525 | −6.459 | −34.197 | 1.00 | 23.05 |
| ATOM | 1193 | CB | ILE | A | 155 | −11.900 | −5.972 | −33.670 | 1.00 | 23.10 |
| ATOM | 1194 | CG1 | ILE | A | 155 | −12.596 | −5.128 | −34.741 | 1.00 | 22.49 |
| ATOM | 1195 | CD1 | ILE | A | 155 | −14.006 | −4.680 | −34.375 | 1.00 | 22.72 |
| ATOM | 1196 | CG2 | ILE | A | 155 | −11.731 | −5.144 | −32.399 | 1.00 | 23.05 |
| ATOM | 1197 | C | ILE | A | 155 | −9.710 | −7.136 | −33.092 | 1.00 | 22.44 |
| ATOM | 1198 | O | ILE | A | 155 | −8.789 | −6.537 | −32.533 | 1.00 | 21.14 |
| ATOM | 1199 | N | TRP | A | 156 | −10.006 | −8.409 | −32.822 | 1.00 | 21.48 |
| ATOM | 1200 | CA | TRP | A | 156 | −9.392 | −9.099 | −31.696 | 1.00 | 21.75 |
| ATOM | 1201 | CB | TRP | A | 156 | −9.958 | −10.520 | −31.511 | 1.00 | 22.50 |
| ATOM | 1202 | CG | TRP | A | 156 | −9.298 | −11.245 | −30.371 | 1.00 | 23.43 |
| ATOM | 1203 | CD1 | TRP | A | 156 | −8.420 | −12.298 | −30.461 | 1.00 | 24.92 |
| ATOM | 1204 | NE1 | TRP | A | 156 | −8.011 | −12.680 | −29.198 | 1.00 | 24.85 |
| ATOM | 1205 | CE2 | TRP | A | 156 | −8.600 | −11.863 | −28.269 | 1.00 | 26.38 |
| ATOM | 1206 | CD2 | TRP | A | 156 | −9.416 | −10.941 | −28.970 | 1.00 | 25.03 |
| ATOM | 1207 | CE3 | TRP | A | 156 | −10.139 | −9.983 | −28.236 | 1.00 | 25.01 |
| ATOM | 1208 | CZ3 | TRP | A | 156 | −10.024 | −9.982 | −26.844 | 1.00 | 24.30 |
| ATOM | 1209 | CH2 | TRP | A | 156 | −9.206 | −10.910 | −26.185 | 1.00 | 23.97 |
| ATOM | 1210 | CZ2 | TRP | A | 156 | −8.495 | −11.861 | −26.875 | 1.00 | 24.60 |
| ATOM | 1211 | C | TRP | A | 156 | −7.845 | −9.109 | −31.699 | 1.00 | 21.52 |
| ATOM | 1212 | O | TRP | A | 156 | −7.235 | −8.945 | −30.648 | 1.00 | 21.78 |
| ATOM | 1213 | N | PRO | A | 157 | −7.209 | −9.303 | −32.870 | 1.00 | 21.66 |
| ATOM | 1214 | CA | PRO | A | 157 | −5.726 | −9.258 | −32.878 | 1.00 | 21.40 |
| ATOM | 1215 | CB | PRO | A | 157 | −5.378 | −9.459 | −34.360 | 1.00 | 21.26 |
| ATOM | 1216 | CG | PRO | A | 157 | −6.583 | −10.172 | −34.955 | 1.00 | 22.82 |
| ATOM | 1217 | CD | PRO | A | 157 | −7.762 | −9.596 | −34.207 | 1.00 | 21.45 |
| ATOM | 1218 | C | PRO | A | 157 | −5.162 | −7.898 | −32.410 | 1.00 | 21.30 |
| ATOM | 1219 | O | PRO | A | 157 | −4.092 | −7.837 | −31.795 | 1.00 | 21.11 |
| ATOM | 1220 | N | ILE | A | 158 | −5.881 | −6.821 | −32.724 | 1.00 | 20.52 |
| ATOM | 1221 | CA | ILE | A | 158 | −5.457 | −5.467 | −32.318 | 1.00 | 19.93 |
| ATOM | 1222 | CB | ILE | A | 158 | −6.273 | −4.348 | −33.034 | 1.00 | 19.73 |
| ATOM | 1223 | CG1 | ILE | A | 158 | −6.261 | −4.527 | −34.559 | 1.00 | 21.25 |
| ATOM | 1224 | CD1 | ILE | A | 158 | −7.229 | −3.640 | −35.351 | 1.00 | 20.03 |
| ATOM | 1225 | CG2 | ILE | A | 158 | −5.686 | −2.971 | −32.670 | 1.00 | 20.16 |
| ATOM | 1226 | C | ILE | A | 158 | −5.632 | −5.366 | −30.816 | 1.00 | 19.58 |
| ATOM | 1227 | O | ILE | A | 158 | −4.701 | −5.023 | −30.081 | 1.00 | 19.04 |
| ATOM | 1228 | N | VAL | A | 159 | −6.840 | −5.704 | −30.359 | 1.00 | 19.39 |
| ATOM | 1229 | CA | VAL | A | 159 | −7.201 | −5.624 | −28.953 | 1.00 | 19.15 |
| ATOM | 1230 | CB | VAL | A | 159 | −8.687 | −6.026 | −28.744 | 1.00 | 19.06 |
| ATOM | 1231 | CG1 | VAL | A | 159 | −9.046 | −6.028 | −27.253 | 1.00 | 20.39 |
| ATOM | 1232 | CG2 | VAL | A | 159 | −9.604 | −5.090 | −29.511 | 1.00 | 20.08 |
| ATOM | 1233 | C | VAL | A | 159 | −6.280 | −6.501 | −28.105 | 1.00 | 19.39 |
| ATOM | 1234 | O | VAL | A | 159 | −5.794 | −6.089 | −27.036 | 1.00 | 18.63 |
| ATOM | 1235 | N | ARG | A | 160 | −6.022 | −7.721 | −28.585 | 1.00 | 18.93 |
| ATOM | 1236 | CA | ARG | A | 160 | −5.171 | −8.633 | −27.833 | 1.00 | 19.64 |
| ATOM | 1237 | CB | ARG | A | 160 | −5.078 | −10.005 | −28.513 | 1.00 | 19.17 |
| ATOM | 1238 | CG | ARG | A | 160 | −4.064 | −10.942 | −27.872 | 1.00 | 21.41 |
| ATOM | 1239 | CD | ARG | A | 160 | −3.978 | −12.278 | −28.637 | 1.00 | 23.47 |
| ATOM | 1240 | NE | ARG | A | 160 | −3.542 | −12.066 | −30.021 | 1.00 | 29.25 |
| ATOM | 1241 | CZ | ARG | A | 160 | −3.963 | −12.771 | −31.074 | 1.00 | 33.46 |
| ATOM | 1242 | NH1 | ARG | A | 160 | −4.839 | −13.764 | −30.929 | 1.00 | 36.48 |
| ATOM | 1243 | NH2 | ARG | A | 160 | −3.501 | −12.489 | −32.289 | 1.00 | 34.33 |
| ATOM | 1244 | C | ARG | A | 160 | −3.785 | −8.058 | −27.580 | 1.00 | 18.24 |
| ATOM | 1245 | O | ARG | A | 160 | −3.262 | −8.233 | −26.517 | 1.00 | 18.13 |
| ATOM | 1246 | N | ASN | A | 161 | −3.182 | −7.371 | −28.551 | 1.00 | 18.57 |
| ATOM | 1247 | CA | ASN | A | 161 | −1.875 | −6.717 | −28.289 | 1.00 | 18.40 |
| ATOM | 1248 | CB | ASN | A | 161 | −1.344 | −6.052 | −29.561 | 1.00 | 18.82 |
| ATOM | 1249 | CG | ASN | A | 161 | −0.772 | −7.055 | −30.549 | 1.00 | 20.68 |
| ATOM | 1250 | OD1 | ASN | A | 161 | −0.240 | −8.097 | −30.149 | 1.00 | 22.60 |
| ATOM | 1251 | ND2 | ASN | A | 161 | −0.883 | −6.751 | −31.837 | 1.00 | 19.81 |
| ATOM | 1252 | C | ASN | A | 161 | −1.946 | −5.656 | −27.182 | 1.00 | 18.36 |
| ATOM | 1253 | O | ASN | A | 161 | −1.078 | −5.581 | −26.313 | 1.00 | 17.42 |
| ATOM | 1254 | N | ASP | A | 162 | −2.982 | −4.816 | −27.233 | 1.00 | 17.86 |
| ATOM | 1255 | CA | ASP | A | 162 | −3.163 | −3.782 | −26.194 | 1.00 | 16.85 |
| ATOM | 1256 | CB | ASP | A | 162 | −4.293 | −2.821 | −26.586 | 1.00 | 16.71 |
| ATOM | 1257 | CG | ASP | A | 162 | −3.851 | −1.791 | −27.623 | 1.00 | 17.46 |
| ATOM | 1258 | OD1 | ASP | A | 162 | −2.648 | −1.440 | −27.681 | 1.00 | 16.98 |
| ATOM | 1259 | OD2 | ASP | A | 162 | −4.719 | −1.333 | −28.388 | 1.00 | 18.89 |
| ATOM | 1260 | C | ASP | A | 162 | −3.421 | −4.354 | −24.799 | 1.00 | 16.51 |
| ATOM | 1261 | O | ASP | A | 162 | −2.846 | −3.897 | −23.822 | 1.00 | 15.71 |
| ATOM | 1262 | N | LEU | A | 163 | −4.278 | −5.371 | −24.715 | 1.00 | 16.83 |
| ATOM | 1263 | CA | LEU | A | 163 | −4.532 | −6.071 | −23.459 | 1.00 | 16.55 |
| ATOM | 1264 | CB | LEU | A | 163 | −5.661 | −7.088 | −23.637 | 1.00 | 16.96 |
| ATOM | 1265 | CG | LEU | A | 163 | −7.030 | −6.506 | −23.975 | 1.00 | 19.29 |
| ATOM | 1266 | CD1 | LEU | A | 163 | −8.007 | −7.663 | −24.227 | 1.00 | 19.71 |
| ATOM | 1267 | CD2 | LEU | A | 163 | −7.484 | −5.631 | −22.818 | 1.00 | 21.19 |
| ATOM | 1268 | C | LEU | A | 163 | −3.279 | −6.750 | −22.883 | 1.00 | 16.19 |
| ATOM | 1269 | O | LEU | A | 163 | −3.035 | −6.690 | −21.688 | 1.00 | 15.47 |
| ATOM | 1270 | N | ASN | A | 164 | −2.495 | −7.401 | −23.748 | 1.00 | 16.85 |
| ATOM | 1271 | CA | ASN | A | 164 | −1.251 | −8.040 | −23.305 | 1.00 | 16.44 |
| ATOM | 1272 | CB | ASN | A | 164 | −0.602 | −8.836 | −24.450 | 1.00 | 17.06 |
| ATOM | 1273 | CG | ASN | A | 164 | −1.333 | −10.153 | −24.718 | 1.00 | 19.63 |
| ATOM | 1274 | OD1 | ASN | A | 164 | −2.274 | −10.513 | −23.982 | 1.00 | 20.97 |
| ATOM | 1275 | ND2 | ASN | A | 164 | −0.903 | −10.881 | −25.756 | 1.00 | 19.79 |
| ATOM | 1276 | C | ASN | A | 164 | −0.301 | −7.022 | −22.761 | 1.00 | 16.80 |
| ATOM | 1277 | O | ASN | A | 164 | 0.349 | −7.261 | −21.751 | 1.00 | 15.97 |
| ATOM | 1278 | N | TYR | A | 165 | −0.250 | −5.860 | −23.415 | 1.00 | 16.45 |
| ATOM | 1279 | CA | TYR | A | 165 | 0.573 | −4.744 | −22.930 | 1.00 | 16.15 |
| ATOM | 1280 | CB | TYR | A | 165 | 0.420 | −3.508 | −23.844 | 1.00 | 16.63 |
| ATOM | 1281 | CG | TYR | A | 165 | 1.286 | −2.356 | −23.391 | 1.00 | 16.41 |
| ATOM | 1282 | CD1 | TYR | A | 165 | 0.838 | −1.459 | −22.404 | 1.00 | 17.94 |
| ATOM | 1283 | CE1 | TYR | A | 165 | 1.651 | −0.402 | −21.958 | 1.00 | 17.84 |
| ATOM | 1284 | CZ | TYR | A | 165 | 2.916 | −0.223 | −22.517 | 1.00 | 18.36 |
| ATOM | 1285 | OH | TYR | A | 165 | 3.699 | 0.841 | −22.091 | 1.00 | 16.54 |
| ATOM | 1286 | CE2 | TYR | A | 165 | 3.383 | −1.105 | −23.502 | 1.00 | 16.73 |
| ATOM | 1287 | CD2 | TYR | A | 165 | 2.552 | −2.157 | −23.942 | 1.00 | 16.01 |
| ATOM | 1288 | C | TYR | A | 165 | 0.198 | −4.366 | −21.503 | 1.00 | 16.26 |
| ATOM | 1289 | O | TYR | A | 165 | 1.073 | −4.218 | −20.650 | 1.00 | 15.43 |
| ATOM | 1290 | N | VAL | A | 166 | −1.104 | −4.177 | −21.258 | 1.00 | 16.75 |
| ATOM | 1291 | CA | VAL | A | 166 | −1.600 | −3.786 | −19.933 | 1.00 | 17.39 |
| ATOM | 1292 | CB | VAL | A | 166 | −3.124 | −3.479 | −19.986 | 1.00 | 17.57 |
| ATOM | 1293 | CG1 | VAL | A | 166 | −3.712 | −3.197 | −18.582 | 1.00 | 19.25 |
| ATOM | 1294 | CG2 | VAL | A | 166 | −3.363 | −2.272 | −20.909 | 1.00 | 16.49 |
| ATOM | 1295 | C | VAL | A | 166 | −1.258 | −4.829 | −18.865 | 1.00 | 17.83 |
| ATOM | 1296 | O | VAL | A | 166 | −0.741 | −4.483 | −17.792 | 1.00 | 18.00 |
| ATOM | 1297 | N | ALA | A | 167 | −1.520 | −6.099 | −19.188 | 1.00 | 18.26 |
| ATOM | 1298 | CA | ALA | A | 167 | −1.233 | −7.218 | −18.285 | 1.00 | 18.92 |
| ATOM | 1299 | CB | ALA | A | 167 | −1.716 | −8.532 | −18.899 | 1.00 | 18.25 |
| ATOM | 1300 | C | ALA | A | 167 | 0.251 | −7.325 | −17.956 | 1.00 | 19.08 |
| ATOM | 1301 | O | ALA | A | 167 | 0.611 | −7.757 | −16.854 | 1.00 | 20.02 |
| ATOM | 1302 | N | GLN | A | 168 | 1.097 | −6.955 | −18.920 | 1.00 | 19.13 |
| ATOM | 1303 | CA | GLN | A | 168 | 2.558 | −7.022 | −18.749 | 1.00 | 19.21 |
| ATOM | 1304 | CB | GLN | A | 168 | 3.218 | −7.201 | −20.115 | 1.00 | 19.08 |
| ATOM | 1305 | CG | GLN | A | 168 | 4.739 | −7.373 | −20.053 | 1.00 | 20.55 |
| ATOM | 1306 | CD | GLN | A | 168 | 5.337 | −7.891 | −21.355 | 1.00 | 20.26 |
| ATOM | 1307 | OE1 | GLN | A | 168 | 4.634 | −8.378 | −22.238 | 1.00 | 22.69 |
| ATOM | 1308 | NE2 | GLN | A | 168 | 6.643 | −7.772 | −21.476 | 1.00 | 23.10 |
| ATOM | 1309 | C | GLN | A | 168 | 3.182 | −5.807 | −18.048 | 1.00 | 19.60 |
| ATOM | 1310 | O | GLN | A | 168 | 4.104 | −5.942 | −17.205 | 1.00 | 18.87 |
| ATOM | 1311 | N | TYR | A | 169 | 2.709 | −4.609 | −18.404 | 1.00 | 19.23 |
| ATOM | 1312 | CA | TYR | A | 169 | 3.399 | −3.377 | −18.011 | 1.00 | 19.45 |
| ATOM | 1313 | CB | TYR | A | 169 | 3.760 | −2.560 | −19.266 | 1.00 | 20.43 |
| ATOM | 1314 | CG | TYR | A | 169 | 4.773 | −3.203 | −20.203 | 1.00 | 21.30 |
| ATOM | 1315 | CD1 | TYR | A | 169 | 6.125 | −3.243 | −19.872 | 1.00 | 23.63 |
| ATOM | 1316 | CE1 | TYR | A | 169 | 7.065 | −3.822 | −20.723 | 1.00 | 24.59 |
| ATOM | 1317 | CZ | TYR | A | 169 | 6.651 | −4.359 | −21.926 | 1.00 | 23.11 |
| ATOM | 1318 | OH | TYR | A | 169 | 7.580 | −4.924 | −22.779 | 1.00 | 25.26 |
| ATOM | 1319 | CE2 | TYR | A | 169 | 5.309 | −4.330 | −22.288 | 1.00 | 22.49 |
| ATOM | 1320 | CD2 | TYR | A | 169 | 4.375 | −3.754 | −21.422 | 1.00 | 20.87 |
| ATOM | 1321 | C | TYR | A | 169 | 2.675 | −2.449 | −17.015 | 1.00 | 19.49 |
| ATOM | 1322 | O | TYR | A | 169 | 3.205 | −1.386 | −16.691 | 1.00 | 19.69 |
| ATOM | 1323 | N | TRP | A | 170 | 1.508 | −2.850 | −16.498 | 1.00 | 18.91 |
| ATOM | 1324 | CA | TRP | A | 170 | 0.735 | −1.981 | −15.588 | 1.00 | 19.29 |
| ATOM | 1325 | CB | TRP | A | 170 | −0.610 | −2.626 | −15.208 | 1.00 | 18.85 |
| ATOM | 1326 | CG | TRP | A | 170 | −0.489 | −3.743 | −14.215 | 1.00 | 21.04 |
| ATOM | 1327 | CD1 | TRP | A | 170 | −0.342 | −5.083 | −14.489 | 1.00 | 20.67 |
| ATOM | 1328 | NE1 | TRP | A | 170 | −0.259 | −5.793 | −13.317 | 1.00 | 22.09 |
| ATOM | 1329 | CE2 | TRP | A | 170 | −0.336 | −4.928 | −12.258 | 1.00 | 19.19 |
| ATOM | 1330 | CD2 | TRP | A | 170 | −0.481 | −3.621 | −12.789 | 1.00 | 20.35 |
| ATOM | 1331 | CE3 | TRP | A | 170 | −0.582 | −2.530 | −11.905 | 1.00 | 19.61 |
| ATOM | 1332 | CZ3 | TRP | A | 170 | −0.546 | −2.769 | −10.542 | 1.00 | 22.33 |
| ATOM | 1333 | CH2 | TRP | A | 170 | −0.404 | −4.090 | −10.038 | 1.00 | 21.08 |
| ATOM | 1334 | CZ2 | TRP | A | 170 | −0.297 | −5.179 | −10.884 | 1.00 | 20.76 |
| ATOM | 1335 | C | TRP | A | 170 | 1.526 | −1.592 | −14.336 | 1.00 | 19.20 |
| ATOM | 1336 | O | TRP | A | 170 | 1.395 | −0.475 | −13.808 | 1.00 | 19.24 |
| ATOM | 1337 | N | ASN | A | 171 | 2.371 | −2.504 | −13.858 | 1.00 | 19.13 |
| ATOM | 1338 | CA | ASN | A | 171 | 3.054 | −2.280 | −12.596 | 1.00 | 20.12 |
| ATOM | 1339 | CB | ASN | A | 171 | 3.178 | −3.603 | −11.820 | 1.00 | 20.59 |
| ATOM | 1340 | CG | ASN | A | 171 | 3.646 | −3.419 | −10.392 | 1.00 | 22.31 |
| ATOM | 1341 | OD1 | ASN | A | 171 | 4.531 | −4.155 | −9.938 | 1.00 | 23.68 |
| ATOM | 1342 | ND2 | ASN | A | 171 | 3.081 | −2.435 | −9.684 | 1.00 | 18.77 |
| ATOM | 1343 | C | ASN | A | 171 | 4.392 | −1.557 | −12.797 | 1.00 | 20.93 |
| ATOM | 1344 | O | ASN | A | 171 | 5.333 | −1.724 | −12.022 | 1.00 | 20.35 |
| ATOM | 1345 | N | GLN | A | 172 | 4.449 | −0.712 | −13.826 | 1.00 | 20.64 |
| ATOM | 1346 | CA | GLN | A | 172 | 5.644 | 0.061 | −14.156 | 1.00 | 22.31 |
| ATOM | 1347 | CB | GLN | A | 172 | 6.262 | −0.452 | −15.469 | 1.00 | 22.04 |
| ATOM | 1348 | CG | GLN | A | 172 | 6.784 | −1.895 | −15.312 | 1.00 | 25.79 |
| ATOM | 1349 | CD | GLN | A | 172 | 7.536 | −2.450 | −16.515 | 1.00 | 27.61 |
| ATOM | 1350 | OE1 | GLN | A | 172 | 8.276 | −1.735 | −17.214 | 1.00 | 35.65 |
| ATOM | 1351 | NE2 | GLN | A | 172 | 7.367 | −3.755 | −16.752 | 1.00 | 33.80 |
| ATOM | 1352 | C | GLN | A | 172 | 5.287 | 1.539 | −14.268 | 1.00 | 21.36 |
| ATOM | 1353 | O | GLN | A | 172 | 4.175 | 1.867 | −14.704 | 1.00 | 21.04 |
| ATOM | 1354 | N | THR | A | 173 | 6.209 | 2.417 | −13.871 | 1.00 | 19.73 |
| ATOM | 1355 | CA | THR | A | 173 | 5.948 | 3.871 | −13.928 | 1.00 | 20.07 |
| ATOM | 1356 | CB | THR | A | 173 | 7.001 | 4.703 | −13.168 | 1.00 | 19.48 |
| ATOM | 1357 | OG1 | THR | A | 173 | 8.300 | 4.427 | −13.707 | 1.00 | 21.56 |
| ATOM | 1358 | CG2 | THR | A | 173 | 6.988 | 4.375 | −11.690 | 1.00 | 20.86 |
| ATOM | 1359 | C | THR | A | 173 | 5.913 | 4.347 | −15.375 | 1.00 | 18.53 |
| ATOM | 1360 | O | THR | A | 173 | 6.395 | 3.665 | −16.292 | 1.00 | 18.48 |
| ATOM | 1361 | N | GLY | A | 174 | 5.345 | 5.528 | −15.582 | 1.00 | 18.38 |
| ATOM | 1362 | CA | GLY | A | 174 | 5.363 | 6.149 | −16.903 | 1.00 | 17.13 |
| ATOM | 1363 | C | GLY | A | 174 | 4.760 | 7.522 | −16.736 | 1.00 | 16.77 |
| ATOM | 1364 | O | GLY | A | 174 | 4.462 | 7.939 | −15.605 | 1.00 | 16.87 |
| ATOM | 1365 | N | PHE | A | 175 | 4.571 | 8.223 | −17.849 | 1.00 | 14.68 |
| ATOM | 1366 | CA | PHE | A | 175 | 4.004 | 9.577 | −17.776 | 1.00 | 14.68 |
| ATOM | 1367 | CB | PHE | A | 175 | 4.522 | 10.432 | −18.948 | 1.00 | 15.16 |
| ATOM | 1368 | CG | PHE | A | 175 | 5.943 | 10.847 | −18.756 | 1.00 | 15.28 |
| ATOM | 1369 | CD1 | PHE | A | 175 | 6.981 | 10.000 | −19.144 | 1.00 | 18.04 |
| ATOM | 1370 | CE1 | PHE | A | 175 | 8.313 | 10.359 | −18.915 | 1.00 | 19.53 |
| ATOM | 1371 | CZ | PHE | A | 175 | 8.609 | 11.582 | −18.278 | 1.00 | 19.12 |
| ATOM | 1372 | CE2 | PHE | A | 175 | 7.571 | 12.429 | −17.876 | 1.00 | 18.63 |
| ATOM | 1373 | CD2 | PHE | A | 175 | 6.247 | 12.054 | −18.113 | 1.00 | 17.67 |
| ATOM | 1374 | C | PHE | A | 175 | 2.483 | 9.584 | −17.655 | 1.00 | 14.11 |
| ATOM | 1375 | O | PHE | A | 175 | 1.799 | 8.683 | −18.175 | 1.00 | 14.32 |
| ATOM | 1376 | N | ASP | A | 176 | 1.972 | 10.591 | −16.938 | 1.00 | 14.79 |
| ATOM | 1377 | CA | ASP | A | 176 | 0.541 | 10.764 | −16.713 | 1.00 | 14.45 |
| ATOM | 1378 | CB | ASP | A | 176 | 0.297 | 11.661 | −15.506 | 1.00 | 13.51 |
| ATOM | 1379 | CG | ASP | A | 176 | 0.685 | 13.126 | −15.760 | 1.00 | 14.99 |
| ATOM | 1380 | OD1 | ASP | A | 176 | 1.774 | 13.399 | −16.329 | 1.00 | 14.32 |
| ATOM | 1381 | OD2 | ASP | A | 176 | −0.112 | 14.012 | −15.376 | 1.00 | 15.08 |
| ATOM | 1382 | C | ASP | A | 176 | −0.143 | 11.343 | −17.962 | 1.00 | 14.21 |
| ATOM | 1383 | O | ASP | A | 176 | 0.525 | 11.641 | −18.963 | 1.00 | 14.31 |
| ATOM | 1384 | N | LEU | A | 177 | −1.467 | 11.511 | −17.891 | 1.00 | 13.30 |
| ATOM | 1385 | CA | LEU | A | 177 | −2.235 | 11.981 | −19.048 | 1.00 | 13.44 |
| ATOM | 1386 | CB | LEU | A | 177 | −3.752 | 11.839 | −18.832 | 1.00 | 13.71 |
| ATOM | 1387 | CG | LEU | A | 177 | −4.483 | 12.896 | −18.012 | 1.00 | 14.11 |
| ATOM | 1388 | CD1 | LEU | A | 177 | −5.996 | 12.647 | −18.061 | 1.00 | 13.65 |
| ATOM | 1389 | CD2 | LEU | A | 177 | −4.007 | 12.922 | −16.553 | 1.00 | 14.74 |
| ATOM | 1390 | C | LEU | A | 177 | −1.884 | 13.424 | −19.452 | 1.00 | 13.51 |
| ATOM | 1391 | O | LEU | A | 177 | −2.131 | 13.813 | −20.600 | 1.00 | 13.74 |
| ATOM | 1392 | N | TRP | A | 178 | −1.319 | 14.206 | −18.521 | 1.00 | 12.53 |
| ATOM | 1393 | CA | TRP | A | 178 | −0.804 | 15.553 | −18.855 | 1.00 | 12.95 |
| ATOM | 1394 | CB | TRP | A | 178 | −0.890 | 16.507 | −17.660 | 1.00 | 12.67 |
| ATOM | 1395 | CG | TRP | A | 178 | −2.247 | 16.549 | −17.005 | 1.00 | 13.10 |
| ATOM | 1396 | CD1 | TRP | A | 178 | −2.504 | 16.508 | −15.662 | 1.00 | 13.10 |
| ATOM | 1397 | NE1 | TRP | A | 178 | −3.856 | 16.568 | −15.440 | 1.00 | 12.14 |
| ATOM | 1398 | CE2 | TRP | A | 178 | −4.501 | 16.646 | −16.646 | 1.00 | 13.00 |
| ATOM | 1399 | CD2 | TRP | A | 178 | −3.516 | 16.641 | −17.657 | 1.00 | 12.53 |
| ATOM | 1400 | CE3 | TRP | A | 178 | −3.919 | 16.715 | −19.002 | 1.00 | 12.38 |
| ATOM | 1401 | CZ3 | TRP | A | 178 | −5.309 | 16.813 | −19.290 | 1.00 | 13.91 |
| ATOM | 1402 | CH2 | TRP | A | 178 | −6.262 | 16.804 | −18.257 | 1.00 | 13.52 |
| ATOM | 1403 | CZ2 | TRP | A | 178 | −5.883 | 16.718 | −16.930 | 1.00 | 13.97 |
| ATOM | 1404 | C | TRP | A | 178 | 0.632 | 15.565 | −19.400 | 1.00 | 13.35 |
| ATOM | 1405 | O | TRP | A | 178 | 1.147 | 16.641 | −19.756 | 1.00 | 13.76 |
| ATOM | 1406 | N | GLU | A | 179 | 1.255 | 14.387 | −19.447 | 1.00 | 13.33 |
| ATOM | 1407 | CA | GLU | A | 179 | 2.532 | 14.151 | −20.117 | 1.00 | 13.32 |
| ATOM | 1408 | CB | GLU | A | 179 | 2.503 | 14.632 | −21.582 | 1.00 | 12.64 |
| ATOM | 1409 | CG | GLU | A | 179 | 1.165 | 14.344 | −22.280 | 1.00 | 13.03 |
| ATOM | 1410 | CD | GLU | A | 179 | 1.274 | 14.434 | −23.785 | 1.00 | 14.68 |
| ATOM | 1411 | OE1 | GLU | A | 179 | 0.895 | 15.478 | −24.340 | 1.00 | 15.98 |
| ATOM | 1412 | OE2 | GLU | A | 179 | 1.730 | 13.457 | −24.405 | 1.00 | 15.44 |
| ATOM | 1413 | C | GLU | A | 179 | 3.667 | 14.853 | −19.374 | 1.00 | 15.00 |
| ATOM | 1414 | O | GLU | A | 179 | 4.626 | 15.292 | −20.004 | 1.00 | 14.90 |
| ATOM | 1415 | N | GLU | A | 180 | 3.561 | 14.932 | −18.048 | 1.00 | 14.78 |
| ATOM | 1416 | CA | GLU | A | 180 | 4.476 | 15.745 | −17.246 | 1.00 | 16.76 |
| ATOM | 1417 | CB | GLU | A | 180 | 3.719 | 16.928 | −16.630 | 1.00 | 16.95 |
| ATOM | 1418 | CG | GLU | A | 180 | 3.282 | 17.972 | −17.654 | 1.00 | 18.69 |
| ATOM | 1419 | CD | GLU | A | 180 | 2.240 | 18.969 | −17.122 | 1.00 | 19.72 |
| ATOM | 1420 | OE1 | GLU | A | 180 | 1.587 | 18.715 | −16.077 | 1.00 | 19.00 |
| ATOM | 1421 | OE2 | GLU | A | 180 | 2.076 | 20.020 | −17.793 | 1.00 | 24.62 |
| ATOM | 1422 | C | GLU | A | 180 | 5.124 | 14.954 | −16.104 | 1.00 | 16.50 |
| ATOM | 1423 | O | GLU | A | 180 | 6.265 | 15.202 | −15.750 | 1.00 | 17.36 |
| ATOM | 1424 | N | VAL | A | 181 | 4.364 | 14.056 | −15.488 | 1.00 | 16.77 |
| ATOM | 1425 | CA | VAL | A | 181 | 4.775 | 13.426 | −14.218 | 1.00 | 16.87 |
| ATOM | 1426 | CB | VAL | A | 181 | 3.672 | 13.555 | −13.130 | 1.00 | 16.78 |
| ATOM | 1427 | CG1 | VAL | A | 181 | 4.030 | 12.732 | −11.893 | 1.00 | 18.56 |
| ATOM | 1428 | CG2 | VAL | A | 181 | 3.490 | 15.008 | −12.726 | 1.00 | 17.21 |
| ATOM | 1429 | C | VAL | A | 181 | 5.057 | 11.953 | −14.451 | 1.00 | 17.22 |
| ATOM | 1430 | O | VAL | A | 181 | 4.177 | 11.205 | −14.825 | 1.00 | 16.93 |
| ATOM | 1431 | N | ASN | A | 182 | 6.290 | 11.532 | −14.201 | 1.00 | 18.39 |
| ATOM | 1432 | CA | ASN | A | 182 | 6.674 | 10.123 | −14.394 | 1.00 | 18.57 |
| ATOM | 1433 | CB | ASN | A | 182 | 8.136 | 10.079 | −14.845 | 1.00 | 19.77 |
| ATOM | 1434 | CG | ASN | A | 182 | 8.665 | 8.669 | −15.056 | 1.00 | 23.96 |
| ATOM | 1435 | OD1 | ASN | A | 182 | 9.881 | 8.470 | −15.058 | 1.00 | 33.20 |
| ATOM | 1436 | ND2 | ASN | A | 182 | 7.794 | 7.706 | −15.258 | 1.00 | 23.63 |
| ATOM | 1437 | C | ASN | A | 182 | 6.440 | 9.375 | −13.073 | 1.00 | 18.31 |
| ATOM | 1438 | O | ASN | A | 182 | 7.132 | 9.621 | −12.087 | 1.00 | 18.80 |
| ATOM | 1439 | N | GLY | A | 183 | 5.436 | 8.508 | −13.034 | 1.00 | 16.83 |
| ATOM | 1440 | CA | GLY | A | 183 | 5.091 | 7.828 | −11.790 | 1.00 | 15.98 |
| ATOM | 1441 | C | GLY | A | 183 | 3.989 | 6.837 | −12.033 | 1.00 | 15.59 |
| ATOM | 1442 | O | GLY | A | 183 | 3.937 | 6.228 | −13.117 | 1.00 | 15.30 |
| ATOM | 1443 | N | SER | A | 184 | 3.119 | 6.670 | −11.035 | 1.00 | 14.95 |
| ATOM | 1444 | CA | SER | A | 184 | 1.927 | 5.823 | −11.151 | 1.00 | 15.45 |
| ATOM | 1445 | CB | SER | A | 184 | 1.844 | 4.792 | −10.017 | 1.00 | 15.76 |
| ATOM | 1446 | OG | SER | A | 184 | 2.998 | 3.935 | −10.027 | 1.00 | 17.33 |
| ATOM | 1447 | C | SER | A | 184 | 0.731 | 6.758 | −11.073 | 1.00 | 15.14 |
| ATOM | 1448 | O | SER | A | 184 | 0.646 | 7.546 | −10.148 | 1.00 | 15.83 |
| ATOM | 1449 | N | SER | A | 185 | −0.151 | 6.706 | −12.066 | 1.00 | 14.73 |
| ATOM | 1450 | CA | SER | A | 185 | −1.169 | 7.755 | −12.190 | 1.00 | 13.87 |
| ATOM | 1451 | CB | SER | A | 185 | −0.991 | 8.535 | −13.515 | 1.00 | 14.73 |
| ATOM | 1452 | OG | SER | A | 185 | −1.793 | 9.721 | −13.544 | 1.00 | 14.93 |
| ATOM | 1453 | C | SER | A | 185 | −2.551 | 7.140 | −12.127 | 1.00 | 13.49 |
| ATOM | 1454 | O | SER | A | 185 | −2.834 | 6.134 | −12.792 | 1.00 | 13.35 |
| ATOM | 1455 | N | PHE | A | 186 | −3.427 | 7.782 | −11.354 | 1.00 | 13.72 |
| ATOM | 1456 | CA | PHE | A | 186 | −4.764 | 7.275 | −11.092 | 1.00 | 13.43 |
| ATOM | 1457 | CB | PHE | A | 186 | −5.511 | 8.319 | −10.260 | 1.00 | 13.58 |
| ATOM | 1458 | CG | PHE | A | 186 | −6.807 | 7.839 | −9.662 | 1.00 | 13.52 |
| ATOM | 1459 | CD1 | PHE | A | 186 | −6.819 | 6.873 | −8.655 | 1.00 | 16.11 |
| ATOM | 1460 | CE1 | PHE | A | 186 | −8.004 | 6.489 | −8.036 | 1.00 | 17.80 |
| ATOM | 1461 | CZ | PHE | A | 186 | −9.214 | 7.062 | −8.442 | 1.00 | 16.18 |
| ATOM | 1462 | CE2 | PHE | A | 186 | −9.211 | 8.051 | −9.432 | 1.00 | 15.77 |
| ATOM | 1463 | CD2 | PHE | A | 186 | −8.003 | 8.435 | −10.030 | 1.00 | 14.62 |
| ATOM | 1464 | C | PHE | A | 186 | −5.552 | 6.946 | −12.372 | 1.00 | 13.57 |
| ATOM | 1465 | O | PHE | A | 186 | −6.053 | 5.839 | −12.524 | 1.00 | 13.36 |
| ATOM | 1466 | N | PHE | A | 187 | −5.693 | 7.927 | −13.267 | 1.00 | 12.53 |
| ATOM | 1467 | CA | PHE | A | 187 | −6.416 | 7.762 | −14.527 | 1.00 | 12.84 |
| ATOM | 1468 | CB | PHE | A | 187 | −6.284 | 9.056 | −15.356 | 1.00 | 11.69 |
| ATOM | 1469 | CG | PHE | A | 187 | −6.949 | 9.016 | −16.711 | 1.00 | 13.25 |
| ATOM | 1470 | CD1 | PHE | A | 187 | −8.284 | 9.338 | −16.855 | 1.00 | 12.86 |
| ATOM | 1471 | CE1 | PHE | A | 187 | −8.893 | 9.342 | −18.102 | 1.00 | 14.12 |
| ATOM | 1472 | CZ | PHE | A | 187 | −8.139 | 9.041 | −19.236 | 1.00 | 14.05 |
| ATOM | 1473 | CE2 | PHE | A | 187 | −6.806 | 8.721 | −19.111 | 1.00 | 14.30 |
| ATOM | 1474 | CD2 | PHE | A | 187 | −6.206 | 8.711 | −17.857 | 1.00 | 15.10 |
| ATOM | 1475 | C | PHE | A | 187 | −5.887 | 6.563 | −15.318 | 1.00 | 12.69 |
| ATOM | 1476 | O | PHE | A | 187 | −6.666 | 5.837 | −15.932 | 1.00 | 14.00 |
| ATOM | 1477 | N | THR | A | 188 | −4.571 | 6.357 | −15.294 | 1.00 | 12.97 |
| ATOM | 1478 | CA | THR | A | 188 | −3.938 | 5.302 | −16.084 | 1.00 | 13.65 |
| ATOM | 1479 | CB | THR | A | 188 | −2.411 | 5.541 | −16.104 | 1.00 | 13.69 |
| ATOM | 1480 | OG1 | THR | A | 188 | −2.158 | 6.789 | −16.753 | 1.00 | 15.37 |
| ATOM | 1481 | CG2 | THR | A | 188 | −1.648 | 4.432 | −16.833 | 1.00 | 13.24 |
| ATOM | 1482 | C | THR | A | 188 | −4.284 | 3.929 | −15.478 | 1.00 | 14.12 |
| ATOM | 1483 | O | THR | A | 188 | −4.766 | 3.039 | −16.173 | 1.00 | 14.40 |
| ATOM | 1484 | N | VAL | A | 189 | −4.066 | 3.798 | −14.173 | 1.00 | 13.34 |
| ATOM | 1485 | CA | VAL | A | 189 | −4.348 | 2.543 | −13.446 | 1.00 | 14.76 |
| ATOM | 1486 | CB | VAL | A | 189 | −3.893 | 2.612 | −11.958 | 1.00 | 14.90 |
| ATOM | 1487 | CG1 | VAL | A | 189 | −4.331 | 1.334 | −11.186 | 1.00 | 16.95 |
| ATOM | 1488 | CG2 | VAL | A | 189 | −2.374 | 2.799 | −11.865 | 1.00 | 15.31 |
| ATOM | 1489 | C | VAL | A | 189 | −5.836 | 2.167 | −13.560 | 1.00 | 14.48 |
| ATOM | 1490 | O | VAL | A | 189 | −6.159 | 1.024 | −13.853 | 1.00 | 14.65 |
| ATOM | 1491 | N | ALA | A | 190 | −6.732 | 3.146 | −13.372 | 1.00 | 13.77 |
| ATOM | 1492 | CA | ALA | A | 190 | −8.171 | 2.858 | −13.351 | 1.00 | 13.46 |
| ATOM | 1493 | CB | ALA | A | 190 | −8.996 | 4.128 | −12.922 | 1.00 | 12.74 |
| ATOM | 1494 | C | ALA | A | 190 | −8.614 | 2.388 | −14.706 | 1.00 | 13.27 |
| ATOM | 1495 | O | ALA | A | 190 | −9.432 | 1.479 | −14.815 | 1.00 | 13.24 |
| ATOM | 1496 | N | ASN | A | 191 | −8.093 | 3.017 | −15.760 | 1.00 | 12.50 |
| ATOM | 1497 | CA | ASN | A | 191 | −8.438 | 2.598 | −17.127 | 1.00 | 13.01 |
| ATOM | 1498 | CB | ASN | A | 191 | −8.122 | 3.707 | −18.137 | 1.00 | 12.65 |
| ATOM | 1499 | CG | ASN | A | 191 | −9.191 | 4.781 | −18.118 | 1.00 | 14.08 |
| ATOM | 1500 | OD1 | ASN | A | 191 | −10.319 | 4.541 | −18.554 | 1.00 | 16.39 |
| ATOM | 1501 | ND2 | ASN | A | 191 | −8.857 | 5.955 | −17.583 | 1.00 | 17.21 |
| ATOM | 1502 | C | ASN | A | 191 | −7.815 | 1.259 | −17.521 | 1.00 | 13.93 |
| ATOM | 1503 | O | ASN | A | 191 | −8.412 | 0.490 | −18.270 | 1.00 | 13.87 |
| ATOM | 1504 | N | GLN | A | 192 | −6.636 | 0.995 | −16.980 | 1.00 | 14.06 |
| ATOM | 1505 | CA | GLN | A | 192 | −5.988 | −0.311 | −17.139 | 1.00 | 14.96 |
| ATOM | 1506 | CB | GLN | A | 192 | −4.575 | −0.274 | −16.552 | 1.00 | 14.33 |
| ATOM | 1507 | CG | GLN | A | 192 | −3.555 | 0.435 | −17.500 | 1.00 | 13.64 |
| ATOM | 1508 | CD | GLN | A | 192 | −2.206 | 0.635 | −16.857 | 1.00 | 15.33 |
| ATOM | 1509 | OE1 | GLN | A | 192 | −2.074 | 0.568 | −15.646 | 1.00 | 15.48 |
| ATOM | 1510 | NE2 | GLN | A | 192 | −1.182 | 0.925 | −17.682 | 1.00 | 16.10 |
| ATOM | 1511 | C | GLN | A | 192 | −6.855 | −1.411 | −16.519 | 1.00 | 15.11 |
| ATOM | 1512 | O | GLN | A | 192 | −7.076 | −2.457 | −17.141 | 1.00 | 16.04 |
| ATOM | 1513 | N | HIS | A | 193 | −7.398 | −1.140 | −15.329 | 1.00 | 15.81 |
| ATOM | 1514 | CA | HIS | A | 193 | −8.314 | −2.069 | −14.668 | 1.00 | 16.01 |
| ATOM | 1515 | CB | HIS | A | 193 | −8.746 | −1.586 | −13.281 | 1.00 | 16.72 |
| ATOM | 1516 | CG | HIS | A | 193 | −9.806 | −2.454 | −12.669 | 1.00 | 17.39 |
| ATOM | 1517 | ND1 | HIS | A | 193 | −11.113 | −2.039 | −12.505 | 1.00 | 18.05 |
| ATOM | 1518 | CE1 | HIS | A | 193 | −11.821 | −3.028 | −11.983 | 1.00 | 18.70 |
| ATOM | 1519 | NE2 | HIS | A | 193 | −11.023 | −4.071 | −11.814 | 1.00 | 17.20 |
| ATOM | 1520 | CD2 | HIS | A | 193 | −9.758 | −3.739 | −12.242 | 1.00 | 18.04 |
| ATOM | 1521 | C | HIS | A | 193 | −9.536 | −2.343 | −15.521 | 1.00 | 15.85 |
| ATOM | 1522 | O | HIS | A | 193 | −9.898 | −3.501 | −15.732 | 1.00 | 15.70 |
| ATOM | 1523 | N | ARG | A | 194 | −10.185 | −1.285 | −15.995 | 1.00 | 15.27 |
| ATOM | 1524 | CA | ARG | A | 194 | −11.349 | −1.437 | −16.852 | 1.00 | 15.29 |
| ATOM | 1525 | CB | ARG | A | 194 | −11.922 | −0.073 | −17.234 | 1.00 | 14.30 |
| ATOM | 1526 | CG | ARG | A | 194 | −13.029 | −0.212 | −18.239 | 1.00 | 14.46 |
| ATOM | 1527 | CD | ARG | A | 194 | −13.614 | 1.102 | −18.723 | 1.00 | 15.43 |
| ATOM | 1528 | NE | ARG | A | 194 | −14.589 | 0.780 | −19.767 | 1.00 | 15.58 |
| ATOM | 1529 | CZ | ARG | A | 194 | −15.624 | 1.539 | −20.125 | 1.00 | 17.92 |
| ATOM | 1530 | NH1 | ARG | A | 194 | −15.815 | 2.744 | −19.576 | 1.00 | 14.60 |
| ATOM | 1531 | NH2 | ARG | A | 194 | −16.451 | 1.095 | −21.060 | 1.00 | 16.15 |
| ATOM | 1532 | C | ARG | A | 194 | −11.047 | −2.258 | −18.111 | 1.00 | 15.74 |
| ATOM | 1533 | O | ARG | A | 194 | −11.842 | −3.120 | −18.504 | 1.00 | 15.56 |
| ATOM | 1534 | N | ALA | A | 195 | −9.918 | −1.967 | −18.758 | 1.00 | 15.60 |
| ATOM | 1535 | CA | ALA | A | 195 | −9.562 | −2.638 | −20.004 | 1.00 | 15.90 |
| ATOM | 1536 | CB | ALA | A | 195 | −8.254 | −2.042 | −20.591 | 1.00 | 15.40 |
| ATOM | 1537 | C | ALA | A | 195 | −9.436 | −4.150 | −19.798 | 1.00 | 15.65 |
| ATOM | 1538 | O | ALA | A | 195 | −9.959 | −4.929 | −20.610 | 1.00 | 16.79 |
| ATOM | 1539 | N | LEU | A | 196 | −8.763 | −4.550 | −18.721 | 1.00 | 16.36 |
| ATOM | 1540 | CA | LEU | A | 196 | −8.552 | −5.976 | −18.423 | 1.00 | 17.02 |
| ATOM | 1541 | CB | LEU | A | 196 | −7.625 | −6.126 | −17.235 | 1.00 | 16.96 |
| ATOM | 1542 | CG | LEU | A | 196 | −6.167 | −5.744 | −17.532 | 1.00 | 16.96 |
| ATOM | 1543 | CD1 | LEU | A | 196 | −5.375 | −5.857 | −16.252 | 1.00 | 18.93 |
| ATOM | 1544 | CD2 | LEU | A | 196 | −5.590 | −6.636 | −18.630 | 1.00 | 20.38 |
| ATOM | 1545 | C | LEU | A | 196 | −9.877 | −6.685 | −18.167 | 1.00 | 17.92 |
| ATOM | 1546 | O | LEU | A | 196 | −10.102 | −7.795 | −18.643 | 1.00 | 18.98 |
| ATOM | 1547 | N | VAL | A | 197 | −10.779 | −6.014 | −17.454 | 1.00 | 18.51 |
| ATOM | 1548 | CA | VAL | A | 197 | −12.112 | −6.560 | −17.181 | 1.00 | 18.81 |
| ATOM | 1549 | CB | VAL | A | 197 | −12.875 | −5.702 | −16.130 | 1.00 | 18.26 |
| ATOM | 1550 | CG1 | VAL | A | 197 | −14.340 | −6.173 | −15.994 | 1.00 | 21.18 |
| ATOM | 1551 | CG2 | VAL | A | 197 | −12.149 | −5.784 | −14.778 | 1.00 | 19.79 |
| ATOM | 1552 | C | VAL | A | 197 | −12.924 | −6.779 | −18.462 | 1.00 | 19.05 |
| ATOM | 1553 | O | VAL | A | 197 | −13.456 | −7.884 | −18.693 | 1.00 | 18.62 |
| ATOM | 1554 | N | GLU | A | 198 | −13.010 | −5.752 | −19.308 | 1.00 | 18.43 |
| ATOM | 1555 | CA | GLU | A | 198 | −13.747 | −5.873 | −20.556 | 1.00 | 19.38 |
| ATOM | 1556 | CB | GLU | A | 198 | −13.849 | −4.517 | −21.241 | 1.00 | 19.38 |
| ATOM | 1557 | CG | GLU | A | 198 | −14.609 | −3.530 | −20.417 | 1.00 | 20.22 |
| ATOM | 1558 | CD | GLU | A | 198 | −15.334 | −2.537 | −21.298 | 1.00 | 22.66 |
| ATOM | 1559 | OE1 | GLU | A | 198 | −16.313 | −2.940 | −21.940 | 1.00 | 22.16 |
| ATOM | 1560 | OE2 | GLU | A | 198 | −14.924 | −1.369 | −21.342 | 1.00 | 22.92 |
| ATOM | 1561 | C | GLU | A | 198 | −13.094 | −6.861 | −21.509 | 1.00 | 19.78 |
| ATOM | 1562 | O | GLU | A | 198 | −13.780 | −7.506 | −22.303 | 1.00 | 20.29 |
| ATOM | 1563 | N | GLY | A | 199 | −11.770 | −6.944 | −21.435 | 1.00 | 19.78 |
| ATOM | 1564 | CA | GLY | A | 199 | −10.998 | −7.823 | −22.314 | 1.00 | 20.88 |
| ATOM | 1565 | C | GLY | A | 199 | −11.288 | −9.285 | −21.986 | 1.00 | 21.53 |
| ATOM | 1566 | O | GLY | A | 199 | −11.546 | −10.083 | −22.879 | 1.00 | 22.36 |
| ATOM | 1567 | N | ALA | A | 200 | −11.256 | −9.615 | −20.702 | 1.00 | 21.79 |
| ATOM | 1568 | CA | ALA | A | 200 | −11.605 | −10.956 | −20.234 | 1.00 | 22.44 |
| ATOM | 1569 | CB | ALA | A | 200 | −11.463 | −11.038 | −18.728 | 1.00 | 22.21 |
| ATOM | 1570 | C | ALA | A | 200 | −13.016 | −11.329 | −20.696 | 1.00 | 22.54 |
| ATOM | 1571 | O | ALA | A | 200 | −13.237 | −12.419 | −21.214 | 1.00 | 22.25 |
| ATOM | 1572 | N | THR | A | 201 | −13.965 | −10.403 | −20.573 | 1.00 | 22.56 |
| ATOM | 1573 | CA | THR | A | 201 | −15.345 | −10.671 | −20.989 | 1.00 | 22.77 |
| ATOM | 1574 | CB | THR | A | 201 | −16.302 | −9.527 | −20.551 | 1.00 | 22.83 |
| ATOM | 1575 | OG1 | THR | A | 201 | −16.219 | −9.387 | −19.134 | 1.00 | 24.92 |
| ATOM | 1576 | CG2 | THR | A | 201 | −17.756 | −9.819 | −20.929 | 1.00 | 23.76 |
| ATOM | 1577 | C | THR | A | 201 | −15.435 | −10.905 | −22.485 | 1.00 | 22.78 |
| ATOM | 1578 | O | THR | A | 201 | −16.099 | −11.851 | −22.925 | 1.00 | 22.95 |
| ATOM | 1579 | N | LEU | A | 202 | −14.760 | −10.069 | −23.275 | 1.00 | 21.78 |
| ATOM | 1580 | CA | LEU | A | 202 | −14.805 | −10.236 | −24.717 | 1.00 | 22.62 |
| ATOM | 1581 | CB | LEU | A | 202 | −14.149 | −9.055 | −25.434 | 1.00 | 22.14 |
| ATOM | 1582 | CG | LEU | A | 202 | −14.142 | −9.107 | −26.964 | 1.00 | 23.10 |
| ATOM | 1583 | CD1 | LEU | A | 202 | −15.544 | −9.198 | −27.564 | 1.00 | 24.20 |
| ATOM | 1584 | CD2 | LEU | A | 202 | −13.346 | −7.938 | −27.570 | 1.00 | 22.53 |
| ATOM | 1585 | C | LEU | A | 202 | −14.139 | −11.552 | −25.151 | 1.00 | 23.15 |
| ATOM | 1586 | O | LEU | A | 202 | −14.649 | −12.245 | −26.036 | 1.00 | 22.90 |
| ATOM | 1587 | N | ALA | A | 203 | −13.019 | −11.883 | −24.510 | 1.00 | 23.38 |
| ATOM | 1588 | CA | ALA | A | 203 | −12.300 | −13.129 | −24.787 | 1.00 | 24.07 |
| ATOM | 1589 | CB | ALA | A | 203 | −11.076 | −13.229 | −23.913 | 1.00 | 23.57 |
| ATOM | 1590 | C | ALA | A | 203 | −13.211 | −14.354 | −24.569 | 1.00 | 24.38 |
| ATOM | 1591 | O | ALA | A | 203 | −13.264 | −15.244 | −25.411 | 1.00 | 25.21 |
| ATOM | 1592 | N | ALA | A | 204 | −13.920 | −14.363 | −23.447 | 1.00 | 25.20 |
| ATOM | 1593 | CA | ALA | A | 204 | −14.849 | −15.442 | −23.093 | 1.00 | 26.63 |
| ATOM | 1594 | CB | ALA | A | 204 | −15.450 | −15.186 | −21.727 | 1.00 | 26.12 |
| ATOM | 1595 | C | ALA | A | 204 | −15.939 | −15.583 | −24.150 | 1.00 | 27.48 |
| ATOM | 1596 | O | ALA | A | 204 | −16.267 | −16.687 | −24.564 | 1.00 | 28.39 |
| ATOM | 1597 | N | THR | A | 205 | −16.494 | −14.461 | −24.593 | 1.00 | 27.71 |
| ATOM | 1598 | CA | THR | A | 205 | −17.497 | −14.470 | −25.652 | 1.00 | 28.39 |
| ATOM | 1599 | CB | THR | A | 205 | −18.088 | −13.051 | −25.855 | 1.00 | 28.42 |
| ATOM | 1600 | OG1 | THR | A | 205 | −18.669 | −12.631 | −24.622 | 1.00 | 29.32 |
| ATOM | 1601 | CG2 | THR | A | 205 | −19.150 | −13.051 | −26.932 | 1.00 | 27.32 |
| ATOM | 1602 | C | THR | A | 205 | −16.968 | −15.004 | −26.981 | 1.00 | 28.72 |
| ATOM | 1603 | O | THR | A | 205 | −17.697 | −15.690 | −27.719 | 1.00 | 29.06 |
| ATOM | 1604 | N | LEU | A | 206 | −15.712 | −14.698 | −27.288 | 1.00 | 28.58 |
| ATOM | 1605 | CA | LEU | A | 206 | −15.122 | −15.122 | −28.539 | 1.00 | 29.40 |
| ATOM | 1606 | CB | LEU | A | 206 | −14.034 | −14.144 | −29.001 | 1.00 | 29.40 |
| ATOM | 1607 | CG | LEU | A | 206 | −14.438 | −12.694 | −29.322 | 1.00 | 29.97 |
| ATOM | 1608 | CD1 | LEU | A | 206 | −13.212 | −11.899 | −29.755 | 1.00 | 30.30 |
| ATOM | 1609 | CD2 | LEU | A | 206 | −15.561 | −12.629 | −30.375 | 1.00 | 29.58 |
| ATOM | 1610 | C | LEU | A | 206 | −14.540 | −16.538 | −28.489 | 1.00 | 29.55 |
| ATOM | 1611 | O | LEU | A | 206 | −14.118 | −17.054 | −29.521 | 1.00 | 30.16 |
| ATOM | 1612 | N | GLY | A | 207 | −14.500 | −17.145 | −27.307 | 1.00 | 30.30 |
| ATOM | 1613 | CA | GLY | A | 207 | −13.786 | −18.419 | −27.122 | 1.00 | 30.91 |
| ATOM | 1614 | C | GLY | A | 207 | −12.294 | −18.274 | −27.375 | 1.00 | 31.55 |
| ATOM | 1615 | O | GLY | A | 207 | −11.654 | −19.173 | −27.935 | 1.00 | 31.31 |
| ATOM | 1616 | N | GLN | A | 208 | −11.746 | −17.115 | −26.989 | 1.00 | 31.08 |
| ATOM | 1617 | CA | GLN | A | 208 | −10.311 | −16.877 | −27.031 | 1.00 | 31.10 |
| ATOM | 1618 | CB | GLN | A | 208 | −9.999 | −15.540 | −27.703 | 1.00 | 31.08 |
| ATOM | 1619 | CG | GLN | A | 208 | −10.451 | −15.455 | −29.142 | 1.00 | 33.86 |
| ATOM | 1620 | CD | GLN | A | 208 | −9.469 | −16.059 | −30.126 | 1.00 | 38.19 |
| ATOM | 1621 | OE1 | GLN | A | 208 | −9.686 | −15.999 | −31.335 | 1.00 | 41.96 |
| ATOM | 1622 | NE2 | GLN | A | 208 | −8.386 | −16.633 | −29.626 | 1.00 | 38.96 |
| ATOM | 1623 | C | GLN | A | 208 | −9.765 | −16.909 | −25.611 | 1.00 | 30.45 |
| ATOM | 1624 | O | GLN | A | 208 | −10.516 | −17.048 | −24.658 | 1.00 | 30.63 |
| ATOM | 1625 | N | SER | A | 209 | −8.451 | −16.816 | −25.469 | 1.00 | 29.96 |
| ATOM | 1626 | CA | SER | A | 209 | −7.841 | −16.898 | −24.160 | 1.00 | 30.04 |
| ATOM | 1627 | CB | SER | A | 209 | −6.382 | −17.343 | −24.297 | 1.00 | 30.04 |
| ATOM | 1628 | OG | SER | A | 209 | −5.763 | −17.371 | −23.030 | 1.00 | 32.75 |
| ATOM | 1629 | C | SER | A | 209 | −7.948 | −15.564 | −23.409 | 1.00 | 29.53 |
| ATOM | 1630 | O | SER | A | 209 | −7.493 | −14.532 | −23.908 | 1.00 | 29.85 |
| ATOM | 1631 | N | GLY | A | 210 | −8.545 | −15.594 | −22.216 | 1.00 | 28.41 |
| ATOM | 1632 | CA | GLY | A | 210 | −8.745 | −14.388 | −21.401 | 1.00 | 27.16 |
| ATOM | 1633 | C | GLY | A | 210 | −8.344 | −14.480 | −19.938 | 1.00 | 26.83 |
| ATOM | 1634 | O | GLY | A | 210 | −8.425 | −13.498 | −19.203 | 1.00 | 26.61 |
| ATOM | 1635 | N | SER | A | 211 | −7.888 | −15.648 | −19.497 | 1.00 | 25.85 |
| ATOM | 1636 | CA | SER | A | 211 | −7.651 | −15.867 | −18.067 | 1.00 | 25.26 |
| ATOM | 1637 | CB | SER | A | 211 | −7.401 | −17.353 | −17.783 | 1.00 | 25.87 |
| ATOM | 1638 | OG | SER | A | 211 | −6.315 | −17.789 | −18.573 | 1.00 | 26.62 |
| ATOM | 1639 | C | SER | A | 211 | −6.509 | −15.026 | −17.498 | 1.00 | 24.55 |
| ATOM | 1640 | O | SER | A | 211 | −6.542 | −14.676 | −16.311 | 1.00 | 24.46 |
| ATOM | 1641 | N | ALA | A | 212 | −5.505 | −14.712 | −18.323 | 1.00 | 23.56 |
| ATOM | 1642 | CA | ALA | A | 212 | −4.423 | −13.816 | −17.906 | 1.00 | 23.42 |
| ATOM | 1643 | CB | ALA | A | 212 | −3.417 | −13.622 | −19.031 | 1.00 | 23.66 |
| ATOM | 1644 | C | ALA | A | 212 | −4.999 | −12.450 | −17.496 | 1.00 | 23.54 |
| ATOM | 1645 | O | ALA | A | 212 | −4.566 | −11.848 | −16.513 | 1.00 | 24.00 |
| ATOM | 1646 | N | TYR | A | 213 | −5.970 | −11.979 | −18.271 | 1.00 | 22.79 |
| ATOM | 1647 | CA | TYR | A | 213 | −6.594 | −10.676 | −18.017 | 1.00 | 22.18 |
| ATOM | 1648 | CB | TYR | A | 213 | −7.453 | −10.241 | −19.193 | 1.00 | 21.74 |
| ATOM | 1649 | CG | TYR | A | 213 | −6.761 | −10.345 | −20.515 | 1.00 | 20.05 |
| ATOM | 1650 | CD1 | TYR | A | 213 | −7.461 | −10.761 | −21.637 | 1.00 | 20.58 |
| ATOM | 1651 | CE1 | TYR | A | 213 | −6.854 | −10.854 | −22.868 | 1.00 | 21.95 |
| ATOM | 1652 | CZ | TYR | A | 213 | −5.503 | −10.545 | −22.988 | 1.00 | 20.62 |
| ATOM | 1653 | OH | TYR | A | 213 | −4.930 | −10.668 | −24.220 | 1.00 | 21.72 |
| ATOM | 1654 | CE2 | TYR | A | 213 | −4.758 | −10.149 | −21.888 | 1.00 | 19.76 |
| ATOM | 1655 | CD2 | TYR | A | 213 | −5.400 | −10.038 | −20.647 | 1.00 | 20.61 |
| ATOM | 1656 | C | TYR | A | 213 | −7.423 | −10.710 | −16.758 | 1.00 | 23.06 |
| ATOM | 1657 | O | TYR | A | 213 | −7.320 | −9.804 | −15.939 | 1.00 | 22.56 |
| ATOM | 1658 | N | SER | A | 214 | −8.226 | −11.767 | −16.578 | 1.00 | 23.15 |
| ATOM | 1659 | CA | SER | A | 214 | −9.064 | −11.832 | −15.392 | 1.00 | 23.90 |
| ATOM | 1660 | CB | SER | A | 214 | −10.244 | −12.798 | −15.580 | 1.00 | 24.54 |
| ATOM | 1661 | OG | SER | A | 214 | −9.776 | −14.085 | −15.939 | 1.00 | 27.95 |
| ATOM | 1662 | C | SER | A | 214 | −8.259 | −12.122 | −14.122 | 1.00 | 23.64 |
| ATOM | 1663 | O | SER | A | 214 | −8.676 | −11.762 | −13.026 | 1.00 | 23.43 |
| ATOM | 1664 | N | SER | A | 215 | −7.095 | −12.743 | −14.248 | 1.00 | 23.82 |
| ATOM | 1665 | CA | SER | A | 215 | −6.295 | −12.970 | −13.050 | 1.00 | 24.66 |
| ATOM | 1666 | CB | SER | A | 215 | −5.390 | −14.205 | −13.200 | 1.00 | 25.70 |
| ATOM | 1667 | OG | SER | A | 215 | −4.267 | −13.914 | −14.004 | 1.00 | 29.15 |
| ATOM | 1668 | C | SER | A | 215 | −5.491 | −11.739 | −12.610 | 1.00 | 23.98 |
| ATOM | 1669 | O | SER | A | 215 | −5.217 | −11.561 | −11.421 | 1.00 | 24.09 |
| ATOM | 1670 | N | VAL | A | 216 | −5.115 | −10.894 | −13.566 | 1.00 | 22.89 |
| ATOM | 1671 | CA | VAL | A | 216 | −4.347 | −9.679 | −13.272 | 1.00 | 22.50 |
| ATOM | 1672 | CB | VAL | A | 216 | −3.442 | −9.296 | −14.493 | 1.00 | 22.52 |
| ATOM | 1673 | CG1 | VAL | A | 216 | −2.855 | −7.888 | −14.369 | 1.00 | 24.11 |
| ATOM | 1674 | CG2 | VAL | A | 216 | −2.296 | −10.317 | −14.652 | 1.00 | 22.49 |
| ATOM | 1675 | C | VAL | A | 216 | −5.256 | −8.520 | −12.801 | 1.00 | 21.88 |
| ATOM | 1676 | O | VAL | A | 216 | −4.869 | −7.745 | −11.936 | 1.00 | 21.84 |
| ATOM | 1677 | N | ALA | A | 217 | −6.475 | −8.440 | −13.332 | 1.00 | 21.86 |
| ATOM | 1678 | CA | ALA | A | 217 | −7.374 | −7.303 | −13.050 | 1.00 | 21.59 |
| ATOM | 1679 | CB | ALA | A | 217 | −8.721 | −7.479 | −13.760 | 1.00 | 21.26 |
| ATOM | 1680 | C | ALA | A | 217 | −7.571 | −6.968 | −11.558 | 1.00 | 21.55 |
| ATOM | 1681 | O | ALA | A | 217 | −7.447 | −5.804 | −11.165 | 1.00 | 21.20 |
| ATOM | 1682 | N | PRO | A | 218 | −7.842 | −7.988 | −10.701 | 1.00 | 21.95 |
| ATOM | 1683 | CA | PRO | A | 218 | −8.030 | −7.700 | −9.282 | 1.00 | 21.59 |
| ATOM | 1684 | CB | PRO | A | 218 | −8.283 | −9.104 | −8.670 | 1.00 | 22.29 |
| ATOM | 1685 | CG | PRO | A | 218 | −8.789 | −9.905 | −9.789 | 1.00 | 22.61 |
| ATOM | 1686 | CD | PRO | A | 218 | −7.966 | −9.435 | −10.963 | 1.00 | 22.11 |
| ATOM | 1687 | C | PRO | A | 218 | −6.798 | −7.065 | −8.634 | 1.00 | 21.27 |
| ATOM | 1688 | O | PRO | A | 218 | −6.928 | −6.299 | −7.680 | 1.00 | 20.92 |
| ATOM | 1689 | N | GLN | A | 219 | −5.608 | −7.386 | −9.141 | 1.00 | 21.17 |
| ATOM | 1690 | CA | GLN | A | 219 | −4.378 | −6.786 | −8.609 | 1.00 | 21.51 |
| ATOM | 1691 | CB | GLN | A | 219 | −3.149 | −7.569 | −9.084 | 1.00 | 22.72 |
| ATOM | 1692 | CG | GLN | A | 219 | −3.113 | −8.985 | −8.516 | 1.00 | 24.90 |
| ATOM | 1693 | CD | GLN | A | 219 | −3.323 | −8.982 | −7.015 | 1.00 | 29.57 |
| ATOM | 1694 | OE1 | GLN | A | 219 | −2.715 | −8.188 | −6.288 | 1.00 | 31.58 |
| ATOM | 1695 | NE2 | GLN | A | 219 | −4.207 | −9.843 | −6.545 | 1.00 | 33.22 |
| ATOM | 1696 | C | GLN | A | 219 | −4.240 | −5.301 | −8.996 | 1.00 | 21.04 |
| ATOM | 1697 | O | GLN | A | 219 | −3.687 | −4.490 | −8.229 | 1.00 | 21.07 |
| ATOM | 1698 | N | VAL | A | 220 | −4.728 | −4.973 | −10.187 | 1.00 | 20.00 |
| ATOM | 1699 | CA | VAL | A | 220 | −4.746 | −3.577 | −10.630 | 1.00 | 19.34 |
| ATOM | 1700 | CB | VAL | A | 220 | −5.098 | −3.456 | −12.128 | 1.00 | 19.63 |
| ATOM | 1701 | CG1 | VAL | A | 220 | −4.991 | −2.000 | −12.581 | 1.00 | 19.15 |
| ATOM | 1702 | CG2 | VAL | A | 220 | −4.162 | −4.342 | −12.974 | 1.00 | 17.97 |
| ATOM | 1703 | C | VAL | A | 220 | −5.730 | −2.809 | −9.737 | 1.00 | 19.65 |
| ATOM | 1704 | O | VAL | A | 220 | −5.419 | −1.728 | −9.257 | 1.00 | 18.97 |
| ATOM | 1705 | N | LEU | A | 221 | −6.903 | −3.391 | −9.490 | 1.00 | 20.12 |
| ATOM | 1706 | CA | LEU | A | 221 | −7.895 | −2.776 | −8.620 | 1.00 | 20.83 |
| ATOM | 1707 | CB | LEU | A | 221 | −9.180 | −3.602 | −8.599 | 1.00 | 20.48 |
| ATOM | 1708 | CG | LEU | A | 221 | −10.336 | −2.991 | −7.790 | 1.00 | 22.48 |
| ATOM | 1709 | CD1 | LEU | A | 221 | −10.857 | −1.726 | −8.458 | 1.00 | 22.33 |
| ATOM | 1710 | CD2 | LEU | A | 221 | −11.430 | −4.011 | −7.637 | 1.00 | 22.51 |
| ATOM | 1711 | C | LEU | A | 221 | −7.360 | −2.591 | −7.192 | 1.00 | 21.44 |
| ATOM | 1712 | O | LEU | A | 221 | −7.617 | −1.578 | −6.539 | 1.00 | 20.45 |
| ATOM | 1713 | N | CYS | A | 222 | −6.600 | −3.572 | −6.718 | 1.00 | 22.60 |
| ATOM | 1714 | CA | CYS | A | 222 | −5.957 | −3.477 | −5.415 | 1.00 | 22.10 |
| ATOM | 1715 | CB | CYS | A | 222 | −5.159 | −4.749 | −5.125 | 1.00 | 23.41 |
| ATOM | 1716 | SG | CYS | A | 222 | −4.975 | −5.000 | −3.356 | 1.00 | 28.49 |
| ATOM | 1717 | C | CYS | A | 222 | −5.035 | −2.270 | −5.317 | 1.00 | 21.22 |
| ATOM | 1718 | O | CYS | A | 222 | −5.060 | −1.531 | −4.331 | 1.00 | 21.24 |
| ATOM | 1719 | N | PHE | A | 223 | −4.210 | −2.070 | −6.347 | 1.00 | 20.11 |
| ATOM | 1720 | CA | PHE | A | 223 | −3.287 | −0.955 | −6.368 | 1.00 | 19.03 |
| ATOM | 1721 | CB | PHE | A | 223 | −2.334 | −1.108 | −7.558 | 1.00 | 19.15 |
| ATOM | 1722 | CG | PHE | A | 223 | −1.297 | −0.011 | −7.669 | 1.00 | 19.23 |
| ATOM | 1723 | CD1 | PHE | A | 223 | −0.576 | 0.410 | −6.558 | 1.00 | 19.90 |
| ATOM | 1724 | CE1 | PHE | A | 223 | 0.380 | 1.417 | −6.661 | 1.00 | 20.91 |
| ATOM | 1725 | CZ | PHE | A | 223 | 0.645 | 2.017 | −7.902 | 1.00 | 21.07 |
| ATOM | 1726 | CE2 | PHE | A | 223 | −0.061 | 1.598 | −9.024 | 1.00 | 18.81 |
| ATOM | 1727 | CD2 | PHE | A | 223 | −1.022 | 0.581 | −8.909 | 1.00 | 18.23 |
| ATOM | 1728 | C | PHE | A | 223 | −4.032 | 0.397 | −6.423 | 1.00 | 18.38 |
| ATOM | 1729 | O | PHE | A | 223 | −3.597 | 1.376 | −5.818 | 1.00 | 18.27 |
| ATOM | 1730 | N | LEU | A | 224 | −5.148 | 0.428 | −7.142 | 1.00 | 18.45 |
| ATOM | 1731 | CA | LEU | A | 224 | −5.957 | 1.665 | −7.277 | 1.00 | 18.42 |
| ATOM | 1732 | CB | LEU | A | 224 | −7.208 | 1.403 | −8.127 | 1.00 | 17.70 |
| ATOM | 1733 | CG | LEU | A | 224 | −7.990 | 2.645 | −8.610 | 1.00 | 19.73 |
| ATOM | 1734 | CD1 | LEU | A | 224 | −7.133 | 3.427 | −9.584 | 1.00 | 20.37 |
| ATOM | 1735 | CD2 | LEU | A | 224 | −9.302 | 2.228 | −9.264 | 1.00 | 18.64 |
| ATOM | 1736 | C | LEU | A | 224 | −6.385 | 2.226 | −5.917 | 1.00 | 18.87 |
| ATOM | 1737 | O | LEU | A | 224 | −6.553 | 3.438 | −5.757 | 1.00 | 18.45 |
| ATOM | 1738 | N | GLN | A | 225 | −6.578 | 1.336 | −4.944 | 1.00 | 19.17 |
| ATOM | 1739 | CA | GLN | A | 225 | −6.984 | 1.743 | −3.585 | 1.00 | 20.00 |
| ATOM | 1740 | CB | GLN | A | 225 | −7.340 | 0.511 | −2.725 | 1.00 | 20.26 |
| ATOM | 1741 | CG | GLN | A | 225 | −8.295 | −0.463 | −3.409 | 1.00 | 21.22 |
| ATOM | 1742 | CD | GLN | A | 225 | −9.519 | 0.225 | −3.993 | 1.00 | 22.53 |
| ATOM | 1743 | OE1 | GLN | A | 225 | −10.280 | 0.870 | −3.262 | 1.00 | 23.09 |
| ATOM | 1744 | NE2 | GLN | A | 225 | −9.718 | 0.092 | −5.302 | 1.00 | 19.33 |
| ATOM | 1745 | C | GLN | A | 225 | −5.944 | 2.599 | −2.871 | 1.00 | 20.19 |
| ATOM | 1746 | O | GLN | A | 225 | −6.299 | 3.399 | −2.009 | 1.00 | 20.64 |
| ATOM | 1747 | N | ARG | A | 226 | −4.678 | 2.450 | −3.253 | 1.00 | 20.51 |
| ATOM | 1748 | CA | ARG | A | 226 | −3.564 | 3.144 | −2.608 | 1.00 | 21.40 |
| ATOM | 1749 | CB | ARG | A | 226 | −2.219 | 2.505 | −2.990 | 1.00 | 22.72 |
| ATOM | 1750 | CG | ARG | A | 226 | −2.081 | 1.010 | −2.683 | 1.00 | 26.14 |
| ATOM | 1751 | CD | ARG | A | 226 | −1.806 | 0.741 | −1.204 | 1.00 | 32.16 |
| ATOM | 1752 | NE | ARG | A | 226 | −3.035 | 0.843 | −0.432 | 1.00 | 37.77 |
| ATOM | 1753 | CZ | ARG | A | 226 | −3.997 | −0.079 | −0.413 | 1.00 | 41.09 |
| ATOM | 1754 | NH1 | ARG | A | 226 | −5.093 | 0.120 | 0.322 | 1.00 | 42.17 |
| ATOM | 1755 | NH2 | ARG | A | 226 | −3.874 | −1.196 | −1.127 | 1.00 | 42.78 |
| ATOM | 1756 | C | ARG | A | 226 | −3.499 | 4.645 | −2.915 | 1.00 | 21.23 |
| ATOM | 1757 | O | ARG | A | 226 | −2.723 | 5.358 | −2.288 | 1.00 | 20.95 |
| ATOM | 1758 | N | PHE | A | 227 | −4.298 | 5.123 | −3.869 | 1.00 | 20.28 |
| ATOM | 1759 | CA | PHE | A | 227 | −4.280 | 6.545 | −4.250 | 1.00 | 19.67 |
| ATOM | 1760 | CB | PHE | A | 227 | −4.777 | 6.704 | −5.693 | 1.00 | 19.40 |
| ATOM | 1761 | CG | PHE | A | 227 | −3.814 | 6.195 | −6.744 | 1.00 | 18.28 |
| ATOM | 1762 | CD1 | PHE | A | 227 | −3.733 | 4.831 | −7.040 | 1.00 | 18.14 |
| ATOM | 1763 | CE1 | PHE | A | 227 | −2.855 | 4.355 | −8.046 | 1.00 | 18.24 |
| ATOM | 1764 | CZ | PHE | A | 227 | −2.034 | 5.264 | −8.748 | 1.00 | 16.75 |
| ATOM | 1765 | CE2 | PHE | A | 227 | −2.113 | 6.641 | −8.456 | 1.00 | 18.79 |
| ATOM | 1766 | CD2 | PHE | A | 227 | −3.005 | 7.091 | −7.452 | 1.00 | 17.51 |
| ATOM | 1767 | C | PHE | A | 227 | −5.126 | 7.435 | −3.343 | 1.00 | 20.55 |
| ATOM | 1768 | O | PHE | A | 227 | −4.967 | 8.659 | −3.334 | 1.00 | 20.38 |
| ATOM | 1769 | N | TRP | A | 228 | −6.032 | 6.820 | −2.583 | 1.00 | 20.72 |
| ATOM | 1770 | CA | TRP | A | 228 | −6.924 | 7.545 | −1.671 | 1.00 | 20.71 |
| ATOM | 1771 | CB | TRP | A | 228 | −8.036 | 6.596 | −1.211 | 1.00 | 20.41 |
| ATOM | 1772 | CG | TRP | A | 228 | −9.030 | 7.228 | −0.283 | 1.00 | 20.59 |
| ATOM | 1773 | CD1 | TRP | A | 228 | −9.243 | 6.915 | 1.040 | 1.00 | 21.81 |
| ATOM | 1774 | NE1 | TRP | A | 228 | −10.255 | 7.722 | 1.557 | 1.00 | 22.69 |
| ATOM | 1775 | CE2 | TRP | A | 228 | −10.712 | 8.553 | 0.565 | 1.00 | 20.71 |
| ATOM | 1776 | CD2 | TRP | A | 228 | −9.958 | 8.280 | −0.607 | 1.00 | 18.88 |
| ATOM | 1777 | CE3 | TRP | A | 228 | −10.225 | 9.014 | −1.772 | 1.00 | 18.79 |
| ATOM | 1778 | CZ3 | TRP | A | 228 | −11.209 | 9.986 | −1.734 | 1.00 | 20.13 |
| ATOM | 1779 | CH2 | TRP | A | 228 | −11.937 | 10.242 | −0.552 | 1.00 | 21.18 |
| ATOM | 1780 | CZ2 | TRP | A | 228 | −11.710 | 9.537 | 0.601 | 1.00 | 21.65 |
| ATOM | 1781 | C | TRP | A | 228 | −6.193 | 8.120 | −0.463 | 1.00 | 21.38 |
| ATOM | 1782 | O | TRP | A | 228 | −5.479 | 7.394 | 0.236 | 1.00 | 21.50 |
| ATOM | 1783 | N | VAL | A | 229 | −6.379 | 9.416 | −0.209 | 1.00 | 21.95 |
| ATOM | 1784 | CA | VAL | A | 229 | −5.844 | 10.065 | 0.983 | 1.00 | 22.99 |
| ATOM | 1785 | CB | VAL | A | 229 | −5.205 | 11.436 | 0.654 | 1.00 | 22.81 |
| ATOM | 1786 | CG1 | VAL | A | 229 | −4.490 | 12.026 | 1.871 | 1.00 | 23.48 |
| ATOM | 1787 | CG2 | VAL | A | 229 | −4.226 | 11.292 | −0.493 | 1.00 | 23.28 |
| ATOM | 1788 | C | VAL | A | 229 | −6.984 | 10.206 | 2.000 | 1.00 | 24.08 |
| ATOM | 1789 | O | VAL | A | 229 | −7.803 | 11.119 | 1.899 | 1.00 | 23.70 |
| ATOM | 1790 | N | SER | A | 230 | −7.044 | 9.298 | 2.974 | 1.00 | 25.37 |
| ATOM | 1791 | CA | SER | A | 230 | −8.193 | 9.290 | 3.905 | 1.00 | 27.59 |
| ATOM | 1792 | CB | SER | A | 230 | −8.254 | 8.000 | 4.728 | 1.00 | 27.67 |
| ATOM | 1793 | OG | SER | A | 230 | −7.029 | 7.805 | 5.402 | 1.00 | 31.30 |
| ATOM | 1794 | C | SER | A | 230 | −8.241 | 10.513 | 4.820 | 1.00 | 27.93 |
| ATOM | 1795 | O | SER | A | 230 | −9.321 | 10.983 | 5.174 | 1.00 | 28.91 |
| ATOM | 1796 | N | SER | A | 231 | −7.088 | 11.059 | 5.165 | 1.00 | 28.76 |
| ATOM | 1797 | CA | SER | A | 231 | −7.059 | 12.237 | 6.030 | 1.00 | 29.72 |
| ATOM | 1798 | CB | SER | A | 231 | −5.671 | 12.461 | 6.639 | 1.00 | 30.39 |
| ATOM | 1799 | OG | SER | A | 231 | −4.703 | 12.713 | 5.635 | 1.00 | 34.43 |
| ATOM | 1800 | C | SER | A | 231 | −7.566 | 13.491 | 5.323 | 1.00 | 29.39 |
| ATOM | 1801 | O | SER | A | 231 | −8.154 | 14.364 | 5.966 | 1.00 | 30.97 |
| ATOM | 1802 | N | GLY | A | 232 | −7.373 | 13.579 | 4.005 | 1.00 | 27.59 |
| ATOM | 1803 | CA | GLY | A | 232 | −7.867 | 14.728 | 3.247 | 1.00 | 25.22 |
| ATOM | 1804 | C | GLY | A | 232 | −9.181 | 14.518 | 2.493 | 1.00 | 23.25 |
| ATOM | 1805 | O | GLY | A | 232 | −9.810 | 15.487 | 2.077 | 1.00 | 23.19 |
| ATOM | 1806 | N | GLY | A | 233 | −9.589 | 13.265 | 2.320 | 1.00 | 20.97 |
| ATOM | 1807 | CA | GLY | A | 233 | −10.809 | 12.937 | 1.578 | 1.00 | 19.35 |
| ATOM | 1808 | C | GLY | A | 233 | −10.673 | 13.226 | 0.094 | 1.00 | 18.83 |
| ATOM | 1809 | O | GLY | A | 233 | −11.636 | 13.655 | −0.561 | 1.00 | 19.20 |
| ATOM | 1810 | N | TYR | A | 234 | −9.487 | 12.977 | −0.463 | 1.00 | 17.56 |
| ATOM | 1811 | CA | TYR | A | 234 | −9.309 | 13.155 | −1.915 | 1.00 | 17.17 |
| ATOM | 1812 | CB | TYR | A | 234 | −8.851 | 14.584 | −2.232 | 1.00 | 18.33 |
| ATOM | 1813 | CG | TYR | A | 234 | −7.441 | 14.876 | −1.758 | 1.00 | 20.39 |
| ATOM | 1814 | CD1 | TYR | A | 234 | −7.203 | 15.340 | −0.454 | 1.00 | 20.72 |
| ATOM | 1815 | CE1 | TYR | A | 234 | −5.905 | 15.594 | −0.018 | 1.00 | 24.11 |
| ATOM | 1816 | CZ | TYR | A | 234 | −4.840 | 15.399 | −0.897 | 1.00 | 23.78 |
| ATOM | 1817 | OH | TYR | A | 234 | −3.556 | 15.663 | −0.483 | 1.00 | 26.50 |
| ATOM | 1818 | CE2 | TYR | A | 234 | −5.055 | 14.956 | −2.187 | 1.00 | 24.07 |
| ATOM | 1819 | CD2 | TYR | A | 234 | −6.353 | 14.699 | −2.611 | 1.00 | 20.58 |
| ATOM | 1820 | C | TYR | A | 234 | −8.318 | 12.141 | −2.482 | 1.00 | 16.60 |
| ATOM | 1821 | O | TYR | A | 234 | −7.615 | 11.465 | −1.735 | 1.00 | 16.29 |
| ATOM | 1822 | N | VAL | A | 235 | −8.260 | 12.059 | −3.805 | 1.00 | 15.36 |
| ATOM | 1823 | CA | VAL | A | 235 | −7.325 | 11.164 | −4.472 | 1.00 | 15.62 |
| ATOM | 1824 | CB | VAL | A | 235 | −7.948 | 10.638 | −5.798 | 1.00 | 15.96 |
| ATOM | 1825 | CG1 | VAL | A | 235 | −6.889 | 9.893 | −6.645 | 1.00 | 17.31 |
| ATOM | 1826 | CG2 | VAL | A | 235 | −9.134 | 9.723 | −5.506 | 1.00 | 15.87 |
| ATOM | 1827 | C | VAL | A | 235 | −6.011 | 11.904 | −4.742 | 1.00 | 15.54 |
| ATOM | 1828 | O | VAL | A | 235 | −6.006 | 12.998 | −5.320 | 1.00 | 15.39 |
| ATOM | 1829 | N | ASP | A | 236 | −4.886 | 11.316 | −4.325 | 1.00 | 15.24 |
| ATOM | 1830 | CA | ASP | A | 236 | −3.580 | 11.837 | −4.705 | 1.00 | 15.22 |
| ATOM | 1831 | CB | ASP | A | 236 | −2.533 | 11.431 | −3.652 | 1.00 | 16.45 |
| ATOM | 1832 | CG | ASP | A | 236 | −1.145 | 11.922 | −3.970 | 1.00 | 18.62 |
| ATOM | 1833 | OD1 | ASP | A | 236 | −0.937 | 12.617 | −4.992 | 1.00 | 17.17 |
| ATOM | 1834 | OD2 | ASP | A | 236 | −0.223 | 11.568 | −3.182 | 1.00 | 22.79 |
| ATOM | 1835 | C | ASP | A | 236 | −3.303 | 11.256 | −6.098 | 1.00 | 15.06 |
| ATOM | 1836 | O | ASP | A | 236 | −3.088 | 10.040 | −6.261 | 1.00 | 15.39 |
| ATOM | 1837 | N | SER | A | 237 | −3.384 | 12.104 | −7.125 | 1.00 | 14.24 |
| ATOM | 1838 | CA | SER | A | 237 | −3.518 | 11.587 | −8.503 | 1.00 | 14.09 |
| ATOM | 1839 | CB | SER | A | 237 | −4.000 | 12.697 | −9.446 | 1.00 | 13.76 |
| ATOM | 1840 | OG | SER | A | 237 | −5.312 | 13.094 | −9.070 | 1.00 | 14.52 |
| ATOM | 1841 | C | SER | A | 237 | −2.277 | 10.883 | −9.053 | 1.00 | 14.22 |
| ATOM | 1842 | O | SER | A | 237 | −2.376 | 10.067 | −9.965 | 1.00 | 13.80 |
| ATOM | 1843 | N | ASN | A | 238 | −1.099 | 11.219 | −8.521 | 1.00 | 14.70 |
| ATOM | 1844 | CA | ASN | A | 238 | 0.116 | 10.547 | −8.952 | 1.00 | 15.28 |
| ATOM | 1845 | CB | ASN | A | 238 | 0.968 | 11.439 | −9.856 | 1.00 | 14.84 |
| ATOM | 1846 | CG | ASN | A | 238 | 0.277 | 11.742 | −11.176 | 1.00 | 17.08 |
| ATOM | 1847 | OD1 | ASN | A | 238 | 0.244 | 10.901 | −12.072 | 1.00 | 16.61 |
| ATOM | 1848 | ND2 | ASN | A | 238 | −0.308 | 12.932 | −11.278 | 1.00 | 16.63 |
| ATOM | 1849 | C | ASN | A | 238 | 0.912 | 10.150 | −7.736 | 1.00 | 16.07 |
| ATOM | 1850 | O | ASN | A | 238 | 1.169 | 10.988 | −6.890 | 1.00 | 15.88 |
| ATOM | 1851 | N | ILE | A | 239 | 1.280 | 8.875 | −7.659 | 1.00 | 16.09 |
| ATOM | 1852 | CA | ILE | A | 239 | 2.125 | 8.410 | −6.567 | 1.00 | 18.07 |
| ATOM | 1853 | CB | ILE | A | 239 | 1.340 | 7.452 | −5.600 | 1.00 | 17.66 |
| ATOM | 1854 | CG1 | ILE | A | 239 | 0.893 | 6.180 | −6.336 | 1.00 | 18.85 |
| ATOM | 1855 | CD1 | ILE | A | 239 | 0.184 | 5.109 | −5.437 | 1.00 | 19.02 |
| ATOM | 1856 | CG2 | ILE | A | 239 | 0.116 | 8.194 | −4.974 | 1.00 | 16.96 |
| ATOM | 1857 | C | ILE | A | 239 | 3.381 | 7.760 | −7.169 | 1.00 | 19.32 |
| ATOM | 1858 | O | ILE | A | 239 | 3.571 | 7.797 | −8.392 | 1.00 | 19.19 |
| ATOM | 1859 | N | ASN | A | 240 | 4.242 | 7.170 | −6.329 | 1.00 | 20.56 |
| ATOM | 1860 | CA | ASN | A | 240 | 5.517 | 6.617 | −6.823 | 1.00 | 22.24 |
| ATOM | 1861 | CB | ASN | A | 240 | 5.275 | 5.385 | −7.717 | 1.00 | 21.93 |
| ATOM | 1862 | CG | ASN | A | 240 | 4.874 | 4.153 | −6.926 | 1.00 | 24.19 |
| ATOM | 1863 | OD1 | ASN | A | 240 | 5.269 | 3.995 | −5.772 | 1.00 | 25.98 |
| ATOM | 1864 | ND2 | ASN | A | 240 | 4.083 | 3.278 | −7.538 | 1.00 | 22.26 |
| ATOM | 1865 | C | ASN | A | 240 | 6.334 | 7.677 | −7.571 | 1.00 | 23.27 |
| ATOM | 1866 | O | ASN | A | 240 | 7.000 | 7.381 | −8.562 | 1.00 | 23.15 |
| ATOM | 1867 | N | THR | A | 241 | 6.261 | 8.919 | −7.096 | 1.00 | 25.02 |
| ATOM | 1868 | CA | THR | A | 241 | 6.939 | 10.038 | −7.729 | 1.00 | 28.12 |
| ATOM | 1869 | CB | THR | A | 241 | 6.044 | 10.720 | −8.817 | 1.00 | 28.09 |
| ATOM | 1870 | OG1 | THR | A | 241 | 6.741 | 11.836 | −9.369 | 1.00 | 28.75 |
| ATOM | 1871 | CG2 | THR | A | 241 | 4.727 | 11.208 | −8.231 | 1.00 | 28.30 |
| ATOM | 1872 | C | THR | A | 241 | 7.302 | 11.065 | −6.674 | 1.00 | 29.96 |
| ATOM | 1873 | O | THR | A | 241 | 6.749 | 11.037 | −5.589 | 1.00 | 30.58 |
| ATOM | 1874 | N | ASN | A | 242 | 8.209 | 11.984 | −6.991 | 1.00 | 33.17 |
| ATOM | 1875 | CA | ASN | A | 242 | 8.585 | 13.019 | −6.024 | 1.00 | 36.07 |
| ATOM | 1876 | CB | ASN | A | 242 | 10.059 | 12.880 | −5.616 | 1.00 | 37.13 |
| ATOM | 1877 | CG | ASN | A | 242 | 10.324 | 11.631 | −4.771 | 1.00 | 40.96 |
| ATOM | 1878 | OD1 | ASN | A | 242 | 9.509 | 11.235 | −3.921 | 1.00 | 45.33 |
| ATOM | 1879 | ND2 | ASN | A | 242 | 11.477 | 11.007 | −4.998 | 1.00 | 44.43 |
| ATOM | 1880 | C | ASN | A | 242 | 8.321 | 14.427 | −6.528 | 1.00 | 37.00 |
| ATOM | 1881 | O | ASN | A | 242 | 9.091 | 15.346 | −6.245 | 1.00 | 37.94 |
| ATOM | 1882 | N | GLU | A | 243 | 7.210 | 14.602 | −7.233 | 1.00 | 37.54 |
| ATOM | 1883 | CA | GLU | A | 243 | 6.895 | 15.869 | −7.907 | 1.00 | 38.05 |
| ATOM | 1884 | CB | GLU | A | 243 | 5.775 | 15.638 | −8.925 | 1.00 | 38.77 |
| ATOM | 1885 | CG | GLU | A | 243 | 5.650 | 16.732 | −9.977 | 1.00 | 42.65 |
| ATOM | 1886 | CD | GLU | A | 243 | 6.959 | 16.985 | −10.709 | 1.00 | 47.49 |
| ATOM | 1887 | OE1 | GLU | A | 243 | 7.424 | 16.084 | −11.453 | 1.00 | 49.14 |
| ATOM | 1888 | OE2 | GLU | A | 243 | 7.520 | 18.090 | −10.532 | 1.00 | 50.15 |
| ATOM | 1889 | C | GLU | A | 243 | 6.559 | 17.088 | −7.015 | 1.00 | 37.15 |
| ATOM | 1890 | O | GLU | A | 243 | 6.645 | 18.240 | −7.469 | 1.00 | 38.39 |
| ATOM | 1891 | N | GLY | A | 244 | 6.174 | 16.873 | −5.766 | 1.00 | 35.69 |
| ATOM | 1892 | CA | GLY | A | 244 | 5.858 | 18.019 | −4.911 | 1.00 | 33.51 |
| ATOM | 1893 | C | GLY | A | 244 | 4.609 | 18.775 | −5.369 | 1.00 | 31.80 |
| ATOM | 1894 | O | GLY | A | 244 | 4.634 | 19.999 | −5.535 | 1.00 | 33.32 |
| ATOM | 1895 | N | ARG | A | 245 | 3.529 | 18.036 | −5.612 | 1.00 | 27.92 |
| ATOM | 1896 | CA | ARG | A | 245 | 2.200 | 18.618 | −5.781 | 1.00 | 24.21 |
| ATOM | 1897 | CB | ARG | A | 245 | 1.638 | 18.224 | −7.130 | 1.00 | 24.30 |
| ATOM | 1898 | CG | ARG | A | 245 | 2.410 | 18.842 | −8.275 | 1.00 | 24.62 |
| ATOM | 1899 | CD | ARG | A | 245 | 1.625 | 18.681 | −9.532 | 1.00 | 22.11 |
| ATOM | 1900 | NE | ARG | A | 245 | 2.462 | 18.829 | −10.713 | 1.00 | 21.13 |
| ATOM | 1901 | CZ | ARG | A | 245 | 2.114 | 18.302 | −11.878 | 1.00 | 21.50 |
| ATOM | 1902 | NH1 | ARG | A | 245 | 0.982 | 17.621 | −11.945 | 1.00 | 18.57 |
| ATOM | 1903 | NH2 | ARG | A | 245 | 2.883 | 18.443 | −12.951 | 1.00 | 20.83 |
| ATOM | 1904 | C | ARG | A | 245 | 1.295 | 18.040 | −4.718 | 1.00 | 21.84 |
| ATOM | 1905 | O | ARG | A | 245 | 1.624 | 17.021 | −4.128 | 1.00 | 20.65 |
| ATOM | 1906 | N | THR | A | 246 | 0.140 | 18.652 | −4.483 | 1.00 | 19.15 |
| ATOM | 1907 | CA | THR | A | 246 | −0.824 | 18.058 | −3.540 | 1.00 | 17.46 |
| ATOM | 1908 | CB | THR | A | 246 | −1.989 | 18.997 | −3.238 | 1.00 | 17.87 |
| ATOM | 1909 | OG1 | THR | A | 246 | −2.752 | 19.155 | −4.440 | 1.00 | 15.85 |
| ATOM | 1910 | CG2 | THR | A | 246 | −1.495 | 20.370 | −2.730 | 1.00 | 17.50 |
| ATOM | 1911 | C | THR | A | 246 | −1.426 | 16.769 | −4.103 | 1.00 | 17.25 |
| ATOM | 1912 | O | THR | A | 246 | −1.884 | 15.914 | −3.351 | 1.00 | 17.57 |
| ATOM | 1913 | N | GLY | A | 247 | −1.482 | 16.646 | −5.430 | 1.00 | 15.60 |
| ATOM | 1914 | CA | GLY | A | 247 | −2.148 | 15.492 | −6.054 | 1.00 | 15.02 |
| ATOM | 1915 | C | GLY | A | 247 | −3.609 | 15.761 | −6.396 | 1.00 | 14.69 |
| ATOM | 1916 | O | GLY | A | 247 | −4.260 | 14.939 | −7.059 | 1.00 | 14.45 |
| ATOM | 1917 | N | LYS | A | 248 | −4.137 | 16.890 | −5.928 | 1.00 | 13.43 |
| ATOM | 1918 | CA | LYS | A | 248 | −5.508 | 17.286 | −6.259 | 1.00 | 13.00 |
| ATOM | 1919 | CB | LYS | A | 248 | −5.969 | 18.453 | −5.396 | 1.00 | 12.32 |
| ATOM | 1920 | CG | LYS | A | 248 | −5.965 | 18.179 | −3.881 | 1.00 | 13.12 |
| ATOM | 1921 | CD | LYS | A | 248 | −6.133 | 19.493 | −3.102 | 1.00 | 14.08 |
| ATOM | 1922 | CE | LYS | A | 248 | −5.985 | 19.253 | −1.584 | 1.00 | 17.84 |
| ATOM | 1923 | NZ | LYS | A | 248 | −6.335 | 20.492 | −0.835 | 1.00 | 16.74 |
| ATOM | 1924 | C | LYS | A | 248 | −5.490 | 17.713 | −7.736 | 1.00 | 12.73 |
| ATOM | 1925 | O | LYS | A | 248 | −4.866 | 18.707 | −8.104 | 1.00 | 12.75 |
| ATOM | 1926 | N | ASP | A | 249 | −6.185 | 16.964 | −8.580 | 1.00 | 11.92 |
| ATOM | 1927 | CA | ASP | A | 249 | −5.958 | 17.098 | −10.024 | 1.00 | 11.16 |
| ATOM | 1928 | CB | ASP | A | 249 | −4.761 | 16.199 | −10.385 | 1.00 | 10.83 |
| ATOM | 1929 | CG | ASP | A | 249 | −4.268 | 16.349 | −11.831 | 1.00 | 12.54 |
| ATOM | 1930 | OD1 | ASP | A | 249 | −5.078 | 16.422 | −12.785 | 1.00 | 11.42 |
| ATOM | 1931 | OD2 | ASP | A | 249 | −3.025 | 16.342 | −12.001 | 1.00 | 13.30 |
| ATOM | 1932 | C | ASP | A | 249 | −7.232 | 16.577 | −10.662 | 1.00 | 11.38 |
| ATOM | 1933 | O | ASP | A | 249 | −7.774 | 15.542 | −10.236 | 1.00 | 10.86 |
| ATOM | 1934 | N | VAL | A | 250 | −7.700 | 17.265 | −11.703 | 1.00 | 11.28 |
| ATOM | 1935 | CA | VAL | A | 250 | −8.885 | 16.793 | −12.438 | 1.00 | 11.59 |
| ATOM | 1936 | CB | VAL | A | 250 | −9.366 | 17.859 | −13.493 | 1.00 | 12.49 |
| ATOM | 1937 | CG1 | VAL | A | 250 | −8.480 | 17.815 | −14.728 | 1.00 | 13.03 |
| ATOM | 1938 | CG2 | VAL | A | 250 | −10.859 | 17.654 | −13.852 | 1.00 | 13.75 |
| ATOM | 1939 | C | VAL | A | 250 | −8.711 | 15.386 | −13.064 | 1.00 | 11.77 |
| ATOM | 1940 | O | VAL | A | 250 | −9.698 | 14.750 | −13.467 | 1.00 | 11.71 |
| ATOM | 1941 | N | ASN | A | 251 | −7.461 | 14.925 | −13.168 | 1.00 | 10.73 |
| ATOM | 1942 | CA | ASN | A | 251 | −7.131 | 13.491 | −13.378 | 1.00 | 11.20 |
| ATOM | 1943 | CB | ASN | A | 251 | −5.699 | 13.265 | −12.813 | 1.00 | 10.94 |
| ATOM | 1944 | CG | ASN | A | 251 | −5.221 | 11.810 | −12.892 | 1.00 | 11.58 |
| ATOM | 1945 | OD1 | ASN | A | 251 | −5.986 | 10.864 | −12.672 | 1.00 | 12.47 |
| ATOM | 1946 | ND2 | ASN | A | 251 | −3.898 | 11.639 | −13.164 | 1.00 | 14.40 |
| ATOM | 1947 | C | ASN | A | 251 | −8.151 | 12.560 | −12.706 | 1.00 | 10.99 |
| ATOM | 1948 | O | ASN | A | 251 | −8.755 | 11.706 | −13.355 | 1.00 | 11.49 |
| ATOM | 1949 | N | SER | A | 252 | −8.407 | 12.774 | −11.417 | 1.00 | 11.45 |
| ATOM | 1950 | CA | SER | A | 252 | −9.293 | 11.876 | −10.634 | 1.00 | 11.79 |
| ATOM | 1951 | CB | SER | A | 252 | −9.062 | 12.155 | −9.149 | 1.00 | 13.31 |
| ATOM | 1952 | OG | SER | A | 252 | −9.338 | 13.524 | −8.882 | 1.00 | 13.41 |
| ATOM | 1953 | C | SER | A | 252 | −10.784 | 12.002 | −10.996 | 1.00 | 11.39 |
| ATOM | 1954 | O | SER | A | 252 | −11.532 | 11.023 | −10.964 | 1.00 | 12.69 |
| ATOM | 1955 | N | VAL | A | 253 | −11.199 | 13.203 | −11.383 | 1.00 | 10.56 |
| ATOM | 1956 | CA | VAL | A | 253 | −12.582 | 13.459 | −11.821 | 1.00 | 10.70 |
| ATOM | 1957 | CB | VAL | A | 253 | −12.884 | 15.004 | −11.856 | 1.00 | 11.02 |
| ATOM | 1958 | CG1 | VAL | A | 253 | −14.335 | 15.262 | −12.345 | 1.00 | 11.24 |
| ATOM | 1959 | CG2 | VAL | A | 253 | −12.711 | 15.585 | −10.449 | 1.00 | 10.91 |
| ATOM | 1960 | C | VAL | A | 253 | −12.810 | 12.827 | −13.187 | 1.00 | 11.38 |
| ATOM | 1961 | O | VAL | A | 253 | −13.824 | 12.143 | −13.407 | 1.00 | 11.69 |
| ATOM | 1962 | N | LEU | A | 254 | −11.866 | 13.059 | −14.108 | 1.00 | 11.32 |
| ATOM | 1963 | CA | LEU | A | 254 | −11.891 | 12.393 | −15.417 | 1.00 | 12.12 |
| ATOM | 1964 | CB | LEU | A | 254 | −10.635 | 12.759 | −16.238 | 1.00 | 11.95 |
| ATOM | 1965 | CG | LEU | A | 254 | −10.634 | 14.202 | −16.763 | 1.00 | 12.23 |
| ATOM | 1966 | CD1 | LEU | A | 254 | −9.266 | 14.564 | −17.330 | 1.00 | 12.77 |
| ATOM | 1967 | CD2 | LEU | A | 254 | −11.714 | 14.371 | −17.845 | 1.00 | 15.26 |
| ATOM | 1968 | C | LEU | A | 254 | −11.963 | 10.872 | −15.271 | 1.00 | 12.22 |
| ATOM | 1969 | O | LEU | A | 254 | −12.675 | 10.201 | −16.024 | 1.00 | 12.01 |
| ATOM | 1970 | N | THR | A | 255 | −11.208 | 10.338 | −14.315 | 1.00 | 11.58 |
| ATOM | 1971 | CA | THR | A | 255 | −11.219 | 8.913 | −14.042 | 1.00 | 12.53 |
| ATOM | 1972 | CB | THR | A | 255 | −10.267 | 8.552 | −12.890 | 1.00 | 12.83 |
| ATOM | 1973 | OG1 | THR | A | 255 | −8.935 | 8.933 | −13.240 | 1.00 | 13.00 |
| ATOM | 1974 | CG2 | THR | A | 255 | −10.300 | 7.035 | −12.634 | 1.00 | 15.06 |
| ATOM | 1975 | C | THR | A | 255 | −12.632 | 8.448 | −13.705 | 1.00 | 13.12 |
| ATOM | 1976 | O | THR | A | 255 | −13.131 | 7.467 | −14.285 | 1.00 | 13.49 |
| ATOM | 1977 | N | SER | A | 256 | −13.289 | 9.158 | −12.790 | 1.00 | 13.46 |
| ATOM | 1978 | CA | SER | A | 256 | −14.641 | 8.781 | −12.343 | 1.00 | 12.85 |
| ATOM | 1979 | CB | SER | A | 256 | −15.152 | 9.760 | −11.282 | 1.00 | 13.40 |
| ATOM | 1980 | OG | SER | A | 256 | −16.332 | 9.252 | −10.674 | 1.00 | 16.69 |
| ATOM | 1981 | C | SER | A | 256 | −15.610 | 8.705 | −13.518 | 1.00 | 13.13 |
| ATOM | 1982 | O | SER | A | 256 | −16.360 | 7.711 | −13.654 | 1.00 | 13.10 |
| ATOM | 1983 | N | ILE | A | 257 | −15.594 | 9.728 | −14.377 | 1.00 | 12.32 |
| ATOM | 1984 | CA | ILE | A | 257 | −16.523 | 9.784 | −15.513 | 1.00 | 12.32 |
| ATOM | 1985 | CB | ILE | A | 257 | −16.747 | 11.215 | −16.072 | 1.00 | 11.55 |
| ATOM | 1986 | CG1 | ILE | A | 257 | −15.482 | 11.773 | −16.764 | 1.00 | 11.38 |
| ATOM | 1987 | CD1 | ILE | A | 257 | −15.699 | 13.143 | −17.441 | 1.00 | 13.23 |
| ATOM | 1988 | CG2 | ILE | A | 257 | −17.257 | 12.166 | −14.942 | 1.00 | 13.70 |
| ATOM | 1989 | C | ILE | A | 257 | −16.220 | 8.795 | −16.653 | 1.00 | 12.79 |
| ATOM | 1990 | O | ILE | A | 257 | −17.150 | 8.319 | −17.338 | 1.00 | 13.25 |
| ATOM | 1991 | N | HIS | A | 258 | −14.941 | 8.487 | −16.855 | 1.00 | 12.71 |
| ATOM | 1992 | CA | HIS | A | 258 | −14.565 | 7.566 | −17.931 | 1.00 | 13.41 |
| ATOM | 1993 | CB | HIS | A | 258 | −13.194 | 7.947 | −18.498 | 1.00 | 12.06 |
| ATOM | 1994 | CG | HIS | A | 258 | −13.268 | 9.175 | −19.341 | 1.00 | 13.92 |
| ATOM | 1995 | ND1 | HIS | A | 258 | −13.942 | 9.196 | −20.547 | 1.00 | 16.01 |
| ATOM | 1996 | CE1 | HIS | A | 258 | −13.891 | 10.421 | −21.047 | 1.00 | 18.57 |
| ATOM | 1997 | NE2 | HIS | A | 258 | −13.256 | 11.199 | −20.189 | 1.00 | 14.08 |
| ATOM | 1998 | CD2 | HIS | A | 258 | −12.861 | 10.449 | −19.108 | 1.00 | 13.51 |
| ATOM | 1999 | C | HIS | A | 258 | −14.649 | 6.091 | −17.565 | 1.00 | 14.12 |
| ATOM | 2000 | O | HIS | A | 258 | −14.645 | 5.239 | −18.454 | 1.00 | 14.90 |
| ATOM | 2001 | N | THR | A | 259 | −14.752 | 5.801 | −16.274 | 1.00 | 13.93 |
| ATOM | 2002 | CA | THR | A | 259 | −15.034 | 4.420 | −15.807 | 1.00 | 14.91 |
| ATOM | 2003 | CB | THR | A | 259 | −13.933 | 3.856 | −14.899 | 1.00 | 14.46 |
| ATOM | 2004 | OG1 | THR | A | 259 | −13.788 | 4.647 | −13.705 | 1.00 | 15.66 |
| ATOM | 2005 | CG2 | THR | A | 259 | −12.589 | 3.802 | −15.677 | 1.00 | 15.81 |
| ATOM | 2006 | C | THR | A | 259 | −16.433 | 4.248 | −15.173 | 1.00 | 14.21 |
| ATOM | 2007 | O | THR | A | 259 | −16.709 | 3.235 | −14.546 | 1.00 | 14.95 |
| ATOM | 2008 | N | PHE | A | 260 | −17.290 | 5.238 | −15.367 | 1.00 | 14.76 |
| ATOM | 2009 | CA | PHE | A | 260 | −18.691 | 5.194 | −14.926 | 1.00 | 15.13 |
| ATOM | 2010 | CB | PHE | A | 260 | −19.377 | 6.492 | −15.379 | 1.00 | 15.81 |
| ATOM | 2011 | CG | PHE | A | 260 | −20.886 | 6.508 | −15.228 | 1.00 | 15.47 |
| ATOM | 2012 | CD1 | PHE | A | 260 | −21.505 | 6.188 | −14.015 | 1.00 | 17.59 |
| ATOM | 2013 | CE1 | PHE | A | 260 | −22.903 | 6.259 | −13.898 | 1.00 | 19.11 |
| ATOM | 2014 | CZ | PHE | A | 260 | −23.682 | 6.653 | −14.991 | 1.00 | 17.18 |
| ATOM | 2015 | CE2 | PHE | A | 260 | −23.082 | 6.994 | −16.178 | 1.00 | 18.04 |
| ATOM | 2016 | CD2 | PHE | A | 260 | −21.679 | 6.917 | −16.296 | 1.00 | 17.22 |
| ATOM | 2017 | C | PHE | A | 260 | −19.436 | 3.977 | −15.475 | 1.00 | 15.55 |
| ATOM | 2018 | O | PHE | A | 260 | −19.426 | 3.725 | −16.684 | 1.00 | 15.81 |
| ATOM | 2019 | N | ASP | A | 261 | −20.093 | 3.235 | −14.586 | 1.00 | 15.51 |
| ATOM | 2020 | CA | ASP | A | 261 | −21.008 | 2.176 | −15.006 | 1.00 | 16.05 |
| ATOM | 2021 | CB | ASP | A | 261 | −20.303 | 0.813 | −15.015 | 1.00 | 16.46 |
| ATOM | 2022 | CG | ASP | A | 261 | −21.205 | −0.321 | −15.490 | 1.00 | 17.60 |
| ATOM | 2023 | OD1 | ASP | A | 261 | −22.440 | −0.122 | −15.579 | 1.00 | 18.97 |
| ATOM | 2024 | OD2 | ASP | A | 261 | −20.656 | −1.404 | −15.810 | 1.00 | 18.29 |
| ATOM | 2025 | C | ASP | A | 261 | −22.117 | 2.185 | −13.972 | 1.00 | 16.30 |
| ATOM | 2026 | O | ASP | A | 261 | −21.882 | 1.809 | −12.840 | 1.00 | 15.53 |
| ATOM | 2027 | N | PRO | A | 262 | −23.320 | 2.610 | −14.374 | 1.00 | 18.21 |
| ATOM | 2028 | CA | PRO | A | 262 | −24.438 | 2.716 | −13.412 | 1.00 | 20.39 |
| ATOM | 2029 | CB | PRO | A | 262 | −25.589 | 3.308 | −14.247 | 1.00 | 20.43 |
| ATOM | 2030 | CG | PRO | A | 262 | −25.235 | 3.044 | −15.669 | 1.00 | 20.06 |
| ATOM | 2031 | CD | PRO | A | 262 | −23.709 | 2.994 | −15.734 | 1.00 | 17.40 |
| ATOM | 2032 | C | PRO | A | 262 | −24.815 | 1.382 | −12.753 | 1.00 | 22.31 |
| ATOM | 2033 | O | PRO | A | 262 | −25.356 | 1.374 | −11.622 | 1.00 | 22.24 |
| ATOM | 2034 | N | ASN | A | 263 | −24.508 | 0.267 | −13.421 | 1.00 | 22.99 |
| ATOM | 2035 | CA | ASN | A | 263 | −24.750 | −1.048 | −12.838 | 1.00 | 25.42 |
| ATOM | 2036 | CB | ASN | A | 263 | −24.574 | −2.149 | −13.890 | 1.00 | 26.62 |
| ATOM | 2037 | CG | ASN | A | 263 | −25.680 | −2.128 | −14.948 | 1.00 | 30.74 |
| ATOM | 2038 | OD1 | ASN | A | 263 | −26.688 | −1.419 | −14.814 | 1.00 | 35.92 |
| ATOM | 2039 | ND2 | ASN | A | 263 | −25.490 | −2.906 | −16.007 | 1.00 | 35.87 |
| ATOM | 2040 | C | ASN | A | 263 | −23.894 | −1.316 | −11.598 | 1.00 | 25.45 |
| ATOM | 2041 | O | ASN | A | 263 | −24.210 | −2.190 | −10.795 | 1.00 | 26.56 |
| ATOM | 2042 | N | LEU | A | 264 | −22.835 | −0.529 | −11.413 | 1.00 | 24.54 |
| ATOM | 2043 | CA | LEU | A | 264 | −22.022 | −0.616 | −10.213 | 1.00 | 24.27 |
| ATOM | 2044 | CB | LEU | A | 264 | −20.549 | −0.287 | −10.520 | 1.00 | 24.43 |
| ATOM | 2045 | CG | LEU | A | 264 | −19.752 | −1.346 | −11.288 | 1.00 | 25.38 |
| ATOM | 2046 | CD1 | LEU | A | 264 | −18.375 | −0.809 | −11.659 | 1.00 | 26.05 |
| ATOM | 2047 | CD2 | LEU | A | 264 | −19.619 | −2.672 | −10.523 | 1.00 | 26.24 |
| ATOM | 2048 | C | LEU | A | 264 | −22.542 | 0.273 | −9.066 | 1.00 | 23.47 |
| ATOM | 2049 | O | LEU | A | 264 | −21.956 | 0.292 | −7.988 | 1.00 | 23.97 |
| ATOM | 2050 | N | GLY | A | 265 | −23.631 | 1.000 | −9.292 | 1.00 | 23.32 |
| ATOM | 2051 | CA | GLY | A | 265 | −24.218 | 1.840 | −8.237 | 1.00 | 22.74 |
| ATOM | 2052 | C | GLY | A | 265 | −23.204 | 2.843 | −7.729 | 1.00 | 21.84 |
| ATOM | 2053 | O | GLY | A | 265 | −22.416 | 3.373 | −8.510 | 1.00 | 22.83 |
| ATOM | 2054 | N | CYS | A | 266 | −23.175 | 3.086 | −6.424 | 1.00 | 21.37 |
| ATOM | 2055 | CA | CYS | A | 266 | −22.233 | 4.073 | −5.883 | 1.00 | 21.00 |
| ATOM | 2056 | CB | CYS | A | 266 | −22.947 | 5.049 | −4.936 | 1.00 | 20.86 |
| ATOM | 2057 | SG | CYS | A | 266 | −24.347 | 5.912 | −5.711 | 1.00 | 20.96 |
| ATOM | 2058 | C | CYS | A | 266 | −20.992 | 3.427 | −5.275 | 1.00 | 20.98 |
| ATOM | 2059 | O | CYS | A | 266 | −20.513 | 3.814 | −4.203 | 1.00 | 21.01 |
| ATOM | 2060 | N | ASP | A | 267 | −20.462 | 2.443 | −6.002 | 1.00 | 20.39 |
| ATOM | 2061 | CA | ASP | A | 267 | −19.303 | 1.686 | −5.577 | 1.00 | 20.67 |
| ATOM | 2062 | CB | ASP | A | 267 | −18.961 | 0.621 | −6.618 | 1.00 | 20.84 |
| ATOM | 2063 | CG | ASP | A | 267 | −17.666 | −0.101 | −6.288 | 1.00 | 24.28 |
| ATOM | 2064 | OD1 | ASP | A | 267 | −16.852 | −0.322 | −7.200 | 1.00 | 25.84 |
| ATOM | 2065 | OD2 | ASP | A | 267 | −17.455 | −0.407 | −5.098 | 1.00 | 27.46 |
| ATOM | 2066 | C | ASP | A | 267 | −18.072 | 2.567 | −5.391 | 1.00 | 19.78 |
| ATOM | 2067 | O | ASP | A | 267 | −17.593 | 3.161 | −6.353 | 1.00 | 18.61 |
| ATOM | 2068 | N | ALA | A | 268 | −17.544 | 2.621 | −4.174 | 1.00 | 18.35 |
| ATOM | 2069 | CA | ALA | A | 268 | −16.315 | 3.395 | −3.944 | 1.00 | 19.35 |
| ATOM | 2070 | CB | ALA | A | 268 | −16.207 | 3.868 | −2.472 | 1.00 | 19.46 |
| ATOM | 2071 | C | ALA | A | 268 | −15.017 | 2.701 | −4.415 | 1.00 | 19.46 |
| ATOM | 2072 | O | ALA | A | 268 | −14.009 | 3.371 | −4.665 | 1.00 | 19.42 |
| ATOM | 2073 | N | GLY | A | 269 | −15.029 | 1.370 | −4.534 | 1.00 | 19.01 |
| ATOM | 2074 | CA | GLY | A | 269 | −13.826 | 0.644 | −4.936 | 1.00 | 19.17 |
| ATOM | 2075 | C | GLY | A | 269 | −13.370 | 1.032 | −6.343 | 1.00 | 18.75 |
| ATOM | 2076 | O | GLY | A | 269 | −12.175 | 1.134 | −6.624 | 1.00 | 19.54 |
| ATOM | 2077 | N | THR | A | 270 | −14.330 | 1.257 | −7.230 | 1.00 | 17.92 |
| ATOM | 2078 | CA | THR | A | 270 | −14.016 | 1.662 | −8.594 | 1.00 | 18.35 |
| ATOM | 2079 | CB | THR | A | 270 | −14.852 | 0.882 | −9.616 | 1.00 | 18.43 |
| ATOM | 2080 | OG1 | THR | A | 270 | −16.246 | 1.085 | −9.350 | 1.00 | 18.51 |
| ATOM | 2081 | CG2 | THR | A | 270 | −14.529 | −0.626 | −9.555 | 1.00 | 19.75 |
| ATOM | 2082 | C | THR | A | 270 | −14.261 | 3.172 | −8.771 | 1.00 | 18.25 |
| ATOM | 2083 | O | THR | A | 270 | −14.326 | 3.674 | −9.904 | 1.00 | 17.88 |
| ATOM | 2084 | N | PHE | A | 271 | −14.434 | 3.880 | −7.650 | 1.00 | 17.17 |
| ATOM | 2085 | CA | PHE | A | 271 | −14.531 | 5.359 | −7.656 | 1.00 | 17.38 |
| ATOM | 2086 | CB | PHE | A | 271 | −13.183 | 5.965 | −8.121 | 1.00 | 17.67 |
| ATOM | 2087 | CG | PHE | A | 271 | −12.946 | 7.376 | −7.673 | 1.00 | 21.97 |
| ATOM | 2088 | CD1 | PHE | A | 271 | −12.656 | 7.653 | −6.337 | 1.00 | 24.90 |
| ATOM | 2089 | CE1 | PHE | A | 271 | −12.447 | 8.981 | −5.923 | 1.00 | 24.46 |
| ATOM | 2090 | CZ | PHE | A | 271 | −12.474 | 10.043 | −6.863 | 1.00 | 23.20 |
| ATOM | 2091 | CE2 | PHE | A | 271 | −12.733 | 9.783 | −8.196 | 1.00 | 21.92 |
| ATOM | 2092 | CD2 | PHE | A | 271 | −12.956 | 8.436 | −8.599 | 1.00 | 24.30 |
| ATOM | 2093 | C | PHE | A | 271 | −15.677 | 5.856 | −8.551 | 1.00 | 16.63 |
| ATOM | 2094 | O | PHE | A | 271 | −15.479 | 6.764 | −9.358 | 1.00 | 15.93 |
| ATOM | 2095 | N | GLN | A | 272 | −16.861 | 5.249 | −8.439 | 1.00 | 15.21 |
| ATOM | 2096 | CA | GLN | A | 272 | −18.011 | 5.673 | −9.251 | 1.00 | 14.97 |
| ATOM | 2097 | CB | GLN | A | 272 | −19.227 | 4.755 | −9.013 | 1.00 | 14.93 |
| ATOM | 2098 | CG | GLN | A | 272 | −19.021 | 3.355 | −9.615 | 1.00 | 16.30 |
| ATOM | 2099 | CD | GLN | A | 272 | −18.755 | 3.413 | −11.102 | 1.00 | 15.81 |
| ATOM | 2100 | OE1 | GLN | A | 272 | −19.575 | 3.909 | −11.883 | 1.00 | 16.97 |
| ATOM | 2101 | NE2 | GLN | A | 272 | −17.617 | 2.861 | −11.512 | 1.00 | 18.50 |
| ATOM | 2102 | C | GLN | A | 272 | −18.402 | 7.118 | −8.929 | 1.00 | 14.73 |
| ATOM | 2103 | O | GLN | A | 272 | −18.194 | 7.555 | −7.800 | 1.00 | 15.60 |
| ATOM | 2104 | N | PRO | A | 273 | −18.955 | 7.859 | −9.914 | 1.00 | 14.45 |
| ATOM | 2105 | CA | PRO | A | 273 | −19.342 | 9.255 | −9.682 | 1.00 | 14.57 |
| ATOM | 2106 | CB | PRO | A | 273 | −20.157 | 9.597 | −10.927 | 1.00 | 14.64 |
| ATOM | 2107 | CG | PRO | A | 273 | −19.443 | 8.767 | −12.031 | 1.00 | 14.70 |
| ATOM | 2108 | CD | PRO | A | 273 | −19.156 | 7.458 | −11.326 | 1.00 | 14.10 |
| ATOM | 2109 | C | PRO | A | 273 | −20.162 | 9.542 | −8.407 | 1.00 | 15.22 |
| ATOM | 2110 | O | PRO | A | 273 | −19.910 | 10.562 | −7.752 | 1.00 | 15.03 |
| ATOM | 2111 | N | CYS | A | 274 | −21.130 | 8.682 | −8.075 | 1.00 | 15.76 |
| ATOM | 2112 | CA | CYS | A | 274 | −21.926 | 8.913 | −6.853 | 1.00 | 16.22 |
| ATOM | 2113 | CB | CYS | A | 274 | −23.389 | 8.489 | −7.039 | 1.00 | 16.57 |
| ATOM | 2114 | SG | CYS | A | 274 | −23.611 | 6.769 | −7.423 | 1.00 | 17.39 |
| ATOM | 2115 | C | CYS | A | 274 | −21.331 | 8.281 | −5.605 | 1.00 | 16.64 |
| ATOM | 2116 | O | CYS | A | 274 | −21.958 | 8.329 | −4.529 | 1.00 | 16.55 |
| ATOM | 2117 | N | SER | A | 275 | −20.137 | 7.681 | −5.715 | 1.00 | 15.61 |
| ATOM | 2118 | CA | SER | A | 275 | −19.476 | 7.117 | −4.528 | 1.00 | 15.81 |
| ATOM | 2119 | CB | SER | A | 275 | −18.244 | 6.253 | −4.877 | 1.00 | 15.06 |
| ATOM | 2120 | OG | SER | A | 275 | −17.144 | 7.041 | −5.315 | 1.00 | 14.92 |
| ATOM | 2121 | C | SER | A | 275 | −19.097 | 8.232 | −3.545 | 1.00 | 16.06 |
| ATOM | 2122 | O | SER | A | 275 | −18.818 | 9.366 | −3.949 | 1.00 | 14.39 |
| ATOM | 2123 | N | ASP | A | 276 | −19.103 | 7.919 | −2.248 | 1.00 | 16.42 |
| ATOM | 2124 | CA | ASP | A | 276 | −18.731 | 8.935 | −1.271 | 1.00 | 16.52 |
| ATOM | 2125 | CB | ASP | A | 276 | −19.020 | 8.511 | 0.189 | 1.00 | 16.27 |
| ATOM | 2126 | CG | ASP | A | 276 | −18.244 | 7.281 | 0.656 | 1.00 | 19.14 |
| ATOM | 2127 | OD1 | ASP | A | 276 | −18.371 | 7.001 | 1.873 | 1.00 | 19.62 |
| ATOM | 2128 | OD2 | ASP | A | 276 | −17.544 | 6.593 | −0.120 | 1.00 | 17.10 |
| ATOM | 2129 | C | ASP | A | 276 | −17.312 | 9.469 | −1.492 | 1.00 | 16.17 |
| ATOM | 2130 | O | ASP | A | 276 | −17.084 | 10.683 | −1.415 | 1.00 | 15.20 |
| ATOM | 2131 | N | LYS | A | 277 | −16.381 | 8.577 | −1.823 | 1.00 | 15.43 |
| ATOM | 2132 | CA | LYS | A | 277 | −14.994 | 8.982 | −2.115 | 1.00 | 15.34 |
| ATOM | 2133 | CB | LYS | A | 277 | −14.089 | 7.763 | −2.326 | 1.00 | 15.23 |
| ATOM | 2134 | CG | LYS | A | 277 | −13.924 | 6.905 | −1.059 | 1.00 | 17.01 |
| ATOM | 2135 | CD | LYS | A | 277 | −12.752 | 5.929 | −1.204 | 1.00 | 21.20 |
| ATOM | 2136 | CE | LYS | A | 277 | −12.662 | 5.017 | 0.015 | 1.00 | 22.94 |
| ATOM | 2137 | NZ | LYS | A | 277 | −11.533 | 4.067 | −0.165 | 1.00 | 29.19 |
| ATOM | 2138 | C | LYS | A | 277 | −14.900 | 9.915 | −3.324 | 1.00 | 14.30 |
| ATOM | 2139 | O | LYS | A | 277 | −14.152 | 10.887 | −3.288 | 1.00 | 14.70 |
| ATOM | 2140 | N | ALA | A | 278 | −15.644 | 9.620 | −4.393 | 1.00 | 14.45 |
| ATOM | 2141 | CA | ALA | A | 278 | −15.588 | 10.464 | −5.605 | 1.00 | 13.61 |
| ATOM | 2142 | CB | ALA | A | 278 | −16.250 | 9.775 | −6.783 | 1.00 | 13.30 |
| ATOM | 2143 | C | ALA | A | 278 | −16.210 | 11.827 | −5.357 | 1.00 | 13.50 |
| ATOM | 2144 | O | ALA | A | 278 | −15.730 | 12.840 | −5.864 | 1.00 | 13.02 |
| ATOM | 2145 | N | LEU | A | 279 | −17.283 | 11.855 | −4.565 | 1.00 | 13.22 |
| ATOM | 2146 | CA | LEU | A | 279 | −17.936 | 13.132 | −4.239 | 1.00 | 12.92 |
| ATOM | 2147 | CB | LEU | A | 279 | −19.323 | 12.893 | −3.625 | 1.00 | 13.21 |
| ATOM | 2148 | CG | LEU | A | 279 | −20.384 | 12.358 | −4.601 | 1.00 | 13.94 |
| ATOM | 2149 | CD1 | LEU | A | 279 | −21.707 | 11.969 | −3.887 | 1.00 | 17.68 |
| ATOM | 2150 | CD2 | LEU | A | 279 | −20.653 | 13.319 | −5.781 | 1.00 | 17.52 |
| ATOM | 2151 | C | LEU | A | 279 | −17.065 | 13.995 | −3.348 | 1.00 | 12.84 |
| ATOM | 2152 | O | LEU | A | 279 | −16.941 | 15.203 | −3.577 | 1.00 | 13.54 |
| ATOM | 2153 | N | SER | A | 280 | −16.463 | 13.390 | −2.315 | 1.00 | 12.45 |
| ATOM | 2154 | CA | SER | A | 280 | −15.502 | 14.106 | −1.459 | 1.00 | 13.53 |
| ATOM | 2155 | CB | SER | A | 280 | −14.951 | 13.168 | −0.364 | 1.00 | 13.65 |
| ATOM | 2156 | OG | SER | A | 280 | −14.008 | 13.863 | 0.468 | 1.00 | 15.07 |
| ATOM | 2157 | C | SER | A | 280 | −14.332 | 14.672 | −2.285 | 1.00 | 14.09 |
| ATOM | 2158 | O | SER | A | 280 | −13.925 | 15.856 | −2.130 | 1.00 | 13.43 |
| ATOM | 2159 | N | ASN | A | 281 | −13.795 | 13.830 | −3.166 | 1.00 | 13.27 |
| ATOM | 2160 | CA | ASN | A | 281 | −12.690 | 14.257 | −4.027 | 1.00 | 13.19 |
| ATOM | 2161 | CB | ASN | A | 281 | −12.239 | 13.078 | −4.888 | 1.00 | 12.05 |
| ATOM | 2162 | CG | ASN | A | 281 | −11.116 | 13.455 | −5.849 | 1.00 | 13.13 |
| ATOM | 2163 | OD1 | ASN | A | 281 | −9.989 | 13.637 | −5.446 | 1.00 | 13.26 |
| ATOM | 2164 | ND2 | ASN | A | 281 | −11.442 | 13.573 | −7.124 | 1.00 | 11.63 |
| ATOM | 2165 | C | ASN | A | 281 | −13.096 | 15.432 | −4.933 | 1.00 | 12.33 |
| ATOM | 2166 | O | ASN | A | 281 | −12.330 | 16.380 | −5.109 | 1.00 | 13.49 |
| ATOM | 2167 | N | LEU | A | 282 | −14.287 | 15.355 | −5.506 | 1.00 | 12.05 |
| ATOM | 2168 | CA | LEU | A | 282 | −14.760 | 16.422 | −6.376 | 1.00 | 13.21 |
| ATOM | 2169 | CB | LEU | A | 282 | −16.147 | 16.109 | −6.949 | 1.00 | 12.17 |
| ATOM | 2170 | CG | LEU | A | 282 | −16.791 | 17.216 | −7.820 | 1.00 | 14.57 |
| ATOM | 2171 | CD1 | LEU | A | 282 | −16.011 | 17.378 | −9.126 | 1.00 | 16.58 |
| ATOM | 2172 | CD2 | LEU | A | 282 | −18.241 | 16.863 | −8.170 | 1.00 | 15.68 |
| ATOM | 2173 | C | LEU | A | 282 | −14.739 | 17.754 | −5.638 | 1.00 | 12.69 |
| ATOM | 2174 | O | LEU | A | 282 | −14.201 | 18.735 | −6.153 | 1.00 | 13.45 |
| ATOM | 2175 | N | LYS | A | 283 | −15.283 | 17.791 | −4.415 | 1.00 | 12.75 |
| ATOM | 2176 | CA | LYS | A | 283 | −15.306 | 19.026 | −3.656 | 1.00 | 12.89 |
| ATOM | 2177 | CB | LYS | A | 283 | −16.079 | 18.860 | −2.334 | 1.00 | 12.90 |
| ATOM | 2178 | CG | LYS | A | 283 | −15.912 | 20.089 | −1.432 | 1.00 | 13.94 |
| ATOM | 2179 | CD | LYS | A | 283 | −16.909 | 20.076 | −0.252 | 1.00 | 14.67 |
| ATOM | 2180 | CE | LYS | A | 283 | −16.530 | 21.136 | 0.797 | 1.00 | 13.67 |
| ATOM | 2181 | NZ | LYS | A | 283 | −16.315 | 22.489 | 0.212 | 1.00 | 19.03 |
| ATOM | 2182 | C | LYS | A | 283 | −13.889 | 19.537 | −3.385 | 1.00 | 12.43 |
| ATOM | 2183 | O | LYS | A | 283 | −13.612 | 20.710 | −3.556 | 1.00 | 12.14 |
| ATOM | 2184 | N | VAL | A | 284 | −12.988 | 18.652 | −2.966 | 1.00 | 12.02 |
| ATOM | 2185 | CA | VAL | A | 284 | −11.624 | 19.055 | −2.633 | 1.00 | 12.77 |
| ATOM | 2186 | CB | VAL | A | 284 | −10.845 | 17.875 | −2.014 | 1.00 | 13.17 |
| ATOM | 2187 | CG1 | VAL | A | 284 | −9.320 | 18.169 | −1.936 | 1.00 | 13.21 |
| ATOM | 2188 | CG2 | VAL | A | 284 | −11.391 | 17.557 | −0.630 | 1.00 | 15.81 |
| ATOM | 2189 | C | VAL | A | 284 | −10.927 | 19.599 | −3.881 | 1.00 | 12.74 |
| ATOM | 2190 | O | VAL | A | 284 | −10.228 | 20.636 | −3.827 | 1.00 | 12.21 |
| ATOM | 2191 | N | VAL | A | 285 | −11.153 | 18.927 | −5.012 | 1.00 | 11.54 |
| ATOM | 2192 | CA | VAL | A | 285 | −10.560 | 19.389 | −6.287 | 1.00 | 12.35 |
| ATOM | 2193 | CB | VAL | A | 285 | −10.694 | 18.330 | −7.425 | 1.00 | 12.36 |
| ATOM | 2194 | CG1 | VAL | A | 285 | −10.316 | 18.944 | −8.813 | 1.00 | 12.25 |
| ATOM | 2195 | CG2 | VAL | A | 285 | −9.795 | 17.104 | −7.140 | 1.00 | 13.25 |
| ATOM | 2196 | C | VAL | A | 285 | −11.130 | 20.770 | −6.712 | 1.00 | 12.08 |
| ATOM | 2197 | O | VAL | A | 285 | −10.367 | 21.696 | −6.989 | 1.00 | 12.60 |
| ATOM | 2198 | N | VAL | A | 286 | −12.452 | 20.913 | −6.728 | 1.00 | 11.87 |
| ATOM | 2199 | CA | VAL | A | 286 | −13.089 | 22.196 | −7.074 | 1.00 | 12.87 |
| ATOM | 2200 | CB | VAL | A | 286 | −14.631 | 22.080 | −7.038 | 1.00 | 13.01 |
| ATOM | 2201 | CG1 | VAL | A | 286 | −15.300 | 23.468 | −7.140 | 1.00 | 14.31 |
| ATOM | 2202 | CG2 | VAL | A | 286 | −15.103 | 21.157 | −8.200 | 1.00 | 14.42 |
| ATOM | 2203 | C | VAL | A | 286 | −12.586 | 23.324 | −6.164 | 1.00 | 12.84 |
| ATOM | 2204 | O | VAL | A | 286 | −12.206 | 24.402 | −6.635 | 1.00 | 13.75 |
| ATOM | 2205 | N | ASP | A | 287 | −12.552 | 23.064 | −4.853 | 1.00 | 12.85 |
| ATOM | 2206 | CA | ASP | A | 287 | −12.116 | 24.059 | −3.870 | 1.00 | 13.90 |
| ATOM | 2207 | CB | ASP | A | 287 | −12.199 | 23.506 | −2.440 | 1.00 | 13.12 |
| ATOM | 2208 | CG | ASP | A | 287 | −13.637 | 23.441 | −1.924 | 1.00 | 16.00 |
| ATOM | 2209 | OD1 | ASP | A | 287 | −14.541 | 24.002 | −2.583 | 1.00 | 16.20 |
| ATOM | 2210 | OD2 | ASP | A | 287 | −13.857 | 22.835 | −0.858 | 1.00 | 16.76 |
| ATOM | 2211 | C | ASP | A | 287 | −10.727 | 24.564 | −4.136 | 1.00 | 14.28 |
| ATOM | 2212 | O | ASP | A | 287 | −10.425 | 25.722 | −3.841 | 1.00 | 15.53 |
| ATOM | 2213 | N | SER | A | 288 | −9.862 | 23.709 | −4.677 | 1.00 | 14.50 |
| ATOM | 2214 | CA | SER | A | 288 | −8.478 | 24.093 | −4.949 | 1.00 | 14.58 |
| ATOM | 2215 | CB | SER | A | 288 | −7.625 | 22.843 | −5.229 | 1.00 | 14.15 |
| ATOM | 2216 | OG | SER | A | 288 | −7.758 | 22.417 | −6.565 | 1.00 | 13.73 |
| ATOM | 2217 | C | SER | A | 288 | −8.326 | 25.186 | −6.038 | 1.00 | 14.61 |
| ATOM | 2218 | O | SER | A | 288 | −7.274 | 25.847 | −6.143 | 1.00 | 14.59 |
| ATOM | 2219 | N | PHE | A | 289 | −9.392 | 25.416 | −6.809 | 1.00 | 13.99 |
| ATOM | 2220 | CA | PHE | A | 289 | −9.419 | 26.447 | −7.831 | 1.00 | 14.09 |
| ATOM | 2221 | CB | PHE | A | 289 | −9.994 | 25.882 | −9.135 | 1.00 | 13.52 |
| ATOM | 2222 | CG | PHE | A | 289 | −9.169 | 24.807 | −9.704 | 1.00 | 11.38 |
| ATOM | 2223 | CD1 | PHE | A | 289 | −7.976 | 25.114 | −10.367 | 1.00 | 12.17 |
| ATOM | 2224 | CE1 | PHE | A | 289 | −7.184 | 24.095 | −10.905 | 1.00 | 13.93 |
| ATOM | 2225 | CZ | PHE | A | 289 | −7.572 | 22.783 | −10.771 | 1.00 | 13.96 |
| ATOM | 2226 | CE2 | PHE | A | 289 | −8.756 | 22.452 | −10.097 | 1.00 | 11.77 |
| ATOM | 2227 | CD2 | PHE | A | 289 | −9.555 | 23.472 | −9.571 | 1.00 | 11.08 |
| ATOM | 2228 | C | PHE | A | 289 | −10.219 | 27.698 | −7.491 | 1.00 | 14.73 |
| ATOM | 2229 | O | PHE | A | 289 | −10.092 | 28.713 | −8.189 | 1.00 | 14.71 |
| ATOM | 2230 | N | ARG | A | 290 | −11.054 | 27.621 | −6.464 | 1.00 | 15.09 |
| ATOM | 2231 | CA | ARG | A | 290 | −11.953 | 28.740 | −6.140 | 1.00 | 16.64 |
| ATOM | 2232 | CB | ARG | A | 290 | −12.842 | 28.401 | −4.936 | 1.00 | 15.99 |
| ATOM | 2233 | CG | ARG | A | 290 | −13.913 | 27.375 | −5.230 | 1.00 | 15.65 |
| ATOM | 2234 | CD | ARG | A | 290 | −14.821 | 27.163 | −4.012 | 1.00 | 16.79 |
| ATOM | 2235 | NE | ARG | A | 290 | −15.843 | 26.172 | −4.330 | 1.00 | 15.04 |
| ATOM | 2236 | CZ | ARG | A | 290 | −16.986 | 26.470 | −4.933 | 1.00 | 17.22 |
| ATOM | 2237 | NH1 | ARG | A | 290 | −17.248 | 27.734 | −5.243 | 1.00 | 15.41 |
| ATOM | 2238 | NH2 | ARG | A | 290 | −17.855 | 25.511 | −5.239 | 1.00 | 15.94 |
| ATOM | 2239 | C | ARG | A | 290 | −11.240 | 30.046 | −5.864 | 1.00 | 18.04 |
| ATOM | 2240 | O | ARG | A | 290 | −11.690 | 31.125 | −6.279 | 1.00 | 19.61 |
| ATOM | 2241 | N | SER | A | 291 | −10.150 | 29.984 | −5.128 | 1.00 | 19.44 |
| ATOM | 2242 | CA | SER | A | 291 | −9.571 | 31.246 | −4.667 | 1.00 | 21.57 |
| ATOM | 2243 | CB | SER | A | 291 | −9.146 | 31.101 | −3.212 | 1.00 | 22.18 |
| ATOM | 2244 | OG | SER | A | 291 | −7.998 | 30.284 | −3.144 | 1.00 | 28.35 |
| ATOM | 2245 | C | SER | A | 291 | −8.423 | 31.762 | −5.534 | 1.00 | 20.65 |
| ATOM | 2246 | O | SER | A | 291 | −7.865 | 32.851 | −5.272 | 1.00 | 22.43 |
| ATOM | 2247 | N | ILE | A | 292 | −8.066 | 31.019 | −6.576 | 1.00 | 19.16 |
| ATOM | 2248 | CA | ILE | A | 292 | −6.855 | 31.367 | −7.330 | 1.00 | 17.65 |
| ATOM | 2249 | CB | ILE | A | 292 | −5.805 | 30.185 | −7.408 | 1.00 | 18.07 |
| ATOM | 2250 | CG1 | ILE | A | 292 | −6.379 | 28.972 | −8.194 | 1.00 | 17.67 |
| ATOM | 2251 | CD1 | ILE | A | 292 | −5.315 | 27.924 | −8.649 | 1.00 | 17.27 |
| ATOM | 2252 | CG2 | ILE | A | 292 | −5.341 | 29.795 | −5.994 | 1.00 | 18.29 |
| ATOM | 2253 | C | ILE | A | 292 | −7.065 | 31.973 | −8.708 | 1.00 | 17.20 |
| ATOM | 2254 | O | ILE | A | 292 | −6.136 | 32.563 | −9.251 | 1.00 | 16.35 |
| ATOM | 2255 | N | TYR | A | 293 | −8.252 | 31.797 | −9.290 | 1.00 | 15.85 |
| ATOM | 2256 | CA | TYR | A | 293 | −8.509 | 32.304 | −10.648 | 1.00 | 15.83 |
| ATOM | 2257 | CB | TYR | A | 293 | −9.301 | 31.270 | −11.474 | 1.00 | 15.43 |
| ATOM | 2258 | CG | TYR | A | 293 | −8.571 | 30.014 | −11.886 | 1.00 | 15.10 |
| ATOM | 2259 | CD1 | TYR | A | 293 | −7.183 | 29.960 | −11.965 | 1.00 | 14.38 |
| ATOM | 2260 | CE1 | TYR | A | 293 | −6.540 | 28.795 | −12.395 | 1.00 | 14.38 |
| ATOM | 2261 | CZ | TYR | A | 293 | −7.306 | 27.685 | −12.743 | 1.00 | 14.90 |
| ATOM | 2262 | OH | TYR | A | 293 | −6.700 | 26.522 | −13.158 | 1.00 | 15.55 |
| ATOM | 2263 | CE2 | TYR | A | 293 | −8.670 | 27.722 | −12.671 | 1.00 | 15.47 |
| ATOM | 2264 | CD2 | TYR | A | 293 | −9.298 | 28.875 | −12.255 | 1.00 | 13.91 |
| ATOM | 2265 | C | TYR | A | 293 | −9.351 | 33.581 | −10.591 | 1.00 | 15.69 |
| ATOM | 2266 | O | TYR | A | 293 | −10.404 | 33.594 | −9.942 | 1.00 | 15.47 |
| ATOM | 2267 | N | GLY | A | 294 | −8.892 | 34.629 | −11.276 | 1.00 | 14.83 |
| ATOM | 2268 | CA | GLY | A | 294 | −9.641 | 35.899 | −11.353 | 1.00 | 15.57 |
| ATOM | 2269 | C | GLY | A | 294 | −11.078 | 35.702 | −11.858 | 1.00 | 15.93 |
| ATOM | 2270 | O | GLY | A | 294 | −12.010 | 36.359 | −11.376 | 1.00 | 15.66 |
| ATOM | 2271 | N | VAL | A | 295 | −11.288 | 34.773 | −12.799 | 1.00 | 15.60 |
| ATOM | 2272 | CA | VAL | A | 295 | −12.651 | 34.520 | −13.270 | 1.00 | 16.24 |
| ATOM | 2273 | CB | VAL | A | 295 | −12.753 | 33.561 | −14.501 | 1.00 | 16.31 |
| ATOM | 2274 | CG1 | VAL | A | 295 | −12.170 | 34.195 | −15.740 | 1.00 | 16.26 |
| ATOM | 2275 | CG2 | VAL | A | 295 | −12.128 | 32.184 | −14.199 | 1.00 | 16.19 |
| ATOM | 2276 | C | VAL | A | 295 | −13.596 | 34.013 | −12.172 | 1.00 | 16.97 |
| ATOM | 2277 | O | VAL | A | 295 | −14.813 | 34.108 | −12.320 | 1.00 | 18.03 |
| ATOM | 2278 | N | ASN | A | 296 | −13.047 | 33.463 | −11.092 | 1.00 | 16.93 |
| ATOM | 2279 | CA | ASN | A | 296 | −13.878 | 32.920 | −10.020 | 1.00 | 17.69 |
| ATOM | 2280 | CB | ASN | A | 296 | −13.250 | 31.633 | −9.472 | 1.00 | 17.57 |
| ATOM | 2281 | CG | ASN | A | 296 | −13.296 | 30.493 | −10.482 | 1.00 | 16.44 |
| ATOM | 2282 | OD1 | ASN | A | 296 | −14.158 | 30.481 | −11.356 | 1.00 | 17.29 |
| ATOM | 2283 | ND2 | ASN | A | 296 | −12.401 | 29.513 | −10.336 | 1.00 | 15.99 |
| ATOM | 2284 | C | ASN | A | 296 | −14.187 | 33.915 | −8.896 | 1.00 | 19.30 |
| ATOM | 2285 | O | ASN | A | 296 | −14.945 | 33.601 | −7.979 | 1.00 | 19.07 |
| ATOM | 2286 | N | LYS | A | 297 | −13.617 | 35.116 | −9.007 | 1.00 | 20.37 |
| ATOM | 2287 | CA | LYS | A | 297 | −13.811 | 36.203 | −8.038 | 1.00 | 22.43 |
| ATOM | 2288 | CB | LYS | A | 297 | −13.209 | 37.502 | −8.584 | 1.00 | 22.90 |
| ATOM | 2289 | CG | LYS | A | 297 | −11.741 | 37.680 | −8.316 | 1.00 | 30.03 |
| ATOM | 2290 | CD | LYS | A | 297 | −11.401 | 39.189 | −8.309 | 1.00 | 35.34 |
| ATOM | 2291 | CE | LYS | A | 297 | −12.247 | 39.913 | −7.255 | 1.00 | 39.86 |
| ATOM | 2292 | NZ | LYS | A | 297 | −11.995 | 41.386 | −7.178 | 1.00 | 42.72 |
| ATOM | 2293 | C | LYS | A | 297 | −15.275 | 36.453 | −7.782 | 1.00 | 21.89 |
| ATOM | 2294 | O | LYS | A | 297 | −16.061 | 36.585 | −8.712 | 1.00 | 21.96 |
| ATOM | 2295 | N | GLY | A | 298 | −15.659 | 36.537 | −6.517 | 1.00 | 22.72 |
| ATOM | 2296 | CA | GLY | A | 298 | −17.050 | 36.869 | −6.219 | 1.00 | 22.99 |
| ATOM | 2297 | C | GLY | A | 298 | −18.043 | 35.720 | −6.278 | 1.00 | 23.54 |
| ATOM | 2298 | O | GLY | A | 298 | −19.180 | 35.885 | −5.855 | 1.00 | 25.04 |
| ATOM | 2299 | N | ILE | A | 299 | −17.647 | 34.546 | −6.784 | 1.00 | 22.16 |
| ATOM | 2300 | CA | ILE | A | 299 | −18.574 | 33.393 | −6.763 | 1.00 | 21.47 |
| ATOM | 2301 | CB | ILE | A | 299 | −18.251 | 32.350 | −7.884 | 1.00 | 21.22 |
| ATOM | 2302 | CG1 | ILE | A | 299 | −18.356 | 32.985 | −9.274 | 1.00 | 19.64 |
| ATOM | 2303 | CD1 | ILE | A | 299 | −17.740 | 32.095 | −10.415 | 1.00 | 19.76 |
| ATOM | 2304 | CG2 | ILE | A | 299 | −19.163 | 31.091 | −7.762 | 1.00 | 20.54 |
| ATOM | 2305 | C | ILE | A | 299 | −18.562 | 32.740 | −5.375 | 1.00 | 22.34 |
| ATOM | 2306 | O | ILE | A | 299 | −17.486 | 32.395 | −4.861 | 1.00 | 22.29 |
| ATOM | 2307 | N | PRO | A | 300 | −19.743 | 32.580 | −4.751 | 1.00 | 23.04 |
| ATOM | 2308 | CA | PRO | A | 300 | −19.791 | 32.018 | −3.392 | 1.00 | 23.60 |
| ATOM | 2309 | CB | PRO | A | 300 | −21.217 | 32.364 | −2.922 | 1.00 | 24.19 |
| ATOM | 2310 | CG | PRO | A | 300 | −22.015 | 32.437 | −4.178 | 1.00 | 23.80 |
| ATOM | 2311 | CD | PRO | A | 300 | −21.085 | 32.934 | −5.253 | 1.00 | 23.06 |
| ATOM | 2312 | C | PRO | A | 300 | −19.584 | 30.500 | −3.322 | 1.00 | 23.50 |
| ATOM | 2313 | O | PRO | A | 300 | −19.664 | 29.810 | −4.347 | 1.00 | 22.46 |
| ATOM | 2314 | N | ALA | A | 301 | −19.325 | 29.985 | −2.116 | 1.00 | 22.68 |
| ATOM | 2315 | CA | ALA | A | 301 | −19.380 | 28.549 | −1.905 | 1.00 | 22.89 |
| ATOM | 2316 | CB | ALA | A | 301 | −18.988 | 28.185 | −0.465 | 1.00 | 23.54 |
| ATOM | 2317 | C | ALA | A | 301 | −20.788 | 28.074 | −2.236 | 1.00 | 21.91 |
| ATOM | 2318 | O | ALA | A | 301 | −21.759 | 28.834 | −2.108 | 1.00 | 23.09 |
| ATOM | 2319 | N | GLY | A | 302 | −20.898 | 26.838 | −2.698 | 1.00 | 20.96 |
| ATOM | 2320 | CA | GLY | A | 302 | −22.173 | 26.272 | −3.115 | 1.00 | 19.69 |
| ATOM | 2321 | C | GLY | A | 302 | −22.565 | 26.637 | −4.537 | 1.00 | 19.78 |
| ATOM | 2322 | O | GLY | A | 302 | −23.661 | 26.283 | −4.991 | 1.00 | 19.32 |
| ATOM | 2323 | N | ALA | A | 303 | −21.686 | 27.355 | −5.235 | 1.00 | 17.97 |
| ATOM | 2324 | CA | ALA | A | 303 | −21.948 | 27.708 | −6.635 | 1.00 | 17.13 |
| ATOM | 2325 | CB | ALA | A | 303 | −22.168 | 29.212 | −6.812 | 1.00 | 16.73 |
| ATOM | 2326 | C | ALA | A | 303 | −20.784 | 27.245 | −7.481 | 1.00 | 16.19 |
| ATOM | 2327 | O | ALA | A | 303 | −19.647 | 27.171 | −7.004 | 1.00 | 16.23 |
| ATOM | 2328 | N | ALA | A | 304 | −21.067 | 26.956 | −8.746 | 1.00 | 15.66 |
| ATOM | 2329 | CA | ALA | A | 304 | −20.069 | 26.378 | −9.640 | 1.00 | 15.10 |
| ATOM | 2330 | CB | ALA | A | 304 | −20.750 | 25.795 | −10.860 | 1.00 | 15.80 |
| ATOM | 2331 | C | ALA | A | 304 | −19.002 | 27.394 | −10.044 | 1.00 | 14.74 |
| ATOM | 2332 | O | ALA | A | 304 | −19.270 | 28.587 | −10.121 | 1.00 | 14.27 |
| ATOM | 2333 | N | VAL | A | 305 | −17.783 | 26.914 | −10.300 | 1.00 | 14.18 |
| ATOM | 2334 | CA | VAL | A | 305 | −16.680 | 27.783 | −10.698 | 1.00 | 13.54 |
| ATOM | 2335 | CB | VAL | A | 305 | −15.656 | 27.971 | −9.543 | 1.00 | 13.42 |
| ATOM | 2336 | CG1 | VAL | A | 305 | −16.224 | 28.881 | −8.418 | 1.00 | 14.25 |
| ATOM | 2337 | CG2 | VAL | A | 305 | −15.218 | 26.597 | −8.966 | 1.00 | 14.99 |
| ATOM | 2338 | C | VAL | A | 305 | −15.952 | 27.141 | −11.873 | 1.00 | 13.34 |
| ATOM | 2339 | O | VAL | A | 305 | −16.121 | 25.944 | −12.126 | 1.00 | 12.46 |
| ATOM | 2340 | N | ALA | A | 306 | −15.130 | 27.921 | −12.562 | 1.00 | 13.38 |
| ATOM | 2341 | CA | ALA | A | 306 | −14.233 | 27.376 | −13.573 | 1.00 | 14.37 |
| ATOM | 2342 | CB | ALA | A | 306 | −13.709 | 28.504 | −14.470 | 1.00 | 15.32 |
| ATOM | 2343 | C | ALA | A | 306 | −13.082 | 26.626 | −12.938 | 1.00 | 14.68 |
| ATOM | 2344 | O | ALA | A | 306 | −12.457 | 27.116 | −11.974 | 1.00 | 15.21 |
| ATOM | 2345 | N | ILE | A | 307 | −12.781 | 25.452 | −13.484 | 1.00 | 13.50 |
| ATOM | 2346 | CA | ILE | A | 307 | −11.667 | 24.668 | −12.975 | 1.00 | 13.67 |
| ATOM | 2347 | CB | ILE | A | 307 | −12.134 | 23.438 | −12.163 | 1.00 | 14.45 |
| ATOM | 2348 | CG1 | ILE | A | 307 | −12.756 | 22.386 | −13.072 | 1.00 | 15.37 |
| ATOM | 2349 | CD1 | ILE | A | 307 | −12.921 | 21.033 | −12.368 | 1.00 | 19.15 |
| ATOM | 2350 | CG2 | ILE | A | 307 | −13.119 | 23.848 | −11.005 | 1.00 | 15.52 |
| ATOM | 2351 | C | ILE | A | 307 | −10.646 | 24.290 | −14.059 | 1.00 | 12.52 |
| ATOM | 2352 | O | ILE | A | 307 | −10.974 | 24.232 | −15.264 | 1.00 | 12.22 |
| ATOM | 2353 | N | GLY | A | 308 | −9.405 | 24.095 | −13.604 | 1.00 | 11.71 |
| ATOM | 2354 | CA | GLY | A | 308 | −8.276 | 23.737 | −14.452 | 1.00 | 11.64 |
| ATOM | 2355 | C | GLY | A | 308 | −7.853 | 22.306 | −14.199 | 1.00 | 11.62 |
| ATOM | 2356 | O | GLY | A | 308 | −8.667 | 21.444 | −13.806 | 1.00 | 12.05 |
| ATOM | 2357 | N | ARG | A | 309 | −6.583 | 22.026 | −14.454 | 1.00 | 11.39 |
| ATOM | 2358 | CA | ARG | A | 309 | −6.091 | 20.661 | −14.337 | 1.00 | 11.23 |
| ATOM | 2359 | CB | ARG | A | 309 | −4.896 | 20.467 | −15.275 | 1.00 | 11.37 |
| ATOM | 2360 | CG | ARG | A | 309 | −5.220 | 20.697 | −16.791 | 1.00 | 11.29 |
| ATOM | 2361 | CD | ARG | A | 309 | −4.066 | 20.130 | −17.625 | 1.00 | 12.62 |
| ATOM | 2362 | NE | ARG | A | 309 | −2.845 | 20.919 | −17.425 | 1.00 | 12.15 |
| ATOM | 2363 | CZ | ARG | A | 309 | −1.701 | 20.665 | −18.047 | 1.00 | 15.00 |
| ATOM | 2364 | NH1 | ARG | A | 309 | −1.630 | 19.633 | −18.910 | 1.00 | 12.05 |
| ATOM | 2365 | NH2 | ARG | A | 309 | −0.624 | 21.395 | −17.778 | 1.00 | 13.85 |
| ATOM | 2366 | C | ARG | A | 309 | −5.654 | 20.425 | −12.888 | 1.00 | 11.83 |
| ATOM | 2367 | O | ARG | A | 309 | −6.093 | 19.481 | −12.221 | 1.00 | 11.38 |
| ATOM | 2368 | N | TYR | A | 310 | −4.806 | 21.322 | −12.399 | 1.00 | 11.88 |
| ATOM | 2369 | CA | TYR | A | 310 | −4.293 | 21.215 | −11.022 | 1.00 | 11.17 |
| ATOM | 2370 | CB | TYR | A | 310 | −3.225 | 20.082 | −10.878 | 1.00 | 12.49 |
| ATOM | 2371 | CG | TYR | A | 310 | −2.065 | 20.201 | −11.844 | 1.00 | 13.10 |
| ATOM | 2372 | CD1 | TYR | A | 310 | −2.128 | 19.622 | −13.138 | 1.00 | 12.57 |
| ATOM | 2373 | CE1 | TYR | A | 310 | −1.069 | 19.772 | −14.039 | 1.00 | 15.63 |
| ATOM | 2374 | CZ | TYR | A | 310 | 0.065 | 20.475 | −13.649 | 1.00 | 14.31 |
| ATOM | 2375 | OH | TYR | A | 310 | 1.119 | 20.611 | −14.529 | 1.00 | 14.42 |
| ATOM | 2376 | CE2 | TYR | A | 310 | 0.159 | 21.030 | −12.379 | 1.00 | 12.68 |
| ATOM | 2377 | CD2 | TYR | A | 310 | −0.909 | 20.906 | −11.485 | 1.00 | 14.04 |
| ATOM | 2378 | C | TYR | A | 310 | −3.779 | 22.596 | −10.644 | 1.00 | 12.74 |
| ATOM | 2379 | O | TYR | A | 310 | −3.333 | 23.356 | −11.505 | 1.00 | 12.44 |
| ATOM | 2380 | N | ALA | A | 311 | −3.872 | 22.945 | −9.362 | 1.00 | 11.99 |
| ATOM | 2381 | CA | ALA | A | 311 | −3.618 | 24.337 | −8.975 | 1.00 | 13.33 |
| ATOM | 2382 | CB | ALA | A | 311 | −4.084 | 24.589 | −7.508 | 1.00 | 12.88 |
| ATOM | 2383 | C | ALA | A | 311 | −2.157 | 24.768 | −9.197 | 1.00 | 13.50 |
| ATOM | 2384 | O | ALA | A | 311 | −1.906 | 25.951 | −9.468 | 1.00 | 14.52 |
| ATOM | 2385 | N | GLU | A | 312 | −1.216 | 23.823 | −9.140 | 1.00 | 13.52 |
| ATOM | 2386 | CA | GLU | A | 312 | 0.219 | 24.134 | −9.332 | 1.00 | 14.23 |
| ATOM | 2387 | CB | GLU | A | 312 | 1.111 | 23.020 | −8.790 | 1.00 | 15.44 |
| ATOM | 2388 | CG | GLU | A | 312 | 0.933 | 22.802 | −7.303 | 1.00 | 16.54 |
| ATOM | 2389 | CD | GLU | A | 312 | −0.130 | 21.762 | −6.950 | 1.00 | 18.91 |
| ATOM | 2390 | OE1 | GLU | A | 312 | −0.941 | 21.338 | −7.808 | 1.00 | 16.89 |
| ATOM | 2391 | OE2 | GLU | A | 312 | −0.150 | 21.345 | −5.778 | 1.00 | 18.72 |
| ATOM | 2392 | C | GLU | A | 312 | 0.591 | 24.380 | −10.796 | 1.00 | 14.80 |
| ATOM | 2393 | O | GLU | A | 312 | 1.741 | 24.736 | −11.100 | 1.00 | 14.98 |
| ATOM | 2394 | N | ASP | A | 313 | −0.374 | 24.197 | −11.697 | 1.00 | 13.37 |
| ATOM | 2395 | CA | ASP | A | 313 | −0.112 | 24.258 | −13.155 | 1.00 | 13.71 |
| ATOM | 2396 | CB | ASP | A | 313 | −1.457 | 24.079 | −13.888 | 1.00 | 12.88 |
| ATOM | 2397 | CG | ASP | A | 313 | −1.320 | 23.671 | −15.343 | 1.00 | 14.44 |
| ATOM | 2398 | OD1 | ASP | A | 313 | −0.197 | 23.597 | −15.900 | 1.00 | 13.19 |
| ATOM | 2399 | OD2 | ASP | A | 313 | −2.400 | 23.406 | −15.923 | 1.00 | 13.61 |
| ATOM | 2400 | C | ASP | A | 313 | 0.512 | 25.589 | −13.587 | 1.00 | 14.00 |
| ATOM | 2401 | O | ASP | A | 313 | 0.007 | 26.662 | −13.219 | 1.00 | 14.35 |
| ATOM | 2402 | N | VAL | A | 314 | 1.577 | 25.530 | −14.399 | 1.00 | 13.84 |
| ATOM | 2403 | CA | VAL | A | 314 | 2.145 | 26.747 | −14.988 | 1.00 | 15.19 |
| ATOM | 2404 | CB | VAL | A | 314 | 3.602 | 27.016 | −14.520 | 1.00 | 16.70 |
| ATOM | 2405 | CG1 | VAL | A | 314 | 3.638 | 27.295 | −13.009 | 1.00 | 17.94 |
| ATOM | 2406 | CG2 | VAL | A | 314 | 4.551 | 25.857 | −14.915 | 1.00 | 16.69 |
| ATOM | 2407 | C | VAL | A | 314 | 2.123 | 26.729 | −16.528 | 1.00 | 15.30 |
| ATOM | 2408 | O | VAL | A | 314 | 2.712 | 27.598 | −17.165 | 1.00 | 15.13 |
| ATOM | 2409 | N | TYR | A | 315 | 1.441 | 25.743 | −17.111 | 1.00 | 14.51 |
| ATOM | 2410 | CA | TYR | A | 315 | 1.351 | 25.634 | −18.580 | 1.00 | 15.65 |
| ATOM | 2411 | CB | TYR | A | 315 | 0.768 | 24.264 | −18.957 | 1.00 | 15.82 |
| ATOM | 2412 | CG | TYR | A | 315 | 0.694 | 23.988 | −20.457 | 1.00 | 16.29 |
| ATOM | 2413 | CD1 | TYR | A | 315 | 1.824 | 24.124 | −21.265 | 1.00 | 17.01 |
| ATOM | 2414 | CE1 | TYR | A | 315 | 1.778 | 23.859 | −22.634 | 1.00 | 18.92 |
| ATOM | 2415 | CZ | TYR | A | 315 | 0.588 | 23.421 | −23.208 | 1.00 | 16.14 |
| ATOM | 2416 | OH | TYR | A | 315 | 0.557 | 23.164 | −24.577 | 1.00 | 16.95 |
| ATOM | 2417 | CE2 | TYR | A | 315 | −0.552 | 23.261 | −22.423 | 1.00 | 15.41 |
| ATOM | 2418 | CD2 | TYR | A | 315 | −0.492 | 23.539 | −21.044 | 1.00 | 14.48 |
| ATOM | 2419 | C | TYR | A | 315 | 0.489 | 26.777 | −19.107 | 1.00 | 15.56 |
| ATOM | 2420 | O | TYR | A | 315 | −0.688 | 26.888 | −18.748 | 1.00 | 16.36 |
| ATOM | 2421 | N | TYR | A | 316 | 1.072 | 27.645 | −19.944 | 1.00 | 16.61 |
| ATOM | 2422 | CA | TYR | A | 316 | 0.404 | 28.890 | −20.380 | 1.00 | 17.53 |
| ATOM | 2423 | CB | TYR | A | 316 | −0.778 | 28.603 | −21.337 | 1.00 | 18.31 |
| ATOM | 2424 | CG | TYR | A | 316 | −0.329 | 28.321 | −22.742 | 1.00 | 19.74 |
| ATOM | 2425 | CD1 | TYR | A | 316 | −0.071 | 27.026 | −23.169 | 1.00 | 18.95 |
| ATOM | 2426 | CE1 | TYR | A | 316 | 0.353 | 26.757 | −24.466 | 1.00 | 18.69 |
| ATOM | 2427 | CZ | TYR | A | 316 | 0.551 | 27.812 | −25.342 | 1.00 | 21.77 |
| ATOM | 2428 | OH | TYR | A | 316 | 1.002 | 27.557 | −26.617 | 1.00 | 23.75 |
| ATOM | 2429 | CE2 | TYR | A | 316 | 0.329 | 29.125 | −24.932 | 1.00 | 22.39 |
| ATOM | 2430 | CD2 | TYR | A | 316 | −0.111 | 29.369 | −23.639 | 1.00 | 21.90 |
| ATOM | 2431 | C | TYR | A | 316 | −0.037 | 29.730 | −19.173 | 1.00 | 17.87 |
| ATOM | 2432 | O | TYR | A | 316 | −0.968 | 30.517 | −19.266 | 1.00 | 17.06 |
| ATOM | 2433 | N | ASN | A | 317 | 0.689 | 29.555 | −18.066 | 1.00 | 18.34 |
| ATOM | 2434 | CA | ASN | A | 317 | 0.483 | 30.231 | −16.766 | 1.00 | 19.27 |
| ATOM | 2435 | CB | ASN | A | 317 | 0.106 | 31.699 | −16.921 | 1.00 | 20.01 |
| ATOM | 2436 | CG | ASN | A | 317 | 1.171 | 32.489 | −17.624 | 1.00 | 24.51 |
| ATOM | 2437 | OD1 | ASN | A | 317 | 2.363 | 32.384 | −17.305 | 1.00 | 29.46 |
| ATOM | 2438 | ND2 | ASN | A | 317 | 0.756 | 33.269 | −18.603 | 1.00 | 29.08 |
| ATOM | 2439 | C | ASN | A | 317 | −0.506 | 29.551 | −15.842 | 1.00 | 18.28 |
| ATOM | 2440 | O | ASN | A | 317 | −0.706 | 30.001 | −14.719 | 1.00 | 19.05 |
| ATOM | 2441 | N | GLY | A | 318 | −1.114 | 28.459 | −16.300 | 1.00 | 17.74 |
| ATOM | 2442 | CA | GLY | A | 318 | −2.086 | 27.721 | −15.475 | 1.00 | 15.34 |
| ATOM | 2443 | C | GLY | A | 318 | −3.458 | 28.356 | −15.550 | 1.00 | 15.47 |
| ATOM | 2444 | O | GLY | A | 318 | −3.700 | 29.390 | −14.932 | 1.00 | 15.75 |
| ATOM | 2445 | N | ASN | A | 319 | −4.369 | 27.733 | −16.306 | 1.00 | 13.16 |
| ATOM | 2446 | CA | ASN | A | 319 | −5.672 | 28.305 | −16.557 | 1.00 | 12.74 |
| ATOM | 2447 | CB | ASN | A | 319 | −5.693 | 28.883 | −17.980 | 1.00 | 12.31 |
| ATOM | 2448 | CG | ASN | A | 319 | −4.676 | 29.979 | −18.187 | 1.00 | 13.01 |
| ATOM | 2449 | OD1 | ASN | A | 319 | −4.832 | 31.117 | −17.699 | 1.00 | 14.18 |
| ATOM | 2450 | ND2 | ASN | A | 319 | −3.640 | 29.665 | −18.942 | 1.00 | 11.49 |
| ATOM | 2451 | C | ASN | A | 319 | −6.799 | 27.271 | −16.442 | 1.00 | 12.27 |
| ATOM | 2452 | O | ASN | A | 319 | −6.545 | 26.071 | −16.456 | 1.00 | 12.28 |
| ATOM | 2453 | N | PRO | A | 320 | −8.054 | 27.732 | −16.334 | 1.00 | 12.96 |
| ATOM | 2454 | CA | PRO | A | 320 | −9.113 | 26.759 | −16.472 | 1.00 | 12.58 |
| ATOM | 2455 | CB | PRO | A | 320 | −10.395 | 27.579 | −16.324 | 1.00 | 13.28 |
| ATOM | 2456 | CG | PRO | A | 320 | −10.007 | 29.011 | −16.183 | 1.00 | 14.34 |
| ATOM | 2457 | CD | PRO | A | 320 | −8.537 | 29.090 | −15.991 | 1.00 | 12.66 |
| ATOM | 2458 | C | PRO | A | 320 | −9.101 | 26.090 | −17.851 | 1.00 | 12.18 |
| ATOM | 2459 | O | PRO | A | 320 | −8.643 | 26.698 | −18.820 | 1.00 | 11.99 |
| ATOM | 2460 | N | TRP | A | 321 | −9.589 | 24.852 | −17.912 | 1.00 | 11.79 |
| ATOM | 2461 | CA | TRP | A | 321 | −9.739 | 24.116 | −19.154 | 1.00 | 11.97 |
| ATOM | 2462 | CB | TRP | A | 321 | −8.988 | 22.775 | −19.063 | 1.00 | 11.15 |
| ATOM | 2463 | CG | TRP | A | 321 | −7.469 | 22.900 | −19.200 | 1.00 | 12.16 |
| ATOM | 2464 | CD1 | TRP | A | 321 | −6.658 | 23.837 | −18.627 | 1.00 | 13.28 |
| ATOM | 2465 | NE1 | TRP | A | 321 | −5.347 | 23.636 | −19.016 | 1.00 | 13.24 |
| ATOM | 2466 | CE2 | TRP | A | 321 | −5.290 | 22.538 | −19.831 | 1.00 | 13.51 |
| ATOM | 2467 | CD2 | TRP | A | 321 | −6.617 | 22.054 | −19.978 | 1.00 | 12.68 |
| ATOM | 2468 | CE3 | TRP | A | 321 | −6.846 | 20.938 | −20.787 | 1.00 | 14.41 |
| ATOM | 2469 | CZ3 | TRP | A | 321 | −5.741 | 20.323 | −21.428 | 1.00 | 13.49 |
| ATOM | 2470 | CH2 | TRP | A | 321 | −4.436 | 20.819 | −21.250 | 1.00 | 13.43 |
| ATOM | 2471 | CZ2 | TRP | A | 321 | −4.193 | 21.948 | −20.479 | 1.00 | 14.69 |
| ATOM | 2472 | C | TRP | A | 321 | −11.202 | 23.797 | −19.342 | 1.00 | 11.99 |
| ATOM | 2473 | O | TRP | A | 321 | −11.875 | 23.448 | −18.388 | 1.00 | 11.51 |
| ATOM | 2474 | N | TYR | A | 322 | −11.696 | 23.896 | −20.579 | 1.00 | 11.47 |
| ATOM | 2475 | CA | TYR | A | 322 | −13.088 | 23.511 | −20.841 | 1.00 | 11.31 |
| ATOM | 2476 | CB | TYR | A | 322 | −13.433 | 23.691 | −22.322 | 1.00 | 12.14 |
| ATOM | 2477 | CG | TYR | A | 322 | −13.352 | 25.130 | −22.793 | 1.00 | 12.81 |
| ATOM | 2478 | CD1 | TYR | A | 322 | −12.260 | 25.574 | −23.509 | 1.00 | 11.43 |
| ATOM | 2479 | CE1 | TYR | A | 322 | −12.173 | 26.914 | −23.965 | 1.00 | 12.91 |
| ATOM | 2480 | CZ | TYR | A | 322 | −13.216 | 27.802 | −23.697 | 1.00 | 14.27 |
| ATOM | 2481 | OH | TYR | A | 322 | −13.127 | 29.104 | −24.146 | 1.00 | 15.10 |
| ATOM | 2482 | CE2 | TYR | A | 322 | −14.324 | 27.373 | −22.982 | 1.00 | 13.40 |
| ATOM | 2483 | CD2 | TYR | A | 322 | −14.378 | 26.031 | −22.522 | 1.00 | 11.58 |
| ATOM | 2484 | C | TYR | A | 322 | −13.367 | 22.082 | −20.433 | 1.00 | 11.31 |
| ATOM | 2485 | O | TYR | A | 322 | −14.380 | 21.795 | −19.771 | 1.00 | 11.08 |
| ATOM | 2486 | N | LEU | A | 323 | −12.480 | 21.169 | −20.814 | 1.00 | 10.96 |
| ATOM | 2487 | CA | LEU | A | 323 | −12.770 | 19.750 | −20.561 | 1.00 | 11.04 |
| ATOM | 2488 | CB | LEU | A | 323 | −11.787 | 18.844 | −21.315 | 1.00 | 11.26 |
| ATOM | 2489 | CG | LEU | A | 323 | −10.314 | 18.876 | −20.903 | 1.00 | 10.53 |
| ATOM | 2490 | CD1 | LEU | A | 323 | −10.074 | 17.902 | −19.745 | 1.00 | 14.57 |
| ATOM | 2491 | CD2 | LEU | A | 323 | −9.474 | 18.437 | −22.112 | 1.00 | 13.19 |
| ATOM | 2492 | C | LEU | A | 323 | −12.778 | 19.449 | −19.048 | 1.00 | 11.68 |
| ATOM | 2493 | O | LEU | A | 323 | −13.444 | 18.510 | −18.602 | 1.00 | 12.06 |
| ATOM | 2494 | N | ALA | A | 324 | −12.036 | 20.239 | −18.268 | 1.00 | 9.86 |
| ATOM | 2495 | CA | ALA | A | 324 | −11.969 | 20.017 | −16.812 | 1.00 | 10.09 |
| ATOM | 2496 | CB | ALA | A | 324 | −10.746 | 20.767 | −16.234 | 1.00 | 9.76 |
| ATOM | 2497 | C | ALA | A | 324 | −13.272 | 20.518 | −16.178 | 1.00 | 10.17 |
| ATOM | 2498 | O | ALA | A | 324 | −13.866 | 19.840 | −15.325 | 1.00 | 10.29 |
| ATOM | 2499 | N | THR | A | 325 | −13.758 | 21.662 | −16.665 | 1.00 | 9.84 |
| ATOM | 2500 | CA | THR | A | 325 | −15.000 | 22.267 | −16.172 | 1.00 | 11.14 |
| ATOM | 2501 | CB | THR | A | 325 | −15.102 | 23.765 | −16.623 | 1.00 | 12.15 |
| ATOM | 2502 | OG1 | THR | A | 325 | −14.002 | 24.498 | −16.063 | 1.00 | 13.16 |
| ATOM | 2503 | CG2 | THR | A | 325 | −16.402 | 24.411 | −16.152 | 1.00 | 11.83 |
| ATOM | 2504 | C | THR | A | 325 | −16.218 | 21.413 | −16.570 | 1.00 | 11.50 |
| ATOM | 2505 | O | THR | A | 325 | −17.086 | 21.126 | −15.727 | 1.00 | 10.79 |
| ATOM | 2506 | N | PHE | A | 326 | −16.234 | 20.925 | −17.816 | 1.00 | 10.79 |
| ATOM | 2507 | CA | PHE | A | 326 | −17.272 | 19.959 | −18.240 | 1.00 | 12.12 |
| ATOM | 2508 | CB | PHE | A | 326 | −17.194 | 19.652 | −19.746 | 1.00 | 12.14 |
| ATOM | 2509 | CG | PHE | A | 326 | −17.518 | 20.851 | −20.640 | 1.00 | 13.71 |
| ATOM | 2510 | CD1 | PHE | A | 326 | −16.777 | 21.077 | −21.804 | 1.00 | 15.21 |
| ATOM | 2511 | CE1 | PHE | A | 326 | −17.043 | 22.188 | −22.635 | 1.00 | 14.99 |
| ATOM | 2512 | CZ | PHE | A | 326 | −18.072 | 23.066 | −22.311 | 1.00 | 16.33 |
| ATOM | 2513 | CE2 | PHE | A | 326 | −18.851 | 22.832 | −21.160 | 1.00 | 20.12 |
| ATOM | 2514 | CD2 | PHE | A | 326 | −18.561 | 21.717 | −20.331 | 1.00 | 16.63 |
| ATOM | 2515 | C | PHE | A | 326 | −17.216 | 18.643 | −17.464 | 1.00 | 11.56 |
| ATOM | 2516 | O | PHE | A | 326 | −18.263 | 18.069 | −17.180 | 1.00 | 11.74 |
| ATOM | 2517 | N | ALA | A | 327 | −16.014 | 18.174 | −17.103 | 1.00 | 11.35 |
| ATOM | 2518 | CA | ALA | A | 327 | −15.889 | 16.909 | −16.346 | 1.00 | 11.41 |
| ATOM | 2519 | CB | ALA | A | 327 | −14.397 | 16.538 | −16.158 | 1.00 | 11.95 |
| ATOM | 2520 | C | ALA | A | 327 | −16.612 | 16.964 | −14.965 | 1.00 | 12.04 |
| ATOM | 2521 | O | ALA | A | 327 | −17.260 | 15.985 | −14.561 | 1.00 | 12.69 |
| ATOM | 2522 | N | ALA | A | 328 | −16.505 | 18.097 | −14.266 | 1.00 | 12.13 |
| ATOM | 2523 | CA | ALA | A | 328 | −17.207 | 18.293 | −12.985 | 1.00 | 12.24 |
| ATOM | 2524 | CB | ALA | A | 328 | −16.871 | 19.662 | −12.369 | 1.00 | 12.48 |
| ATOM | 2525 | C | ALA | A | 328 | −18.707 | 18.157 | −13.177 | 1.00 | 12.90 |
| ATOM | 2526 | O | ALA | A | 328 | −19.378 | 17.454 | −12.411 | 1.00 | 13.60 |
| ATOM | 2527 | N | ALA | A | 329 | −19.239 | 18.814 | −14.202 | 1.00 | 12.55 |
| ATOM | 2528 | CA | ALA | A | 329 | −20.669 | 18.682 | −14.504 | 1.00 | 12.31 |
| ATOM | 2529 | CB | ALA | A | 329 | −21.027 | 19.551 | −15.692 | 1.00 | 12.87 |
| ATOM | 2530 | C | ALA | A | 329 | −21.035 | 17.226 | −14.788 | 1.00 | 12.71 |
| ATOM | 2531 | O | ALA | A | 329 | −22.016 | 16.700 | −14.266 | 1.00 | 12.32 |
| ATOM | 2532 | N | GLU | A | 330 | −20.231 | 16.572 | −15.629 | 1.00 | 12.54 |
| ATOM | 2533 | CA | GLU | A | 330 | −20.500 | 15.187 | −16.003 | 1.00 | 12.84 |
| ATOM | 2534 | CB | GLU | A | 330 | −19.519 | 14.718 | −17.100 | 1.00 | 12.55 |
| ATOM | 2535 | CG | GLU | A | 330 | −19.850 | 13.303 | −17.626 | 1.00 | 13.80 |
| ATOM | 2536 | CD | GLU | A | 330 | −19.108 | 12.953 | −18.917 | 1.00 | 13.54 |
| ATOM | 2537 | OE1 | GLU | A | 330 | −18.650 | 13.889 | −19.604 | 1.00 | 12.29 |
| ATOM | 2538 | OE2 | GLU | A | 330 | −18.998 | 11.739 | −19.209 | 1.00 | 14.52 |
| ATOM | 2539 | C | GLU | A | 330 | −20.523 | 14.231 | −14.809 | 1.00 | 12.94 |
| ATOM | 2540 | O | GLU | A | 330 | −21.400 | 13.346 | −14.726 | 1.00 | 12.90 |
| ATOM | 2541 | N | GLN | A | 331 | −19.598 | 14.402 | −13.866 | 1.00 | 12.03 |
| ATOM | 2542 | CA | GLN | A | 331 | −19.589 | 13.502 | −12.726 | 1.00 | 12.38 |
| ATOM | 2543 | CB | GLN | A | 331 | −18.415 | 13.795 | −11.797 | 1.00 | 12.24 |
| ATOM | 2544 | CG | GLN | A | 331 | −18.357 | 12.759 | −10.670 | 1.00 | 13.61 |
| ATOM | 2545 | CD | GLN | A | 331 | −17.327 | 13.072 | −9.608 | 1.00 | 15.82 |
| ATOM | 2546 | OE1 | GLN | A | 331 | −16.263 | 13.617 | −9.895 | 1.00 | 15.39 |
| ATOM | 2547 | NE2 | GLN | A | 331 | −17.628 | 12.702 | −8.372 | 1.00 | 13.76 |
| ATOM | 2548 | C | GLN | A | 331 | −20.912 | 13.643 | −11.969 | 1.00 | 12.33 |
| ATOM | 2549 | O | GLN | A | 331 | −21.512 | 12.659 | −11.556 | 1.00 | 12.45 |
| ATOM | 2550 | N | LEU | A | 332 | −21.377 | 14.873 | −11.844 | 1.00 | 12.57 |
| ATOM | 2551 | CA | LEU | A | 332 | −22.628 | 15.138 | −11.134 | 1.00 | 13.59 |
| ATOM | 2552 | CB | LEU | A | 332 | −22.747 | 16.631 | −10.868 | 1.00 | 13.17 |
| ATOM | 2553 | CG | LEU | A | 332 | −21.681 | 17.142 | −9.867 | 1.00 | 16.56 |
| ATOM | 2554 | CD1 | LEU | A | 332 | −21.718 | 18.678 | −9.801 | 1.00 | 18.10 |
| ATOM | 2555 | CD2 | LEU | A | 332 | −21.851 | 16.476 | −8.492 | 1.00 | 19.47 |
| ATOM | 2556 | C | LEU | A | 332 | −23.861 | 14.600 | −11.864 | 1.00 | 13.57 |
| ATOM | 2557 | O | LEU | A | 332 | −24.770 | 14.053 | −11.239 | 1.00 | 13.24 |
| ATOM | 2558 | N | TYR | A | 333 | −23.909 | 14.766 | −13.179 | 1.00 | 13.90 |
| ATOM | 2559 | CA | TYR | A | 333 | −24.988 | 14.131 | −13.972 | 1.00 | 14.37 |
| ATOM | 2560 | CB | TYR | A | 333 | −24.901 | 14.523 | −15.468 | 1.00 | 14.48 |
| ATOM | 2561 | CG | TYR | A | 333 | −25.056 | 16.001 | −15.721 | 1.00 | 13.91 |
| ATOM | 2562 | CD1 | TYR | A | 333 | −26.086 | 16.738 | −15.118 | 1.00 | 14.64 |
| ATOM | 2563 | CE1 | TYR | A | 333 | −26.208 | 18.117 | −15.350 | 1.00 | 15.65 |
| ATOM | 2564 | CZ | TYR | A | 333 | −25.315 | 18.758 | −16.196 | 1.00 | 16.47 |
| ATOM | 2565 | OH | TYR | A | 333 | −25.431 | 20.101 | −16.442 | 1.00 | 17.22 |
| ATOM | 2566 | CE2 | TYR | A | 333 | −24.310 | 18.050 | −16.836 | 1.00 | 16.59 |
| ATOM | 2567 | CD2 | TYR | A | 333 | −24.192 | 16.669 | −16.601 | 1.00 | 11.27 |
| ATOM | 2568 | C | TYR | A | 333 | −25.022 | 12.613 | −13.843 | 1.00 | 15.03 |
| ATOM | 2569 | O | TYR | A | 333 | −26.108 | 12.012 | −13.824 | 1.00 | 14.78 |
| ATOM | 2570 | N | ASP | A | 334 | −23.836 | 11.998 | −13.807 | 1.00 | 14.06 |
| ATOM | 2571 | CA | ASP | A | 334 | −23.714 | 10.555 | −13.602 | 1.00 | 14.83 |
| ATOM | 2572 | CB | ASP | A | 334 | −22.239 | 10.114 | −13.714 | 1.00 | 13.83 |
| ATOM | 2573 | CG | ASP | A | 334 | −21.708 | 10.149 | −15.136 | 1.00 | 15.84 |
| ATOM | 2574 | OD1 | ASP | A | 334 | −22.495 | 10.373 | −16.081 | 1.00 | 13.95 |
| ATOM | 2575 | OD2 | ASP | A | 334 | −20.470 | 9.943 | −15.313 | 1.00 | 15.50 |
| ATOM | 2576 | C | ASP | A | 334 | −24.254 | 10.163 | −12.224 | 1.00 | 15.21 |
| ATOM | 2577 | O | ASP | A | 334 | −24.941 | 9.132 | −12.080 | 1.00 | 15.93 |
| ATOM | 2578 | N | ALA | A | 335 | −23.933 | 10.969 | −11.213 | 1.00 | 15.09 |
| ATOM | 2579 | CA | ALA | A | 335 | −24.454 | 10.735 | −9.855 | 1.00 | 16.00 |
| ATOM | 2580 | CB | ALA | A | 335 | −23.809 | 11.719 | −8.864 | 1.00 | 15.13 |
| ATOM | 2581 | C | ALA | A | 335 | −25.980 | 10.823 | −9.803 | 1.00 | 16.10 |
| ATOM | 2582 | O | ALA | A | 335 | −26.643 | 9.916 | −9.245 | 1.00 | 16.77 |
| ATOM | 2583 | N | ILE | A | 336 | −26.530 | 11.879 | −10.398 | 1.00 | 16.35 |
| ATOM | 2584 | CA | ILE | A | 336 | −27.987 | 12.087 | −10.470 | 1.00 | 18.39 |
| ATOM | 2585 | CB | ILE | A | 336 | −28.332 | 13.422 | −11.162 | 1.00 | 18.85 |
| ATOM | 2586 | CG1 | ILE | A | 336 | −27.891 | 14.596 | −10.279 | 1.00 | 19.14 |
| ATOM | 2587 | CD1 | ILE | A | 336 | −27.879 | 15.904 | −10.986 | 1.00 | 22.50 |
| ATOM | 2588 | CG2 | ILE | A | 336 | −29.839 | 13.539 | −11.506 | 1.00 | 20.14 |
| ATOM | 2589 | C | ILE | A | 336 | −28.681 | 10.902 | −11.156 | 1.00 | 18.83 |
| ATOM | 2590 | O | ILE | A | 336 | −29.707 | 10.404 | −10.675 | 1.00 | 18.06 |
| ATOM | 2591 | N | TYR | A | 337 | −28.102 | 10.443 | −12.267 | 1.00 | 18.50 |
| ATOM | 2592 | CA | TYR | A | 337 | −28.642 | 9.287 | −12.970 | 1.00 | 18.99 |
| ATOM | 2593 | CB | TYR | A | 337 | −27.753 | 8.908 | −14.169 | 1.00 | 19.80 |
| ATOM | 2594 | CG | TYR | A | 337 | −28.328 | 7.737 | −14.954 | 1.00 | 20.76 |
| ATOM | 2595 | CD1 | TYR | A | 337 | −27.988 | 6.429 | −14.620 | 1.00 | 20.95 |
| ATOM | 2596 | CE1 | TYR | A | 337 | −28.511 | 5.345 | −15.322 | 1.00 | 22.94 |
| ATOM | 2597 | CZ | TYR | A | 337 | −29.382 | 5.559 | −16.356 | 1.00 | 22.19 |
| ATOM | 2598 | OH | TYR | A | 337 | −29.877 | 4.447 | −17.018 | 1.00 | 24.87 |
| ATOM | 2599 | CE2 | TYR | A | 337 | −29.752 | 6.845 | −16.721 | 1.00 | 22.31 |
| ATOM | 2600 | CD2 | TYR | A | 337 | −29.220 | 7.942 | −16.009 | 1.00 | 21.58 |
| ATOM | 2601 | C | TYR | A | 337 | −28.839 | 8.083 | −12.057 | 1.00 | 18.60 |
| ATOM | 2602 | O | TYR | A | 337 | −29.918 | 7.476 | −12.041 | 1.00 | 18.61 |
| ATOM | 2603 | N | VAL | A | 338 | −27.802 | 7.737 | −11.297 | 1.00 | 18.67 |
| ATOM | 2604 | CA | VAL | A | 338 | −27.837 | 6.573 | −10.406 | 1.00 | 18.90 |
| ATOM | 2605 | CB | VAL | A | 338 | −26.424 | 6.195 | −9.919 | 1.00 | 18.99 |
| ATOM | 2606 | CG1 | VAL | A | 338 | −26.462 | 5.121 | −8.820 | 1.00 | 19.71 |
| ATOM | 2607 | CG2 | VAL | A | 338 | −25.600 | 5.698 | −11.111 | 1.00 | 18.75 |
| ATOM | 2608 | C | VAL | A | 338 | −28.810 | 6.788 | −9.234 | 1.00 | 19.41 |
| ATOM | 2609 | O | VAL | A | 338 | −29.565 | 5.871 | −8.869 | 1.00 | 19.45 |
| ATOM | 2610 | N | TRP | A | 339 | −28.797 | 7.987 | −8.654 | 1.00 | 19.81 |
| ATOM | 2611 | CA | TRP | A | 339 | −29.743 | 8.290 | −7.559 | 1.00 | 20.46 |
| ATOM | 2612 | CB | TRP | A | 339 | −29.514 | 9.705 | −7.029 | 1.00 | 20.35 |
| ATOM | 2613 | CG | TRP | A | 339 | −28.222 | 9.830 | −6.329 | 1.00 | 18.64 |
| ATOM | 2614 | CD1 | TRP | A | 339 | −27.540 | 8.846 | −5.676 | 1.00 | 16.51 |
| ATOM | 2615 | NE1 | TRP | A | 339 | −26.391 | 9.359 | −5.126 | 1.00 | 17.81 |
| ATOM | 2616 | CE2 | TRP | A | 339 | −26.312 | 10.693 | −5.423 | 1.00 | 17.12 |
| ATOM | 2617 | CD2 | TRP | A | 339 | −27.452 | 11.025 | −6.183 | 1.00 | 17.64 |
| ATOM | 2618 | CE3 | TRP | A | 339 | −27.624 | 12.343 | −6.614 | 1.00 | 17.59 |
| ATOM | 2619 | CZ3 | TRP | A | 339 | −26.637 | 13.283 | −6.284 | 1.00 | 19.24 |
| ATOM | 2620 | CH2 | TRP | A | 339 | −25.510 | 12.912 | −5.520 | 1.00 | 18.24 |
| ATOM | 2621 | CZ2 | TRP | A | 339 | −25.320 | 11.626 | −5.103 | 1.00 | 18.45 |
| ATOM | 2622 | C | TRP | A | 339 | −31.201 | 8.108 | −7.997 | 1.00 | 21.83 |
| ATOM | 2623 | O | TRP | A | 339 | −31.981 | 7.478 | −7.274 | 1.00 | 22.01 |
| ATOM | 2624 | N | LYS | A | 340 | −31.549 | 8.646 | −9.168 | 1.00 | 22.85 |
| ATOM | 2625 | CA | LYS | A | 340 | −32.904 | 8.541 | −9.721 | 1.00 | 25.61 |
| ATOM | 2626 | CB | LYS | A | 340 | −33.066 | 9.411 | −10.967 | 1.00 | 25.52 |
| ATOM | 2627 | CG | LYS | A | 340 | −33.174 | 10.905 | −10.689 | 1.00 | 28.19 |
| ATOM | 2628 | CD | LYS | A | 340 | −33.227 | 11.692 | −11.991 | 1.00 | 34.04 |
| ATOM | 2629 | CE | LYS | A | 340 | −33.966 | 13.011 | −11.805 | 1.00 | 38.04 |
| ATOM | 2630 | NZ | LYS | A | 340 | −33.868 | 13.876 | −13.017 | 1.00 | 41.83 |
| ATOM | 2631 | C | LYS | A | 340 | −33.276 | 7.108 | −10.062 | 1.00 | 27.14 |
| ATOM | 2632 | O | LYS | A | 340 | −34.413 | 6.686 | −9.830 | 1.00 | 27.56 |
| ATOM | 2633 | N | LYS | A | 341 | −32.317 | 6.358 | −10.604 | 1.00 | 28.13 |
| ATOM | 2634 | CA | LYS | A | 341 | −32.552 | 4.975 | −11.018 | 1.00 | 30.18 |
| ATOM | 2635 | CB | LYS | A | 341 | −31.358 | 4.428 | −11.800 | 1.00 | 29.83 |
| ATOM | 2636 | CG | LYS | A | 341 | −31.688 | 3.173 | −12.624 | 1.00 | 33.04 |
| ATOM | 2637 | CD | LYS | A | 341 | −30.472 | 2.624 | −13.395 | 1.00 | 33.62 |
| ATOM | 2638 | CE | LYS | A | 341 | −29.652 | 1.592 | −12.588 | 1.00 | 38.22 |
| ATOM | 2639 | NZ | LYS | A | 341 | −28.691 | 2.188 | −11.573 | 1.00 | 40.88 |
| ATOM | 2640 | C | LYS | A | 341 | −32.816 | 4.081 | −9.817 | 1.00 | 30.19 |
| ATOM | 2641 | O | LYS | A | 341 | −33.744 | 3.260 | −9.837 | 1.00 | 30.15 |
| ATOM | 2642 | N | THR | A | 342 | −31.999 | 4.246 | −8.777 | 1.00 | 29.52 |
| ATOM | 2643 | CA | THR | A | 342 | −32.074 | 3.400 | −7.595 | 1.00 | 29.75 |
| ATOM | 2644 | CB | THR | A | 342 | −30.687 | 3.221 | −6.916 | 1.00 | 29.68 |
| ATOM | 2645 | OG1 | THR | A | 342 | −30.254 | 4.458 | −6.333 | 1.00 | 32.01 |
| ATOM | 2646 | CG2 | THR | A | 342 | −29.628 | 2.735 | −7.929 | 1.00 | 31.40 |
| ATOM | 2647 | C | THR | A | 342 | −33.129 | 3.901 | −6.596 | 1.00 | 29.01 |
| ATOM | 2648 | O | THR | A | 342 | −33.572 | 3.148 | −5.734 | 1.00 | 29.92 |
| ATOM | 2649 | N | GLY | A | 343 | −33.534 | 5.158 | −6.732 | 1.00 | 28.20 |
| ATOM | 2650 | CA | GLY | A | 343 | −34.537 | 5.782 | −5.862 | 1.00 | 28.30 |
| ATOM | 2651 | C | GLY | A | 343 | −34.068 | 6.045 | −4.438 | 1.00 | 27.91 |
| ATOM | 2652 | O | GLY | A | 343 | −34.887 | 6.133 | −3.519 | 1.00 | 28.16 |
| ATOM | 2653 | N | SER | A | 344 | −32.760 | 6.226 | −4.260 | 1.00 | 27.22 |
| ATOM | 2654 | CA | SER | A | 344 | −32.142 | 6.306 | −2.939 | 1.00 | 26.60 |
| ATOM | 2655 | CB | SER | A | 344 | −31.870 | 4.880 | −2.462 | 1.00 | 27.28 |
| ATOM | 2656 | OG | SER | A | 344 | −31.354 | 4.855 | −1.161 | 1.00 | 29.50 |
| ATOM | 2657 | C | SER | A | 344 | −30.823 | 7.107 | −2.979 | 1.00 | 26.02 |
| ATOM | 2658 | O | SER | A | 344 | −30.068 | 6.992 | −3.944 | 1.00 | 25.80 |
| ATOM | 2659 | N | ILE | A | 345 | −30.557 | 7.900 | −1.936 | 1.00 | 24.20 |
| ATOM | 2660 | CA | ILE | A | 345 | −29.295 | 8.641 | −1.770 | 1.00 | 23.04 |
| ATOM | 2661 | CB | ILE | A | 345 | −29.477 | 10.171 | −1.954 | 1.00 | 23.03 |
| ATOM | 2662 | CG1 | ILE | A | 345 | −30.021 | 10.474 | −3.340 | 1.00 | 22.44 |
| ATOM | 2663 | CD1 | ILE | A | 345 | −30.399 | 11.918 | −3.599 | 1.00 | 23.24 |
| ATOM | 2664 | CG2 | ILE | A | 345 | −28.138 | 10.918 | −1.670 | 1.00 | 22.00 |
| ATOM | 2665 | C | ILE | A | 345 | −28.726 | 8.415 | −0.378 | 1.00 | 23.13 |
| ATOM | 2666 | O | ILE | A | 345 | −29.392 | 8.684 | 0.623 | 1.00 | 23.57 |
| ATOM | 2667 | N | THR | A | 346 | −27.490 | 7.943 | −0.307 | 1.00 | 22.23 |
| ATOM | 2668 | CA | THR | A | 346 | −26.820 | 7.765 | 0.963 | 1.00 | 23.25 |
| ATOM | 2669 | CB | THR | A | 346 | −26.246 | 6.338 | 1.101 | 1.00 | 23.78 |
| ATOM | 2670 | OG1 | THR | A | 346 | −27.327 | 5.396 | 1.020 | 1.00 | 27.42 |
| ATOM | 2671 | CG2 | THR | A | 346 | −25.507 | 6.129 | 2.443 | 1.00 | 24.74 |
| ATOM | 2672 | C | THR | A | 346 | −25.753 | 8.849 | 1.138 | 1.00 | 23.03 |
| ATOM | 2673 | O | THR | A | 346 | −24.916 | 9.067 | 0.260 | 1.00 | 23.47 |
| ATOM | 2674 | N | VAL | A | 347 | −25.848 | 9.561 | 2.255 | 1.00 | 21.33 |
| ATOM | 2675 | CA | VAL | A | 347 | −24.845 | 10.537 | 2.674 | 1.00 | 20.11 |
| ATOM | 2676 | CB | VAL | A | 347 | −25.522 | 11.844 | 3.212 | 1.00 | 19.06 |
| ATOM | 2677 | CG1 | VAL | A | 347 | −24.489 | 12.834 | 3.700 | 1.00 | 19.66 |
| ATOM | 2678 | CG2 | VAL | A | 347 | −26.418 | 12.465 | 2.137 | 1.00 | 20.32 |
| ATOM | 2679 | C | VAL | A | 347 | −24.066 | 9.865 | 3.785 | 1.00 | 20.14 |
| ATOM | 2680 | O | VAL | A | 347 | −24.667 | 9.340 | 4.728 | 1.00 | 19.79 |
| ATOM | 2681 | N | THR | A | 348 | −22.734 | 9.878 | 3.671 | 1.00 | 19.85 |
| ATOM | 2682 | CA | THR | A | 348 | −21.851 | 9.274 | 4.660 | 1.00 | 19.92 |
| ATOM | 2683 | CB | THR | A | 348 | −20.965 | 8.185 | 4.018 | 1.00 | 19.82 |
| ATOM | 2684 | OG1 | THR | A | 348 | −19.921 | 8.815 | 3.277 | 1.00 | 20.35 |
| ATOM | 2685 | CG2 | THR | A | 348 | −21.785 | 7.278 | 3.092 | 1.00 | 21.67 |
| ATOM | 2686 | C | THR | A | 348 | −20.964 | 10.354 | 5.256 | 1.00 | 19.60 |
| ATOM | 2687 | O | THR | A | 348 | −20.961 | 11.484 | 4.760 | 1.00 | 19.51 |
| ATOM | 2688 | N | ALA | A | 349 | −20.191 | 10.006 | 6.292 | 1.00 | 19.18 |
| ATOM | 2689 | CA | ALA | A | 349 | −19.243 | 10.932 | 6.885 | 1.00 | 20.02 |
| ATOM | 2690 | CB | ALA | A | 349 | −18.494 | 10.275 | 8.044 | 1.00 | 20.61 |
| ATOM | 2691 | C | ALA | A | 349 | −18.240 | 11.466 | 5.842 | 1.00 | 19.79 |
| ATOM | 2692 | O | ALA | A | 349 | −17.756 | 12.601 | 5.947 | 1.00 | 20.15 |
| ATOM | 2693 | N | THR | A | 350 | −17.906 | 10.619 | 4.873 | 1.00 | 18.87 |
| ATOM | 2694 | CA | THR | A | 350 | −16.911 | 10.971 | 3.850 | 1.00 | 18.30 |
| ATOM | 2695 | CB | THR | A | 350 | −16.435 | 9.717 | 3.093 | 1.00 | 18.67 |
| ATOM | 2696 | OG1 | THR | A | 350 | −15.780 | 8.850 | 4.027 | 1.00 | 19.82 |
| ATOM | 2697 | CG2 | THR | A | 350 | −15.426 | 10.097 | 1.974 | 1.00 | 17.73 |
| ATOM | 2698 | C | THR | A | 350 | −17.463 | 12.003 | 2.871 | 1.00 | 17.30 |
| ATOM | 2699 | O | THR | A | 350 | −16.747 | 12.930 | 2.487 | 1.00 | 17.99 |
| ATOM | 2700 | N | SER | A | 351 | −18.716 | 11.847 | 2.467 | 1.00 | 16.16 |
| ATOM | 2701 | CA | SER | A | 351 | −19.316 | 12.803 | 1.517 | 1.00 | 15.78 |
| ATOM | 2702 | CB | SER | A | 351 | −20.214 | 12.076 | 0.512 | 1.00 | 15.85 |
| ATOM | 2703 | OG | SER | A | 351 | −21.280 | 11.412 | 1.156 | 1.00 | 17.14 |
| ATOM | 2704 | C | SER | A | 351 | −20.087 | 13.941 | 2.193 | 1.00 | 15.63 |
| ATOM | 2705 | O | SER | A | 351 | −20.736 | 14.743 | 1.524 | 1.00 | 13.62 |
| ATOM | 2706 | N | LEU | A | 352 | −20.048 | 14.006 | 3.527 | 1.00 | 15.09 |
| ATOM | 2707 | CA | LEU | A | 352 | −20.901 | 14.985 | 4.212 | 1.00 | 16.29 |
| ATOM | 2708 | CB | LEU | A | 352 | −20.759 | 14.851 | 5.736 | 1.00 | 16.64 |
| ATOM | 2709 | CG | LEU | A | 352 | −21.713 | 15.734 | 6.570 | 1.00 | 17.22 |
| ATOM | 2710 | CD1 | LEU | A | 352 | −23.138 | 15.281 | 6.418 | 1.00 | 19.06 |
| ATOM | 2711 | CD2 | LEU | A | 352 | −21.263 | 15.636 | 8.032 | 1.00 | 20.02 |
| ATOM | 2712 | C | LEU | A | 352 | −20.592 | 16.427 | 3.787 | 1.00 | 16.09 |
| ATOM | 2713 | O | LEU | A | 352 | −21.499 | 17.219 | 3.601 | 1.00 | 16.35 |
| ATOM | 2714 | N | ALA | A | 353 | −19.311 | 16.763 | 3.643 | 1.00 | 16.19 |
| ATOM | 2715 | CA | ALA | A | 353 | −18.933 | 18.148 | 3.354 | 1.00 | 15.74 |
| ATOM | 2716 | CB | ALA | A | 353 | −17.460 | 18.314 | 3.424 | 1.00 | 16.30 |
| ATOM | 2717 | C | ALA | A | 353 | −19.459 | 18.544 | 1.972 | 1.00 | 16.11 |
| ATOM | 2718 | O | ALA | A | 353 | −19.957 | 19.668 | 1.781 | 1.00 | 15.44 |
| ATOM | 2719 | N | PHE | A | 354 | −19.367 | 17.607 | 1.021 | 1.00 | 16.10 |
| ATOM | 2720 | CA | PHE | A | 354 | −19.885 | 17.849 | −0.325 | 1.00 | 15.25 |
| ATOM | 2721 | CB | PHE | A | 354 | −19.718 | 16.618 | −1.220 | 1.00 | 16.59 |
| ATOM | 2722 | CG | PHE | A | 354 | −20.497 | 16.707 | −2.500 | 1.00 | 15.45 |
| ATOM | 2723 | CD1 | PHE | A | 354 | −19.959 | 17.375 | −3.603 | 1.00 | 16.95 |
| ATOM | 2724 | CE1 | PHE | A | 354 | −20.664 | 17.489 | −4.793 | 1.00 | 17.06 |
| ATOM | 2725 | CZ | PHE | A | 354 | −21.956 | 16.953 | −4.888 | 1.00 | 16.75 |
| ATOM | 2726 | CE2 | PHE | A | 354 | −22.517 | 16.276 | −3.791 | 1.00 | 16.91 |
| ATOM | 2727 | CD2 | PHE | A | 354 | −21.778 | 16.160 | −2.594 | 1.00 | 17.48 |
| ATOM | 2728 | C | PHE | A | 354 | −21.374 | 18.188 | −0.226 | 1.00 | 15.56 |
| ATOM | 2729 | O | PHE | A | 354 | −21.815 | 19.183 | −0.797 | 1.00 | 15.08 |
| ATOM | 2730 | N | PHE | A | 355 | −22.140 | 17.347 | 0.474 | 1.00 | 14.54 |
| ATOM | 2731 | CA | PHE | A | 355 | −23.588 | 17.544 | 0.517 | 1.00 | 15.29 |
| ATOM | 2732 | CB | PHE | A | 355 | −24.295 | 16.319 | 1.078 | 1.00 | 15.61 |
| ATOM | 2733 | CG | PHE | A | 355 | −24.386 | 15.176 | 0.112 | 1.00 | 15.51 |
| ATOM | 2734 | CD1 | PHE | A | 355 | −25.306 | 15.205 | −0.945 | 1.00 | 14.91 |
| ATOM | 2735 | CE1 | PHE | A | 355 | −25.404 | 14.131 | −1.832 | 1.00 | 16.52 |
| ATOM | 2736 | CZ | PHE | A | 355 | −24.567 | 13.033 | −1.676 | 1.00 | 16.12 |
| ATOM | 2737 | CE2 | PHE | A | 355 | −23.648 | 12.994 | −0.628 | 1.00 | 15.62 |
| ATOM | 2738 | CD2 | PHE | A | 355 | −23.562 | 14.071 | 0.255 | 1.00 | 13.62 |
| ATOM | 2739 | C | PHE | A | 355 | −23.988 | 18.789 | 1.303 | 1.00 | 15.19 |
| ATOM | 2740 | O | PHE | A | 355 | −24.920 | 19.477 | 0.902 | 1.00 | 15.69 |
| ATOM | 2741 | N | GLN | A | 356 | −23.283 | 19.084 | 2.398 | 1.00 | 15.78 |
| ATOM | 2742 | CA | GLN | A | 356 | −23.679 | 20.216 | 3.257 | 1.00 | 16.16 |
| ATOM | 2743 | CB | GLN | A | 356 | −22.987 | 20.165 | 4.627 | 1.00 | 16.84 |
| ATOM | 2744 | CG | GLN | A | 356 | −23.564 | 19.115 | 5.579 | 1.00 | 17.13 |
| ATOM | 2745 | CD | GLN | A | 356 | −22.907 | 19.170 | 6.973 | 1.00 | 18.06 |
| ATOM | 2746 | OE1 | GLN | A | 356 | −21.808 | 19.707 | 7.138 | 1.00 | 20.29 |
| ATOM | 2747 | NE2 | GLN | A | 356 | −23.569 | 18.589 | 7.965 | 1.00 | 20.95 |
| ATOM | 2748 | C | GLN | A | 356 | −23.444 | 21.546 | 2.584 | 1.00 | 16.08 |
| ATOM | 2749 | O | GLN | A | 356 | −24.142 | 22.507 | 2.868 | 1.00 | 15.59 |
| ATOM | 2750 | N | GLU | A | 357 | −22.482 | 21.599 | 1.661 | 1.00 | 16.07 |
| ATOM | 2751 | CA | GLU | A | 357 | −22.261 | 22.808 | 0.884 | 1.00 | 16.41 |
| ATOM | 2752 | CB | GLU | A | 357 | −20.994 | 22.683 | 0.005 | 1.00 | 16.33 |
| ATOM | 2753 | CG | GLU | A | 357 | −20.671 | 23.942 | −0.770 | 1.00 | 15.64 |
| ATOM | 2754 | CD | GLU | A | 357 | −19.326 | 23.894 | −1.516 | 1.00 | 17.67 |
| ATOM | 2755 | OE1 | GLU | A | 357 | −18.931 | 24.947 | −2.066 | 1.00 | 19.03 |
| ATOM | 2756 | OE2 | GLU | A | 357 | −18.685 | 22.822 | −1.575 | 1.00 | 14.75 |
| ATOM | 2757 | C | GLU | A | 357 | −23.492 | 23.105 | 0.019 | 1.00 | 15.84 |
| ATOM | 2758 | O | GLU | A | 357 | −23.786 | 24.237 | −0.224 | 1.00 | 18.07 |
| ATOM | 2759 | N | LEU | A | 358 | −24.213 | 22.084 | −0.420 | 1.00 | 14.72 |
| ATOM | 2760 | CA | LEU | A | 358 | −25.364 | 22.251 | −1.310 | 1.00 | 15.75 |
| ATOM | 2761 | CB | LEU | A | 358 | −25.368 | 21.147 | −2.369 | 1.00 | 16.49 |
| ATOM | 2762 | CG | LEU | A | 358 | −24.057 | 21.100 | −3.168 | 1.00 | 16.81 |
| ATOM | 2763 | CD1 | LEU | A | 358 | −24.087 | 19.977 | −4.182 | 1.00 | 19.73 |
| ATOM | 2764 | CD2 | LEU | A | 358 | −23.775 | 22.465 | −3.846 | 1.00 | 19.34 |
| ATOM | 2765 | C | LEU | A | 358 | −26.708 | 22.251 | −0.582 | 1.00 | 15.36 |
| ATOM | 2766 | O | LEU | A | 358 | −27.656 | 22.911 | −1.028 | 1.00 | 14.78 |
| ATOM | 2767 | N | VAL | A | 359 | −26.786 | 21.511 | 0.520 | 1.00 | 15.34 |
| ATOM | 2768 | CA | VAL | A | 359 | −28.001 | 21.404 | 1.321 | 1.00 | 15.00 |
| ATOM | 2769 | CB | VAL | A | 359 | −28.691 | 20.006 | 1.154 | 1.00 | 15.90 |
| ATOM | 2770 | CG1 | VAL | A | 359 | −29.999 | 19.917 | 1.962 | 1.00 | 15.16 |
| ATOM | 2771 | CG2 | VAL | A | 359 | −28.967 | 19.685 | −0.348 | 1.00 | 16.26 |
| ATOM | 2772 | C | VAL | A | 359 | −27.531 | 21.624 | 2.775 | 1.00 | 14.72 |
| ATOM | 2773 | O | VAL | A | 359 | −27.192 | 20.653 | 3.500 | 1.00 | 14.22 |
| ATOM | 2774 | N | PRO | A | 360 | −27.467 | 22.893 | 3.193 | 1.00 | 14.98 |
| ATOM | 2775 | CA | PRO | A | 360 | −26.937 | 23.179 | 4.539 | 1.00 | 15.45 |
| ATOM | 2776 | CB | PRO | A | 360 | −27.150 | 24.700 | 4.700 | 1.00 | 15.45 |
| ATOM | 2777 | CG | PRO | A | 360 | −27.188 | 25.219 | 3.274 | 1.00 | 16.15 |
| ATOM | 2778 | CD | PRO | A | 360 | −27.854 | 24.127 | 2.471 | 1.00 | 14.54 |
| ATOM | 2779 | C | PRO | A | 360 | −27.692 | 22.385 | 5.611 | 1.00 | 15.31 |
| ATOM | 2780 | O | PRO | A | 360 | −28.918 | 22.262 | 5.555 | 1.00 | 15.07 |
| ATOM | 2781 | N | GLY | A | 361 | −26.936 | 21.842 | 6.560 | 1.00 | 15.94 |
| ATOM | 2782 | CA | GLY | A | 361 | −27.512 | 21.143 | 7.709 | 1.00 | 16.53 |
| ATOM | 2783 | C | GLY | A | 361 | −27.870 | 19.680 | 7.488 | 1.00 | 17.03 |
| ATOM | 2784 | O | GLY | A | 361 | −28.268 | 18.997 | 8.429 | 1.00 | 17.68 |
| ATOM | 2785 | N | VAL | A | 362 | −27.762 | 19.176 | 6.261 | 1.00 | 16.13 |
| ATOM | 2786 | CA | VAL | A | 362 | −28.163 | 17.769 | 6.037 | 1.00 | 16.72 |
| ATOM | 2787 | CB | VAL | A | 362 | −28.217 | 17.416 | 4.525 | 1.00 | 16.47 |
| ATOM | 2788 | CG1 | VAL | A | 362 | −26.808 | 17.311 | 3.947 | 1.00 | 16.75 |
| ATOM | 2789 | CG2 | VAL | A | 362 | −29.054 | 16.142 | 4.280 | 1.00 | 17.21 |
| ATOM | 2790 | C | VAL | A | 362 | −27.208 | 16.849 | 6.811 | 1.00 | 17.24 |
| ATOM | 2791 | O | VAL | A | 362 | −26.044 | 17.187 | 7.006 | 1.00 | 17.07 |
| ATOM | 2792 | N | THR | A | 363 | −27.695 | 15.703 | 7.274 | 1.00 | 18.15 |
| ATOM | 2793 | CA | THR | A | 363 | −26.821 | 14.789 | 8.025 | 1.00 | 19.86 |
| ATOM | 2794 | CB | THR | A | 363 | −27.388 | 14.459 | 9.405 | 1.00 | 20.59 |
| ATOM | 2795 | OG1 | THR | A | 363 | −28.634 | 13.776 | 9.217 | 1.00 | 22.85 |
| ATOM | 2796 | CG2 | THR | A | 363 | −27.610 | 15.742 | 10.182 | 1.00 | 22.71 |
| ATOM | 2797 | C | THR | A | 363 | −26.660 | 13.476 | 7.310 | 1.00 | 19.18 |
| ATOM | 2798 | O | THR | A | 363 | −27.398 | 13.184 | 6.371 | 1.00 | 18.87 |
| ATOM | 2799 | N | ALA | A | 364 | −25.697 | 12.679 | 7.769 | 1.00 | 19.67 |
| ATOM | 2800 | CA | ALA | A | 364 | −25.495 | 11.342 | 7.222 | 1.00 | 20.19 |
| ATOM | 2801 | CB | ALA | A | 364 | −24.361 | 10.637 | 7.930 | 1.00 | 20.38 |
| ATOM | 2802 | C | ALA | A | 364 | −26.783 | 10.541 | 7.343 | 1.00 | 21.17 |
| ATOM | 2803 | O | ALA | A | 364 | −27.551 | 10.720 | 8.293 | 1.00 | 21.92 |
| ATOM | 2804 | N | GLY | A | 365 | −27.041 | 9.687 | 6.360 | 1.00 | 21.67 |
| ATOM | 2805 | CA | GLY | A | 365 | −28.207 | 8.823 | 6.371 | 1.00 | 22.35 |
| ATOM | 2806 | C | GLY | A | 365 | −28.584 | 8.415 | 4.968 | 1.00 | 23.66 |
| ATOM | 2807 | O | GLY | A | 365 | −27.924 | 8.804 | 3.991 | 1.00 | 23.66 |
| ATOM | 2808 | N | THR | A | 366 | −29.639 | 7.615 | 4.862 | 1.00 | 23.70 |
| ATOM | 2809 | CA | THR | A | 366 | −30.148 | 7.183 | 3.582 | 1.00 | 24.56 |
| ATOM | 2810 | CB | THR | A | 366 | −30.188 | 5.644 | 3.491 | 1.00 | 25.67 |
| ATOM | 2811 | OG1 | THR | A | 366 | −28.849 | 5.143 | 3.649 | 1.00 | 27.09 |
| ATOM | 2812 | CG2 | THR | A | 366 | −30.715 | 5.216 | 2.159 | 1.00 | 25.47 |
| ATOM | 2813 | C | THR | A | 366 | −31.520 | 7.769 | 3.344 | 1.00 | 25.15 |
| ATOM | 2814 | O | THR | A | 366 | −32.427 | 7.612 | 4.177 | 1.00 | 25.01 |
| ATOM | 2815 | N | TYR | A | 367 | −31.668 | 8.447 | 2.210 | 1.00 | 24.20 |
| ATOM | 2816 | CA | TYR | A | 367 | −32.900 | 9.146 | 1.883 | 1.00 | 24.30 |
| ATOM | 2817 | CB | TYR | A | 367 | −32.616 | 10.648 | 1.701 | 1.00 | 23.51 |
| ATOM | 2818 | CG | TYR | A | 367 | −31.924 | 11.238 | 2.907 | 1.00 | 22.68 |
| ATOM | 2819 | CD1 | TYR | A | 367 | −32.639 | 11.506 | 4.078 | 1.00 | 21.99 |
| ATOM | 2820 | CE1 | TYR | A | 367 | −32.012 | 12.019 | 5.199 | 1.00 | 20.27 |
| ATOM | 2821 | CZ | TYR | A | 367 | −30.650 | 12.263 | 5.176 | 1.00 | 22.67 |
| ATOM | 2822 | OH | TYR | A | 367 | −30.036 | 12.789 | 6.287 | 1.00 | 21.24 |
| ATOM | 2823 | CE2 | TYR | A | 367 | −29.897 | 11.994 | 4.023 | 1.00 | 20.71 |
| ATOM | 2824 | CD2 | TYR | A | 367 | −30.541 | 11.479 | 2.904 | 1.00 | 20.37 |
| ATOM | 2825 | C | TYR | A | 367 | −33.531 | 8.542 | 0.641 | 1.00 | 25.39 |
| ATOM | 2826 | O | TYR | A | 367 | −32.900 | 8.456 | −0.415 | 1.00 | 24.86 |
| ATOM | 2827 | N | SER | A | 368 | −34.782 | 8.109 | 0.758 | 1.00 | 25.82 |
| ATOM | 2828 | CA | SER | A | 368 | −35.416 | 7.454 | −0.374 | 1.00 | 27.29 |
| ATOM | 2829 | CB | SER | A | 368 | −36.339 | 6.332 | 0.107 | 1.00 | 27.90 |
| ATOM | 2830 | OG | SER | A | 368 | −37.519 | 6.895 | 0.634 | 1.00 | 30.69 |
| ATOM | 2831 | C | SER | A | 368 | −36.171 | 8.466 | −1.218 | 1.00 | 27.68 |
| ATOM | 2832 | O | SER | A | 368 | −36.361 | 9.612 | −0.805 | 1.00 | 26.96 |
| ATOM | 2833 | N | SER | A | 369 | −36.629 | 8.025 | −2.388 | 1.00 | 28.98 |
| ATOM | 2834 | CA | SER | A | 369 | −37.260 | 8.908 | −3.367 | 1.00 | 30.52 |
| ATOM | 2835 | CB | SER | A | 369 | −37.520 | 8.167 | −4.681 | 1.00 | 30.90 |
| ATOM | 2836 | OG | SER | A | 369 | −38.269 | 6.983 | −4.452 | 1.00 | 32.57 |
| ATOM | 2837 | C | SER | A | 369 | −38.536 | 9.583 | −2.871 | 1.00 | 31.50 |
| ATOM | 2838 | O | SER | A | 369 | −38.954 | 10.572 | −3.442 | 1.00 | 32.33 |
| ATOM | 2839 | N | SER | A | 370 | −39.150 | 9.067 | −1.809 | 1.00 | 32.18 |
| ATOM | 2840 | CA | SER | A | 370 | −40.365 | 9.712 | −1.279 | 1.00 | 32.96 |
| ATOM | 2841 | CB | SER | A | 370 | −41.313 | 8.692 | −0.624 | 1.00 | 33.18 |
| ATOM | 2842 | OG | SER | A | 370 | −40.610 | 7.847 | 0.273 | 1.00 | 34.18 |
| ATOM | 2843 | C | SER | A | 370 | −40.049 | 10.864 | −0.323 | 1.00 | 32.37 |
| ATOM | 2844 | O | SER | A | 370 | −40.901 | 11.729 | −0.078 | 1.00 | 33.26 |
| ATOM | 2845 | N | SER | A | 371 | −38.825 | 10.893 | 0.197 | 1.00 | 31.13 |
| ATOM | 2846 | CA | SER | A | 371 | −38.443 | 11.911 | 1.174 | 1.00 | 30.04 |
| ATOM | 2847 | CB | SER | A | 371 | −37.180 | 11.487 | 1.912 | 1.00 | 29.83 |
| ATOM | 2848 | OG | SER | A | 371 | −36.046 | 11.714 | 1.100 | 1.00 | 30.43 |
| ATOM | 2849 | C | SER | A | 371 | −38.247 | 13.295 | 0.553 | 1.00 | 29.13 |
| ATOM | 2850 | O | SER | A | 371 | −37.795 | 13.424 | −0.589 | 1.00 | 28.84 |
| ATOM | 2851 | N | SER | A | 372 | −38.571 | 14.340 | 1.312 | 1.00 | 27.84 |
| ATOM | 2852 | CA | SER | A | 372 | −38.300 | 15.689 | 0.845 | 1.00 | 27.18 |
| ATOM | 2853 | CB | SER | A | 372 | −38.896 | 16.737 | 1.789 | 1.00 | 27.36 |
| ATOM | 2854 | OG | SER | A | 372 | −38.331 | 16.609 | 3.080 | 1.00 | 28.50 |
| ATOM | 2855 | C | SER | A | 372 | −36.783 | 15.902 | 0.680 | 1.00 | 25.79 |
| ATOM | 2856 | O | SER | A | 372 | −36.358 | 16.690 | −0.173 | 1.00 | 26.29 |
| ATOM | 2857 | N | THR | A | 373 | −35.979 | 15.193 | 1.479 | 1.00 | 24.06 |
| ATOM | 2858 | CA | THR | A | 373 | −34.517 | 15.337 | 1.448 | 1.00 | 22.51 |
| ATOM | 2859 | CB | THR | A | 373 | −33.833 | 14.501 | 2.545 | 1.00 | 22.26 |
| ATOM | 2860 | OG1 | THR | A | 373 | −34.543 | 14.636 | 3.788 | 1.00 | 23.04 |
| ATOM | 2861 | CG2 | THR | A | 373 | −32.370 | 14.926 | 2.734 | 1.00 | 21.06 |
| ATOM | 2862 | C | THR | A | 373 | −33.984 | 14.906 | 0.076 | 1.00 | 21.61 |
| ATOM | 2863 | O | THR | A | 373 | −33.134 | 15.578 | −0.513 | 1.00 | 19.74 |
| ATOM | 2864 | N | PHE | A | 374 | −34.493 | 13.777 | −0.413 | 1.00 | 21.13 |
| ATOM | 2865 | CA | PHE | A | 374 | −34.137 | 13.280 | −1.749 | 1.00 | 22.10 |
| ATOM | 2866 | CB | PHE | A | 374 | −34.950 | 12.023 | −2.051 | 1.00 | 22.12 |
| ATOM | 2867 | CG | PHE | A | 374 | −34.624 | 11.380 | −3.366 | 1.00 | 22.90 |
| ATOM | 2868 | CD1 | PHE | A | 374 | −33.677 | 10.368 | −3.432 | 1.00 | 23.83 |
| ATOM | 2869 | CE1 | PHE | A | 374 | −33.381 | 9.749 | −4.649 | 1.00 | 22.56 |
| ATOM | 2870 | CZ | PHE | A | 374 | −34.041 | 10.162 | −5.802 | 1.00 | 23.24 |
| ATOM | 2871 | CE2 | PHE | A | 374 | −34.985 | 11.161 | −5.752 | 1.00 | 23.36 |
| ATOM | 2872 | CD2 | PHE | A | 374 | −35.280 | 11.769 | −4.523 | 1.00 | 23.73 |
| ATOM | 2873 | C | PHE | A | 374 | −34.343 | 14.349 | −2.818 | 1.00 | 22.24 |
| ATOM | 2874 | O | PHE | A | 374 | −33.413 | 14.681 | −3.548 | 1.00 | 22.63 |
| ATOM | 2875 | N | THR | A | 375 | −35.549 | 14.923 | −2.880 | 1.00 | 22.72 |
| ATOM | 2876 | CA | THR | A | 375 | −35.890 | 15.963 | −3.852 | 1.00 | 23.34 |
| ATOM | 2877 | CB | THR | A | 375 | −37.364 | 16.398 | −3.683 | 1.00 | 23.63 |
| ATOM | 2878 | OG1 | THR | A | 375 | −38.193 | 15.244 | −3.809 | 1.00 | 27.83 |
| ATOM | 2879 | CG2 | THR | A | 375 | −37.768 | 17.413 | −4.749 | 1.00 | 27.05 |
| ATOM | 2880 | C | THR | A | 375 | −35.003 | 17.203 | −3.746 | 1.00 | 22.66 |
| ATOM | 2881 | O | THR | A | 375 | −34.603 | 17.766 | −4.756 | 1.00 | 21.43 |
| ATOM | 2882 | N | ASN | A | 376 | −34.744 | 17.632 | −2.508 | 1.00 | 20.80 |
| ATOM | 2883 | CA | ASN | A | 376 | −33.880 | 18.766 | −2.207 | 1.00 | 21.33 |
| ATOM | 2884 | CB | ASN | A | 376 | −33.856 | 18.975 | −0.688 | 1.00 | 21.98 |
| ATOM | 2885 | CG | ASN | A | 376 | −33.343 | 20.354 | −0.278 | 1.00 | 27.01 |
| ATOM | 2886 | OD1 | ASN | A | 376 | −32.582 | 21.011 | −1.004 | 1.00 | 31.72 |
| ATOM | 2887 | ND2 | ASN | A | 376 | −33.748 | 20.793 | 0.913 | 1.00 | 30.14 |
| ATOM | 2888 | C | ASN | A | 376 | −32.465 | 18.527 | −2.733 | 1.00 | 19.80 |
| ATOM | 2889 | O | ASN | A | 376 | −31.898 | 19.389 | −3.415 | 1.00 | 19.75 |
| ATOM | 2890 | N | ILE | A | 377 | −31.915 | 17.354 | −2.431 | 1.00 | 19.00 |
| ATOM | 2891 | CA | ILE | A | 377 | −30.586 | 16.983 | −2.916 | 1.00 | 18.66 |
| ATOM | 2892 | CB | ILE | A | 377 | −30.081 | 15.651 | −2.319 | 1.00 | 18.56 |
| ATOM | 2893 | CG1 | ILE | A | 377 | −29.834 | 15.813 | −0.800 | 1.00 | 18.42 |
| ATOM | 2894 | CD1 | ILE | A | 377 | −29.634 | 14.481 | −0.028 | 1.00 | 19.35 |
| ATOM | 2895 | CG2 | ILE | A | 377 | −28.787 | 15.233 | −3.025 | 1.00 | 18.07 |
| ATOM | 2896 | C | ILE | A | 377 | −30.546 | 16.964 | −4.451 | 1.00 | 18.87 |
| ATOM | 2897 | O | ILE | A | 377 | −29.655 | 17.575 | −5.058 | 1.00 | 18.47 |
| ATOM | 2898 | N | ILE | A | 378 | −31.513 | 16.293 | −5.068 | 1.00 | 18.45 |
| ATOM | 2899 | CA | ILE | A | 378 | −31.556 | 16.216 | −6.539 | 1.00 | 19.78 |
| ATOM | 2900 | CB | ILE | A | 378 | −32.738 | 15.359 | −7.085 | 1.00 | 20.45 |
| ATOM | 2901 | CG1 | ILE | A | 378 | −32.593 | 13.891 | −6.650 | 1.00 | 22.32 |
| ATOM | 2902 | CD1 | ILE | A | 378 | −31.414 | 13.145 | −7.270 | 1.00 | 24.68 |
| ATOM | 2903 | CG2 | ILE | A | 378 | −32.829 | 15.472 | −8.646 | 1.00 | 21.27 |
| ATOM | 2904 | C | ILE | A | 378 | −31.561 | 17.588 | −7.177 | 1.00 | 19.82 |
| ATOM | 2905 | O | ILE | A | 378 | −30.760 | 17.849 | −8.101 | 1.00 | 19.62 |
| ATOM | 2906 | N | ASN | A | 379 | −32.441 | 18.470 | −6.689 | 1.00 | 18.68 |
| ATOM | 2907 | CA | ASN | A | 379 | −32.531 | 19.820 | −7.224 | 1.00 | 19.13 |
| ATOM | 2908 | CB | ASN | A | 379 | −33.738 | 20.578 | −6.658 | 1.00 | 20.08 |
| ATOM | 2909 | CG | ASN | A | 379 | −35.066 | 19.956 | −7.087 | 1.00 | 25.11 |
| ATOM | 2910 | OD1 | ASN | A | 379 | −35.121 | 19.173 | −8.044 | 1.00 | 29.37 |
| ATOM | 2911 | ND2 | ASN | A | 379 | −36.144 | 20.289 | −6.369 | 1.00 | 29.08 |
| ATOM | 2912 | C | ASN | A | 379 | −31.241 | 20.604 | −7.040 | 1.00 | 17.96 |
| ATOM | 2913 | O | ASN | A | 379 | −30.774 | 21.273 | −7.981 | 1.00 | 17.85 |
| ATOM | 2914 | N | ALA | A | 380 | −30.662 | 20.497 | −5.841 | 1.00 | 15.93 |
| ATOM | 2915 | CA | ALA | A | 380 | −29.458 | 21.241 | −5.509 | 1.00 | 16.08 |
| ATOM | 2916 | CB | ALA | A | 380 | −29.120 | 21.061 | −4.033 | 1.00 | 16.14 |
| ATOM | 2917 | C | ALA | A | 380 | −28.299 | 20.783 | −6.389 | 1.00 | 15.64 |
| ATOM | 2918 | O | ALA | A | 380 | −27.566 | 21.607 | −6.938 | 1.00 | 16.56 |
| ATOM | 2919 | N | VAL | A | 381 | −28.153 | 19.471 | −6.519 | 1.00 | 15.22 |
| ATOM | 2920 | CA | VAL | A | 381 | −27.039 | 18.912 | −7.302 | 1.00 | 15.77 |
| ATOM | 2921 | CB | VAL | A | 381 | −26.823 | 17.403 | −6.999 | 1.00 | 15.61 |
| ATOM | 2922 | CG1 | VAL | A | 381 | −25.747 | 16.777 | −7.940 | 1.00 | 14.83 |
| ATOM | 2923 | CG2 | VAL | A | 381 | −26.386 | 17.234 | −5.551 | 1.00 | 14.84 |
| ATOM | 2924 | C | VAL | A | 381 | −27.243 | 19.211 | −8.794 | 1.00 | 16.08 |
| ATOM | 2925 | O | VAL | A | 381 | −26.281 | 19.508 | −9.505 | 1.00 | 16.62 |
| ATOM | 2926 | N | SER | A | 382 | −28.482 | 19.112 | −9.278 | 1.00 | 16.66 |
| ATOM | 2927 | CA | SER | A | 382 | −28.772 | 19.453 | −10.690 | 1.00 | 18.67 |
| ATOM | 2928 | CB | SER | A | 382 | −30.246 | 19.212 | −11.043 | 1.00 | 18.26 |
| ATOM | 2929 | OG | SER | A | 382 | −30.538 | 17.855 | −10.893 | 1.00 | 24.56 |
| ATOM | 2930 | C | SER | A | 382 | −28.434 | 20.894 | −11.005 | 1.00 | 18.10 |
| ATOM | 2931 | O | SER | A | 382 | −27.815 | 21.183 | −12.027 | 1.00 | 18.05 |
| ATOM | 2932 | N | THR | A | 383 | −28.853 | 21.810 | −10.132 | 1.00 | 17.66 |
| ATOM | 2933 | CA | THR | A | 383 | −28.521 | 23.216 | −10.298 | 1.00 | 17.72 |
| ATOM | 2934 | CB | THR | A | 383 | −29.199 | 24.063 | −9.180 | 1.00 | 18.48 |
| ATOM | 2935 | OG1 | THR | A | 383 | −30.606 | 23.985 | −9.373 | 1.00 | 19.72 |
| ATOM | 2936 | CG2 | THR | A | 383 | −28.771 | 25.550 | −9.227 | 1.00 | 19.59 |
| ATOM | 2937 | C | THR | A | 383 | −27.017 | 23.470 | −10.314 | 1.00 | 17.09 |
| ATOM | 2938 | O | THR | A | 383 | −26.524 | 24.286 | −11.109 | 1.00 | 17.00 |
| ATOM | 2939 | N | TYR | A | 384 | −26.299 | 22.774 | −9.435 | 1.00 | 15.62 |
| ATOM | 2940 | CA | TYR | A | 384 | −24.858 | 22.925 | −9.312 | 1.00 | 15.37 |
| ATOM | 2941 | CB | TYR | A | 384 | −24.397 | 22.164 | −8.068 | 1.00 | 15.00 |
| ATOM | 2942 | CG | TYR | A | 384 | −22.958 | 22.345 | −7.630 | 1.00 | 15.08 |
| ATOM | 2943 | CD1 | TYR | A | 384 | −22.361 | 23.601 | −7.578 | 1.00 | 15.83 |
| ATOM | 2944 | CE1 | TYR | A | 384 | −21.049 | 23.752 | −7.131 | 1.00 | 16.33 |
| ATOM | 2945 | CZ | TYR | A | 384 | −20.321 | 22.623 | −6.737 | 1.00 | 16.13 |
| ATOM | 2946 | OH | TYR | A | 384 | −19.018 | 22.738 | −6.302 | 1.00 | 16.22 |
| ATOM | 2947 | CE2 | TYR | A | 384 | −20.890 | 21.386 | −6.778 | 1.00 | 13.72 |
| ATOM | 2948 | CD2 | TYR | A | 384 | −22.203 | 21.242 | −7.232 | 1.00 | 14.72 |
| ATOM | 2949 | C | TYR | A | 384 | −24.186 | 22.396 | −10.590 | 1.00 | 15.13 |
| ATOM | 2950 | O | TYR | A | 384 | −23.319 | 23.065 | −11.173 | 1.00 | 14.43 |
| ATOM | 2951 | N | ALA | A | 385 | −24.605 | 21.213 | −11.033 | 1.00 | 15.02 |
| ATOM | 2952 | CA | ALA | A | 385 | −24.045 | 20.639 | −12.263 | 1.00 | 15.60 |
| ATOM | 2953 | CB | ALA | A | 385 | −24.634 | 19.295 | −12.503 | 1.00 | 15.85 |
| ATOM | 2954 | C | ALA | A | 385 | −24.249 | 21.564 | −13.477 | 1.00 | 16.15 |
| ATOM | 2955 | O | ALA | A | 385 | −23.292 | 21.857 | −14.211 | 1.00 | 15.49 |
| ATOM | 2956 | N | ASP | A | 386 | −25.483 | 22.055 | −13.660 | 1.00 | 15.57 |
| ATOM | 2957 | CA | ASP | A | 386 | −25.782 | 23.058 | −14.694 | 1.00 | 16.09 |
| ATOM | 2958 | CB | ASP | A | 386 | −27.279 | 23.433 | −14.687 | 1.00 | 15.88 |
| ATOM | 2959 | CG | ASP | A | 386 | −28.158 | 22.379 | −15.349 | 1.00 | 18.85 |
| ATOM | 2960 | OD1 | ASP | A | 386 | −27.672 | 21.307 | −15.766 | 1.00 | 18.94 |
| ATOM | 2961 | OD2 | ASP | A | 386 | −29.365 | 22.624 | −15.461 | 1.00 | 23.88 |
| ATOM | 2962 | C | ASP | A | 386 | −24.938 | 24.322 | −14.526 | 1.00 | 15.81 |
| ATOM | 2963 | O | ASP | A | 386 | −24.594 | 24.998 | −15.501 | 1.00 | 16.29 |
| ATOM | 2964 | N | GLY | A | 387 | −24.591 | 24.640 | −13.290 | 1.00 | 15.76 |
| ATOM | 2965 | CA | GLY | A | 387 | −23.735 | 25.787 | −13.038 | 1.00 | 14.35 |
| ATOM | 2966 | C | GLY | A | 387 | −22.354 | 25.654 | −13.663 | 1.00 | 14.35 |
| ATOM | 2967 | O | GLY | A | 387 | −21.791 | 26.644 | −14.129 | 1.00 | 13.79 |
| ATOM | 2968 | N | PHE | A | 388 | −21.771 | 24.453 | −13.624 | 1.00 | 14.30 |
| ATOM | 2969 | CA | PHE | A | 388 | −20.479 | 24.217 | −14.312 | 1.00 | 14.69 |
| ATOM | 2970 | CB | PHE | A | 388 | −19.912 | 22.824 | −13.987 | 1.00 | 14.42 |
| ATOM | 2971 | CG | PHE | A | 388 | −19.359 | 22.730 | −12.584 | 1.00 | 13.79 |
| ATOM | 2972 | CD1 | PHE | A | 388 | −18.139 | 23.335 | −12.269 | 1.00 | 14.50 |
| ATOM | 2973 | CE1 | PHE | A | 388 | −17.621 | 23.261 | −10.947 | 1.00 | 16.38 |
| ATOM | 2974 | CZ | PHE | A | 388 | −18.377 | 22.627 | −9.951 | 1.00 | 14.77 |
| ATOM | 2975 | CE2 | PHE | A | 388 | −19.604 | 22.045 | −10.265 | 1.00 | 16.35 |
| ATOM | 2976 | CD2 | PHE | A | 388 | −20.088 | 22.100 | −11.578 | 1.00 | 14.00 |
| ATOM | 2977 | C | PHE | A | 388 | −20.601 | 24.428 | −15.821 | 1.00 | 15.31 |
| ATOM | 2978 | O | PHE | A | 388 | −19.740 | 25.078 | −16.440 | 1.00 | 15.55 |
| ATOM | 2979 | N | LEU | A | 389 | −21.669 | 23.913 | −16.415 | 1.00 | 16.52 |
| ATOM | 2980 | CA | LEU | A | 389 | −21.889 | 24.159 | −17.856 | 1.00 | 17.84 |
| ATOM | 2981 | CB | LEU | A | 389 | −23.137 | 23.431 | −18.382 | 1.00 | 17.83 |
| ATOM | 2982 | CG | LEU | A | 389 | −23.172 | 21.911 | −18.427 | 1.00 | 22.75 |
| ATOM | 2983 | CD1 | LEU | A | 389 | −24.247 | 21.418 | −19.401 | 1.00 | 22.55 |
| ATOM | 2984 | CD2 | LEU | A | 389 | −21.805 | 21.333 | −18.806 | 1.00 | 24.63 |
| ATOM | 2985 | C | LEU | A | 389 | −22.013 | 25.634 | −18.136 | 1.00 | 18.32 |
| ATOM | 2986 | O | LEU | A | 389 | −21.409 | 26.138 | −19.091 | 1.00 | 19.50 |
| ATOM | 2987 | N | SER | A | 390 | −22.775 | 26.341 | −17.295 | 1.00 | 18.55 |
| ATOM | 2988 | CA | SER | A | 390 | −23.021 | 27.767 | −17.469 | 1.00 | 19.33 |
| ATOM | 2989 | CB | SER | A | 390 | −24.090 | 28.246 | −16.491 | 1.00 | 19.89 |
| ATOM | 2990 | OG | SER | A | 390 | −25.325 | 27.693 | −16.891 | 1.00 | 24.07 |
| ATOM | 2991 | C | SER | A | 390 | −21.763 | 28.603 | −17.323 | 1.00 | 19.51 |
| ATOM | 2992 | O | SER | A | 390 | −21.575 | 29.585 | −18.055 | 1.00 | 19.69 |
| ATOM | 2993 | N | GLU | A | 391 | −20.893 | 28.200 | −16.399 | 1.00 | 18.87 |
| ATOM | 2994 | CA | GLU | A | 391 | −19.633 | 28.879 | −16.220 | 1.00 | 19.59 |
| ATOM | 2995 | CB | GLU | A | 391 | −18.901 | 28.393 | −14.952 | 1.00 | 20.02 |
| ATOM | 2996 | CG | GLU | A | 391 | −19.528 | 28.924 | −13.668 | 1.00 | 23.54 |
| ATOM | 2997 | CD | GLU | A | 391 | −19.590 | 30.448 | −13.634 | 1.00 | 26.61 |
| ATOM | 2998 | OE1 | GLU | A | 391 | −18.609 | 31.102 | −14.023 | 1.00 | 28.42 |
| ATOM | 2999 | OE2 | GLU | A | 391 | −20.637 | 30.994 | −13.227 | 1.00 | 29.52 |
| ATOM | 3000 | C | GLU | A | 391 | −18.738 | 28.729 | −17.457 | 1.00 | 19.12 |
| ATOM | 3001 | O | GLU | A | 391 | −18.123 | 29.709 | −17.906 | 1.00 | 19.40 |
| ATOM | 3002 | N | ALA | A | 392 | −18.654 | 27.516 | −17.991 | 1.00 | 18.81 |
| ATOM | 3003 | CA | ALA | A | 392 | −17.861 | 27.304 | −19.201 | 1.00 | 19.72 |
| ATOM | 3004 | CB | ALA | A | 392 | −17.758 | 25.815 | −19.526 | 1.00 | 19.45 |
| ATOM | 3005 | C | ALA | A | 392 | −18.478 | 28.098 | −20.363 | 1.00 | 19.59 |
| ATOM | 3006 | O | ALA | A | 392 | −17.764 | 28.724 | −21.157 | 1.00 | 18.65 |
| ATOM | 3007 | N | ALA | A | 393 | −19.808 | 28.117 | −20.425 | 1.00 | 20.40 |
| ATOM | 3008 | CA | ALA | A | 393 | −20.526 | 28.820 | −21.507 | 1.00 | 21.27 |
| ATOM | 3009 | CB | ALA | A | 393 | −22.035 | 28.524 | −21.421 | 1.00 | 21.83 |
| ATOM | 3010 | C | ALA | A | 393 | −20.266 | 30.334 | −21.574 | 1.00 | 21.79 |
| ATOM | 3011 | O | ALA | A | 393 | −20.283 | 30.920 | −22.664 | 1.00 | 21.96 |
| ATOM | 3012 | N | LYS | A | 394 | −19.976 | 30.971 | −20.435 | 1.00 | 21.37 |
| ATOM | 3013 | CA | LYS | A | 394 | −19.626 | 32.383 | −20.447 | 1.00 | 22.02 |
| ATOM | 3014 | CB | LYS | A | 394 | −19.289 | 32.916 | −19.043 | 1.00 | 23.11 |
| ATOM | 3015 | CG | LYS | A | 394 | −20.411 | 32.980 | −18.044 | 1.00 | 25.98 |
| ATOM | 3016 | CD | LYS | A | 394 | −19.782 | 33.341 | −16.700 | 1.00 | 28.89 |
| ATOM | 3017 | CE | LYS | A | 394 | −20.793 | 33.314 | −15.576 | 1.00 | 34.05 |
| ATOM | 3018 | NZ | LYS | A | 394 | −20.097 | 33.674 | −14.290 | 1.00 | 33.96 |
| ATOM | 3019 | C | LYS | A | 394 | −18.403 | 32.626 | −21.310 | 1.00 | 21.55 |
| ATOM | 3020 | O | LYS | A | 394 | −18.188 | 33.742 | −21.771 | 1.00 | 22.41 |
| ATOM | 3021 | N | TYR | A | 395 | −17.570 | 31.604 | −21.488 | 1.00 | 19.98 |
| ATOM | 3022 | CA | TYR | A | 395 | −16.287 | 31.824 | −22.132 | 1.00 | 19.46 |
| ATOM | 3023 | CB | TYR | A | 395 | −15.137 | 31.464 | −21.185 | 1.00 | 20.82 |
| ATOM | 3024 | CG | TYR | A | 395 | −15.291 | 32.165 | −19.872 | 1.00 | 22.04 |
| ATOM | 3025 | CD1 | TYR | A | 395 | −15.644 | 31.450 | −18.716 | 1.00 | 23.21 |
| ATOM | 3026 | CE1 | TYR | A | 395 | −15.806 | 32.097 | −17.508 | 1.00 | 23.70 |
| ATOM | 3027 | CZ | TYR | A | 395 | −15.661 | 33.473 | −17.460 | 1.00 | 23.93 |
| ATOM | 3028 | OH | TYR | A | 395 | −15.828 | 34.143 | −16.272 | 1.00 | 26.14 |
| ATOM | 3029 | CE2 | TYR | A | 395 | −15.327 | 34.202 | −18.593 | 1.00 | 24.38 |
| ATOM | 3030 | CD2 | TYR | A | 395 | −15.157 | 33.548 | −19.791 | 1.00 | 22.37 |
| ATOM | 3031 | C | TYR | A | 395 | −16.157 | 31.119 | −23.451 | 1.00 | 19.22 |
| ATOM | 3032 | O | TYR | A | 395 | −15.045 | 30.940 | −23.941 | 1.00 | 18.70 |
| ATOM | 3033 | N | VAL | A | 396 | −17.299 | 30.718 | −24.018 | 1.00 | 18.16 |
| ATOM | 3034 | CA | VAL | A | 396 | −17.331 | 30.135 | −25.352 | 1.00 | 19.03 |
| ATOM | 3035 | CB | VAL | A | 396 | −18.396 | 29.025 | −25.458 | 1.00 | 18.18 |
| ATOM | 3036 | CG1 | VAL | A | 396 | −18.469 | 28.465 | −26.898 | 1.00 | 18.63 |
| ATOM | 3037 | CG2 | VAL | A | 396 | −18.094 | 27.915 | −24.452 | 1.00 | 18.97 |
| ATOM | 3038 | C | VAL | A | 396 | −17.654 | 31.288 | −26.308 | 1.00 | 19.65 |
| ATOM | 3039 | O | VAL | A | 396 | −18.644 | 31.986 | −26.098 | 1.00 | 19.66 |
| ATOM | 3040 | N | PRO | A | 397 | −16.810 | 31.507 | −27.328 | 1.00 | 20.41 |
| ATOM | 3041 | CA | PRO | A | 397 | −17.016 | 32.626 | −28.256 | 1.00 | 20.82 |
| ATOM | 3042 | CB | PRO | A | 397 | −15.794 | 32.561 | −29.175 | 1.00 | 21.47 |
| ATOM | 3043 | CG | PRO | A | 397 | −14.819 | 31.725 | −28.475 | 1.00 | 21.69 |
| ATOM | 3044 | CD | PRO | A | 397 | −15.598 | 30.741 | −27.661 | 1.00 | 19.70 |
| ATOM | 3045 | C | PRO | A | 397 | −18.280 | 32.434 | −29.073 | 1.00 | 21.11 |
| ATOM | 3046 | O | PRO | A | 397 | −18.844 | 31.339 | −29.088 | 1.00 | 19.88 |
| ATOM | 3047 | N | ALA | A | 398 | −18.713 | 33.492 | −29.765 | 1.00 | 21.26 |
| ATOM | 3048 | CA | ALA | A | 398 | −19.951 | 33.424 | −30.559 | 1.00 | 21.44 |
| ATOM | 3049 | CB | ALA | A | 398 | −20.227 | 34.766 | −31.230 | 1.00 | 22.38 |
| ATOM | 3050 | C | ALA | A | 398 | −19.971 | 32.297 | −31.587 | 1.00 | 21.30 |
| ATOM | 3051 | O | ALA | A | 398 | −21.038 | 31.769 | −31.901 | 1.00 | 22.15 |
| ATOM | 3052 | N | ASP | A | 399 | −18.804 | 31.896 | −32.102 | 1.00 | 20.30 |
| ATOM | 3053 | CA | ASP | A | 399 | −18.780 | 30.858 | −33.133 | 1.00 | 19.40 |
| ATOM | 3054 | CB | ASP | A | 399 | −17.587 | 31.032 | −34.071 | 1.00 | 19.42 |
| ATOM | 3055 | CG | ASP | A | 399 | −16.233 | 30.835 | −33.381 | 1.00 | 21.84 |
| ATOM | 3056 | OD1 | ASP | A | 399 | −16.146 | 30.569 | −32.159 | 1.00 | 20.91 |
| ATOM | 3057 | OD2 | ASP | A | 399 | −15.229 | 30.950 | −34.104 | 1.00 | 24.62 |
| ATOM | 3058 | C | ASP | A | 399 | −18.834 | 29.435 | −32.579 | 1.00 | 18.14 |
| ATOM | 3059 | O | ASP | A | 399 | −18.802 | 28.465 | −33.350 | 1.00 | 16.78 |
| ATOM | 3060 | N | GLY | A | 400 | −18.891 | 29.322 | −31.245 | 1.00 | 16.82 |
| ATOM | 3061 | CA | GLY | A | 400 | −18.996 | 28.015 | −30.607 | 1.00 | 15.41 |
| ATOM | 3062 | C | GLY | A | 400 | −17.693 | 27.229 | −30.556 | 1.00 | 15.07 |
| ATOM | 3063 | O | GLY | A | 400 | −17.704 | 26.041 | −30.203 | 1.00 | 15.23 |
| ATOM | 3064 | N | SER | A | 401 | −16.572 | 27.861 | −30.882 | 1.00 | 14.21 |
| ATOM | 3065 | CA | SER | A | 401 | −15.312 | 27.119 | −30.893 | 1.00 | 14.76 |
| ATOM | 3066 | CB | SER | A | 401 | −14.241 | 27.840 | −31.718 | 1.00 | 14.71 |
| ATOM | 3067 | OG | SER | A | 401 | −14.059 | 29.160 | −31.257 | 1.00 | 16.86 |
| ATOM | 3068 | C | SER | A | 401 | −14.815 | 26.866 | −29.448 | 1.00 | 14.38 |
| ATOM | 3069 | O | SER | A | 401 | −14.992 | 27.717 | −28.562 | 1.00 | 14.58 |
| ATOM | 3070 | N | LEU | A | 402 | −14.169 | 25.720 | −29.249 | 1.00 | 13.54 |
| ATOM | 3071 | CA | LEU | A | 402 | −13.603 | 25.364 | −27.968 | 1.00 | 13.21 |
| ATOM | 3072 | CB | LEU | A | 402 | −14.271 | 24.080 | −27.450 | 1.00 | 13.42 |
| ATOM | 3073 | CG | LEU | A | 402 | −15.776 | 24.192 | −27.162 | 1.00 | 14.06 |
| ATOM | 3074 | CD1 | LEU | A | 402 | −16.289 | 22.834 | −26.668 | 1.00 | 13.87 |
| ATOM | 3075 | CD2 | LEU | A | 402 | −15.997 | 25.264 | −26.109 | 1.00 | 17.00 |
| ATOM | 3076 | C | LEU | A | 402 | −12.111 | 25.143 | −28.109 | 1.00 | 12.99 |
| ATOM | 3077 | O | LEU | A | 402 | −11.695 | 24.166 | −28.707 | 1.00 | 13.19 |
| ATOM | 3078 | N | ALA | A | 403 | −11.320 | 26.070 | −27.578 | 1.00 | 13.02 |
| ATOM | 3079 | CA | ALA | A | 403 | −9.884 | 25.870 | −27.454 | 1.00 | 12.06 |
| ATOM | 3080 | CB | ALA | A | 403 | −9.194 | 27.226 | −27.220 | 1.00 | 11.77 |
| ATOM | 3081 | C | ALA | A | 403 | −9.591 | 24.907 | −26.300 | 1.00 | 12.81 |
| ATOM | 3082 | O | ALA | A | 403 | −10.508 | 24.315 | −25.714 | 1.00 | 12.36 |
| ATOM | 3083 | N | GLU | A | 404 | −8.308 | 24.772 | −25.959 | 1.00 | 11.39 |
| ATOM | 3084 | CA | GLU | A | 404 | −7.918 | 23.922 | −24.855 | 1.00 | 11.47 |
| ATOM | 3085 | CB | GLU | A | 404 | −6.412 | 23.689 | −24.931 | 1.00 | 11.02 |
| ATOM | 3086 | CG | GLU | A | 404 | −5.865 | 22.723 | −23.873 | 1.00 | 11.30 |
| ATOM | 3087 | CD | GLU | A | 404 | −4.363 | 22.669 | −23.954 | 1.00 | 12.25 |
| ATOM | 3088 | OE1 | GLU | A | 404 | −3.729 | 23.692 | −23.622 | 1.00 | 12.06 |
| ATOM | 3089 | OE2 | GLU | A | 404 | −3.818 | 21.624 | −24.390 | 1.00 | 12.60 |
| ATOM | 3090 | C | GLU | A | 404 | −8.246 | 24.635 | −23.538 | 1.00 | 11.77 |
| ATOM | 3091 | O | GLU | A | 404 | −8.755 | 24.006 | −22.590 | 1.00 | 11.08 |
| ATOM | 3092 | N | GLN | A | 405 | −7.890 | 25.924 | −23.453 | 1.00 | 12.07 |
| ATOM | 3093 | CA | GLN | A | 405 | −7.952 | 26.655 | −22.196 | 1.00 | 13.33 |
| ATOM | 3094 | CB | GLN | A | 405 | −6.539 | 26.986 | −21.678 | 1.00 | 13.24 |
| ATOM | 3095 | CG | GLN | A | 405 | −5.625 | 25.821 | −21.553 | 1.00 | 15.96 |
| ATOM | 3096 | CD | GLN | A | 405 | −4.229 | 26.213 | −21.051 | 1.00 | 14.97 |
| ATOM | 3097 | OE1 | GLN | A | 405 | −4.068 | 27.130 | −20.236 | 1.00 | 15.09 |
| ATOM | 3098 | NE2 | GLN | A | 405 | −3.236 | 25.475 | −21.496 | 1.00 | 15.87 |
| ATOM | 3099 | C | GLN | A | 405 | −8.687 | 27.985 | −22.356 | 1.00 | 13.51 |
| ATOM | 3100 | O | GLN | A | 405 | −8.870 | 28.475 | −23.489 | 1.00 | 13.74 |
| ATOM | 3101 | N | PHE | A | 406 | −9.041 | 28.587 | −21.226 | 1.00 | 12.60 |
| ATOM | 3102 | CA | PHE | A | 406 | −9.516 | 29.990 | −21.207 | 1.00 | 13.82 |
| ATOM | 3103 | CB | PHE | A | 406 | −11.058 | 30.104 | −21.232 | 1.00 | 13.56 |
| ATOM | 3104 | CG | PHE | A | 406 | −11.800 | 29.385 | −20.123 | 1.00 | 15.20 |
| ATOM | 3105 | CD1 | PHE | A | 406 | −12.155 | 28.026 | −20.242 | 1.00 | 16.33 |
| ATOM | 3106 | CE1 | PHE | A | 406 | −12.879 | 27.370 | −19.239 | 1.00 | 16.80 |
| ATOM | 3107 | CZ | PHE | A | 406 | −13.340 | 28.094 | −18.114 | 1.00 | 16.74 |
| ATOM | 3108 | CE2 | PHE | A | 406 | −13.020 | 29.453 | −17.993 | 1.00 | 15.37 |
| ATOM | 3109 | CD2 | PHE | A | 406 | −12.260 | 30.101 | −19.000 | 1.00 | 17.56 |
| ATOM | 3110 | C | PHE | A | 406 | −8.836 | 30.737 | −20.078 | 1.00 | 13.80 |
| ATOM | 3111 | O | PHE | A | 406 | −8.587 | 30.151 | −19.018 | 1.00 | 14.02 |
| ATOM | 3112 | N | ASP | A | 407 | −8.481 | 31.997 | −20.321 | 1.00 | 14.98 |
| ATOM | 3113 | CA | ASP | A | 407 | −7.547 | 32.726 | −19.438 | 1.00 | 15.04 |
| ATOM | 3114 | CB | ASP | A | 407 | −7.237 | 34.115 | −20.032 | 1.00 | 15.99 |
| ATOM | 3115 | CG | ASP | A | 407 | −6.159 | 34.829 | −19.293 | 1.00 | 18.17 |
| ATOM | 3116 | OD1 | ASP | A | 407 | −6.474 | 35.508 | −18.293 | 1.00 | 20.41 |
| ATOM | 3117 | OD2 | ASP | A | 407 | −4.993 | 34.683 | −19.685 | 1.00 | 20.75 |
| ATOM | 3118 | C | ASP | A | 407 | −8.100 | 32.829 | −18.005 | 1.00 | 14.54 |
| ATOM | 3119 | O | ASP | A | 407 | −9.257 | 33.185 | −17.800 | 1.00 | 15.12 |
| ATOM | 3120 | N | ARG | A | 408 | −7.248 | 32.548 | −17.018 | 1.00 | 14.63 |
| ATOM | 3121 | CA | ARG | A | 408 | −7.644 | 32.530 | −15.609 | 1.00 | 15.33 |
| ATOM | 3122 | CB | ARG | A | 408 | −6.453 | 32.095 | −14.753 | 1.00 | 15.32 |
| ATOM | 3123 | CG | ARG | A | 408 | −5.236 | 33.062 | −14.828 | 1.00 | 14.48 |
| ATOM | 3124 | CD | ARG | A | 408 | −4.009 | 32.479 | −14.122 | 1.00 | 16.22 |
| ATOM | 3125 | NE | ARG | A | 408 | −4.237 | 32.248 | −12.695 | 1.00 | 15.55 |
| ATOM | 3126 | CZ | ARG | A | 408 | −3.613 | 31.323 | −11.961 | 1.00 | 18.66 |
| ATOM | 3127 | NH1 | ARG | A | 408 | −3.878 | 31.231 | −10.658 | 1.00 | 17.51 |
| ATOM | 3128 | NH2 | ARG | A | 408 | −2.717 | 30.499 | −12.511 | 1.00 | 17.12 |
| ATOM | 3129 | C | ARG | A | 408 | −8.167 | 33.886 | −15.108 | 1.00 | 16.66 |
| ATOM | 3130 | O | ARG | A | 408 | −8.898 | 33.943 | −14.110 | 1.00 | 16.82 |
| ATOM | 3131 | N | ASN | A | 409 | −7.781 | 34.964 | −15.790 | 1.00 | 18.00 |
| ATOM | 3132 | CA | ASN | A | 409 | −8.252 | 36.316 | −15.421 | 1.00 | 20.06 |
| ATOM | 3133 | CB | ASN | A | 409 | −7.069 | 37.275 | −15.355 | 1.00 | 20.32 |
| ATOM | 3134 | CG | ASN | A | 409 | −6.119 | 36.937 | −14.224 | 1.00 | 21.65 |
| ATOM | 3135 | OD1 | ASN | A | 409 | −6.549 | 36.678 | −13.111 | 1.00 | 24.42 |
| ATOM | 3136 | ND2 | ASN | A | 409 | −4.830 | 36.914 | −14.516 | 1.00 | 23.96 |
| ATOM | 3137 | C | ASN | A | 409 | −9.320 | 36.903 | −16.336 | 1.00 | 21.43 |
| ATOM | 3138 | O | ASN | A | 409 | −10.272 | 37.524 | −15.857 | 1.00 | 22.03 |
| ATOM | 3139 | N | SER | A | 410 | −9.152 | 36.724 | −17.646 | 1.00 | 22.25 |
| ATOM | 3140 | CA | SER | A | 410 | −10.007 | 37.410 | −18.624 | 1.00 | 22.76 |
| ATOM | 3141 | CB | SER | A | 410 | −9.146 | 38.159 | −19.636 | 1.00 | 23.45 |
| ATOM | 3142 | OG | SER | A | 410 | −8.470 | 37.260 | −20.495 | 1.00 | 23.38 |
| ATOM | 3143 | C | SER | A | 410 | −10.971 | 36.472 | −19.343 | 1.00 | 22.82 |
| ATOM | 3144 | O | SER | A | 410 | −11.898 | 36.925 | −20.010 | 1.00 | 23.59 |
| ATOM | 3145 | N | GLY | A | 411 | −10.758 | 35.161 | −19.238 | 1.00 | 21.64 |
| ATOM | 3146 | CA | GLY | A | 411 | −11.668 | 34.221 | −19.877 | 1.00 | 20.39 |
| ATOM | 3147 | C | GLY | A | 411 | −11.476 | 34.087 | −21.379 | 1.00 | 19.99 |
| ATOM | 3148 | O | GLY | A | 411 | −12.223 | 33.368 | −22.029 | 1.00 | 20.85 |
| ATOM | 3149 | N | THR | A | 412 | −10.478 | 34.750 | −21.941 | 1.00 | 19.04 |
| ATOM | 3150 | CA | THR | A | 412 | −10.268 | 34.658 | −23.383 | 1.00 | 20.25 |
| ATOM | 3151 | CB | THR | A | 412 | −9.447 | 35.853 | −23.922 | 1.00 | 21.81 |
| ATOM | 3152 | OG1 | THR | A | 412 | −8.187 | 35.900 | −23.257 | 1.00 | 26.24 |
| ATOM | 3153 | CG2 | THR | A | 412 | −10.160 | 37.163 | −23.631 | 1.00 | 23.82 |
| ATOM | 3154 | C | THR | A | 412 | −9.615 | 33.294 | −23.732 | 1.00 | 19.17 |
| ATOM | 3155 | O | THR | A | 412 | −8.786 | 32.796 | −22.970 | 1.00 | 17.70 |
| ATOM | 3156 | N | PRO | A | 413 | −9.996 | 32.688 | −24.874 | 1.00 | 18.84 |
| ATOM | 3157 | CA | PRO | A | 413 | −9.466 | 31.348 | −25.234 | 1.00 | 18.32 |
| ATOM | 3158 | CB | PRO | A | 413 | −10.220 | 31.002 | −26.525 | 1.00 | 18.75 |
| ATOM | 3159 | CG | PRO | A | 413 | −11.513 | 31.928 | −26.451 | 1.00 | 19.42 |
| ATOM | 3160 | CD | PRO | A | 413 | −10.943 | 33.195 | −25.891 | 1.00 | 19.40 |
| ATOM | 3161 | C | PRO | A | 413 | −7.959 | 31.399 | −25.464 | 1.00 | 18.87 |
| ATOM | 3162 | O | PRO | A | 413 | −7.436 | 32.406 | −25.955 | 1.00 | 18.20 |
| ATOM | 3163 | N | LEU | A | 414 | −7.253 | 30.353 | −25.051 | 1.00 | 18.44 |
| ATOM | 3164 | CA | LEU | A | 414 | −5.822 | 30.275 | −25.305 | 1.00 | 18.90 |
| ATOM | 3165 | CB | LEU | A | 414 | −4.992 | 30.974 | −24.208 | 1.00 | 21.66 |
| ATOM | 3166 | CG | LEU | A | 414 | −5.019 | 30.574 | −22.754 | 1.00 | 24.35 |
| ATOM | 3167 | CD1 | LEU | A | 414 | −4.134 | 31.484 | −21.892 | 1.00 | 27.42 |
| ATOM | 3168 | CD2 | LEU | A | 414 | −6.406 | 30.669 | −22.224 | 1.00 | 30.77 |
| ATOM | 3169 | C | LEU | A | 414 | −5.362 | 28.854 | −25.518 | 1.00 | 17.24 |
| ATOM | 3170 | O | LEU | A | 414 | −6.138 | 27.913 | −25.406 | 1.00 | 15.97 |
| ATOM | 3171 | N | SER | A | 415 | −4.091 | 28.733 | −25.865 | 1.00 | 15.34 |
| ATOM | 3172 | CA | SER | A | 415 | −3.473 | 27.481 | −26.257 | 1.00 | 15.28 |
| ATOM | 3173 | CB | SER | A | 415 | −3.434 | 26.468 | −25.101 | 1.00 | 14.94 |
| ATOM | 3174 | OG | SER | A | 415 | −2.632 | 25.355 | −25.445 | 1.00 | 14.00 |
| ATOM | 3175 | C | SER | A | 415 | −4.141 | 26.932 | −27.528 | 1.00 | 15.13 |
| ATOM | 3176 | O | SER | A | 415 | −4.665 | 27.718 | −28.334 | 1.00 | 14.92 |
| ATOM | 3177 | N | ALA | A | 416 | −4.097 | 25.618 | −27.714 | 1.00 | 14.27 |
| ATOM | 3178 | CA | ALA | A | 416 | −4.540 | 24.977 | −28.976 | 1.00 | 14.25 |
| ATOM | 3179 | CB | ALA | A | 416 | −4.380 | 23.486 | −28.889 | 1.00 | 13.68 |
| ATOM | 3180 | C | ALA | A | 416 | −5.981 | 25.314 | −29.315 | 1.00 | 14.33 |
| ATOM | 3181 | O | ALA | A | 416 | −6.854 | 25.216 | −28.459 | 1.00 | 14.09 |
| ATOM | 3182 | N | LEU | A | 417 | −6.223 | 25.680 | −30.567 | 1.00 | 13.34 |
| ATOM | 3183 | CA | LEU | A | 417 | −7.584 | 25.985 | −31.006 | 1.00 | 14.22 |
| ATOM | 3184 | CB | LEU | A | 417 | −7.536 | 26.931 | −32.194 | 1.00 | 16.32 |
| ATOM | 3185 | CG | LEU | A | 417 | −6.841 | 28.283 | −31.942 | 1.00 | 18.75 |
| ATOM | 3186 | CD1 | LEU | A | 417 | −7.005 | 29.127 | −33.163 | 1.00 | 23.81 |
| ATOM | 3187 | CD2 | LEU | A | 417 | −7.419 | 28.991 | −30.712 | 1.00 | 21.76 |
| ATOM | 3188 | C | LEU | A | 417 | −8.279 | 24.687 | −31.413 | 1.00 | 13.18 |
| ATOM | 3189 | O | LEU | A | 417 | −7.610 | 23.712 | −31.775 | 1.00 | 13.19 |
| ATOM | 3190 | N | HIS | A | 418 | −9.609 | 24.658 | −31.311 | 1.00 | 12.26 |
| ATOM | 3191 | CA | HIS | A | 418 | −10.399 | 23.496 | −31.764 | 1.00 | 11.77 |
| ATOM | 3192 | CB | HIS | A | 418 | −10.487 | 23.454 | −33.303 | 1.00 | 13.41 |
| ATOM | 3193 | CG | HIS | A | 418 | −11.294 | 24.566 | −33.898 | 1.00 | 14.68 |
| ATOM | 3194 | ND1 | HIS | A | 418 | −12.646 | 24.717 | −33.660 | 1.00 | 16.00 |
| ATOM | 3195 | CE1 | HIS | A | 418 | −13.095 | 25.762 | −34.341 | 1.00 | 17.83 |
| ATOM | 3196 | NE2 | HIS | A | 418 | −12.085 | 26.290 | −35.015 | 1.00 | 17.38 |
| ATOM | 3197 | CD2 | HIS | A | 418 | −10.948 | 25.557 | −34.763 | 1.00 | 17.65 |
| ATOM | 3198 | C | HIS | A | 418 | −9.826 | 22.187 | −31.206 | 1.00 | 12.24 |
| ATOM | 3199 | O | HIS | A | 418 | −9.540 | 21.250 | −31.947 | 1.00 | 12.10 |
| ATOM | 3200 | N | LEU | A | 419 | −9.656 | 22.116 | −29.880 | 1.00 | 10.99 |
| ATOM | 3201 | CA | LEU | A | 419 | −9.152 | 20.881 | −29.301 | 1.00 | 10.46 |
| ATOM | 3202 | CB | LEU | A | 419 | −8.742 | 21.069 | −27.826 | 1.00 | 10.91 |
| ATOM | 3203 | CG | LEU | A | 419 | −7.983 | 19.883 | −27.220 | 1.00 | 10.16 |
| ATOM | 3204 | CD1 | LEU | A | 419 | −6.524 | 19.944 | −27.669 | 1.00 | 11.45 |
| ATOM | 3205 | CD2 | LEU | A | 419 | −8.080 | 19.960 | −25.657 | 1.00 | 10.73 |
| ATOM | 3206 | C | LEU | A | 419 | −10.215 | 19.812 | −29.398 | 1.00 | 10.49 |
| ATOM | 3207 | O | LEU | A | 419 | −11.312 | 19.973 | −28.863 | 1.00 | 10.67 |
| ATOM | 3208 | N | THR | A | 420 | −9.860 | 18.686 | −30.021 | 1.00 | 10.22 |
| ATOM | 3209 | CA | THR | A | 420 | −10.833 | 17.629 | −30.296 | 1.00 | 10.90 |
| ATOM | 3210 | CB | THR | A | 420 | −10.201 | 16.460 | −31.096 | 1.00 | 11.55 |
| ATOM | 3211 | OG1 | THR | A | 420 | −9.357 | 16.999 | −32.115 | 1.00 | 12.00 |
| ATOM | 3212 | CG2 | THR | A | 420 | −11.310 | 15.625 | −31.786 | 1.00 | 12.52 |
| ATOM | 3213 | C | THR | A | 420 | −11.426 | 17.135 | −28.995 | 1.00 | 11.43 |
| ATOM | 3214 | O | THR | A | 420 | −12.648 | 16.903 | −28.891 | 1.00 | 11.83 |
| ATOM | 3215 | N | TRP | A | 421 | −10.569 | 16.987 | −27.980 | 1.00 | 9.79 |
| ATOM | 3216 | CA | TRP | A | 421 | −11.052 | 16.511 | −26.688 | 1.00 | 11.67 |
| ATOM | 3217 | CB | TRP | A | 421 | −9.839 | 16.184 | −25.803 | 1.00 | 12.06 |
| ATOM | 3218 | CG | TRP | A | 421 | −10.075 | 15.476 | −24.508 | 1.00 | 11.94 |
| ATOM | 3219 | CD1 | TRP | A | 421 | −11.274 | 15.202 | −23.881 | 1.00 | 14.46 |
| ATOM | 3220 | NE1 | TRP | A | 421 | −11.044 | 14.590 | −22.663 | 1.00 | 14.38 |
| ATOM | 3221 | CE2 | TRP | A | 421 | −9.691 | 14.497 | −22.468 | 1.00 | 13.52 |
| ATOM | 3222 | CD2 | TRP | A | 421 | −9.053 | 15.039 | −23.616 | 1.00 | 13.01 |
| ATOM | 3223 | CE3 | TRP | A | 421 | −7.652 | 15.045 | −23.680 | 1.00 | 15.38 |
| ATOM | 3224 | CZ3 | TRP | A | 421 | −6.932 | 14.503 | −22.605 | 1.00 | 16.10 |
| ATOM | 3225 | CH2 | TRP | A | 421 | −7.603 | 13.973 | −21.475 | 1.00 | 14.87 |
| ATOM | 3226 | CZ2 | TRP | A | 421 | −8.973 | 13.945 | −21.398 | 1.00 | 14.27 |
| ATOM | 3227 | C | TRP | A | 421 | −12.035 | 17.514 | −26.032 | 1.00 | 10.89 |
| ATOM | 3228 | O | TRP | A | 421 | −12.966 | 17.092 | −25.357 | 1.00 | 10.96 |
| ATOM | 3229 | N | SER | A | 422 | −11.844 | 18.822 | −26.211 | 1.00 | 10.49 |
| ATOM | 3230 | CA | SER | A | 422 | −12.833 | 19.794 | −25.696 | 1.00 | 11.13 |
| ATOM | 3231 | CB | SER | A | 422 | −12.459 | 21.243 | −26.049 | 1.00 | 10.97 |
| ATOM | 3232 | OG | SER | A | 422 | −11.302 | 21.682 | −25.338 | 1.00 | 13.73 |
| ATOM | 3233 | C | SER | A | 422 | −14.229 | 19.496 | −26.257 | 1.00 | 11.40 |
| ATOM | 3234 | O | SER | A | 422 | −15.204 | 19.468 | −25.530 | 1.00 | 12.11 |
| ATOM | 3235 | N | TYR | A | 423 | −14.320 | 19.281 | −27.563 | 1.00 | 11.29 |
| ATOM | 3236 | CA | TYR | A | 423 | −15.617 | 18.990 | −28.170 | 1.00 | 11.29 |
| ATOM | 3237 | CB | TYR | A | 423 | −15.502 | 19.020 | −29.717 | 1.00 | 11.85 |
| ATOM | 3238 | CG | TYR | A | 423 | −15.132 | 20.389 | −30.274 | 1.00 | 12.18 |
| ATOM | 3239 | CD1 | TYR | A | 423 | −16.002 | 21.485 | −30.145 | 1.00 | 10.35 |
| ATOM | 3240 | CE1 | TYR | A | 423 | −15.668 | 22.741 | −30.643 | 1.00 | 11.83 |
| ATOM | 3241 | CZ | TYR | A | 423 | −14.468 | 22.912 | −31.316 | 1.00 | 12.29 |
| ATOM | 3242 | OH | TYR | A | 423 | −14.157 | 24.145 | −31.783 | 1.00 | 13.08 |
| ATOM | 3243 | CE2 | TYR | A | 423 | −13.588 | 21.845 | −31.496 | 1.00 | 13.57 |
| ATOM | 3244 | CD2 | TYR | A | 423 | −13.942 | 20.572 | −30.991 | 1.00 | 12.74 |
| ATOM | 3245 | C | TYR | A | 423 | −16.217 | 17.673 | −27.658 | 1.00 | 12.12 |
| ATOM | 3246 | O | TYR | A | 423 | −17.430 | 17.622 | −27.323 | 1.00 | 12.13 |
| ATOM | 3247 | N | ALA | A | 424 | −15.385 | 16.623 | −27.539 | 1.00 | 10.99 |
| ATOM | 3248 | CA | ALA | A | 424 | −15.853 | 15.337 | −26.986 | 1.00 | 11.26 |
| ATOM | 3249 | CB | ALA | A | 424 | −14.717 | 14.294 | −26.952 | 1.00 | 11.73 |
| ATOM | 3250 | C | ALA | A | 424 | −16.411 | 15.535 | −25.588 | 1.00 | 10.93 |
| ATOM | 3251 | O | ALA | A | 424 | −17.465 | 14.974 | −25.246 | 1.00 | 11.68 |
| ATOM | 3252 | N | SER | A | 425 | −15.696 | 16.308 | −24.770 | 1.00 | 9.74 |
| ATOM | 3253 | CA | SER | A | 425 | −16.077 | 16.519 | −23.379 | 1.00 | 10.92 |
| ATOM | 3254 | CB | SER | A | 425 | −14.948 | 17.228 | −22.602 | 1.00 | 11.59 |
| ATOM | 3255 | OG | SER | A | 425 | −14.817 | 18.604 | −22.957 | 1.00 | 13.86 |
| ATOM | 3256 | C | SER | A | 425 | −17.402 | 17.291 | −23.219 | 1.00 | 11.65 |
| ATOM | 3257 | O | SER | A | 425 | −18.132 | 17.043 | −22.276 | 1.00 | 11.86 |
| ATOM | 3258 | N | PHE | A | 426 | −17.683 | 18.220 | −24.133 | 1.00 | 12.13 |
| ATOM | 3259 | CA | PHE | A | 426 | −18.983 | 18.891 | −24.154 | 1.00 | 12.95 |
| ATOM | 3260 | CB | PHE | A | 426 | −19.018 | 20.053 | −25.173 | 1.00 | 13.14 |
| ATOM | 3261 | CG | PHE | A | 426 | −20.410 | 20.575 | −25.391 | 1.00 | 15.93 |
| ATOM | 3262 | CD1 | PHE | A | 426 | −20.951 | 21.522 | −24.516 | 1.00 | 17.76 |
| ATOM | 3263 | CE1 | PHE | A | 426 | −22.279 | 21.980 | −24.688 | 1.00 | 17.49 |
| ATOM | 3264 | CZ | PHE | A | 426 | −23.064 | 21.455 | −25.716 | 1.00 | 17.22 |
| ATOM | 3265 | CE2 | PHE | A | 426 | −22.561 | 20.496 | −26.558 | 1.00 | 17.04 |
| ATOM | 3266 | CD2 | PHE | A | 426 | −21.225 | 20.045 | −26.396 | 1.00 | 17.29 |
| ATOM | 3267 | C | PHE | A | 426 | −20.079 | 17.878 | −24.502 | 1.00 | 13.31 |
| ATOM | 3268 | O | PHE | A | 426 | −21.120 | 17.827 | −23.850 | 1.00 | 13.38 |
| ATOM | 3269 | N | LEU | A | 427 | −19.834 | 17.077 | −25.539 | 1.00 | 13.23 |
| ATOM | 3270 | CA | LEU | A | 427 | −20.811 | 16.090 | −25.996 | 1.00 | 14.41 |
| ATOM | 3271 | CB | LEU | A | 427 | −20.339 | 15.410 | −27.291 | 1.00 | 14.32 |
| ATOM | 3272 | CG | LEU | A | 427 | −20.363 | 16.336 | −28.506 | 1.00 | 15.96 |
| ATOM | 3273 | CD1 | LEU | A | 427 | −19.661 | 15.639 | −29.689 | 1.00 | 18.66 |
| ATOM | 3274 | CD2 | LEU | A | 427 | −21.773 | 16.800 | −28.876 | 1.00 | 16.20 |
| ATOM | 3275 | C | LEU | A | 427 | −21.137 | 15.045 | −24.959 | 1.00 | 14.86 |
| ATOM | 3276 | O | LEU | A | 427 | −22.307 | 14.667 | −24.833 | 1.00 | 16.01 |
| ATOM | 3277 | N | THR | A | 428 | −20.130 | 14.551 | −24.235 | 1.00 | 13.81 |
| ATOM | 3278 | CA | THR | A | 428 | −20.397 | 13.544 | −23.196 | 1.00 | 13.67 |
| ATOM | 3279 | CB | THR | A | 428 | −19.134 | 12.732 | −22.745 | 1.00 | 12.87 |
| ATOM | 3280 | OG1 | THR | A | 428 | −18.127 | 13.597 | −22.185 | 1.00 | 12.33 |
| ATOM | 3281 | CG2 | THR | A | 428 | −18.533 | 11.902 | −23.923 | 1.00 | 13.77 |
| ATOM | 3282 | C | THR | A | 428 | −21.146 | 14.133 | −21.980 | 1.00 | 13.66 |
| ATOM | 3283 | O | THR | A | 428 | −22.102 | 13.517 | −21.478 | 1.00 | 15.09 |
| ATOM | 3284 | N | ALA | A | 429 | −20.738 | 15.312 | −21.524 | 1.00 | 13.72 |
| ATOM | 3285 | CA | ALA | A | 429 | −21.367 | 15.942 | −20.355 | 1.00 | 14.65 |
| ATOM | 3286 | CB | ALA | A | 429 | −20.704 | 17.295 | −20.036 | 1.00 | 14.11 |
| ATOM | 3287 | C | ALA | A | 429 | −22.852 | 16.158 | −20.643 | 1.00 | 15.47 |
| ATOM | 3288 | O | ALA | A | 429 | −23.704 | 15.908 | −19.783 | 1.00 | 15.97 |
| ATOM | 3289 | N | THR | A | 430 | −23.151 | 16.622 | −21.854 | 1.00 | 15.48 |
| ATOM | 3290 | CA | THR | A | 430 | −24.554 | 16.940 | −22.208 | 1.00 | 15.64 |
| ATOM | 3291 | CB | THR | A | 430 | −24.675 | 17.968 | −23.353 | 1.00 | 16.50 |
| ATOM | 3292 | OG1 | THR | A | 430 | −23.980 | 17.494 | −24.514 | 1.00 | 16.24 |
| ATOM | 3293 | CG2 | THR | A | 430 | −24.101 | 19.317 | −22.916 | 1.00 | 16.43 |
| ATOM | 3294 | C | THR | A | 430 | −25.401 | 15.690 | −22.463 | 1.00 | 15.41 |
| ATOM | 3295 | O | THR | A | 430 | −26.611 | 15.674 | −22.158 | 1.00 | 15.67 |
| ATOM | 3296 | N | ALA | A | 431 | −24.772 | 14.632 | −22.968 | 1.00 | 15.22 |
| ATOM | 3297 | CA | ALA | A | 431 | −25.437 | 13.311 | −23.049 | 1.00 | 16.26 |
| ATOM | 3298 | CB | ALA | A | 431 | −24.560 | 12.300 | −23.762 | 1.00 | 16.39 |
| ATOM | 3299 | C | ALA | A | 431 | −25.842 | 12.797 | −21.673 | 1.00 | 16.53 |
| ATOM | 3300 | O | ALA | A | 431 | −26.985 | 12.319 | −21.485 | 1.00 | 16.61 |
| ATOM | 3301 | N | ARG | A | 432 | −24.937 | 12.895 | −20.689 | 1.00 | 15.66 |
| ATOM | 3302 | CA | ARG | A | 432 | −25.256 | 12.410 | −19.342 | 1.00 | 15.64 |
| ATOM | 3303 | CB | ARG | A | 432 | −24.022 | 12.413 | −18.432 | 1.00 | 15.83 |
| ATOM | 3304 | CG | ARG | A | 432 | −22.862 | 11.574 | −18.994 | 1.00 | 14.85 |
| ATOM | 3305 | CD | ARG | A | 432 | −23.174 | 10.051 | −19.154 | 1.00 | 17.10 |
| ATOM | 3306 | NE | ARG | A | 432 | −21.958 | 9.472 | −19.708 | 1.00 | 18.67 |
| ATOM | 3307 | CZ | ARG | A | 432 | −21.766 | 9.225 | −21.003 | 1.00 | 20.81 |
| ATOM | 3308 | NH1 | ARG | A | 432 | −22.769 | 9.372 | −21.868 | 1.00 | 17.45 |
| ATOM | 3309 | NH2 | ARG | A | 432 | −20.576 | 8.781 | −21.427 | 1.00 | 19.90 |
| ATOM | 3310 | C | ARG | A | 432 | −26.375 | 13.235 | −18.719 | 1.00 | 16.42 |
| ATOM | 3311 | O | ARG | A | 432 | −27.256 | 12.685 | −18.030 | 1.00 | 17.47 |
| ATOM | 3312 | N | ARG | A | 433 | −26.371 | 14.535 | −18.996 | 1.00 | 16.54 |
| ATOM | 3313 | CA | ARG | A | 433 | −27.425 | 15.418 | −18.493 | 1.00 | 17.60 |
| ATOM | 3314 | CB | ARG | A | 433 | −27.204 | 16.852 | −18.960 | 1.00 | 16.74 |
| ATOM | 3315 | CG | ARG | A | 433 | −28.287 | 17.833 | −18.461 | 1.00 | 18.39 |
| ATOM | 3316 | CD | ARG | A | 433 | −27.931 | 19.239 | −18.866 | 1.00 | 20.79 |
| ATOM | 3317 | NE | ARG | A | 433 | −28.739 | 20.260 | −18.166 | 1.00 | 23.05 |
| ATOM | 3318 | CZ | ARG | A | 433 | −29.859 | 20.799 | −18.654 | 1.00 | 26.97 |
| ATOM | 3319 | NH1 | ARG | A | 433 | −30.333 | 20.404 | −19.837 | 1.00 | 24.94 |
| ATOM | 3320 | NH2 | ARG | A | 433 | −30.506 | 21.738 | −17.954 | 1.00 | 25.73 |
| ATOM | 3321 | C | ARG | A | 433 | −28.793 | 14.943 | −18.956 | 1.00 | 17.90 |
| ATOM | 3322 | O | ARG | A | 433 | −29.761 | 14.984 | −18.184 | 1.00 | 18.50 |
| ATOM | 3323 | N | ALA | A | 434 | −28.861 | 14.502 | −20.210 | 1.00 | 18.06 |
| ATOM | 3324 | CA | ALA | A | 434 | −30.103 | 13.993 | −20.806 | 1.00 | 19.10 |
| ATOM | 3325 | CB | ALA | A | 434 | −30.067 | 14.202 | −22.318 | 1.00 | 19.95 |
| ATOM | 3326 | C | ALA | A | 434 | −30.399 | 12.532 | −20.475 | 1.00 | 20.48 |
| ATOM | 3327 | O | ALA | A | 434 | −31.371 | 11.975 | −20.980 | 1.00 | 21.63 |
| ATOM | 3328 | N | GLY | A | 435 | −29.594 | 11.904 | −19.620 | 1.00 | 19.94 |
| ATOM | 3329 | CA | GLY | A | 435 | −29.865 | 10.531 | −19.182 | 1.00 | 21.09 |
| ATOM | 3330 | C | GLY | A | 435 | −29.415 | 9.475 | −20.188 | 1.00 | 21.57 |
| ATOM | 3331 | O | GLY | A | 435 | −29.847 | 8.304 | −20.130 | 1.00 | 21.64 |
| ATOM | 3332 | N | ILE | A | 436 | −28.529 | 9.873 | −21.100 | 1.00 | 20.44 |
| ATOM | 3333 | CA | ILE | A | 436 | −27.951 | 8.937 | −22.060 | 1.00 | 20.81 |
| ATOM | 3334 | CB | ILE | A | 436 | −27.753 | 9.601 | −23.447 | 1.00 | 20.75 |
| ATOM | 3335 | CG1 | ILE | A | 436 | −29.132 | 9.977 | −24.027 | 1.00 | 23.03 |
| ATOM | 3336 | CD1 | ILE | A | 436 | −29.103 | 11.031 | −25.128 | 1.00 | 26.34 |
| ATOM | 3337 | CG2 | ILE | A | 436 | −27.031 | 8.643 | −24.395 | 1.00 | 21.35 |
| ATOM | 3338 | C | ILE | A | 436 | −26.634 | 8.412 | −21.485 | 1.00 | 20.99 |
| ATOM | 3339 | O | ILE | A | 436 | −25.666 | 9.171 | −21.339 | 1.00 | 20.46 |
| ATOM | 3340 | N | VAL | A | 437 | −26.616 | 7.120 | −21.162 | 1.00 | 20.70 |
| ATOM | 3341 | CA | VAL | A | 437 | −25.465 | 6.479 | −20.517 | 1.00 | 21.39 |
| ATOM | 3342 | CB | VAL | A | 437 | −25.848 | 5.826 | −19.160 | 1.00 | 21.74 |
| ATOM | 3343 | CG1 | VAL | A | 437 | −26.340 | 6.911 | −18.205 | 1.00 | 22.12 |
| ATOM | 3344 | CG2 | VAL | A | 437 | −26.909 | 4.703 | −19.334 | 1.00 | 21.83 |
| ATOM | 3345 | C | VAL | A | 437 | −24.802 | 5.459 | −21.444 | 1.00 | 21.79 |
| ATOM | 3346 | O | VAL | A | 437 | −25.459 | 4.901 | −22.312 | 1.00 | 22.18 |
| ATOM | 3347 | N | PRO | A | 438 | −23.497 | 5.208 | −21.255 | 1.00 | 22.28 |
| ATOM | 3348 | CA | PRO | A | 438 | −22.837 | 4.291 | −22.181 | 1.00 | 22.83 |
| ATOM | 3349 | CB | PRO | A | 438 | −21.365 | 4.642 | −22.009 | 1.00 | 22.45 |
| ATOM | 3350 | CG | PRO | A | 438 | −21.248 | 5.054 | −20.578 | 1.00 | 23.88 |
| ATOM | 3351 | CD | PRO | A | 438 | −22.575 | 5.707 | −20.214 | 1.00 | 22.50 |
| ATOM | 3352 | C | PRO | A | 438 | −23.093 | 2.840 | −21.753 | 1.00 | 22.80 |
| ATOM | 3353 | O | PRO | A | 438 | −23.580 | 2.604 | −20.626 | 1.00 | 23.06 |
| ATOM | 3354 | N | PRO | A | 439 | −22.796 | 1.878 | −22.639 | 1.00 | 22.65 |
| ATOM | 3355 | CA | PRO | A | 439 | −22.911 | 0.452 | −22.283 | 1.00 | 22.08 |
| ATOM | 3356 | CB | PRO | A | 439 | −22.300 | −0.269 | −23.499 | 1.00 | 21.30 |
| ATOM | 3357 | CG | PRO | A | 439 | −22.526 | 0.664 | −24.618 | 1.00 | 22.81 |
| ATOM | 3358 | CD | PRO | A | 439 | −22.391 | 2.062 | −24.050 | 1.00 | 22.29 |
| ATOM | 3359 | C | PRO | A | 439 | −22.122 | 0.129 | −21.037 | 1.00 | 21.88 |
| ATOM | 3360 | O | PRO | A | 439 | −21.075 | 0.750 | −20.776 | 1.00 | 20.95 |
| ATOM | 3361 | N | SER | A | 440 | −22.628 | −0.818 | −20.253 | 1.00 | 22.34 |
| ATOM | 3362 | CA | SER | A | 440 | −21.932 | −1.273 | −19.060 | 1.00 | 23.42 |
| ATOM | 3363 | CB | SER | A | 440 | −22.818 | −2.195 | −18.224 | 1.00 | 24.01 |
| ATOM | 3364 | OG | SER | A | 440 | −23.805 | −1.412 | −17.566 | 1.00 | 26.78 |
| ATOM | 3365 | C | SER | A | 440 | −20.654 | −1.992 | −19.430 | 1.00 | 23.78 |
| ATOM | 3366 | O | SER | A | 440 | −20.540 | −2.554 | −20.522 | 1.00 | 23.97 |
| ATOM | 3367 | N | TRP | A | 441 | −19.681 | −1.929 | −18.536 | 1.00 | 24.15 |
| ATOM | 3368 | CA | TRP | A | 441 | −18.431 | −2.646 | −18.718 | 1.00 | 24.63 |
| ATOM | 3369 | CB | TRP | A | 441 | −17.255 | −1.679 | −18.819 | 1.00 | 22.62 |
| ATOM | 3370 | CG | TRP | A | 441 | −16.963 | −0.837 | −17.583 | 1.00 | 19.24 |
| ATOM | 3371 | CD1 | TRP | A | 441 | −17.409 | 0.432 | −17.339 | 1.00 | 16.95 |
| ATOM | 3372 | NE1 | TRP | A | 441 | −16.909 | 0.878 | −16.138 | 1.00 | 17.89 |
| ATOM | 3373 | CE2 | TRP | A | 441 | −16.111 | −0.098 | −15.595 | 1.00 | 17.88 |
| ATOM | 3374 | CD2 | TRP | A | 441 | −16.130 | −1.194 | −16.476 | 1.00 | 18.22 |
| ATOM | 3375 | CE3 | TRP | A | 441 | −15.377 | −2.338 | −16.149 | 1.00 | 19.64 |
| ATOM | 3376 | CZ3 | TRP | A | 441 | −14.658 | −2.355 | −14.961 | 1.00 | 18.61 |
| ATOM | 3377 | CH2 | TRP | A | 441 | −14.670 | −1.246 | −14.094 | 1.00 | 20.62 |
| ATOM | 3378 | CZ2 | TRP | A | 441 | −15.400 | −0.114 | −14.391 | 1.00 | 19.39 |
| ATOM | 3379 | C | TRP | A | 441 | −18.179 | −3.661 | −17.625 | 1.00 | 26.71 |
| ATOM | 3380 | O | TRP | A | 441 | −17.410 | −4.592 | −17.813 | 1.00 | 25.60 |
| ATOM | 3381 | N | ALA | A | 442 | −18.798 | −3.471 | −16.468 | 1.00 | 29.78 |
| ATOM | 3382 | CA | ALA | A | 442 | −18.442 | −4.292 | −15.330 | 1.00 | 33.94 |
| ATOM | 3383 | CB | ALA | A | 442 | −18.347 | −3.447 | −14.082 | 1.00 | 33.18 |
| ATOM | 3384 | C | ALA | A | 442 | −19.447 | −5.412 | −15.136 | 1.00 | 37.28 |
| ATOM | 3385 | O | ALA | A | 442 | −20.383 | −5.561 | −15.915 | 1.00 | 38.51 |
| ATOM | 3386 | N | ASN | A | 443 | −19.201 | −6.222 | −14.116 | 1.00 | 41.45 |
| ATOM | 3387 | CA | ASN | A | 443 | −20.226 | −7.030 | −13.467 | 1.00 | 45.10 |
| ATOM | 3388 | CB | ASN | A | 443 | −20.135 | −8.490 | −13.914 | 1.00 | 45.75 |
| ATOM | 3389 | CG | ASN | A | 443 | −18.815 | −9.129 | −13.531 | 1.00 | 48.14 |
| ATOM | 3390 | OD1 | ASN | A | 443 | −18.620 | −9.524 | −12.380 | 1.00 | 50.51 |
| ATOM | 3391 | ND2 | ASN | A | 443 | −17.888 | −9.212 | −14.492 | 1.00 | 50.01 |
| ATOM | 3392 | C | ASN | A | 443 | −19.946 | −6.892 | −11.972 | 1.00 | 46.93 |
| ATOM | 3393 | O | ASN | A | 443 | −18.878 | −6.396 | −11.580 | 1.00 | 47.13 |
| ATOM | 3394 | N | SER | A | 444 | −20.873 | −7.343 | −11.135 | 1.00 | 49.21 |
| ATOM | 3395 | CA | SER | A | 444 | −20.719 | −7.201 | −9.684 | 1.00 | 51.08 |
| ATOM | 3396 | CB | SER | A | 444 | −21.713 | −8.096 | −8.936 | 1.00 | 51.21 |
| ATOM | 3397 | OG | SER | A | 444 | −21.738 | −7.735 | −7.563 | 1.00 | 52.90 |
| ATOM | 3398 | C | SER | A | 444 | −19.291 | −7.452 | −9.165 | 1.00 | 51.89 |
| ATOM | 3399 | O | SER | A | 444 | −18.743 | −6.610 | −8.433 | 1.00 | 52.46 |
| ATOM | 3400 | N | SER | A | 445 | −18.700 | −8.588 | −9.558 | 1.00 | 52.45 |
| ATOM | 3401 | CA | SER | A | 445 | −17.400 | −9.030 | −9.029 | 1.00 | 52.98 |
| ATOM | 3402 | CB | SER | A | 445 | −17.138 | −10.497 | −9.385 | 1.00 | 53.12 |
| ATOM | 3403 | OG | SER | A | 445 | −16.901 | −10.650 | −10.775 | 1.00 | 54.45 |
| ATOM | 3404 | C | SER | A | 445 | −16.202 | −8.159 | −9.449 | 1.00 | 52.99 |
| ATOM | 3405 | O | SER | A | 445 | −15.184 | −8.112 | −8.738 | 1.00 | 53.28 |
| ATOM | 3406 | N | ALA | A | 446 | −16.328 | −7.472 | −10.588 | 1.00 | 52.67 |
| ATOM | 3407 | CA | ALA | A | 446 | −15.301 | −6.536 | −11.063 | 1.00 | 52.20 |
| ATOM | 3408 | CB | ALA | A | 446 | −15.695 | −5.960 | −12.425 | 1.00 | 52.45 |
| ATOM | 3409 | C | ALA | A | 446 | −14.996 | −5.406 | −10.059 | 1.00 | 51.92 |
| ATOM | 3410 | O | ALA | A | 446 | −14.144 | −4.553 | −10.322 | 1.00 | 51.77 |
| ATOM | 3411 | N | SER | A | 447 | −15.696 | −5.413 | −8.919 | 1.00 | 51.23 |
| ATOM | 3412 | CA | SER | A | 447 | −15.471 | −4.453 | −7.827 | 1.00 | 50.73 |
| ATOM | 3413 | CB | SER | A | 447 | −16.769 | −3.706 | −7.495 | 1.00 | 50.70 |
| ATOM | 3414 | OG | SER | A | 447 | −17.765 | −4.605 | −7.021 | 1.00 | 50.91 |
| ATOM | 3415 | C | SER | A | 447 | −14.886 | −5.072 | −6.537 | 1.00 | 50.31 |
| ATOM | 3416 | O | SER | A | 447 | −14.604 | −4.345 | −5.580 | 1.00 | 50.16 |
| ATOM | 3417 | N | THR | A | 448 | −14.704 | −6.394 | −6.508 | 1.00 | 49.55 |
| ATOM | 3418 | CA | THR | A | 448 | −14.165 | −7.070 | −5.314 | 1.00 | 49.05 |
| ATOM | 3419 | CB | THR | A | 448 | −14.579 | −8.561 | −5.233 | 1.00 | 49.15 |
| ATOM | 3420 | OG1 | THR | A | 448 | −14.076 | −9.255 | −6.378 | 1.00 | 49.97 |
| ATOM | 3421 | CG2 | THR | A | 448 | −16.096 | −8.705 | −5.180 | 1.00 | 49.11 |
| ATOM | 3422 | C | THR | A | 448 | −12.641 | −6.958 | −5.215 | 1.00 | 48.34 |
| ATOM | 3423 | O | THR | A | 448 | −11.911 | −7.325 | −6.135 | 1.00 | 47.84 |
| ATOM | 3424 | N | ILE | A | 449 | −12.174 | −6.444 | −4.084 | 1.00 | 47.93 |
| ATOM | 3425 | CA | ILE | A | 449 | −10.760 | −6.150 | −3.891 | 1.00 | 47.42 |
| ATOM | 3426 | CB | ILE | A | 449 | −10.577 | −4.798 | −3.142 | 1.00 | 47.53 |
| ATOM | 3427 | CG1 | ILE | A | 449 | −11.346 | −3.680 | −3.863 | 1.00 | 46.60 |
| ATOM | 3428 | CD1 | ILE | A | 449 | −11.727 | −2.523 | −2.981 | 1.00 | 45.49 |
| ATOM | 3429 | CG2 | ILE | A | 449 | −9.097 | −4.438 | −2.999 | 1.00 | 46.84 |
| ATOM | 3430 | C | ILE | A | 449 | −10.104 | −7.299 | −3.124 | 1.00 | 47.45 |
| ATOM | 3431 | O | ILE | A | 449 | −10.606 | −7.688 | −2.067 | 1.00 | 47.66 |
| ATOM | 3432 | N | PRO | A | 450 | −8.993 | −7.857 | −3.663 | 1.00 | 47.28 |
| ATOM | 3433 | CA | PRO | A | 450 | −8.202 | −8.926 | −3.036 | 1.00 | 47.25 |
| ATOM | 3434 | CB | PRO | A | 450 | −6.982 | −9.040 | −3.946 | 1.00 | 47.03 |
| ATOM | 3435 | CG | PRO | A | 450 | −7.430 | −8.535 | −5.237 | 1.00 | 47.02 |
| ATOM | 3436 | CD | PRO | A | 450 | −8.431 | −7.470 | −4.969 | 1.00 | 47.07 |
| ATOM | 3437 | C | PRO | A | 450 | −7.731 | −8.562 | −1.639 | 1.00 | 47.48 |
| ATOM | 3438 | O | PRO | A | 450 | −7.608 | −7.377 | −1.322 | 1.00 | 47.43 |
| ATOM | 3439 | N | SER | A | 451 | −7.452 | −9.587 | −0.832 | 1.00 | 47.94 |
| ATOM | 3440 | CA | SER | A | 451 | −7.020 | −9.436 | 0.568 | 1.00 | 48.19 |
| ATOM | 3441 | CB | SER | A | 451 | −7.017 | −10.801 | 1.277 | 1.00 | 48.52 |
| ATOM | 3442 | OG | SER | A | 451 | −8.297 | −11.414 | 1.235 | 1.00 | 49.64 |
| ATOM | 3443 | C | SER | A | 451 | −5.641 | −8.799 | 0.701 | 1.00 | 47.83 |
| ATOM | 3444 | O | SER | A | 451 | −5.415 | −7.963 | 1.575 | 1.00 | 48.39 |
| ATOM | 3445 | N | THR | A | 452 | −4.715 | −9.212 | −0.158 | 1.00 | 47.14 |
| ATOM | 3446 | CA | THR | A | 452 | −3.379 | −8.613 | −0.211 | 1.00 | 46.42 |
| ATOM | 3447 | CB | THR | A | 452 | −2.323 | −9.540 | 0.434 | 1.00 | 46.66 |
| ATOM | 3448 | OG1 | THR | A | 452 | −2.518 | −10.887 | −0.032 | 1.00 | 48.38 |
| ATOM | 3449 | CG2 | THR | A | 452 | −2.446 | −9.514 | 1.962 | 1.00 | 47.77 |
| ATOM | 3450 | C | THR | A | 452 | −3.011 | −8.323 | −1.673 | 1.00 | 44.81 |
| ATOM | 3451 | O | THR | A | 452 | −3.348 | −9.107 | −2.558 | 1.00 | 44.97 |
| ATOM | 3452 | N | CYS | A | 453 | −2.363 | −7.187 | −1.931 | 1.00 | 43.37 |
| ATOM | 3453 | CA | CYS | A | 453 | −1.971 | −6.854 | −3.306 | 1.00 | 41.40 |
| ATOM | 3454 | CB | CYS | A | 453 | −1.918 | −5.339 | −3.574 | 1.00 | 41.01 |
| ATOM | 3455 | SG | CYS | A | 453 | −3.187 | −4.199 | −2.908 | 1.00 | 40.58 |
| ATOM | 3456 | C | CYS | A | 453 | −0.591 | −7.408 | −3.602 | 1.00 | 40.33 |
| ATOM | 3457 | O | CYS | A | 453 | 0.293 | −7.370 | −2.753 | 1.00 | 39.88 |
| ATOM | 3458 | N | SER | A | 454 | −0.405 | −7.911 | −4.812 | 1.00 | 39.25 |
| ATOM | 3459 | CA | SER | A | 454 | 0.937 | −8.142 | −5.336 | 1.00 | 38.68 |
| ATOM | 3460 | CB | SER | A | 454 | 1.222 | −9.638 | −5.484 | 1.00 | 38.56 |
| ATOM | 3461 | OG | SER | A | 454 | 0.276 | −10.251 | −6.349 | 1.00 | 40.44 |
| ATOM | 3462 | C | SER | A | 454 | 1.047 | −7.450 | −6.690 | 1.00 | 37.78 |
| ATOM | 3463 | O | SER | A | 454 | 0.030 | −7.187 | −7.347 | 1.00 | 36.71 |
| ATOM | 3464 | N | GLY | A | 455 | 2.275 | −7.175 | −7.111 | 1.00 | 37.06 |
| ATOM | 3465 | CA | GLY | A | 455 | 2.514 | −6.613 | −8.431 | 1.00 | 36.79 |
| ATOM | 3466 | C | GLY | A | 455 | 2.493 | −7.658 | −9.539 | 1.00 | 36.18 |
| ATOM | 3467 | O | GLY | A | 455 | 3.410 | −7.708 | −10.367 | 1.00 | 36.73 |
| ATOM | 3468 | N | ALA | A | 456 | 1.445 | −8.480 | −9.562 | 1.00 | 35.26 |
| ATOM | 3469 | CA | ALA | A | 456 | 1.321 | −9.584 | −10.512 | 1.00 | 34.46 |
| ATOM | 3470 | CB | ALA | A | 456 | 0.125 | −10.454 | −10.158 | 1.00 | 34.50 |
| ATOM | 3471 | C | ALA | A | 456 | 1.195 | −9.111 | −11.963 | 1.00 | 34.16 |
| ATOM | 3472 | O | ALA | A | 456 | 0.283 | −8.353 | −12.301 | 1.00 | 33.56 |
| ATOM | 3473 | N | SER | A | 457 | 2.097 | −9.588 | −12.817 | 1.00 | 33.11 |
| ATOM | 3474 | CA | SER | A | 457 | 2.013 | −9.302 | −14.241 | 1.00 | 32.60 |
| ATOM | 3475 | CB | SER | A | 457 | 3.022 | −8.219 | −14.635 | 1.00 | 32.50 |
| ATOM | 3476 | OG | SER | A | 457 | 4.352 | −8.691 | −14.519 | 1.00 | 33.28 |
| ATOM | 3477 | C | SER | A | 457 | 2.228 | −10.575 | −15.044 | 1.00 | 32.31 |
| ATOM | 3478 | O | SER | A | 457 | 2.641 | −11.605 | −14.494 | 1.00 | 32.32 |
| ATOM | 3479 | N | VAL | A | 458 | 1.908 | −10.511 | −16.330 | 1.00 | 31.12 |
| ATOM | 3480 | CA | VAL | A | 458 | 2.063 | −11.629 | −17.246 | 1.00 | 31.09 |
| ATOM | 3481 | CB | VAL | A | 458 | 0.682 | −12.199 | −17.659 | 1.00 | 31.00 |
| ATOM | 3482 | CG1 | VAL | A | 458 | 0.806 | −13.173 | −18.830 | 1.00 | 30.72 |
| ATOM | 3483 | CG2 | VAL | A | 458 | −0.014 | −12.847 | −16.459 | 1.00 | 31.08 |
| ATOM | 3484 | C | VAL | A | 458 | 2.817 | −11.144 | −18.480 | 1.00 | 30.88 |
| ATOM | 3485 | O | VAL | A | 458 | 2.401 | −10.177 | −19.126 | 1.00 | 29.70 |
| ATOM | 3486 | N | VAL | A | 459 | 3.924 | −11.811 | −18.805 | 1.00 | 30.81 |
| ATOM | 3487 | CA | VAL | A | 459 | 4.643 | −11.525 | −20.051 | 1.00 | 30.95 |
| ATOM | 3488 | CB | VAL | A | 459 | 6.046 | −12.172 | −20.071 | 1.00 | 31.34 |
| ATOM | 3489 | CG1 | VAL | A | 459 | 6.664 | −12.102 | −21.492 | 1.00 | 30.45 |
| ATOM | 3490 | CG2 | VAL | A | 459 | 6.947 | −11.522 | −19.030 | 1.00 | 32.03 |
| ATOM | 3491 | C | VAL | A | 459 | 3.805 | −12.032 | −21.227 | 1.00 | 31.37 |
| ATOM | 3492 | O | VAL | A | 459 | 3.443 | −13.214 | −21.288 | 1.00 | 31.84 |
| ATOM | 3493 | N | GLY | A | 460 | 3.480 | −11.137 | −22.154 | 1.00 | 30.71 |
| ATOM | 3494 | CA | GLY | A | 460 | 2.596 | −11.495 | −23.258 | 1.00 | 30.77 |
| ATOM | 3495 | C | GLY | A | 460 | 3.349 | −11.799 | −24.536 | 1.00 | 30.66 |
| ATOM | 3496 | O | GLY | A | 460 | 4.585 | −11.773 | −24.582 | 1.00 | 30.90 |
| ATOM | 3497 | N | SER | A | 461 | 2.606 | −12.094 | −25.584 | 1.00 | 30.44 |
| ATOM | 3498 | CA | SER | A | 461 | 3.219 | −12.227 | −26.877 | 1.00 | 31.06 |
| ATOM | 3499 | CB | SER | A | 461 | 3.301 | −13.695 | −27.308 | 1.00 | 31.44 |
| ATOM | 3500 | OG | SER | A | 461 | 2.018 | −14.278 | −27.419 | 1.00 | 34.77 |
| ATOM | 3501 | C | SER | A | 461 | 2.463 | −11.357 | −27.864 | 1.00 | 30.21 |
| ATOM | 3502 | O | SER | A | 461 | 1.246 | −11.156 | −27.736 | 1.00 | 30.87 |
| ATOM | 3503 | N | TYR | A | 462 | 3.192 | −10.822 | −28.836 | 1.00 | 28.84 |
| ATOM | 3504 | CA | TYR | A | 462 | 2.651 | −9.797 | −29.712 | 1.00 | 28.13 |
| ATOM | 3505 | CB | TYR | A | 462 | 3.365 | −8.471 | −29.426 | 1.00 | 26.74 |
| ATOM | 3506 | CG | TYR | A | 462 | 3.264 | −8.098 | −27.976 | 1.00 | 25.28 |
| ATOM | 3507 | CD1 | TYR | A | 462 | 2.184 | −7.335 | −27.508 | 1.00 | 23.75 |
| ATOM | 3508 | CE1 | TYR | A | 462 | 2.066 | −7.022 | −26.162 | 1.00 | 23.41 |
| ATOM | 3509 | CZ | TYR | A | 462 | 3.030 | −7.458 | −25.268 | 1.00 | 23.32 |
| ATOM | 3510 | OH | TYR | A | 462 | 2.907 | −7.155 | −23.941 | 1.00 | 23.50 |
| ATOM | 3511 | CE2 | TYR | A | 462 | 4.113 | −8.226 | −25.698 | 1.00 | 22.57 |
| ATOM | 3512 | CD2 | TYR | A | 462 | 4.223 | −8.537 | −27.050 | 1.00 | 24.71 |
| ATOM | 3513 | C | TYR | A | 462 | 2.844 | −10.191 | −31.152 | 1.00 | 28.83 |
| ATOM | 3514 | O | TYR | A | 462 | 3.898 | −10.697 | −31.529 | 1.00 | 29.33 |
| ATOM | 3515 | N | SER | A | 463 | 1.828 | −9.973 | −31.961 | 1.00 | 29.66 |
| ATOM | 3516 | CA | SER | A | 463 | 1.970 | −10.202 | −33.388 | 1.00 | 30.77 |
| ATOM | 3517 | CB | SER | A | 463 | 1.424 | −11.574 | −33.784 | 1.00 | 30.99 |
| ATOM | 3518 | OG | SER | A | 463 | 0.168 | −11.815 | −33.192 | 1.00 | 33.22 |
| ATOM | 3519 | C | SER | A | 463 | 1.311 | −9.082 | −34.170 | 1.00 | 31.28 |
| ATOM | 3520 | O | SER | A | 463 | 0.329 | −8.481 | −33.723 | 1.00 | 30.40 |
| ATOM | 3521 | N | ARG | A | 464 | 1.886 | −8.789 | −35.330 | 1.00 | 31.94 |
| ATOM | 3522 | CA | ARG | A | 464 | 1.377 | −7.775 | −36.225 | 1.00 | 32.97 |
| ATOM | 3523 | CB | ARG | A | 464 | 2.362 | −7.620 | −37.385 | 1.00 | 33.68 |
| ATOM | 3524 | CG | ARG | A | 464 | 2.353 | −6.274 | −38.061 | 1.00 | 36.94 |
| ATOM | 3525 | CD | ARG | A | 464 | 3.502 | −6.206 | −39.088 | 1.00 | 42.45 |
| ATOM | 3526 | NE | ARG | A | 464 | 4.794 | −6.065 | −38.415 | 1.00 | 45.53 |
| ATOM | 3527 | CZ | ARG | A | 464 | 5.416 | −4.903 | −38.227 | 1.00 | 47.50 |
| ATOM | 3528 | NH1 | ARG | A | 464 | 4.882 | −3.775 | −38.688 | 1.00 | 49.50 |
| ATOM | 3529 | NH2 | ARG | A | 464 | 6.580 | −4.863 | −37.592 | 1.00 | 48.59 |
| ATOM | 3530 | C | ARG | A | 464 | −0.017 | −8.171 | −36.741 | 1.00 | 33.03 |
| ATOM | 3531 | O | ARG | A | 464 | −0.166 | −9.228 | −37.358 | 1.00 | 33.00 |
| ATOM | 3532 | N | PRO | A | 465 | −1.053 | −7.333 | −36.479 | 1.00 | 32.72 |
| ATOM | 3533 | CA | PRO | A | 465 | −2.344 | −7.593 | −37.131 | 1.00 | 32.69 |
| ATOM | 3534 | CB | PRO | A | 465 | −3.274 | −6.504 | −36.558 | 1.00 | 32.50 |
| ATOM | 3535 | CG | PRO | A | 465 | −2.581 | −5.981 | −35.345 | 1.00 | 32.47 |
| ATOM | 3536 | CD | PRO | A | 465 | −1.102 | −6.134 | −35.618 | 1.00 | 32.83 |
| ATOM | 3537 | C | PRO | A | 465 | −2.189 | −7.421 | −38.642 | 1.00 | 33.30 |
| ATOM | 3538 | O | PRO | A | 465 | −1.332 | −6.661 | −39.097 | 1.00 | 33.20 |
| ATOM | 3539 | N | THR | A | 466 | −2.990 | −8.136 | −39.412 | 1.00 | 34.26 |
| ATOM | 3540 | CA | THR | A | 466 | −2.810 | −8.131 | −40.855 | 1.00 | 35.62 |
| ATOM | 3541 | CB | THR | A | 466 | −2.264 | −9.486 | −41.370 | 1.00 | 35.33 |
| ATOM | 3542 | OG1 | THR | A | 466 | −3.225 | −10.512 | −41.136 | 1.00 | 36.81 |
| ATOM | 3543 | CG2 | THR | A | 466 | −0.965 | −9.848 | −40.656 | 1.00 | 35.91 |
| ATOM | 3544 | C | THR | A | 466 | −4.076 | −7.711 | −41.600 | 1.00 | 36.15 |
| ATOM | 3545 | O | THR | A | 466 | −3.983 | −7.077 | −42.648 | 1.00 | 36.93 |
| ATOM | 3546 | N | ALA | A | 467 | −5.242 | −8.051 | −41.048 | 1.00 | 36.82 |
| ATOM | 3547 | CA | ALA | A | 467 | −6.540 | −7.648 | −41.609 | 1.00 | 37.30 |
| ATOM | 3548 | CB | ALA | A | 467 | −7.663 | −8.403 | −40.930 | 1.00 | 37.04 |
| ATOM | 3549 | C | ALA | A | 467 | −6.767 | −6.136 | −41.509 | 1.00 | 38.13 |
| ATOM | 3550 | O | ALA | A | 467 | −6.715 | −5.556 | −40.417 | 1.00 | 37.96 |
| ATOM | 3551 | N | THR | A | 468 | −7.011 | −5.502 | −42.653 | 1.00 | 38.66 |
| ATOM | 3552 | CA | THR | A | 468 | −7.146 | −4.050 | −42.702 | 1.00 | 39.18 |
| ATOM | 3553 | CB | THR | A | 468 | −5.970 | −3.406 | −43.428 | 1.00 | 39.43 |
| ATOM | 3554 | OG1 | THR | A | 468 | −5.955 | −3.879 | −44.778 | 1.00 | 40.06 |
| ATOM | 3555 | CG2 | THR | A | 468 | −4.637 | −3.717 | −42.734 | 1.00 | 39.67 |
| ATOM | 3556 | C | THR | A | 468 | −8.405 | −3.591 | −43.427 | 1.00 | 39.33 |
| ATOM | 3557 | O | THR | A | 468 | −8.468 | −2.451 | −43.890 | 1.00 | 39.79 |
| ATOM | 3558 | N | SER | A | 469 | −9.403 | −4.457 | −43.529 | 1.00 | 39.12 |
| ATOM | 3559 | CA | SER | A | 469 | −10.651 | −4.065 | −44.176 | 1.00 | 39.41 |
| ATOM | 3560 | CB | SER | A | 469 | −10.624 | −4.369 | −45.684 | 1.00 | 39.77 |
| ATOM | 3561 | OG | SER | A | 469 | −10.476 | −5.763 | −45.916 | 1.00 | 41.02 |
| ATOM | 3562 | C | SER | A | 469 | −11.850 | −4.732 | −43.537 | 1.00 | 38.54 |
| ATOM | 3563 | O | SER | A | 469 | −11.771 | −5.856 | −43.046 | 1.00 | 38.85 |
| ATOM | 3564 | N | PHE | A | 470 | −12.962 | −4.014 | −43.558 | 1.00 | 38.00 |
| ATOM | 3565 | CA | PHE | A | 470 | −14.216 | −4.507 | −43.031 | 1.00 | 37.29 |
| ATOM | 3566 | CB | PHE | A | 470 | −14.880 | −3.406 | −42.220 | 1.00 | 36.87 |
| ATOM | 3567 | CG | PHE | A | 470 | −14.277 | −3.212 | −40.865 | 1.00 | 35.43 |
| ATOM | 3568 | CD1 | PHE | A | 470 | −13.146 | −2.428 | −40.696 | 1.00 | 35.15 |
| ATOM | 3569 | CE1 | PHE | A | 470 | −12.589 | −2.252 | −39.437 | 1.00 | 33.73 |
| ATOM | 3570 | CZ | PHE | A | 470 | −13.159 | −2.852 | −38.332 | 1.00 | 35.04 |
| ATOM | 3571 | CE2 | PHE | A | 470 | −14.292 | −3.638 | −38.479 | 1.00 | 36.26 |
| ATOM | 3572 | CD2 | PHE | A | 470 | −14.844 | −3.816 | −39.751 | 1.00 | 35.71 |
| ATOM | 3573 | C | PHE | A | 470 | −15.128 | −4.923 | −44.181 | 1.00 | 37.34 |
| ATOM | 3574 | O | PHE | A | 470 | −15.059 | −4.337 | −45.258 | 1.00 | 37.21 |
| ATOM | 3575 | N | PRO | A | 471 | −15.987 | −5.931 | −43.959 | 1.00 | 37.46 |
| ATOM | 3576 | CA | PRO | A | 471 | −16.983 | −6.243 | −44.985 | 1.00 | 38.04 |
| ATOM | 3577 | CB | PRO | A | 471 | −17.790 | −7.383 | −44.361 | 1.00 | 37.60 |
| ATOM | 3578 | CG | PRO | A | 471 | −16.877 | −7.986 | −43.337 | 1.00 | 38.09 |
| ATOM | 3579 | CD | PRO | A | 471 | −16.093 | −6.828 | −42.795 | 1.00 | 37.31 |
| ATOM | 3580 | C | PRO | A | 471 | −17.879 | −5.033 | −45.231 | 1.00 | 38.96 |
| ATOM | 3581 | O | PRO | A | 471 | −18.108 | −4.245 | −44.306 | 1.00 | 38.78 |
| ATOM | 3582 | N | PRO | A | 472 | −18.378 | −4.869 | −46.471 | 1.00 | 39.78 |
| ATOM | 3583 | CA | PRO | A | 472 | −19.238 | −3.723 | −46.771 | 1.00 | 39.87 |
| ATOM | 3584 | CB | PRO | A | 472 | −19.378 | −3.780 | −48.293 | 1.00 | 40.09 |
| ATOM | 3585 | CG | PRO | A | 472 | −19.171 | −5.225 | −48.635 | 1.00 | 40.28 |
| ATOM | 3586 | CD | PRO | A | 472 | −18.171 | −5.740 | −47.649 | 1.00 | 40.02 |
| ATOM | 3587 | C | PRO | A | 472 | −20.604 | −3.864 | −46.114 | 1.00 | 39.75 |
| ATOM | 3588 | O | PRO | A | 472 | −21.017 | −4.980 | −45.798 | 1.00 | 40.10 |
| ATOM | 3589 | N | SER | A | 473 | −21.268 | −2.734 | −45.881 | 1.00 | 39.71 |
| ATOM | 3590 | CA | SER | A | 473 | −22.675 | −2.696 | −45.467 | 1.00 | 39.61 |
| ATOM | 3591 | CB | SER | A | 473 | −23.571 | −3.070 | −46.651 | 1.00 | 40.16 |
| ATOM | 3592 | OG | SER | A | 473 | −23.468 | −2.074 | −47.658 | 1.00 | 42.55 |
| ATOM | 3593 | C | SER | A | 473 | −23.043 | −3.509 | −44.221 | 1.00 | 38.94 |
| ATOM | 3594 | O | SER | A | 473 | −24.041 | −4.258 | −44.210 | 1.00 | 39.07 |
| ATOM | 3595 | N | GLN | A | 474 | −22.257 | −3.340 | −43.159 | 1.00 | 37.75 |
| ATOM | 3596 | CA | GLN | A | 474 | −22.558 | −3.993 | −41.888 | 1.00 | 36.60 |
| ATOM | 3597 | CB | GLN | A | 474 | −21.291 | −4.205 | −41.057 | 1.00 | 36.41 |
| ATOM | 3598 | CG | GLN | A | 474 | −20.331 | −5.169 | −41.732 | 1.00 | 36.24 |
| ATOM | 3599 | CD | GLN | A | 474 | −19.295 | −5.757 | −40.795 | 1.00 | 36.45 |
| ATOM | 3600 | OE1 | GLN | A | 474 | −18.478 | −5.040 | −40.211 | 1.00 | 35.30 |
| ATOM | 3601 | NE2 | GLN | A | 474 | −19.304 | −7.077 | −40.671 | 1.00 | 36.85 |
| ATOM | 3602 | C | GLN | A | 474 | −23.620 | −3.191 | −41.149 | 1.00 | 36.40 |
| ATOM | 3603 | O | GLN | A | 474 | −23.329 | −2.444 | −40.208 | 1.00 | 35.99 |
| ATOM | 3604 | N | THR | A | 475 | −24.859 | −3.350 | −41.616 | 1.00 | 35.59 |
| ATOM | 3605 | CA | THR | A | 475 | −26.012 | −2.595 | −41.148 | 1.00 | 35.20 |
| ATOM | 3606 | CB | THR | A | 475 | −27.051 | −2.478 | −42.287 | 1.00 | 35.73 |
| ATOM | 3607 | OG1 | THR | A | 475 | −27.120 | −3.737 | −42.959 | 1.00 | 36.85 |
| ATOM | 3608 | CG2 | THR | A | 475 | −26.642 | −1.418 | −43.310 | 1.00 | 34.51 |
| ATOM | 3609 | C | THR | A | 475 | −26.635 | −3.256 | −39.910 | 1.00 | 35.16 |
| ATOM | 3610 | O | THR | A | 475 | −26.363 | −4.420 | −39.622 | 1.00 | 34.47 |
| ATOM | 3611 | N | PRO | A | 476 | −27.453 | −2.510 | −39.148 | 1.00 | 35.36 |
| ATOM | 3612 | CA | PRO | A | 476 | −27.990 | −3.111 | −37.923 | 1.00 | 36.40 |
| ATOM | 3613 | CB | PRO | A | 476 | −28.567 | −1.910 | −37.167 | 1.00 | 36.04 |
| ATOM | 3614 | CG | PRO | A | 476 | −28.890 | −0.912 | −38.230 | 1.00 | 35.98 |
| ATOM | 3615 | CD | PRO | A | 476 | −27.907 | −1.119 | −39.339 | 1.00 | 35.70 |
| ATOM | 3616 | C | PRO | A | 476 | −29.085 | −4.171 | −38.158 | 1.00 | 37.45 |
| ATOM | 3617 | O | PRO | A | 476 | −29.654 | −4.244 | −39.254 | 1.00 | 36.95 |
| ATOM | 3618 | N | LYS | A | 477 | −29.342 | −4.984 | −37.133 | 1.00 | 39.01 |
| ATOM | 3619 | CA | LYS | A | 477 | −30.472 | −5.911 | −37.111 | 1.00 | 41.20 |
| ATOM | 3620 | CB | LYS | A | 477 | −30.541 | −6.641 | −35.774 | 1.00 | 41.46 |
| ATOM | 3621 | CG | LYS | A | 477 | −29.665 | −7.850 | −35.604 | 1.00 | 41.87 |
| ATOM | 3622 | CD | LYS | A | 477 | −29.939 | −8.517 | −34.237 | 1.00 | 42.36 |
| ATOM | 3623 | CE | LYS | A | 477 | −29.996 | −7.497 | −33.076 | 1.00 | 44.55 |
| ATOM | 3624 | NZ | LYS | A | 477 | −29.705 | −8.110 | −31.718 | 1.00 | 44.56 |
| ATOM | 3625 | C | LYS | A | 477 | −31.766 | −5.118 | −37.230 | 1.00 | 42.27 |
| ATOM | 3626 | O | LYS | A | 477 | −31.818 | −3.960 | −36.798 | 1.00 | 42.18 |
| ATOM | 3627 | N | PRO | A | 478 | −32.831 | −5.743 | −37.780 | 1.00 | 43.46 |
| ATOM | 3628 | CA | PRO | A | 478 | −34.150 | −5.106 | −37.669 | 1.00 | 44.02 |
| ATOM | 3629 | CB | PRO | A | 478 | −35.106 | −6.144 | −38.267 | 1.00 | 43.89 |
| ATOM | 3630 | CG | PRO | A | 478 | −34.255 | −7.033 | −39.105 | 1.00 | 44.08 |
| ATOM | 3631 | CD | PRO | A | 478 | −32.885 | −7.034 | −38.493 | 1.00 | 43.41 |
| ATOM | 3632 | C | PRO | A | 478 | −34.480 | −4.892 | −36.194 | 1.00 | 44.52 |
| ATOM | 3633 | O | PRO | A | 478 | −34.197 | −5.769 | −35.364 | 1.00 | 44.73 |
| ATOM | 3634 | N | GLY | A | 479 | −35.043 | −3.728 | −35.874 | 1.00 | 45.20 |
| ATOM | 3635 | CA | GLY | A | 479 | −35.421 | −3.395 | −34.494 | 1.00 | 45.90 |
| ATOM | 3636 | C | GLY | A | 479 | −34.386 | −2.601 | −33.711 | 1.00 | 46.37 |
| ATOM | 3637 | O | GLY | A | 479 | −34.576 | −2.331 | −32.520 | 1.00 | 46.98 |
| ATOM | 3638 | N | VAL | A | 480 | −33.282 | −2.244 | −34.367 | 1.00 | 46.28 |
| ATOM | 3639 | CA | VAL | A | 480 | −32.261 | −1.383 | −33.760 | 1.00 | 46.15 |
| ATOM | 3640 | CB | VAL | A | 480 | −30.820 | −1.863 | −34.121 | 1.00 | 46.20 |
| ATOM | 3641 | CG1 | VAL | A | 480 | −29.755 | −0.899 | −33.584 | 1.00 | 45.85 |
| ATOM | 3642 | CG2 | VAL | A | 480 | −30.569 | −3.281 | −33.603 | 1.00 | 46.25 |
| ATOM | 3643 | C | VAL | A | 480 | −32.498 | 0.046 | −34.260 | 1.00 | 45.93 |
| ATOM | 3644 | O | VAL | A | 480 | −32.673 | 0.240 | −35.465 | 1.00 | 46.15 |
| ATOM | 3645 | N | PRO | A | 481 | −32.534 | 1.049 | −33.344 | 1.00 | 45.70 |
| ATOM | 3646 | CA | PRO | A | 481 | −32.648 | 2.443 | −33.804 | 1.00 | 45.36 |
| ATOM | 3647 | CB | PRO | A | 481 | −32.388 | 3.266 | −32.542 | 1.00 | 45.39 |
| ATOM | 3648 | CG | PRO | A | 481 | −32.778 | 2.375 | −31.427 | 1.00 | 45.52 |
| ATOM | 3649 | CD | PRO | A | 481 | −32.481 | 0.962 | −31.873 | 1.00 | 45.83 |
| ATOM | 3650 | C | PRO | A | 481 | −31.609 | 2.762 | −34.877 | 1.00 | 45.08 |
| ATOM | 3651 | O | PRO | A | 481 | −30.405 | 2.555 | −34.681 | 1.00 | 44.45 |
| ATOM | 3652 | N | SER | A | 482 | −32.100 | 3.241 | −36.011 | 1.00 | 44.83 |
| ATOM | 3653 | CA | SER | A | 482 | −31.281 | 3.485 | −37.180 | 1.00 | 44.52 |
| ATOM | 3654 | CB | SER | A | 482 | −31.502 | 2.375 | −38.211 | 1.00 | 44.74 |
| ATOM | 3655 | OG | SER | A | 482 | −30.769 | 2.622 | −39.399 | 1.00 | 45.89 |
| ATOM | 3656 | C | SER | A | 482 | −31.661 | 4.836 | −37.765 | 1.00 | 43.84 |
| ATOM | 3657 | O | SER | A | 482 | −32.836 | 5.219 | −37.741 | 1.00 | 44.07 |
| ATOM | 3658 | N | GLY | A | 483 | −30.667 | 5.550 | −38.282 | 1.00 | 42.68 |
| ATOM | 3659 | CA | GLY | A | 483 | −30.872 | 6.872 | −38.853 | 1.00 | 41.64 |
| ATOM | 3660 | C | GLY | A | 483 | −30.085 | 7.095 | −40.130 | 1.00 | 41.05 |
| ATOM | 3661 | O | GLY | A | 483 | −29.430 | 6.179 | −40.647 | 1.00 | 41.09 |
| ATOM | 3662 | N | THR | A | 484 | −30.155 | 8.317 | −40.647 | 1.00 | 40.22 |
| ATOM | 3663 | CA | THR | A | 484 | −29.461 | 8.677 | −41.888 | 1.00 | 39.91 |
| ATOM | 3664 | CB | THR | A | 484 | −30.148 | 9.876 | −42.619 | 1.00 | 40.21 |
| ATOM | 3665 | OG1 | THR | A | 484 | −30.115 | 11.040 | −41.780 | 1.00 | 41.48 |
| ATOM | 3666 | CG2 | THR | A | 484 | −31.604 | 9.541 | −43.000 | 1.00 | 40.86 |
| ATOM | 3667 | C | THR | A | 484 | −27.995 | 9.033 | −41.603 | 1.00 | 38.43 |
| ATOM | 3668 | O | THR | A | 484 | −27.669 | 9.421 | −40.483 | 1.00 | 38.52 |
| ATOM | 3669 | N | PRO | A | 485 | −27.109 | 8.893 | −42.612 | 1.00 | 37.36 |
| ATOM | 3670 | CA | PRO | A | 485 | −25.695 | 9.226 | −42.413 | 1.00 | 36.29 |
| ATOM | 3671 | CB | PRO | A | 485 | −25.077 | 8.974 | −43.795 | 1.00 | 36.75 |
| ATOM | 3672 | CG | PRO | A | 485 | −25.997 | 7.985 | −44.442 | 1.00 | 37.04 |
| ATOM | 3673 | CD | PRO | A | 485 | −27.359 | 8.393 | −43.976 | 1.00 | 37.30 |
| ATOM | 3674 | C | PRO | A | 485 | −25.460 | 10.684 | −41.988 | 1.00 | 35.20 |
| ATOM | 3675 | O | PRO | A | 485 | −26.201 | 11.599 | −42.396 | 1.00 | 34.39 |
| ATOM | 3676 | N | TYR | A | 486 | −24.428 | 10.887 | −41.174 | 1.00 | 33.60 |
| ATOM | 3677 | CA | TYR | A | 486 | −24.025 | 12.233 | −40.782 | 1.00 | 32.74 |
| ATOM | 3678 | CB | TYR | A | 486 | −22.821 | 12.180 | −39.826 | 1.00 | 32.31 |
| ATOM | 3679 | CG | TYR | A | 486 | −22.348 | 13.564 | −39.452 | 1.00 | 32.79 |
| ATOM | 3680 | CD1 | TYR | A | 486 | −21.243 | 14.146 | −40.083 | 1.00 | 31.74 |
| ATOM | 3681 | CE1 | TYR | A | 486 | −20.827 | 15.430 | −39.742 | 1.00 | 30.85 |
| ATOM | 3682 | CZ | TYR | A | 486 | −21.527 | 16.141 | −38.778 | 1.00 | 31.99 |
| ATOM | 3683 | OH | TYR | A | 486 | −21.143 | 17.423 | −38.427 | 1.00 | 32.20 |
| ATOM | 3684 | CE2 | TYR | A | 486 | −22.629 | 15.588 | −38.160 | 1.00 | 31.88 |
| ATOM | 3685 | CD2 | TYR | A | 486 | −23.036 | 14.311 | −38.500 | 1.00 | 32.49 |
| ATOM | 3686 | C | TYR | A | 486 | −23.652 | 13.082 | −41.999 | 1.00 | 31.99 |
| ATOM | 3687 | O | TYR | A | 486 | −22.949 | 12.602 | −42.900 | 1.00 | 31.62 |
| ATOM | 3688 | N | THR | A | 487 | −24.106 | 14.336 | −42.004 | 1.00 | 31.14 |
| ATOM | 3689 | CA | THR | A | 487 | −23.676 | 15.336 | −42.986 | 1.00 | 31.43 |
| ATOM | 3690 | CB | THR | A | 487 | −24.879 | 15.785 | −43.869 | 1.00 | 31.94 |
| ATOM | 3691 | OG1 | THR | A | 487 | −25.321 | 14.665 | −44.644 | 1.00 | 35.19 |
| ATOM | 3692 | CG2 | THR | A | 487 | −24.489 | 16.904 | −44.810 | 1.00 | 32.95 |
| ATOM | 3693 | C | THR | A | 487 | −23.110 | 16.561 | −42.261 | 1.00 | 29.79 |
| ATOM | 3694 | O | THR | A | 487 | −23.761 | 17.078 | −41.363 | 1.00 | 29.20 |
| ATOM | 3695 | N | PRO | A | 488 | −21.901 | 17.027 | −42.644 | 1.00 | 29.20 |
| ATOM | 3696 | CA | PRO | A | 488 | −21.309 | 18.228 | −42.005 | 1.00 | 28.77 |
| ATOM | 3697 | CB | PRO | A | 488 | −19.988 | 18.435 | −42.763 | 1.00 | 28.75 |
| ATOM | 3698 | CG | PRO | A | 488 | −19.684 | 17.126 | −43.408 | 1.00 | 29.52 |
| ATOM | 3699 | CD | PRO | A | 488 | −21.010 | 16.448 | −43.667 | 1.00 | 29.41 |
| ATOM | 3700 | C | PRO | A | 488 | −22.175 | 19.463 | −42.194 | 1.00 | 28.44 |
| ATOM | 3701 | O | PRO | A | 488 | −23.003 | 19.499 | −43.116 | 1.00 | 28.51 |
| ATOM | 3702 | N | LEU | A | 489 | −21.971 | 20.469 | −41.345 | 1.00 | 27.28 |
| ATOM | 3703 | CA | LEU | A | 489 | −22.606 | 21.775 | −41.522 | 1.00 | 26.52 |
| ATOM | 3704 | CB | LEU | A | 489 | −22.269 | 22.708 | −40.365 | 1.00 | 27.19 |
| ATOM | 3705 | CG | LEU | A | 489 | −22.805 | 22.303 | −38.987 | 1.00 | 27.53 |
| ATOM | 3706 | CD1 | LEU | A | 489 | −22.233 | 23.242 | −37.929 | 1.00 | 26.81 |
| ATOM | 3707 | CD2 | LEU | A | 489 | −24.332 | 22.323 | −38.970 | 1.00 | 28.98 |
| ATOM | 3708 | C | LEU | A | 489 | −22.137 | 22.402 | −42.833 | 1.00 | 26.65 |
| ATOM | 3709 | O | LEU | A | 489 | −20.983 | 22.210 | −43.245 | 1.00 | 25.40 |
| ATOM | 3710 | N | PRO | A | 490 | −23.030 | 23.153 | −43.503 | 1.00 | 26.84 |
| ATOM | 3711 | CA | PRO | A | 490 | −22.636 | 23.745 | −44.786 | 1.00 | 26.44 |
| ATOM | 3712 | CB | PRO | A | 490 | −23.983 | 24.107 | −45.432 | 1.00 | 27.00 |
| ATOM | 3713 | CG | PRO | A | 490 | −24.900 | 24.341 | −44.289 | 1.00 | 27.68 |
| ATOM | 3714 | CD | PRO | A | 490 | −24.425 | 23.475 | −43.137 | 1.00 | 26.91 |
| ATOM | 3715 | C | PRO | A | 490 | −21.737 | 24.982 | −44.668 | 1.00 | 26.22 |
| ATOM | 3716 | O | PRO | A | 490 | −21.826 | 25.729 | −43.698 | 1.00 | 25.74 |
| ATOM | 3717 | N | CYS | A | 491 | −20.858 | 25.182 | −45.650 | 1.00 | 26.06 |
| ATOM | 3718 | CA | CYS | A | 491 | −20.079 | 26.412 | −45.754 | 1.00 | 26.88 |
| ATOM | 3719 | CB | CYS | A | 491 | −18.630 | 26.194 | −45.302 | 1.00 | 27.00 |
| ATOM | 3720 | SG | CYS | A | 491 | −18.450 | 25.196 | −43.819 | 1.00 | 27.23 |
| ATOM | 3721 | C | CYS | A | 491 | −20.032 | 26.822 | −47.217 | 1.00 | 27.27 |
| ATOM | 3722 | O | CYS | A | 491 | −20.369 | 26.026 | −48.083 | 1.00 | 27.34 |
| ATOM | 3723 | N | ALA | A | 492 | −19.577 | 28.045 | −47.484 | 1.00 | 28.05 |
| ATOM | 3724 | CA | ALA | A | 492 | −19.205 | 28.449 | −48.845 | 1.00 | 29.40 |
| ATOM | 3725 | CB | ALA | A | 492 | −18.837 | 29.928 | −48.866 | 1.00 | 29.49 |
| ATOM | 3726 | C | ALA | A | 492 | −18.023 | 27.599 | −49.320 | 1.00 | 30.37 |
| ATOM | 3727 | O | ALA | A | 492 | −17.310 | 26.998 | −48.497 | 1.00 | 30.36 |
| ATOM | 3728 | N | THR | A | 493 | −17.828 | 27.508 | −50.633 | 1.00 | 31.27 |
| ATOM | 3729 | CA | THR | A | 493 | −16.612 | 26.883 | −51.163 | 1.00 | 32.43 |
| ATOM | 3730 | CB | THR | A | 493 | −16.845 | 26.234 | −52.533 | 1.00 | 33.22 |
| ATOM | 3731 | OG1 | THR | A | 493 | −17.944 | 25.324 | −52.431 | 1.00 | 38.55 |
| ATOM | 3732 | CG2 | THR | A | 493 | −15.590 | 25.464 | −52.996 | 1.00 | 33.71 |
| ATOM | 3733 | C | THR | A | 493 | −15.596 | 28.006 | −51.254 | 1.00 | 31.35 |
| ATOM | 3734 | O | THR | A | 493 | −15.916 | 29.068 | −51.795 | 1.00 | 31.58 |
| ATOM | 3735 | N | PRO | A | 494 | −14.390 | 27.815 | −50.682 | 1.00 | 30.44 |
| ATOM | 3736 | CA | PRO | A | 494 | −13.464 | 28.947 | −50.696 | 1.00 | 30.01 |
| ATOM | 3737 | CB | PRO | A | 494 | −12.414 | 28.555 | −49.645 | 1.00 | 30.26 |
| ATOM | 3738 | CG | PRO | A | 494 | −12.416 | 27.077 | −49.658 | 1.00 | 30.56 |
| ATOM | 3739 | CD | PRO | A | 494 | −13.815 | 26.635 | −49.997 | 1.00 | 30.85 |
| ATOM | 3740 | C | PRO | A | 494 | −12.809 | 29.089 | −52.060 | 1.00 | 28.81 |
| ATOM | 3741 | O | PRO | A | 494 | −12.801 | 28.137 | −52.834 | 1.00 | 28.82 |
| ATOM | 3742 | N | THR | A | 495 | −12.260 | 30.258 | −52.352 | 1.00 | 28.43 |
| ATOM | 3743 | CA | THR | A | 495 | −11.551 | 30.419 | −53.623 | 1.00 | 28.44 |
| ATOM | 3744 | CB | THR | A | 495 | −11.885 | 31.748 | −54.319 | 1.00 | 28.68 |
| ATOM | 3745 | OG1 | THR | A | 495 | −11.449 | 32.839 | −53.500 | 1.00 | 30.39 |
| ATOM | 3746 | CG2 | THR | A | 495 | −13.383 | 31.858 | −54.564 | 1.00 | 29.85 |
| ATOM | 3747 | C | THR | A | 495 | −10.057 | 30.335 | −53.404 | 1.00 | 27.94 |
| ATOM | 3748 | O | THR | A | 495 | −9.289 | 30.159 | −54.352 | 1.00 | 27.63 |
| ATOM | 3749 | N | SER | A | 496 | −9.671 | 30.463 | −52.139 | 1.00 | 27.51 |
| ATOM | 3750 | CA | SER | A | 496 | −8.279 | 30.492 | −51.722 | 1.00 | 27.18 |
| ATOM | 3751 | CB | SER | A | 496 | −7.928 | 31.916 | −51.329 | 1.00 | 27.72 |
| ATOM | 3752 | OG | SER | A | 496 | −6.531 | 32.076 | −51.240 | 1.00 | 32.60 |
| ATOM | 3753 | C | SER | A | 496 | −8.134 | 29.583 | −50.501 | 1.00 | 25.85 |
| ATOM | 3754 | O | SER | A | 496 | −9.024 | 29.548 | −49.634 | 1.00 | 25.10 |
| ATOM | 3755 | N | VAL | A | 497 | −7.039 | 28.824 | −50.430 | 1.00 | 24.43 |
| ATOM | 3756 | CA | VAL | A | 497 | −6.801 | 28.032 | −49.209 | 1.00 | 22.17 |
| ATOM | 3757 | CB | VAL | A | 497 | −7.281 | 26.511 | −49.290 | 1.00 | 23.00 |
| ATOM | 3758 | CG1 | VAL | A | 497 | −6.224 | 25.445 | −48.881 | 1.00 | 22.48 |
| ATOM | 3759 | CG2 | VAL | A | 497 | −8.049 | 26.161 | −50.578 | 1.00 | 22.62 |
| ATOM | 3760 | C | VAL | A | 497 | −5.388 | 28.251 | −48.672 | 1.00 | 21.13 |
| ATOM | 3761 | O | VAL | A | 497 | −4.419 | 28.359 | −49.439 | 1.00 | 20.38 |
| ATOM | 3762 | N | ALA | A | 498 | −5.302 | 28.395 | −47.355 | 1.00 | 19.70 |
| ATOM | 3763 | CA | ALA | A | 498 | −4.020 | 28.576 | −46.702 | 1.00 | 18.92 |
| ATOM | 3764 | CB | ALA | A | 498 | −4.226 | 29.126 | −45.290 | 1.00 | 19.31 |
| ATOM | 3765 | C | ALA | A | 498 | −3.396 | 27.185 | −46.655 | 1.00 | 18.67 |
| ATOM | 3766 | O | ALA | A | 498 | −3.966 | 26.266 | −46.047 | 1.00 | 19.29 |
| ATOM | 3767 | N | VAL | A | 499 | −2.252 | 27.021 | −47.319 | 1.00 | 17.23 |
| ATOM | 3768 | CA | VAL | A | 499 | −1.551 | 25.735 | −47.361 | 1.00 | 16.09 |
| ATOM | 3769 | CB | VAL | A | 499 | −1.165 | 25.347 | −48.814 | 1.00 | 16.97 |
| ATOM | 3770 | CG1 | VAL | A | 499 | −0.403 | 23.984 | −48.863 | 1.00 | 16.08 |
| ATOM | 3771 | CG2 | VAL | A | 499 | −2.413 | 25.291 | −49.696 | 1.00 | 17.03 |
| ATOM | 3772 | C | VAL | A | 499 | −0.306 | 25.841 | −46.491 | 1.00 | 15.95 |
| ATOM | 3773 | O | VAL | A | 499 | 0.604 | 26.607 | −46.791 | 1.00 | 16.15 |
| ATOM | 3774 | N | THR | A | 500 | −0.279 | 25.085 | −45.404 | 1.00 | 15.16 |
| ATOM | 3775 | CA | THR | A | 500 | 0.863 | 25.116 | −44.505 | 1.00 | 14.85 |
| ATOM | 3776 | CB | THR | A | 500 | 0.415 | 24.916 | −43.035 | 1.00 | 14.76 |
| ATOM | 3777 | OG1 | THR | A | 500 | −0.403 | 26.022 | −42.635 | 1.00 | 16.00 |
| ATOM | 3778 | CG2 | THR | A | 500 | 1.639 | 24.856 | −42.136 | 1.00 | 15.39 |
| ATOM | 3779 | C | THR | A | 500 | 1.796 | 23.993 | −44.932 | 1.00 | 14.99 |
| ATOM | 3780 | O | THR | A | 500 | 1.480 | 22.804 | −44.792 | 1.00 | 14.81 |
| ATOM | 3781 | N | PHE | A | 501 | 2.941 | 24.370 | −45.481 | 1.00 | 14.71 |
| ATOM | 3782 | CA | PHE | A | 501 | 3.981 | 23.411 | −45.793 | 1.00 | 15.64 |
| ATOM | 3783 | CB | PHE | A | 501 | 4.943 | 23.964 | −46.832 | 1.00 | 15.86 |
| ATOM | 3784 | CG | PHE | A | 501 | 4.289 | 24.172 | −48.168 | 1.00 | 18.38 |
| ATOM | 3785 | CD1 | PHE | A | 501 | 3.676 | 25.388 | −48.469 | 1.00 | 19.85 |
| ATOM | 3786 | CE1 | PHE | A | 501 | 3.052 | 25.581 | −49.709 | 1.00 | 21.58 |
| ATOM | 3787 | CZ | PHE | A | 501 | 3.015 | 24.547 | −50.642 | 1.00 | 19.80 |
| ATOM | 3788 | CE2 | PHE | A | 501 | 3.607 | 23.324 | −50.356 | 1.00 | 21.69 |
| ATOM | 3789 | CD2 | PHE | A | 501 | 4.231 | 23.134 | −49.095 | 1.00 | 21.96 |
| ATOM | 3790 | C | PHE | A | 501 | 4.711 | 23.009 | −44.536 | 1.00 | 15.81 |
| ATOM | 3791 | O | PHE | A | 501 | 5.207 | 23.852 | −43.804 | 1.00 | 16.78 |
| ATOM | 3792 | N | HIS | A | 502 | 4.789 | 21.698 | −44.317 | 1.00 | 14.85 |
| ATOM | 3793 | CA | HIS | A | 502 | 5.239 | 21.175 | −43.027 | 1.00 | 14.17 |
| ATOM | 3794 | CB | HIS | A | 502 | 3.987 | 20.565 | −42.356 | 1.00 | 14.85 |
| ATOM | 3795 | CG | HIS | A | 502 | 4.221 | 19.875 | −41.054 | 1.00 | 13.69 |
| ATOM | 3796 | ND1 | HIS | A | 502 | 4.819 | 18.637 | −40.966 | 1.00 | 12.55 |
| ATOM | 3797 | CE1 | HIS | A | 502 | 4.816 | 18.241 | −39.702 | 1.00 | 15.76 |
| ATOM | 3798 | NE2 | HIS | A | 502 | 4.191 | 19.155 | −38.980 | 1.00 | 14.42 |
| ATOM | 3799 | CD2 | HIS | A | 502 | 3.797 | 20.183 | −39.804 | 1.00 | 14.92 |
| ATOM | 3800 | C | HIS | A | 502 | 6.317 | 20.161 | −43.412 | 1.00 | 14.16 |
| ATOM | 3801 | O | HIS | A | 502 | 6.013 | 19.043 | −43.824 | 1.00 | 13.85 |
| ATOM | 3802 | N | GLU | A | 503 | 7.577 | 20.590 | −43.352 | 1.00 | 13.63 |
| ATOM | 3803 | CA | GLU | A | 503 | 8.678 | 19.821 | −43.968 | 1.00 | 14.46 |
| ATOM | 3804 | CB | GLU | A | 503 | 9.434 | 20.712 | −44.996 | 1.00 | 14.22 |
| ATOM | 3805 | CG | GLU | A | 503 | 10.782 | 20.121 | −45.524 | 1.00 | 16.31 |
| ATOM | 3806 | CD | GLU | A | 503 | 10.620 | 18.973 | −46.539 | 1.00 | 21.32 |
| ATOM | 3807 | OE1 | GLU | A | 503 | 11.523 | 18.819 | −47.393 | 1.00 | 21.21 |
| ATOM | 3808 | OE2 | GLU | A | 503 | 9.609 | 18.230 | −46.510 | 1.00 | 20.10 |
| ATOM | 3809 | C | GLU | A | 503 | 9.657 | 19.322 | −42.917 | 1.00 | 14.22 |
| ATOM | 3810 | O | GLU | A | 503 | 10.175 | 20.121 | −42.131 | 1.00 | 15.31 |
| ATOM | 3811 | N | LEU | A | 504 | 9.960 | 18.027 | −42.927 | 1.00 | 14.92 |
| ATOM | 3812 | CA | LEU | A | 504 | 11.026 | 17.518 | −42.052 | 1.00 | 15.49 |
| ATOM | 3813 | CB | LEU | A | 504 | 10.658 | 16.147 | −41.489 | 1.00 | 16.33 |
| ATOM | 3814 | CG | LEU | A | 504 | 9.479 | 16.178 | −40.498 | 1.00 | 17.19 |
| ATOM | 3815 | CD1 | LEU | A | 504 | 8.922 | 14.753 | −40.320 | 1.00 | 19.08 |
| ATOM | 3816 | CD2 | LEU | A | 504 | 9.953 | 16.723 | −39.198 | 1.00 | 17.28 |
| ATOM | 3817 | C | LEU | A | 504 | 12.318 | 17.428 | −42.846 | 1.00 | 16.59 |
| ATOM | 3818 | O | LEU | A | 504 | 12.403 | 16.656 | −43.785 | 1.00 | 16.72 |
| ATOM | 3819 | N | VAL | A | 505 | 13.317 | 18.201 | −42.444 | 1.00 | 17.20 |
| ATOM | 3820 | CA | VAL | A | 505 | 14.592 | 18.235 | −43.154 | 1.00 | 19.16 |
| ATOM | 3821 | CB | VAL | A | 505 | 14.548 | 19.141 | −44.418 | 1.00 | 18.88 |
| ATOM | 3822 | CG1 | VAL | A | 505 | 14.028 | 20.539 | −44.090 | 1.00 | 19.28 |
| ATOM | 3823 | CG2 | VAL | A | 505 | 15.948 | 19.219 | −45.095 | 1.00 | 21.65 |
| ATOM | 3824 | C | VAL | A | 505 | 15.674 | 18.705 | −42.188 | 1.00 | 20.00 |
| ATOM | 3825 | O | VAL | A | 505 | 15.595 | 19.785 | −41.595 | 1.00 | 19.92 |
| ATOM | 3826 | N | SER | A | 506 | 16.685 | 17.868 | −42.011 | 1.00 | 21.73 |
| ATOM | 3827 | CA | SER | A | 506 | 17.761 | 18.216 | −41.104 | 1.00 | 23.33 |
| ATOM | 3828 | CB | SER | A | 506 | 18.570 | 16.974 | −40.771 | 1.00 | 23.74 |
| ATOM | 3829 | OG | SER | A | 506 | 19.583 | 17.320 | −39.847 | 1.00 | 28.30 |
| ATOM | 3830 | C | SER | A | 506 | 18.646 | 19.284 | −41.759 | 1.00 | 23.03 |
| ATOM | 3831 | O | SER | A | 506 | 19.070 | 19.139 | −42.908 | 1.00 | 23.20 |
| ATOM | 3832 | N | THR | A | 507 | 18.888 | 20.371 | −41.049 | 1.00 | 24.01 |
| ATOM | 3833 | CA | THR | A | 507 | 19.685 | 21.464 | −41.600 | 1.00 | 25.15 |
| ATOM | 3834 | CB | THR | A | 507 | 18.845 | 22.725 | −41.833 | 1.00 | 25.01 |
| ATOM | 3835 | OG1 | THR | A | 507 | 18.104 | 23.015 | −40.650 | 1.00 | 24.43 |
| ATOM | 3836 | CG2 | THR | A | 507 | 17.891 | 22.536 | −43.000 | 1.00 | 24.71 |
| ATOM | 3837 | C | THR | A | 507 | 20.795 | 21.812 | −40.623 | 1.00 | 27.09 |
| ATOM | 3838 | O | THR | A | 507 | 20.729 | 21.448 | −39.451 | 1.00 | 26.15 |
| ATOM | 3839 | N | GLN | A | 508 | 21.798 | 22.536 | −41.113 | 1.00 | 29.34 |
| ATOM | 3840 | CA | GLN | A | 508 | 22.912 | 22.986 | −40.272 | 1.00 | 32.54 |
| ATOM | 3841 | CB | GLN | A | 508 | 24.239 | 22.542 | −40.897 | 1.00 | 32.54 |
| ATOM | 3842 | CG | GLN | A | 508 | 24.369 | 21.010 | −40.972 | 1.00 | 34.32 |
| ATOM | 3843 | CD | GLN | A | 508 | 25.400 | 20.515 | −41.991 | 1.00 | 36.23 |
| ATOM | 3844 | OE1 | GLN | A | 508 | 26.283 | 19.700 | −41.660 | 1.00 | 41.79 |
| ATOM | 3845 | NE2 | GLN | A | 508 | 25.279 | 20.977 | −43.242 | 1.00 | 40.42 |
| ATOM | 3846 | C | GLN | A | 508 | 22.827 | 24.502 | −40.100 | 1.00 | 33.06 |
| ATOM | 3847 | O | GLN | A | 508 | 22.136 | 25.178 | −40.873 | 1.00 | 32.49 |
| ATOM | 3848 | N | PHE | A | 509 | 23.494 | 25.037 | −39.075 | 1.00 | 33.80 |
| ATOM | 3849 | CA | PHE | A | 509 | 23.432 | 26.476 | −38.782 | 1.00 | 35.03 |
| ATOM | 3850 | CB | PHE | A | 509 | 24.413 | 26.837 | −37.651 | 1.00 | 36.75 |
| ATOM | 3851 | CG | PHE | A | 509 | 24.481 | 28.315 | −37.340 | 1.00 | 39.07 |
| ATOM | 3852 | CD1 | PHE | A | 509 | 23.592 | 28.893 | −36.428 | 1.00 | 41.58 |
| ATOM | 3853 | CE1 | PHE | A | 509 | 23.642 | 30.265 | −36.140 | 1.00 | 42.61 |
| ATOM | 3854 | CZ | PHE | A | 509 | 24.603 | 31.073 | −36.766 | 1.00 | 41.78 |
| ATOM | 3855 | CE2 | PHE | A | 509 | 25.507 | 30.503 | −37.678 | 1.00 | 42.48 |
| ATOM | 3856 | CD2 | PHE | A | 509 | 25.441 | 29.127 | −37.955 | 1.00 | 41.46 |
| ATOM | 3857 | C | PHE | A | 509 | 23.712 | 27.311 | −40.040 | 1.00 | 34.58 |
| ATOM | 3858 | O | PHE | A | 509 | 24.614 | 26.990 | −40.815 | 1.00 | 34.58 |
| ATOM | 3859 | N | GLY | A | 510 | 22.912 | 28.355 | −40.256 | 1.00 | 33.85 |
| ATOM | 3860 | CA | GLY | A | 510 | 23.101 | 29.241 | −41.407 | 1.00 | 33.22 |
| ATOM | 3861 | C | GLY | A | 510 | 22.352 | 28.826 | −42.671 | 1.00 | 32.36 |
| ATOM | 3862 | O | GLY | A | 510 | 22.369 | 29.545 | −43.679 | 1.00 | 32.97 |
| ATOM | 3863 | N | GLN | A | 511 | 21.705 | 27.663 | −42.628 | 1.00 | 30.50 |
| ATOM | 3864 | CA | GLN | A | 511 | 20.885 | 27.217 | −43.745 | 1.00 | 29.02 |
| ATOM | 3865 | CB | GLN | A | 511 | 21.026 | 25.712 | −43.931 | 1.00 | 28.92 |
| ATOM | 3866 | CG | GLN | A | 511 | 22.436 | 25.276 | −44.349 | 1.00 | 29.91 |
| ATOM | 3867 | CD | GLN | A | 511 | 22.571 | 23.776 | −44.439 | 1.00 | 31.36 |
| ATOM | 3868 | OE1 | GLN | A | 511 | 21.760 | 23.036 | −43.879 | 1.00 | 31.69 |
| ATOM | 3869 | NE2 | GLN | A | 511 | 23.590 | 23.309 | −45.160 | 1.00 | 30.72 |
| ATOM | 3870 | C | GLN | A | 511 | 19.418 | 27.619 | −43.543 | 1.00 | 27.82 |
| ATOM | 3871 | O | GLN | A | 511 | 18.928 | 27.695 | −42.399 | 1.00 | 27.36 |
| ATOM | 3872 | N | THR | A | 512 | 18.727 | 27.895 | −44.650 | 1.00 | 25.92 |
| ATOM | 3873 | CA | THR | A | 512 | 17.305 | 28.271 | −44.613 | 1.00 | 24.51 |
| ATOM | 3874 | CB | THR | A | 512 | 17.126 | 29.763 | −44.994 | 1.00 | 24.95 |
| ATOM | 3875 | OG1 | THR | A | 512 | 17.769 | 30.580 | −44.004 | 1.00 | 27.43 |
| ATOM | 3876 | CG2 | THR | A | 512 | 15.653 | 30.151 | −45.069 | 1.00 | 25.94 |
| ATOM | 3877 | C | THR | A | 512 | 16.536 | 27.384 | −45.600 | 1.00 | 23.09 |
| ATOM | 3878 | O | THR | A | 512 | 16.994 | 27.152 | −46.717 | 1.00 | 22.75 |
| ATOM | 3879 | N | VAL | A | 513 | 15.376 | 26.877 | −45.200 | 1.00 | 20.62 |
| ATOM | 3880 | CA | VAL | A | 513 | 14.593 | 26.074 | −46.136 | 1.00 | 19.05 |
| ATOM | 3881 | CB | VAL | A | 513 | 13.946 | 24.855 | −45.428 | 1.00 | 19.18 |
| ATOM | 3882 | CG1 | VAL | A | 513 | 13.041 | 24.064 | −46.397 | 1.00 | 18.87 |
| ATOM | 3883 | CG2 | VAL | A | 513 | 15.042 | 23.938 | −44.895 | 1.00 | 20.56 |
| ATOM | 3884 | C | VAL | A | 513 | 13.536 | 26.979 | −46.748 | 1.00 | 17.98 |
| ATOM | 3885 | O | VAL | A | 513 | 12.910 | 27.768 | −46.029 | 1.00 | 16.65 |
| ATOM | 3886 | N | LYS | A | 514 | 13.346 | 26.857 | −48.063 | 1.00 | 17.91 |
| ATOM | 3887 | CA | LYS | A | 514 | 12.279 | 27.583 | −48.757 | 1.00 | 18.14 |
| ATOM | 3888 | CB | LYS | A | 514 | 12.845 | 28.712 | −49.638 | 1.00 | 17.66 |
| ATOM | 3889 | CG | LYS | A | 514 | 13.867 | 29.576 | −48.945 | 1.00 | 19.26 |
| ATOM | 3890 | CD | LYS | A | 514 | 14.197 | 30.839 | −49.765 | 1.00 | 21.27 |
| ATOM | 3891 | CE | LYS | A | 514 | 15.224 | 31.675 | −49.001 | 1.00 | 26.06 |
| ATOM | 3892 | NZ | LYS | A | 514 | 15.461 | 33.022 | −49.626 | 1.00 | 28.70 |
| ATOM | 3893 | C | LYS | A | 514 | 11.494 | 26.621 | −49.625 | 1.00 | 18.22 |
| ATOM | 3894 | O | LYS | A | 514 | 11.949 | 25.502 | −49.912 | 1.00 | 18.39 |
| ATOM | 3895 | N | VAL | A | 515 | 10.304 | 27.037 | −50.045 | 1.00 | 18.20 |
| ATOM | 3896 | CA | VAL | A | 515 | 9.546 | 26.212 | −50.980 | 1.00 | 19.00 |
| ATOM | 3897 | CB | VAL | A | 515 | 8.198 | 25.731 | −50.404 | 1.00 | 20.00 |
| ATOM | 3898 | CG1 | VAL | A | 515 | 7.403 | 26.904 | −49.879 | 1.00 | 21.01 |
| ATOM | 3899 | CG2 | VAL | A | 515 | 7.417 | 24.903 | −51.447 | 1.00 | 20.10 |
| ATOM | 3900 | C | VAL | A | 515 | 9.421 | 26.973 | −52.302 | 1.00 | 18.89 |
| ATOM | 3901 | O | VAL | A | 515 | 9.079 | 28.159 | −52.317 | 1.00 | 18.67 |
| ATOM | 3902 | N | ALA | A | 516 | 9.781 | 26.295 | −53.390 | 1.00 | 19.87 |
| ATOM | 3903 | CA | ALA | A | 516 | 9.796 | 26.898 | −54.732 | 1.00 | 20.38 |
| ATOM | 3904 | CB | ALA | A | 516 | 11.177 | 26.768 | −55.356 | 1.00 | 20.58 |
| ATOM | 3905 | C | ALA | A | 516 | 8.789 | 26.110 | −55.525 | 1.00 | 20.65 |
| ATOM | 3906 | O | ALA | A | 516 | 8.638 | 24.910 | −55.303 | 1.00 | 20.40 |
| ATOM | 3907 | N | GLY | A | 517 | 8.075 | 26.765 | −56.430 | 1.00 | 20.80 |
| ATOM | 3908 | CA | GLY | A | 517 | 7.092 | 26.039 | −57.214 | 1.00 | 22.32 |
| ATOM | 3909 | C | GLY | A | 517 | 6.536 | 26.853 | −58.352 | 1.00 | 22.86 |
| ATOM | 3910 | O | GLY | A | 517 | 6.902 | 28.024 | −58.527 | 1.00 | 22.58 |
| ATOM | 3911 | N | ASN | A | 518 | 5.642 | 26.233 | −59.116 | 1.00 | 24.66 |
| ATOM | 3912 | CA | ASN | A | 518 | 5.201 | 26.817 | −60.390 | 1.00 | 26.74 |
| ATOM | 3913 | CB | ASN | A | 518 | 4.670 | 25.754 | −61.354 | 1.00 | 26.97 |
| ATOM | 3914 | CG | ASN | A | 518 | 3.386 | 25.117 | −60.872 | 1.00 | 31.69 |
| ATOM | 3915 | OD1 | ASN | A | 518 | 3.004 | 25.226 | −59.677 | 1.00 | 28.89 |
| ATOM | 3916 | ND2 | ASN | A | 518 | 2.707 | 24.419 | −61.786 | 1.00 | 31.65 |
| ATOM | 3917 | C | ASN | A | 518 | 4.199 | 27.937 | −60.232 | 1.00 | 27.20 |
| ATOM | 3918 | O | ASN | A | 518 | 4.154 | 28.822 | −61.079 | 1.00 | 28.68 |
| ATOM | 3919 | N | ALA | A | 519 | 3.399 | 27.907 | −59.163 | 1.00 | 27.04 |
| ATOM | 3920 | CA | ALA | A | 519 | 2.424 | 28.978 | −58.898 | 1.00 | 26.47 |
| ATOM | 3921 | CB | ALA | A | 519 | 1.473 | 28.598 | −57.747 | 1.00 | 27.02 |
| ATOM | 3922 | C | ALA | A | 519 | 3.090 | 30.322 | −58.629 | 1.00 | 26.35 |
| ATOM | 3923 | O | ALA | A | 519 | 4.226 | 30.394 | −58.135 | 1.00 | 25.27 |
| ATOM | 3924 | N | ALA | A | 520 | 2.369 | 31.394 | −58.954 | 1.00 | 26.25 |
| ATOM | 3925 | CA | ALA | A | 520 | 2.887 | 32.741 | −58.784 | 1.00 | 26.77 |
| ATOM | 3926 | CB | ALA | A | 520 | 1.872 | 33.775 | −59.298 | 1.00 | 27.50 |
| ATOM | 3927 | C | ALA | A | 520 | 3.250 | 33.004 | −57.317 | 1.00 | 26.68 |
| ATOM | 3928 | O | ALA | A | 520 | 4.301 | 33.560 | −57.030 | 1.00 | 26.01 |
| ATOM | 3929 | N | ALA | A | 521 | 2.395 | 32.548 | −56.399 | 1.00 | 26.75 |
| ATOM | 3930 | CA | ALA | A | 521 | 2.628 | 32.712 | −54.963 | 1.00 | 26.82 |
| ATOM | 3931 | CB | ALA | A | 521 | 1.395 | 32.251 | −54.167 | 1.00 | 26.85 |
| ATOM | 3932 | C | ALA | A | 521 | 3.876 | 31.950 | −54.504 | 1.00 | 26.51 |
| ATOM | 3933 | O | ALA | A | 521 | 4.485 | 32.305 | −53.494 | 1.00 | 26.63 |
| ATOM | 3934 | N | LEU | A | 522 | 4.261 | 30.919 | −55.259 | 1.00 | 26.79 |
| ATOM | 3935 | CA | LEU | A | 522 | 5.452 | 30.113 | −54.932 | 1.00 | 26.50 |
| ATOM | 3936 | CB | LEU | A | 522 | 5.185 | 28.626 | −55.155 | 1.00 | 26.64 |
| ATOM | 3937 | CG | LEU | A | 522 | 4.224 | 27.946 | −54.169 | 1.00 | 26.58 |
| ATOM | 3938 | CD1 | LEU | A | 522 | 4.049 | 26.489 | −54.533 | 1.00 | 27.59 |
| ATOM | 3939 | CD2 | LEU | A | 522 | 4.718 | 28.092 | −52.730 | 1.00 | 28.08 |
| ATOM | 3940 | C | LEU | A | 522 | 6.696 | 30.559 | −55.709 | 1.00 | 26.54 |
| ATOM | 3941 | O | LEU | A | 522 | 7.779 | 29.987 | −55.547 | 1.00 | 25.56 |
| ATOM | 3942 | N | GLY | A | 523 | 6.518 | 31.575 | −56.552 | 1.00 | 26.25 |
| ATOM | 3943 | CA | GLY | A | 523 | 7.637 | 32.267 | −57.199 | 1.00 | 26.16 |
| ATOM | 3944 | C | GLY | A | 523 | 7.996 | 31.809 | −58.607 | 1.00 | 26.84 |
| ATOM | 3945 | O | GLY | A | 523 | 9.029 | 32.227 | −59.152 | 1.00 | 25.81 |
| ATOM | 3946 | N | ASN | A | 524 | 7.162 | 30.946 | −59.193 | 1.00 | 27.13 |
| ATOM | 3947 | CA | ASN | A | 524 | 7.413 | 30.419 | −60.539 | 1.00 | 27.74 |
| ATOM | 3948 | CB | ASN | A | 524 | 7.046 | 31.484 | −61.591 | 1.00 | 28.43 |
| ATOM | 3949 | CG | ASN | A | 524 | 7.123 | 30.960 | −63.015 | 1.00 | 30.79 |
| ATOM | 3950 | OD1 | ASN | A | 524 | 6.856 | 29.780 | −63.285 | 1.00 | 30.80 |
| ATOM | 3951 | ND2 | ASN | A | 524 | 7.515 | 31.838 | −63.936 | 1.00 | 33.61 |
| ATOM | 3952 | C | ASN | A | 524 | 8.845 | 29.857 | −60.710 | 1.00 | 28.44 |
| ATOM | 3953 | O | ASN | A | 524 | 9.531 | 30.104 | −61.720 | 1.00 | 27.82 |
| ATOM | 3954 | N | TRP | A | 525 | 9.280 | 29.111 | −59.693 | 1.00 | 27.99 |
| ATOM | 3955 | CA | TRP | A | 525 | 10.573 | 28.398 | −59.659 | 1.00 | 28.92 |
| ATOM | 3956 | CB | TRP | A | 525 | 10.787 | 27.507 | −60.896 | 1.00 | 28.31 |
| ATOM | 3957 | CG | TRP | A | 525 | 9.803 | 26.394 | −61.060 | 1.00 | 27.68 |
| ATOM | 3958 | CD1 | TRP | A | 525 | 8.902 | 26.247 | −62.078 | 1.00 | 27.96 |
| ATOM | 3959 | NE1 | TRP | A | 525 | 8.166 | 25.106 | −61.907 | 1.00 | 27.55 |
| ATOM | 3960 | CE2 | TRP | A | 525 | 8.589 | 24.471 | −60.762 | 1.00 | 30.58 |
| ATOM | 3961 | CD2 | TRP | A | 525 | 9.609 | 25.277 | −60.184 | 1.00 | 27.84 |
| ATOM | 3962 | CE3 | TRP | A | 525 | 10.230 | 24.842 | −59.001 | 1.00 | 26.50 |
| ATOM | 3963 | CZ3 | TRP | A | 525 | 9.787 | 23.655 | −58.411 | 1.00 | 27.55 |
| ATOM | 3964 | CH2 | TRP | A | 525 | 8.752 | 22.889 | −58.998 | 1.00 | 27.90 |
| ATOM | 3965 | CZ2 | TRP | A | 525 | 8.144 | 23.279 | −60.168 | 1.00 | 26.22 |
| ATOM | 3966 | C | TRP | A | 525 | 11.790 | 29.301 | −59.452 | 1.00 | 29.66 |
| ATOM | 3967 | O | TRP | A | 525 | 12.921 | 28.804 | −59.346 | 1.00 | 30.61 |
| ATOM | 3968 | N | SER | A | 526 | 11.570 | 30.613 | −59.380 | 1.00 | 30.33 |
| ATOM | 3969 | CA | SER | A | 526 | 12.645 | 31.536 | −59.004 | 1.00 | 31.13 |
| ATOM | 3970 | CB | SER | A | 526 | 12.213 | 32.993 | −59.187 | 1.00 | 31.02 |
| ATOM | 3971 | OG | SER | A | 526 | 13.166 | 33.838 | −58.562 | 1.00 | 33.69 |
| ATOM | 3972 | C | SER | A | 526 | 13.086 | 31.312 | −57.560 | 1.00 | 31.29 |
| ATOM | 3973 | O | SER | A | 526 | 12.271 | 31.381 | −56.627 | 1.00 | 31.21 |
| ATOM | 3974 | N | THR | A | 527 | 14.373 | 31.049 | −57.367 | 1.00 | 31.34 |
| ATOM | 3975 | CA | THR | A | 527 | 14.880 | 30.794 | −56.021 | 1.00 | 31.64 |
| ATOM | 3976 | CB | THR | A | 527 | 16.259 | 30.098 | −56.024 | 1.00 | 31.79 |
| ATOM | 3977 | OG1 | THR | A | 527 | 17.217 | 30.931 | −56.682 | 1.00 | 31.43 |
| ATOM | 3978 | CG2 | THR | A | 527 | 16.169 | 28.739 | −56.724 | 1.00 | 32.27 |
| ATOM | 3979 | C | THR | A | 527 | 14.911 | 32.045 | −55.152 | 1.00 | 31.99 |
| ATOM | 3980 | O | THR | A | 527 | 14.847 | 31.959 | −53.922 | 1.00 | 32.36 |
| ATOM | 3981 | N | SER | A | 528 | 14.986 | 33.209 | −55.787 | 1.00 | 31.79 |
| ATOM | 3982 | CA | SER | A | 528 | 14.928 | 34.463 | −55.054 | 1.00 | 32.02 |
| ATOM | 3983 | CB | SER | A | 528 | 15.517 | 35.615 | −55.885 | 1.00 | 32.70 |
| ATOM | 3984 | OG | SER | A | 528 | 14.712 | 35.882 | −57.031 | 1.00 | 34.94 |
| ATOM | 3985 | C | SER | A | 528 | 13.497 | 34.784 | −54.579 | 1.00 | 31.23 |
| ATOM | 3986 | O | SER | A | 528 | 13.330 | 35.435 | −53.550 | 1.00 | 32.02 |
| ATOM | 3987 | N | ALA | A | 529 | 12.479 | 34.314 | −55.306 | 1.00 | 29.33 |
| ATOM | 3988 | CA | ALA | A | 529 | 11.093 | 34.506 | −54.893 | 1.00 | 27.60 |
| ATOM | 3989 | CB | ALA | A | 529 | 10.211 | 34.864 | −56.086 | 1.00 | 27.34 |
| ATOM | 3990 | C | ALA | A | 529 | 10.482 | 33.328 | −54.112 | 1.00 | 26.40 |
| ATOM | 3991 | O | ALA | A | 529 | 9.311 | 33.382 | −53.754 | 1.00 | 26.54 |
| ATOM | 3992 | N | ALA | A | 530 | 11.268 | 32.286 | −53.842 | 1.00 | 25.12 |
| ATOM | 3993 | CA | ALA | A | 530 | 10.777 | 31.114 | −53.096 | 1.00 | 24.19 |
| ATOM | 3994 | CB | ALA | A | 530 | 11.855 | 30.063 | −52.991 | 1.00 | 23.88 |
| ATOM | 3995 | C | ALA | A | 530 | 10.336 | 31.555 | −51.706 | 1.00 | 23.55 |
| ATOM | 3996 | O | ALA | A | 530 | 10.848 | 32.540 | −51.182 | 1.00 | 23.61 |
| ATOM | 3997 | N | VAL | A | 531 | 9.396 | 30.833 | −51.110 | 1.00 | 22.18 |
| ATOM | 3998 | CA | VAL | A | 531 | 8.851 | 31.248 | −49.821 | 1.00 | 22.62 |
| ATOM | 3999 | CB | VAL | A | 531 | 7.380 | 30.821 | −49.677 | 1.00 | 22.81 |
| ATOM | 4000 | CG1 | VAL | A | 531 | 6.815 | 31.335 | −48.346 | 1.00 | 25.06 |
| ATOM | 4001 | CG2 | VAL | A | 531 | 6.551 | 31.353 | −50.886 | 1.00 | 25.38 |
| ATOM | 4002 | C | VAL | A | 531 | 9.659 | 30.646 | −48.674 | 1.00 | 21.29 |
| ATOM | 4003 | O | VAL | A | 531 | 9.768 | 29.425 | −48.564 | 1.00 | 20.72 |
| ATOM | 4004 | N | ALA | A | 532 | 10.215 | 31.493 | −47.819 | 1.00 | 20.86 |
| ATOM | 4005 | CA | ALA | A | 532 | 11.008 | 30.999 | −46.698 | 1.00 | 20.26 |
| ATOM | 4006 | CB | ALA | A | 532 | 11.850 | 32.128 | −46.084 | 1.00 | 21.02 |
| ATOM | 4007 | C | ALA | A | 532 | 10.093 | 30.356 | −45.646 | 1.00 | 20.51 |
| ATOM | 4008 | O | ALA | A | 532 | 9.019 | 30.884 | −45.337 | 1.00 | 20.05 |
| ATOM | 4009 | N | LEU | A | 533 | 10.514 | 29.200 | −45.129 | 1.00 | 19.00 |
| ATOM | 4010 | CA | LEU | A | 533 | 9.855 | 28.565 | −43.999 | 1.00 | 18.65 |
| ATOM | 4011 | CB | LEU | A | 533 | 9.901 | 27.029 | −44.148 | 1.00 | 18.14 |
| ATOM | 4012 | CG | LEU | A | 533 | 9.395 | 26.450 | −45.483 | 1.00 | 19.25 |
| ATOM | 4013 | CD1 | LEU | A | 533 | 9.385 | 24.923 | −45.427 | 1.00 | 21.28 |
| ATOM | 4014 | CD2 | LEU | A | 533 | 8.030 | 26.980 | −45.894 | 1.00 | 18.58 |
| ATOM | 4015 | C | LEU | A | 533 | 10.541 | 29.014 | −42.702 | 1.00 | 18.95 |
| ATOM | 4016 | O | LEU | A | 533 | 11.622 | 29.648 | −42.744 | 1.00 | 18.94 |
| ATOM | 4017 | N | ASP | A | 534 | 9.905 | 28.715 | −41.570 | 1.00 | 18.56 |
| ATOM | 4018 | CA | ASP | A | 534 | 10.381 | 29.096 | −40.238 | 1.00 | 18.36 |
| ATOM | 4019 | CB | ASP | A | 534 | 9.220 | 29.634 | −39.374 | 1.00 | 19.76 |
| ATOM | 4020 | CG | ASP | A | 534 | 8.757 | 30.992 | −39.798 | 1.00 | 23.55 |
| ATOM | 4021 | OD1 | ASP | A | 534 | 7.548 | 31.264 | −39.659 | 1.00 | 26.14 |
| ATOM | 4022 | OD2 | ASP | A | 534 | 9.600 | 31.774 | −40.283 | 1.00 | 27.39 |
| ATOM | 4023 | C | ASP | A | 534 | 10.877 | 27.867 | −39.504 | 1.00 | 17.68 |
| ATOM | 4024 | O | ASP | A | 534 | 10.310 | 26.780 | −39.667 | 1.00 | 16.10 |
| ATOM | 4025 | N | ALA | A | 535 | 11.883 | 28.057 | −38.654 | 1.00 | 17.05 |
| ATOM | 4026 | CA | ALA | A | 535 | 12.405 | 26.950 | −37.835 | 1.00 | 17.76 |
| ATOM | 4027 | CB | ALA | A | 535 | 13.926 | 26.952 | −37.832 | 1.00 | 17.96 |
| ATOM | 4028 | C | ALA | A | 535 | 11.872 | 27.027 | −36.403 | 1.00 | 17.82 |
| ATOM | 4029 | O | ALA | A | 535 | 12.482 | 26.490 | −35.474 | 1.00 | 18.36 |
| ATOM | 4030 | N | VAL | A | 536 | 10.745 | 27.706 | −36.225 | 1.00 | 17.68 |
| ATOM | 4031 | CA | VAL | A | 536 | 10.138 | 27.861 | −34.898 | 1.00 | 18.65 |
| ATOM | 4032 | CB | VAL | A | 536 | 8.824 | 28.719 | −34.975 | 1.00 | 18.66 |
| ATOM | 4033 | CG1 | VAL | A | 536 | 7.805 | 28.123 | −35.971 | 1.00 | 19.98 |
| ATOM | 4034 | CG2 | VAL | A | 536 | 8.208 | 28.962 | −33.570 | 1.00 | 20.19 |
| ATOM | 4035 | C | VAL | A | 536 | 9.938 | 26.514 | −34.155 | 1.00 | 18.51 |
| ATOM | 4036 | O | VAL | A | 536 | 10.124 | 26.437 | −32.923 | 1.00 | 19.61 |
| ATOM | 4037 | N | ASN | A | 537 | 9.570 | 25.468 | −34.883 | 1.00 | 18.59 |
| ATOM | 4038 | CA | ASN | A | 537 | 9.344 | 24.154 | −34.261 | 1.00 | 19.07 |
| ATOM | 4039 | CB | ASN | A | 537 | 8.074 | 23.498 | −34.816 | 1.00 | 19.28 |
| ATOM | 4040 | CG | ASN | A | 537 | 6.800 | 24.252 | −34.448 | 1.00 | 21.02 |
| ATOM | 4041 | OD1 | ASN | A | 537 | 6.742 | 24.940 | −33.435 | 1.00 | 24.12 |
| ATOM | 4042 | ND2 | ASN | A | 537 | 5.762 | 24.089 | −35.265 | 1.00 | 20.99 |
| ATOM | 4043 | C | ASN | A | 537 | 10.518 | 23.182 | −34.445 | 1.00 | 19.15 |
| ATOM | 4044 | O | ASN | A | 537 | 10.394 | 21.971 | −34.196 | 1.00 | 18.60 |
| ATOM | 4045 | N | TYR | A | 538 | 11.653 | 23.699 | −34.897 | 1.00 | 19.05 |
| ATOM | 4046 | CA | TYR | A | 538 | 12.767 | 22.830 | −35.234 | 1.00 | 20.32 |
| ATOM | 4047 | CB | TYR | A | 538 | 13.816 | 23.618 | −36.026 | 1.00 | 20.37 |
| ATOM | 4048 | CG | TYR | A | 538 | 14.916 | 22.747 | −36.588 | 1.00 | 20.37 |
| ATOM | 4049 | CD1 | TYR | A | 538 | 14.822 | 22.238 | −37.886 | 1.00 | 20.39 |
| ATOM | 4050 | CE1 | TYR | A | 538 | 15.853 | 21.436 | −38.437 | 1.00 | 20.49 |
| ATOM | 4051 | CZ | TYR | A | 538 | 16.961 | 21.137 | −37.670 | 1.00 | 20.65 |
| ATOM | 4052 | OH | TYR | A | 538 | 17.946 | 20.341 | −38.218 | 1.00 | 21.53 |
| ATOM | 4053 | CE2 | TYR | A | 538 | 17.066 | 21.602 | −36.361 | 1.00 | 22.05 |
| ATOM | 4054 | CD2 | TYR | A | 538 | 16.043 | 22.418 | −35.825 | 1.00 | 21.95 |
| ATOM | 4055 | C | TYR | A | 538 | 13.436 | 22.209 | −33.981 | 1.00 | 21.29 |
| ATOM | 4056 | O | TYR | A | 538 | 13.733 | 22.919 | −33.036 | 1.00 | 21.89 |
| ATOM | 4057 | N | ALA | A | 539 | 13.695 | 20.902 | −34.014 | 1.00 | 22.15 |
| ATOM | 4058 | CA | ALA | A | 539 | 14.646 | 20.258 | −33.083 | 1.00 | 23.67 |
| ATOM | 4059 | CB | ALA | A | 539 | 13.909 | 19.536 | −31.976 | 1.00 | 23.94 |
| ATOM | 4060 | C | ALA | A | 539 | 15.545 | 19.289 | −33.849 | 1.00 | 24.35 |
| ATOM | 4061 | O | ALA | A | 539 | 15.117 | 18.698 | −34.833 | 1.00 | 23.63 |
| ATOM | 4062 | N | ASP | A | 540 | 16.793 | 19.118 | −33.405 | 1.00 | 25.69 |
| ATOM | 4063 | CA | ASP | A | 540 | 17.722 | 18.196 | −34.099 | 1.00 | 27.42 |
| ATOM | 4064 | CB | ASP | A | 540 | 19.044 | 18.051 | −33.339 | 1.00 | 28.62 |
| ATOM | 4065 | CG | ASP | A | 540 | 19.724 | 19.368 | −33.140 | 1.00 | 33.80 |
| ATOM | 4066 | OD1 | ASP | A | 540 | 19.875 | 20.115 | −34.147 | 1.00 | 36.83 |
| ATOM | 4067 | OD2 | ASP | A | 540 | 20.080 | 19.663 | −31.970 | 1.00 | 40.32 |
| ATOM | 4068 | C | ASP | A | 540 | 17.150 | 16.818 | −34.400 | 1.00 | 26.63 |
| ATOM | 4069 | O | ASP | A | 540 | 17.386 | 16.277 | −35.485 | 1.00 | 27.28 |
| ATOM | 4070 | N | ASN | A | 541 | 16.403 | 16.247 | −33.458 | 1.00 | 25.08 |
| ATOM | 4071 | CA | ASN | A | 541 | 15.827 | 14.922 | −33.687 | 1.00 | 24.03 |
| ATOM | 4072 | CB | ASN | A | 541 | 15.988 | 14.032 | −32.441 | 1.00 | 25.14 |
| ATOM | 4073 | CG | ASN | A | 541 | 15.337 | 14.623 | −31.191 | 1.00 | 27.81 |
| ATOM | 4074 | OD1 | ASN | A | 541 | 15.366 | 14.001 | −30.118 | 1.00 | 31.57 |
| ATOM | 4075 | ND2 | ASN | A | 541 | 14.771 | 15.824 | −31.306 | 1.00 | 28.81 |
| ATOM | 4076 | C | ASN | A | 541 | 14.349 | 14.991 | −34.118 | 1.00 | 22.46 |
| ATOM | 4077 | O | ASN | A | 541 | 13.660 | 13.979 | −34.172 | 1.00 | 22.35 |
| ATOM | 4078 | N | HIS | A | 542 | 13.871 | 16.197 | −34.403 | 1.00 | 19.85 |
| ATOM | 4079 | CA | HIS | A | 542 | 12.580 | 16.354 | −35.083 | 1.00 | 18.22 |
| ATOM | 4080 | CB | HIS | A | 542 | 11.426 | 16.392 | −34.062 | 1.00 | 16.89 |
| ATOM | 4081 | CG | HIS | A | 542 | 10.071 | 16.341 | −34.699 | 1.00 | 17.32 |
| ATOM | 4082 | ND1 | HIS | A | 542 | 9.211 | 17.417 | −34.711 | 1.00 | 16.28 |
| ATOM | 4083 | CE1 | HIS | A | 542 | 8.111 | 17.088 | −35.371 | 1.00 | 14.64 |
| ATOM | 4084 | NE2 | HIS | A | 542 | 8.217 | 15.834 | −35.765 | 1.00 | 15.41 |
| ATOM | 4085 | CD2 | HIS | A | 542 | 9.435 | 15.340 | −35.358 | 1.00 | 15.14 |
| ATOM | 4086 | C | HIS | A | 542 | 12.662 | 17.650 | −35.902 | 1.00 | 17.48 |
| ATOM | 4087 | O | HIS | A | 542 | 12.198 | 18.698 | −35.446 | 1.00 | 17.79 |
| ATOM | 4088 | N | PRO | A | 543 | 13.324 | 17.584 | −37.083 | 1.00 | 17.00 |
| ATOM | 4089 | CA | PRO | A | 543 | 13.797 | 18.752 | −37.832 | 1.00 | 17.09 |
| ATOM | 4090 | CB | PRO | A | 543 | 14.948 | 18.164 | −38.677 | 1.00 | 16.57 |
| ATOM | 4091 | CG | PRO | A | 543 | 14.472 | 16.759 | −38.994 | 1.00 | 18.22 |
| ATOM | 4092 | CD | PRO | A | 543 | 13.676 | 16.321 | −37.764 | 1.00 | 17.39 |
| ATOM | 4093 | C | PRO | A | 543 | 12.718 | 19.435 | −38.691 | 1.00 | 16.46 |
| ATOM | 4094 | O | PRO | A | 543 | 12.811 | 19.497 | −39.922 | 1.00 | 17.09 |
| ATOM | 4095 | N | LEU | A | 544 | 11.726 | 19.987 | −38.009 | 1.00 | 15.36 |
| ATOM | 4096 | CA | LEU | A | 544 | 10.534 | 20.535 | −38.636 | 1.00 | 15.10 |
| ATOM | 4097 | CB | LEU | A | 544 | 9.341 | 20.376 | −37.672 | 1.00 | 15.29 |
| ATOM | 4098 | CG | LEU | A | 544 | 7.968 | 20.927 | −38.134 | 1.00 | 16.53 |
| ATOM | 4099 | CD1 | LEU | A | 544 | 7.524 | 20.364 | −39.494 | 1.00 | 15.92 |
| ATOM | 4100 | CD2 | LEU | A | 544 | 6.900 | 20.700 | −37.062 | 1.00 | 14.59 |
| ATOM | 4101 | C | LEU | A | 544 | 10.694 | 22.018 | −39.025 | 1.00 | 15.09 |
| ATOM | 4102 | O | LEU | A | 544 | 11.037 | 22.851 | −38.197 | 1.00 | 15.34 |
| ATOM | 4103 | N | TRP | A | 545 | 10.456 | 22.298 | −40.303 | 1.00 | 15.03 |
| ATOM | 4104 | CA | TRP | A | 545 | 10.327 | 23.637 | −40.843 | 1.00 | 15.36 |
| ATOM | 4105 | CB | TRP | A | 545 | 11.288 | 23.790 | −42.023 | 1.00 | 15.40 |
| ATOM | 4106 | CG | TRP | A | 545 | 12.758 | 23.921 | −41.663 | 1.00 | 15.62 |
| ATOM | 4107 | CD1 | TRP | A | 545 | 13.653 | 22.903 | −41.384 | 1.00 | 16.85 |
| ATOM | 4108 | NE1 | TRP | A | 545 | 14.906 | 23.437 | −41.129 | 1.00 | 18.69 |
| ATOM | 4109 | CE2 | TRP | A | 545 | 14.837 | 24.803 | −41.246 | 1.00 | 17.78 |
| ATOM | 4110 | CD2 | TRP | A | 545 | 13.498 | 25.140 | −41.584 | 1.00 | 17.01 |
| ATOM | 4111 | CE3 | TRP | A | 545 | 13.163 | 26.488 | −41.777 | 1.00 | 17.17 |
| ATOM | 4112 | CZ3 | TRP | A | 545 | 14.165 | 27.456 | −41.637 | 1.00 | 18.29 |
| ATOM | 4113 | CH2 | TRP | A | 545 | 15.483 | 27.085 | −41.295 | 1.00 | 17.22 |
| ATOM | 4114 | CZ2 | TRP | A | 545 | 15.835 | 25.767 | −41.111 | 1.00 | 19.09 |
| ATOM | 4115 | C | TRP | A | 545 | 8.907 | 23.832 | −41.359 | 1.00 | 15.14 |
| ATOM | 4116 | O | TRP | A | 545 | 8.327 | 22.933 | −41.986 | 1.00 | 14.45 |
| ATOM | 4117 | N | ILE | A | 546 | 8.362 | 25.025 | −41.149 | 1.00 | 15.19 |
| ATOM | 4118 | CA | ILE | A | 546 | 6.938 | 25.244 | −41.428 | 1.00 | 16.51 |
| ATOM | 4119 | CB | ILE | A | 546 | 6.107 | 24.988 | −40.130 | 1.00 | 17.39 |
| ATOM | 4120 | CG1 | ILE | A | 546 | 4.615 | 24.852 | −40.420 | 1.00 | 20.44 |
| ATOM | 4121 | CD1 | ILE | A | 546 | 3.882 | 23.992 | −39.392 | 1.00 | 23.59 |
| ATOM | 4122 | CG2 | ILE | A | 546 | 6.391 | 26.064 | −39.050 | 1.00 | 17.70 |
| ATOM | 4123 | C | ILE | A | 546 | 6.674 | 26.635 | −42.006 | 1.00 | 16.75 |
| ATOM | 4124 | O | ILE | A | 546 | 7.352 | 27.593 | −41.647 | 1.00 | 15.81 |
| ATOM | 4125 | N | ALA | A | 547 | 5.716 | 26.743 | −42.925 | 1.00 | 16.88 |
| ATOM | 4126 | CA | ALA | A | 547 | 5.197 | 28.057 | −43.279 | 1.00 | 17.94 |
| ATOM | 4127 | CB | ALA | A | 547 | 6.222 | 28.893 | −43.931 | 1.00 | 21.49 |
| ATOM | 4128 | C | ALA | A | 547 | 4.009 | 27.919 | −44.167 | 1.00 | 18.29 |
| ATOM | 4129 | O | ALA | A | 547 | 3.727 | 26.828 | −44.655 | 1.00 | 18.52 |
| ATOM | 4130 | N | THR | A | 548 | 3.316 | 29.031 | −44.362 | 1.00 | 17.99 |
| ATOM | 4131 | CA | THR | A | 548 | 1.970 | 29.017 | −44.919 | 1.00 | 18.72 |
| ATOM | 4132 | CB | THR | A | 548 | 0.929 | 29.419 | −43.855 | 1.00 | 18.65 |
| ATOM | 4133 | OG1 | THR | A | 548 | 1.000 | 28.500 | −42.751 | 1.00 | 19.46 |
| ATOM | 4134 | CG2 | THR | A | 548 | −0.491 | 29.379 | −44.438 | 1.00 | 18.79 |
| ATOM | 4135 | C | THR | A | 548 | 1.865 | 29.960 | −46.104 | 1.00 | 19.60 |
| ATOM | 4136 | O | THR | A | 548 | 2.347 | 31.090 | −46.040 | 1.00 | 19.83 |
| ATOM | 4137 | N | VAL | A | 549 | 1.227 | 29.485 | −47.164 | 1.00 | 20.41 |
| ATOM | 4138 | CA | VAL | A | 549 | 1.048 | 30.280 | −48.389 | 1.00 | 22.36 |
| ATOM | 4139 | CB | VAL | A | 549 | 1.944 | 29.722 | −49.537 | 1.00 | 22.77 |
| ATOM | 4140 | CG1 | VAL | A | 549 | 1.717 | 30.491 | −50.845 | 1.00 | 25.80 |
| ATOM | 4141 | CG2 | VAL | A | 549 | 3.429 | 29.781 | −49.148 | 1.00 | 24.78 |
| ATOM | 4142 | C | VAL | A | 549 | −0.399 | 30.119 | −48.800 | 1.00 | 22.21 |
| ATOM | 4143 | O | VAL | A | 549 | −0.943 | 29.018 | −48.719 | 1.00 | 21.56 |
| ATOM | 4144 | N | ASN | A | 550 | −1.028 | 31.211 | −49.240 | 1.00 | 22.80 |
| ATOM | 4145 | CA | ASN | A | 550 | −2.356 | 31.107 | −49.831 | 1.00 | 23.95 |
| ATOM | 4146 | CB | ASN | A | 550 | −3.114 | 32.411 | −49.649 | 1.00 | 24.39 |
| ATOM | 4147 | CG | ASN | A | 550 | −3.367 | 32.706 | −48.201 | 1.00 | 27.42 |
| ATOM | 4148 | OD1 | ASN | A | 550 | −3.811 | 31.838 | −47.462 | 1.00 | 28.69 |
| ATOM | 4149 | ND2 | ASN | A | 550 | −3.041 | 33.911 | −47.771 | 1.00 | 31.89 |
| ATOM | 4150 | C | ASN | A | 550 | −2.278 | 30.733 | −51.294 | 1.00 | 23.98 |
| ATOM | 4151 | O | ASN | A | 550 | −1.598 | 31.400 | −52.065 | 1.00 | 24.73 |
| ATOM | 4152 | N | LEU | A | 551 | −2.973 | 29.667 | −51.662 | 1.00 | 24.24 |
| ATOM | 4153 | CA | LEU | A | 551 | −3.016 | 29.180 | −53.020 | 1.00 | 24.78 |
| ATOM | 4154 | CB | LEU | A | 551 | −2.348 | 27.797 | −53.135 | 1.00 | 25.00 |
| ATOM | 4155 | CG | LEU | A | 551 | −0.858 | 27.721 | −52.787 | 1.00 | 25.19 |
| ATOM | 4156 | CD1 | LEU | A | 551 | −0.356 | 26.284 | −52.803 | 1.00 | 27.45 |
| ATOM | 4157 | CD2 | LEU | A | 551 | −0.018 | 28.613 | −53.718 | 1.00 | 26.73 |
| ATOM | 4158 | C | LEU | A | 551 | −4.471 | 29.104 | −53.488 | 1.00 | 25.46 |
| ATOM | 4159 | O | LEU | A | 551 | −5.393 | 29.018 | −52.675 | 1.00 | 24.27 |
| ATOM | 4160 | N | GLU | A | 552 | −4.661 | 29.148 | −54.804 | 1.00 | 26.15 |
| ATOM | 4161 | CA | GLU | A | 552 | −6.004 | 29.165 | −55.366 | 1.00 | 28.20 |
| ATOM | 4162 | CB | GLU | A | 552 | −5.955 | 29.639 | −56.823 | 1.00 | 28.30 |
| ATOM | 4163 | CG | GLU | A | 552 | −7.326 | 29.739 | −57.494 | 1.00 | 32.28 |
| ATOM | 4164 | CD | GLU | A | 552 | −7.250 | 30.281 | −58.926 | 1.00 | 33.44 |
| ATOM | 4165 | OE1 | GLU | A | 552 | −8.110 | 31.126 | −59.274 | 1.00 | 41.80 |
| ATOM | 4166 | OE2 | GLU | A | 552 | −6.340 | 29.873 | −59.695 | 1.00 | 39.14 |
| ATOM | 4167 | C | GLU | A | 552 | −6.610 | 27.768 | −55.253 | 1.00 | 26.97 |
| ATOM | 4168 | O | GLU | A | 552 | −5.979 | 26.783 | −55.622 | 1.00 | 26.49 |
| ATOM | 4169 | N | ALA | A | 553 | −7.822 | 27.684 | −54.723 | 1.00 | 27.21 |
| ATOM | 4170 | CA | ALA | A | 553 | −8.502 | 26.399 | −54.603 | 1.00 | 27.89 |
| ATOM | 4171 | CB | ALA | A | 553 | −9.876 | 26.574 | −53.953 | 1.00 | 28.37 |
| ATOM | 4172 | C | ALA | A | 553 | −8.637 | 25.773 | −55.979 | 1.00 | 28.57 |
| ATOM | 4173 | O | ALA | A | 553 | −8.900 | 26.477 | −56.952 | 1.00 | 28.83 |
| ATOM | 4174 | N | GLY | A | 554 | −8.438 | 24.465 | −56.064 | 1.00 | 28.44 |
| ATOM | 4175 | CA | GLY | A | 554 | −8.556 | 23.753 | −57.330 | 1.00 | 29.66 |
| ATOM | 4176 | C | GLY | A | 554 | −7.274 | 23.693 | −58.145 | 1.00 | 30.04 |
| ATOM | 4177 | O | GLY | A | 554 | −7.122 | 22.814 | −59.000 | 1.00 | 30.33 |
| ATOM | 4178 | N | ASP | A | 555 | −6.347 | 24.606 | −57.869 | 1.00 | 30.23 |
| ATOM | 4179 | CA | ASP | A | 555 | −5.098 | 24.716 | −58.630 | 1.00 | 30.61 |
| ATOM | 4180 | CB | ASP | A | 555 | −4.313 | 25.939 | −58.161 | 1.00 | 30.85 |
| ATOM | 4181 | CG | ASP | A | 555 | −3.382 | 26.503 | −59.236 | 1.00 | 34.49 |
| ATOM | 4182 | OD1 | ASP | A | 555 | −3.441 | 26.053 | −60.407 | 1.00 | 37.19 |
| ATOM | 4183 | OD2 | ASP | A | 555 | −2.572 | 27.408 | −58.901 | 1.00 | 36.59 |
| ATOM | 4184 | C | ASP | A | 555 | −4.238 | 23.467 | −58.486 | 1.00 | 30.14 |
| ATOM | 4185 | O | ASP | A | 555 | −4.156 | 22.882 | −57.419 | 1.00 | 30.07 |
| ATOM | 4186 | N | VAL | A | 556 | −3.602 | 23.046 | −59.572 | 1.00 | 29.55 |
| ATOM | 4187 | CA | VAL | A | 556 | −2.628 | 21.963 | −59.492 | 1.00 | 28.74 |
| ATOM | 4188 | CB | VAL | A | 556 | −2.732 | 20.987 | −60.680 | 1.00 | 29.35 |
| ATOM | 4189 | CG1 | VAL | A | 556 | −1.666 | 19.877 | −60.569 | 1.00 | 28.99 |
| ATOM | 4190 | CG2 | VAL | A | 556 | −4.125 | 20.365 | −60.720 | 1.00 | 30.44 |
| ATOM | 4191 | C | VAL | A | 556 | −1.261 | 22.623 | −59.448 | 1.00 | 28.04 |
| ATOM | 4192 | O | VAL | A | 556 | −0.869 | 23.336 | −60.384 | 1.00 | 27.25 |
| ATOM | 4193 | N | VAL | A | 557 | −0.544 | 22.389 | −58.352 | 1.00 | 26.81 |
| ATOM | 4194 | CA | VAL | A | 557 | 0.739 | 23.040 | −58.100 | 1.00 | 26.14 |
| ATOM | 4195 | CB | VAL | A | 557 | 0.690 | 23.859 | −56.759 | 1.00 | 26.71 |
| ATOM | 4196 | CG1 | VAL | A | 557 | 2.073 | 24.140 | −56.219 | 1.00 | 27.82 |
| ATOM | 4197 | CG2 | VAL | A | 557 | −0.088 | 25.175 | −56.952 | 1.00 | 27.34 |
| ATOM | 4198 | C | VAL | A | 557 | 1.856 | 21.999 | −58.092 | 1.00 | 25.64 |
| ATOM | 4199 | O | VAL | A | 557 | 1.646 | 20.845 | −57.693 | 1.00 | 25.24 |
| ATOM | 4200 | N | GLU | A | 558 | 3.035 | 22.409 | −58.553 | 1.00 | 24.16 |
| ATOM | 4201 | CA | GLU | A | 558 | 4.223 | 21.579 | −58.516 | 1.00 | 24.38 |
| ATOM | 4202 | CB | GLU | A | 558 | 4.737 | 21.296 | −59.933 | 1.00 | 24.86 |
| ATOM | 4203 | CG | GLU | A | 558 | 4.064 | 20.108 | −60.606 | 1.00 | 26.34 |
| ATOM | 4204 | CD | GLU | A | 558 | 4.670 | 19.790 | −61.962 | 1.00 | 27.68 |
| ATOM | 4205 | OE1 | GLU | A | 558 | 5.917 | 19.684 | −62.065 | 1.00 | 30.56 |
| ATOM | 4206 | OE2 | GLU | A | 558 | 3.883 | 19.638 | −62.915 | 1.00 | 32.45 |
| ATOM | 4207 | C | GLU | A | 558 | 5.262 | 22.337 | −57.730 | 1.00 | 22.54 |
| ATOM | 4208 | O | GLU | A | 558 | 5.389 | 23.550 | −57.883 | 1.00 | 22.93 |
| ATOM | 4209 | N | TYR | A | 559 | 5.992 | 21.640 | −56.867 | 1.00 | 21.45 |
| ATOM | 4210 | CA | TYR | A | 559 | 6.927 | 22.346 | −55.995 | 1.00 | 19.96 |
| ATOM | 4211 | CB | TYR | A | 559 | 6.188 | 22.972 | −54.784 | 1.00 | 19.42 |
| ATOM | 4212 | CG | TYR | A | 559 | 5.624 | 21.952 | −53.796 | 1.00 | 18.65 |
| ATOM | 4213 | CD1 | TYR | A | 559 | 6.383 | 21.524 | −52.703 | 1.00 | 18.64 |
| ATOM | 4214 | CE1 | TYR | A | 559 | 5.887 | 20.595 | −51.794 | 1.00 | 19.09 |
| ATOM | 4215 | CZ | TYR | A | 559 | 4.614 | 20.090 | −51.955 | 1.00 | 18.58 |
| ATOM | 4216 | OH | TYR | A | 559 | 4.135 | 19.160 | −51.056 | 1.00 | 20.55 |
| ATOM | 4217 | CE2 | TYR | A | 559 | 3.819 | 20.493 | −53.024 | 1.00 | 18.22 |
| ATOM | 4218 | CD2 | TYR | A | 559 | 4.335 | 21.438 | −53.946 | 1.00 | 18.82 |
| ATOM | 4219 | C | TYR | A | 559 | 8.066 | 21.445 | −55.541 | 1.00 | 20.13 |
| ATOM | 4220 | O | TYR | A | 559 | 8.008 | 20.215 | −55.679 | 1.00 | 20.26 |
| ATOM | 4221 | N | LYS | A | 560 | 9.098 | 22.079 | −54.995 | 1.00 | 19.51 |
| ATOM | 4222 | CA | LYS | A | 560 | 10.208 | 21.379 | −54.349 | 1.00 | 19.72 |
| ATOM | 4223 | CB | LYS | A | 560 | 11.410 | 21.175 | −55.282 | 1.00 | 19.66 |
| ATOM | 4224 | CG | LYS | A | 560 | 11.390 | 19.870 | −56.058 | 1.00 | 22.20 |
| ATOM | 4225 | CD | LYS | A | 560 | 12.767 | 19.633 | −56.714 | 1.00 | 24.46 |
| ATOM | 4226 | CE | LYS | A | 560 | 12.781 | 18.341 | −57.531 | 1.00 | 26.96 |
| ATOM | 4227 | NZ | LYS | A | 560 | 14.189 | 18.050 | −57.980 | 1.00 | 26.97 |
| ATOM | 4228 | C | LYS | A | 560 | 10.680 | 22.257 | −53.234 | 1.00 | 18.68 |
| ATOM | 4229 | O | LYS | A | 560 | 10.583 | 23.484 | −53.318 | 1.00 | 19.68 |
| ATOM | 4230 | N | TYR | A | 561 | 11.240 | 21.640 | −52.206 | 1.00 | 17.87 |
| ATOM | 4231 | CA | TYR | A | 561 | 11.927 | 22.420 | −51.187 | 1.00 | 17.82 |
| ATOM | 4232 | CB | TYR | A | 561 | 11.921 | 21.690 | −49.840 | 1.00 | 17.42 |
| ATOM | 4233 | CG | TYR | A | 561 | 10.518 | 21.449 | −49.346 | 1.00 | 16.12 |
| ATOM | 4234 | CD1 | TYR | A | 561 | 9.831 | 20.276 | −49.661 | 1.00 | 15.94 |
| ATOM | 4235 | CE1 | TYR | A | 561 | 8.511 | 20.062 | −49.199 | 1.00 | 15.79 |
| ATOM | 4236 | CZ | TYR | A | 561 | 7.897 | 21.050 | −48.456 | 1.00 | 15.89 |
| ATOM | 4237 | OH | TYR | A | 561 | 6.614 | 20.889 | −47.981 | 1.00 | 17.17 |
| ATOM | 4238 | CE2 | TYR | A | 561 | 8.557 | 22.224 | −48.164 | 1.00 | 16.19 |
| ATOM | 4239 | CD2 | TYR | A | 561 | 9.856 | 22.430 | −48.625 | 1.00 | 17.57 |
| ATOM | 4240 | C | TYR | A | 561 | 13.360 | 22.650 | −51.607 | 1.00 | 18.63 |
| ATOM | 4241 | O | TYR | A | 561 | 13.963 | 21.786 | −52.265 | 1.00 | 18.40 |
| ATOM | 4242 | N | ILE | A | 562 | 13.904 | 23.792 | −51.201 | 1.00 | 18.56 |
| ATOM | 4243 | CA | ILE | A | 562 | 15.322 | 24.085 | −51.434 | 1.00 | 20.18 |
| ATOM | 4244 | CB | ILE | A | 562 | 15.524 | 25.247 | −52.419 | 1.00 | 19.65 |
| ATOM | 4245 | CG1 | ILE | A | 562 | 14.837 | 26.520 | −51.896 | 1.00 | 21.64 |
| ATOM | 4246 | CD1 | ILE | A | 562 | 15.074 | 27.789 | −52.741 | 1.00 | 21.24 |
| ATOM | 4247 | CG2 | ILE | A | 562 | 15.017 | 24.829 | −53.797 | 1.00 | 20.44 |
| ATOM | 4248 | C | ILE | A | 562 | 15.971 | 24.446 | −50.128 | 1.00 | 20.56 |
| ATOM | 4249 | O | ILE | A | 562 | 15.316 | 24.956 | −49.229 | 1.00 | 19.84 |
| ATOM | 4250 | N | ASN | A | 563 | 17.254 | 24.134 | −50.029 | 1.00 | 21.41 |
| ATOM | 4251 | CA | ASN | A | 563 | 18.076 | 24.467 | −48.886 | 1.00 | 22.99 |
| ATOM | 4252 | CB | ASN | A | 563 | 18.833 | 23.209 | −48.435 | 1.00 | 22.69 |
| ATOM | 4253 | CG | ASN | A | 563 | 19.629 | 23.433 | −47.156 | 1.00 | 25.48 |
| ATOM | 4254 | OD1 | ASN | A | 563 | 20.203 | 24.492 | −46.965 | 1.00 | 28.81 |
| ATOM | 4255 | ND2 | ASN | A | 563 | 19.669 | 22.436 | −46.285 | 1.00 | 25.82 |
| ATOM | 4256 | C | ASN | A | 563 | 19.039 | 25.561 | −49.372 | 1.00 | 24.66 |
| ATOM | 4257 | O | ASN | A | 563 | 19.794 | 25.326 | −50.323 | 1.00 | 24.16 |
| ATOM | 4258 | N | VAL | A | 564 | 18.977 | 26.749 | −48.780 | 1.00 | 26.20 |
| ATOM | 4259 | CA | VAL | A | 564 | 19.877 | 27.837 | −49.206 | 1.00 | 28.74 |
| ATOM | 4260 | CB | VAL | A | 564 | 19.156 | 29.091 | −49.832 | 1.00 | 28.91 |
| ATOM | 4261 | CG1 | VAL | A | 564 | 19.461 | 30.408 | −49.087 | 1.00 | 31.10 |
| ATOM | 4262 | CG2 | VAL | A | 564 | 17.655 | 28.836 | −50.079 | 1.00 | 28.92 |
| ATOM | 4263 | C | VAL | A | 564 | 20.886 | 28.181 | −48.122 | 1.00 | 29.56 |
| ATOM | 4264 | O | VAL | A | 564 | 20.538 | 28.320 | −46.954 | 1.00 | 29.10 |
| ATOM | 4265 | N | GLY | A | 565 | 22.150 | 28.266 | −48.527 | 1.00 | 32.02 |
| ATOM | 4266 | CA | GLY | A | 565 | 23.252 | 28.423 | −47.577 | 1.00 | 34.92 |
| ATOM | 4267 | C | GLY | A | 565 | 23.539 | 29.876 | −47.248 | 1.00 | 37.47 |
| ATOM | 4268 | O | GLY | A | 565 | 22.969 | 30.788 | −47.871 | 1.00 | 37.66 |
| ATOM | 4269 | N | GLN | A | 566 | 24.419 | 30.098 | −46.267 | 1.00 | 39.85 |
| ATOM | 4270 | CA | GLN | A | 566 | 24.897 | 31.456 | −45.926 | 1.00 | 42.75 |
| ATOM | 4271 | CB | GLN | A | 566 | 26.054 | 31.398 | −44.918 | 1.00 | 42.83 |
| ATOM | 4272 | CG | GLN | A | 566 | 25.727 | 30.761 | −43.565 | 1.00 | 44.99 |
| ATOM | 4273 | CD | GLN | A | 566 | 26.940 | 30.689 | −42.626 | 1.00 | 44.88 |
| ATOM | 4274 | OE1 | GLN | A | 566 | 27.972 | 30.089 | −42.958 | 1.00 | 47.67 |
| ATOM | 4275 | NE2 | GLN | A | 566 | 26.810 | 31.293 | −41.441 | 1.00 | 47.62 |
| ATOM | 4276 | C | GLN | A | 566 | 25.373 | 32.195 | −47.181 | 1.00 | 43.30 |
| ATOM | 4277 | O | GLN | A | 566 | 25.052 | 33.365 | −47.389 | 1.00 | 44.01 |
| ATOM | 4278 | N | ASP | A | 567 | 26.118 | 31.479 | −48.023 | 1.00 | 44.30 |
| ATOM | 4279 | CA | ASP | A | 567 | 26.739 | 32.029 | −49.236 | 1.00 | 44.62 |
| ATOM | 4280 | CB | ASP | A | 567 | 27.916 | 31.139 | −49.650 | 1.00 | 45.13 |
| ATOM | 4281 | CG | ASP | A | 567 | 27.492 | 29.702 | −49.966 | 1.00 | 47.62 |
| ATOM | 4282 | OD1 | ASP | A | 567 | 26.421 | 29.255 | −49.485 | 1.00 | 48.73 |
| ATOM | 4283 | OD2 | ASP | A | 567 | 28.245 | 29.010 | −50.693 | 1.00 | 50.47 |
| ATOM | 4284 | C | ASP | A | 567 | 25.776 | 32.197 | −50.421 | 1.00 | 44.04 |
| ATOM | 4285 | O | ASP | A | 567 | 26.196 | 32.575 | −51.522 | 1.00 | 44.36 |
| ATOM | 4286 | N | GLY | A | 568 | 24.497 | 31.899 | −50.205 | 1.00 | 42.85 |
| ATOM | 4287 | CA | GLY | A | 568 | 23.488 | 32.045 | −51.247 | 1.00 | 41.38 |
| ATOM | 4288 | C | GLY | A | 568 | 23.359 | 30.851 | −52.177 | 1.00 | 40.41 |
| ATOM | 4289 | O | GLY | A | 568 | 22.496 | 30.854 | −53.054 | 1.00 | 40.62 |
| ATOM | 4290 | N | SER | A | 569 | 24.195 | 29.827 | −51.990 | 1.00 | 39.02 |
| ATOM | 4291 | CA | SER | A | 569 | 24.137 | 28.623 | −52.827 | 1.00 | 37.72 |
| ATOM | 4292 | CB | SER | A | 569 | 25.365 | 27.746 | −52.600 | 1.00 | 38.07 |
| ATOM | 4293 | OG | SER | A | 569 | 25.454 | 27.359 | −51.238 | 1.00 | 39.18 |
| ATOM | 4294 | C | SER | A | 569 | 22.868 | 27.819 | −52.540 | 1.00 | 36.72 |
| ATOM | 4295 | O | SER | A | 569 | 22.474 | 27.672 | −51.382 | 1.00 | 36.47 |
| ATOM | 4296 | N | VAL | A | 570 | 22.222 | 27.313 | −53.583 | 1.00 | 35.25 |
| ATOM | 4297 | CA | VAL | A | 570 | 20.988 | 26.558 | −53.365 | 1.00 | 34.26 |
| ATOM | 4298 | CB | VAL | A | 570 | 19.709 | 27.243 | −53.987 | 1.00 | 34.42 |
| ATOM | 4299 | CG1 | VAL | A | 570 | 18.992 | 26.358 | −55.020 | 1.00 | 35.11 |
| ATOM | 4300 | CG2 | VAL | A | 570 | 20.010 | 28.651 | −54.506 | 1.00 | 35.12 |
| ATOM | 4301 | C | VAL | A | 570 | 21.113 | 25.080 | −53.718 | 1.00 | 33.29 |
| ATOM | 4302 | O | VAL | A | 570 | 21.735 | 24.694 | −54.714 | 1.00 | 32.88 |
| ATOM | 4303 | N | THR | A | 571 | 20.515 | 24.261 | −52.864 | 1.00 | 31.66 |
| ATOM | 4304 | CA | THR | A | 571 | 20.480 | 22.825 | −53.021 | 1.00 | 30.87 |
| ATOM | 4305 | CB | THR | A | 571 | 21.016 | 22.146 | −51.752 | 1.00 | 30.96 |
| ATOM | 4306 | OG1 | THR | A | 571 | 22.311 | 22.686 | −51.442 | 1.00 | 33.18 |
| ATOM | 4307 | CG2 | THR | A | 571 | 21.117 | 20.637 | −51.935 | 1.00 | 30.95 |
| ATOM | 4308 | C | THR | A | 571 | 19.018 | 22.473 | −53.210 | 1.00 | 29.72 |
| ATOM | 4309 | O | THR | A | 571 | 18.186 | 22.832 | −52.373 | 1.00 | 28.84 |
| ATOM | 4310 | N | TRP | A | 572 | 18.697 | 21.846 | −54.337 | 1.00 | 29.01 |
| ATOM | 4311 | CA | TRP | A | 572 | 17.331 | 21.388 | −54.589 | 1.00 | 28.49 |
| ATOM | 4312 | CB | TRP | A | 572 | 17.004 | 21.367 | −56.086 | 1.00 | 28.99 |
| ATOM | 4313 | CG | TRP | A | 572 | 16.950 | 22.690 | −56.739 | 1.00 | 29.65 |
| ATOM | 4314 | CD1 | TRP | A | 572 | 18.014 | 23.419 | −57.217 | 1.00 | 30.67 |
| ATOM | 4315 | NE1 | TRP | A | 572 | 17.564 | 24.603 | −57.769 | 1.00 | 31.84 |
| ATOM | 4316 | CE2 | TRP | A | 572 | 16.196 | 24.655 | −57.668 | 1.00 | 30.45 |
| ATOM | 4317 | CD2 | TRP | A | 572 | 15.770 | 23.464 | −57.028 | 1.00 | 30.30 |
| ATOM | 4318 | CE3 | TRP | A | 572 | 14.398 | 23.274 | −56.791 | 1.00 | 29.43 |
| ATOM | 4319 | CZ3 | TRP | A | 572 | 13.502 | 24.266 | −57.205 | 1.00 | 30.24 |
| ATOM | 4320 | CH2 | TRP | A | 572 | 13.959 | 25.437 | −57.846 | 1.00 | 30.31 |
| ATOM | 4321 | CZ2 | TRP | A | 572 | 15.298 | 25.649 | −58.083 | 1.00 | 29.71 |
| ATOM | 4322 | C | TRP | A | 572 | 17.205 | 19.991 | −54.031 | 1.00 | 28.21 |
| ATOM | 4323 | O | TRP | A | 572 | 18.168 | 19.212 | −54.060 | 1.00 | 27.50 |
| ATOM | 4324 | N | GLU | A | 573 | 16.033 | 19.647 | −53.499 | 1.00 | 27.69 |
| ATOM | 4325 | CA | GLU | A | 573 | 15.819 | 18.251 | −53.123 | 1.00 | 27.17 |
| ATOM | 4326 | CB | GLU | A | 573 | 14.586 | 18.074 | −52.222 | 1.00 | 27.18 |
| ATOM | 4327 | CG | GLU | A | 573 | 13.287 | 18.406 | −52.901 | 1.00 | 26.04 |
| ATOM | 4328 | CD | GLU | A | 573 | 12.059 | 18.111 | −52.028 | 1.00 | 26.20 |
| ATOM | 4329 | OE1 | GLU | A | 573 | 12.112 | 17.224 | −51.141 | 1.00 | 25.84 |
| ATOM | 4330 | OE2 | GLU | A | 573 | 11.032 | 18.764 | −52.264 | 1.00 | 22.24 |
| ATOM | 4331 | C | GLU | A | 573 | 15.725 | 17.419 | −54.405 | 1.00 | 27.91 |
| ATOM | 4332 | O | GLU | A | 573 | 15.498 | 17.957 | −55.497 | 1.00 | 26.94 |
| ATOM | 4333 | N | SER | A | 574 | 15.907 | 16.108 | −54.267 | 1.00 | 28.48 |
| ATOM | 4334 | CA | SER | A | 574 | 15.880 | 15.201 | −55.410 | 1.00 | 29.59 |
| ATOM | 4335 | CB | SER | A | 574 | 16.296 | 13.805 | −54.975 | 1.00 | 29.73 |
| ATOM | 4336 | OG | SER | A | 574 | 17.609 | 13.875 | −54.449 | 1.00 | 32.74 |
| ATOM | 4337 | C | SER | A | 574 | 14.526 | 15.134 | −56.095 | 1.00 | 29.70 |
| ATOM | 4338 | O | SER | A | 574 | 13.500 | 15.482 | −55.513 | 1.00 | 29.12 |
| ATOM | 4339 | N | ASP | A | 575 | 14.544 | 14.669 | −57.339 | 1.00 | 29.54 |
| ATOM | 4340 | CA | ASP | A | 575 | 13.337 | 14.435 | −58.109 | 1.00 | 29.98 |
| ATOM | 4341 | CB | ASP | A | 575 | 13.705 | 14.000 | −59.534 | 1.00 | 30.69 |
| ATOM | 4342 | CG | ASP | A | 575 | 14.324 | 15.125 | −60.331 | 1.00 | 33.96 |
| ATOM | 4343 | OD1 | ASP | A | 575 | 14.056 | 16.299 | −59.997 | 1.00 | 36.25 |
| ATOM | 4344 | OD2 | ASP | A | 575 | 15.083 | 14.846 | −61.290 | 1.00 | 37.94 |
| ATOM | 4345 | C | ASP | A | 575 | 12.519 | 13.358 | −57.428 | 1.00 | 28.93 |
| ATOM | 4346 | O | ASP | A | 575 | 13.050 | 12.633 | −56.600 | 1.00 | 28.34 |
| ATOM | 4347 | N | PRO | A | 576 | 11.217 | 13.267 | −57.760 | 1.00 | 28.58 |
| ATOM | 4348 | CA | PRO | A | 576 | 10.469 | 14.173 | −58.650 | 1.00 | 27.92 |
| ATOM | 4349 | CB | PRO | A | 576 | 9.319 | 13.294 | −59.131 | 1.00 | 28.52 |
| ATOM | 4350 | CG | PRO | A | 576 | 9.053 | 12.378 | −57.954 | 1.00 | 28.29 |
| ATOM | 4351 | CD | PRO | A | 576 | 10.377 | 12.159 | −57.267 | 1.00 | 28.76 |
| ATOM | 4352 | C | PRO | A | 576 | 9.894 | 15.397 | −57.938 | 1.00 | 27.25 |
| ATOM | 4353 | O | PRO | A | 576 | 9.887 | 15.452 | −56.703 | 1.00 | 28.13 |
| ATOM | 4354 | N | ASN | A | 577 | 9.394 | 16.360 | −58.707 | 1.00 | 25.62 |
| ATOM | 4355 | CA | ASN | A | 577 | 8.612 | 17.449 | −58.129 | 1.00 | 24.83 |
| ATOM | 4356 | CB | ASN | A | 577 | 8.013 | 18.336 | −59.224 | 1.00 | 24.90 |
| ATOM | 4357 | CG | ASN | A | 577 | 9.055 | 19.184 | −59.913 | 1.00 | 25.61 |
| ATOM | 4358 | OD1 | ASN | A | 577 | 10.176 | 19.321 | −59.423 | 1.00 | 25.83 |
| ATOM | 4359 | ND2 | ASN | A | 577 | 8.693 | 19.756 | −61.060 | 1.00 | 25.07 |
| ATOM | 4360 | C | ASN | A | 577 | 7.466 | 16.868 | −57.322 | 1.00 | 24.22 |
| ATOM | 4361 | O | ASN | A | 577 | 6.949 | 15.798 | −57.672 | 1.00 | 23.69 |
| ATOM | 4362 | N | HIS | A | 578 | 7.057 | 17.562 | −56.259 | 1.00 | 23.20 |
| ATOM | 4363 | CA | HIS | A | 578 | 5.830 | 17.179 | −55.570 | 1.00 | 22.96 |
| ATOM | 4364 | CB | HIS | A | 578 | 5.734 | 17.844 | −54.200 | 1.00 | 22.09 |
| ATOM | 4365 | CG | HIS | A | 578 | 6.874 | 17.538 | −53.285 | 1.00 | 21.80 |
| ATOM | 4366 | ND1 | HIS | A | 578 | 6.809 | 16.558 | −52.318 | 1.00 | 21.62 |
| ATOM | 4367 | CE1 | HIS | A | 578 | 7.948 | 16.530 | −51.645 | 1.00 | 21.36 |
| ATOM | 4368 | NE2 | HIS | A | 578 | 8.743 | 17.465 | −52.133 | 1.00 | 20.16 |
| ATOM | 4369 | CD2 | HIS | A | 578 | 8.096 | 18.109 | −53.160 | 1.00 | 19.78 |
| ATOM | 4370 | C | HIS | A | 578 | 4.697 | 17.707 | −56.429 | 1.00 | 23.58 |
| ATOM | 4371 | O | HIS | A | 578 | 4.814 | 18.794 | −56.976 | 1.00 | 23.64 |
| ATOM | 4372 | N | THR | A | 579 | 3.603 | 16.955 | −56.534 | 1.00 | 23.98 |
| ATOM | 4373 | CA | THR | A | 579 | 2.426 | 17.448 | −57.254 | 1.00 | 25.34 |
| ATOM | 4374 | CB | THR | A | 579 | 2.092 | 16.568 | −58.477 | 1.00 | 26.08 |
| ATOM | 4375 | OG1 | THR | A | 579 | 3.162 | 16.672 | −59.429 | 1.00 | 29.14 |
| ATOM | 4376 | CG2 | THR | A | 579 | 0.749 | 16.979 | −59.126 | 1.00 | 26.14 |
| ATOM | 4377 | C | THR | A | 579 | 1.259 | 17.480 | −56.291 | 1.00 | 25.05 |
| ATOM | 4378 | O | THR | A | 579 | 0.977 | 16.487 | −55.629 | 1.00 | 25.73 |
| ATOM | 4379 | N | TYR | A | 580 | 0.591 | 18.619 | −56.213 | 1.00 | 25.23 |
| ATOM | 4380 | CA | TYR | A | 580 | −0.450 | 18.802 | −55.211 | 1.00 | 25.68 |
| ATOM | 4381 | CB | TYR | A | 580 | 0.098 | 19.556 | −53.976 | 1.00 | 25.88 |
| ATOM | 4382 | CG | TYR | A | 580 | −0.931 | 19.763 | −52.866 | 1.00 | 26.21 |
| ATOM | 4383 | CD1 | TYR | A | 580 | −1.284 | 21.048 | −52.429 | 1.00 | 26.22 |
| ATOM | 4384 | CE1 | TYR | A | 580 | −2.256 | 21.233 | −51.399 | 1.00 | 27.53 |
| ATOM | 4385 | CZ | TYR | A | 580 | −2.860 | 20.111 | −50.841 | 1.00 | 27.08 |
| ATOM | 4386 | OH | TYR | A | 580 | −3.806 | 20.207 | −49.844 | 1.00 | 27.88 |
| ATOM | 4387 | CE2 | TYR | A | 580 | −2.510 | 18.841 | −51.264 | 1.00 | 26.97 |
| ATOM | 4388 | CD2 | TYR | A | 580 | −1.562 | 18.671 | −52.276 | 1.00 | 26.29 |
| ATOM | 4389 | C | TYR | A | 580 | −1.634 | 19.523 | −55.828 | 1.00 | 25.65 |
| ATOM | 4390 | O | TYR | A | 580 | −1.490 | 20.596 | −56.403 | 1.00 | 25.29 |
| ATOM | 4391 | N | THR | A | 581 | −2.813 | 18.915 | −55.732 | 1.00 | 25.95 |
| ATOM | 4392 | CA | THR | A | 581 | −4.015 | 19.629 | −56.117 | 1.00 | 25.78 |
| ATOM | 4393 | CB | THR | A | 581 | −5.016 | 18.700 | −56.806 | 1.00 | 26.75 |
| ATOM | 4394 | OG1 | THR | A | 581 | −4.332 | 18.000 | −57.855 | 1.00 | 26.57 |
| ATOM | 4395 | CG2 | THR | A | 581 | −6.189 | 19.498 | −57.397 | 1.00 | 27.62 |
| ATOM | 4396 | C | THR | A | 581 | −4.627 | 20.285 | −54.874 | 1.00 | 25.36 |
| ATOM | 4397 | O | THR | A | 581 | −5.024 | 19.595 | −53.935 | 1.00 | 25.22 |
| ATOM | 4398 | N | VAL | A | 582 | −4.685 | 21.615 | −54.880 | 1.00 | 24.54 |
| ATOM | 4399 | CA | VAL | A | 582 | −5.255 | 22.382 | −53.777 | 1.00 | 24.15 |
| ATOM | 4400 | CB | VAL | A | 582 | −5.006 | 23.915 | −53.953 | 1.00 | 24.21 |
| ATOM | 4401 | CG1 | VAL | A | 582 | −5.472 | 24.700 | −52.744 | 1.00 | 23.87 |
| ATOM | 4402 | CG2 | VAL | A | 582 | −3.514 | 24.218 | −54.219 | 1.00 | 25.33 |
| ATOM | 4403 | C | VAL | A | 582 | −6.759 | 22.063 | −53.706 | 1.00 | 24.32 |
| ATOM | 4404 | O | VAL | A | 582 | −7.478 | 22.204 | −54.700 | 1.00 | 23.26 |
| ATOM | 4405 | N | PRO | A | 583 | −7.236 | 21.587 | −52.546 | 1.00 | 24.10 |
| ATOM | 4406 | CA | PRO | A | 583 | −8.665 | 21.230 | −52.476 | 1.00 | 24.34 |
| ATOM | 4407 | CB | PRO | A | 583 | −8.865 | 20.763 | −51.022 | 1.00 | 24.33 |
| ATOM | 4408 | CG | PRO | A | 583 | −7.516 | 20.538 | −50.468 | 1.00 | 25.16 |
| ATOM | 4409 | CD | PRO | A | 583 | −6.508 | 21.310 | −51.294 | 1.00 | 24.72 |
| ATOM | 4410 | C | PRO | A | 583 | −9.597 | 22.404 | −52.768 | 1.00 | 24.50 |
| ATOM | 4411 | O | PRO | A | 583 | −9.262 | 23.558 | −52.487 | 1.00 | 23.93 |
| ATOM | 4412 | N | ALA | A | 584 | −10.756 | 22.104 | −53.350 | 1.00 | 24.61 |
| ATOM | 4413 | CA | ALA | A | 584 | −11.817 | 23.084 | −53.477 | 1.00 | 24.77 |
| ATOM | 4414 | CB | ALA | A | 584 | −12.065 | 23.439 | −54.943 | 1.00 | 25.29 |
| ATOM | 4415 | C | ALA | A | 584 | −13.036 | 22.434 | −52.847 | 1.00 | 24.94 |
| ATOM | 4416 | O | ALA | A | 584 | −13.922 | 21.932 | −53.537 | 1.00 | 25.03 |
| ATOM | 4417 | N | VAL | A | 585 | −13.052 | 22.406 | −51.517 | 1.00 | 24.24 |
| ATOM | 4418 | CA | VAL | A | 585 | −14.075 | 21.673 | −50.776 | 1.00 | 23.75 |
| ATOM | 4419 | CB | VAL | A | 585 | −13.465 | 20.452 | −50.029 | 1.00 | 24.50 |
| ATOM | 4420 | CG1 | VAL | A | 585 | −14.515 | 19.770 | −49.151 | 1.00 | 24.48 |
| ATOM | 4421 | CG2 | VAL | A | 585 | −12.863 | 19.447 | −51.026 | 1.00 | 25.32 |
| ATOM | 4422 | C | VAL | A | 585 | −14.707 | 22.639 | −49.781 | 1.00 | 23.20 |
| ATOM | 4423 | O | VAL | A | 585 | −13.999 | 23.347 | −49.065 | 1.00 | 22.13 |
| ATOM | 4424 | N | ALA | A | 586 | −16.044 | 22.679 | −49.739 | 1.00 | 22.43 |
| ATOM | 4425 | CA | ALA | A | 586 | −16.749 | 23.546 | −48.804 | 1.00 | 21.79 |
| ATOM | 4426 | CB | ALA | A | 586 | −18.240 | 23.212 | −48.820 | 1.00 | 22.19 |
| ATOM | 4427 | C | ALA | A | 586 | −16.160 | 23.324 | −47.389 | 1.00 | 21.57 |
| ATOM | 4428 | O | ALA | A | 586 | −15.954 | 22.180 | −46.990 | 1.00 | 20.89 |
| ATOM | 4429 | N | CYS | A | 587 | −15.872 | 24.414 | −46.679 | 1.00 | 21.59 |
| ATOM | 4430 | CA | CYS | A | 587 | −15.388 | 24.379 | −45.268 | 1.00 | 21.68 |
| ATOM | 4431 | CB | CYS | A | 587 | −16.131 | 23.323 | −44.441 | 1.00 | 22.08 |
| ATOM | 4432 | SG | CYS | A | 587 | −17.952 | 23.374 | −44.507 | 1.00 | 23.60 |
| ATOM | 4433 | C | CYS | A | 587 | −13.886 | 24.129 | −45.094 | 1.00 | 21.27 |
| ATOM | 4434 | O | CYS | A | 587 | −13.386 | 24.225 | −43.980 | 1.00 | 21.08 |
| ATOM | 4435 | N | VAL | A | 588 | −13.178 | 23.780 | −46.170 | 1.00 | 20.53 |
| ATOM | 4436 | CA | VAL | A | 588 | −11.742 | 23.499 | −46.085 | 1.00 | 20.52 |
| ATOM | 4437 | CB | VAL | A | 588 | −11.351 | 22.268 | −46.958 | 1.00 | 20.47 |
| ATOM | 4438 | CG1 | VAL | A | 588 | −9.846 | 21.959 | −46.844 | 1.00 | 20.87 |
| ATOM | 4439 | CG2 | VAL | A | 588 | −12.163 | 21.042 | −46.549 | 1.00 | 20.51 |
| ATOM | 4440 | C | VAL | A | 588 | −10.949 | 24.731 | −46.504 | 1.00 | 20.59 |
| ATOM | 4441 | O | VAL | A | 588 | −10.699 | 24.950 | −47.705 | 1.00 | 21.88 |
| ATOM | 4442 | N | THR | A | 589 | −10.533 | 25.522 | −45.528 | 1.00 | 19.56 |
| ATOM | 4443 | CA | THR | A | 589 | −9.903 | 26.807 | −45.795 | 1.00 | 19.48 |
| ATOM | 4444 | CB | THR | A | 589 | −10.595 | 27.914 | −44.988 | 1.00 | 20.13 |
| ATOM | 4445 | OG1 | THR | A | 589 | −10.527 | 27.565 | −43.592 | 1.00 | 21.49 |
| ATOM | 4446 | CG2 | THR | A | 589 | −12.085 | 28.018 | −45.410 | 1.00 | 20.03 |
| ATOM | 4447 | C | THR | A | 589 | −8.424 | 26.819 | −45.427 | 1.00 | 19.42 |
| ATOM | 4448 | O | THR | A | 589 | −7.694 | 27.767 | −45.743 | 1.00 | 18.66 |
| ATOM | 4449 | N | GLN | A | 590 | −7.995 | 25.772 | −44.734 | 1.00 | 19.62 |
| ATOM | 4450 | CA | GLN | A | 590 | −6.606 | 25.629 | −44.317 | 1.00 | 20.20 |
| ATOM | 4451 | CB | GLN | A | 590 | −6.359 | 26.261 | −42.939 | 1.00 | 21.06 |
| ATOM | 4452 | CG | GLN | A | 590 | −4.950 | 25.956 | −42.410 | 1.00 | 27.00 |
| ATOM | 4453 | CD | GLN | A | 590 | −4.184 | 27.189 | −41.989 | 1.00 | 33.74 |
| ATOM | 4454 | OE1 | GLN | A | 590 | −4.771 | 28.196 | −41.611 | 1.00 | 37.22 |
| ATOM | 4455 | NE2 | GLN | A | 590 | −2.855 | 27.118 | −42.066 | 1.00 | 36.77 |
| ATOM | 4456 | C | GLN | A | 590 | −6.247 | 24.159 | −44.295 | 1.00 | 19.00 |
| ATOM | 4457 | O | GLN | A | 590 | −7.004 | 23.335 | −43.771 | 1.00 | 18.70 |
| ATOM | 4458 | N | VAL | A | 591 | −5.113 | 23.811 | −44.904 | 1.00 | 17.70 |
| ATOM | 4459 | CA | VAL | A | 591 | −4.682 | 22.404 | −44.940 | 1.00 | 17.15 |
| ATOM | 4460 | CB | VAL | A | 591 | −4.843 | 21.750 | −46.330 | 1.00 | 17.82 |
| ATOM | 4461 | CG1 | VAL | A | 591 | −6.316 | 21.701 | −46.744 | 1.00 | 18.11 |
| ATOM | 4462 | CG2 | VAL | A | 591 | −3.970 | 22.470 | −47.390 | 1.00 | 17.60 |
| ATOM | 4463 | C | VAL | A | 591 | −3.213 | 22.360 | −44.551 | 1.00 | 17.42 |
| ATOM | 4464 | O | VAL | A | 591 | −2.531 | 23.377 | −44.638 | 1.00 | 17.32 |
| ATOM | 4465 | N | VAL | A | 592 | −2.731 | 21.206 | −44.090 | 1.00 | 17.26 |
| ATOM | 4466 | CA | VAL | A | 592 | −1.291 | 21.092 | −43.887 | 1.00 | 17.03 |
| ATOM | 4467 | CB | VAL | A | 592 | −0.762 | 21.198 | −42.365 | 1.00 | 18.70 |
| ATOM | 4468 | CG1 | VAL | A | 592 | 0.335 | 20.217 | −41.930 | 1.00 | 18.75 |
| ATOM | 4469 | CG2 | VAL | A | 592 | −1.810 | 21.731 | −41.315 | 1.00 | 15.36 |
| ATOM | 4470 | C | VAL | A | 592 | −0.736 | 19.951 | −44.730 | 1.00 | 16.99 |
| ATOM | 4471 | O | VAL | A | 592 | −1.318 | 18.862 | −44.828 | 1.00 | 16.23 |
| ATOM | 4472 | N | LYS | A | 593 | 0.357 | 20.253 | −45.403 | 1.00 | 15.38 |
| ATOM | 4473 | CA | LYS | A | 593 | 0.953 | 19.302 | −46.293 | 1.00 | 16.27 |
| ATOM | 4474 | CB | LYS | A | 593 | 1.301 | 20.010 | −47.616 | 1.00 | 16.69 |
| ATOM | 4475 | CG | LYS | A | 593 | 1.835 | 19.096 | −48.694 | 1.00 | 20.55 |
| ATOM | 4476 | CD | LYS | A | 593 | 0.791 | 18.101 | −49.203 | 1.00 | 24.73 |
| ATOM | 4477 | CE | LYS | A | 593 | 1.330 | 17.311 | −50.409 | 1.00 | 27.26 |
| ATOM | 4478 | NZ | LYS | A | 593 | 2.395 | 16.299 | −50.074 | 1.00 | 28.37 |
| ATOM | 4479 | C | LYS | A | 593 | 2.209 | 18.783 | −45.588 | 1.00 | 16.01 |
| ATOM | 4480 | O | LYS | A | 593 | 3.175 | 19.525 | −45.427 | 1.00 | 15.12 |
| ATOM | 4481 | N | GLU | A | 594 | 2.195 | 17.519 | −45.175 | 1.00 | 15.82 |
| ATOM | 4482 | CA | GLU | A | 594 | 3.308 | 16.969 | −44.407 | 1.00 | 16.23 |
| ATOM | 4483 | CB | GLU | A | 594 | 2.798 | 16.017 | −43.317 | 1.00 | 16.26 |
| ATOM | 4484 | CG | GLU | A | 594 | 1.866 | 16.732 | −42.299 | 1.00 | 16.62 |
| ATOM | 4485 | CD | GLU | A | 594 | 1.727 | 15.949 | −40.991 | 1.00 | 18.94 |
| ATOM | 4486 | OE1 | GLU | A | 594 | 1.267 | 14.778 | −41.024 | 1.00 | 21.31 |
| ATOM | 4487 | OE2 | GLU | A | 594 | 2.107 | 16.507 | −39.940 | 1.00 | 16.43 |
| ATOM | 4488 | C | GLU | A | 594 | 4.286 | 16.245 | −45.323 | 1.00 | 17.34 |
| ATOM | 4489 | O | GLU | A | 594 | 3.973 | 15.177 | −45.852 | 1.00 | 17.78 |
| ATOM | 4490 | N | ASP | A | 595 | 5.463 | 16.833 | −45.487 | 1.00 | 17.05 |
| ATOM | 4491 | CA | ASP | A | 595 | 6.481 | 16.326 | −46.405 | 1.00 | 17.21 |
| ATOM | 4492 | CB | ASP | A | 595 | 6.823 | 17.379 | −47.475 | 1.00 | 16.63 |
| ATOM | 4493 | CG | ASP | A | 595 | 5.678 | 17.619 | −48.455 | 1.00 | 17.91 |
| ATOM | 4494 | OD1 | ASP | A | 595 | 5.023 | 16.631 | −48.857 | 1.00 | 20.73 |
| ATOM | 4495 | OD2 | ASP | A | 595 | 5.434 | 18.795 | −48.844 | 1.00 | 18.08 |
| ATOM | 4496 | C | ASP | A | 595 | 7.734 | 15.955 | −45.631 | 1.00 | 17.47 |
| ATOM | 4497 | O | ASP | A | 595 | 7.915 | 16.375 | −44.492 | 1.00 | 16.44 |
| ATOM | 4498 | N | THR | A | 596 | 8.598 | 15.162 | −46.277 | 1.00 | 18.11 |
| ATOM | 4499 | CA | THR | A | 596 | 9.917 | 14.835 | −45.747 | 1.00 | 18.85 |
| ATOM | 4500 | CB | THR | A | 596 | 9.991 | 13.390 | −45.188 | 1.00 | 19.58 |
| ATOM | 4501 | OG1 | THR | A | 596 | 9.057 | 13.248 | −44.116 | 1.00 | 20.97 |
| ATOM | 4502 | CG2 | THR | A | 596 | 11.385 | 13.124 | −44.598 | 1.00 | 20.94 |
| ATOM | 4503 | C | THR | A | 596 | 10.895 | 14.978 | −46.914 | 1.00 | 19.34 |
| ATOM | 4504 | O | THR | A | 596 | 10.588 | 14.531 | −48.024 | 1.00 | 19.05 |
| ATOM | 4505 | N | TRP | A | 597 | 12.050 | 15.581 | −46.631 | 1.00 | 20.24 |
| ATOM | 4506 | CA | TRP | A | 597 | 13.074 | 15.921 | −47.633 | 1.00 | 21.94 |
| ATOM | 4507 | CB | TRP | A | 597 | 14.325 | 16.453 | −46.940 | 1.00 | 22.34 |
| ATOM | 4508 | CG | TRP | A | 597 | 15.445 | 16.854 | −47.882 | 1.00 | 23.75 |
| ATOM | 4509 | CD1 | TRP | A | 597 | 16.509 | 16.079 | −48.275 | 1.00 | 25.08 |
| ATOM | 4510 | NE1 | TRP | A | 597 | 17.327 | 16.801 | −49.138 | 1.00 | 24.85 |
| ATOM | 4511 | CE2 | TRP | A | 597 | 16.802 | 18.059 | −49.300 | 1.00 | 25.25 |
| ATOM | 4512 | CD2 | TRP | A | 597 | 15.611 | 18.128 | −48.527 | 1.00 | 24.09 |
| ATOM | 4513 | CE3 | TRP | A | 597 | 14.875 | 19.325 | −48.519 | 1.00 | 24.91 |
| ATOM | 4514 | CZ3 | TRP | A | 597 | 15.334 | 20.401 | −49.262 | 1.00 | 23.38 |
| ATOM | 4515 | CH2 | TRP | A | 597 | 16.520 | 20.299 | −50.028 | 1.00 | 25.14 |
| ATOM | 4516 | CZ2 | TRP | A | 597 | 17.265 | 19.137 | −50.053 | 1.00 | 22.49 |
| ATOM | 4517 | C | TRP | A | 597 | 13.424 | 14.708 | −48.473 | 1.00 | 23.42 |
| ATOM | 4518 | O | TRP | A | 597 | 13.675 | 13.635 | −47.939 | 1.00 | 22.37 |
| ATOM | 4519 | N | GLN | A | 598 | 13.409 | 14.904 | −49.788 | 1.00 | 25.26 |
| ATOM | 4520 | CA | GLN | A | 598 | 13.698 | 13.850 | −50.755 | 1.00 | 27.05 |
| ATOM | 4521 | CB | GLN | A | 598 | 12.936 | 14.124 | −52.052 | 1.00 | 26.51 |
| ATOM | 4522 | CG | GLN | A | 598 | 11.418 | 13.948 | −51.895 | 1.00 | 26.10 |
| ATOM | 4523 | CD | GLN | A | 598 | 10.642 | 14.209 | −53.156 | 1.00 | 26.76 |
| ATOM | 4524 | OE1 | GLN | A | 598 | 11.194 | 14.620 | −54.175 | 1.00 | 27.68 |
| ATOM | 4525 | NE2 | GLN | A | 598 | 9.340 | 13.990 | −53.095 | 1.00 | 25.96 |
| ATOM | 4526 | C | GLN | A | 598 | 15.204 | 13.787 | −50.977 | 1.00 | 29.21 |
| ATOM | 4527 | O | GLN | A | 598 | 15.794 | 14.694 | −51.574 | 1.00 | 28.61 |
| ATOM | 4528 | N | SER | A | 599 | 15.818 | 12.722 | −50.453 | 1.00 | 32.41 |
| ATOM | 4529 | CA | SER | A | 599 | 17.273 | 12.530 | −50.498 | 1.00 | 35.70 |
| ATOM | 4530 | CB | SER | A | 599 | 17.747 | 11.698 | −49.302 | 1.00 | 35.61 |
| ATOM | 4531 | OG | SER | A | 599 | 17.374 | 12.296 | −48.072 | 1.00 | 39.62 |
| ATOM | 4532 | C | SER | A | 599 | 17.703 | 11.831 | −51.785 | 1.00 | 36.66 |
| ATOM | 4533 | O | SER | A | 599 | 16.916 | 11.145 | −52.433 | 1.00 | 37.44 |
| ATOM | 4534 | OXT | SER | A | 599 | 18.863 | 11.922 | −52.194 | 1.00 | 38.18 |
| ATOM | 4535 | C1 | MAN | A | 601 | −3.602 | −3.018 | −46.412 | 1.00 | 102.64 |
| ATOM | 4536 | C2 | MAN | A | 601 | −4.584 | −2.109 | −47.156 | 1.00 | 102.73 |
| ATOM | 4537 | O2 | MAN | A | 601 | −3.951 | −1.548 | −48.288 | 1.00 | 102.91 |
| ATOM | 4538 | C3 | MAN | A | 601 | −5.867 | −2.845 | −47.570 | 1.00 | 102.38 |
| ATOM | 4539 | O3 | MAN | A | 601 | −6.544 | −2.112 | −48.566 | 1.00 | 102.32 |
| ATOM | 4540 | C4 | MAN | A | 601 | −5.640 | −4.269 | −48.082 | 1.00 | 102.18 |
| ATOM | 4541 | O4 | MAN | A | 601 | −6.860 | −4.967 | −47.984 | 1.00 | 101.76 |
| ATOM | 4542 | C5 | MAN | A | 601 | −4.561 | −5.018 | −47.298 | 1.00 | 102.40 |
| ATOM | 4543 | C6 | MAN | A | 601 | −4.172 | −6.307 | −48.010 | 1.00 | 102.48 |
| ATOM | 4544 | O6 | MAN | A | 601 | −3.156 | −6.957 | −47.280 | 1.00 | 102.80 |
| ATOM | 4545 | O5 | MAN | A | 601 | −3.400 | −4.222 | −47.131 | 1.00 | 102.71 |
| ATOM | 4546 | C1 | MAN | A | 602 | −29.428 | −4.974 | −42.477 | 1.00 | 77.32 |
| ATOM | 4547 | C2 | MAN | A | 602 | −28.973 | −6.434 | −42.405 | 1.00 | 77.44 |
| ATOM | 4548 | O2 | MAN | A | 602 | −30.120 | −7.253 | −42.347 | 1.00 | 77.72 |
| ATOM | 4549 | C3 | MAN | A | 602 | −28.044 | −6.835 | −43.565 | 1.00 | 77.20 |
| ATOM | 4550 | O3 | MAN | A | 602 | −27.940 | −8.239 | −43.664 | 1.00 | 76.96 |
| ATOM | 4551 | C4 | MAN | A | 602 | −28.487 | −6.260 | −44.909 | 1.00 | 77.22 |
| ATOM | 4552 | O4 | MAN | A | 602 | −27.471 | −6.474 | −45.862 | 1.00 | 76.90 |
| ATOM | 4553 | C5 | MAN | A | 602 | −28.766 | −4.766 | −44.768 | 1.00 | 77.47 |
| ATOM | 4554 | C6 | MAN | A | 602 | −29.185 | −4.115 | −46.081 | 1.00 | 77.84 |
| ATOM | 4555 | O6 | MAN | A | 602 | −28.163 | −3.228 | −46.483 | 1.00 | 78.24 |
| ATOM | 4556 | O5 | MAN | A | 602 | −29.768 | −4.562 | −43.790 | 1.00 | 77.46 |
| ATOM | 4557 | C1 | MAN | A | 603 | −18.689 | 25.235 | −53.677 | 1.00 | 47.04 |
| ATOM | 4558 | C2 | MAN | A | 603 | −20.074 | 24.872 | −53.114 | 1.00 | 51.09 |
| ATOM | 4559 | O2 | MAN | A | 603 | −21.044 | 25.065 | −54.120 | 1.00 | 52.42 |
| ATOM | 4560 | C3 | MAN | A | 603 | −20.141 | 23.420 | −52.620 | 1.00 | 51.78 |
| ATOM | 4561 | O3 | MAN | A | 603 | −21.465 | 23.079 | −52.262 | 1.00 | 53.36 |
| ATOM | 4562 | C4 | MAN | A | 603 | −19.602 | 22.466 | −53.686 | 1.00 | 51.72 |
| ATOM | 4563 | O4 | MAN | A | 603 | −19.615 | 21.142 | −53.209 | 1.00 | 51.94 |
| ATOM | 4564 | C5 | MAN | A | 603 | −18.179 | 22.911 | −54.021 | 1.00 | 51.66 |
| ATOM | 4565 | C6 | MAN | A | 603 | −17.421 | 21.906 | −54.892 | 1.00 | 53.80 |
| ATOM | 4566 | O6 | MAN | A | 603 | −17.915 | 21.885 | −56.214 | 1.00 | 55.49 |
| ATOM | 4567 | O5 | MAN | A | 603 | −18.217 | 24.223 | −54.581 | 1.00 | 49.62 |
| ATOM | 4568 | C1 | MAN | A | 605 | −4.678 | 15.117 | −57.896 | 1.00 | 58.79 |
| ATOM | 4569 | C2 | MAN | A | 605 | −3.360 | 15.555 | −58.538 | 1.00 | 58.65 |
| ATOM | 4570 | O2 | MAN | A | 605 | −2.564 | 14.412 | −58.722 | 1.00 | 59.38 |
| ATOM | 4571 | C3 | MAN | A | 605 | −3.570 | 16.269 | −59.878 | 1.00 | 58.70 |
| ATOM | 4572 | O3 | MAN | A | 605 | −2.523 | 15.985 | −60.778 | 1.00 | 59.27 |
| ATOM | 4573 | C4 | MAN | A | 605 | −4.915 | 15.892 | −60.491 | 1.00 | 58.73 |
| ATOM | 4574 | O4 | MAN | A | 605 | −5.084 | 16.538 | −61.730 | 1.00 | 59.62 |
| ATOM | 4575 | C5 | MAN | A | 605 | −6.054 | 16.284 | −59.547 | 1.00 | 58.90 |
| ATOM | 4576 | C6 | MAN | A | 605 | −7.370 | 15.612 | −59.932 | 1.00 | 58.75 |
| ATOM | 4577 | O6 | MAN | A | 605 | −7.255 | 14.219 | −59.738 | 1.00 | 59.42 |
| ATOM | 4578 | O5 | MAN | A | 605 | −5.730 | 16.034 | −58.173 | 1.00 | 58.30 |
| ATOM | 4579 | C1 | MAN | A | 606 | −10.273 | 28.688 | −42.727 | 1.00 | 25.90 |
| ATOM | 4580 | C2 | MAN | A | 606 | −9.839 | 27.944 | −41.452 | 1.00 | 28.64 |
| ATOM | 4581 | O2 | MAN | A | 606 | −9.245 | 28.909 | −40.620 | 1.00 | 28.84 |
| ATOM | 4582 | C3 | MAN | A | 606 | −10.999 | 27.249 | −40.710 | 1.00 | 29.65 |
| ATOM | 4583 | O3 | MAN | A | 606 | −10.568 | 26.763 | −39.441 | 1.00 | 28.85 |
| ATOM | 4584 | C4 | MAN | A | 606 | −12.203 | 28.177 | −40.551 | 1.00 | 30.36 |
| ATOM | 4585 | O4 | MAN | A | 606 | −13.330 | 27.463 | −40.084 | 1.00 | 30.29 |
| ATOM | 4586 | C5 | MAN | A | 606 | −12.553 | 28.769 | −41.914 | 1.00 | 30.72 |
| ATOM | 4587 | C6 | MAN | A | 606 | −13.730 | 29.731 | −41.778 | 1.00 | 33.97 |
| ATOM | 4588 | O6 | MAN | A | 606 | −13.624 | 30.732 | −42.762 | 1.00 | 36.82 |
| ATOM | 4589 | O5 | MAN | A | 606 | −11.434 | 29.464 | −42.435 | 1.00 | 28.12 |
| ATOM | 4590 | C1 | MAN | A | 607 | −31.396 | 1.963 | −40.521 | 1.00 | 50.29 |
| ATOM | 4591 | C2 | MAN | A | 607 | −30.220 | 1.790 | −41.485 | 1.00 | 52.65 |
| ATOM | 4592 | O2 | MAN | A | 607 | −30.541 | 0.785 | −42.419 | 1.00 | 54.93 |
| ATOM | 4593 | C3 | MAN | A | 607 | −29.845 | 3.092 | −42.208 | 1.00 | 52.48 |
| ATOM | 4594 | O3 | MAN | A | 607 | −28.932 | 2.836 | −43.251 | 1.00 | 53.01 |
| ATOM | 4595 | C4 | MAN | A | 607 | −31.068 | 3.818 | −42.766 | 1.00 | 52.78 |
| ATOM | 4596 | O4 | MAN | A | 607 | −30.672 | 5.070 | −43.297 | 1.00 | 52.92 |
| ATOM | 4597 | C5 | MAN | A | 607 | −32.103 | 3.985 | −41.652 | 1.00 | 52.23 |
| ATOM | 4598 | C6 | MAN | A | 607 | −33.331 | 4.749 | −42.153 | 1.00 | 52.96 |
| ATOM | 4599 | O6 | MAN | A | 607 | −34.520 | 4.076 | −41.791 | 1.00 | 52.95 |
| ATOM | 4600 | O5 | MAN | A | 607 | −32.451 | 2.702 | −41.127 | 1.00 | 51.79 |
| ATOM | 4601 | C1 | MAN | A | 608 | 3.870 | 15.416 | −59.489 | 1.00 | 37.21 |
| ATOM | 4602 | C2 | MAN | A | 608 | 5.134 | 15.938 | −60.168 | 1.00 | 40.45 |
| ATOM | 4603 | O2 | MAN | A | 608 | 6.091 | 14.903 | −60.120 | 1.00 | 38.47 |
| ATOM | 4604 | C3 | MAN | A | 608 | 4.872 | 16.381 | −61.608 | 1.00 | 42.66 |
| ATOM | 4605 | O3 | MAN | A | 608 | 6.071 | 16.726 | −62.263 | 1.00 | 44.20 |
| ATOM | 4606 | C4 | MAN | A | 608 | 4.122 | 15.321 | −62.401 | 1.00 | 44.80 |
| ATOM | 4607 | O4 | MAN | A | 608 | 3.708 | 15.907 | −63.612 | 1.00 | 47.73 |
| ATOM | 4608 | C5 | MAN | A | 608 | 2.893 | 14.887 | −61.597 | 1.00 | 44.80 |
| ATOM | 4609 | C6 | MAN | A | 608 | 2.042 | 13.861 | −62.342 | 1.00 | 47.55 |
| ATOM | 4610 | O6 | MAN | A | 608 | 1.085 | 14.582 | −63.104 | 1.00 | 49.87 |
| ATOM | 4611 | O5 | MAN | A | 608 | 3.262 | 14.423 | −60.302 | 1.00 | 42.18 |
| ATOM | 4612 | C1 | NAG | A | 611 | 3.450 | −2.354 | −8.282 | 1.00 | 23.44 |
| ATOM | 4613 | C2 | NAG | A | 611 | 3.474 | −0.875 | −7.878 | 1.00 | 24.51 |
| ATOM | 4614 | N2 | NAG | A | 611 | 4.425 | −0.077 | −8.630 | 1.00 | 21.95 |
| ATOM | 4615 | C7 | NAG | A | 611 | 4.123 | 0.454 | −9.818 | 1.00 | 22.94 |
| ATOM | 4616 | O7 | NAG | A | 611 | 3.030 | 0.322 | −10.367 | 1.00 | 20.93 |
| ATOM | 4617 | C8 | NAG | A | 611 | 5.216 | 1.232 | −10.481 | 1.00 | 21.54 |
| ATOM | 4618 | C3 | NAG | A | 611 | 3.741 | −0.713 | −6.380 | 1.00 | 25.60 |
| ATOM | 4619 | O3 | NAG | A | 611 | 3.676 | 0.655 | −6.047 | 1.00 | 24.91 |
| ATOM | 4620 | C4 | NAG | A | 611 | 2.741 | −1.528 | −5.554 | 1.00 | 25.70 |
| ATOM | 4621 | O4 | NAG | A | 611 | 3.196 | −1.598 | −4.227 | 1.00 | 28.27 |
| ATOM | 4622 | C5 | NAG | A | 611 | 2.648 | −2.952 | −6.086 | 1.00 | 26.18 |
| ATOM | 4623 | C6 | NAG | A | 611 | 1.524 | −3.738 | −5.397 | 1.00 | 26.64 |
| ATOM | 4624 | O6 | NAG | A | 611 | 0.278 | −3.081 | −5.497 | 1.00 | 25.38 |
| ATOM | 4625 | O5 | NAG | A | 611 | 2.437 | −2.975 | −7.488 | 1.00 | 24.34 |
| ATOM | 4626 | C1 | NAG | A | 612 | 2.499 | −0.713 | −3.326 | 1.00 | 32.04 |
| ATOM | 4627 | C2 | NAG | A | 612 | 2.710 | −1.192 | −1.879 | 1.00 | 35.81 |
| ATOM | 4628 | N2 | NAG | A | 612 | 2.254 | −2.556 | −1.666 | 1.00 | 37.89 |
| ATOM | 4629 | C7 | NAG | A | 612 | 3.072 | −3.605 | −1.753 | 1.00 | 39.19 |
| ATOM | 4630 | O7 | NAG | A | 612 | 4.277 | −3.517 | −2.031 | 1.00 | 40.58 |
| ATOM | 4631 | C8 | NAG | A | 612 | 2.439 | −4.947 | −1.507 | 1.00 | 38.98 |
| ATOM | 4632 | C3 | NAG | A | 612 | 2.012 | −0.256 | −0.899 | 1.00 | 37.96 |
| ATOM | 4633 | O3 | NAG | A | 612 | 2.352 | −0.666 | 0.403 | 1.00 | 41.23 |
| ATOM | 4634 | C4 | NAG | A | 612 | 2.491 | 1.176 | −1.129 | 1.00 | 37.63 |
| ATOM | 4635 | O4 | NAG | A | 612 | 1.789 | 2.053 | −0.278 | 1.00 | 40.85 |
| ATOM | 4636 | C5 | NAG | A | 612 | 2.294 | 1.565 | −2.604 | 1.00 | 35.10 |
| ATOM | 4637 | C6 | NAG | A | 612 | 2.785 | 2.982 | −2.903 | 1.00 | 31.93 |
| ATOM | 4638 | O6 | NAG | A | 612 | 4.188 | 2.994 | −3.008 | 0.58 | 32.70 |
| ATOM | 4639 | O5 | NAG | A | 612 | 2.974 | 0.625 | −3.425 | 1.00 | 31.95 |
| ATOM | 4640 | O8 | BTB | A | 620 | −1.213 | 18.638 | −21.639 | 1.00 | 23.78 |
| ATOM | 4641 | C8 | BTB | A | 620 | −1.255 | 19.440 | −22.838 | 1.00 | 17.50 |
| ATOM | 4642 | C7 | BTB | A | 620 | −2.257 | 18.851 | −23.831 | 1.00 | 15.39 |
| ATOM | 4643 | N | BTB | A | 620 | −1.808 | 17.505 | −24.294 | 1.00 | 13.88 |
| ATOM | 4644 | C5 | BTB | A | 620 | −1.274 | 17.600 | −25.684 | 1.00 | 12.99 |
| ATOM | 4645 | C6 | BTB | A | 620 | 0.017 | 18.399 | −25.786 | 1.00 | 14.67 |
| ATOM | 4646 | O6 | BTB | A | 620 | 0.949 | 18.004 | −24.768 | 1.00 | 16.93 |
| ATOM | 4647 | C2 | BTB | A | 620 | −2.926 | 16.495 | −24.191 | 1.00 | 13.33 |
| ATOM | 4648 | C4 | BTB | A | 620 | −4.238 | 16.972 | −24.835 | 1.00 | 13.45 |
| ATOM | 4649 | O4 | BTB | A | 620 | −4.167 | 17.018 | −26.265 | 1.00 | 14.77 |
| ATOM | 4650 | C3 | BTB | A | 620 | −3.213 | 16.295 | −22.703 | 1.00 | 13.18 |
| ATOM | 4651 | O3 | BTB | A | 620 | −1.984 | 15.920 | −22.059 | 1.00 | 12.74 |
| ATOM | 4652 | C1 | BTB | A | 620 | −2.501 | 15.161 | −24.845 | 1.00 | 13.57 |
| ATOM | 4653 | O1 | BTB | A | 620 | −3.463 | 14.138 | −24.525 | 1.00 | 13.07 |
| ATOM | 4654 | O | WAT | W | 1 | −7.741 | 16.530 | −28.587 | 1.00 | 12.90 |
| ATOM | 4655 | O | WAT | W | 2 | −1.955 | 18.721 | −7.814 | 1.00 | 11.77 |
| ATOM | 4656 | O | WAT | W | 3 | −17.101 | 16.033 | −19.836 | 1.00 | 15.26 |
| ATOM | 4657 | O | WAT | W | 4 | −1.389 | 7.464 | −24.070 | 1.00 | 15.86 |
| ATOM | 4658 | O | WAT | W | 5 | −8.070 | 20.758 | −43.462 | 1.00 | 19.56 |
| ATOM | 4659 | O | WAT | W | 6 | −12.959 | 28.534 | −26.860 | 1.00 | 16.12 |
| ATOM | 4660 | O | WAT | W | 7 | −0.502 | 31.488 | −57.004 | 1.00 | 33.06 |
| ATOM | 4661 | O | WAT | W | 8 | 2.095 | 5.710 | −17.808 | 1.00 | 18.68 |
| ATOM | 4662 | O | WAT | W | 9 | −7.601 | 14.567 | −6.827 | 1.00 | 14.97 |
| ATOM | 4663 | O | WAT | W | 10 | 24.863 | 23.325 | −37.431 | 1.00 | 32.31 |
| ATOM | 4664 | O | WAT | W | 11 | −22.569 | 7.289 | −10.357 | 1.00 | 17.52 |
| ATOM | 4665 | O | WAT | W | 12 | −18.987 | 1.758 | −22.078 | 1.00 | 23.03 |
| ATOM | 4666 | O | WAT | W | 13 | −3.226 | 16.264 | −54.338 | 1.00 | 32.98 |
| ATOM | 4667 | O | WAT | W | 14 | 6.141 | 16.546 | −42.196 | 1.00 | 16.00 |
| ATOM | 4668 | O | WAT | W | 15 | −10.356 | 21.827 | −22.675 | 1.00 | 13.52 |
| ATOM | 4669 | O | WAT | W | 16 | −3.130 | 25.355 | −17.925 | 1.00 | 14.01 |
| ATOM | 4670 | O | WAT | W | 17 | −11.823 | 29.479 | −29.411 | 1.00 | 17.50 |
| ATOM | 4671 | O | WAT | W | 18 | −14.383 | 15.964 | −19.553 | 1.00 | 13.02 |
| ATOM | 4672 | O | WAT | W | 19 | −1.180 | 16.935 | −10.101 | 1.00 | 18.86 |
| ATOM | 4673 | O | WAT | W | 20 | −31.133 | 23.501 | 4.462 | 1.00 | 16.66 |
| ATOM | 4674 | O | WAT | W | 21 | −4.819 | 24.193 | −15.023 | 1.00 | 14.31 |
| ATOM | 4675 | O | WAT | W | 22 | 1.709 | 22.276 | −4.126 | 1.00 | 21.96 |
| ATOM | 4676 | O | WAT | W | 23 | −5.339 | 21.386 | −7.463 | 1.00 | 15.78 |
| ATOM | 4677 | O | WAT | W | 24 | −17.232 | 15.476 | 1.374 | 1.00 | 17.64 |
| ATOM | 4678 | O | WAT | W | 25 | −11.449 | 4.860 | −24.929 | 1.00 | 17.45 |
| ATOM | 4679 | O | WAT | W | 26 | −17.555 | 17.679 | −39.815 | 1.00 | 23.23 |
| ATOM | 4680 | O | WAT | W | 27 | 10.075 | 17.015 | −49.295 | 1.00 | 24.05 |
| ATOM | 4681 | O | WAT | W | 28 | −16.018 | −0.740 | −24.205 | 1.00 | 18.07 |
| ATOM | 4682 | O | WAT | W | 29 | 9.446 | 24.991 | −37.612 | 1.00 | 19.20 |
| ATOM | 4683 | O | WAT | W | 30 | −4.165 | 26.137 | −12.642 | 1.00 | 18.33 |
| ATOM | 4684 | O | WAT | W | 31 | 2.771 | 22.947 | −14.916 | 1.00 | 25.80 |
| ATOM | 4685 | O | WAT | W | 32 | −12.297 | 21.394 | −35.680 | 1.00 | 14.89 |
| ATOM | 4686 | O | WAT | W | 33 | −24.061 | 13.570 | 10.081 | 1.00 | 24.96 |
| ATOM | 4687 | O | WAT | W | 34 | 10.032 | 29.725 | −56.684 | 1.00 | 26.97 |
| ATOM | 4688 | O | WAT | W | 35 | 0.231 | 4.133 | −28.595 | 1.00 | 17.67 |
| ATOM | 4689 | O | WAT | W | 36 | 0.335 | 2.173 | −30.650 | 1.00 | 18.32 |
| ATOM | 4690 | O | WAT | W | 37 | −10.199 | 24.315 | −42.717 | 1.00 | 22.38 |
| ATOM | 4691 | O | WAT | W | 38 | −14.151 | 12.872 | −8.204 | 1.00 | 16.16 |
| ATOM | 4692 | O | WAT | W | 39 | −2.710 | 9.564 | −16.092 | 1.00 | 14.10 |
| ATOM | 4693 | O | WAT | W | 40 | 5.954 | 7.990 | −32.401 | 1.00 | 16.59 |
| ATOM | 4694 | O | WAT | W | 41 | 0.294 | 5.561 | −25.249 | 1.00 | 17.87 |
| ATOM | 4695 | O | WAT | W | 42 | 2.102 | 15.148 | −37.718 | 1.00 | 14.64 |
| ATOM | 4696 | O | WAT | W | 43 | −19.351 | 1.384 | −26.295 | 1.00 | 20.27 |
| ATOM | 4697 | O | WAT | W | 44 | −19.623 | 9.533 | −17.751 | 1.00 | 14.67 |
| ATOM | 4698 | O | WAT | W | 45 | 3.117 | 18.767 | −36.336 | 1.00 | 12.66 |
| ATOM | 4699 | O | WAT | W | 46 | −15.016 | 16.950 | 0.662 | 1.00 | 20.14 |
| ATOM | 4700 | O | WAT | W | 47 | −22.261 | 4.600 | −10.993 | 1.00 | 16.38 |
| ATOM | 4701 | O | WAT | W | 48 | −12.926 | 5.474 | −22.680 | 1.00 | 19.85 |
| ATOM | 4702 | O | WAT | W | 49 | 5.564 | 17.071 | −37.018 | 1.00 | 16.82 |
| ATOM | 4703 | O | WAT | W | 50 | −19.848 | 20.552 | −2.718 | 1.00 | 19.38 |
| ATOM | 4704 | O | WAT | W | 51 | −15.859 | 17.744 | −41.901 | 1.00 | 19.69 |
| ATOM | 4705 | O | WAT | W | 52 | −16.430 | 25.522 | −1.123 | 1.00 | 19.97 |
| ATOM | 4706 | O | WAT | W | 53 | −15.978 | 5.366 | −12.193 | 1.00 | 26.02 |
| ATOM | 4707 | O | WAT | W | 54 | −1.637 | 9.365 | −26.035 | 1.00 | 14.42 |
| ATOM | 4708 | O | WAT | W | 55 | −10.759 | 27.212 | −30.898 | 1.00 | 17.28 |
| ATOM | 4709 | O | WAT | W | 56 | −11.509 | 0.756 | −13.101 | 1.00 | 20.70 |
| ATOM | 4710 | O | WAT | W | 57 | −16.950 | 15.108 | 4.727 | 1.00 | 23.86 |
| ATOM | 4711 | O | WAT | W | 58 | −25.368 | 26.009 | −7.106 | 1.00 | 25.08 |
| ATOM | 4712 | O | WAT | W | 59 | −16.870 | 22.937 | −3.651 | 1.00 | 17.56 |
| ATOM | 4713 | O | WAT | W | 60 | −14.388 | 13.258 | −40.897 | 1.00 | 27.90 |
| ATOM | 4714 | O | WAT | W | 61 | −1.509 | −4.779 | −6.723 | 1.00 | 31.01 |
| ATOM | 4715 | O | WAT | W | 62 | −1.973 | 27.723 | −11.521 | 1.00 | 21.99 |
| ATOM | 4716 | O | WAT | W | 63 | −1.159 | −10.623 | −29.592 | 1.00 | 36.68 |
| ATOM | 4717 | O | WAT | W | 64 | −1.943 | 16.930 | −42.957 | 1.00 | 21.57 |
| ATOM | 4718 | O | WAT | W | 65 | −1.507 | 25.238 | −40.032 | 1.00 | 31.36 |
| ATOM | 4719 | O | WAT | W | 66 | −4.023 | 5.499 | −31.787 | 1.00 | 20.03 |
| ATOM | 4720 | O | WAT | W | 67 | −13.383 | 13.873 | −21.065 | 1.00 | 12.01 |
| ATOM | 4721 | O | WAT | W | 68 | −15.098 | 10.726 | −24.467 | 1.00 | 24.34 |
| ATOM | 4722 | O | WAT | W | 69 | −2.122 | 13.975 | −13.435 | 1.00 | 12.51 |
| ATOM | 4723 | O | WAT | W | 70 | −4.807 | 19.360 | −43.270 | 1.00 | 20.93 |
| ATOM | 4724 | O | WAT | W | 71 | −26.028 | 26.143 | −33.768 | 1.00 | 28.78 |
| ATOM | 4725 | O | WAT | W | 72 | −19.347 | 21.638 | 3.482 | 1.00 | 19.92 |
| ATOM | 4726 | O | WAT | W | 73 | −27.299 | 24.219 | −6.045 | 1.00 | 20.97 |
| ATOM | 4727 | O | WAT | W | 74 | −21.114 | −0.343 | −28.050 | 1.00 | 22.38 |
| ATOM | 4728 | O | WAT | W | 75 | −5.818 | 34.483 | −11.645 | 1.00 | 20.61 |
| ATOM | 4729 | O | WAT | W | 76 | 6.048 | 1.098 | −23.393 | 1.00 | 16.77 |
| ATOM | 4730 | O | WAT | W | 77 | −3.946 | 23.711 | −39.552 | 1.00 | 25.07 |
| ATOM | 4731 | O | WAT | W | 78 | −18.572 | 21.631 | −41.884 | 1.00 | 25.98 |
| ATOM | 4732 | O | WAT | W | 79 | 5.239 | 26.273 | −31.646 | 1.00 | 27.95 |
| ATOM | 4733 | O | WAT | W | 80 | 0.054 | 15.597 | −45.905 | 1.00 | 28.45 |
| ATOM | 4734 | O | WAT | W | 81 | −3.130 | 21.534 | −5.652 | 1.00 | 20.95 |
| ATOM | 4735 | O | WAT | W | 82 | −12.534 | 4.331 | −20.095 | 1.00 | 17.49 |
| ATOM | 4736 | O | WAT | W | 83 | 0.785 | 16.541 | −14.558 | 1.00 | 14.65 |
| ATOM | 4737 | O | WAT | W | 84 | −5.197 | 12.827 | −31.553 | 1.00 | 14.10 |
| ATOM | 4738 | O | WAT | W | 85 | −16.738 | 26.994 | −34.463 | 1.00 | 23.74 |
| ATOM | 4739 | O | WAT | W | 86 | 3.596 | 22.076 | −36.828 | 1.00 | 22.68 |
| ATOM | 4740 | O | WAT | W | 87 | 5.170 | 14.460 | −40.572 | 1.00 | 26.56 |
| ATOM | 4741 | O | WAT | W | 88 | −12.322 | 21.050 | 0.328 | 1.00 | 30.36 |
| ATOM | 4742 | O | WAT | W | 89 | 7.426 | 14.327 | −48.857 | 1.00 | 26.44 |
| ATOM | 4743 | O | WAT | W | 90 | −13.702 | 19.025 | 1.863 | 1.00 | 28.28 |
| ATOM | 4744 | O | WAT | W | 91 | 8.794 | 2.010 | −23.444 | 1.00 | 34.69 |
| ATOM | 4745 | O | WAT | W | 92 | −6.185 | 5.529 | −30.210 | 1.00 | 17.03 |
| ATOM | 4746 | O | WAT | W | 93 | −18.081 | 20.709 | −4.839 | 1.00 | 17.61 |
| ATOM | 4747 | O | WAT | W | 94 | −15.469 | 13.082 | −22.717 | 1.00 | 15.55 |
| ATOM | 4748 | O | WAT | W | 95 | 13.101 | 16.811 | −29.771 | 1.00 | 29.16 |
| ATOM | 4749 | O | WAT | W | 96 | −25.944 | 7.031 | −2.628 | 1.00 | 27.42 |
| ATOM | 4750 | O | WAT | W | 97 | −4.552 | 34.207 | −7.388 | 1.00 | 22.19 |
| ATOM | 4751 | O | WAT | W | 98 | −2.231 | −9.858 | −32.291 | 1.00 | 26.28 |
| ATOM | 4752 | O | WAT | W | 99 | 5.314 | 10.271 | −28.762 | 1.00 | 29.39 |
| ATOM | 4753 | O | WAT | W | 100 | −15.379 | 27.478 | −46.620 | 1.00 | 37.77 |
| ATOM | 4754 | O | WAT | W | 101 | 26.815 | 24.874 | −36.295 | 1.00 | 31.11 |
| ATOM | 4755 | O | WAT | W | 102 | −18.489 | −0.112 | −24.256 | 1.00 | 23.63 |
| ATOM | 4756 | O | WAT | W | 103 | −23.763 | 26.890 | −9.454 | 1.00 | 20.50 |
| ATOM | 4757 | O | WAT | W | 104 | −10.933 | 23.904 | −50.315 | 1.00 | 25.63 |
| ATOM | 4758 | O | WAT | W | 105 | 5.864 | 12.071 | −41.668 | 1.00 | 29.27 |
| ATOM | 4759 | O | WAT | W | 106 | 2.526 | 9.409 | −13.116 | 1.00 | 20.75 |
| ATOM | 4760 | O | WAT | W | 107 | −11.557 | −6.653 | −10.981 | 1.00 | 30.68 |
| ATOM | 4761 | O | WAT | W | 108 | −14.882 | 7.238 | −22.254 | 1.00 | 24.81 |
| ATOM | 4762 | O | WAT | W | 109 | −5.331 | −13.390 | −25.293 | 1.00 | 35.63 |
| ATOM | 4763 | O | WAT | W | 110 | −8.068 | 24.248 | −40.534 | 1.00 | 39.14 |
| ATOM | 4764 | O | WAT | W | 111 | −0.779 | 14.419 | −43.060 | 1.00 | 24.01 |
| ATOM | 4765 | O | WAT | W | 112 | −22.279 | 12.054 | −26.750 | 1.00 | 32.13 |
| ATOM | 4766 | O | WAT | W | 113 | −26.829 | 1.352 | −33.787 | 1.00 | 26.97 |
| ATOM | 4767 | O | WAT | W | 114 | −14.120 | 14.116 | 3.214 | 1.00 | 36.89 |
| ATOM | 4768 | O | WAT | W | 115 | 0.582 | −9.914 | −21.103 | 1.00 | 23.30 |
| ATOM | 4769 | O | WAT | W | 116 | −24.305 | 22.723 | 6.995 | 1.00 | 21.67 |
| ATOM | 4770 | O | WAT | W | 117 | −28.275 | 12.468 | −15.419 | 1.00 | 21.87 |
| ATOM | 4771 | O | WAT | W | 118 | 3.699 | 27.669 | −20.781 | 1.00 | 34.08 |
| ATOM | 4772 | O | WAT | W | 119 | −30.428 | 26.452 | 3.757 | 1.00 | 24.51 |
| ATOM | 4773 | O | WAT | W | 120 | 19.168 | 26.858 | −59.022 | 1.00 | 33.45 |
| ATOM | 4774 | O | WAT | W | 121 | −8.803 | 21.729 | −1.693 | 1.00 | 18.30 |
| ATOM | 4775 | O | WAT | W | 122 | 2.863 | 1.621 | −31.755 | 1.00 | 19.96 |
| ATOM | 4776 | O | WAT | W | 123 | −2.357 | 28.930 | −56.725 | 1.00 | 35.51 |
| ATOM | 4777 | O | WAT | W | 124 | −16.780 | 5.504 | −21.523 | 1.00 | 31.34 |
| ATOM | 4778 | O | WAT | W | 125 | 6.216 | 18.141 | −30.592 | 1.00 | 20.87 |
| ATOM | 4779 | O | WAT | W | 126 | 11.789 | 32.722 | −38.773 | 1.00 | 40.41 |
| ATOM | 4780 | O | WAT | W | 127 | −5.001 | 7.195 | −45.656 | 1.00 | 35.03 |
| ATOM | 4781 | O | WAT | W | 128 | −18.743 | 1.608 | −1.861 | 1.00 | 32.49 |
| ATOM | 4782 | O | WAT | W | 129 | −25.089 | −1.945 | −20.935 | 1.00 | 35.17 |
| ATOM | 4783 | O | WAT | W | 130 | −7.097 | −2.177 | −28.928 | 1.00 | 30.93 |
| ATOM | 4784 | O | WAT | W | 131 | −12.591 | 2.907 | −11.929 | 1.00 | 18.60 |
| ATOM | 4785 | O | WAT | W | 132 | −17.913 | −2.374 | −39.429 | 1.00 | 29.36 |
| ATOM | 4786 | O | WAT | W | 133 | −6.507 | −7.038 | −37.710 | 1.00 | 37.27 |
| ATOM | 4787 | O | WAT | W | 134 | −0.628 | 7.596 | −18.660 | 1.00 | 20.01 |
| ATOM | 4788 | O | WAT | W | 135 | −11.683 | 28.527 | −37.016 | 1.00 | 36.45 |
| ATOM | 4789 | O | WAT | W | 136 | −3.169 | 33.267 | −18.049 | 1.00 | 24.89 |
| ATOM | 4790 | O | WAT | W | 137 | −16.742 | 8.938 | −23.161 | 1.00 | 26.79 |
| ATOM | 4791 | O | WAT | W | 138 | −28.456 | 17.726 | −22.449 | 1.00 | 32.61 |
| ATOM | 4792 | O | WAT | W | 139 | 25.559 | 27.237 | −45.392 | 1.00 | 43.21 |
| ATOM | 4793 | O | WAT | W | 140 | −26.925 | 5.789 | −41.722 | 1.00 | 26.97 |
| ATOM | 4794 | O | WAT | W | 141 | −16.907 | 20.013 | −43.283 | 1.00 | 29.68 |
| ATOM | 4795 | O | WAT | W | 142 | −20.029 | 5.119 | −1.799 | 1.00 | 24.76 |
| ATOM | 4796 | O | WAT | W | 143 | 8.706 | 1.050 | −13.115 | 1.00 | 32.81 |
| ATOM | 4797 | O | WAT | W | 144 | −4.353 | 22.506 | −1.252 | 1.00 | 24.86 |
| ATOM | 4798 | O | WAT | W | 145 | −29.660 | 14.750 | −15.295 | 1.00 | 32.62 |
| ATOM | 4799 | O | WAT | W | 146 | 2.173 | 2.240 | −12.124 | 1.00 | 26.38 |
| ATOM | 4800 | O | WAT | W | 147 | 4.174 | −4.659 | −14.794 | 1.00 | 25.20 |
| ATOM | 4801 | O | WAT | W | 148 | −10.913 | 29.083 | −33.130 | 1.00 | 27.78 |
| ATOM | 4802 | O | WAT | W | 149 | −21.448 | 30.157 | −10.670 | 1.00 | 25.07 |
| ATOM | 4803 | O | WAT | W | 150 | −23.296 | 18.641 | −36.646 | 1.00 | 27.11 |
| ATOM | 4804 | O | WAT | W | 151 | −19.426 | 8.262 | −24.240 | 1.00 | 25.11 |
| ATOM | 4805 | O | WAT | W | 152 | 4.729 | −0.512 | −31.679 | 1.00 | 23.50 |
| ATOM | 4806 | O | WAT | W | 153 | 9.247 | 19.703 | −33.306 | 1.00 | 23.44 |
| ATOM | 4807 | O | WAT | W | 154 | 6.024 | 15.401 | −22.768 | 1.00 | 27.11 |
| ATOM | 4808 | O | WAT | W | 155 | −16.077 | 30.180 | −4.530 | 1.00 | 23.52 |
| ATOM | 4809 | O | WAT | W | 156 | −0.038 | 14.751 | −8.812 | 1.00 | 25.64 |
| ATOM | 4810 | O | WAT | W | 157 | 2.962 | 18.631 | −29.190 | 1.00 | 18.13 |
| ATOM | 4811 | O | WAT | W | 158 | 8.793 | 12.371 | −36.745 | 1.00 | 23.77 |
| ATOM | 4812 | O | WAT | W | 159 | −22.406 | 9.468 | −0.415 | 1.00 | 21.66 |
| ATOM | 4813 | O | WAT | W | 160 | −10.961 | 33.685 | −7.076 | 1.00 | 25.30 |
| ATOM | 4814 | O | WAT | W | 161 | −8.504 | 27.891 | −3.964 | 1.00 | 33.88 |
| ATOM | 4815 | O | WAT | W | 162 | 6.836 | 20.663 | −32.439 | 1.00 | 24.97 |
| ATOM | 4816 | O | WAT | W | 163 | 4.292 | 23.232 | −29.206 | 1.00 | 32.74 |
| ATOM | 4817 | O | WAT | W | 164 | 2.350 | 3.656 | −15.645 | 1.00 | 23.29 |
| ATOM | 4818 | O | WAT | W | 165 | −17.377 | 10.190 | −20.605 | 1.00 | 25.21 |
| ATOM | 4819 | O | WAT | W | 166 | −23.426 | 24.714 | 4.551 | 1.00 | 26.12 |
| ATOM | 4820 | O | WAT | W | 167 | 0.338 | 1.730 | −14.995 | 1.00 | 31.29 |
| ATOM | 4821 | O | WAT | W | 168 | −3.303 | 17.836 | −46.350 | 1.00 | 28.34 |
| ATOM | 4822 | O | WAT | W | 169 | 1.465 | 6.514 | −14.840 | 1.00 | 22.81 |
| ATOM | 4823 | O | WAT | W | 170 | 2.409 | 11.466 | −4.481 | 1.00 | 29.82 |
| ATOM | 4824 | O | WAT | W | 171 | 0.998 | 19.313 | −20.348 | 1.00 | 31.57 |
| ATOM | 4825 | O | WAT | W | 172 | 7.556 | −3.076 | −34.213 | 1.00 | 31.62 |
| ATOM | 4826 | O | WAT | W | 173 | −25.163 | 1.132 | −18.852 | 1.00 | 33.45 |
| ATOM | 4827 | O | WAT | W | 174 | −25.606 | 17.471 | −26.509 | 1.00 | 27.89 |
| ATOM | 4828 | O | WAT | W | 175 | 5.952 | 32.621 | −65.955 | 1.00 | 42.20 |
| ATOM | 4829 | O | WAT | W | 176 | −27.397 | 26.421 | −12.489 | 1.00 | 29.06 |
| ATOM | 4830 | O | WAT | W | 177 | −17.506 | 35.918 | −29.284 | 1.00 | 36.40 |
| ATOM | 4831 | O | WAT | W | 178 | −18.298 | 7.055 | −19.628 | 1.00 | 30.04 |
| ATOM | 4832 | O | WAT | W | 179 | −24.383 | 14.811 | −26.605 | 1.00 | 29.51 |
| ATOM | 4833 | O | WAT | W | 180 | −1.204 | 27.462 | −35.328 | 1.00 | 29.93 |
| ATOM | 4834 | O | WAT | W | 181 | −14.112 | 33.822 | −23.916 | 1.00 | 34.66 |
| ATOM | 4835 | O | WAT | W | 182 | 2.887 | 26.714 | −9.619 | 1.00 | 34.18 |
| ATOM | 4836 | O | WAT | W | 183 | −16.062 | 4.698 | 1.046 | 1.00 | 32.44 |
| ATOM | 4837 | O | WAT | W | 184 | −13.340 | 36.111 | −4.359 | 1.00 | 39.14 |
| ATOM | 4838 | O | WAT | W | 185 | 9.661 | 34.457 | −47.977 | 1.00 | 37.66 |
| ATOM | 4839 | O | WAT | W | 186 | −8.465 | 24.284 | −1.237 | 1.00 | 33.71 |
| ATOM | 4840 | O | WAT | W | 187 | 16.971 | 15.520 | −43.951 | 1.00 | 42.49 |
| ATOM | 4841 | O | WAT | W | 188 | −12.038 | −14.614 | −20.299 | 1.00 | 34.37 |
| ATOM | 4842 | O | WAT | W | 189 | −5.887 | 22.387 | −40.784 | 1.00 | 33.70 |
| ATOM | 4843 | O | WAT | W | 190 | −3.962 | −18.100 | −17.720 | 1.00 | 31.33 |
| ATOM | 4844 | O | WAT | W | 191 | −30.888 | 11.643 | −15.288 | 1.00 | 36.84 |
| ATOM | 4845 | O | WAT | W | 192 | 11.576 | 13.142 | −37.752 | 1.00 | 32.89 |
| ATOM | 4846 | O | WAT | W | 193 | −7.856 | 3.348 | −41.927 | 1.00 | 34.02 |
| ATOM | 4847 | O | WAT | W | 194 | −20.849 | 7.518 | 7.652 | 1.00 | 32.37 |
| ATOM | 4848 | O | WAT | W | 195 | 16.954 | 13.938 | −58.514 | 1.00 | 42.65 |
| ATOM | 4849 | O | WAT | W | 196 | −31.884 | 7.593 | −13.893 | 1.00 | 37.54 |
| ATOM | 4850 | O | WAT | W | 197 | 4.560 | −14.190 | −17.137 | 1.00 | 36.09 |
| ATOM | 4851 | O | WAT | W | 198 | 1.116 | 27.617 | −39.051 | 1.00 | 37.08 |
| ATOM | 4852 | O | WAT | W | 199 | −1.019 | −12.134 | −21.800 | 1.00 | 36.12 |
| ATOM | 4853 | O | WAT | W | 200 | 8.350 | 0.111 | −21.198 | 1.00 | 36.56 |
| ATOM | 4854 | O | WAT | W | 201 | −2.691 | 31.235 | −26.910 | 1.00 | 32.08 |
| ATOM | 4855 | O | WAT | W | 202 | 13.222 | 30.530 | −38.626 | 1.00 | 36.46 |
| ATOM | 4856 | O | WAT | W | 203 | −11.218 | 19.535 | −54.549 | 1.00 | 35.12 |
| ATOM | 4857 | O | WAT | W | 204 | −5.623 | 10.865 | −46.910 | 1.00 | 35.48 |
| ATOM | 4858 | O | WAT | W | 205 | −18.073 | 1.743 | −43.946 | 1.00 | 40.62 |
| ATOM | 4859 | O | WAT | W | 206 | −32.195 | 23.231 | 2.102 | 1.00 | 34.73 |
| ATOM | 4860 | O | WAT | W | 207 | −24.204 | 8.994 | −2.941 | 1.00 | 30.29 |
| ATOM | 4861 | O | WAT | W | 208 | −4.771 | 18.292 | −48.610 | 1.00 | 31.87 |
| ATOM | 4862 | O | WAT | W | 209 | −17.156 | 23.843 | −40.674 | 1.00 | 35.73 |
| ATOM | 4863 | O | WAT | W | 210 | 8.319 | 13.422 | −13.297 | 1.00 | 37.43 |
| ATOM | 4864 | O | WAT | W | 211 | −25.962 | 8.559 | −33.791 | 1.00 | 33.46 |
| ATOM | 4865 | O | WAT | W | 212 | −36.129 | 8.276 | 3.147 | 1.00 | 40.24 |
| ATOM | 4866 | O | WAT | W | 213 | 20.833 | 21.074 | −56.185 | 1.00 | 39.11 |
| ATOM | 4867 | O | WAT | W | 214 | −17.726 | 14.087 | 8.330 | 1.00 | 39.10 |
| ATOM | 4868 | O | WAT | W | 215 | 8.944 | 8.011 | −10.493 | 1.00 | 41.24 |
| ATOM | 4869 | O | WAT | W | 216 | −16.566 | 35.858 | −11.282 | 1.00 | 38.90 |
| ATOM | 4870 | O | WAT | W | 217 | −20.560 | 11.198 | −43.128 | 1.00 | 34.83 |
| ATOM | 4871 | O | WAT | W | 218 | 3.261 | −0.833 | −39.177 | 1.00 | 32.67 |
| ATOM | 4872 | O | WAT | W | 219 | −22.370 | −13.152 | −34.412 | 1.00 | 59.42 |
| ATOM | 4873 | O | WAT | W | 220 | −24.775 | 6.925 | 5.968 | 1.00 | 34.28 |
| ATOM | 4874 | O | WAT | W | 221 | −20.357 | 21.098 | −45.702 | 1.00 | 36.83 |
| ATOM | 4875 | O | WAT | W | 222 | 2.502 | 28.932 | −40.686 | 1.00 | 36.85 |
| ATOM | 4876 | O | WAT | W | 223 | −17.630 | −5.533 | −21.334 | 1.00 | 35.08 |
| ATOM | 4877 | O | WAT | W | 224 | −19.358 | −1.912 | −43.190 | 1.00 | 35.83 |
| ATOM | 4878 | O | WAT | W | 225 | −14.632 | 25.995 | −42.094 | 1.00 | 41.12 |
| ATOM | 4879 | O | WAT | W | 226 | −28.967 | 5.606 | −22.103 | 1.00 | 45.63 |
| ATOM | 4880 | O | WAT | W | 227 | −4.326 | 9.934 | 5.097 | 1.00 | 44.46 |
| ATOM | 4881 | O | WAT | W | 228 | 3.983 | 22.711 | −17.336 | 1.00 | 46.21 |
| ATOM | 4882 | O | WAT | W | 229 | −17.238 | 16.931 | 6.949 | 1.00 | 42.44 |
| ATOM | 4883 | O | WAT | W | 230 | −25.871 | 18.809 | 10.364 | 1.00 | 36.15 |
| ATOM | 4884 | O | WAT | W | 231 | −23.524 | 31.294 | −19.082 | 1.00 | 35.99 |
| ATOM | 4885 | O | WAT | W | 232 | −5.261 | −9.321 | −38.277 | 1.00 | 43.42 |
| ATOM | 4886 | O | WAT | W | 233 | −22.757 | 28.188 | −29.173 | 1.00 | 39.56 |
| ATOM | 4887 | O | WAT | W | 234 | −25.699 | 26.238 | −0.299 | 1.00 | 40.26 |
| ATOM | 4888 | O | WAT | W | 235 | −21.884 | −4.007 | −22.294 | 1.00 | 38.01 |
| ATOM | 4889 | O | WAT | W | 236 | −6.696 | −17.346 | −27.753 | 1.00 | 39.83 |
| ATOM | 4890 | O | WAT | W | 237 | −18.052 | 5.515 | 3.888 | 1.00 | 37.82 |
| ATOM | 4891 | O | WAT | W | 238 | −6.073 | 35.708 | −9.057 | 1.00 | 36.25 |
| ATOM | 4892 | O | WAT | W | 239 | −8.876 | 2.984 | −44.368 | 1.00 | 45.85 |
| ATOM | 4893 | O | WAT | W | 240 | 9.232 | 31.613 | −65.496 | 1.00 | 45.82 |
| ATOM | 4894 | O | WAT | W | 241 | −28.246 | 26.806 | −0.118 | 1.00 | 36.45 |
| ATOM | 4895 | O | WAT | W | 242 | −27.793 | 14.675 | −45.312 | 1.00 | 52.10 |
| ATOM | 4896 | O | WAT | W | 243 | 7.463 | 14.079 | −55.045 | 1.00 | 36.97 |
| ATOM | 4897 | O | WAT | W | 244 | −28.572 | 4.769 | −1.130 | 1.00 | 36.20 |
| ATOM | 4898 | O | WAT | W | 245 | 8.221 | 12.936 | −50.870 | 1.00 | 38.81 |
| ATOM | 4899 | O | WAT | W | 246 | −23.302 | −2.082 | −27.191 | 1.00 | 32.98 |
| ATOM | 4900 | O | WAT | W | 247 | −13.035 | 8.248 | −46.620 | 1.00 | 51.73 |
| ATOM | 4901 | O | WAT | W | 248 | −11.869 | 31.852 | −50.157 | 1.00 | 56.13 |
| ATOM | 4902 | O | WAT | W | 249 | 0.898 | 13.920 | −6.521 | 1.00 | 27.38 |
| ATOM | 4903 | O | WAT | W | 250 | 20.427 | 30.852 | −45.446 | 1.00 | 35.54 |
| ATOM | 4904 | O | WAT | W | 251 | −1.397 | 12.400 | −44.617 | 1.00 | 39.33 |
| ATOM | 4905 | O | WAT | W | 252 | −27.354 | 24.696 | −3.162 | 1.00 | 35.38 |
| ATOM | 4906 | O | WAT | W | 253 | 17.587 | 20.557 | −31.069 | 1.00 | 41.51 |
| ATOM | 4907 | O | WAT | W | 254 | −7.936 | 35.055 | −7.354 | 1.00 | 39.02 |
| ATOM | 4908 | O | WAT | W | 255 | −22.469 | 7.215 | −2.044 | 1.00 | 38.25 |
| ATOM | 4909 | O | WAT | W | 256 | 2.038 | 15.474 | −52.963 | 1.00 | 50.04 |
| ATOM | 4910 | O | WAT | W | 257 | 10.889 | 10.184 | −21.700 | 1.00 | 44.84 |
| ATOM | 4911 | O | WAT | W | 258 | −11.714 | 10.583 | 4.136 | 1.00 | 42.70 |
| ATOM | 4912 | O | WAT | W | 259 | −14.719 | 6.574 | 2.959 | 1.00 | 43.18 |
| ATOM | 4913 | O | WAT | W | 260 | −16.694 | 25.390 | −37.688 | 1.00 | 36.77 |
| ATOM | 4914 | O | WAT | W | 261 | −9.212 | 13.388 | −48.363 | 1.00 | 38.05 |
| ATOM | 4915 | O | WAT | W | 264 | −0.611 | −1.965 | −3.253 | 1.00 | 37.95 |
| ATOM | 4916 | O | WAT | W | 265 | −16.380 | 30.998 | −14.262 | 1.00 | 32.44 |
| ATOM | 4917 | O | WAT | W | 266 | 9.420 | 16.012 | −61.368 | 1.00 | 35.22 |
| ATOM | 4918 | O | WAT | W | 267 | −4.976 | −15.180 | −21.223 | 1.00 | 45.50 |
| ATOM | 4919 | O | WAT | W | 268 | −16.631 | 33.287 | −14.201 | 1.00 | 34.50 |
| ATOM | 4920 | O | WAT | W | 269 | −16.883 | 34.052 | −32.249 | 1.00 | 36.88 |
| ATOM | 4921 | O | WAT | W | 270 | −8.293 | −16.006 | −14.535 | 1.00 | 34.80 |
| ATOM | 4922 | O | WAT | W | 273 | 0.240 | 4.589 | −13.868 | 1.00 | 32.34 |
| ATOM | 4923 | O | WAT | W | 275 | 3.657 | 14.447 | −55.516 | 1.00 | 43.54 |
| ATOM | 4924 | O | WAT | W | 276 | −17.602 | 20.784 | −51.471 | 1.00 | 38.88 |
| ATOM | 4925 | O | WAT | W | 277 | −10.479 | 31.683 | −30.513 | 1.00 | 40.35 |
| ATOM | 4926 | O | WAT | W | 278 | −10.974 | 4.308 | −5.745 | 1.00 | 40.30 |
| ATOM | 4927 | O | WAT | W | 280 | −4.336 | 36.908 | −17.666 | 1.00 | 34.01 |
| ATOM | 4928 | O | WAT | W | 281 | 6.720 | 33.970 | −53.572 | 1.00 | 38.81 |
| ATOM | 4929 | O | WAT | W | 282 | −30.457 | 23.527 | −0.621 | 1.00 | 34.61 |
| ATOM | 4930 | O | WAT | W | 283 | 16.969 | 17.394 | −30.816 | 1.00 | 55.74 |
| ATOM | 4931 | O | WAT | W | 284 | −24.391 | 5.834 | −24.909 | 1.00 | 37.92 |
| ATOM | 4932 | O | WAT | W | 285 | 4.567 | 9.814 | −4.438 | 1.00 | 44.83 |
| ATOM | 4933 | O | WAT | W | 286 | −24.370 | −7.328 | −27.875 | 1.00 | 56.18 |
| ATOM | 4934 | O | WAT | W | 287 | −21.605 | 11.887 | 9.715 | 1.00 | 43.15 |
| ATOM | 4935 | O | WAT | W | 288 | 8.603 | 0.412 | −37.887 | 1.00 | 40.47 |
| ATOM | 4936 | O | WAT | W | 290 | −20.056 | 21.495 | 6.073 | 1.00 | 41.68 |
| ATOM | 4937 | O | WAT | W | 291 | −3.221 | 28.158 | −33.448 | 1.00 | 44.24 |
| ATOM | 4938 | O | WAT | W | 292 | 9.171 | 9.103 | −38.735 | 1.00 | 34.30 |
| ATOM | 4939 | O | WAT | W | 293 | 2.894 | 22.763 | −25.829 | 1.00 | 38.57 |
| ATOM | 4940 | O | WAT | W | 294 | −29.901 | 19.604 | −14.929 | 1.00 | 37.66 |
| ATOM | 4941 | O | WAT | W | 296 | −4.579 | 30.229 | −29.110 | 1.00 | 40.57 |
| ATOM | 4942 | O | WAT | W | 297 | −23.821 | 11.441 | −33.187 | 1.00 | 42.81 |
| ATOM | 4943 | O | WAT | W | 298 | −26.753 | −3.087 | −31.243 | 1.00 | 39.08 |
| ATOM | 4944 | O | WAT | W | 300 | −10.820 | 35.024 | −53.050 | 1.00 | 55.39 |
| ATOM | 4945 | O | WAT | W | 302 | −1.992 | 7.169 | −31.692 | 1.00 | 39.12 |
| ATOM | 4946 | O | WAT | W | 303 | −15.282 | −19.000 | −23.770 | 1.00 | 34.62 |
| ATOM | 4947 | O | WAT | W | 304 | 12.106 | 10.568 | −25.112 | 1.00 | 39.04 |
| ATOM | 4948 | O | WAT | W | 305 | 2.585 | 2.766 | 1.880 | 1.00 | 53.88 |
| ATOM | 4949 | O | WAT | W | 306 | 3.680 | 21.122 | −19.818 | 1.00 | 46.35 |
| ATOM | 4950 | O | WAT | W | 307 | 22.759 | 24.721 | −48.099 | 1.00 | 40.35 |
| ATOM | 4951 | O | WAT | W | 309 | −17.062 | −6.726 | −19.202 | 1.00 | 41.54 |
| ATOM | 4952 | O | WAT | W | 311 | 12.594 | 1.109 | −31.461 | 1.00 | 47.85 |
| ATOM | 4953 | O | WAT | W | 312 | 23.347 | 25.060 | −50.638 | 1.00 | 49.64 |
| ATOM | 4954 | O | WAT | W | 314 | −18.291 | 4.422 | −19.151 | 1.00 | 39.17 |
| ATOM | 4955 | O | WAT | W | 315 | −11.815 | −7.807 | −8.676 | 1.00 | 37.74 |
| ATOM | 4956 | O | WAT | W | 316 | −25.147 | 1.885 | −4.649 | 1.00 | 44.72 |
| ATOM | 4957 | O | WAT | W | 317 | −36.473 | 13.592 | 5.315 | 1.00 | 44.38 |
| ATOM | 4958 | O | WAT | W | 318 | −17.587 | 20.023 | −46.231 | 1.00 | 48.30 |
| ATOM | 4959 | O | WAT | W | 319 | −16.081 | 29.024 | −54.668 | 1.00 | 39.40 |
| ATOM | 4960 | O | WAT | W | 320 | −14.210 | 32.143 | −5.494 | 1.00 | 42.73 |
| ATOM | 4961 | O | WAT | W | 321 | −15.274 | 28.830 | −38.916 | 1.00 | 46.47 |
| ATOM | 4962 | O | WAT | W | 322 | −32.792 | 22.221 | −3.433 | 1.00 | 41.52 |
| ATOM | 4963 | O | WAT | W | 323 | −32.475 | 16.905 | −12.401 | 1.00 | 46.29 |
| ATOM | 4964 | O | WAT | W | 325 | 15.341 | 22.212 | −60.490 | 1.00 | 34.67 |
| ATOM | 4965 | O | WAT | W | 326 | −12.668 | 8.518 | −41.723 | 1.00 | 36.26 |
| ATOM | 4966 | O | WAT | W | 327 | 4.709 | 20.490 | −10.568 | 1.00 | 38.04 |
| ATOM | 4967 | O | WAT | W | 328 | 13.937 | 10.625 | −29.312 | 1.00 | 38.32 |
| ATOM | 4968 | O | WAT | W | 329 | −21.964 | 9.615 | −24.896 | 1.00 | 40.43 |
| ATOM | 4969 | O | WAT | W | 330 | 19.325 | 25.925 | −40.199 | 1.00 | 51.36 |
| ATOM | 4970 | O | WAT | W | 331 | −19.010 | 8.073 | −45.255 | 1.00 | 46.06 |
| ATOM | 4971 | O | WAT | W | 332 | −25.024 | −2.892 | −29.306 | 1.00 | 41.74 |
| ATOM | 4972 | O | WAT | W | 333 | −16.593 | −7.067 | −23.297 | 1.00 | 38.54 |
| ATOM | 4973 | O | WAT | W | 334 | −17.517 | 24.078 | 2.157 | 1.00 | 45.82 |
| ATOM | 4974 | O | WAT | W | 335 | −19.123 | 31.941 | 0.010 | 1.00 | 38.91 |
| ATOM | 4975 | O | WAT | W | 337 | 10.677 | 21.901 | −62.740 | 1.00 | 44.31 |
| ATOM | 4976 | O | WAT | W | 338 | 4.510 | 15.230 | −51.810 | 1.00 | 42.12 |
| ATOM | 4977 | O | WAT | W | 339 | 13.979 | 14.161 | −43.380 | 1.00 | 46.42 |
| ATOM | 4978 | O | WAT | W | 341 | 5.979 | −11.625 | −28.739 | 1.00 | 43.09 |
| ATOM | 4979 | O | WAT | W | 342 | −19.453 | 13.347 | 10.394 | 1.00 | 42.44 |
| ATOM | 4980 | O | WAT | W | 343 | 7.085 | 23.050 | −30.796 | 1.00 | 34.94 |
| ATOM | 4981 | O | WAT | W | 345 | 6.471 | 24.087 | −63.943 | 1.00 | 45.16 |
| ATOM | 4982 | O | WAT | W | 347 | 3.734 | 22.842 | −12.031 | 1.00 | 43.85 |
| ATOM | 4983 | O | WAT | W | 348 | −17.739 | 7.564 | 5.723 | 1.00 | 43.77 |
| ATOM | 4984 | O | WAT | W | 351 | −22.014 | 31.372 | −24.708 | 1.00 | 42.69 |
| ATOM | 4985 | O | WAT | W | 352 | 25.016 | 25.103 | −46.967 | 1.00 | 40.51 |
| ATOM | 4986 | O | WAT | W | 353 | 7.969 | 32.464 | −67.637 | 1.00 | 57.78 |
| ATOM | 4987 | O | WAT | W | 354 | −27.444 | 5.101 | 5.861 | 1.00 | 48.31 |
| ATOM | 4988 | O | WAT | W | 356 | 8.012 | 11.087 | −40.867 | 1.00 | 47.51 |
| ATOM | 4989 | O | WAT | W | 357 | 4.974 | 29.116 | −17.433 | 1.00 | 43.72 |
| ATOM | 4990 | O | WAT | W | 358 | −0.457 | 9.488 | −45.288 | 1.00 | 45.63 |
| ATOM | 4991 | O | WAT | W | 360 | −3.090 | 36.536 | −12.138 | 1.00 | 46.29 |
| ATOM | 4992 | O | WAT | W | 361 | 20.072 | 19.772 | −36.896 | 1.00 | 38.73 |
| ATOM | 4993 | O | WAT | W | 363 | −26.217 | 15.345 | −28.735 | 1.00 | 49.07 |
| ATOM | 4994 | O | WAT | W | 365 | −25.308 | 0.100 | −48.602 | 1.00 | 60.01 |
| ATOM | 4995 | O | WAT | W | 367 | 19.369 | 29.586 | −58.438 | 1.00 | 48.19 |
| ATOM | 4996 | O | WAT | W | 369 | 12.808 | 11.144 | −54.427 | 1.00 | 48.48 |
| ATOM | 4997 | O | WAT | W | 370 | 9.410 | 2.674 | −16.115 | 1.00 | 44.59 |
| ATOM | 4998 | O | WAT | W | 372 | −10.249 | 38.564 | −13.215 | 1.00 | 48.60 |
| ATOM | 4999 | O | WAT | W | 373 | −24.151 | 16.211 | 10.617 | 1.00 | 42.16 |
| ATOM | 5000 | O | WAT | W | 375 | −6.459 | 31.697 | −48.106 | 1.00 | 46.03 |
| ATOM | 5001 | O | WAT | W | 376 | −11.605 | 27.116 | −1.562 | 1.00 | 44.58 |
| ATOM | 5002 | O | WAT | W | 377 | −4.703 | 24.150 | −62.673 | 1.00 | 48.70 |
| ATOM | 5003 | O | WAT | W | 379 | 6.889 | 0.036 | −7.530 | 1.00 | 45.35 |
| ATOM | 5004 | O | WAT | W | 381 | −13.601 | 32.742 | −32.002 | 1.00 | 51.37 |
| ATOM | 5005 | O | WAT | W | 383 | −28.077 | 5.243 | −4.688 | 1.00 | 37.46 |

### Example 10

### Homology between TrGA and AaGA

The crystal structure of the TrGA identified in Example 9 was superposed on the previously identified crystal structure of the Aspergillus awamori GA (AaGA). The AaGA crystal structure was obtained from the protein database (PDB) and the form of AaGA that was crystallized was the form containing only a catalytic domain (PDB entry number: 1GLM). The structure of the TrGA with all three regions intact was determined to 1.8 Angstrom resolution herein (see Table 9 and Example 9). Using the coordinates (see Table 9), the structure was aligned with the coordinates of the catalytic domain from Aspergillus awamori strain X100 that was determined previously (Aleshin et al., J. Mol. Biol. 238: 575-591 (1994)). As seen in FIGS. 6-7, the structure of the catalytic domain overlapped very closely and allowed the identification of equivalent residues based on the structural superposition.

Based on this analysis, sites were identified that could be mutated in TrGA and result in increased thermostability and/or specific activity. There sites include 108, 124, 175, and 316 at the active site. Also identified were specific pairwise variants Y47W/Y315F and Y47F/Y315W. Other sites identified were I43, D44, P45, D46, R122, R125, V181, E242, Y310, D313, V314, N317, R408, and N409. Because of the high structural homology, it is expected that beneficial variants found at sites in the TrGA would have similar consequence in Aspergillus awamori and other homologous glucoamylases.

The TrGA linker, residues 454-490 is defined as the segment spanning the region between two disulfide bridges, one between residues 222 and 453 and one between residues 491 and 587. Nine of the residues in the linker are prolines. From the crystal structure, the linker extends from the back of the molecule in a wide arc followed by an abrupt turn after the lysine 477 residue on the surface near the substrate binding surface. The linker extends as a random coil that is anchored by interactions of the side chains of Tyr 452, Pro 465, Phe 470, Gln 474, Pro 475, Lys 477, Val 480 and Tyr 486 to regions on the surface of the catalytic domain.

The starch binding domain is composed of a beta-sandwich of two twisted beta sheets, tethered at one end by a disulfide bridge between Cys 491 and Cys 587 and at the other end, having a series of loops that comprise a binding site for starch connected by long loops. The structure of the TrGA SBD is quite similar to the averaged structure of the AnGA SBD determined by NMR (Sorimachi et al., Structure 5: 647-661 (1997)) and the SBD of beta amylase from Bacillus cereus (Mikami, B. et al., Biochemistry 38: 7050-61 (1999)). FIG. 9 shows an alignment of the AnGA and TrGA crystal structures including the SBD. When aligned with one or both of these SBD's, one loop stands out as being highly variable, corresponding to residues 537-543 (in A. niger the loop is 554-560 and in B. cereus the loop is 462-465). In the NMR structure of beta-cyclodextrin, a starch analog complexed to the SBD of AnGA (Sorimachi et al. (1997) supra), the loop shifts substantially upon binding to cyclodextrin. Thus, this loop is designated the “flexible loop.” This flexible loop forms part of the “binding site 2” (see FIG. 9 for this binding site in TrGA). A second binding site was also identified in AnGA (binding site 1), a primary site that shares similarities with other carbohydrate binding proteins. Overall, conservation of residues and even side conformations in the binding site 1 of these SBDs is very high. The figures demonstrate the interactions in these binding sites between the SBD and the catalytic domain that serve to bind to the starch.

Taken together, there appears to be a common pattern for the interactions between the linker and SBD with the catalytic domain. The interaction is in the form of an anchoring side chain that interacts with the surface area of the neighboring domain. In general, the anchor residue is found on the linker segment. In the case of interactions between the CD and SBD, the anchor residues can be contributed from either domain as in the case of residues Ile 43 and Phe 29 that come from the CD or residue 592, which comes from the SBD.

### Example 11

### Model of Acarbose Binding to TrGA

The crystal structure of the TrGA complexed with the inhibitor acarbose has been determined. Crystals of the complex were obtained by soaking pre-grown native TrGA crystals in acarbose. After soaking for 3 days the crystals were mounted in a seal glass capillary tube and x-ray diffraction was collected with a Rigaku Raxis IV++ image plate detector to a resolution of 2.0 Å. The coordinates were fitted to a difference electron density map. The model was refined to an R-factor of 0.154 with an R-free of 0.201 for a total of 41276 reflection representing all data collected between 27 and 2.0 Å resolution. The model of the resulting refined structure is shown in FIG. 9.

Based on the knowledge that the presence of the SBD has an impact on hydrolysis of insoluble starch, it followed that there should be an interaction of the SBD with larger starch molecules. Thus, the structure of the TrGA was compared with known structures of (1) an acarbose bound CD of AaGA and (2) an SBD from A. niger complexed with beta-cyclodextrin. This showed that the beta-cyclodextrin bound at binding site 2 was close to the substrate location as indicated by the location of acarbose bound to the A. awamori CD. Thus, the coordinates of acarbose from the structure model of the AaGA (pdb entylGAl, Aleshin, et al. 1994 supra) were aligned into the TrGA active site. Further, the AnGA SBD structure bound to cyclodextrin (pdb entry TACO: Sorimachi, et al 1997 supra) was aligned. From this, a model was made for acarbose binding to TrGA (see FIG. 9). The model showed that the SBD would localize the TrGA CD near disrupted starch, and also prevent the enzyme from diffusing away from the substrate while releasing the product from the active site after hydrolysis. The SBD of TrGA would bind to starch along site 1, and favor localization where a disrupted fragment could bind to site 2 within a loose end that points into the catalytic site (the active side for the catalytic domain). This model shows how the proposed function of the enzyme is contributed by the structure of the SBD and linker. The amino acid side chains involved in the specific interaction between the CD, the linker and the SBD are specific for Trichoderma reesei GA, however, in other glucoamylases, complementary sequence changes would enable similar overall interactions and domain juxtaposition.

Based on this model, sites were identified for which substitutions could be made in the TrGA SBD to result in increased stability and/or specific activity. Thus, two loops that are part of binding site 1 are likely candidates for alterations to increase or decrease binding to the larger starch molecule. These are loop 1 (aa 560-570) and loop 2 (aa 523-527). Because the two Trp (tryptophan) residues at amino acids 525 and 572 are likely involved directly in starch binding, they would not be as conducive to change. However, the underlying residues, including 516-518 would be conducive, as would the underlying residues 558-562. The loop from residues 570-578 is also a good candidate for alterations. Residues 534-541 are part of the binding site 2 that interacts with the catalytic site on the CD. Thus, these might be a good candidate for alterations that may increase or decrease specific activity.

Because of the high structural homology of the TrGA SBD, it is expected that beneficial variants found at sites in Trichoderma reesei GA would have similar consequences in Aspergillus awamori and other homologous glucoamylases. Thus, the structure of the TrGA SBD provides a basis for engineering this and related enzymes for altered properties as compared to a parent glucoamylase. These altered properties may be advantageous for processes in the generation of fuels based on starch feed stocks.

Various modifications and variations of the described methods and system of the disclosure will be apparent to those skilled in the art without departing from the scope and spirit of the disclosure. Although the disclosure has been described in connection with specific representative embodiments, it should be understood that the subject matters as claimed should not be unduly limited to such specific embodiments. Indeed, various modifications of the described modes for carrying out the disclosure that are obvious to those skilled in the art are intended to be within the scope of the following claims.

1. A glucoamylase variant comprising amino acid substitutions corresponding to positions:
(a) 61, 417, 431, and 539;
(b) 43, 417, 431, 535, and 539; or
(c) 73, 503, and 563
of SEQ ID NO: 2, or equivalent positions in a parent glucoamylase, wherein the glucoamylase variant has at least 80% sequence identity with SEQ ID NO: 1 or 2, or the parent glucoamylase.

2. The glucoamylase variant of claim 1, wherein the amino acid substitutions are:
(a) N61I, L417G/R/V, A431L/Q, and A539R;
(b) I43Q/R, L417G/R/V, A431L/Q, A535R, and A539R; or
(c) G73F, E503V, and N563K.

3. The glucoamylase variant of claim 2, comprising one of the following sets of substitutions, at the relevant positions of SEQ ID NO: 2 or equivalent positions in a parent glucoamylase:
N61I/L417V/A431L/A539R;
I43Q/N61I/L417V/A431L/A539R;
N61I/L417V/A431L/A535R/A539R
I43 Q/L417V/A431L/A535R/A539R;
I43 Q/N61I/L417V/A431L/A535R/A539R;
I43 Q/N61I/L417V/T430A/A431L/A535R/A539R;
I43Q/L417V/T430A/A431L/Q511H/A535R/A539R/N5631;
N61I/L417V/T430A/A431L/Q511H/A535R/A539R/N5631;
I43Q/N61I/L417V/T430A/A431L/Q511H/A535R/A539R/N563 I;
I43R/N61I/L417V/A431L/A539R;
I43R/N61I/L417V/T430A/A43 μL/A535R/A539R;
G73F/L417R/E503 V/A539R/N563 K;
I43R/G73F/L417R/E503V/A539R/N563K; and
I43R/G73F/E503V/Q511H/N563K.

4. The glucoamylase variant of claim 1, wherein the parent glucoamylase or the glucoamylase variant has a catalytic domain that has at least 80% sequence identity with SEQ ID NO: 1, 2, 3, 5, 6, 7, 8, or 9.

5. The glucoamylase variant of claim 1, wherein the parent glucoamylase or the glucoamylase variant has a starch binding domain that has at least 95% sequence identity with SEQ ID NO: 1, 2, 11, 385, 386, 387, 388, 389, or 390.

6. The glucoamylase variant of claim 1, wherein the glucoamylase variant has at least 85% sequence identity with SEQ ID NO: 1 or 2.

7. The glucoamylase variant of claim 6, wherein the glucoamylase variant has at least 90% sequence identity with SEQ ID NO: 1 or 2.

8. The glucoamylase variant of claim 7, wherein the glucoamylase variant has at least 95% sequence identity with SEQ ID NO: 1 or 2.

9. The glucoamylase variant of claim 8, wherein the glucoamylase variant has at least 99.5% sequence identity with SEQ ID NO: 1 or 2.

10. The glucoamylase variant of claim 1, wherein the parent glucoamylase comprises SEQ ID NO: 1 or 2.

11. The glucoamylase variant of claim 10, wherein the parent glucoamylase consists of SEQ ID NO: 1 or 2.

12. The glucoamylase variant of claim 1, wherein the glucoamylase variant comprises one or more additional amino acid substitutions corresponding to position: 43, 44, 61, 73, 294, 430, 503, 511, 535, or 563 of SEQ ID NO: 2, or an equivalent position in the parent glucoamylase.

13. The glucoamylase variant of claim 12, wherein the amino acid substitutions are: I43Q/R, D44C/R, N61I, G73F, G294C, T430A/M, E503A/V, Q511H, A535R, or N563I/K of SEQ ID NO: 2, or an equivalent position in the parent glucoamylase.

14. The glucoamylase variant of claim 12, wherein the glucoamylase variant comprises one or more additional amino acid substitutions corresponding to position: 10, 14, 15, 23, 42, 59, 60, 65, 67, 68, 72, 97, 98, 99, 102, 110, 113, 114, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 418, 433, 436, 442, 444, 448, 451, 493, 494, 495, 502, 508, 518, 519, 520, 527, 531, 536, 537, or 577 of SEQ ID NO: 2, or an equivalent position in the parent glucoamylase.

15. The glucoamylase variant of claim 14, wherein the amino acid substitutions are: T10S, T42V, T67M, E68C/M, A72Y, S97N, S102A/M/R, K114M/Q, I133TN, N145I, N153A/D/E/M/S/V, T205Q, Q219S, W228A/F/H/MN, V229I/L, S230C/F/G/L/N/Q/R, 5231LN, D236R, I239V/Y, N263P, L264D/K, A268C/D/G/K, S291A/F/H/M/T, A301P/R, V338I/N/Q, T342V, S344M/P/Q/R/V, G361D/E/F/I/L/M/P/S/W/Y, A364D/E/F/G/K/L/M/R/S/TN/W, T375N, K394S, R433C/E/G/L/N/SN/Y, I436H, T451K, T495K/M/S, Q508R, A519I/K/R/Y, A520C/L/P, V531 L, V536M, or N577K/P/R of SEQ ID NO: 2, or an equivalent position in the parent glucoamylase.

16. The glucoamylase variant of claim 1, wherein the glucoamylase variant exhibits increased thermostability as compared to the parent glucoamylase.

17. The glucoamylase variant of claim 16, wherein the glucoamylase variant comprises one or more additional amino acid substitutions corresponding to positions: 10, 42, 59, 61, 68, 72, 73, 97, 98, 99, 102, 114, 133, 140, 144, 152, 153, 182, 204, 205, 214, 216, 228, 229, 230, 231, 236, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 430, 433, 436, 442, 444, 448, 451, 493, 495, 503, 508, 511, 518, 519, 520, 527, 531, 535, 536, 537, 563, or 577 of SEQ ID NO: 2, or an equivalent position in the parent glucoamylase.

18. The glucoamylase variant of claim 17, wherein the amino acid substitutions are: T10S, T42V, E68C/M, G73F/W, K114M/Q, I133V, N153A/E/M/SN, W228V, V229I/L, S230Q, S231V, D236R, L264D/K, A268D, S291A/E/H/M/T, A301P/R, V338I/N/Q, S344M/P/Q/R/V, G361D/E/F/I/L/M/P/S/W/Y, A364D/E/F/G/K/L/M/R/S/TN/W, T375N, R433c/E/G/L, R433N/SN, I436H, T495K/S, E503A/CN, Q508R, Q511H, A519K/R/Y, V531L, A535K/N/P/R, N563C/E/I/K/L/Q/TV, or N577K/P/R of SEQ ID NO: 2, or an equivalent position in the parent glucoamylase.

19. The glucoamylase variant of claim 1, wherein the glucoamylase variant exhibits increased specific activity as compared to the parent glucoamylase.

20. The glucoamylase variant of claim 19, wherein the glucoamylase variant comprises one or more additional amino acid substitutions corresponding to positions:
10, 14, 15, 23, 59, 60, 61, 65, 67, 68, 72, 73, 97, 98, 99, 102, 110, 113, 133, 140, 144, 145, 147, 152, 153, 164, 182, 204, 205, 214, 216, 219, 228, 229, 230, 231, 236, 239, 241, 242, 263, 264, 265, 268, 269, 276, 284, 291, 300, 301, 303, 311, 338, 342, 344, 346, 349, 359, 361, 364, 375, 379, 382, 390, 391, 393, 394, 410, 418, 430, 433, 442, 444, 448, 451, 493, 494, 495, 502, 503, 508, 511, 518, 519, 520, 531, 535, or 536 of SEQ ID NO: 2, or an equivalent position in the parent glucoamylase.

21. The glucoamylase variant of claim 19, wherein the amino acid substitutions corresponding to positions: N61I, T67M, A72Y, S97N, S102A/M/R, I133T, N145I, N153D, T205Q, Q219S, W228A/F/H/M, S230C/F/G/L/N/Q/R, S231L, I239V/Y, N263P, A268C/G/K, S291A, T342V, K394S, T430K, R433Y, T451K, T495M, A519I, A520C/L/P, A535R, or V536M of SEQ ID NO: 2, or an equivalent position in the parent glucoamylase.

22. The glucoamylase variant of claim 1, wherein the glucoamylase variant exhibits both increased thermostability and increased specific activity as compared to the parent glucoamylase.

23. The glucoamylase variant of claim 22, wherein the glucoamylase variant comprises additional one or more amino acid substitutions corresponding to positions: 10, 15, 59, 61, 68, 72, 73, 97, 99, 102, 140, 153, 182, 204, 205, 214, 228, 229, 230, 231, 236, 241, 242, 264, 265, 268, 276, 284, 291, 300, 301, 303, 311, 338, 344, 346, 349, 359, 361, 364, 375, 379, 382, 391, 393, 394, 410, 430, 433, 444, 448, 451, 495, 503, 511, 520, 531, 535, or 536 of SEQ ID NO: 2, or an equivalent position in the parent glucoamylase.

24. The glucoamylase variant of claim 22, wherein the amino acid substitutions are: W228F/H/M, S230C/F/G/N/Q/R, S231L, A268C/D/G/K, S291A, R433Y, S451K, E503C, Q511H, A520C/L/P, or A535N/P/R of SEQ ID NO: 2, or an equivalent position in the parent glucoamylase.

25. A glucoamylase variant comprising one of the following sets of substitutions, at positions of SEQ ID NO: 2 or equivalent positions in a parent glucoamylase:
L417V/A431L/A539R;
I43Q/L417V/A431L/A539R;
L417V/A431L/A535R/A539R
I43R/L417V/A431L/A539R;
L417R/A431L/A539R; or
L417G/A431L/A539R;
wherein the glucoamylase variant does not have any further substitutions relative to the parent glucoamylase; and
wherein the parent glucoamylase has a catalytic domain that has at least 80% sequence identity with SEQ ID NO: 1, 2, 3, 5, 6, 7, 8, or 9.

26. The glucoamylase variant of claim 25 wherein the parent glucoamylase has a starch binding domain that has at least 95% sequence identity with SEQ ID NO: 1, 2, 11, 385, 386, 387, 388, 389, or 390.

27. The glucoamylase variant of claim 25 wherein the parent glucoamylase has at least 80% sequence identity with SEQ ID NO: 1 or 2.

28. The glucoamylase variant of claim 25, wherein the parent glucoamylase comprises SEQ ID NO: 1 or 2.

29. The glucoamylase variant of claim 28, wherein the parent glucoamylase consists of SEQ ID NO: 1 or 2.

30. The glucoamylase variant of claim 25, wherein the glucoamylase variant exhibits increased thermostability and/or increased specific activity as compared to the parent glucoamylase.

31. The glucoamylase variant of claim 1, wherein the parent glucoamylase is selected from a glucoamylase obtained from a Trichoderma spp., an Aspergillus spp., a Humicola spp., a Penicillium spp., a Talaromycese spp., or a Schizosaccharmyces spp.

32. The glucoamylase variant of claim 31, wherein the parent glucoamylase is obtained from a Trichoderma spp. or an Aspergillus spp.

33. A polynucleotide encoding the variant of claim 1.

34. A vector comprising the polynucleotide of claim 33.

35. A host cell comprising the vector of claim 34.

36. A method of producing a glucoamylase variant in a host cell comprising culturing the host cell of claim 35 under conditions suitable for the expression and production of the glucoamylase variant and producing the glucoamylase variant.

37. The method according to claim 36 further comprising recovering the glucoamylase variant from the culture.

38. An enzyme composition comprising the glucoamylase variant of claim 1.

39. A method of converting a starch comprising using the enzyme composition of claim 38 to hydrolyze a starch.

40. A method of fermenting an alcohol comprising using the enzyme composition of claim 38 to ferment an alcohol.

41. A method of producing a high glucose syrup comprising using the enzyme composition of claim 38 to produce a high glucose syrup.


SEQUENCE LISTING (1097 sequences)

SEQ ID NO: 1 - protein, 632 aa, Trichoderma reesei
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 2 - protein, 599 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 3 - protein, 453 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTC

SEQ ID NO: 4 - DNA, 1899 bp, Trichoderma reesei
atgcacgtcctgtcgactgcggtgctgctcggctccgttgccgttcaaaaggtcctgggaagaccaggatcaagcggtctgtccgacgtcaccaagaggtctgttgacgacttcatcagcaccgagacgcctattgcactgaacaatcttctttgcaatgttggtcctgatggatgccgtgcattcggcacatcagctggtgcggtgattgcatctcccagcacaattgacccggactactattacatgtggacgcgagatagcgctcttgtcttcaagaacctcatcgaccgcttcaccgaaacgtacgatgcgggcctgcagcgccgcatcgagcagtacattactgcccaggtcactctccagggcctctctaacccctcgggctccctcgcggacggctctggtctcggcgagcccaagtttgagttgaccctgaagcctttcaccggcaactggggtcgaccgcagcgggatggcccagctctgcgagccattgccttgattggatactcaaagtggctcatcaacaacaactatcagtcgactgtgtccaacgtcatctggcctattgtgcgcaacgacctcaactatgttgcccagtactggaaccaaaccggctttgacctctgggaagaagtcaatgggagctcattctttactgttgccaaccagcaccgagcacttgtcgagggcgccactcttgctgccactcttggccagtcgggaagcgcttattcatctgttgctccccaggttttgtgctttctccaacgattctgggtgtcgtctggtggatacgtcgactccaacatcaacaccaacgagggcaggactggcaaggatgtcaactccgtcctgacttccatccacaccttcgatcccaaccttggctgtgacgcaggcaccttccagccatgcagtgacaaagcgctctccaacctcaaggttgttgtcgactccttccgctccatctacggcgtgaacaagggcattcctgccggtgctgccgtcgccattggccggtatgcagaggatgtgtactacaacggcaacccttggtatcttgctacatttgctgctgccgagcagctgtacgatgccatctacgtctggaagaagacgggctccatcacggtgaccgccacctccctggccttcttccaggagcttgttcctggcgtgacggccgggacctactccagcagctcttcgacctttaccaacatcatcaacgccgtctcgacatacgccgatggcttcctcagcgaggctgccaagtacgtccccgccgacggttcgctggccgagcagtttgaccgcaacagcggcactccgctgtctgcgcttcacctgacgtggtcgtacgcctcgttcttgacagccacggcccgtcgggctggcatcgtgcccccctcgtgggccaacagcagcgctagcacgatcccctcgacgtgctccggcgcgtccgtggtcggatcctactcgcgtcccaccgccacgtcattccctccgtcgcagacgcccaagcctggcgtgccttccggtactccctacacgcccctgccctgcgcgaccccaacctccgtggccgtcaccttccacgagctcgtgtcgacacagtttggccagacggtcaaggtggcgggcaacgccgcggccctgggcaactggagcacgagcgccgccgtggctctggacgccgtcaactatgccgataaccaccccctgtggattgggacggtcaacctcgaggctggagacgtcgtggagtacaagtacatcaatgtgggccaagatggctccgtgacctgggagagtgatcccaaccacacttacacggttcctgcggtggcttgtgtgacgcaggttgtcaaggaggacacctggcagtcgtaa

SEQ ID NO: 5 - protein, 448 aa, Aspergillus awamori
ATLDSWLSNEATVARTAILNNIGADGAWVSGADSGIVVASPSTDNPDYFYTWTRDSGLVIKTLVDLFRNGDTDLLSTIENYISSQAIVQGISNPSGDLSSGGLGEPKFNVDETAYTGSWGRPQRDGPALRATAMIGFRQWLLDNGYTSAATEIVWPLVRNDLSYVAQYWNQTGYDLWEEVNGSSFFTIAVQHRALVEGSAFATAVGSSCSWCDSQAPQILCYLQSFWTGEYILANFDSSRSGKDTNTLLGSIHTFDPEAGCDDSTFQPCSPRALANHKEVVDSFRSIYTLNDGLSDSEAVAVGRYPKDSYYNGNPWFLCTLAAAEQLYDALYQWDKQGSLEITDVSLDFFQALYSDAATGTYSSSSSTYSSIVDAVKTFADGFVSIVETHAASNGSLSEQYDKSDGDELSARDLTWSYAALLTANNRRNSVMPPSWGETSASSVPGTC

SEQ ID NO: 6 - protein, 449 aa, Aspergillus niger
ATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTC

SEQ ID NO: 7 - protein, 450 aa, Aspergillus oryzae
QSDLNAFIEAQTPIAKQGYLNNIGADGKLVEGAAAGIVYASPSKSNPDYFYTWTRDAGLTMEEYIEQFIGGDATLESTIQNYVDSQANEQAVSNPSGGLSDGSGLAEPKFYYNISQFTDSWGRPQRDGPALRASALIAYGNSLISSDKQSVVKANIWPIYQNDLSYVGQYWNQTGFDLWEEVQGSSFFTVAVQHKALVEGDAFAKALGEECQACSVAPQILCHLQDFWNGSAVLSNLPTNGRSGLDTNSLLGSIHTFDPAAACDDTTFQPCSSRALSNHKLVVDSFRSVYGINNGRGAGKAAAVGPYAEDTYQGGNPWYLTTLVAAELLYDALYQWDKQGQVNVTETSLPFFKDLSSNVTTGSYAKSSSAYESLTSAVKTYADGFISVVQEYTPDGGALAEQYSRDQGTPVSASDLTWSYAAFLSAVGRRNGTVPASWGSSTANAVPSQC

SEQ ID NO: 8 - protein, 441 aa, Humicola grisea
AAVDTFINTEKPIAWNKLLANIGPNGKAAPGAAAGVVIASPSRTDPPYFFTWTPDAALVLTGIIESLGHNYNTTLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECPPCTTVAPQVLCFQQAFWNSKGNYVVSTSTAGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTC

SEQ ID NO: 9 - protein, 452 aa, Hypocrea vinosa
SVDDFINTQTPIALNNLLCNVGPDGCRAFGTSAGAVIASPSTTDPDYYYMWTRDSALVFKNIVDRFTQQYDAGLQRRIEQYISAQVTLQGISNPSGSLSDGSGLGEPKFELTLSQFTGNWGRPQRDGPALRAIALIGYSKWLINNNYQSTVSNIIWPIVRNDLNYVAQYWNQTGFDLWEEVNGSSFFTVANQHRALVEGATLAATLGQSGSTYSSVAPQILCFLQRFWVSGGYIDSNINTNEGRTGKDANSLLASIHTFDPSLGCDASTFQPCSDKALSNLKVVVDSFRSIYGVNKGIPAGSAVAIGRYPEDVYFNGNPWYLATFAAAEQLYDSVYVWKKTGSITVTSTSSAFFQELVPGVAAGTYSSSQSTFTSIINAISTYADGFLSEAAKYVPADGSLAEQFDRNTGTPLSAVHLTWSYASFLTAAARRAGVVPPSWASSGANTVPSSC

SEQ ID NO: 10 - protein, 37 aa, Trichoderma reesei
SGASVVGSYSRPTATSFPPSQTPKPGVPSGTPYTPLP

SEQ ID NO: 11 - protein, 109 aa, Trichoderma reesei
CATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 12 - protein, 6 aa, Trichoderma reesei
SVDDFI

SEQ ID NO: 13 - DNA, 25 bp, Artificial
  Synthetic primer
tcgcgttaacgctagcatggatctc

SEQ ID NO: 14 - DNA, 25 bp, Artificial
  Synthetic primer
tcgcgttaacgctagcatggatctc

SEQ ID NO: 15 - DNA, 45 bp, Artificial
  Synthetic primer
cgtcaccaagaggtctgttgacnnsttcatcagcaccgagacgcc

SEQ ID NO: 16 - DNA, 25 bp, Artificial
  Synthetic primer
gtcaacagacctcttggtgacgtcg

SEQ ID NO: 17 - DNA, 48 bp, Artificial
  Synthetic primer
caccaagaggtctgttgacgacnnsatcagcaccgagacgcctattgc

SEQ ID NO: 18 - DNA, 24 bp, Artificial
  Synthetic primer
gtcgtcaacagacctcttggtgac

SEQ ID NO: 19 - DNA, 37 bp, Artificial
  Synthetic primer
tgacgacttcatcagcaccgagnnscctattgcactg

SEQ ID NO: 20 - DNA, 21 bp, Artificial
  Synthetic primer
ctcggtgctgatgaagtcgtc

SEQ ID NO: 21 - DNA, 45 bp, Artificial
  Synthetic primer
tcatcagcaccgagacgcctnnsgcactgaacaatcttctttgca

SEQ ID NO: 22 - DNA, 25 bp, Artificial
  Synthetic primer
aggcgtctcggtgctgatgaagtcg

SEQ ID NO: 23 - DNA, 37 bp, Artificial
  Synthetic primer
cagcaccgagacgcctattgcannsaacaatcttctt

SEQ ID NO: 24 - DNA, 21 bp, Artificial
  Synthetic primer
tgcaataggcgtctcggtgct

SEQ ID NO: 25 - DNA, 37 bp, Artificial
  Synthetic primer
caccgagacgcctattgcactgnnsaatcttctttgc

SEQ ID NO: 26 - DNA, 21 bp, Artificial
  Synthetic primer
cagtgcaataggcgtctcggt

SEQ ID NO: 27 - DNA, 37 bp, Artificial
  Synthetic primer
caatcttctttgcaatgttggtnnsgatggatgccgt

SEQ ID NO: 28 - DNA, 22 bp, Artificial
  Synthetic primer
accaacattgcaaagaagattg

SEQ ID NO: 29 - DNA, 45 bp, Artificial
  Synthetic primer
ttctttgcaatgttggtcctnnsggatgccgtgcattcggcacat

SEQ ID NO: 30 - DNA, 25 bp, Artificial
  Synthetic primer
aggaccaacattgcaaagaagattg

SEQ ID NO: 31 - DNA, 45 bp, Artificial
  Synthetic primer
gtcctgatggatgccgtgcannsggcacatcagctggtgcggtga

SEQ ID NO: 32 - DNA, 25 bp, Artificial
  Synthetic primer
tgcacggcatccatcaggaccaaca

SEQ ID NO: 33 - DNA, 37 bp, Artificial
  Synthetic primer
tgcggtgattgcatctcccagcnnsattgacccggac

SEQ ID NO: 34 - DNA, 22 bp, Artificial
  Synthetic primer
gctgggagatgcaatcaccgca

SEQ ID NO: 35 - DNA, 45 bp, Artificial
  Synthetic primer
tgattgcatctcccagcacannsgacccggactactattacatgt

SEQ ID NO: 36 - DNA, 25 bp, Artificial
  Synthetic primer
tgtgctgggagatgcaatcaccgca

SEQ ID NO: 37 - DNA, 45 bp, Artificial
  Synthetic primer
ttgcatctcccagcacaattnnsccggactactattacatgtgga

SEQ ID NO: 38 - DNA, 25 bp, Artificial
  Synthetic primer
aattgtgctgggagatgcaatcacc

SEQ ID NO: 39 - DNA, 45 bp, Artificial
  Synthetic primer
catctcccagcacaattgacnnsgactactattacatgtggacgc

SEQ ID NO: 40 - DNA, 25 bp, Artificial
  Synthetic primer
gtcaattgtgctgggagatgcaatc

SEQ ID NO: 41 - DNA, 46 bp, Artificial
  Synthetic primer
ctcccagcacaattgacccgnnstactattacatgtggacgcgaga

SEQ ID NO: 42 - DNA, 25 bp, Artificial
  Synthetic primer
cgggtcaattgtgctgggagatgca

SEQ ID NO: 43 - DNA, 45 bp, Artificial
  Synthetic primer
ccagcacaattgacccggacnnstattacatgtggacgcgagata

SEQ ID NO: 44 - DNA, 25 bp, Artificial
  Synthetic primer
gtccgggtcaattgtgctgggagat

SEQ ID NO: 45 - DNA, 45 bp, Artificial
  Synthetic primer
caattgacccggactactatnnsatgtggacgcgagatagcgctc

SEQ ID NO: 46 - DNA, 25 bp, Artificial
  Synthetic primer
atagtagtccgggtcaattgtgctg

SEQ ID NO: 47 - DNA, 45 bp, Artificial
  Synthetic primer
acccggactactattacatgnnsacgcgagatagcgctcttgtct

SEQ ID NO: 48 - DNA, 25 bp, Artificial
  Synthetic primer
catgtaatagtagtccgggtcaatt

SEQ ID NO: 49 - DNA, 37 bp, Artificial
  Synthetic primer
gacgcgagatagcgctcttgtcnnsaagaacctcatc

SEQ ID NO: 50 - DNA, 21 bp, Artificial
  Synthetic primer
gacaagagcgctatctcgcgt

SEQ ID NO: 51 - DNA, 37 bp, Artificial
  Synthetic primer
gcgagatagcgctcttgtcttcnnsaacctcatcgac

SEQ ID NO: 52 - DNA, 21 bp, Artificial
  Synthetic primer
gaagacaagagcgctatctcg

SEQ ID NO: 53 - DNA, 37 bp, Artificial
  Synthetic primer
agatagcgctcttgtcttcaagnnsctcatcgaccgc

SEQ ID NO: 54 - DNA, 21 bp, Artificial
  Synthetic primer
cttgaagacaagagcgctatc

SEQ ID NO: 55 - DNA, 37 bp, Artificial
  Synthetic primer
tgtcttcaagaacctcatcgacnnsttcaccgaaacg

SEQ ID NO: 56 - DNA, 21 bp, Artificial
  Synthetic primer
gtcgatgaggttcttgaagac

SEQ ID NO: 57 - DNA, 37 bp, Artificial
  Synthetic primer
caagaacctcatcgaccgcttcnnsgaaacgtacgat

SEQ ID NO: 58 - DNA, 21 bp, Artificial
  Synthetic primer
gaagcggtcgatgaggttctt

SEQ ID NO: 59 - DNA, 37 bp, Artificial
  Synthetic primer
gaacctcatcgaccgcttcaccnnsacgtacgatgcg

SEQ ID NO: 60 - DNA, 21 bp, Artificial
  Synthetic primer
ggtgaagcggtcgatgaggtt

SEQ ID NO: 61 - DNA, 45 bp, Artificial
  Synthetic primer
tcgaccgcttcaccgaaacgnnsgatgcgggcctgcagcgccgca

SEQ ID NO: 62 - DNA, 25 bp, Artificial
  Synthetic primer
cgtttcggtgaagcggtcgatgagg

SEQ ID NO: 63 - DNA, 37 bp, Artificial
  Synthetic primer
ccgcttcaccgaaacgtacgatnnsggcctgcagcgc

SEQ ID NO: 64 - DNA, 22 bp, Artificial
  Synthetic primer
atcgtacgtttcggtgaagcgg

SEQ ID NO: 65 - DNA, 37 bp, Artificial
  Synthetic primer
cttcaccgaaacgtacgatgcgnnsctgcagcgccgc

SEQ ID NO: 66 - DNA, 21 bp, Artificial
  Synthetic primer
cgcatcgtacgtttcggtgaa

SEQ ID NO: 67 - DNA, 45 bp, Artificial
  Synthetic primer
aaacgtacgatgcgggcctgnnscgccgcatcgagcagtacatta

SEQ ID NO: 68 - DNA, 25 bp, Artificial
  Synthetic primer
caggcccgcatcgtacgtttcggtg

SEQ ID NO: 69 - DNA, 45 bp, Artificial
  Synthetic primer
cgtacgatgcgggcctgcagnnscgcatcgagcagtacattactg

SEQ ID NO: 70 - DNA, 25 bp, Artificial
  Synthetic primer
ctgcaggcccgcatcgtacgtttcg

SEQ ID NO: 71 - DNA, 45 bp, Artificial
  Synthetic primer
ctctccagggcctctctaacnnstcgggctccctcgcggacggct

SEQ ID NO: 72 - DNA, 25 bp, Artificial
  Synthetic primer
gttagagaggccctggagagtgacc

SEQ ID NO: 73 - DNA, 37 bp, Artificial
  Synthetic primer
gggcctctctaacccctcgggcnnsctcgcggacggc

SEQ ID NO: 74 - DNA, 21 bp, Artificial
  Synthetic primer
gcccgaggggttagagaggcc

SEQ ID NO: 75 - DNA, 37 bp, Artificial
  Synthetic primer
cctctctaacccctcgggctccnnsgcggacggctct

SEQ ID NO: 76 - DNA, 21 bp, Artificial
  Synthetic primer
ggagcccgaggggttagagag

SEQ ID NO: 77 - DNA, 37 bp, Artificial
  Synthetic primer
ctctaacccctcgggctccctcnnsgacggctctggt

SEQ ID NO: 78 - DNA, 21 bp, Artificial
  Synthetic primer
gagggagcccgaggggttaga

SEQ ID NO: 79 - DNA, 45 bp, Artificial
  Synthetic primer
acccctcgggctccctcgcgnnsggctctggtctcggcgagccca

SEQ ID NO: 80 - DNA, 25 bp, Artificial
  Synthetic primer
cgcgagggagcccgaggggttagag

SEQ ID NO: 81 - DNA, 37 bp, Artificial
  Synthetic primer
ctcgggctccctcgcggacggcnnsggtctcggcgag

SEQ ID NO: 82 - DNA, 21 bp, Artificial
  Synthetic primer
gccgtccgcgagggagcccga

SEQ ID NO: 83 - DNA, 37 bp, Artificial
  Synthetic primer
tggtctcggcgagcccaagtttnnsttgaccctgaag

SEQ ID NO: 84 - DNA, 22 bp, Artificial
  Synthetic primer
aaacttgggctcgccgagacca

SEQ ID NO: 85 - DNA, 37 bp, Artificial
  Synthetic primer
tctcggcgagcccaagtttgagnnsaccctgaagcct

SEQ ID NO: 86 - DNA, 21 bp, Artificial
  Synthetic primer
ctcaaacttgggctcgccgag

SEQ ID NO: 87 - DNA, 37 bp, Artificial
  Synthetic primer
cgagcccaagtttgagttgaccnnsaagcctttcacc

SEQ ID NO: 88 - DNA, 21 bp, Artificial
  Synthetic primer
ggtcaactcaaacttgggctc

SEQ ID NO: 89 - DNA, 45 bp, Artificial
  Synthetic primer
ccaagtttgagttgaccctgnnscctttcaccggcaactggggtc

SEQ ID NO: 90 - DNA, 25 bp, Artificial
  Synthetic primer
cagggtcaactcaaacttgggctcg

SEQ ID NO: 91 - DNA, 46 bp, Artificial
  Synthetic primer
ttgagttgaccctgaagcctnnsaccggcaactggggtcgaccgca

SEQ ID NO: 92 - DNA, 25 bp, Artificial
  Synthetic primer
aggcttcagggtcaactcaaacttg

SEQ ID NO: 93 - DNA, 45 bp, Artificial
  Synthetic primer
ccctgaagcctttcaccggcnnstggggtcgaccgcagcgggatg

SEQ ID NO: 94 - DNA, 25 bp, Artificial
  Synthetic primer
gccggtgaaaggcttcagggtcaac

SEQ ID NO: 95 - DNA, 45 bp, Artificial
  Synthetic primer
ctttcaccggcaactggggtnnsccgcagcgggatggcccagctc

SEQ ID NO: 96 - DNA, 25 bp, Artificial
  Synthetic primer
accccagttgccggtgaaaggcttc

SEQ ID NO: 97 - DNA, 45 bp, Artificial
  Synthetic primer
gcaactggggtcgaccgcagnnsgatggcccagctctgcgagcca

SEQ ID NO: 98 - DNA, 25 bp, Artificial
  Synthetic primer
ctgcggtcgaccccagttgccggtg

SEQ ID NO: 99 - DNA, 37 bp, Artificial
  Synthetic primer
ggatggcccagctctgcgagccnnsgccttgattgga

SEQ ID NO: 100 - DNA, 22 bp, Artificial
  Synthetic primer
ggctcgcagagctgggccatcc

SEQ ID NO: 101 - DNA, 45 bp, Artificial
  Synthetic primer
tgcgagccattgccttgattnnstactcaaagtggctcatcaaca

SEQ ID NO: 102 - DNA, 25 bp, Artificial
  Synthetic primer
aatcaaggcaatggctcgcagagct

SEQ ID NO: 103 - DNA, 37 bp, Artificial
  Synthetic primer
cattgccttgattggatactcannstggctcatcaac

SEQ ID NO: 104 - DNA, 22 bp, Artificial
  Synthetic primer
tgagtatccaatcaaggcaatg

SEQ ID NO: 105 - DNA, 37 bp, Artificial
  Synthetic primer
tggatactcaaagtggctcatcnnsaacaactatcag

SEQ ID NO: 106 - DNA, 21 bp, Artificial
  Synthetic primer
gatgagccactttgagtatcc

SEQ ID NO: 107 - DNA, 37 bp, Artificial
  Synthetic primer
atactcaaagtggctcatcaacnnsaactatcagtcg

SEQ ID NO: 108 - DNA, 21 bp, Artificial
  Synthetic primer
gttgatgagccactttgagta

SEQ ID NO: 109 - DNA, 45 bp, Artificial
  Synthetic primer
caaagtggctcatcaacaacnnstatcagtcgactgtgtccaacg

SEQ ID NO: 110 - DNA, 25 bp, Artificial
  Synthetic primer
gttgttgatgagccactttgagtat

SEQ ID NO: 111 - DNA, 37 bp, Artificial
  Synthetic primer
aaagtggctcatcaacaacaacnnscagtcgactgtg

SEQ ID NO: 112 - DNA, 21 bp, Artificial
  Synthetic primer
gttgttgttgatgagccactt

SEQ ID NO: 113 - DNA, 45 bp, Artificial
  Synthetic primer
ggctcatcaacaacaactatnnstcgactgtgtccaacgtcatct

SEQ ID NO: 114 - DNA, 25 bp, Artificial
  Synthetic primer
atagttgttgttgatgagccacttt

SEQ ID NO: 115 - DNA, 37 bp, Artificial
  Synthetic primer
caacaactatcagtcgactgtgnnsaacgtcatctgg

SEQ ID NO: 116 - DNA, 21 bp, Artificial
  Synthetic primer
cacagtcgactgatagttgtt

SEQ ID NO: 117 - DNA, 37 bp, Artificial
  Synthetic primer
caactatcagtcgactgtgtccnnsgtcatctggcct

SEQ ID NO: 118 - DNA, 21 bp, Artificial
  Synthetic primer
ggacacagtcgactgatagtt

SEQ ID NO: 119 - DNA, 38 bp, Artificial
  Synthetic primer
gcctattgtgcgcaacgacctcnnstatgttgcccagt

SEQ ID NO: 120 - DNA, 21 bp, Artificial
  Synthetic primer
gaggtcgttgcgcacaatagg

SEQ ID NO: 121 - DNA, 45 bp, Artificial
  Synthetic primer
acctcaactatgttgcccagnnstggaaccaaaccggctttgacc

SEQ ID NO: 122 - DNA, 25 bp, Artificial
  Synthetic primer
ctgggcaacatagttgaggtcgttg

SEQ ID NO: 123 - DNA, 45 bp, Artificial
  Synthetic primer
atgttgcccagtactggaacnnsaccggctttgacctctgggaag

SEQ ID NO: 124 - DNA, 25 bp, Artificial
  Synthetic primer
gttccagtactgggcaacatagttg

SEQ ID NO: 125 - DNA, 45 bp, Artificial
  Synthetic primer
agtactggaaccaaaccggcnnsgacctctgggaagaagtcaatg

SEQ ID NO: 126 - DNA, 25 bp, Artificial
  Synthetic primer
gccggtttggttccagtactgggca

SEQ ID NO: 127 - DNA, 45 bp, Artificial
  Synthetic primer
accaaaccggctttgacctcnnsgaagaagtcaatgggagctcat

SEQ ID NO: 128 - DNA, 25 bp, Artificial
  Synthetic primer
gaggtcaaagccggtttggttccag

SEQ ID NO: 129 - DNA, 45 bp, Artificial
  Synthetic primer
ccggctttgacctctgggaannsgtcaatgggagctcattcttta

SEQ ID NO: 130 - DNA, 25 bp, Artificial
  Synthetic primer
ttcccagaggtcaaagccggtttgg

SEQ ID NO: 131 - DNA, 45 bp, Artificial
  Synthetic primer
gctttgacctctgggaagaannsaatgggagctcattctttactg

SEQ ID NO: 132 - DNA, 25 bp, Artificial
  Synthetic primer
ttcttcccagaggtcaaagccggtt

SEQ ID NO: 133 - DNA, 37 bp, Artificial
  Synthetic primer
ctttgacctctgggaagaagtcnnsgggagctcattc

SEQ ID NO: 134 - DNA, 22 bp, Artificial
  Synthetic primer
gacttcttcccagaggtcaaag

SEQ ID NO: 135 - DNA, 37 bp, Artificial
  Synthetic primer
tgtcgagggcgccactcttgctnnsactcttggccag

SEQ ID NO: 136 - DNA, 21 bp, Artificial
  Synthetic primer
agcaagagtggcgccctcgac

SEQ ID NO: 137 - DNA, 37 bp, Artificial
  Synthetic primer
cgagggcgccactcttgctgccnnscttggccagtcg

SEQ ID NO: 138 - DNA, 21 bp, Artificial
  Synthetic primer
ggcagcaagagtggcgccctc

SEQ ID NO: 139 - DNA, 45 bp, Artificial
  Synthetic primer
ctcttgctgccactcttggcnnstcgggaagcgcttattcatctg

SEQ ID NO: 140 - DNA, 25 bp, Artificial
  Synthetic primer
gccaagagtggcagcaagagtggcg

SEQ ID NO: 141 - DNA, 45 bp, Artificial
  Synthetic primer
ccactcttggccagtcgggannsgcttattcatctgttgctcccc

SEQ ID NO: 142 - DNA, 25 bp, Artificial
  Synthetic primer
tcccgactggccaagagtggcagca

SEQ ID NO: 143 - DNA, 37 bp, Artificial
  Synthetic primer
tggccagtcgggaagcgcttatnnstctgttgctccc

SEQ ID NO: 144 - DNA, 21 bp, Artificial
  Synthetic primer
ataagcgcttcccgactggcc

SEQ ID NO: 145 - DNA, 37 bp, Artificial
  Synthetic primer
gtcgggaagcgcttattcatctnnsgctccccaggtt

SEQ ID NO: 146 - DNA, 21 bp, Artificial
  Synthetic primer
agatgaataagcgcttcccga

SEQ ID NO: 147 - DNA, 37 bp, Artificial
  Synthetic primer
cgcttattcatctgttgctcccnnsgttttgtgcttt

SEQ ID NO: 148 - DNA, 21 bp, Artificial
  Synthetic primer
gggagcaacagatgaataagc

SEQ ID NO: 149 - DNA, 45 bp, Artificial
  Synthetic primer
tgtgctttctccaacgattcnnsgtgtcgtctggtggatacgtcg

SEQ ID NO: 150 - DNA, 26 bp, Artificial
  Synthetic primer
gaatcgttggagaaagcacaaaacct

SEQ ID NO: 151 - DNA, 37 bp, Artificial
  Synthetic primer
gtgctttctccaacgattctggnnstcgtctggtgga

SEQ ID NO: 152 - DNA, 21 bp, Artificial
  Synthetic primer
ccagaatcgttggagaaagca

SEQ ID NO: 153 - DNA, 38 bp, Artificial
  Synthetic primer
ctttctccaacgattctgggtgnnstctggtggatacg

SEQ ID NO: 154 - DNA, 21 bp, Artificial
  Synthetic primer
cacccagaatcgttggagaaa

SEQ ID NO: 155 - DNA, 37 bp, Artificial
  Synthetic primer
tctccaacgattctgggtgtcgnnsggtggatacgtc

SEQ ID NO: 156 - DNA, 22 bp, Artificial
  Synthetic primer
cgacacccagaatcgttggaga

SEQ ID NO: 157 - DNA, 39 bp, Artificial
  Synthetic primer
ggtgtcgtctggtggatacgtcnnstccaacatcaacac

SEQ ID NO: 158 - DNA, 21 bp, Artificial
  Synthetic primer
gacgtatccaccagacgacac

SEQ ID NO: 159 - DNA, 37 bp, Artificial
  Synthetic primer
tggtggatacgtcgactccaacnnsaacaccaacgag

SEQ ID NO: 160 - DNA, 21 bp, Artificial
  Synthetic primer
gttggagtcgacgtatccacc

SEQ ID NO: 161 - DNA, 38 bp, Artificial
  Synthetic primer
tggatacgtcgactccaacatcnnsaccaacgagggca

SEQ ID NO: 162 - DNA, 22 bp, Artificial
  Synthetic primer
gatgttggagtcgacgtatcca

SEQ ID NO: 163 - DNA, 39 bp, Artificial
  Synthetic primer
atacgtcgactccaacatcaacnnsaacgagggcaggac

SEQ ID NO: 164 - DNA, 21 bp, Artificial
  Synthetic primer
gttgatgttggagtcgacgta

SEQ ID NO: 165 - DNA, 45 bp, Artificial
  Synthetic primer
tcgactccaacatcaacaccnnsgagggcaggactggcaaggatg

SEQ ID NO: 166 - DNA, 25 bp, Artificial
  Synthetic primer
ggtgttgatgttggagtcgacgtat

SEQ ID NO: 167 - DNA, 45 bp, Artificial
  Synthetic primer
actccaacatcaacaccaacnnsggcaggactggcaaggatgtca

SEQ ID NO: 168 - DNA, 25 bp, Artificial
  Synthetic primer
gttggtgttgatgttggagtcgacg

SEQ ID NO: 169 - DNA, 37 bp, Artificial
  Synthetic primer
ctccaacatcaacaccaacgagnnsaggactggcaag

SEQ ID NO: 170 - DNA, 23 bp, Artificial
  Synthetic primer
ctcgttggtgttgatgttggagt

SEQ ID NO: 171 - DNA, 45 bp, Artificial
  Synthetic primer
acatcaacaccaacgagggcnnsactggcaaggatgtcaactccg

SEQ ID NO: 172 - DNA, 26 bp, Artificial
  Synthetic primer
gccctcgttggtgttgatgttggagt

SEQ ID NO: 173 - DNA, 37 bp, Artificial
  Synthetic primer
ttccatccacaccttcgatcccnnscttggctgtgac

SEQ ID NO: 174 - DNA, 21 bp, Artificial
  Synthetic primer
gggatcgaaggtgtggatgga

SEQ ID NO: 175 - DNA, 37 bp, Artificial
  Synthetic primer
catccacaccttcgatcccaacnnsggctgtgacgca

SEQ ID NO: 176 - DNA, 21 bp, Artificial
  Synthetic primer
gttgggatcgaaggtgtggat

SEQ ID NO: 177 - DNA, 37 bp, Artificial
  Synthetic primer
ccacaccttcgatcccaaccttnnstgtgacgcaggc

SEQ ID NO: 178 - DNA, 21 bp, Artificial
  Synthetic primer
aaggttgggatcgaaggtgtg

SEQ ID NO: 179 - DNA, 38 bp, Artificial
  Synthetic primer
cgatcccaaccttggctgtgacnnsggcaccttccagc

SEQ ID NO: 180 - DNA, 21 bp, Artificial
  Synthetic primer
gtcacagccaaggttgggatc

SEQ ID NO: 181 - DNA, 37 bp, Artificial
  Synthetic primer
tcccaaccttggctgtgacgcannsaccttccagcca

SEQ ID NO: 182 - DNA, 21 bp, Artificial
  Synthetic primer
tgcgtcacagccaaggttggg

SEQ ID NO: 183 - DNA, 37 bp, Artificial
  Synthetic primer
aggcaccttccagccatgcagtnnsaaagcgctctcc

SEQ ID NO: 184 - DNA, 21 bp, Artificial
  Synthetic primer
actgcatggctggaaggtgcc

SEQ ID NO: 185 - DNA, 38 bp, Artificial
  Synthetic primer
caaagcgctctccaacctcaagnnsgttgtcgactcct

SEQ ID NO: 186 - DNA, 21 bp, Artificial
  Synthetic primer
cttgaggttggagagcgcttt

SEQ ID NO: 187 - DNA, 37 bp, Artificial
  Synthetic primer
ggttgttgtcgactccttccgcnnsatctacggcgtg

SEQ ID NO: 188 - DNA, 21 bp, Artificial
  Synthetic primer
gcggaaggagtcgacaacaac

SEQ ID NO: 189 - DNA, 45 bp, Artificial
  Synthetic primer
ttgtcgactccttccgctccnnstacggcgtgaacaagggcattc

SEQ ID NO: 190 - DNA, 25 bp, Artificial
  Synthetic primer
ggagcggaaggagtcgacaacaacc

SEQ ID NO: 191 - DNA, 45 bp, Artificial
  Synthetic primer
actccttccgctccatctacnnsgtgaacaagggcattcctgccg

SEQ ID NO: 192 - DNA, 25 bp, Artificial
  Synthetic primer
gtagatggagcggaaggagtcgaca

SEQ ID NO: 193 - DNA, 45 bp, Artificial
  Synthetic primer
gctccatctacggcgtgaacnnsggcattcctgccggtgctgccg

SEQ ID NO: 194 - DNA, 25 bp, Artificial
  Synthetic primer
gttcacgccgtagatggagcggaag

SEQ ID NO: 195 - DNA, 38 bp, Artificial
  Synthetic primer
ctacggcgtgaacaagggcattnnsgccggtgctgccg

SEQ ID NO: 196 - DNA, 21 bp, Artificial
  Synthetic primer
aatgcccttgttcacgccgta

SEQ ID NO: 197 - DNA, 37 bp, Artificial
  Synthetic primer
cggcgtgaacaagggcattcctnnsggtgctgccgtc

SEQ ID NO: 198 - DNA, 21 bp, Artificial
  Synthetic primer
aggaatgcccttgttcacgcc

SEQ ID NO: 199 - DNA, 37 bp, Artificial
  Synthetic primer
gaacaagggcattcctgccggtnnsgccgtcgccatt

SEQ ID NO: 200 - DNA, 21 bp, Artificial
  Synthetic primer
accggcaggaatgcccttgtt

SEQ ID NO: 201 - DNA, 45 bp, Artificial
  Synthetic primer
gtgctgccgtcgccattggcnnstatgcagaggatgtgtactaca

SEQ ID NO: 202 - DNA, 25 bp, Artificial
  Synthetic primer
gccaatggcgacggcagcaccggca

SEQ ID NO: 203 - DNA, 45 bp, Artificial
  Synthetic primer
ctgccgtcgccattggccggnnsgcagaggatgtgtactacaacg

SEQ ID NO: 204 - DNA, 25 bp, Artificial
  Synthetic primer
ccggccaatggcgacggcagcaccg

SEQ ID NO: 205 - DNA, 37 bp, Artificial
  Synthetic primer
tgccgtcgccattggccggtatnnsgaggatgtgtac

SEQ ID NO: 206 - DNA, 21 bp, Artificial
  Synthetic primer
ataccggccaatggcgacggc

SEQ ID NO: 207 - DNA, 45 bp, Artificial
  Synthetic primer
ccattggccggtatgcagagnnsgtgtactacaacggcaaccctt

SEQ ID NO: 208 - DNA, 45 bp, Artificial; identical to SEQ ID NO: 207
  Synthetic primer

SEQ ID NO: 209 - DNA, 25 bp, Artificial
  Synthetic primer
ctctgcataccggccaatggcgacg

SEQ ID NO: 210 - DNA, 25 bp, Artificial
  Synthetic primer
ctctgcataccggccaatggcgacg

SEQ ID NO: 211 - DNA, 45 bp, Artificial
  Synthetic primer
ttggccggtatgcagaggatnnstactacaacggcaacccttggt

SEQ ID NO: 212 - DNA, 25 bp, Artificial
  Synthetic primer
atcctctgcataccggccaatggcg

SEQ ID NO: 213 - DNA, 45 bp, Artificial
  Synthetic primer
gccggtatgcagaggatgtgnnstacaacggcaacccttggtatc

SEQ ID NO: 214 - DNA, 25 bp, Artificial
  Synthetic primer
cacatcctctgcataccggccaatg

SEQ ID NO: 215 - DNA, 45 bp, Artificial
  Synthetic primer
ggtatgcagaggatgtgtacnnsaacggcaacccttggtatcttg

SEQ ID NO: 216 - DNA, 27 bp, Artificial
  Synthetic primer
gtacacatcctctgcataccggccaat

SEQ ID NO: 217 - DNA, 45 bp, Artificial
  Synthetic primer
atgcagaggatgtgtactacnnsggcaacccttggtatcttgcta

SEQ ID NO: 218 - DNA, 45 bp, Artificial; identical to SEQ ID NO: 217
  Synthetic primer

SEQ ID NO: 219 - DNA, 26 bp, Artificial
  Synthetic primer
gtagtacacatcctctgcataccggc

SEQ ID NO: 220 - DNA, 26 bp, Artificial
  Synthetic primer
gtagtacacatcctctgcataccggc

SEQ ID NO: 221 - DNA, 45 bp, Artificial
  Synthetic primer
tgtactacaacggcaaccctnnstatcttgctacatttgctgctg

SEQ ID NO: 222 - DNA, 45 bp, Artificial; identical to SEQ ID NO: 221
  Synthetic primer

SEQ ID NO: 223 - DNA, 25 bp, Artificial
  Synthetic primer
agggttgccgttgtagtacacatcc

SEQ ID NO: 224 - DNA, 25 bp, Artificial
  Synthetic primer
agggttgccgttgtagtacacatcc

SEQ ID NO: 225 - DNA, 37 bp, Artificial
  Synthetic primer
gcagctgtacgatgccatctacnnstggaagaagacg

SEQ ID NO: 226 - DNA, 21 bp, Artificial
  Synthetic primer
gtagatggcatcgtacagctg

SEQ ID NO: 227 - DNA, 45 bp, Artificial
  Synthetic primer
acgatgccatctacgtctggnnsaagacgggctccatcacggtga

SEQ ID NO: 228 - DNA, 25 bp, Artificial
  Synthetic primer
ccagacgtagatggcatcgtacagc

SEQ ID NO: 229 - DNA, 45 bp, Artificial
  Synthetic primer
atgccatctacgtctggaagnnsacgggctccatcacggtgaccg

SEQ ID NO: 230 - DNA, 28 bp, Artificial
  Synthetic primer
cttccagacgtagatggcatcgtacagc

SEQ ID NO: 231 - DNA, 38 bp, Artificial
  Synthetic primer
atgccatctacgtctggaagaagnnsggctccatcacg

SEQ ID NO: 232 - DNA, 21 bp, Artificial
  Synthetic primer
cttcttccagacgtagatggc

SEQ ID NO: 233 - DNA, 37 bp, Artificial
  Synthetic primer
ctacgtctggaagaagacgggcnnsatcacggtgacc

SEQ ID NO: 234 - DNA, 22 bp, Artificial
  Synthetic primer
gcccgtcttcttccagacgtag

SEQ ID NO: 235 - DNA, 39 bp, Artificial
  Synthetic primer
ctggaagaagacgggctccatcnnsgtgaccgccacctc

SEQ ID NO: 236 - DNA, 21 bp, Artificial
  Synthetic primer
gatggagcccgtcttcttcca

SEQ ID NO: 237 - DNA, 37 bp, Artificial
  Synthetic primer
gacgggctccatcacggtgaccnnsacctccctggcc

SEQ ID NO: 238 - DNA, 21 bp, Artificial
  Synthetic primer
ggtcaccgtgatggagcccgt

SEQ ID NO: 239 - DNA, 45 bp, Artificial
  Synthetic primer
gctccatcacggtgaccgccnnstccctggccttcttccaggagc

SEQ ID NO: 240 - DNA, 25 bp, Artificial
  Synthetic primer
ggcggtcaccgtgatggagcccgtc

SEQ ID NO: 241 - DNA, 45 bp, Artificial
  Synthetic primer
ccacctccctggccttcttcnnsgagcttgttcctggcgtgacgg

SEQ ID NO: 242 - DNA, 25 bp, Artificial
  Synthetic primer
gaagaaggccagggaggtggcggtc

SEQ ID NO: 243 - DNA, 37 bp, Artificial
  Synthetic primer
cctggccttcttccaggagcttnnscctggcgtgacg

SEQ ID NO: 244 - DNA, 21 bp, Artificial
  Synthetic primer
aagctcctggaagaaggccag

SEQ ID NO: 245 - DNA, 37 bp, Artificial
  Synthetic primer
cttcttccaggagcttgttcctnnsgtgacggccggg

SEQ ID NO: 246 - DNA, 21 bp, Artificial
  Synthetic primer
aggaacaagctcctggaagaa

SEQ ID NO: 247 - DNA, 45 bp, Artificial
  Synthetic primer
aggagcttgttcctggcgtgnnsgccgggacctactccagcagct

SEQ ID NO: 248 - DNA, 25 bp, Artificial
  Synthetic primer
cacgccaggaacaagctcctggaag

SEQ ID NO: 249 - DNA, 37 bp, Artificial
  Synthetic primer
ggagcttgttcctggcgtgacgnnsgggacctactcc

SEQ ID NO: 250 - DNA, 21 bp, Artificial
  Synthetic primer
cgtcacgccaggaacaagctc

SEQ ID NO: 251 - DNA, 45 bp, Artificial
  Synthetic primer
gcgtgacggccgggacctacnnsagcagctcttcgacctttacca

SEQ ID NO: 252 - DNA, 25 bp, Artificial
  Synthetic primer
gtaggtcccggccgtcacgccagga

SEQ ID NO: 253 - DNA, 45 bp, Artificial
  Synthetic primer
tgacggccgggacctactccnnsagctcttcgacctttaccaaca

SEQ ID NO: 254 - DNA, 25 bp, Artificial
  Synthetic primer
ggagtaggtcccggccgtcacgcca

SEQ ID NO: 255 - DNA, 38 bp, Artificial
  Synthetic primer
ctccagcagctcttcgacctttnnsaacatcatcaacg

SEQ ID NO: 256 - DNA, 21 bp, Artificial
  Synthetic primer
aaaggtcgaagagctgctgga

SEQ ID NO: 257 - DNA, 45 bp, Artificial
  Synthetic primer
gcagctcttcgacctttaccnnsatcatcaacgccgtctcgacat

SEQ ID NO: 258 - DNA, 25 bp, Artificial
  Synthetic primer
ggtaaaggtcgaagagctgctggag

SEQ ID NO: 259 - DNA, 37 bp, Artificial
  Synthetic primer
ttcgacctttaccaacatcatcnnsgccgtctcgaca

SEQ ID NO: 260 - DNA, 21 bp, Artificial
  Synthetic primer
gatgatgttggtaaaggtcga

SEQ ID NO: 261 - DNA, 37 bp, Artificial
  Synthetic primer
taccaacatcatcaacgccgtcnnsacatacgccgat

SEQ ID NO: 262 - DNA, 21 bp, Artificial
  Synthetic primer
gacggcgttgatgatgttggt

SEQ ID NO: 263 - DNA, 37 bp, Artificial
  Synthetic primer
gacatacgccgatggcttcctcnnsgaggctgccaag

SEQ ID NO: 264 - DNA, 21 bp, Artificial
  Synthetic primer
gaggaagccatcggcgtatgt

SEQ ID NO: 265 - DNA, 37 bp, Artificial
  Synthetic primer
atacgccgatggcttcctcagcnnsgctgccaagtac

SEQ ID NO: 266 - DNA, 21 bp, Artificial
  Synthetic primer
gctgaggaagccatcggcgta

SEQ ID NO: 267 - DNA, 37 bp, Artificial
  Synthetic primer
cgatggcttcctcagcgaggctnnsaagtacgtcccc

SEQ ID NO: 268 - DNA, 21 bp, Artificial
  Synthetic primer
agcctcgctgaggaagccatc

SEQ ID NO: 269 - DNA, 37 bp, Artificial
  Synthetic primer
tggcttcctcagcgaggctgccnnstacgtccccgcc

SEQ ID NO: 270 - DNA, 21 bp, Artificial
  Synthetic primer
ggcagcctcgctgaggaagcc

SEQ ID NO: 271 - DNA, 45 bp, Artificial
  Synthetic primer
tcctcagcgaggctgccaagnnsgtccccgccgacggttcgctgg

SEQ ID NO: 272 - DNA, 25 bp, Artificial
  Synthetic primer
cttggcagcctcgctgaggaagcca

SEQ ID NO: 273 - DNA, 46 bp, Artificial
  Synthetic primer
aggctgccaagtacgtccccnnsgacggttcgctggccgagcagtt

SEQ ID NO: 274 - DNA, 25 bp, Artificial
  Synthetic primer
ggggacgtacttggcagcctcgctg

SEQ ID NO: 275 - DNA, 45 bp, Artificial
  Synthetic primer
agtacgtccccgccgacggtnnsctggccgagcagtttgaccgca

SEQ ID NO: 276 - DNA, 26 bp, Artificial
  Synthetic primer
accgtcggcggggacgtacttggcag

SEQ ID NO: 277 - DNA, 45 bp, Artificial
  Synthetic primer
cgctggccgagcagtttgacnnsaacagcggcactccgctgtctg

SEQ ID NO: 278 - DNA, 25 bp, Artificial
  Synthetic primer
gtcaaactgctcggccagcgaaccg

SEQ ID NO: 279 - DNA, 45 bp, Artificial
  Synthetic primer
tggccgagcagtttgaccgcnnsagcggcactccgctgtctgcgc

SEQ ID NO: 280 - DNA, 25 bp, Artificial
  Synthetic primer
gcggtcaaactgctcggccagcgaa

SEQ ID NO: 281 - DNA, 37 bp, Artificial
  Synthetic primer
ggccgagcagtttgaccgcaacnnsggcactccgctg

SEQ ID NO: 282 - DNA, 21 bp, Artificial
  Synthetic primer
gttgcggtcaaactgctcggc

SEQ ID NO: 283 - DNA, 45 bp, Artificial
  Synthetic primer
agtttgaccgcaacagcggcnnsccgctgtctgcgcttcacctga

SEQ ID NO: 284 - DNA, 25 bp, Artificial
  Synthetic primer
gccgctgttgcggtcaaactgctcg

SEQ ID NO: 285 - DNA, 45 bp, Artificial
  Synthetic primer
gcaacagcggcactccgctgnnsgcgcttcacctgacgtggtcgt

SEQ ID NO: 286 - DNA, 25 bp, Artificial
  Synthetic primer
cagcggagtgccgctgttgcggtca

SEQ ID NO: 287 - DNA, 38 bp, Artificial
  Synthetic primer
cagcggcactccgctgtctgcgnnscacctgacgtggt

SEQ ID NO: 288 - DNA, 21 bp, Artificial
  Synthetic primer
cgcagacagcggagtgccgct

SEQ ID NO: 289 - DNA, 45 bp, Artificial
  Synthetic primer
gcactccgctgtctgcgcttnnsctgacgtggtcgtacgcctcgt

SEQ ID NO: 290 - DNA, 25 bp, Artificial
  Synthetic primer
aagcgcagacagcggagtgccgctg

SEQ ID NO: 291 - DNA, 45 bp, Artificial
  Synthetic primer
tgtctgcgcttcacctgacgnnstcgtacgcctcgttcttgacag

SEQ ID NO: 292 - DNA, 25 bp, Artificial
  Synthetic primer
cgtcaggtgaagcgcagacagcgga

SEQ ID NO: 293 - DNA, 37 bp, Artificial
  Synthetic primer
gtacgcctcgttcttgacagccnnsgcccgtcgggct

SEQ ID NO: 294 - DNA, 21 bp, Artificial
  Synthetic primer
ggctgtcaagaacgaggcgta

SEQ ID NO: 295 - DNA, 37 bp, Artificial
  Synthetic primer
cgcctcgttcttgacagccacgnnscgtcgggctggc

SEQ ID NO: 296 - DNA, 21 bp, Artificial
  Synthetic primer
cgtggctgtcaagaacgaggc

SEQ ID NO: 297 - DNA, 45 bp, Artificial
  Synthetic primer
tcttgacagccacggcccgtnnsgctggcatcgtgcccccctcgt

SEQ ID NO: 298 - DNA, 25 bp, Artificial
  Synthetic primer
acgggccgtggctgtcaagaacgag

SEQ ID NO: 299 - DNA, 45 bp, Artificial
  Synthetic primer
ccacggcccgtcgggctggcnnsgtgcccccctcgtgggccaaca

SEQ ID NO: 300 - DNA, 25 bp, Artificial
  Synthetic primer
gccagcccgacgggccgtggctgtc

SEQ ID NO: 301 - DNA, 37 bp, Artificial
  Synthetic primer
tggcatcgtgcccccctcgtggnnsaacagcagcgct

SEQ ID NO: 302 - DNA, 21 bp, Artificial
  Synthetic primer
ccacgaggggggcacgatgcc

SEQ ID NO: 303 - DNA, 37 bp, Artificial
  Synthetic primer
catcgtgcccccctcgtgggccnnsagcagcgctagc

SEQ ID NO: 304 - DNA, 21 bp, Artificial
  Synthetic primer
ggcccacgaggggggcacgat

SEQ ID NO: 305 - DNA, 37 bp, Artificial
  Synthetic primer
cgtgcccccctcgtgggccaacnnsagcgctagcacg

SEQ ID NO: 306 - DNA, 21 bp, Artificial
  Synthetic primer
gttggcccacgaggggggcac

SEQ ID NO: 307 - DNA, 37 bp, Artificial
  Synthetic primer
gtgggccaacagcagcgctagcnnsatcccctcgacg

SEQ ID NO: 308 - DNA, 45 bp, Artificial
  Synthetic primer
gcagcgctagcacgatccccnnsacgtgctccggcgcgtccgtgg

SEQ ID NO: 309 - DNA, 25 bp, Artificial
  Synthetic primer
ggggatcgtgctagcgctgctgttg

SEQ ID NO: 310 - DNA, 37 bp, Artificial
  Synthetic primer
ctacacgcccctgccctgcgcgnnsccaacctccgtg

SEQ ID NO: 311 - DNA, 21 bp, Artificial
  Synthetic primer
cgcgcagggcaggggcgtgta

SEQ ID NO: 312 - DNA, 37 bp, Artificial
  Synthetic primer
cacgcccctgccctgcgcgaccnnsacctccgtggcc

SEQ ID NO: 313 - DNA, 21 bp, Artificial
  Synthetic primer
ggtcgcgcagggcaggggcgt

SEQ ID NO: 314 - DNA, 37 bp, Artificial
  Synthetic primer
gcccctgccctgcgcgaccccannstccgtggccgtc

SEQ ID NO: 315 - DNA, 21 bp, Artificial
  Synthetic primer
tggggtcgcgcagggcagggg

SEQ ID NO: 316 - DNA, 38 bp, Artificial
  Synthetic primer
cccaacctccgtggccgtcaccnnscacgagctcgtgt

SEQ ID NO: 317 - DNA, 21 bp, Artificial
  Synthetic primer
ggtgacggccacggaggttgg

SEQ ID NO: 318 - DNA, 37 bp, Artificial
  Synthetic primer
aacctccgtggccgtcaccttcnnsgagctcgtgtcg

SEQ ID NO: 319 - DNA, 21 bp, Artificial
  Synthetic primer
gaaggtgacggccacggaggt

SEQ ID NO: 320 - DNA, 39 bp, Artificial
  Synthetic primer
ctccgtggccgtcaccttccacnnsctcgtgtcgacaca

SEQ ID NO: 321 - DNA, 21 bp, Artificial
  Synthetic primer
gtggaaggtgacggccacgga

SEQ ID NO: 322 - DNA, 37 bp, Artificial
  Synthetic primer
cttccacgagctcgtgtcgacannstttggccagacg

SEQ ID NO: 323 - DNA, 21 bp, Artificial
  Synthetic primer
tgtcgacacgagctcgtggaa

SEQ ID NO: 324 - DNA, 37 bp, Artificial
  Synthetic primer
gctcgtgtcgacacagtttggcnnsacggtcaaggtg

SEQ ID NO: 325 - DNA, 21 bp, Artificial
  Synthetic primer
gccaaactgtgtcgacacgag

SEQ ID NO: 326 - DNA, 45 bp, Artificial
  Synthetic primer
cacagtttggccagacggtcnnsgtggcgggcaacgccgcggccc

SEQ ID NO: 327 - DNA, 25 bp, Artificial
  Synthetic primer
gaccgtctggccaaactgtgtcgac

SEQ ID NO: 328 - DNA, 42 bp, Artificial
  Synthetic primer
tggccagacggtcaaggtggcgnnsaacgccgcggccctggg

SEQ ID NO: 329 - DNA, 27 bp, Artificial
  Synthetic primer
cgccaccttgaccgtctggccaaactg

SEQ ID NO: 330 - DNA, 44 bp, Artificial
  Synthetic primer
ccagacggtcaaggtggcgggcnnsgccgcggccctgggcaact

SEQ ID NO: 331 - DNA, 27 bp, Artificial
  Synthetic primer
gcccgccaccttgaccgtctggccaaa

SEQ ID NO: 332 - DNA, 41 bp, Artificial
  Synthetic primer
gacggtcaaggtggcgggcaacnnsgcggccctgggcaact

SEQ ID NO: 333 - DNA, 27 bp, Artificial
  Synthetic primer
gttgcccgccaccttgaccgtctggcc

SEQ ID NO: 334 - DNA, 41 bp, Artificial
  Synthetic primer
ggtcaaggtggcgggcaacgccnnsgccctgggcaactgga

SEQ ID NO: 335 - DNA, 27 bp, Artificial
  Synthetic primer
ggcgttgcccgccaccttgaccgtctg

SEQ ID NO: 336 - DNA, 41 bp, Artificial
  Synthetic primer
caacgccgcggccctgggcaacnnsagcacgagcgccgccg

SEQ ID NO: 337 - DNA, 27 bp, Artificial
  Synthetic primer
gttgcccagggccgcggcgttgcccgc

SEQ ID NO: 338 - DNA, 41 bp, Artificial
  Synthetic primer
cgcggccctgggcaactggagcnnsagcgccgccgtggctc

SEQ ID NO: 339 - DNA, 27 bp, Artificial
  Synthetic primer
gctccagttgcccagggccgcggcgtt

SEQ ID NO: 340 - DNA, 41 bp, Artificial
  Synthetic primer
caactggagcacgagcgccgccnnsgctctggacgccgtca

SEQ ID NO: 341 - DNA, 27 bp, Artificial
  Synthetic primer
ggcggcgctcgtgctccagttgcccag

SEQ ID NO: 342 - DNA, 42 bp, Artificial
  Synthetic primer
gagcacgagcgccgccgtggctnnsgacgccgtcaactatgc

SEQ ID NO: 343 - DNA, 27 bp, Artificial
  Synthetic primer
agccacggcggcgctcgtgctccagtt

SEQ ID NO: 344 - DNA, 41 bp, Artificial
  Synthetic primer
gagcgccgccgtggctctggacnnsgtcaactatgccgata

SEQ ID NO: 345 - DNA, 27 bp, Artificial
  Synthetic primer
gtccagagccacggcggcgctcgtgct

SEQ ID NO: 346 - DNA, 41 bp, Artificial
  Synthetic primer
cgccgccgtggctctggacgccnnsaactatgccgataacc

SEQ ID NO: 347 - DNA, 27 bp, Artificial
  Synthetic primer
ggcgtccagagccacggcggcgctcgt

SEQ ID NO: 348 - DNA, 37 bp, Artificial
  Synthetic primer
cgccgtggctctggacgccgtcnnstatgccgataac

SEQ ID NO: 349 - DNA, 43 bp, Artificial
  Synthetic primer
cgccgtggctctggacgccgtcnnstatgccgataaccacccc

SEQ ID NO: 350 - DNA, 27 bp, Artificial
  Synthetic primer
gacggcgtccagagccacggcggcgct

SEQ ID NO: 351 - DNA, 27 bp, Artificial
  Synthetic primer
gacggcgtccagagccacggcggcgct

SEQ ID NO: 352 - DNA, 41 bp, Artificial
  Synthetic primer
cgtggctctggacgccgtcaacnnsgccgataaccaccccc

SEQ ID NO: 353 - DNA, 28 bp, Artificial
  Synthetic primer
gttgacggcgtccagagccacggcggcg

SEQ ID NO: 354 - DNA, 41 bp, Artificial
  Synthetic primer
ggctctggacgccgtcaactatnnsgataaccaccccctgt

SEQ ID NO: 355 - DNA, 27 bp, Artificial
  Synthetic primer
atagttgacggcgtccagagccacggc

SEQ ID NO: 356 - DNA, 43 bp, Artificial
  Synthetic primer
tctggacgccgtcaactatgccnnsaaccaccccctgtggatt

SEQ ID NO: 357 - DNA, 27 bp, Artificial
  Synthetic primer
ggcatagttgacggcgtccagagccac

SEQ ID NO: 358 - DNA, 43 bp, Artificial
  Synthetic primer
ggacgccgtcaactatgccgatnnscaccccctgtggattggg

SEQ ID NO: 359 - DNA, 27 bp, Artificial
  Synthetic primer
atcggcatagttgacggcgtccagagc

SEQ ID NO: 360 - DNA, 43 bp, Artificial
  Synthetic primer
ctatgccgataaccaccccctgnnsattgggacggtcaacctc

SEQ ID NO: 361 - DNA, 27 bp, Artificial
  Synthetic primer
cagggggtggttatcggcatagttgac

SEQ ID NO: 362 - DNA, 43 bp, Artificial
  Synthetic primer
tgccgataaccaccccctgtggnnsgggacggtcaacctcgag

SEQ ID NO: 363 - DNA, 27 bp, Artificial
  Synthetic primer
ccacagggggtggttatcggcatagtt

SEQ ID NO: 364 - DNA, 43 bp, Artificial
  Synthetic primer
cgataaccaccccctgtggattnnsacggtcaacctcgaggct

SEQ ID NO: 365 - DNA, 27 bp, Artificial
  Synthetic primer
aatccacagggggtggttatcggcata

SEQ ID NO: 366 - DNA, 43 bp, Artificial
  Synthetic primer
ccaccccctgtggattgggacgnnsaacctcgaggctggagac

SEQ ID NO: 367 - DNA, 27 bp, Artificial
  Synthetic primer
cgtcccaatccacagggggtggttatc

SEQ ID NO: 368 - DNA, 43 bp, Artificial
  Synthetic primer
cctgtggattgggacggtcaacnnsgaggctggagacgtcgtg

SEQ ID NO: 369 - DNA, 27 bp, Artificial
  Synthetic primer
gttgaccgtcccaatccacagggggtg

SEQ ID NO: 370 - DNA, 43 bp, Artificial
  Synthetic primer
tggagacgtcgtggagtacaagnnsatcaatgtgggccaagat

SEQ ID NO: 371 - DNA, 27 bp, Artificial
  Synthetic primer
cttgtactccacgacgtctccagcctc

SEQ ID NO: 372 - DNA, 43 bp, Artificial
  Synthetic primer
cgtcgtggagtacaagtacatcnnsgtgggccaagatggctcc

SEQ ID NO: 373 - DNA, 27 bp, Artificial
  Synthetic primer
gatgtacttgtactccacgacgtctcc

SEQ ID NO: 374 - DNA, 43 bp, Artificial
  Synthetic primer
caagtacatcaatgtgggccaannsggctccgtgacctgggag

SEQ ID NO: 375 - DNA, 27 bp, Artificial
  Synthetic primer
ttggcccacattgatgtacttgtactc

SEQ ID NO: 376 - DNA, 43 bp, Artificial
  Synthetic primer
catcaatgtgggccaagatggcnnsgtgacctgggagagtgat

SEQ ID NO: 377 - DNA, 28 bp, Artificial
  Synthetic primer
gccatcttggcccacattgatgtacttg

SEQ ID NO: 378 - DNA, 43 bp, Artificial
  Synthetic primer
cgtgacctgggagagtgatcccnnscacacttacacggttcct

SEQ ID NO: 379 - DNA, 27 bp, Artificial
  Synthetic primer
gggatcactctcccaggtcacggagcc

SEQ ID NO: 380 - DNA, 43 bp, Artificial
  Synthetic primer
ctgggagagtgatcccaaccacnnstacacggttcctgcggtg

SEQ ID NO: 381 - DNA, 27 bp, Artificial
  Synthetic primer
gtggttgggatcactctcccaggtcac

SEQ ID NO: 382 - DNA, 43 bp, Artificial
  Synthetic primer
tcccaaccacacttacacggttnnsgcggtggcttgtgtgacg

SEQ ID NO: 383 - DNA, 27 bp, Artificial
  Synthetic primer
aaccgtgtaagtgtggttgggatcact

SEQ ID NO: 384 - protein, 588 aa, Talaromyces sp.
GSLDSFLATETPIALQGVLNNIGPNGADVAGASAGIVVASPSRSDPDYFYSWTRDAALTAKYLVDAFIAGNKDLEQTIQEYISAQAQVQTISNPSGDLSTGGLGEPKFNVNETAFTGPWGRPQRDGPALRATALIAYANYLIDNGQASTADEIIWPIVQNDLSYVTQYWNSSTFDLWEEVEGSSFFTTAVQHRALVEGNALATRLNHTCPNCVSQAPQVLCFLQSYWTGSYVLANFGGSGRSGKDVNSILGSIHTFDPAGGCDDSTFQPCSARALANHKVVTDSFRSVYAVNSGIAEGSAVAVGRYPEDVYQGGNPWYLATAAAAEQLYDAIYQWNKIGSISITDVSLAFFQDIYPSAAVGTYNSGSSTFNDIISAVQTYADGYLSIIEKYTPSDGSLTEQFSRSDGTPLSASGLTWSYASLLTAAARRQSIVPASWGESSASSVPAVCSATSATGPYSTATNTAWPSSGSGPSTTTSVPCTTPTSVAVTFDEIVSTTYGETIYLAGSIPELGNWSPSSAIPLRADAYTSSNPLWYVTLNLPAGTSFEYKFFKKETDGTIVWEDDPNRSYTVPAYCGQTTAILDDSWQ

SEQ ID NO: 385 - protein, 112 aa, Humicola grisea
CADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 386 - protein, 109 aa, Thermomyces lanuginosus
CTPPSEVTLTFNALVDTAFGQNIYLVGSIPELGSWDPANALLMSAKSWTSGNPVWTLSISLPAGTSFEYKFIRKDDGSSDVVWESDPNRSYNVPKDCGANTATVNSWWR

SEQ ID NO: 387 - protein, 108 aa, Talaromyces emersonii
CTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 388 - protein, 108 aa, Aspergillus niger [sequence omitted]

SEQ ID NO: 389 - protein, 108 aa, Aspergillus awamori
CTTPTAVAVTFDLTATTTYGENIYLVGSISQLGDWDTSDGIALSADKYTSSNPLWYVTVTLPAGESFEYKFIRIESDDSVEWESDPNREYTVPQACGESTATVTDTWR

SEQ ID NO: 390 - protein, 108 aa, Thielavia terrestris
CSTPTAVAVTFNERVTTQWGQTIKVVGDAAALGGWDTSKAVPLSAAGYTASDPLWSGTVDLPAGLAVQYKYINVAADGGVTWEADPNHSFTVPAACGTTAVTRDDTWQ

SEQ ID NO: 391 - DNA, 32 bp, Artificial
  Synthetic primer
gagagagtgcgggcctcttcgctatttctaga

SEQ ID NO: 392 - DNA, 40 bp, Artificial
  Synthetic primer
caaaataaaatcattatttgtctagaaatagcgaagaggc

SEQ ID NO: 393 - DNA, 40 bp, Artificial
  Synthetic primer
caaataatgattttattttgactgatagtgacctgttcgt

SEQ ID NO: 394 - DNA, 40 bp, Artificial
  Synthetic primer
ttgctcatcaatgtgttgcaacgaacaggtcactatcagt

SEQ ID NO: 395 - DNA, 40 bp, Artificial
  Synthetic primer
tgcaacacattgatgagcaatgcttttttataatgccaac

SEQ ID NO: 396 - DNA, 40 bp, Artificial
  Synthetic primer
agcctgcttttttgtacaaagttggcattataaaaaagca

SEQ ID NO: 397 - DNA, 40 bp, Artificial
  Synthetic primer
tttgtacaaaaaagcaggctatgcacgtcctgtcgactgc

SEQ ID NO: 398 - DNA, 40 bp, Artificial
  Synthetic primer
caacggagccgagcagcaccgcagtcgacaggacgtgcat

SEQ ID NO: 399 - DNA, 40 bp, Artificial
  Synthetic primer
ggtgctgctcggctccgttgccgttcaaaaggtcctggga

SEQ ID NO: 400 - DNA, 40 bp, Artificial
  Synthetic primer
agaccgcttgatcctggtcttcccaggaccttttgaacgg

SEQ ID NO: 401 - DNA, 40 bp, Artificial
  Synthetic primer
agaccaggatcaagcggtctgtccgacgtcaccaagaggt

SEQ ID NO: 402 - DNA, 40 bp, Artificial
  Synthetic primer
gctgatgaagtcgtcaacagacctcttggtgacgtcggac

SEQ ID NO: 403 - DNA, 40 bp, Artificial
  Synthetic primer
ctgttgacgacttcatcagcaccgagacgcctattgcact

SEQ ID NO: 404 - DNA, 40 bp, Artificial
  Synthetic primer
cattgcaaagaagattgttcagtgcaataggcgtctcggt

SEQ ID NO: 405 - DNA, 40 bp, Artificial
  Synthetic primer
gaacaatcttctttgcaatgttggtcctgatggatgccgt

SEQ ID NO: 406 - DNA, 40 bp, Artificial
  Synthetic primer
ccagctgatgtgccgaatgcacggcatccatcaggaccaa

SEQ ID NO: 407 - DNA, 40 bp, Artificial
  Synthetic primer
gcattcggcacatcagctggtgcggtgattgcatctccca

SEQ ID NO: 408 - DNA, 40 bp, Artificial
  Synthetic primer
gtagtccgggtcaattgtgctgggagatgcaatcaccgca

SEQ ID NO: 409 - DNA, 40 bp, Artificial
  Synthetic primer
gcacaattgacccggactactattacatgtggacgcgaga

SEQ ID NO: 410 - DNA, 40 bp, Artificial
  Synthetic primer
tcttgaagacaagagcgctatctcgcgtccacatgtaata

SEQ ID NO: 411 - DNA, 40 bp, Artificial
  Synthetic primer
tagcgctcttgtcttcaagaacctcatcgaccgcttcacc

SEQ ID NO: 412 - DNA, 40 bp, Artificial
  Synthetic primer
aggcccgcatcgtacgtttcggtgaagcggtcgatgaggt

SEQ ID NO: 413 - DNA, 40 bp, Artificial
  Synthetic primer
gaaacgtacgatgcgggcctgcagcgccgcatcgagcagt

SEQ ID NO: 414 - DNA, 40 bp, Artificial
  Synthetic primer
agtgacctgggcagtaatgtactgctcgatgcggcgctgc

SEQ ID NO: 415 - DNA, 40 bp, Artificial
  Synthetic primer
acattactgcccaggtcactctccagggcctctctaaccc

SEQ ID NO: 416 - DNA, 40 bp, Artificial
  Synthetic primer
cgtccgcgagggagcccgaggggttagagaggccctggag

SEQ ID NO: 417 - DNA, 40 bp, Artificial
  Synthetic primer
ctcgggctccctcgcggacggctctggtctcggcgagccc

SEQ ID NO: 418 - DNA, 40 bp, Artificial
  Synthetic primer
ttcagggtcaactcaaacttgggctcgccgagaccagagc

SEQ ID NO: 419 - DNA, 40 bp, Artificial
  Synthetic primer
aagtttgagttgaccctgaagcctttcaccggcaactggg

SEQ ID NO: 420 - DNA, 40 bp, Artificial
  Synthetic primer
gccatcccgctgcggtcgaccccagttgccggtgaaaggc

SEQ ID NO: 421 - DNA, 40 bp, Artificial
  Synthetic primer
gtcgaccgcagcgggatggcccagctctgcgagccattgc

SEQ ID NO: 422 - DNA, 40 bp, Artificial
  Synthetic primer
actttgagtatccaatcaaggcaatggctcgcagagctgg

SEQ ID NO: 423 - DNA, 40 bp, Artificial
  Synthetic primer
cttgattggatactcaaagtggctcatcaacaacaactat

SEQ ID NO: 424 - DNA, 40 bp, Artificial
  Synthetic primer
acgttggacacagtcgactgatagttgttgttgatgagcc

SEQ ID NO: 425 - DNA, 40 bp, Artificial
  Synthetic primer
cagtcgactgtgtccaacgtcatctggcctattgtgcgca

SEQ ID NO: 426 - DNA, 40 bp, Artificial
  Synthetic primer
ggcaacatagttgaggtcgttgcgcacaataggccagatg

SEQ ID NO: 427 - DNA, 40 bp, Artificial
  Synthetic primer
acgacctcaactatgttgcccagtactggaaccaaaccgg

SEQ ID NO: 428 - DNA, 40 bp, Artificial
  Synthetic primer
cttcttcccagaggtcaaagccggtttggttccagtactg

SEQ ID NO: 429 - DNA, 40 bp, Artificial
  Synthetic primer
ctttgacctctgggaagaagtcaatgggagctcattcttt

SEQ ID NO: 430 - DNA, 40 bp, Artificial
  Synthetic primer
cggtgctggttggcaacagtaaagaatgagctcccattga

SEQ ID NO: 431 - DNA, 40 bp, Artificial
  Synthetic primer
actgttgccaaccagcaccgagcacttgtcgagggcgcca

SEQ ID NO: 432 - DNA, 40 bp, Artificial
  Synthetic primer
gccaagagtggcagcaagagtggcgccctcgacaagtgct

SEQ ID NO: 433 - DNA, 40 bp, Artificial
  Synthetic primer
ctcttgctgccactcttggccagtcgggaagcgcttattc

SEQ ID NO: 434 - DNA, 40 bp, Artificial
  Synthetic primer
aaacctggggagcaacagatgaataagcgcttcccgactg

SEQ ID NO: 435 - DNA, 40 bp, Artificial
  Synthetic primer
atctgttgctccccaggttttgtgctttctccaacgattc

SEQ ID NO: 436 - DNA, 40 bp, Artificial
  Synthetic primer
tatccaccagacgacacccagaatcgttggagaaagcaca

SEQ ID NO: 437 - DNA, 40 bp, Artificial
  Synthetic primer
tgggtgtcgtctggtggatacgtcgactccaacatcaaca

SEQ ID NO: 438 - DNA, 40 bp, Artificial
  Synthetic primer
gccagtcctgccctcgttggtgttgatgttggagtcgacg

SEQ ID NO: 439 - DNA, 40 bp, Artificial
  Synthetic primer
ccaacgagggcaggactggcaaggatgtcaactccgtcct

SEQ ID NO: 440 - DNA, 40 bp, Artificial
  Synthetic primer
cgaaggtgtggatggaagtcaggacggagttgacatcctt

SEQ ID NO: 441 - DNA, 40 bp, Artificial
  Synthetic primer
gacttccatccacaccttcgatcccaaccttggctgtgac

SEQ ID NO: 442 - DNA, 40 bp, Artificial
  Synthetic primer
catggctggaaggtgcctgcgtcacagccaaggttgggat

SEQ ID NO: 443 - DNA, 40 bp, Artificial
  Synthetic primer
gcaggcaccttccagccatgcagtgacaaagcgctctcca

SEQ ID NO: 444 - DNA, 40 bp, Artificial
  Synthetic primer
gtcgacaacaaccttgaggttggagagcgctttgtcactg

SEQ ID NO: 445 - DNA, 40 bp, Artificial
  Synthetic primer
acctcaaggttgttgtcgactccttccgctccatctacgg

SEQ ID NO: 446 - DNA, 40 bp, Artificial
  Synthetic primer
caggaatgcccttgttcacgccgtagatggagcggaagga

SEQ ID NO: 447 - DNA, 40 bp, Artificial
  Synthetic primer
cgtgaacaagggcattcctgccggtgctgccgtcgccatt

SEQ ID NO: 448 - DNA, 40 bp, Artificial
  Synthetic primer
acatcctctgcataccggccaatggcgacggcagcaccgg

SEQ ID NO: 449 - DNA, 40 bp, Artificial
  Synthetic primer
ggccggtatgcagaggatgtgtactacaacggcaaccctt

SEQ ID NO: 450 - DNA, 40 bp, Artificial
  Synthetic primer
agcaaatgtagcaagataccaagggttgccgttgtagtac

SEQ ID NO: 451 - DNA, 40 bp, Artificial
  Synthetic primer
ggtatcttgctacatttgctgctgccgagcagctgtacga

SEQ ID NO: 452 - DNA, 40 bp, Artificial
  Synthetic primer
tcttccagacgtagatggcatcgtacagctgctcggcagc

SEQ ID NO: 453 - DNA, 40 bp, Artificial
  Synthetic primer
tgccatctacgtctggaagaagacgggctccatcacggtg

SEQ ID NO: 454 - DNA, 40 bp, Artificial
  Synthetic primer
aaggccagggaggtggcggtcaccgtgatggagcccgtct

SEQ ID NO: 455 - DNA, 40 bp, Artificial
  Synthetic primer
accgccacctccctggccttcttccaggagcttgttcctg

SEQ ID NO: 456 - DNA, 40 bp, Artificial
  Synthetic primer
gtaggtcccggccgtcacgccaggaacaagctcctggaag

SEQ ID NO: 457 - DNA, 40 bp, Artificial
  Synthetic primer
gcgtgacggccgggacctactccagcagctcttcgacctt

SEQ ID NO: 458 - DNA, 40 bp, Artificial
  Synthetic primer
cggcgttgatgatgttggtaaaggtcgaagagctgctgga

SEQ ID NO: 459 - DNA, 40 bp, Artificial
  Synthetic primer
taccaacatcatcaacgccgtctcgacatacgccgatggc

SEQ ID NO: 460 - DNA, 40 bp, Artificial
  Synthetic primer
ttggcagcctcgctgaggaagccatcggcgtatgtcgaga

SEQ ID NO: 461 - DNA, 40 bp, Artificial
  Synthetic primer
ttcctcagcgaggctgccaagtacgtccccgccgacggtt

SEQ ID NO: 462 - DNA, 40 bp, Artificial
  Synthetic primer
gtcaaactgctcggccagcgaaccgtcggcggggacgtac

SEQ ID NO: 463 - DNA, 40 bp, Artificial
  Synthetic primer
cgctggccgagcagtttgaccgcaacagcggcactccgct

SEQ ID NO: 464 - DNA, 40 bp, Artificial
  Synthetic primer
acgtcaggtgaaccgcagacagcggagtgccgctgttgcg

SEQ ID NO: 465 - DNA, 40 bp, Artificial
  Synthetic primer
gtctgcggttcacctgacgtggtcgtacgcctcgttcttg

SEQ ID NO: 466 - DNA, 40 bp, Artificial
  Synthetic primer
gcccgacgaagcgtggctgtcaagaacgaggcgtacgacc

SEQ ID NO: 467 - DNA, 40 bp, Artificial
  Synthetic primer
acagccacgcttcgtcgggctggcatcgtgcccccctcgt

SEQ ID NO: 468 - DNA, 40 bp, Artificial
  Synthetic primer
gctagcgctgctgttggcccacgaggggggcacgatgcca

SEQ ID NO: 469 - DNA, 40 bp, Artificial
  Synthetic primer
gggccaacagcagcgctagcacgatcccctcgacgtgctc

SEQ ID NO: 470 - DNA, 40 bp, Artificial
  Synthetic primer
atccgaccacggacgcgccggagcacgtcgaggggatcgt

SEQ ID NO: 471 - DNA, 40 bp, Artificial
  Synthetic primer
cggcgcgtccgtggtcggatcctactcgcgtcccaccgcc

SEQ ID NO: 472 - DNA, 40 bp, Artificial
  Synthetic primer
tgcgacggagggaatgacgtggcggtgggacgcgagtagg

SEQ ID NO: 473 - DNA, 40 bp, Artificial
  Synthetic primer
acgtcattccctccgtcgcagacgcccaagcctggcgtgc

SEQ ID NO: 474 - DNA, 40 bp, Artificial
  Synthetic primer
cgtgtagggagtaccggaaggcacgccaggcttgggcgtc

SEQ ID NO: 475 - DNA, 40 bp, Artificial
  Synthetic primer
cttccggtactccctacacgcccctgccctgcgcgacccc

SEQ ID NO: 476 - DNA, 40 bp, Artificial
  Synthetic primer
aggtgacggccacggaggttggggtcgcgcagggcagggg

SEQ ID NO: 477 - DNA, 40 bp, Artificial
  Synthetic primer
aacctccgtggccgtcaccttccacgagctcgtgtcgaca

SEQ ID NO: 478 - DNA, 40 bp, Artificial
  Synthetic primer
ttgaccgtctggccaaactgtgtcgacacgagctcgtgga

SEQ ID NO: 479 - DNA, 40 bp, Artificial
  Synthetic primer
cagtttggccagacggtcaaggtggcgggcaacgccgcgg

SEQ ID NO: 480 - DNA, 40 bp, Artificial
  Synthetic primer
cgtgctccagttgcccagggccgcggcgttgcccgccacc

SEQ ID NO: 481 - DNA, 40 bp, Artificial
  Synthetic primer
ccctgggcaactggagcacgagcgccgccgtggctctgga

SEQ ID NO: 482 - DNA, 40 bp, Artificial
  Synthetic primer
tatcacgatagttgacggcgtccagagccacggcggcgct

SEQ ID NO: 483 - DNA, 40 bp, Artificial
  Synthetic primer
cgccgtcaactatcgtgataaccaccccctgtggattggg

SEQ ID NO: 484 - DNA, 40 bp, Artificial
  Synthetic primer
ccagcctcgaggttgaccgtcccaatccacagggggtggt

SEQ ID NO: 485 - DNA, 40 bp, Artificial
  Synthetic primer
acggtcaacctcgaggctggagacgtcgtggagtacaagt

SEQ ID NO: 486 - DNA, 40 bp, Artificial
  Synthetic primer
atcttggcccacattgatgtacttgtactccacgacgtct

SEQ ID NO: 487 - DNA, 40 bp, Artificial
  Synthetic primer
acatcaatgtgggccaagatggctccgtgacctgggagag

SEQ ID NO: 488 - DNA, 40 bp, Artificial
  Synthetic primer
tgtaagtgtggttgggatcactctcccaggtcacggagcc

SEQ ID NO: 489 - DNA, 40 bp, Artificial
  Synthetic primer
tgatcccaaccacacttacacggttcctgcggtggcttgt

SEQ ID NO: 490 - DNA, 40 bp, Artificial
  Synthetic primer
tccttgacaacctgcgtcacacaagccaccgcaggaaccg

SEQ ID NO: 491 - DNA, 40 bp, Artificial
  Synthetic primer
gtgacgcaggttgtcaaggaggacacctggcagtcgtaaa

SEQ ID NO: 492 - DNA, 40 bp, Artificial
  Synthetic primer
ctttgtacaagaaagctgggtttacgactgccaggtgtcc

SEQ ID NO: 493 - DNA, 40 bp, Artificial
  Synthetic primer
cccagctttcttgtacaaagttggcattataagaaagcat

SEQ ID NO: 494 - DNA, 40 bp, Artificial
  Synthetic primer
ttgcaacaaattgataagcaatgctttcttataatgccaa

SEQ ID NO: 495 - DNA, 40 bp, Artificial
  Synthetic primer
tgcttatcaatttgttgcaacgaacaggtcactatcagtc

SEQ ID NO: 496 - DNA, 40 bp, Artificial
  Synthetic primer
tcaaataatgattttattttgactgatagtgacctgttcg

SEQ ID NO: 497 - DNA, 40 bp, Artificial
  Synthetic primer
aaaataaaatcattatttgaagcttaagcctggggtgcct

SEQ ID NO: 498 - DNA, 31 bp, Artificial
  Synthetic primer
agagagtcattaggcaccccaggcttaagct

SEQ ID NO: 499 - DNA, 40 bp, Artificial
  Synthetic primer
gtagtccgggtcttgtgtgctgggagatgcaatcaccgca

SEQ ID NO: 500 - DNA, 40 bp, Artificial
  Synthetic primer
gcacacaagacccggactactattacatgtggacgcgaga

SEQ ID NO: 501 - DNA, 40 bp, Artificial
  Synthetic primer
tagcgctcttgtcttcaagattctcatcgaccgcttcacc

SEQ ID NO: 502 - DNA, 40 bp, Artificial
  Synthetic primer
aggcccgcatcgtacgtttcggtgaagcggtcgatgagaa

SEQ ID NO: 503 - DNA, 40 bp, Artificial
  Synthetic primer
tatcacgatagttgacacggtccagagccacggcggcgct

SEQ ID NO: 504 - DNA, 40 bp, Artificial
  Synthetic primer
ccgtgtcaactatcgtgataaccaccccctgtggattggg

SEQ ID NO: 505 - DNA, 40 bp, Artificial; identical to SEQ ID NO: 499
  Synthetic primer

SEQ ID NO: 506 - DNA, 40 bp, Artificial; identical to SEQ ID NO: 500
  Synthetic primer

SEQ ID NO: 507 - DNA, 40 bp, Artificial; identical to SEQ ID NO: 501
  Synthetic primer

SEQ ID NO: 508 - DNA, 40 bp, Artificial; identical to SEQ ID NO: 502
  Synthetic primer

SEQ ID NO: 509 - DNA, 40 bp, Artificial; identical to SEQ ID NO: 501
  Synthetic primer

SEQ ID NO: 510 - DNA, 40 bp, Artificial; identical to SEQ ID NO: 502
  Synthetic primer

SEQ ID NO: 511 - DNA, 40 bp, Artificial; identical to SEQ ID NO: 503
  Synthetic primer

SEQ ID NO: 512 - DNA, 40 bp, Artificial; identical to SEQ ID NO: 504
  Synthetic primer

SEQ ID NO: 513 - DNA, 40 bp, Artificial; identical to SEQ ID NO: 503
  Synthetic primer

SEQ ID NO: 514 - DNA, 40 bp, Artificial; identical to SEQ ID NO: 504
  Synthetic primer

SEQ ID NO: 515 - DNA, 40 bp, Artificial; identical to SEQ ID NO: 499
  Synthetic primer

SEQ ID NO: 516 - DNA, 40 bp, Artificial; identical to SEQ ID NO: 500
  Synthetic primer

SEQ ID NO: 517 - DNA, 40 bp, Artificial; identical to SEQ ID NO: 499
  Synthetic primer

SEQ ID NO: 518 - DNA, 40 bp, Artificial; identical to SEQ ID NO: 500
  Synthetic primer

SEQ ID NO: 519 - DNA, 40 bp, Artificial; identical to SEQ ID NO: 501
  Synthetic primer

SEQ ID NO: 520 - DNA, 40 bp, Artificial; identical to SEQ ID NO: 502
  Synthetic primer

SEQ ID NO: 521 - DNA, 40 bp, Artificial; identical to SEQ ID NO: 503
  Synthetic primer

SEQ ID NO: 522 - DNA, 40 bp, Artificial; identical to SEQ ID NO: 504
  Synthetic primer

SEQ ID NO: 523 - DNA, 40 bp, Artificial; identical to SEQ ID NO: 499
  Synthetic primer

SEQ ID NO: 524 - DNA, 40 bp, Artificial; identical to SEQ ID NO: 500
  Synthetic primer

SEQ ID NO: 525 - DNA, 40 bp, Artificial; identical to SEQ ID NO: 501
  Synthetic primer

SEQ ID NO: 526 - DNA, 40 bp, Artificial; identical to SEQ ID NO: 502
  Synthetic primer

SEQ ID NO: 527 - DNA, 40 bp, Artificial; identical to SEQ ID NO: 503
  Synthetic primer

SEQ ID NO: 528 - DNA, 40 bp, Artificial; identical to SEQ ID NO: 504
  Synthetic primer

SEQ ID NO: 529 - DNA, 40 bp, Artificial
  Synthetic primer
gcccgacgaagagcggctgtcaagaacgaggcgtacgacc

SEQ ID NO: 530 - DNA, 40 bp, Artificial
  Synthetic primer
acagccgctcttcgtcgggctggcatcgtgcccccctcgt

SEQ ID NO: 531 - DNA, 40 bp, Artificial; identical to SEQ ID NO: 529
  Synthetic primer

SEQ ID NO: 532 - DNA, 40 bp, Artificial; identical to SEQ ID NO: 530
  Synthetic primer

SEQ ID NO: 533 - DNA, 40 bp, Artificial
  Synthetic primer
ttgaccgtatggccaaactgtgtcgacacgagctcgtgga

SEQ ID NO: 534 - DNA, 40 bp, Artificial
  Synthetic primer
cagtttggccatacggtcaaggtggcgggcaacgccgcgg

SEQ ID NO: 535 - DNA, 40 bp, Artificial; identical to SEQ ID NO: 503
  Synthetic primer

SEQ ID NO: 536 - DNA, 40 bp, Artificial; identical to SEQ ID NO: 504
  Synthetic primer

SEQ ID NO: 537 - DNA, 40 bp, Artificial
  Synthetic primer
atcttggcccacaatgatgtacttgtactccacgacgtct

SEQ ID NO: 538 - DNA, 40 bp, Artificial
  Synthetic primer
acatcattgtgggccaagatggctccgtgacctgggagag

SEQ ID NO: 539 - DNA, 40 bp, Artificial; identical to SEQ ID NO: 499
  Synthetic primer

SEQ ID NO: 540 - DNA, 40 bp, Artificial; identical to SEQ ID NO: 500
  Synthetic primer

SEQ ID NO: 541 - DNA, 40 bp, Artificial; identical to SEQ ID NO: 529
  Synthetic primer

SEQ ID NO: 542 - DNA, 40 bp, Artificial; identical to SEQ ID NO: 530
  Synthetic primer

SEQ ID NO: 543 - DNA, 40 bp, Artificial; identical to SEQ ID NO: 533
  Synthetic primer

SEQ ID NO: 544 - DNA, 40 bp, Artificial; identical to SEQ ID NO: 534
  Synthetic primer

SEQ ID NO: 545 - DNA, 40 bp, Artificial; identical to SEQ ID NO: 503
  Synthetic primer

SEQ ID NO: 546 - DNA, 40 bp, Artificial; identical to SEQ ID NO: 504
  Synthetic primer

SEQ ID NO: 547 - DNA, 40 bp, Artificial; identical to SEQ ID NO: 537
  Synthetic primer

SEQ ID NO: 548 - DNA, 40 bp, Artificial; identical to SEQ ID NO: 538
  Synthetic primer

SEQ ID NO: 549 - DNA, 40 bp, Artificial; identical to SEQ ID NO: 501
  Synthetic primer

SEQ ID NO: 550 - DNA, 40 bp, Artificial; identical to SEQ ID NO: 502
  Synthetic primer

SEQ ID NO: 551 - DNA, 40 bp, Artificial; identical to SEQ ID NO: 529
  Synthetic primer

SEQ ID NO: 552 - DNA, 40 bp, Artificial; identical to SEQ ID NO: 530
  Synthetic primer

SEQ ID NO: 553 - DNA, 40 bp, Artificial; identical to SEQ ID NO: 533
  Synthetic primer

SEQ ID NO: 554 - DNA, 40 bp, Artificial; identical to SEQ ID NO: 534
  Synthetic primer

SEQ ID NO: 555 - DNA, 40 bp, Artificial; identical to SEQ ID NO: 503
  Synthetic primer

SEQ ID NO: 556 - DNA, 40 bp, Artificial; identical to SEQ ID NO: 504
  Synthetic primer

SEQ ID NO: 557 - DNA, 40 bp, Artificial; identical to SEQ ID NO: 537
  Synthetic primer

SEQ ID NO: 558 - DNA, 40 bp, Artificial; identical to SEQ ID NO: 538
  Synthetic primer

SEQ ID NO: 559 - DNA, 40 bp, Artificial; identical to SEQ ID NO: 499
  Synthetic primer

SEQ ID NO: 560 - DNA, 40 bp, Artificial; identical to SEQ ID NO: 500
  Synthetic primer

SEQ ID NO: 561 - DNA, 40 bp, Artificial; identical to SEQ ID NO: 501
  Synthetic primer

SEQ ID NO: 562 - DNA, 40 bp, Artificial; identical to SEQ ID NO: 502
  Synthetic primer

SEQ ID NO: 563 - DNA, 40 bp, Artificial
  Synthetic primer
gtagtccgggtcacgtgtgctgggagatgcaatcaccgca

SEQ ID NO: 564 - DNA, 40 bp, Artificial
  Synthetic primer
gcacacgtgacccggactactattacatgtggacgcgaga

SEQ ID NO: 565 - DNA, 40 bp, Artificial; identical to SEQ ID NO: 563
  Synthetic primer

SEQ ID NO: 566 - DNA, 40 bp, Artificial; identical to SEQ ID NO: 564
  Synthetic primer

SEQ ID NO: 567 - DNA, 40 bp, Artificial; identical to SEQ ID NO: 501
  Synthetic primer

SEQ ID NO: 568 - DNA, 40 bp, Artificial; identical to SEQ ID NO: 502
  Synthetic primer

SEQ ID NO: 569 - DNA, 40 bp, Artificial; identical to SEQ ID NO: 563
  Synthetic primer

SEQ ID NO: 570 - DNA, 40 bp, Artificial; identical to SEQ ID NO: 564
  Synthetic primer

SEQ ID NO: 571 - DNA, 40 bp, Artificial; identical to SEQ ID NO: 501
  Synthetic primer

SEQ ID NO: 572 - DNA, 40 bp, Artificial; identical to SEQ ID NO: 502
  Synthetic primer

SEQ ID NO: 573 - DNA, 40 bp, Artificial; identical to SEQ ID NO: 503
  Synthetic primer

SEQ ID NO: 574 - DNA, 40 bp, Artificial; identical to SEQ ID NO: 504
  Synthetic primer

SEQ ID NO: 575 - DNA, 40 bp, Artificial; identical to SEQ ID NO: 529
  Synthetic primer

SEQ ID NO: 576 - DNA, 40 bp, Artificial; identical to SEQ ID NO: 530
  Synthetic primer

SEQ ID NO: 577 - DNA, 40 bp, Artificial
  Synthetic primer
acgtcaggtgacgcgcagacagcggagtgccgctgttgcg

SEQ ID NO: 578 - DNA, 40 bp, Artificial
  Synthetic primer
gtctgcgcgtcacctgacgtggtcgtacgcctcgttcttg

SEQ ID NO: 579 - DNA, 40 bp, Artificial
  Synthetic primer
acgtcaggtgacccgcagacagcggagtgccgctgttgcg

SEQ ID NO: 580 - DNA, 40 bp, Artificial
  Synthetic primer
gtctgcgggtcacctgacgtggtcgtacgcctcgttcttg

SEQ ID NO: 581 - DNA, 40 bp, Artificial
  Synthetic primer
agaaacgcatcgtacgtttcggtgaagcggtcgatgaggt

SEQ ID NO: 582 - DNA, 40 bp, Artificial
  Synthetic primer
gaaacgtacgatgcgtttctgcagcgccgcatcgagcagt

SEQ ID NO: 583 - DNA, 40 bp, Artificial; identical to SEQ ID NO: 577
  Synthetic primer

SEQ ID NO: 584 - DNA, 40 bp, Artificial; identical to SEQ ID NO: 578
  Synthetic primer

SEQ ID NO: 585 - DNA, 40 bp, Artificial
  Synthetic primer
gcccgacgggccgtggctgtcaagaacgaggcgtacgacc

SEQ ID NO: 586 - DNA, 40 bp, Artificial
  Synthetic primer
acagccacggcccgtcgggctggcatcgtgcccccctcgt

SEQ ID NO: 587 - DNA, 40 bp, Artificial
  Synthetic primer
aacctccgtggccgtcaccttccacgttctcgtgtcgaca

SEQ ID NO: 588 - DNA, 40 bp, Artificial
  Synthetic primer
ttgaccgtctggccaaactgtgtcgacacgagaacgtgga

SEQ ID NO: 589 - DNA, 40 bp, Artificial
  Synthetic primer
atcttggcccactttgatgtacttgtactccacgacgtct

SEQ ID NO: 590 - DNA, 40 bp, Artificial
  Synthetic primer
acatcaaagtgggccaagatggctccgtgacctgggagag

SEQ ID NO: 591 - DNA, 40 bp, Artificial; identical to SEQ ID NO: 563
  Synthetic primer

SEQ ID NO: 592 - DNA, 40 bp, Artificial; identical to SEQ ID NO: 564
  Synthetic primer

SEQ ID NO: 593 - DNA, 40 bp, Artificial; identical to SEQ ID NO: 581
  Synthetic primer

SEQ ID NO: 594 - DNA, 40 bp, Artificial; identical to SEQ ID NO: 582
  Synthetic primer

SEQ ID NO: 595 - DNA, 40 bp, Artificial; identical to SEQ ID NO: 577
  Synthetic primer

SEQ ID NO: 596 - DNA, 40 bp, Artificial; identical to SEQ ID NO: 578
  Synthetic primer

SEQ ID NO: 597 - DNA, 40 bp, Artificial; identical to SEQ ID NO: 585
  Synthetic primer

SEQ ID NO: 598 - DNA, 40 bp, Artificial; identical to SEQ ID NO: 586
  Synthetic primer

SEQ ID NO: 599 - DNA, 40 bp, Artificial; identical to SEQ ID NO: 587
  Synthetic primer

SEQ ID NO: 600 - DNA, 40 bp, Artificial; identical to SEQ ID NO: 588
  Synthetic primer

SEQ ID NO: 601 - DNA, 40 bp, Artificial; identical to SEQ ID NO: 589
  Synthetic primer

SEQ ID NO: 602 - DNA, 40 bp, Artificial; identical to SEQ ID NO: 590
  Synthetic primer

SEQ ID NO: 603 - DNA, 40 bp, Artificial; identical to SEQ ID NO: 563
  Synthetic primer

SEQ ID NO: 604 - DNA, 40 bp, Artificial; identical to SEQ ID NO: 564
  Synthetic primer

SEQ ID NO: 605 - DNA, 40 bp, Artificial; identical to SEQ ID NO: 581
  Synthetic primer

SEQ ID NO: 606 - DNA, 40 bp, Artificial; identical to SEQ ID NO: 582
  Synthetic primer

SEQ ID NO: 607 - DNA, 40 bp, Artificial
  Synthetic primer
acgtcaggtgaagcgcagacagcggagtgccgctgttgcg

SEQ ID NO: 608 - DNA, 40 bp, Artificial
  Synthetic primer
gtctgcgcttcacctgacgtggtcgtacgcctcgttcttg

SEQ ID NO: 609 - DNA, 40 bp, Artificial; identical to SEQ ID NO: 585
  Synthetic primer

SEQ ID NO: 610 - DNA, 40 bp, Artificial; identical to SEQ ID NO: 586
  Synthetic primer

SEQ ID NO: 611 - DNA, 40 bp, Artificial; identical to SEQ ID NO: 587
  Synthetic primer

SEQ ID NO: 612 - DNA, 40 bp, Artificial
  Synthetic primer
ttgaccgtatggccaaactgtgtcgacacgagaacgtgga

SEQ ID NO: 613 - DNA, 40 bp, Artificial; identical to SEQ ID NO: 534
  Synthetic primer

SEQ ID NO: 614 - DNA, 40 bp, Artificial
  Synthetic primer
tatcggcatagttgacggcgtccagagccacggcggcgct

SEQ ID NO: 615 - DNA, 40 bp, Artificial
  Synthetic primer
cgccgtcaactatgccgataaccaccccctgtggattggg

SEQ ID NO: 616 - DNA, 40 bp, Artificial; identical to SEQ ID NO: 589
  Synthetic primer

SEQ ID NO: 617 - DNA, 40 bp, Artificial; identical to SEQ ID NO: 590
  Synthetic primer

SEQ ID NO: 618 - DNA, 25 bp, Artificial
  Synthetic primer
acaagtttgtacaaaaaagcaggct

SEQ ID NO: 619 - DNA, 40 bp, Artificial
  Synthetic primer
gcagtcgacaggacgtgcatagcctgcttttttgtacaaa

SEQ ID NO: 620 - DNA, 40 bp, Artificial
  Synthetic primer
atgcacgtcctgtcgactgcggtgctgctcggctccgttg

SEQ ID NO: 621 - DNA, 40 bp, Artificial
  Synthetic primer
tcccaggaccttttgaacggcaacggagccgagcagcacc

SEQ ID NO: 622 - DNA, 40 bp, Artificial
  Synthetic primer
ccgttcaaaaggtcctgggaagaccaggatcaagcggtct

SEQ ID NO: 623 - DNA, 40 bp, Artificial
  Synthetic primer
acctcttggtgacgtcggacagaccgcttgatcctggtct

SEQ ID NO: 624 - DNA, 40 bp, Artificial
  Synthetic primer
gtccgacgtcaccaagaggtctgttgacgacttcatcagc

SEQ ID NO: 625 - DNA, 40 bp, Artificial
  Synthetic primer
agtgcaataggcgtctcggtgctgatgaagtcgtcaacag

SEQ ID NO: 626 - DNA, 40 bp, Artificial
  Synthetic primer
accgagacgcctattgcactgaacaatcttctttgcaatg

SEQ ID NO: 627 - DNA, 40 bp, Artificial
  Synthetic primer
acggcatccatcaggaccaacattgcaaagaagattgttc

SEQ ID NO: 628 - DNA, 40 bp, Artificial
  Synthetic primer
ttggtcctgatggatgccgtgcattcggcacatcagctgg

SEQ ID NO: 629 - DNA, 40 bp, Artificial
  Synthetic primer
tgggagatgcaatcaccgcaccagctgatgtgccgaatgc

SEQ ID NO: 630 - DNA, 40 bp, Artificial
  Synthetic primer
tgcggtgattgcatctcccagcacaattgacccggactac

SEQ ID NO: 631 - DNA, 40 bp, Artificial
  Synthetic primer
tctcgcgtccacatgtaatagtagtccgggtcaattgtgc

SEQ ID NO: 632 - DNA, 40 bp, Artificial
  Synthetic primer
tattacatgtggacgcgagatagcgctcttgtcttcaaga

SEQ ID NO: 633 - DNA, 40 bp, Artificial
  Synthetic primer
ggtgaagcggtcgatgaggttcttgaagacaagagcgcta

SEQ ID NO: 634 - DNA, 40 bp, Artificial
  Synthetic primer
acctcatcgaccgcttcaccgaaacgtacgatgcgggcct

SEQ ID NO: 635 - DNA, 40 bp, Artificial
  Synthetic primer
actgctcgatgcggcgctgcaggcccgcatcgtacgtttc

SEQ ID NO: 636 - DNA, 40 bp, Artificial
  Synthetic primer
gcagcgccgcatcgagcagtacattactgcccaggtcact

SEQ ID NO: 637 - DNA, 40 bp, Artificial
  Synthetic primer
gggttagagaggccctggagagtgacctgggcagtaatgt

SEQ ID NO: 638 - DNA, 40 bp, Artificial
  Synthetic primer
ctccagggcctctctaacccctcgggctccctcgcggacg

SEQ ID NO: 639 - DNA, 40 bp, Artificial
  Synthetic primer
gggctcgccgagaccagagccgtccgcgagggagcccgag

SEQ ID NO: 640 - DNA, 40 bp, Artificial
  Synthetic primer
gctctggtctcggcgagcccaagtttgagttgaccctgaa

SEQ ID NO: 641 - DNA, 40 bp, Artificial
  Synthetic primer
cccagttgccggtgaaaggcttcagggtcaactcaaactt

SEQ ID NO: 642 - DNA, 40 bp, Artificial
  Synthetic primer
gcctttcaccggcaactggggtcgaccgcagcgggatggc

SEQ ID NO: 643 - DNA, 40 bp, Artificial
  Synthetic primer
gcaatggctcgcagagctgggccatcccgctgcggtcgac

SEQ ID NO: 644 - DNA, 40 bp, Artificial
  Synthetic primer
ccagctctgcgagccattgccttgattggatactcaaagt

SEQ ID NO: 645 - DNA, 40 bp, Artificial
  Synthetic primer
atagttgttgttgatgagccactttgagtatccaatcaag

SEQ ID NO: 646 - DNA, 40 bp, Artificial
  Synthetic primer
ggctcatcaacaacaactatcagtcgactgtgtccaacgt

SEQ ID NO: 647 - DNA, 40 bp, Artificial
  Synthetic primer
tgcgcacaataggccagatgacgttggacacagtcgactg

SEQ ID NO: 648 - DNA, 40 bp, Artificial
  Synthetic primer
catctggcctattgtgcgcaacgacctcaactatgttgcc

SEQ ID NO: 649 - DNA, 40 bp, Artificial
  Synthetic primer
ccggtttggttccagtactgggcaacatagttgaggtcgt

SEQ ID NO: 650 - DNA, 40 bp, Artificial
  Synthetic primer
cagtactggaaccaaaccggctttgacctctgggaagaag

SEQ ID NO: 651 - DNA, 40 bp, Artificial
  Synthetic primer
aaagaatgagctcccattgacttcttcccagaggtcaaag

SEQ ID NO: 652 - DNA, 40 bp, Artificial
  Synthetic primer
tcaatgggagctcattctttactgttgccaaccagcaccg

SEQ ID NO: 653 - DNA, 40 bp, Artificial
  Synthetic primer
tggcgccctcgacaagtgctcggtgctggttggcaacagt

SEQ ID NO: 654 - DNA, 40 bp, Artificial
  Synthetic primer
agcacttgtcgagggcgccactcttgctgccactcttggc

SEQ ID NO: 655 - DNA, 40 bp, Artificial
  Synthetic primer
gaataagcgcttcccgactggccaagagtggcagcaagag

SEQ ID NO: 656 - DNA, 40 bp, Artificial
  Synthetic primer
cagtcgggaagcgcttattcatctgttgctccccaggttt

SEQ ID NO: 657 - DNA, 40 bp, Artificial
  Synthetic primer
gaatcgttggagaaagcacaaaacctggggagcaacagat

SEQ ID NO: 658 - DNA, 40 bp, Artificial
  Synthetic primer
tgtgctttctccaacgattctgggtgtcgtctggtggata

SEQ ID NO: 659 - DNA, 40 bp, Artificial
  Synthetic primer
tgttgatgttggagtcgacgtatccaccagacgacaccca

SEQ ID NO: 660 - DNA, 40 bp, Artificial
  Synthetic primer
cgtcgactccaacatcaacaccaacgagggcaggactggc

SEQ ID NO: 661 - DNA, 40 bp, Artificial
  Synthetic primer
aggacggagttgacatccttgccagtcctgccctcgttgg

SEQ ID NO: 662 - DNA, 40 bp, Artificial
  Synthetic primer
aaggatgtcaactccgtcctgacttccatccacaccttcg

SEQ ID NO: 663 - DNA, 40 bp, Artificial
  Synthetic primer
gtcacagccaaggttgggatcgaaggtgtggatggaagtc

SEQ ID NO: 664 - DNA, 40 bp, Artificial
  Synthetic primer
atcccaaccttggctgtgacgcaggcaccttccagccatg

SEQ ID NO: 665 - DNA, 40 bp, Artificial
  Synthetic primer
tggagagcgctttgtcactgcatggctggaaggtgcctgc

SEQ ID NO: 666 - DNA, 40 bp, Artificial
  Synthetic primer
cagtgacaaagcgctctccaacctcaaggttgttgtcgac

SEQ ID NO: 667 - DNA, 40 bp, Artificial
  Synthetic primer
ccgtagatggagcggaaggagtcgacaacaaccttgaggt

SEQ ID NO: 668 - DNA, 40 bp, Artificial
  Synthetic primer
tccttccgctccatctacggcgtgaacaagggcattcctg

SEQ ID NO: 669 - DNA, 40 bp, Artificial
  Synthetic primer
aatggcgacggcagcaccggcaggaatgcccttgttcacg

SEQ ID NO: 670 - DNA, 40 bp, Artificial
  Synthetic primer
ccggtgctgccgtcgccattggccggtatgcagaggatgt

SEQ ID NO: 671 - DNA, 40 bp, Artificial
  Synthetic primer
aagggttgccgttgtagtacacatcctctgcataccggcc

SEQ ID NO: 672 - DNA, 40 bp, Artificial
  Synthetic primer
gtactacaacggcaacccttggtatcttgctacatttgct

SEQ ID NO: 673 - DNA, 40 bp, Artificial
  Synthetic primer
tcgtacagctgctcggcagcagcaaatgtagcaagatacc

SEQ ID NO: 674 - DNA, 40 bp, Artificial
  Synthetic primer
gctgccgagcagctgtacgatgccatctacgtctggaaga

SEQ ID NO: 675 - DNA, 40 bp, Artificial
  Synthetic primer
caccgtgatggagcccgtcttcttccagacgtagatggca

SEQ ID NO: 676 - DNA, 40 bp, Artificial
  Synthetic primer
agacgggctccatcacggtgaccgccacctccctggcctt

SEQ ID NO: 677 - DNA, 40 bp, Artificial
  Synthetic primer
caggaacaagctcctggaagaaggccagggaggtggcggt

SEQ ID NO: 678 - DNA, 40 bp, Artificial
  Synthetic primer
cttccaggagcttgttcctggcgtgacggccgggacctac

SEQ ID NO: 679 - DNA, 40 bp, Artificial
  Synthetic primer
aaggtcgaagagctgctggagtaggtcccggccgtcacgc

SEQ ID NO: 680 - DNA, 40 bp, Artificial
  Synthetic primer
tccagcagctcttcgacctttaccaacatcatcaacgccg

SEQ ID NO: 681 - DNA, 40 bp, Artificial
  Synthetic primer
gccatcggcgtatgtcgagacggcgttgatgatgttggta

SEQ ID NO: 682 - DNA, 40 bp, Artificial
  Synthetic primer
tctcgacatacgccgatggcttcctcagcgaggctgccaa

SEQ ID NO: 683 - DNA, 40 bp, Artificial
  Synthetic primer
aaccgtcggcggggacgtacttggcagcctcgctgaggaa

SEQ ID NO: 684 - DNA, 40 bp, Artificial
  Synthetic primer
gtacgtccccgccgacggttcgctggccgagcagtttgac

SEQ ID NO: 685 - DNA, 40 bp, Artificial
  Synthetic primer
agcggagtgccgctgttgcggtcaaactgctcggccagcg

SEQ ID NO: 686 - DNA, 40 bp, Artificial
  Synthetic primer
cgcaacagcggcactccgctgtctgcgcttcacctgacgt

SEQ ID NO: 687 - DNA, 40 bp, Artificial
  Synthetic primer
caagaacgaggcgtacgaccacgtcaggtgaagcgcagac

SEQ ID NO: 688 - DNA, 40 bp, Artificial
  Synthetic primer
ggtcgtacgcctcgttcttgacagccacggcccgtcgggc

SEQ ID NO: 689 - DNA, 40 bp, Artificial
  Synthetic primer
acgaggggggcacgatgccagcccgacgggccgtggctgt

SEQ ID NO: 690 - DNA, 40 bp, Artificial
  Synthetic primer
tggcatcgtgcccccctcgtgggccaacagcagcgctagc

SEQ ID NO: 691 - DNA, 40 bp, Artificial
  Synthetic primer
gagcacgtcgaggggatcgtgctagcgctgctgttggccc

SEQ ID NO: 692 - DNA, 40 bp, Artificial
  Synthetic primer
acgatcccctcgacgtgctccggcgcgtccgtggtcggat

SEQ ID NO: 693 - DNA, 40 bp, Artificial
  Synthetic primer
ggcggtgggacgcgagtaggatccgaccacggacgcgccg

SEQ ID NO: 694 - DNA, 40 bp, Artificial
  Synthetic primer
cctactcgcgtcccaccgccacgtcattccctccgtcgca

SEQ ID NO: 695 - DNA, 40 bp, Artificial
  Synthetic primer
gcacgccaggcttgggcgtctgcgacggagggaatgacgt

SEQ ID NO: 696 - DNA, 40 bp, Artificial
  Synthetic primer
gacgcccaagcctggcgtgccttccggtactccctacacg

SEQ ID NO: 697 - DNA, 40 bp, Artificial
  Synthetic primer
ggggtcgcgcagggcaggggcgtgtagggagtaccggaag

SEQ ID NO: 698 - DNA, 40 bp, Artificial
  Synthetic primer
cccctgccctgcgcgaccccaacctccgtggccgtcacct

SEQ ID NO: 699 - DNA, 40 bp, Artificial
  Synthetic primer
tgtcgacacgagctcgtggaaggtgacggccacggaggtt

SEQ ID NO: 700 - DNA, 40 bp, Artificial
  Synthetic primer
tccacgagctcgtgtcgacacagtttggccagacggtcaa

SEQ ID NO: 701 - DNA, 40 bp, Artificial
  Synthetic primer
ccgcggcgttgcccgccaccttgaccgtctggccaaactg

SEQ ID NO: 702 - DNA, 40 bp, Artificial
  Synthetic primer
ggtggcgggcaacgccgcggccctgggcaactggagcacg

SEQ ID NO: 703 - DNA, 40 bp, Artificial
  Synthetic primer
tccagagccacggcggcgctcgtgctccagttgcccaggg

SEQ ID NO: 704 - DNA, 40 bp, Artificial
  Synthetic primer
agcgccgccgtggctctggacgccgtcaactatgccgata

SEQ ID NO: 705 - DNA, 40 bp, Artificial
  Synthetic primer
cccaatccacagggggtggttatcggcatagttgacggcg

SEQ ID NO: 706 - DNA, 40 bp, Artificial
  Synthetic primer
accaccccctgtggattgggacggtcaacctcgaggctgg

SEQ ID NO: 707 - DNA, 40 bp, Artificial
  Synthetic primer
acttgtactccacgacgtctccagcctcgaggttgaccgt

SEQ ID NO: 708 - DNA, 40 bp, Artificial
  Synthetic primer
agacgtcgtggagtacaagtacatcaatgtgggccaagat

SEQ ID NO: 709 - DNA, 40 bp, Artificial
  Synthetic primer
ctctcccaggtcacggagccatcttggcccacattgatgt

SEQ ID NO: 710 - DNA, 40 bp, Artificial
  Synthetic primer
ggctccgtgacctgggagagtgatcccaaccacacttaca

SEQ ID NO: 711 - DNA, 40 bp, Artificial
  Synthetic primer
acaagccaccgcaggaaccgtgtaagtgtggttgggatca

SEQ ID NO: 712 - DNA, 40 bp, Artificial
  Synthetic primer
cggttcctgcggtggcttgtgtgacgcaggttgtcaagga

SEQ ID NO: 713 - DNA, 40 bp, Artificial
  Synthetic primer
tttacgactgccaggtgtcctccttgacaacctgcgtcac

SEQ ID NO: 714 - DNA, 40 bp, Artificial
  Synthetic primer
ggacacctggcagtcgtaaacccagctttcttgtacaaag

SEQ ID NO: 715 - DNA, 24 bp, Artificial
  Synthetic primer
accactttgtacaagaaagctggg

SEQ ID NO: 716 - DNA, 40 bp, Artificial
  Synthetic primer
tgcggtgattgcatctcccagcacactttgcccggactac

SEQ ID NO: 717 - DNA, 40 bp, Artificial
  Synthetic primer
tctcgcgtccacatgtaatagtagtccgggcaaagtgtgc

SEQ ID NO: 718 - DNA, 40 bp, Artificial
  Synthetic primer
cgcaacagcggcactccgctgtctgcggttcacctgacgt

SEQ ID NO: 719 - DNA, 40 bp, Artificial
  Synthetic primer
caagaacgaggcgtacgaccacgtcaggtgaaccgcagac

SEQ ID NO: 720 - DNA, 40 bp, Artificial
  Synthetic primer
tgtcgacacgagcgcgtggaaggtgacggccacggaggtt

SEQ ID NO: 721 - DNA, 40 bp, Artificial
  Synthetic primer
tccacgcgctcgtgtcgacacagtttggccacacggtcaa

SEQ ID NO: 722 - DNA, 40 bp, Artificial
  Synthetic primer
ccgcggcgttgcccgccaccttgaccgtgtggccaaactg

SEQ ID NO: 723 - DNA, 40 bp, Artificial
  Synthetic primer
agcgccgccgtggctctggacgccgtcaactatagagata

SEQ ID NO: 724 - DNA, 40 bp, Artificial
  Synthetic primer
cccaatccacagggggtggttatctctatagttgacggcg

SEQ ID NO: 725 - DNA, 40 bp, Artificial
  Synthetic primer
tgcggtgattgcatctcccagcacacttgacccggactac

SEQ ID NO: 726 - DNA, 40 bp, Artificial
  Synthetic primer
tctcgcgtccacatgtaatagtagtccgggtcaagtgtgc

SEQ ID NO: 727 - DNA, 40 bp, Artificial; identical to SEQ ID NO: 718
  Synthetic primer

SEQ ID NO: 728 - DNA, 40 bp, Artificial; identical to SEQ ID NO: 719
  Synthetic primer

SEQ ID NO: 729 - DNA, 40 bp, Artificial; identical to SEQ ID NO: 720
  Synthetic primer

SEQ ID NO: 730 - DNA, 40 bp, Artificial; identical to SEQ ID NO: 721
  Synthetic primer

SEQ ID NO: 731 - DNA, 40 bp, Artificial; identical to SEQ ID NO: 722
  Synthetic primer

SEQ ID NO: 732 - DNA, 40 bp, Artificial; identical to SEQ ID NO: 723
  Synthetic primer

SEQ ID NO: 733 - DNA, 40 bp, Artificial; identical to SEQ ID NO: 724
  Synthetic primer

SEQ ID NO: 734 - DNA, 40 bp, Artificial; identical to SEQ ID NO: 716
  Synthetic primer

SEQ ID NO: 735 - DNA, 40 bp, Artificial; identical to SEQ ID NO: 717
  Synthetic primer

SEQ ID NO: 736 - DNA, 40 bp, Artificial
  Synthetic primer
ggtgaagcggtcgatgaggatcttgaagacaagagcgcta

SEQ ID NO: 737 - DNA, 40 bp, Artificial
  Synthetic primer
tcctcatcgaccgcttcaccgaaacgtacgatgcgggcct

SEQ ID NO: 738 - DNA, 40 bp, Artificial; identical to SEQ ID NO: 718
  Synthetic primer

SEQ ID NO: 739 - DNA, 40 bp, Artificial; identical to SEQ ID NO: 719
  Synthetic primer

SEQ ID NO: 740 - DNA, 40 bp, Artificial; identical to SEQ ID NO: 720
  Synthetic primer

SEQ ID NO: 741 - DNA, 40 bp, Artificial; identical to SEQ ID NO: 721
  Synthetic primer

SEQ ID NO: 742 - DNA, 40 bp, Artificial; identical to SEQ ID NO: 722
  Synthetic primer

SEQ ID NO: 743 - DNA, 40 bp, Artificial; identical to SEQ ID NO: 723
  Synthetic primer

SEQ ID NO: 744 - DNA, 40 bp, Artificial; identical to SEQ ID NO: 724
  Synthetic primer

SEQ ID NO: 745 - DNA, 40 bp, Artificial; identical to SEQ ID NO: 725
  Synthetic primer

SEQ ID NO: 746 - DNA, 40 bp, Artificial; identical to SEQ ID NO: 726
  Synthetic primer

SEQ ID NO: 747 - DNA, 40 bp, Artificial; identical to SEQ ID NO: 736
  Synthetic primer

SEQ ID NO: 748 - DNA, 40 bp, Artificial; identical to SEQ ID NO: 737
  Synthetic primer

SEQ ID NO: 749 - DNA, 40 bp, Artificial; identical to SEQ ID NO: 718
  Synthetic primer

SEQ ID NO: 750 - DNA, 40 bp, Artificial; identical to SEQ ID NO: 719
  Synthetic primer

SEQ ID NO: 751 - DNA, 40 bp, Artificial; identical to SEQ ID NO: 720
  Synthetic primer

SEQ ID NO: 752 - DNA, 40 bp, Artificial; identical to SEQ ID NO: 721
  Synthetic primer

SEQ ID NO: 753 - DNA, 40 bp, Artificial; identical to SEQ ID NO: 722
  Synthetic primer

SEQ ID NO: 754 - DNA, 40 bp, Artificial; identical to SEQ ID NO: 723
  Synthetic primer

SEQ ID NO: 755 - DNA, 40 bp, Artificial; identical to SEQ ID NO: 724
  Synthetic primer

SEQ ID NO: 756 - DNA, 40 bp, Artificial
  Synthetic primer
tgcggtgattgcatctcccagcacaagagacccggactac

SEQ ID NO: 757 - DNA, 40 bp, Artificial
  Synthetic primer
tctcgcgtccacatgtaatagtagtccgggtctcttgtgc

SEQ ID NO: 758 - DNA, 40 bp, Artificial; identical to SEQ ID NO: 718
  Synthetic primer

SEQ ID NO: 759 - DNA, 40 bp, Artificial; identical to SEQ ID NO: 719
  Synthetic primer

SEQ ID NO: 760 - DNA, 40 bp, Artificial; identical to SEQ ID NO: 720
  Synthetic primer

SEQ ID NO: 761 - DNA, 40 bp, Artificial; identical to SEQ ID NO: 721
  Synthetic primer

SEQ ID NO: 762 - DNA, 40 bp, Artificial; identical to SEQ ID NO: 722
  Synthetic primer

SEQ ID NO: 763 - DNA, 40 bp, Artificial; identical to SEQ ID NO: 723
  Synthetic primer

SEQ ID NO: 764 - DNA, 40 bp, Artificial; identical to SEQ ID NO: 724
  Synthetic primer

SEQ ID NO: 765 - DNA, 40 bp, Artificial; identical to SEQ ID NO: 756
  Synthetic primer

SEQ ID NO: 766 - DNA, 40 bp, Artificial; identical to SEQ ID NO: 757
  Synthetic primer

SEQ ID NO: 767 - DNA, 40 bp, Artificial; identical to SEQ ID NO: 736
  Synthetic primer

SEQ ID NO: 768 - DNA, 40 bp, Artificial; identical to SEQ ID NO: 737
  Synthetic primer

SEQ ID NO: 769 - DNA, 40 bp, Artificial; identical to SEQ ID NO: 718
  Synthetic primer

SEQ ID NO: 770 - DNA, 40 bp, Artificial; identical to SEQ ID NO: 719
  Synthetic primer

SEQ ID NO: 771 - DNA, 40 bp, Artificial; identical to SEQ ID NO: 720
  Synthetic primer

SEQ ID NO: 772 - DNA, 40 bp, Artificial; identical to SEQ ID NO: 721
  Synthetic primer

SEQ ID NO: 773 - DNA, 40 bp, Artificial; identical to SEQ ID NO: 722
  Synthetic primer

SEQ ID NO: 774 - DNA, 40 bp, Artificial; identical to SEQ ID NO: 723
  Synthetic primer

SEQ ID NO: 775 - DNA, 40 bp, Artificial; identical to SEQ ID NO: 724
  Synthetic primer

SEQ ID NO: 776 - DNA, 40 bp, Artificial; identical to SEQ ID NO: 756
  Synthetic primer

SEQ ID NO: 777 - DNA, 40 bp, Artificial; identical to SEQ ID NO: 757
  Synthetic primer

SEQ ID NO: 778 - DNA, 40 bp, Artificial
  Synthetic primer
cgcaacagcggcactccgctgtctgcgagacacctgacgt

SEQ ID NO: 779 - DNA, 40 bp, Artificial
  Synthetic primer
caagaacgaggcgtacgaccacgtcaggtgtctcgcagac

SEQ ID NO: 780 - DNA, 40 bp, Artificial; identical to SEQ ID NO: 720
  Synthetic primer

SEQ ID NO: 781 - DNA, 40 bp, Artificial; identical to SEQ ID NO: 721
  Synthetic primer

SEQ ID NO: 782 - DNA, 40 bp, Artificial; identical to SEQ ID NO: 722
  Synthetic primer

SEQ ID NO: 783 - DNA, 40 bp, Artificial; identical to SEQ ID NO: 723
  Synthetic primer

SEQ ID NO: 784 - DNA, 40 bp, Artificial; identical to SEQ ID NO: 724
  Synthetic primer

SEQ ID NO: 785 - DNA, 40 bp, Artificial; identical to SEQ ID NO: 756
  Synthetic primer

SEQ ID NO: 786 - DNA, 40 bp, Artificial; identical to SEQ ID NO: 757
  Synthetic primer

SEQ ID NO: 787 - DNA, 40 bp, Artificial; identical to SEQ ID NO: 736
  Synthetic primer

SEQ ID NO: 788 - DNA, 40 bp, Artificial; identical to SEQ ID NO: 737
  Synthetic primer

SEQ ID NO: 789 - DNA, 40 bp, Artificial; identical to SEQ ID NO: 778
  Synthetic primer

SEQ ID NO: 790 - DNA, 40 bp, Artificial; identical to SEQ ID NO: 779
  Synthetic primer

SEQ ID NO: 791 - DNA, 40 bp, Artificial; identical to SEQ ID NO: 720
  Synthetic primer

SEQ ID NO: 792 - DNA, 40 bp, Artificial; identical to SEQ ID NO: 721
  Synthetic primer

SEQ ID NO: 793 - DNA, 40 bp, Artificial; identical to SEQ ID NO: 722
  Synthetic primer

SEQ ID NO: 794 - DNA, 40 bp, Artificial; identical to SEQ ID NO: 723
  Synthetic primer

SEQ ID NO: 795 - DNA, 40 bp, Artificial; identical to SEQ ID NO: 724
  Synthetic primer

SEQ ID NO: 796 - DNA, 40 bp, Artificial; identical to SEQ ID NO: 718
  Synthetic primer

SEQ ID NO: 797 - DNA, 40 bp, Artificial; identical to SEQ ID NO: 719
  Synthetic primer

SEQ ID NO: 798 - DNA, 40 bp, Artificial
  Synthetic primer
ggtcgtacgcctcgttcttgacagccgcgctccgtcgggc

SEQ ID NO: 799 - DNA, 40 bp, Artificial
  Synthetic primer
acgaggggggcacgatgccagcccgacggagcgcggctgt

SEQ ID NO: 800 - DNA, 40 bp, Artificial
  Synthetic primer
tccacgagctcgtgtcgacacagtttggccacacggtcaa

SEQ ID NO: 801 - DNA, 40 bp, Artificial; identical to SEQ ID NO: 722
  Synthetic primer

SEQ ID NO: 802 - DNA, 40 bp, Artificial
  Synthetic primer
agcgccgccgtggctctggaccgcgtcaactatcgcgata

SEQ ID NO: 803 - DNA, 40 bp, Artificial
  Synthetic primer
cccaatccacagggggtggttatcgcgatagttgacgcgg

SEQ ID NO: 804 - DNA, 40 bp, Artificial
  Synthetic primer
agacgtcgtggagtacaagtacatcattgtgggccaagat

SEQ ID NO: 805 - DNA, 40 bp, Artificial
  Synthetic primer
ctctcccaggtcacggagccatcttggcccacaatgatgt

SEQ ID NO: 806 - DNA, 40 bp, Artificial; identical to SEQ ID NO: 718
  Synthetic primer

SEQ ID NO: 807 - DNA, 40 bp, Artificial; identical to SEQ ID NO: 719
  Synthetic primer

SEQ ID NO: 808 - DNA, 40 bp, Artificial
  Synthetic primer
ggtcgtacgcctcgttcttgacagccgcgcaacgtcgggc

SEQ ID NO: 809 - DNA, 40 bp, Artificial
  Synthetic primer
acgaggggggcacgatgccagcccgacgttgcgcggctgt

SEQ ID NO: 810 - DNA, 40 bp, Artificial; identical to SEQ ID NO: 800
  Synthetic primer

SEQ ID NO: 811 - DNA, 40 bp, Artificial; identical to SEQ ID NO: 722
  Synthetic primer

SEQ ID NO: 812 - DNA, 40 bp, Artificial; identical to SEQ ID NO: 802
  Synthetic primer

SEQ ID NO: 813 - DNA, 40 bp, Artificial; identical to SEQ ID NO: 803
  Synthetic primer

SEQ ID NO: 814 - DNA, 40 bp, Artificial; identical to SEQ ID NO: 804
  Synthetic primer

SEQ ID NO: 815 - DNA, 40 bp, Artificial; identical to SEQ ID NO: 805
  Synthetic primer

SEQ ID NO: 816 - DNA, 40 bp, Artificial; identical to SEQ ID NO: 718
  Synthetic primer

SEQ ID NO: 817 - DNA, 40 bp, Artificial; identical to SEQ ID NO: 719
  Synthetic primer

SEQ ID NO: 818 - DNA, 40 bp, Artificial
  Synthetic primer
ggtcgtacgcctcgttcttgacagccgcggcccgtcgggc

SEQ ID NO: 819 - DNA, 40 bp, Artificial
  Synthetic primer
acgaggggggcacgatgccagcccgacgggccgcggctgt

SEQ ID NO: 820 - DNA, 40 bp, Artificial; identical to SEQ ID NO: 800
  Synthetic primer

SEQ ID NO: 821 - DNA, 40 bp, Artificial; identical to SEQ ID NO: 722
  Synthetic primer

SEQ ID NO: 822 - DNA, 40 bp, Artificial
  Synthetic primer
agcgccgccgtggctctggaccgcgtcaactatgccgata

SEQ ID NO: 823 - DNA, 40 bp, Artificial
  Synthetic primer
cccaatccacagggggtggttatcggcatagttgacgcgg

SEQ ID NO: 824 - DNA, 40 bp, Artificial; identical to SEQ ID NO: 804
  Synthetic primer

SEQ ID NO: 825 - DNA, 40 bp, Artificial; identical to SEQ ID NO: 805
  Synthetic primer

SEQ ID NO: 826 - DNA, 40 bp, Artificial; identical to SEQ ID NO: 718
  Synthetic primer

SEQ ID NO: 827 - DNA, 40 bp, Artificial; identical to SEQ ID NO: 719
  Synthetic primer

SEQ ID NO: 828 - DNA, 40 bp, Artificial; identical to SEQ ID NO: 818
  Synthetic primer

SEQ ID NO: 829 - DNA, 40 bp, Artificial; identical to SEQ ID NO: 819
  Synthetic primer

SEQ ID NO: 830 - DNA, 40 bp, Artificial; identical to SEQ ID NO: 800
  Synthetic primer

SEQ ID NO: 831 - DNA, 40 bp, Artificial; identical to SEQ ID NO: 722
  Synthetic primer

SEQ ID NO: 832 - DNA, 40 bp, Artificial
  Synthetic primer
agcgccgccgtggctctggacgccgtcaactatcgcgata

SEQ ID NO: 833 - DNA, 40 bp, Artificial
  Synthetic primer
cccaatccacagggggtggttatcgcgatagttgacggcg

SEQ ID NO: 834 - DNA, 40 bp, Artificial; identical to SEQ ID NO: 804
  Synthetic primer

SEQ ID NO: 835 - DNA, 40 bp, Artificial; identical to SEQ ID NO: 805
  Synthetic primer

SEQ ID NO: 836 - DNA, 40 bp, Artificial
  Synthetic primer
cagtagatggagcggaaggagtcgacaacaaccttgaggt

SEQ ID NO: 837 - DNA, 40 bp, Artificial
  Synthetic primer
tccttccgctccatctactgcgtgaacaagggcattcctg

SEQ ID NO: 838 - DNA, 40 bp, Artificial; identical to SEQ ID NO: 778
  Synthetic primer

SEQ ID NO: 839 - DNA, 40 bp, Artificial; identical to SEQ ID NO: 779
  Synthetic primer

SEQ ID NO: 840 - DNA, 40 bp, Artificial
  Synthetic primer
ggtcgtacgcctcgttcttgacagccacgctccgtcgggc

SEQ ID NO: 841 - DNA, 40 bp, Artificial
  Synthetic primer
acgaggggggcacgatgccagcccgacggagcgtggctgt

SEQ ID NO: 842 - DNA, 40 bp, Artificial; identical to SEQ ID NO: 836
  Synthetic primer

SEQ ID NO: 843 - DNA, 40 bp, Artificial; identical to SEQ ID NO: 837
  Synthetic primer

SEQ ID NO: 844 - DNA, 40 bp, Artificial
  Synthetic primer
cgcaacagcggcactccgctgtctgcggtacacctgacgt

SEQ ID NO: 845 - DNA, 40 bp, Artificial
  Synthetic primer
caagaacgaggcgtacgaccacgtcaggtgtaccgcagac

SEQ ID NO: 846 - DNA, 40 bp, Artificial
  Synthetic primer
ggtcgtacgcctcgttcttgacagccacgcagcgtcgggc

SEQ ID NO: 847 - DNA, 40 bp, Artificial
  Synthetic primer
acgaggggggcacgatgccagcccgacgctgcgtggctgt

SEQ ID NO: 848 - DNA, 40 bp, Artificial; identical to SEQ ID NO: 836
  Synthetic primer

SEQ ID NO: 849 - DNA, 40 bp, Artificial; identical to SEQ ID NO: 837
  Synthetic primer

SEQ ID NO: 850 - DNA, 40 bp, Artificial; identical to SEQ ID NO: 844
  Synthetic primer

SEQ ID NO: 851 - DNA, 40 bp, Artificial; identical to SEQ ID NO: 845
  Synthetic primer

SEQ ID NO: 852 - DNA, 40 bp, Artificial
  Synthetic primer
ggtcgtacgcctcgttcttgacagccacgttacgtcgggc

SEQ ID NO: 853 - DNA, 40 bp, Artificial
  Synthetic primer
acgaggggggcacgatgccagcccgacgtaacgtggctgt

SEQ ID NO: 854 - DNA, 40 bp, Artificial; identical to SEQ ID NO: 800
  Synthetic primer

SEQ ID NO: 855 - DNA, 40 bp, Artificial; identical to SEQ ID NO: 722
  Synthetic primer

SEQ ID NO: 856 - DNA, 40 bp, Artificial; identical to SEQ ID NO: 836
  Synthetic primer

SEQ ID NO: 857 - DNA, 40 bp, Artificial; identical to SEQ ID NO: 837
  Synthetic primer

SEQ ID NO: 858 - DNA, 40 bp, Artificial; identical to SEQ ID NO: 778
  Synthetic primer

SEQ ID NO: 859 - DNA, 40 bp, Artificial; identical to SEQ ID NO: 779
  Synthetic primer

SEQ ID NO: 860 - DNA, 40 bp, Artificial; identical to SEQ ID NO: 846
  Synthetic primer

SEQ ID NO: 861 - DNA, 40 bp, Artificial; identical to SEQ ID NO: 847
  Synthetic primer

SEQ ID NO: 862 - DNA, 40 bp, Artificial; identical to SEQ ID NO: 800
  Synthetic primer

SEQ ID NO: 863 - DNA, 40 bp, Artificial; identical to SEQ ID NO: 722
  Synthetic primer

SEQ ID NO: 864 - DNA, 40 bp, Artificial
  Synthetic primer
cgcaacagcggcactccgctgtctgcgcgtcacctgacgt

SEQ ID NO: 865 - DNA, 40 bp, Artificial
  Synthetic primer
caagaacgaggcgtacgaccacgtcaggtgacgcgcagac

SEQ ID NO: 866 - DNA, 40 bp, Artificial; identical to SEQ ID NO: 840
  Synthetic primer

SEQ ID NO: 867 - DNA, 40 bp, Artificial; identical to SEQ ID NO: 841
  Synthetic primer

SEQ ID NO: 868 - DNA, 40 bp, Artificial; identical to SEQ ID NO: 800
  Synthetic primer

SEQ ID NO: 869 - DNA, 40 bp, Artificial; identical to SEQ ID NO: 722
  Synthetic primer

SEQ ID NO: 870 - DNA, 40 bp, Artificial; identical to SEQ ID NO: 718
  Synthetic primer

SEQ ID NO: 871 - DNA, 40 bp, Artificial; identical to SEQ ID NO: 719
  Synthetic primer

SEQ ID NO: 872 - DNA, 40 bp, Artificial
  Synthetic primer
ggtcgtacgcctcgttcttgacagccacgcttcgtcgggc

SEQ ID NO: 873 - DNA, 40 bp, Artificial
  Synthetic primer
acgaggggggcacgatgccagcccgacgaagcgtggctgt

SEQ ID NO: 874 - DNA, 40 bp, Artificial
  Synthetic primer
tccacgagctcgtgtcgacacagtttggccatacggtcaa

SEQ ID NO: 875 - DNA, 40 bp, Artificial
  Synthetic primer
ccgcggcgttgcccgccaccttgaccgtatggccaaactg

SEQ ID NO: 876 - DNA, 40 bp, Artificial; identical to SEQ ID NO: 756
  Synthetic primer

SEQ ID NO: 877 - DNA, 40 bp, Artificial; identical to SEQ ID NO: 757
  Synthetic primer

SEQ ID NO: 878 - DNA, 40 bp, Artificial
  Synthetic primer
ggtcgtacgcctcgttcttgacagccgcagcccgtcgggc

SEQ ID NO: 879 - DNA, 40 bp, Artificial
  Synthetic primer
acgaggggggcacgatgccagcccgacgggctgcggctgt

SEQ ID NO: 880 - DNA, 40 bp, Artificial
  Synthetic primer
tgtcgacacgagcacgtggaaggtgacggccacggaggtt

SEQ ID NO: 881 - DNA, 40 bp, Artificial
  Synthetic primer
tccacgtgctcgtgtcgacacagtttggccagacggtcaa

SEQ ID NO: 882 - DNA, 40 bp, Artificial; identical to SEQ ID NO: 822
  Synthetic primer

SEQ ID NO: 883 - DNA, 40 bp, Artificial; identical to SEQ ID NO: 823
  Synthetic primer

SEQ ID NO: 884 - DNA, 40 bp, Artificial
  Synthetic primer
agacgtcgtggagtacaagtacatcaaagtgggccaagat

SEQ ID NO: 885 - DNA, 40 bp, Artificial
  Synthetic primer
ctctcccaggtcacggagccatcttggcccactttgatgt

SEQ ID NO: 886 - DNA, 40 bp, Artificial
  Synthetic primer
tgcggtgattgcatctcccagcacaattagaccggactac

SEQ ID NO: 887 - DNA, 40 bp, Artificial
  Synthetic primer
tctcgcgtccacatgtaatagtagtccggtctaattgtgc

SEQ ID NO: 888 - DNA, 40 bp, Artificial; identical to SEQ ID NO: 720
  Synthetic primer

SEQ ID NO: 889 - DNA, 40 bp, Artificial; identical to SEQ ID NO: 721
  Synthetic primer

SEQ ID NO: 890 - DNA, 40 bp, Artificial; identical to SEQ ID NO: 722
  Synthetic primer

SEQ ID NO: 891 - DNA, 40 bp, Artificial; identical to SEQ ID NO: 804
  Synthetic primer

SEQ ID NO: 892 - DNA, 40 bp, Artificial; identical to SEQ ID NO: 805
  Synthetic primer

SEQ ID NO: 893 - DNA, 40 bp, Artificial; identical to SEQ ID NO: 720
  Synthetic primer

SEQ ID NO: 894 - DNA, 40 bp, Artificial
  Synthetic primer
tccacgcgctcgtgtcgacacagtttggccagacggtcaa

SEQ ID NO: 895 - DNA, 40 bp, Artificial; identical to SEQ ID NO: 804
  Synthetic primer

SEQ ID NO: 896 - DNA, 40 bp, Artificial; identical to SEQ ID NO: 805
  Synthetic primer

SEQ ID NO: 897 - DNA, 40 bp, Artificial; identical to SEQ ID NO: 756
  Synthetic primer

SEQ ID NO: 898 - DNA, 40 bp, Artificial; identical to SEQ ID NO: 757
  Synthetic primer

SEQ ID NO: 899 - DNA, 40 bp, Artificial; identical to SEQ ID NO: 818
  Synthetic primer

SEQ ID NO: 900 - DNA, 40 bp, Artificial; identical to SEQ ID NO: 819
  Synthetic primer

SEQ ID NO: 901 - DNA, 40 bp, Artificial; identical to SEQ ID NO: 720
  Synthetic primer

SEQ ID NO: 902 - DNA, 40 bp, Artificial; identical to SEQ ID NO: 721
  Synthetic primer

SEQ ID NO: 903 - DNA, 40 bp, Artificial; identical to SEQ ID NO: 722
  Synthetic primer

SEQ ID NO: 904 - DNA, 40 bp, Artificial; identical to SEQ ID NO: 804
  Synthetic primer

SEQ ID NO: 905 - DNA, 40 bp, Artificial; identical to SEQ ID NO: 805
  Synthetic primer

SEQ ID NO: 906 - DNA, 40 bp, Artificial
  Synthetic primer
tgcggtgattgcatctcccagcacaattcgcccggactac

SEQ ID NO: 907 - DNA, 40 bp, Artificial
  Synthetic primer
tctcgcgtccacatgtaatagtagtccgggcgaattgtgc

SEQ ID NO: 908 - DNA, 40 bp, Artificial; identical to SEQ ID NO: 818
  Synthetic primer

SEQ ID NO: 909 - DNA, 40 bp, Artificial; identical to SEQ ID NO: 819
  Synthetic primer

SEQ ID NO: 910 - DNA, 40 bp, Artificial; identical to SEQ ID NO: 800
  Synthetic primer

SEQ ID NO: 911 - DNA, 40 bp, Artificial; identical to SEQ ID NO: 722
  Synthetic primer

SEQ ID NO: 912 - DNA, 40 bp, Artificial
  Synthetic primer
agcgccgccgtggctctggacgcggtcaactatgccgata

SEQ ID NO: 913 - DNA, 40 bp, Artificial
  Synthetic primer
cccaatccacagggggtggttatcggcatagttgaccgcg

SEQ ID NO: 914 - DNA, 33 bp, Artificial
  Synthetic primer
gagaggggacaagtttgtacaaaaaagcaggct

SEQ ID NO: 915 - DNA, 40 bp, Artificial; identical to SEQ ID NO: 619
  Synthetic primer

SEQ ID NO: 916 - DNA, 40 bp, Artificial; identical to SEQ ID NO: 620
  Synthetic primer

SEQ ID NO: 917 - DNA, 40 bp, Artificial; identical to SEQ ID NO: 621
  Synthetic primer

SEQ ID NO: 918 - DNA, 40 bp, Artificial; identical to SEQ ID NO: 622
  Synthetic primer

SEQ ID NO: 919 - DNA, 40 bp, Artificial; identical to SEQ ID NO: 623
  Synthetic primer

SEQ ID NO: 920 - DNA, 40 bp, Artificial; identical to SEQ ID NO: 624
  Synthetic primer

SEQ ID NO: 921 - DNA, 40 bp, Artificial; identical to SEQ ID NO: 625
  Synthetic primer

SEQ ID NO: 922 - DNA, 40 bp, Artificial; identical to SEQ ID NO: 626
  Synthetic primer

SEQ ID NO: 923 - DNA, 40 bp, Artificial; identical to SEQ ID NO: 627
  Synthetic primer

SEQ ID NO: 924 - DNA, 40 bp, Artificial; identical to SEQ ID NO: 628
  Synthetic primer

SEQ ID NO: 925 - DNA, 40 bp, Artificial; identical to SEQ ID NO: 629
  Synthetic primer

SEQ ID NO: 926 - DNA, 40 bp, Artificial
  Synthetic primer
tgcggtgattgcatctcccagcacacaagacccggactac

SEQ ID NO: 927 - DNA, 40 bp, Artificial
  Synthetic primer
tctcgcgtccacatgtaatagtagtccgggtcttgtgtgc

SEQ ID NO: 928 - DNA, 40 bp, Artificial; identical to SEQ ID NO: 632
  Synthetic primer

SEQ ID NO: 929 - DNA, 40 bp, Artificial; identical to SEQ ID NO: 633
  Synthetic primer

SEQ ID NO: 930 - DNA, 40 bp, Artificial; identical to SEQ ID NO: 634
  Synthetic primer

SEQ ID NO: 931 - DNA, 40 bp, Artificial; identical to SEQ ID NO: 635
  Synthetic primer

SEQ ID NO: 932 - DNA, 40 bp, Artificial; identical to SEQ ID NO: 636
  Synthetic primer

SEQ ID NO: 933 - DNA, 40 bp, Artificial; identical to SEQ ID NO: 637
  Synthetic primer

SEQ ID NO: 934 - DNA, 40 bp, Artificial; identical to SEQ ID NO: 638
  Synthetic primer

SEQ ID NO: 935 - DNA, 40 bp, Artificial; identical to SEQ ID NO: 639
  Synthetic primer

SEQ ID NO: 936 - DNA, 40 bp, Artificial; identical to SEQ ID NO: 640
  Synthetic primer

SEQ ID NO: 937 - DNA, 40 bp, Artificial; identical to SEQ ID NO: 641
  Synthetic primer

SEQ ID NO: 938 - DNA, 40 bp, Artificial; identical to SEQ ID NO: 642
  Synthetic primer

SEQ ID NO: 939 - DNA, 40 bp, Artificial; identical to SEQ ID NO: 643
  Synthetic primer

SEQ ID NO: 940 - DNA, 40 bp, Artificial; identical to SEQ ID NO: 644
  Synthetic primer

SEQ ID NO: 941 - DNA, 40 bp, Artificial; identical to SEQ ID NO: 645
  Synthetic primer

SEQ ID NO: 942 - DNA, 40 bp, Artificial; identical to SEQ ID NO: 646
  Synthetic primer

SEQ ID NO: 943 - DNA, 40 bp, Artificial; identical to SEQ ID NO: 647
  Synthetic primer

SEQ ID NO: 944 - DNA, 40 bp, Artificial; identical to SEQ ID NO: 648
  Synthetic primer

SEQ ID NO: 945 - DNA, 40 bp, Artificial; identical to SEQ ID NO: 649
  Synthetic primer

SEQ ID NO: 946 - DNA, 40 bp, Artificial; identical to SEQ ID NO: 650
  Synthetic primer

SEQ ID NO: 947 - DNA, 40 bp, Artificial; identical to SEQ ID NO: 651
  Synthetic primer

SEQ ID NO: 948 - DNA, 40 bp, Artificial; identical to SEQ ID NO: 652
  Synthetic primer

SEQ ID NO: 949 - DNA, 40 bp, Artificial; identical to SEQ ID NO: 653
  Synthetic primer

SEQ ID NO: 950 - DNA, 40 bp, Artificial; identical to SEQ ID NO: 654
  Synthetic primer

SEQ ID NO: 951 - DNA, 40 bp, Artificial; identical to SEQ ID NO: 655
  Synthetic primer

SEQ ID NO: 952 - DNA, 40 bp, Artificial; identical to SEQ ID NO: 656
  Synthetic primer

SEQ ID NO: 953 - DNA, 40 bp, Artificial; identical to SEQ ID NO: 657
  Synthetic primer

SEQ ID NO: 954 - DNA, 40 bp, Artificial; identical to SEQ ID NO: 658
  Synthetic primer

SEQ ID NO: 955 - DNA, 40 bp, Artificial; identical to SEQ ID NO: 659
  Synthetic primer

SEQ ID NO: 956 - DNA, 40 bp, Artificial; identical to SEQ ID NO: 660
  Synthetic primer

SEQ ID NO: 957 - DNA, 40 bp, Artificial; identical to SEQ ID NO: 661
  Synthetic primer

SEQ ID NO: 958 - DNA, 40 bp, Artificial; identical to SEQ ID NO: 662
  Synthetic primer

SEQ ID NO: 959 - DNA, 40 bp, Artificial; identical to SEQ ID NO: 663
  Synthetic primer

SEQ ID NO: 960 - DNA, 40 bp, Artificial; identical to SEQ ID NO: 664
  Synthetic primer

SEQ ID NO: 961 - DNA, 40 bp, Artificial; identical to SEQ ID NO: 665
  Synthetic primer

SEQ ID NO: 962 - DNA, 40 bp, Artificial; identical to SEQ ID NO: 666
  Synthetic primer

SEQ ID NO: 963 - DNA, 40 bp, Artificial; identical to SEQ ID NO: 667
  Synthetic primer

SEQ ID NO: 964 - DNA, 40 bp, Artificial; identical to SEQ ID NO: 668
  Synthetic primer

SEQ ID NO: 965 - DNA, 40 bp, Artificial; identical to SEQ ID NO: 669
  Synthetic primer

SEQ ID NO: 966 - DNA, 40 bp, Artificial; identical to SEQ ID NO: 670
  Synthetic primer

SEQ ID NO: 967 - DNA, 40 bp, Artificial; identical to SEQ ID NO: 671
  Synthetic primer

SEQ ID NO: 968 - DNA, 40 bp, Artificial; identical to SEQ ID NO: 672
  Synthetic primer

SEQ ID NO: 969 - DNA, 40 bp, Artificial; identical to SEQ ID NO: 673
  Synthetic primer

SEQ ID NO: 970 - DNA, 40 bp, Artificial; identical to SEQ ID NO: 674
  Synthetic primer

SEQ ID NO: 971 - DNA, 40 bp, Artificial; identical to SEQ ID NO: 675
  Synthetic primer

SEQ ID NO: 972 - DNA, 40 bp, Artificial; identical to SEQ ID NO: 676
  Synthetic primer

SEQ ID NO: 973 - DNA, 40 bp, Artificial; identical to SEQ ID NO: 677
  Synthetic primer

SEQ ID NO: 974 - DNA, 40 bp, Artificial; identical to SEQ ID NO: 678
  Synthetic primer

SEQ ID NO: 975 - DNA, 40 bp, Artificial; identical to SEQ ID NO: 679
  Synthetic primer

SEQ ID NO: 976 - DNA, 40 bp, Artificial; identical to SEQ ID NO: 680
  Synthetic primer

SEQ ID NO: 977 - DNA, 40 bp, Artificial; identical to SEQ ID NO: 681
  Synthetic primer

SEQ ID NO: 978 - DNA, 40 bp, Artificial; identical to SEQ ID NO: 682
  Synthetic primer

SEQ ID NO: 979 - DNA, 40 bp, Artificial; identical to SEQ ID NO: 683
  Synthetic primer

SEQ ID NO: 980 - DNA, 40 bp, Artificial; identical to SEQ ID NO: 684
  Synthetic primer

SEQ ID NO: 981 - DNA, 40 bp, Artificial; identical to SEQ ID NO: 685
  Synthetic primer

SEQ ID NO: 982 - DNA, 40 bp, Artificial; identical to SEQ ID NO: 686
  Synthetic primer

SEQ ID NO: 983 - DNA, 40 bp, Artificial; identical to SEQ ID NO: 687
  Synthetic primer

SEQ ID NO: 984 - DNA, 40 bp, Artificial; identical to SEQ ID NO: 688
  Synthetic primer

SEQ ID NO: 985 - DNA, 40 bp, Artificial; identical to SEQ ID NO: 689
  Synthetic primer

SEQ ID NO: 986 - DNA, 40 bp, Artificial; identical to SEQ ID NO: 690
  Synthetic primer

SEQ ID NO: 987 - DNA, 40 bp, Artificial; identical to SEQ ID NO: 691
  Synthetic primer

SEQ ID NO: 988 - DNA, 40 bp, Artificial; identical to SEQ ID NO: 692
  Synthetic primer

SEQ ID NO: 989 - DNA, 40 bp, Artificial; identical to SEQ ID NO: 693
  Synthetic primer

SEQ ID NO: 990 - DNA, 40 bp, Artificial; identical to SEQ ID NO: 694
  Synthetic primer

SEQ ID NO: 991 - DNA, 40 bp, Artificial; identical to SEQ ID NO: 695
  Synthetic primer

SEQ ID NO: 992 - DNA, 40 bp, Artificial; identical to SEQ ID NO: 696
  Synthetic primer

SEQ ID NO: 993 - DNA, 40 bp, Artificial; identical to SEQ ID NO: 697
  Synthetic primer

SEQ ID NO: 994 - DNA, 40 bp, Artificial; identical to SEQ ID NO: 698
  Synthetic primer

SEQ ID NO: 995 - DNA, 40 bp, Artificial; identical to SEQ ID NO: 699
  Synthetic primer

SEQ ID NO: 996 - DNA, 40 bp, Artificial; identical to SEQ ID NO: 800
  Synthetic primer

SEQ ID NO: 997 - DNA, 40 bp, Artificial; identical to SEQ ID NO: 722
  Synthetic primer

SEQ ID NO: 998 - DNA, 40 bp, Artificial; identical to SEQ ID NO: 702
  Synthetic primer

SEQ ID NO: 999 - DNA, 40 bp, Artificial; identical to SEQ ID NO: 703
  Synthetic primer

SEQ ID NO: 1000 - DNA, 40 bp, Artificial; identical to SEQ ID NO: 704
  Synthetic primer

SEQ ID NO: 1001 - DNA, 40 bp, Artificial; identical to SEQ ID NO: 705
  Synthetic primer

SEQ ID NO: 1002 - DNA, 40 bp, Artificial; identical to SEQ ID NO: 706
  Synthetic primer

SEQ ID NO: 1003 - DNA, 40 bp, Artificial; identical to SEQ ID NO: 707
  Synthetic primer

SEQ ID NO: 1004 - DNA, 40 bp, Artificial; identical to SEQ ID NO: 708
  Synthetic primer

SEQ ID NO: 1005 - DNA, 40 bp, Artificial; identical to SEQ ID NO: 709
  Synthetic primer

SEQ ID NO: 1006 - DNA, 40 bp, Artificial; identical to SEQ ID NO: 710
  Synthetic primer

SEQ ID NO: 1007 - DNA, 40 bp, Artificial; identical to SEQ ID NO: 711
  Synthetic primer

SEQ ID NO: 1008 - DNA, 40 bp, Artificial; identical to SEQ ID NO: 712
  Synthetic primer

SEQ ID NO: 1009 - DNA, 40 bp, Artificial; identical to SEQ ID NO: 713
  Synthetic primer

SEQ ID NO: 1010 - DNA, 40 bp, Artificial; identical to SEQ ID NO: 714
  Synthetic primer

SEQ ID NO: 1011 - DNA, 32 bp, Artificial
  Synthetic primer
ctctggggaccactttgtacaagaaagctggg

SEQ ID NO: 1012 - DNA, 40 bp, Artificial; identical to SEQ ID NO: 736
  Synthetic primer

SEQ ID NO: 1013 - DNA, 40 bp, Artificial; identical to SEQ ID NO: 737
  Synthetic primer

SEQ ID NO: 1014 - DNA, 40 bp, Artificial; identical to SEQ ID NO: 818
  Synthetic primer

SEQ ID NO: 1015 - DNA, 40 bp, Artificial; identical to SEQ ID NO: 819
  Synthetic primer

SEQ ID NO: 1016 - DNA, 40 bp, Artificial; identical to SEQ ID NO: 718
  Synthetic primer

SEQ ID NO: 1017 - DNA, 40 bp, Artificial; identical to SEQ ID NO: 719
  Synthetic primer

SEQ ID NO: 1018 - DNA, 40 bp, Artificial; identical to SEQ ID NO: 818
  Synthetic primer

SEQ ID NO: 1019 - DNA, 40 bp, Artificial; identical to SEQ ID NO: 819
  Synthetic primer

SEQ ID NO: 1020 - DNA, 40 bp, Artificial; identical to SEQ ID NO: 798
  Synthetic primer

SEQ ID NO: 1021 - DNA, 40 bp, Artificial; identical to SEQ ID NO: 799
  Synthetic primer

SEQ ID NO: 1022 - DNA, 40 bp, Artificial; identical to SEQ ID NO: 818
  Synthetic primer

SEQ ID NO: 1023 - DNA, 40 bp, Artificial; identical to SEQ ID NO: 819
  Synthetic primer

SEQ ID NO: 1024 - DNA, 40 bp, Artificial; identical to SEQ ID NO: 720
  Synthetic primer

SEQ ID NO: 1025 - DNA, 40 bp, Artificial; identical to SEQ ID NO: 721
  Synthetic primer

SEQ ID NO: 1026 - DNA, 40 bp, Artificial; identical to SEQ ID NO: 818
  Synthetic primer

SEQ ID NO: 1027 - DNA, 40 bp, Artificial; identical to SEQ ID NO: 819
  Synthetic primer

SEQ ID NO: 1028 - DNA, 40 bp, Artificial; identical to SEQ ID NO: 818
  Synthetic primer

SEQ ID NO: 1029 - DNA, 40 bp, Artificial; identical to SEQ ID NO: 819
  Synthetic primer

SEQ ID NO: 1030 - DNA, 40 bp, Artificial; identical to SEQ ID NO: 832
  Synthetic primer

SEQ ID NO: 1031 - DNA, 40 bp, Artificial; identical to SEQ ID NO: 833
  Synthetic primer

SEQ ID NO: 1032 - DNA, 40 bp, Artificial; identical to SEQ ID NO: 736
  Synthetic primer

SEQ ID NO: 1033 - DNA, 40 bp, Artificial; identical to SEQ ID NO: 737
  Synthetic primer

SEQ ID NO: 1034 - DNA, 40 bp, Artificial; identical to SEQ ID NO: 818
  Synthetic primer

SEQ ID NO: 1035 - DNA, 40 bp, Artificial; identical to SEQ ID NO: 819
  Synthetic primer

SEQ ID NO: 1036 - DNA, 40 bp, Artificial; identical to SEQ ID NO: 832
  Synthetic primer

SEQ ID NO: 1037 - DNA, 40 bp, Artificial; identical to SEQ ID NO: 833
  Synthetic primer

SEQ ID NO: 1038 - DNA, 40 bp, Artificial; identical to SEQ ID NO: 718
  Synthetic primer

SEQ ID NO: 1039 - DNA, 40 bp, Artificial; identical to SEQ ID NO: 719
  Synthetic primer

SEQ ID NO: 1040 - DNA, 40 bp, Artificial; identical to SEQ ID NO: 818
  Synthetic primer

SEQ ID NO: 1041 - DNA, 40 bp, Artificial; identical to SEQ ID NO: 819
  Synthetic primer

SEQ ID NO: 1042 - DNA, 40 bp, Artificial; identical to SEQ ID NO: 832
  Synthetic primer

SEQ ID NO: 1043 - DNA, 40 bp, Artificial; identical to SEQ ID NO: 833
  Synthetic primer

SEQ ID NO: 1044 - DNA, 40 bp, Artificial; identical to SEQ ID NO: 798
  Synthetic primer

SEQ ID NO: 1045 - DNA, 40 bp, Artificial; identical to SEQ ID NO: 799
  Synthetic primer

SEQ ID NO: 1046 - DNA, 40 bp, Artificial; identical to SEQ ID NO: 832
  Synthetic primer

SEQ ID NO: 1047 - DNA, 40 bp, Artificial; identical to SEQ ID NO: 833
  Synthetic primer

SEQ ID NO: 1048 - DNA, 40 bp, Artificial; identical to SEQ ID NO: 798
  Synthetic primer

SEQ ID NO: 1049 - DNA, 40 bp, Artificial; identical to SEQ ID NO: 799
  Synthetic primer

SEQ ID NO: 1050 - DNA, 40 bp, Artificial; identical to SEQ ID NO: 720
  Synthetic primer

SEQ ID NO: 1051 - DNA, 40 bp, Artificial; identical to SEQ ID NO: 721
  Synthetic primer

SEQ ID NO: 1052 - DNA, 40 bp, Artificial; identical to SEQ ID NO: 736
  Synthetic primer

SEQ ID NO: 1053 - DNA, 40 bp, Artificial; identical to SEQ ID NO: 737
  Synthetic primer

SEQ ID NO: 1054 - DNA, 40 bp, Artificial; identical to SEQ ID NO: 798
  Synthetic primer

SEQ ID NO: 1055 - DNA, 40 bp, Artificial; identical to SEQ ID NO: 799
  Synthetic primer

SEQ ID NO: 1056 - DNA, 40 bp, Artificial; identical to SEQ ID NO: 832
  Synthetic primer

SEQ ID NO: 1057 - DNA, 40 bp, Artificial; identical to SEQ ID NO: 833
  Synthetic primer

SEQ ID NO: 1058 - DNA, 40 bp, Artificial; identical to SEQ ID NO: 718
  Synthetic primer

SEQ ID NO: 1059 - DNA, 40 bp, Artificial; identical to SEQ ID NO: 719
  Synthetic primer

SEQ ID NO: 1060 - DNA, 40 bp, Artificial; identical to SEQ ID NO: 798
  Synthetic primer

SEQ ID NO: 1061 - DNA, 40 bp, Artificial; identical to SEQ ID NO: 799
  Synthetic primer

SEQ ID NO: 1062 - DNA, 40 bp, Artificial; identical to SEQ ID NO: 832
  Synthetic primer

SEQ ID NO: 1063 - DNA, 40 bp, Artificial; identical to SEQ ID NO: 833
  Synthetic primer

SEQ ID NO: 1064 - DNA, 40 bp, Artificial; identical to SEQ ID NO: 736
  Synthetic primer

SEQ ID NO: 1065 - DNA, 40 bp, Artificial; identical to SEQ ID NO: 737
  Synthetic primer

SEQ ID NO: 1066 - DNA, 40 bp, Artificial; identical to SEQ ID NO: 718
  Synthetic primer

SEQ ID NO: 1067 - DNA, 40 bp, Artificial; identical to SEQ ID NO: 719
  Synthetic primer

SEQ ID NO: 1068 - DNA, 40 bp, Artificial; identical to SEQ ID NO: 840
  Synthetic primer

SEQ ID NO: 1069 - DNA, 40 bp, Artificial; identical to SEQ ID NO: 841
  Synthetic primer

SEQ ID NO: 1070 - DNA, 40 bp, Artificial; identical to SEQ ID NO: 832
  Synthetic primer

SEQ ID NO: 1071 - DNA, 40 bp, Artificial; identical to SEQ ID NO: 833
  Synthetic primer

SEQ ID NO: 1072 - DNA, 40 bp, Artificial; identical to SEQ ID NO: 736
  Synthetic primer

SEQ ID NO: 1073 - DNA, 40 bp, Artificial; identical to SEQ ID NO: 737
  Synthetic primer

SEQ ID NO: 1074 - DNA, 40 bp, Artificial; identical to SEQ ID NO: 832
  Synthetic primer

SEQ ID NO: 1075 - DNA, 40 bp, Artificial; identical to SEQ ID NO: 833
  Synthetic primer

SEQ ID NO: 1076 - DNA, 40 bp, Artificial; identical to SEQ ID NO: 720
  Synthetic primer

SEQ ID NO: 1077 - DNA, 40 bp, Artificial; identical to SEQ ID NO: 721
  Synthetic primer

SEQ ID NO: 1078 - DNA, 40 bp, Artificial; identical to SEQ ID NO: 832
  Synthetic primer

SEQ ID NO: 1079 - DNA, 40 bp, Artificial; identical to SEQ ID NO: 833
  Synthetic primer

SEQ ID NO: 1080 - DNA, 40 bp, Artificial
  Synthetic primer
ggtcgtacgcctcgttcttgacagccatggcccgtcgggc

SEQ ID NO: 1081 - DNA, 40 bp, Artificial
  Synthetic primer
acgaggggggcacgatgccagcccgacgggccatggctgt

SEQ ID NO: 1082 - DNA, 40 bp, Artificial; identical to SEQ ID NO: 832
  Synthetic primer

SEQ ID NO: 1083 - DNA, 40 bp, Artificial; identical to SEQ ID NO: 833
  Synthetic primer

SEQ ID NO: 1084 - DNA, 40 bp, Artificial; identical to SEQ ID NO: 736
  Synthetic primer

SEQ ID NO: 1085 - DNA, 40 bp, Artificial; identical to SEQ ID NO: 737
  Synthetic primer

SEQ ID NO: 1086 - DNA, 40 bp, Artificial; identical to SEQ ID NO: 1080
  Synthetic primer

SEQ ID NO: 1087 - DNA, 40 bp, Artificial; identical to SEQ ID NO: 1081
  Synthetic primer

SEQ ID NO: 1088 - DNA, 40 bp, Artificial; identical to SEQ ID NO: 832
  Synthetic primer

SEQ ID NO: 1089 - DNA, 40 bp, Artificial; identical to SEQ ID NO: 833
  Synthetic primer

SEQ ID NO: 1090 - DNA, 40 bp, Artificial; identical to SEQ ID NO: 736
  Synthetic primer

SEQ ID NO: 1091 - DNA, 40 bp, Artificial; identical to SEQ ID NO: 737
  Synthetic primer

SEQ ID NO: 1092 - DNA, 40 bp, Artificial; identical to SEQ ID NO: 718
  Synthetic primer

SEQ ID NO: 1093 - DNA, 40 bp, Artificial; identical to SEQ ID NO: 719
  Synthetic primer

SEQ ID NO: 1094 - DNA, 40 bp, Artificial; identical to SEQ ID NO: 1080
  Synthetic primer

SEQ ID NO: 1095 - DNA, 40 bp, Artificial; identical to SEQ ID NO: 1081
  Synthetic primer

SEQ ID NO: 1096 - DNA, 40 bp, Artificial; identical to SEQ ID NO: 832
  Synthetic primer

SEQ ID NO: 1097 - DNA, 40 bp, Artificial; identical to SEQ ID NO: 833
  Synthetic primer